(19) United States
(12) Patent Application Publication
(10) Pub. No.: US 2016/0181542 A1
(43) Pub. Date: Jun. 23, 2016

(54) ANTHRACENE DERIVATIVE AND ORGANIC ELECTROLUMINESCENT ELEMENT USING SAME

(71) Applicant: IDEMITSU KOSAN CO., LTD., Tokyo (JP)

(72) Inventors: Masahiro KAWAMURA, Sodegaura-shi, Chiba (JP); Yumiko MIZUKI, Sodegaura-shi, Chiba (JP); Hirokatsu ITO, Sodegaura-shi, Chiba (JP); Tomoharu HAYAMA, Sodegaura-shi, Chiba (JP); Tasuku HAKETA, Sodegaura-shi, Chiba (JP)

(73) Assignee: IDEMITSU KOSAN CO., LTD., Tokyo (JP)

(21) Appl. No.: 14/908,937

(22) PCT Filed: Sep. 3, 2014
(86) PCT No.: PCT/JP2014/004525

§ 371 Date: Jan. 29, 2016

(30) Foreign Application Priority Data
Sep. 6, 2013 (JP) 2013-185630

(51) Int. Cl. H01L 51/00; C07D 307/91; C07C 13/66; C09K 11/02; C09K 11/06

(52) U.S. Cl. CPC H01L 51/0054; C09K 11/025; C09K 11/06; C07C 13/66; H01L 51/0058; C07D 307/91; H01L 51/0073; H01L 51/0055; H01L 51/006; H01L 51/0061; H01L 51/5012

(57) ABSTRACT

An anthracene derivative is represented by the following formula (1). In the formula (1), one of R₁₁ to R₂₀ is used to bond to L₁, and is a single bond. The remainder of R₁₁ to R₂₀ that are not used to bond to L₁ are independently a hydrogen atom, a halogen atom, a cyano group, a substituted or unsubstituted alkyl group including 1 to 20 carbon atoms, or the like. L₁ is a single bond, a substituted or unsubstituted divalent aromatic hydrocarbon group including 6 to 50 ring carbon atoms, or the like. Z has a structure represented by the following formula (2). In the formula (2), one of R₁, R₃, and R₄ is used to bond to L₁, and is a single bond. The remainder of R₁, R₃, and R₄ that are not used to bond to L₁, R₂, and R₅ to R₁₀ are independently a hydrogen atom, a halogen atom, a cyano group, a substituted or unsubstituted alkyl group including 1 to 20 carbon atoms, or the like. At least one pair of groups among R₅ to R₈ that are adjacent to each other are bonded to each other to form a saturated or unsaturated hydrocarbon ring.

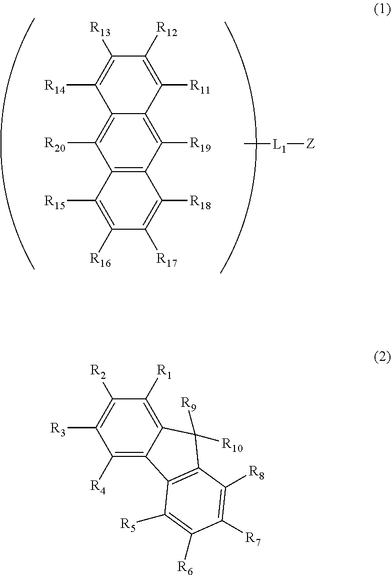

### TECHNICAL FIELD

The invention relates to an anthracene derivative, an organic electroluminescence device that includes the anthracene derivative, and an electronic device that includes the organic electroluminescence device.

### BACKGROUND ART

An organic electroluminescence (EL) device is considered to be a promising inexpensive large full-color display that utilizes solid-state emission, and has been extensively developed. The organic EL device normally includes an emitting layer, and a pair of opposing electrodes that are disposed on either side of the emitting layer. When an electric field is applied between the electrodes, electrons are injected from the cathode, and holes are injected from the anode. The electrons and the holes recombine in the emitting layer to produce an excited state, and the energy is emitted as light when the excited state returns to the ground state.

A known organic EL device has problems in that a high driving voltage is required, and only low luminance and low luminous (emission) efficiency can be achieved as compared with an inorganic light-emitting diode. Moreover, a significant deterioration in characteristics may occur. Therefore, it has been difficult to put the organic EL device to practical use. Although the organic EL device has been improved in recent years, a further improvement in luminous efficiency and the like has been desired. The performance of the organic EL device has been gradually improved through an improvement in organic EL emitting material. It is important to improve the luminous efficiency of the organic EL device in order to reduce the power consumption of a display. Various attempts have been made to improve the luminous efficiency of the organic EL device. However, a further improvement has been desired.

Patent Literature 1 to 3 that aim to address the above problem disclose an organic EL device in which an anthracene derivative that is substituted with benzofluorene is used as an emitting material.

Patent Literature 4 to 6 disclose an anthracene derivative that is substituted with a 3-fluorenyl group or a 4-fluorenyl group and may be used as an emitting material. A decrease in voltage may be achieved using these materials, but a decrease in efficiency occurs. Therefore, a further improvement in efficiency has been desired.

### CITATION LIST

### Patent Literature

### Patent Literature 1: WO2004/061048

### Patent Literature 2: KR-A-2009-0117326

### Patent Literature 3: WO2010/114253

### Patent Literature 4: KR-A-2011-0081698

### Patent Literature 5: JP-A-2009-249378

### Patent Literature 6: JP-A-2007-314506

### SUMMARY OF INVENTION

An object of the invention is to provide a compound that makes it possible to provide an organic electroluminescence device that can be driven at a low voltage and exhibits high luminous efficiency.

One aspect of the invention provides the following compound.

An anthracene derivative represented by the following formula (1),

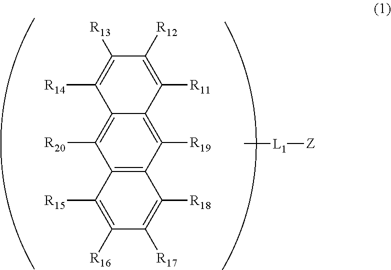

wherein in the formula (1), one of R₁₁ to R₂₀ is used to bond. to L₁, and is a single bond, the remainder of R₁₁ to R₂₀ that are not used to bond to L₁ are independently a hydrogen atom, a halogen atom, a cyano group, a substituted or unsubstituted alkyl group including 1 to 20 carbon atoms, a substituted or unsubstituted alkenyl group including 2 to 20 carbon atoms, a substituted or unsubstituted alkynyl group including 2 to 20 carbon atoms, a substituted or unsubstituted alkoxy group including 1 to 20 carbon atoms, a substituted or unsubstituted alkylthio group including 1 to 20 carbon atoms, a substituted or unsubstituted aryloxy group including 6 to 50 carbon atoms that form a ring (hereinafter referred to as “ring carbon atoms”), a substituted or unsubstituted arylthio group including 6 to 50 ring carbon atoms, an alkylsilyl group that is substituted with a substituted or unsubstituted alkyl group including 1 to 20 carbon atoms, an arylsilyl group that is substituted with a substituted or unsubstituted aryl group including 6 to 50 ring carbon atoms, a substituted or unsubstituted aryl group including 6 to 50 ring carbon atoms, a substituted or unsubstituted heterocyclic group including 5 to 50 atoms that form a ring (hereinafter referred to as “ring atoms”), or a substituted or unsubstituted amino group,
provided that adjacent groups among R₁₁ to R₂₀ are optionally bonded to each other to form a ring, L₁ is a single bond, a substituted or unsubstituted divalent aromatic hydrocarbon group including 6 to 50 ring carbon atoms, or a substituted or unsubstituted divalent heterocyclic group including 5 to 50 ring atoms, and
Z has a structure represented by the following formula (2),

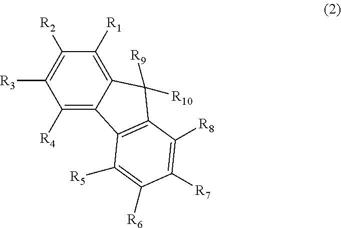

wherein in the formula (2), one of R₁, R₃, and R₄ is used to bond to L₁, and is a single bond, and the remainder of R₁, R₃, and R₄ that are not used to bond to L₁, R₂, and R₅ to R₁₀ are independently a hydrogen atom, a halogen atom, a cyano group, a substituted or unsubstituted alkyl group including 1 to 20 carbon atoms, a substituted or unsubstituted alkenyl group including 2 to 20 carbon atoms, a substituted or unsubstituted alkynyl group including 2 to 20 carbon atoms, a substituted or unsubstituted alkoxy group including 1 to 20 carbon atoms, a substituted or unsubstituted alkylthio group including 1 to 20 carbon atoms, a substituted or unsubstituted aryloxy group including 6 to 50 ring carbon atoms, a substituted or unsubstituted arylthio group including 6 to 50 ring carbon atoms, an alkylsilyl group that is substituted with a substituted or unsubstituted alkyl group including 1 to 20 carbon atoms, an arylsilyl group that is substituted with a substituted or unsubstituted aryl group including 6 to 50 ring carbon atoms, a substituted or unsubstituted aryl group including 6 to 50 ring carbon atoms, a substituted or unsubstituted heterocyclic group including 5 to 50 ring atoms, or a substituted or unsubstituted amino group,
provided that at least one pair of groups among R₅ to R₈ that are adjacent to each other are bonded to each other to form a saturated or unsaturated hydrocarbon ring, and
one of R₁, R₃, and R₄ is bonded directly to one of R₁₁ to R₂₀ when L₁ is a single bond.

The invention thus provides a compound that makes it possible to provide an organic electroluminescence device that can be driven at a low voltage and exhibits high luminous efficiency.

### DESCRIPTION OF EMBODIMENTS

The compound (anthracene derivative) according to one aspect of the invention is represented by the following formula (1)

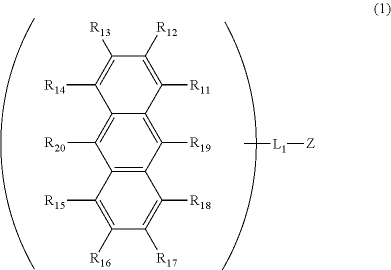

In the formula (1), one of R₁₁ to R₂₀ is used to bond to L₁, and is a single bond.

The remainder of R₁₁ to R₂₀ that are not used to bond to L₁ are independently a hydrogen atom, a halogen atom, a cyano group, a substituted or unsubstituted alkyl group including 1 to 20 carbon atoms, a substituted or unsubstituted alkenyl group including 2 to 20 carbon atoms, a substituted or unsubstituted alkynyl group including 2 to 20 carbon atoms, a substituted or unsubstituted alkoxy group including 1 to 20 carbon atoms, a substituted or unsubstituted alkylthio group including 1 to 20 carbon atoms, a substituted or unsubstituted aryloxy group including 6 to 50 ring carbon atoms, a substituted or unsubstituted arylthio group including 6 to 50 ring carbon atoms, an alkylsilyl group that is substituted with a substituted or unsubstituted alkyl group including 1 to 20 carbon atoms, an arylsilyl group that is substituted with a substituted or unsubstituted aryl group including 6 to 50 ring carbon atoms, a substituted or unsubstituted aryl group including 6 to 50 ring carbon atoms, a substituted or unsubstituted heterocyclic group including 5 to 50 ring atoms, or a substituted or unsubstituted amino group.

Adjacent groups among R₁₁ to R₂₀ are optionally bonded to each other to form a ring.

L₁ is a single bond, a substituted or unsubstituted divalent aromatic hydrocarbon group including 6 to 50 ring carbon atoms, or a substituted or unsubstituted divalent heterocyclic group including 5 to 50 ring atoms.

Z has the structure represented by the following formula (2).

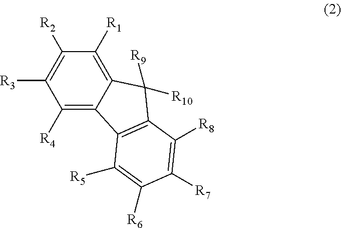

In the formula (2), one of R₁, R₃, and R₄ is used to bond to L₁, and is a single bond.

One of R₁, R₃, and R₄ is bonded directly to one of R₁₁ to R₂₀ when L₁ is a single bond.

The remainder of R₁, R₃, and R₄ that are not used to bond to L₁, R₂, and R₅ to R₁₀ are independently a hydrogen atom, a halogen atom, a cyano group, a substituted or unsubstituted alkyl group including 1 to 20 carbon atoms, a substituted or unsubstituted alkenyl group including 2 to 20 carbon atoms, a substituted or unsubstituted alkynyl group including 2 to 20 carbon atoms, a substituted or unsubstituted alkoxy group including 1 to 20 carbon atoms, a substituted or unsubstituted alkylthio group including 1 to 20 carbon atoms, a substituted or unsubstituted aryloxy group including 6 to 50 ring carbon atoms, a substituted or unsubstituted arylthio group including 6 to 50 ring carbon atoms, an alkylsilyl group that is substituted with a substituted or unsubstituted alkyl group including 1 to 20 carbon atoms, an arylsilyl group that is substituted with a substituted or unsubstituted aryl group including 6 to 50 ring carbon atoms, a substituted or unsubstituted aryl group including 6 to 50 ring carbon atoms, a substituted or unsubstituted heterocyclic group including 5 to 50 ring atoms, or a substituted or unsubstituted amino group.

At least one pair of groups among R₅ to R₈ that are adjacent to each other are bonded to each other to form a saturated or unsaturated hydrocarbon ring. For example, R₅ and R₆, R₆ and R₇, or R₇ and R₈ are bonded to each other to form a hydrocarbon ring. R₅ and R₆ may be bonded to each other to form a hydrocarbon ring, and R₇ and R₈ may be bonded to each other to form a hydrocarbon ring.

The anthracene derivative represented by the formula (1) that has the above structure makes it possible to provide an organic EL device that can be driven at a low voltage and exhibits high luminous efficiency when used to produce an organic EL device.

It is preferable that R₁₂, R₁₉, or R₂₀ among R₁₁ to R₂₀ in the formula (1) be bonded to L₁.

It is preferable that at least one pair of groups among R₅ to R₈ that are adjacent to each other be bonded to each other to form a ring structure represented by the following formula (3).

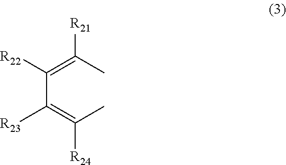

In the formula (3), R₂₁ to R₂₄ are independently the same as defined above in connection with R₂ and R₅ to R₁₀ in the formula (2).

Adjacent groups among R₂₁ to R₂₄ are optionally bonded to each other to form a ring.

Z has preferably a structure among structures respectively represented by the following formulas (4) to (7).

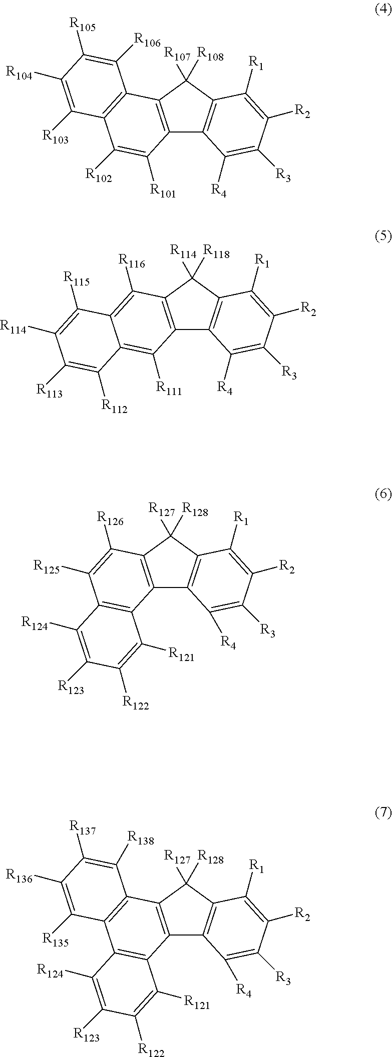

In the formulas (4) to (7), one of R₁, R₃, and R₄ is used to bond to L₁, and is a single bond.

The remainder of R₁, R₃, and R₄ that are not used to bond to L₁, R₂, R₁₀₁ to R₁₀₈, R₁₁₁ to R₁₁₈, R₁₂₁ to R₁₂₈, and R₁₃₁ to R₁₄₀ are independently the same as defined above in connection with R₂ and R₅ to R₁₀ in the formula (2).

It is preferable that at least one of R₁₁ to R₂₀ in the formula (1) that is not used to bond to L₁ be a substituted or unsubstituted aryl group including 6 to 50 ring carbon atoms, or a substituted or unsubstituted heterocyclic group including 5 to 50 ring atoms. It is more preferable that R₂₀ be a substituted or unsubstituted aryl group including 6 to 50 ring carbon atoms, or a substituted or unsubstituted heterocyclic group including 5 to 50 ring atoms.

The anthracene derivative represented by the formula (1) is preferably represented by any of the following formulas (8) to (11).

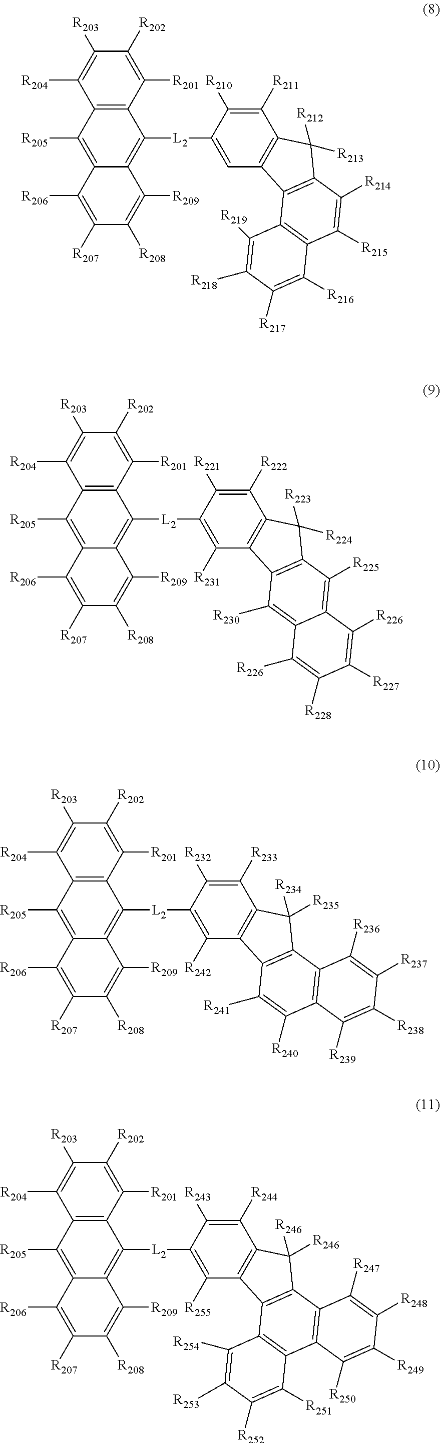

In the formulas (8) to (11), R₂₀₁ to R₂₀₉ are independently the same as defined above in connection with R₁₁ to R₂₀ in the formula (1) that are not used to bond to L₁.

R₂₁₀ to R₂₂₀, R₂₂₁ to R₂₃₁, R₂₃₂ to R₂₄₂, and R₂₄₃ to R₂₅₅ are independently the same as defined above in connection with R₂ and R₅ to R₁₀ in the formula (2).

L₂ is the same as defined above in connection with L₁ in the formula (1).

It is preferable that R₂₀₅ in the formulas (8) to (11) be a substituted or unsubstituted aryl group including 6 to 50 ring carbon atoms, or a substituted or unsubstituted heterocyclic group including 5 to 50 ring atoms.

The anthracene derivative represented by the formula (1) is preferably represented by any of the following formulas (12) to (15).

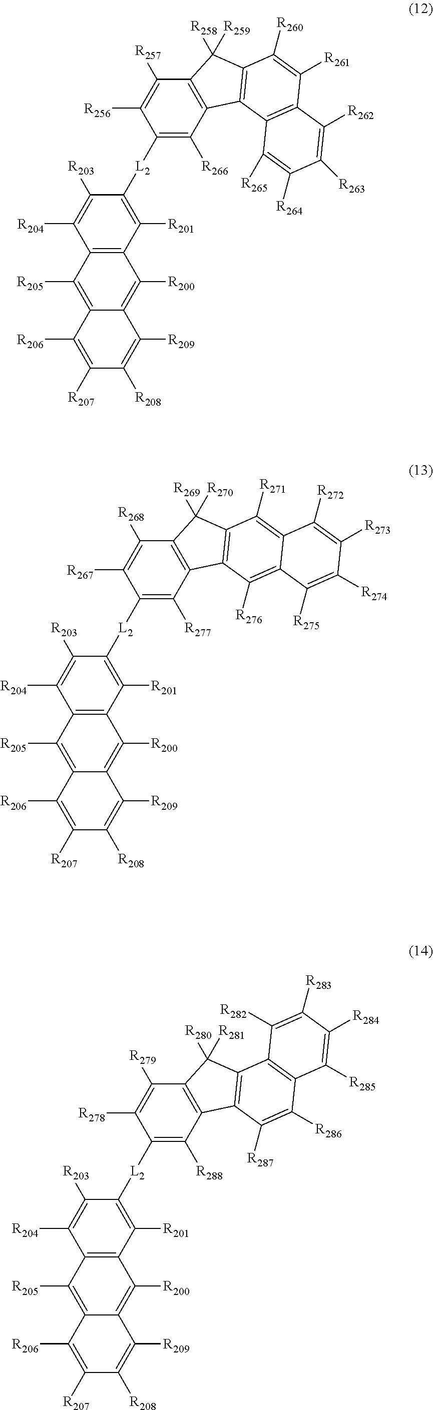

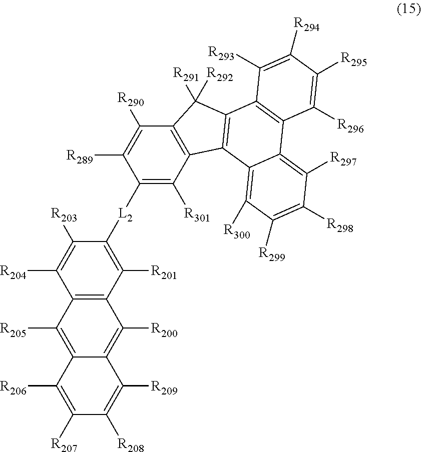

In the formulas (12) to (15), R₂₀₀, R₂₀₁, and R₂₀₃ to R₂₀₉ are independently the same as defined above in connection with R₁₁ to R₂₀ in the formula (1) that are not used to bond to L₁.

R₂₅₆ to R₂₆₅, R₂₆₇ to R₂₇₇, R₂₇₈ to R₂₈₈, and R₂₈₉ to R₃₀₁ are independently the same as defined above in connection with R₂ and R₅ to R₁₀ in the formula (2).

L₂ is the same as defined above in connection with L₁ in the formula (1).

It is preferable that one or more selected from R₂₀₀ and R₂₀₅ in the formulas (12) to (15) be independently a substituted or unsubstituted aryl group including 6 to 50 ring carbon atoms, or a substituted or unsubstituted heterocyclic group including 5 to 50 ring atoms.

The term “ring carbon atom” used herein refers to a carbon atom that forms a saturated ring, an unsaturated ring, or an aromatic ring. The term “ring atom” used herein refers to a carbon atom and a heteroatom (e.g., N, O, S, and Si) that form a heteroring (including a saturated ring, an unsaturated ring, and an aromatic ring).

The expression “a to b carbon atoms” used in connection with the expression “substituted or unsubstituted XX group including a to b carbon atoms” refers to the number of carbon atoms when the XX group is unsubstituted, and excludes the number of carbon atoms included in a substituent when the XX group is substituted.

Examples of a substituent when the expression “substituted or unsubstituted” is used include a halogen atom, a cyano group, an alkyl group, an alkenyl group, an alkynyl group, an alkoxy group, an alkylthio group, an aryloxy group, an arylthio group, an alkylsilyl group, an arylsilyl group, an aryl group, a heterocyclic group, an amino group, and the like (described later) unless otherwise specified. The above substituents may be further substituted with a substituent among the above substituents.

The term “unsubstituted” used in connection with the expression “substituted or unsubstituted” means that the group is not substituted with a substituent (i.e., a hydrogen atom is bonded).

The term “hydrogen atom” used herein includes isotopes of hydrogen that differ in the number of neutrons (i.e., protium, deuterium, and tritium).

R₁₁ to R₂₀, L₁, R₁ to R₁₀, R₂₁ to R₂₄, R₁₀₁ to R₁₀₈, R₁₁₁ to R₁₁₈, R₁₂₁ to R₁₂₈, R₁₃₁ to R₁₄₀, R₂₀₁ to R₂₀₉, R₂₁₀ to R₂₂₀, R₂₂₁ to R₂₃₁, R₂₃₂ to R₂₄₂, R₂₄₃ to R₂₅₅, R₂₀₀, R₂₀₁, R₂₀₃ to R₂₀₉, R₂₅₆ to R₂₆₆, R₂₆₇ to R₂₇₇, R₂₇₈ to R₂₈₈, and R₂₈₉ to R₃₀₁ included in the above compounds, and each substituent when the expression “substituted or unsubstituted” is used are described in detail below.

Examples of the halogen atom include a fluorine atom, a chlorine atom, a bromine atom, an iodine atom, and the like. Among these, a fluorine atom is preferable.

Examples of the alkyl group including 1 to 20 (preferably 1 to 10, more preferably 1 to 8, still more preferably 1 to 6, and particularly preferably 1 to 4) carbon atoms include a methyl group, an ethyl group, a propyl group, an isopropyl group, an n-butyl group, an s-butyl group, an isobutyl group, a t-butyl group, an n-pentyl group, an n-hexyl group, an n-heptyl group, an n-octyl group, and the like.

A methyl group, an ethyl group, a propyl group, an isopropyl group, an n-butyl group, an s-butyl group, an isobutyl group, a t-butyl group, an n-pentyl group, and an n-hexyl group are preferable as the alkyl group.

Examples of the substituted alkyl group include an alkyl group that is substituted with an aryl group (described later) (i.e., a substituent formed by combining an alkylene group and an aryl group (e.g., phenylmethyl group and 2-phenylisopropyl group)).

Examples of the alkenyl group including 2 to 20 (preferably 2 to 10) carbon atoms include a vinyl group, an allyl group, a 1-butenyl group, a 2-butenyl group, a 3-butenyl group, a 1,3-butanedienyl group, a 1-methylvinyl group, a 1-methylallyl group, a 1,1-dimethylallyl group, a 2-methylallyl group, a 1,2-dimethylallyl group, and the like.

Examples of the substituted alkenyl group include a styryl group, a 2,2-diphenylvinyl group, a 1,2-diphenylvinyl group, a 1-phenylallyl group, a 2-phenylallyl group, a 3-phenylallyl group, a 3,3-diphenylallyl group, a 1-phenyl-1-butenyl group, a 3-phenyl-1-butenyl group, and the like.

Examples of the alkynyl group including 2 to 20 (preferably 2 to 10) carbon atoms include a propargyl group, a 3-pentynyl group, and the like.

The alkoxy group including 1 to 20 (preferably 1 to 10, more preferably 1 to 8, and particularly preferably 1 to 4) carbon atoms is a group represented by —OY. Examples of Y include the groups mentioned above as examples of the alkyl group. Examples of the alkoxy group include a methoxy group and an ethoxy group.

The alkylthio group including 1 to 20 (preferably 1 to 10, more preferably 1 to 8, and particularly preferably 1 to 4) carbon atoms is a group represented by —SY. Examples of Y include the groups mentioned above as examples of the alkyl group.

The aryloxy group including 6 to 50 (preferably 6 to 20, and more preferably 6 to 12) ring carbon atoms is a group represented by —OAr. Examples of Ar include the groups mentioned below as examples of the aryl group. Examples of the aryloxy group include a phenoxy group.

The arylthio group including 6 to 50 (preferably 6 to 20, and more preferably 6 to 12) ring carbon atoms is a group represented by —SAr. Examples of Ar include the groups mentioned below as examples of the aryl group.

Examples of the alkylsilyl group that is substituted with a substituted or unsubstituted alkyl group including 1 to 20 carbon atoms include a silyl group that is substituted with one, two, or three alkyl groups. Examples of the alkyl group include those mentioned above.

Specific examples of the alkylsilyl group include a trimethylsilyl group, a triethylsilyl group, a tri-n-butylsilyl group, a tri-n-octylsilyl group, a triisobutylsilyl group, a dimethylethylsilyl group, a dimethylisopropylsilyl group, a dimethyl-n-propylsilyl group, a dimethyl-n-butylsilyl group, a dimethyl-t-butylsilyl group, a diethylisopropylsilyl group, a vinyldimethylsilyl group, a propyldimethylsilyl group, a triisopropylsilyl group, and the like. The silyl group may be substituted with three alkyl groups that are either identical or different.

Examples of the arylsilyl group that is substituted with a substituted or unsubstituted aryl group including 6 to 50 ring carbon atoms include a silyl group that is substituted with one, two, or three aryl groups. Examples of the aryl group include those mentioned below. The arylsilyl group may have a structure in which an aryl group and an alkyl group are bonded to the silicon atom.

Examples of the arylsilyl group include an arylsilyl group, an alkylarylsilyl group, a dialkylarylsilyl group, a diarylsilyl group, an alkyldiarylsilyl group, and a triarylsilyl group. A plurality of aryl groups or a plurality of alkyl groups may be either identical or different.

Examples of the dialkylarylsilyl group include a dialkylarylsilyl group that includes two alkyl groups among those mentioned above, and one aryl group among those mentioned below. The number of carbon atoms included in the dialkylarylsilyl group is preferably 8 to 30. The two alkyl groups may be either identical or different.

Examples of the alkyldiarylsilyl group include an alkyldiarylsilyl group that includes one alkyl group among those mentioned above, and two aryl groups among those mentioned below. The number of carbon atoms included in the alkyldiarylsilyl group is preferably 13 to 30. The two aryl groups may be either identical or different.

Examples of the triarylsilyl group include a triarylsilyl group that includes three aryl groups among those mentioned below. The number of carbon atoms included in the triarylsilyl group is preferably 18 to 30. The three aryl groups may be either identical or different.

Examples of the arylsilyl group include a phenyldimethylsilyl group, a diphenylmethylsilyl group, a diphenyl-t-butylsilyl group, and a triphenylsilyl group.

Examples of the aryl group (aromatic hydrocarbon group) including 6 to 50 (preferably 6 to 30, more preferably 6 to 20, and particularly preferably 6 to 12) ring carbon atoms include a phenyl group, a 1-naphthyl group, a 2-naphthyl group, a 1-anthryl group, a 2-anthryl group, a 9-anthryl group, a 1-phenanthryl group, a 2-phenanthryl group, a 3-phenanthryl group, a 4-phenanthryl group, a 9-phenanthryl group, a 1-naphthacenyl group, a 2-naphthacenyl group, a 9-naphthacenyl group, a 1-pyrenyl group, a 2-pyrenyl group, a 4-pyrenyl group, a 6-chrysenyl group, a 1-benzo[c]phenanthryl group, a 2-benzo[c]phenanthryl group, a 3-benzo[c]phenanthryl group, a 4-benzo[c]phenanthryl group, a 5-benzo[c]phenanthryl group, a 6-benzo[c]phenanthryl group, a 1-benzo[g]chrysenyl group, a 2-benzo[g]chrysenyl group, a 3-benzo[g]chrysenyl group, a 4-benzo[g]chrysenyl group, a 5-benzo[g]chrysenyl group, a 6-benzo[g]chrysenyl group, a 7-benzo[g]chrysenyl group, an 8-benzo[g]chrysenyl group, a 9-benzo[g]chrysenyl group, a 10-benzo[g]chrysenyl group, a 11-benzo[g]chrysenyl group, a 12-benzo[g]chrysenyl group, a 13-benzo[g]chrysenyl group, a 14-benzo[g]chrysenyl group, a 1-benzo[a]anthryl group, a 2-benzo[a]anthryl group, a 3-benzo[a]anthryl group, a 4-benzo[a]anthryl group, a 5-benzo[a]anthryl group, a 6-benzo[a]anthryl group, a 7-benzo[a]anthryl group, a 8-benzo[a]anthryl group, a 9-benzo[a]anthryl group, a 10-benzo[a]anthryl group, a 11-benzo[a]anthryl group, a 12-benzo[a]anthryl group, a 13-benzo[a]anthryl group, a 14-benzo[a]anthryl group, a 1-triphenylenyl group, a 2-triphenylenyl group, a 1-fluorenyl group, a 2-fluorenyl group, a 3-fluorenyl group, a 4-fluorenyl group, a 9-fluorenyl group, a benzofluorenyl group, a dibenzofluorenyl group, a 2-biphenylyl group, a 3-biphenylyl group, a 4-biphenylyl group, a p-terphenyl-4-yl group, a p-terphenyl-3-yl group, a p-terphenyl-2-yl group, an m-terphenyl-4-yl group, an m-terphenyl-3-yl group, an m-terphenyl-2-yl group, and the like.

Among these, a phenyl group, a 1-naphthyl group, a 2-naphthyl group, a 1-phenanthryl group, a 2-phenanthryl group, a 3-phenanthryl group, a 4-phenanthryl group, a 9-phenanthryl group, a 1-fluorenyl group, a 2-fluorenyl group, a 3-fluorenyl group, a 4-fluorenyl group, a 5-benzo[c]phenanthryl group, a 4-benzo[a]anthryl group, a 7-benzo[a]anthryl group, a 10-benzo[g]chrysenyl group, a 1-triphenylenyl group, and a 2-triphenylenyl group are preferable.

It is preferable that a 1-fluorenyl group, a 2-fluorenyl group, a 3-fluorenyl group, a 4-fluorenyl group, and a 9-fluorenyl group have a structure in which a substituted or unsubstituted alkyl group including 1 to 20 carbon atoms (see above), a substituted or unsubstituted aryl group including 6 to 18 carbon atoms (see above), or a heterocyclic group including 5 to 20 atoms (see below) is bonded to the carbon atom at position 9.

It is preferable that these aryl groups be further substituted with an aryl group including 6 to 30 ring carbon atoms, a heterocyclic group including 5 to 20 ring atoms, an alkyl group including 1 to 20 carbon atoms, a silyl group that is substituted with an alkyl group including 1 to 20 carbon atoms, a cyano group, or a halogen atom.

The term “aryl group (aromatic hydrocarbon group)” used herein refers to a hydrocarbon group that exhibits aromaticity and includes a single ring (non-fused aryl group) or a plurality of rings (fused aryl group).

The term “fused aryl group” refers to an aryl group in which two or more ring structures are fused. The term “non-fused aryl group” refers to an aryl group other than the fused aryl group.

Examples of the fused aryl group include a fused aryl group including 10 to 50 (preferably 10 to 30, and more preferably 10 to 20) ring carbon atoms, such as a 1-naphthyl group, a 2-naphthyl group, a 1-anthryl group, a 2-anthryl group, a 9-anthryl group, a 1-phenanthryl group, a 2-phenanthryl group, a 3-phenanthryl group, a 4-phenanthryl group, a 9-phenanthryl group, a 1-naphthacenyl group, a 2-naphthacenyl group, a 9-naphthacenyl group, a 1-pyrenyl group, a 2-pyrenyl group, a 4-pyrenyl group, a 6-chrysenyl group, a 5-benzo[c]phenanthryl group, a 4-benzo[a]anthryl group, a 7-benzo[a]anthryl group, a 10-benzo[g]chrysenyl group, a 1-triphenylenyl group, and a 2-triphenylenyl group.

Among these, a 1-naphthyl group, a 2-naphthyl group, a 1-phenanthryl group, a 2-phenanthryl group, a 3-phenanthryl group, a 4-phenanthryl group, a 9-phenanthryl group, a 5-benzo[c]phenanthryl group, a 4-benzo[a]anthryl group, a 7-benzo[a]anthryl group, a 10-benzo[g]chrysenyl group, a 1-triphenylenyl group, and a 2-triphenylenyl group are preferable.

Examples of the divalent aromatic hydrocarbon group include a group obtained by removing one or more hydrogen atoms from the aryl group.

Examples of the heterocyclic group including 5 to 50 (preferably 5 to 30, more preferably 5 to 20, and particularly preferably 5 to 12) ring atoms include a 1-pyrrolyl group, a 2-pyrrolyl group, a 3-pyrrolyl group, a pyrazinyl group, a 2-pyridinyl group, a 3-pyridinyl group, a 4-pyridinyl group, a 1-indolyl group, a 2-indolyl group, a 3-indolyl group, a 4-indolyl group, a 5-indolyl group, a 6-indolyl group, a 7-indolyl group, a 1-isoindolyl group, a 2-isoindolyl group, a 3-isoindolyl group, a 4-isoindolyl group, a 5-isoindolyl group, a 6-isoindolyl group, a 7-isoindolyl group, a 2-furyl group, a 3-furyl group, a 2-benzofuranyl group, a 3-benzofuranyl group, a 4-benzofuranyl group, a 5-benzofuranyl group, a 6-benzofuranyl group, a 7-benzofuranyl group, a 1-isobenzofuranyl group, a 3-isobenzofuranyl group, a 4-isobenzofuranyl group, a 5-isobenzofuranyl group, a 6-isobenzofuranyl group, a 7-isobenzofuranyl group, a 1-dibenzofuranyl group, a 2-dibenzofuranyl group, a 3-dibenzofuranyl group, a 4-dibenzofuranyl group, a 1-dibenzo thiophenyl group, a 2-dibenzo thiophenyl group, a 3-dibenzo thiophenyl group, a 4-dibenzo thiophenyl group, a 2-quinolyl group, a 3-quinolyl group, a 4-quinolyl group, a 5-quinolyl group, a 6-quinolyl group, a 7-quinolyl group, a 8-quinolyl group, a 1-isoquinolyl group, a 3-isoquinolyl group, a 4-isoquinolyl group, a 5-isoquinolyl group, a 6-isoquinolyl group, a 7-isoquinolyl group, a 8-isoquinolyl group, a 2-quinoxalinyl group, a 5-quinoxalinyl group, a 6-quinoxalinyl group, a 1-carbazolyl group, a 2-carbazolyl group, a 3-carbazolyl group, a 4-carbazolyl group, a 9-carbazolyl group, a 1-phenanthridinyl group, a 2-phenanthridinyl group, a 3-phenanthridinyl group, a 4-phenanthridinyl group, a 6-phenanthridinyl group, a 7-phenanthridinyl group, a 8-phenanthridinyl group, a 9-phenanthridinyl group, a 10-phenanthridinyl group, a 1-acridinyl group, a 2-acridinyl group, a 3-acridinyl group, a 4-acridinyl group, a 9-acridinyl group, a 1,7-phenanthrolin-2-yl group, a 1,7-phenanthrolin-3-yl group, a 1,7-phenanthrolin-4-yl group, 1,7-phenanthrolin-5-yl group, a 1,7-phenanthrolin-6-yl group, a 1,7-phenanthrolin-8-yl group, a 1,7-phenanthrolin-9-yl group, a 1,7-phenanthrolin-10-yl group, a 1,8-phenanthrolin-2-yl group, a 1,8-phenanthrolin-3-yl group, a 1,8-phenanthrolin-4-yl group, a 1,8-phenanthrolin-5-yl group, a 1,8-phenanthrolin-6-yl group, a 1,8-phenanthrolin-7-yl group, a 1,8-phenanthrolin-9-yl group, a 1,8-phenanthrolin-10-yl group, a 1,9-phenanthrolin-2-yl group, a 1,9-phenanthrolin-3-yl group, a 1,9-phenanthrolin-4-yl group, a 1,9-phenanthrolin-5-yl group, a 1,9-phenanthrolin-6-yl group, a 1,9-phenanthrolin-7-yl group, a 1,9-phenanthrolin-8-yl group, a 1,9-phenanthrolin-10-yl group, a 1,10-phenanthrolin-2-yl group, a 1,10-phenanthrolin-3-yl group, a 1,10-phenanthrolin-4-yl group, a 1,10-phenanthrolin-5-yl group, a 2,9-phenanthrolin-1-yl group, a 2,9-phenanthrolin-3-yl group, a 2,9-phenanthrolin-4-yl group, a 2,9-phenanthrolin-5-yl group, a 2,9-phenanthrolin-6-yl group, a 2,9-phenanthrolin-7-yl group, a 2,9-phenanthrolin-8-yl group, a 2,9-phenanthrolin-10-yl group, a 2,8-phenanthrolin-1-yl group, a 2,8-phenanthrolin-3-yl group, a 2,8-phenanthrolin-4-yl group, a 2,8-phenanthrolin-5-yl group, a 2,8-phenanthrolin-6-yl group, a 2,8-phenanthrolin-7-yl group, a 2,8-phenanthrolin-9-yl group, a 2,8-phenanthrolin-10-yl group, a 2,7-phenanthrolin-1-yl group, a 2,7-phenanthrolin-3-yl group, a 2,7-phenanthrolin-4-yl group, a 2,7-phenanthrolin-5-yl group, a 2,7-phenanthrolin-6-yl group, a 2,7-phenanthrolin-8-yl group, a 2,7-phenanthrolin-9-yl group, a 2,7-phenanthrolin-10-yl group, a 1-phenazinyl group, a 2-phenazinyl group, a 1-phenothiadinyl group, a 2-phenothiadinyl group, a 3-phenothiadinyl group, a 4-phenothiadinyl group, a 10-phenothiadinyl group, a 1-phenoxadinyl group, a 2-phenoxadinyl group, a 3-phenoxadinyl group, a 4-phenoxadinyl group, a 10-phenoxadinyl group, a 2-oxazolyl group, a 4-oxazolyl group, a 5-oxazolyl group, a 2-oxadiazolyl group, a 5-oxadiazolyl group, a 3-furazanyl group, a 2-thienyl group, a 3-thienyl group, a 2-methylpyrrol-1-yl group, a 2-methylpyrrol-3-yl group, a 2-methylpyrrol-4-yl group, a 2-methylpyrrol-5-yl group, a 3-methylpyrrol-1-yl group, a 3-methylpyrrol-2-yl group, a 3-methylpyrrol-4-yl group, a 3-methylpyrrol-5-yl group, a 2-t-butyl pyrrole-4-yl group, a 3-(2-phenylpropyl)pyrrol-1-yl group, a 2-methyl-1-indolyl group, a 4-methyl-1-indolyl group, a 2-methyl-3-indolyl group, a 4-methyl-3-indolyl group, a 2-t-butyl-1-indolyl group, a 4-t-butyl-1-indolyl group, a 2-t-butyl-3-indolyl group, a 4-t-butyl-3-indolyl group, a 1-benzimidazolyl group, a 2-benzimidazolyl group, a 4-benzimidazolyl group, a 5-benzimidazolyl group, a 6-benzimidazolyl group, a 7-benzimidazolyl group, a 2-imidazo[1,2-a]pyridinyl group, a 3-imidazo[1,2-a]pyridinyl group, a 5-imidazo[1,2-a]pyridinyl group, a 6-imidazo[1,2-a]pyridinyl group, a 7-imidazo[1,2-a]pyridinyl group, a 8-imidazo[1,2-a]pyridinyl group, a benzimidazol-2-on-1-yl group, a benzimidazol-2-on-3-yl group, a benzimidazol-2-on-4-yl group, a benzimidazol-2-on-5-yl group, a benzimidazol-2-on-6-yl group, a benzimidazol-2-on-7-yl group, and the like.

Among these, a 1-dibenzofuranyl group, a 2-dibenzofuranyl group, a 3-dibenzofuranyl group, a 4-dibenzofuranyl group, a 1-dibenzothiophenyl group, a 2-dibenzothiophenyl group, a 3-dibenzothiophenyl group, a 4-dibenzothiophenyl group, a 1-carbazolyl group, a 2-carbazolyl group, a 3-carbazolyl group, a 4-carbazolyl group, a 9-carbazolyl group, a 1-benzimidazolyl group, a 2-benzimidazolyl group, a 4-benzimidazolyl group, a 5-benzimidazolyl group, a 6-benzimidazolyl group, a 7-benzimidazolyl group, a 2-imidazo[1,2-a]pyridinyl group, a 3-imidazo[1,2-a]pyridinyl group, a 5-imidazo[1,2-a]pyridinyl group, a 6-imidazo[1,2-a]pyridinyl group, a 7-imidazo[1,2-a]pyridinyl group, a 8-imidazo[1,2-a]pyridinyl group, a 2-pyridinyl group, a 3-pyridinyl group, a 4-pyridinyl group, a 1,10-phenanthrolin-2-yl group, a 1,10-phenanthrolin-3-yl group, a 1,10-phenanthrolin-4-yl group, a 1,10-phenanthrolin-5-yl group, a benzimidazol-2-on-1-yl group, a benzimidazol-2-on-3-yl group, a benzimidazol-2-on-4-yl group, a benzimidazol-2-on-5-yl group, a benzimidazol-2-on-6-yl group, and a benzimidazol-2-on-7-yl group are preferable, and a 1-dibenzofuranyl group, a 2-dibenzofuranyl group, a 3-dibenzofuranyl group, a 4-dibenzofuranyl group, a 1-dibenzothiophenyl group, a 2-dibenzothiophenyl group, a 3-dibenzothiophenyl group, a 4-dibenzothiophenyl group, a 1-carbazolyl group, a 2-carbazolyl group, a 3-carbazolyl group, a 4-carbazolyl group, and a 9-carbazolyl group are particularly preferable.

It is preferable that these heterocyclic groups be further substituted with an aryl group including 6 to 30 ring carbon atoms, a heterocyclic group including 5 to 20 ring atoms, an alkyl group including 1 to 20 carbon atoms, a silyl group that is substituted with an alkyl group including 1 to 20 carbon atoms, a cyano group, or a halogen atom.

The term “heterocyclic group” includes a monocyclic heteroaromatic ring group, a fused heteroaromatic ring group in which a plurality of heteroaromatic rings are fused, and a fused heteroaromatic ring group in which an aromatic hydrocarbon ring and a heteroaromatic ring are fused.

Examples of a fused heterocyclic group including 8 to 30 (preferably 8 to 20) ring atoms include a dibenzofuranyl group, a dibenzothiophenyl group, a carbazolyl group, and the like.

Examples of the divalent heterocyclic group include a group obtained by removing one or more hydrogen atoms from the heterocyclic group.

The amino group is represented by —NHR_{W} or —N(R_{W})₂ (wherein the two R_{w} are either identical or different). Examples of R_{W} include the groups mentioned above as examples of the aryl group including 6 to 50 ring carbon atoms and the heterocyclic group including 5 to 50 ring atoms. A phenylamino group and a diphenylamino group are preferable as the amino group.

Examples of the anthracene derivative according to one aspect of the invention are as follows. Note that the anthracene derivative according to one aspect of the invention is not limited to the following examples.

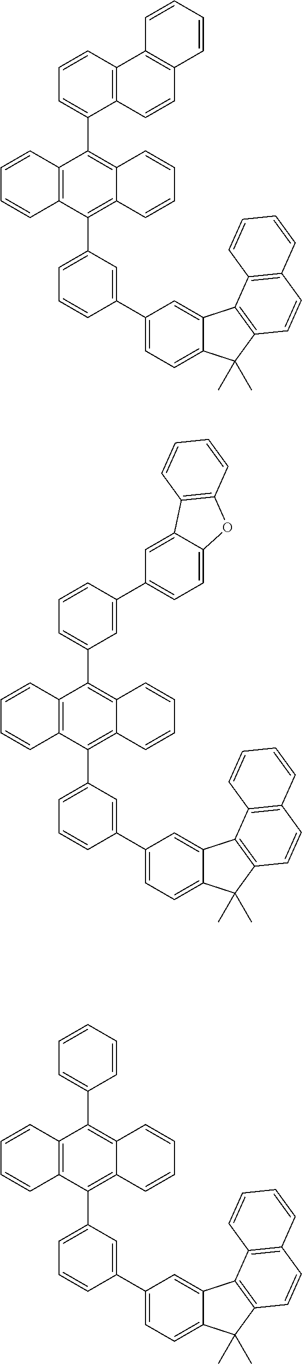

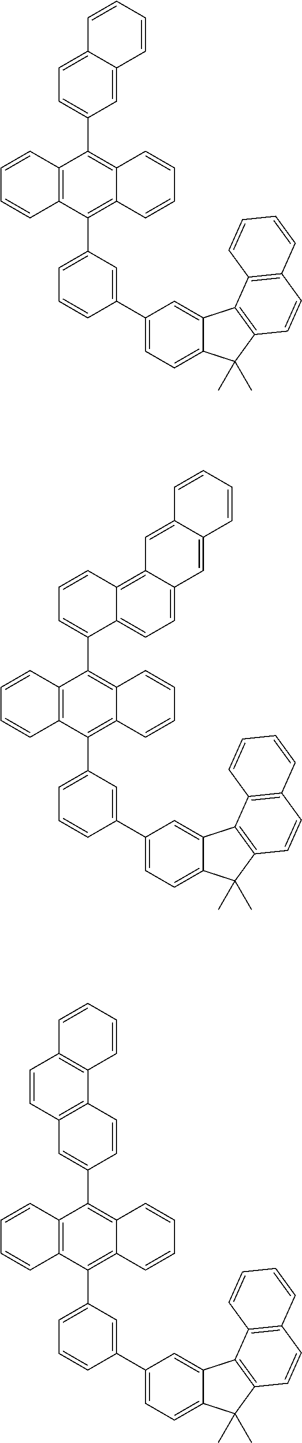

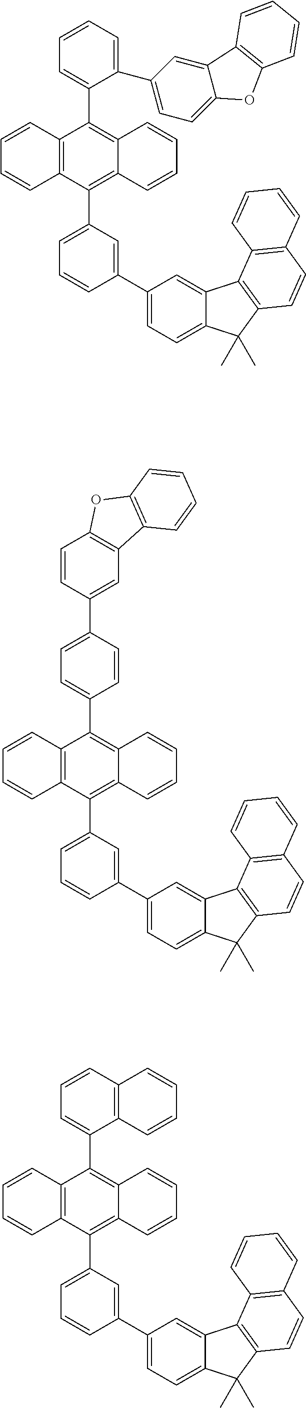

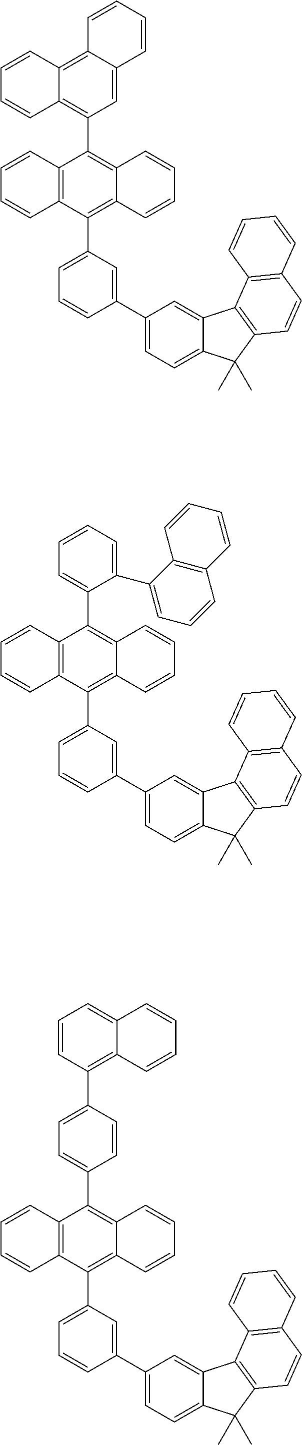

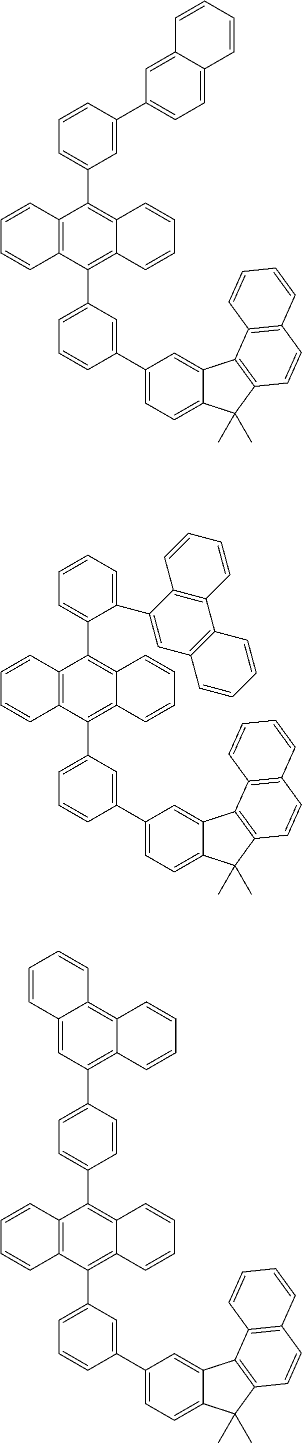

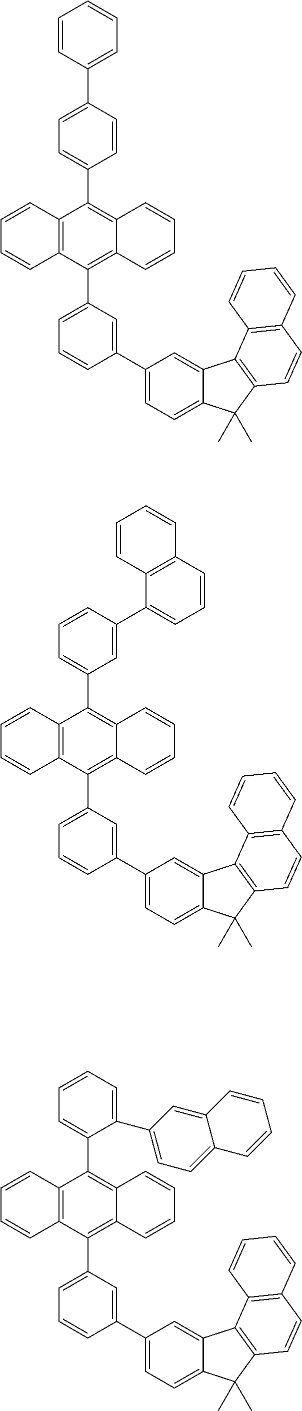

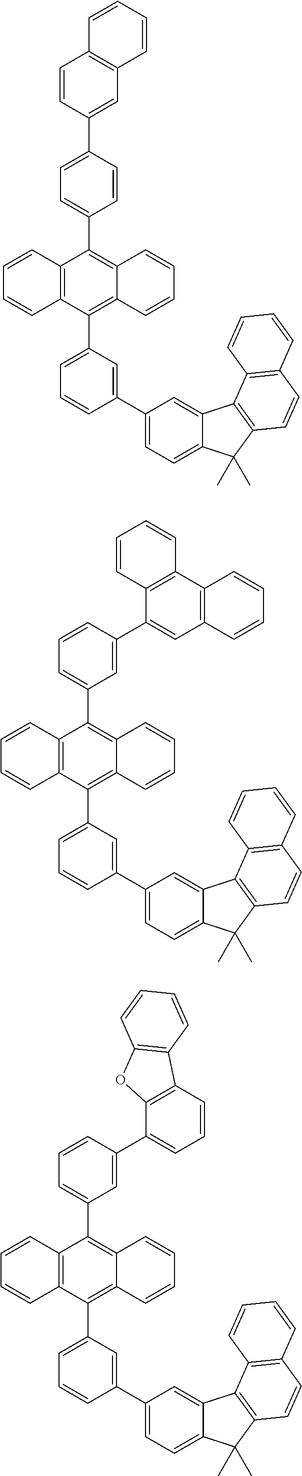

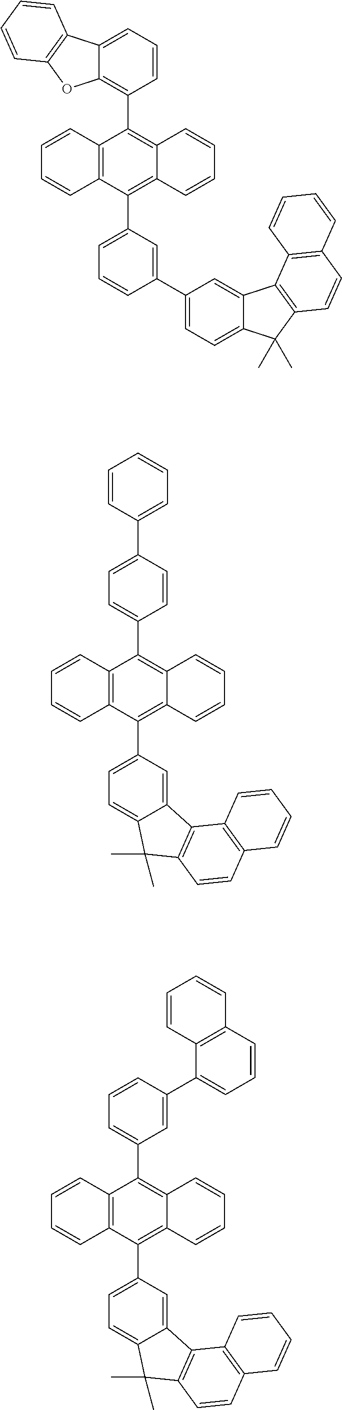

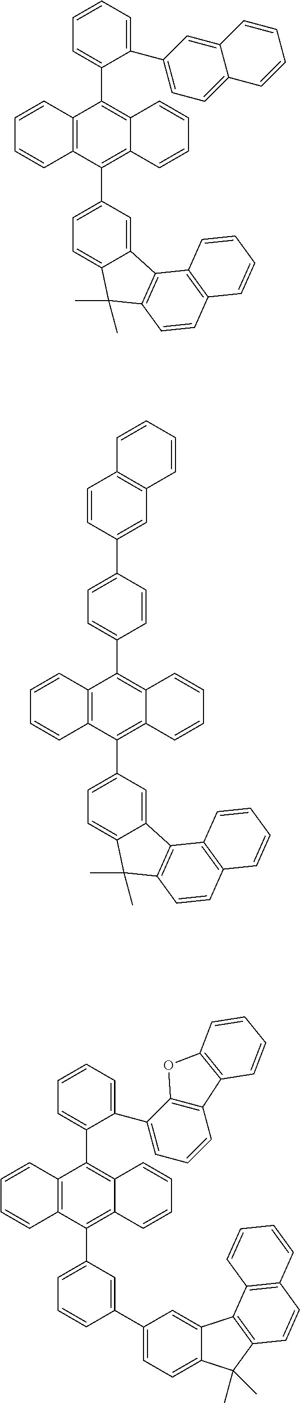

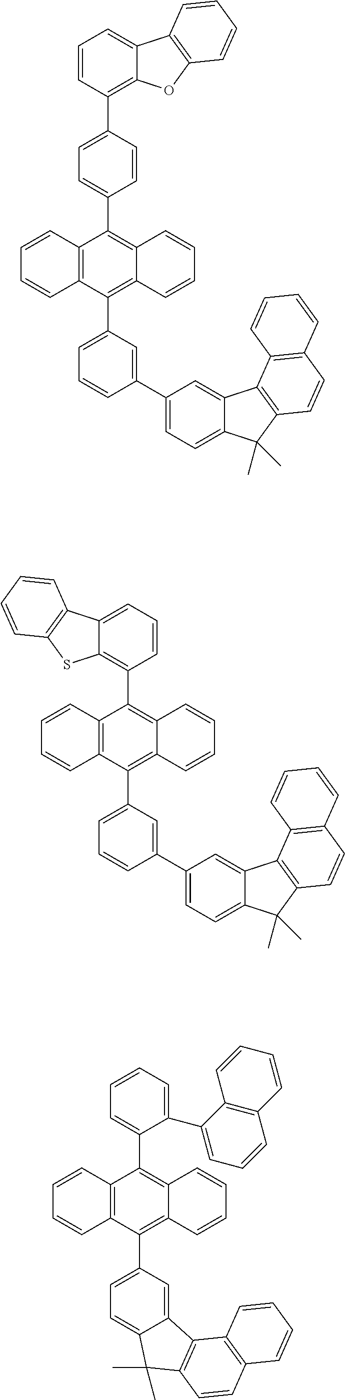

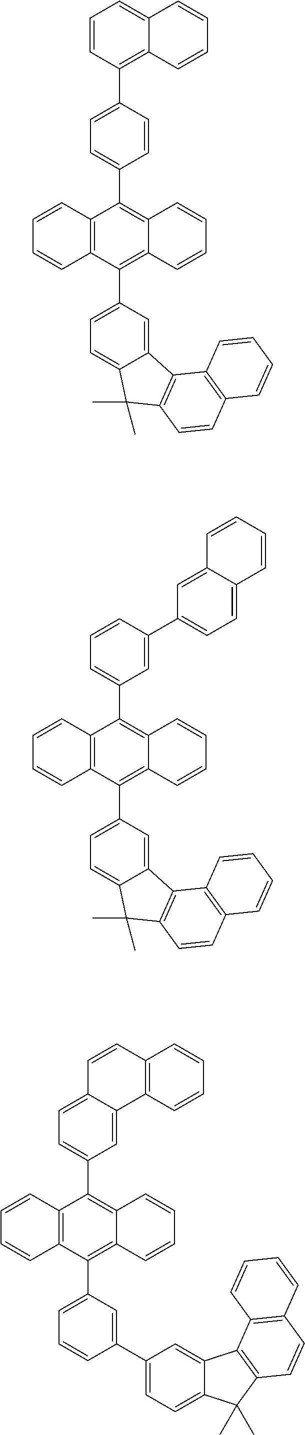

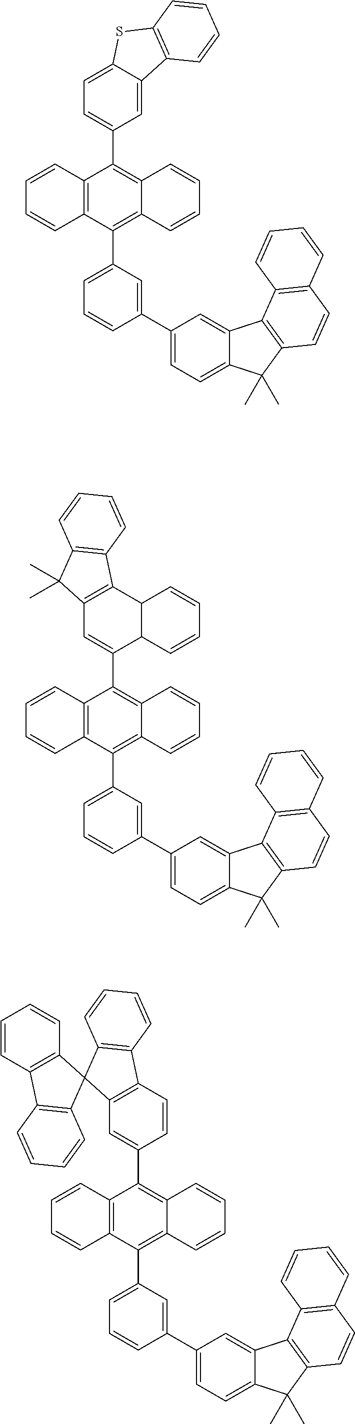

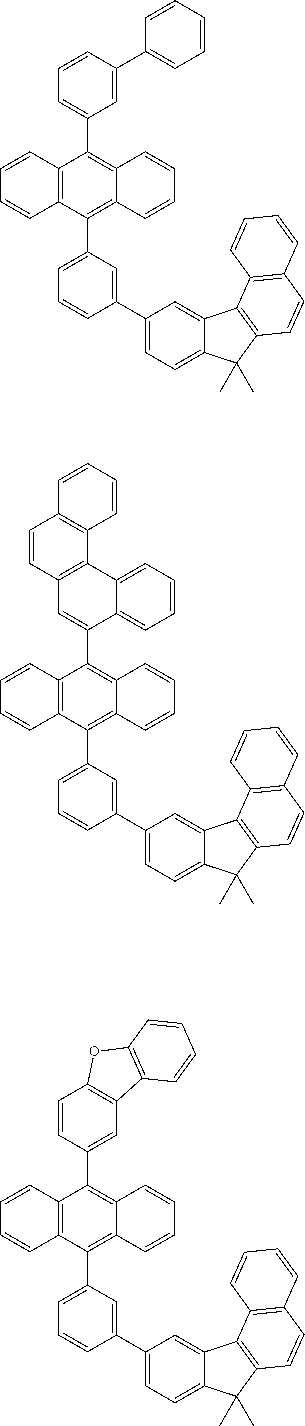

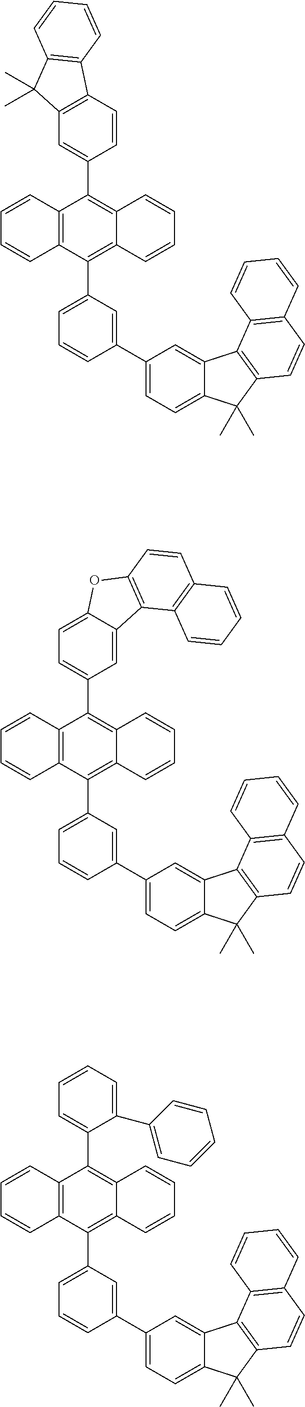

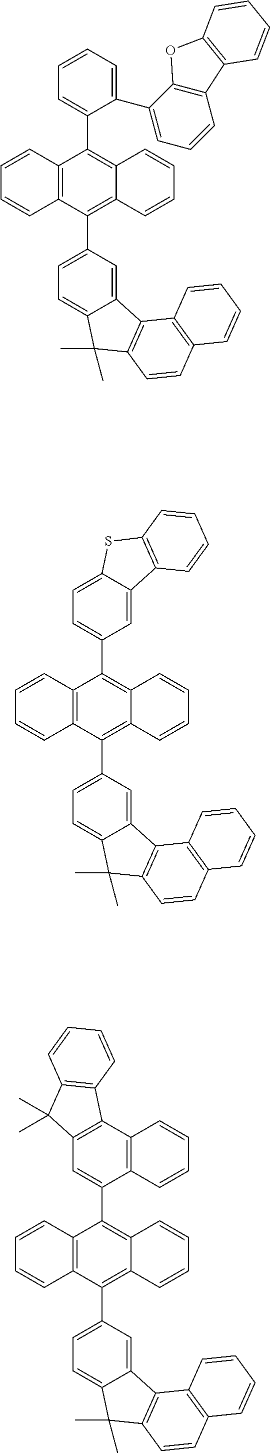

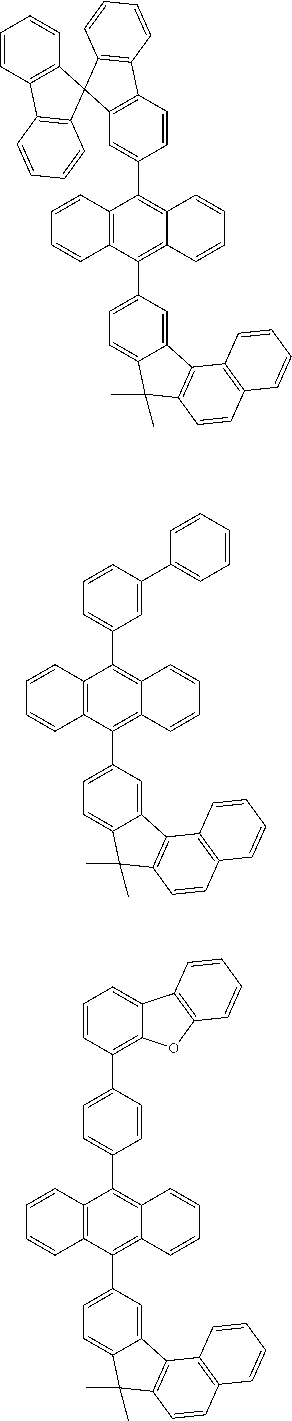

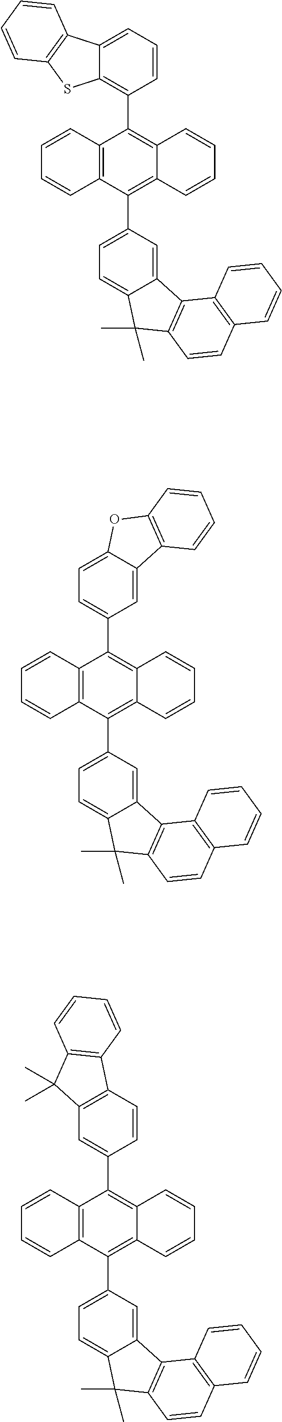

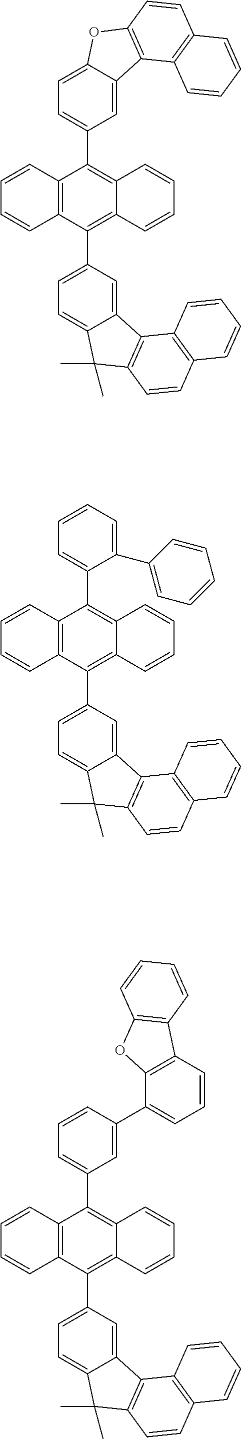

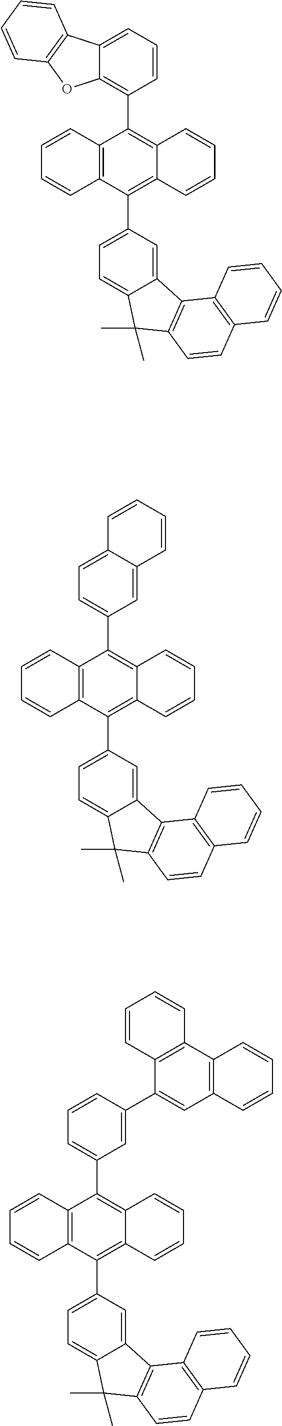

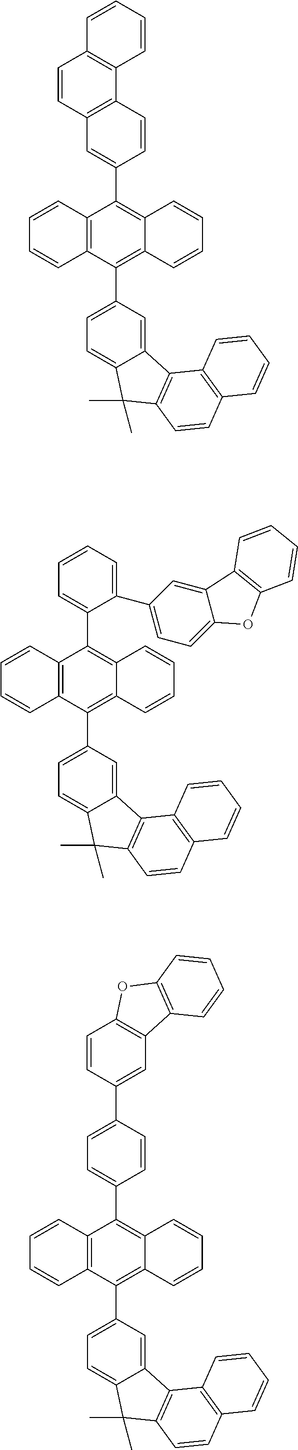

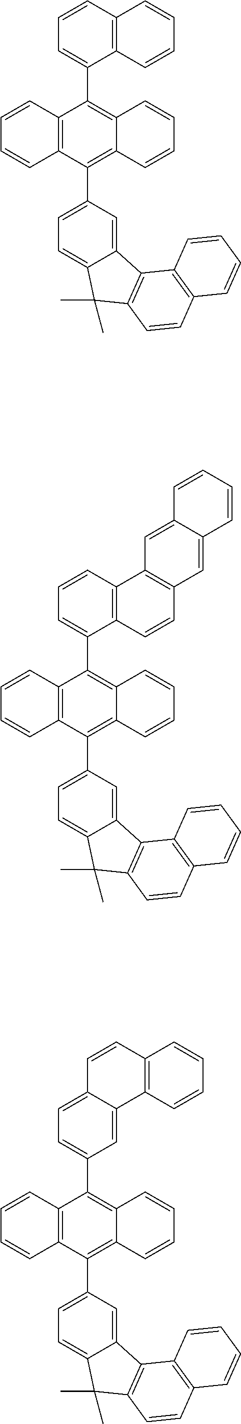

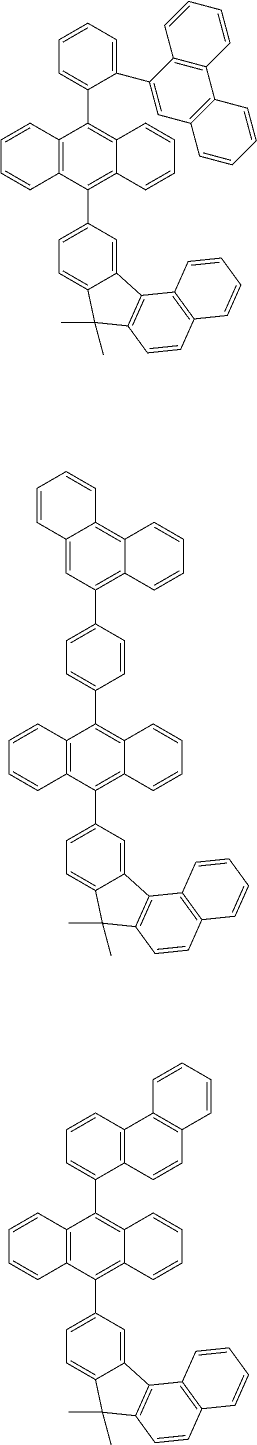

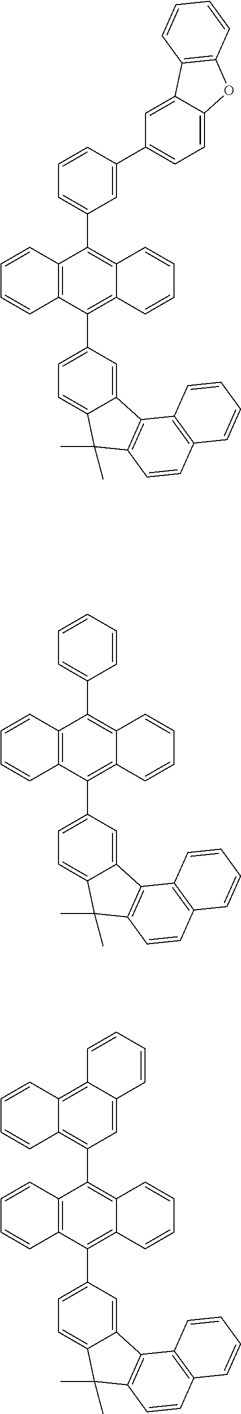

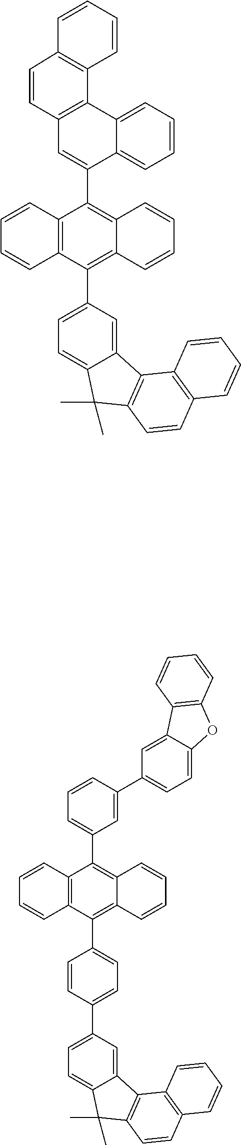

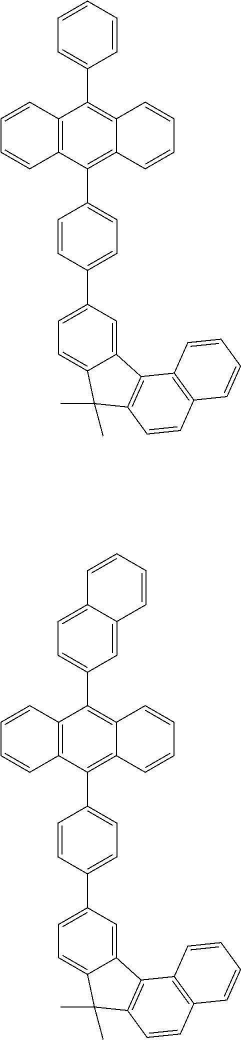

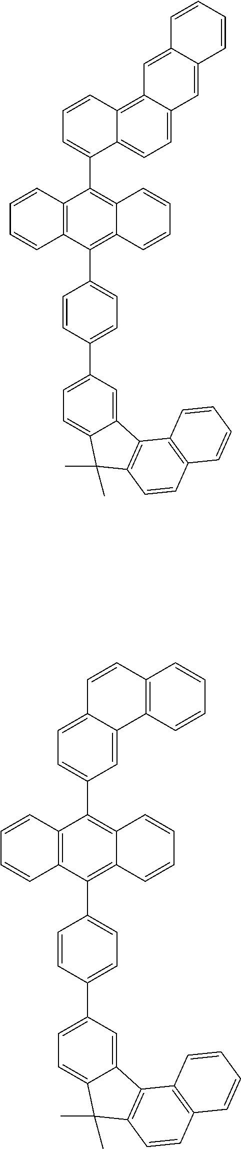

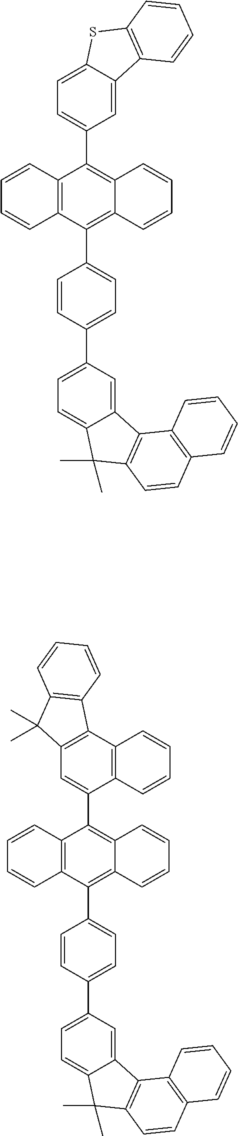

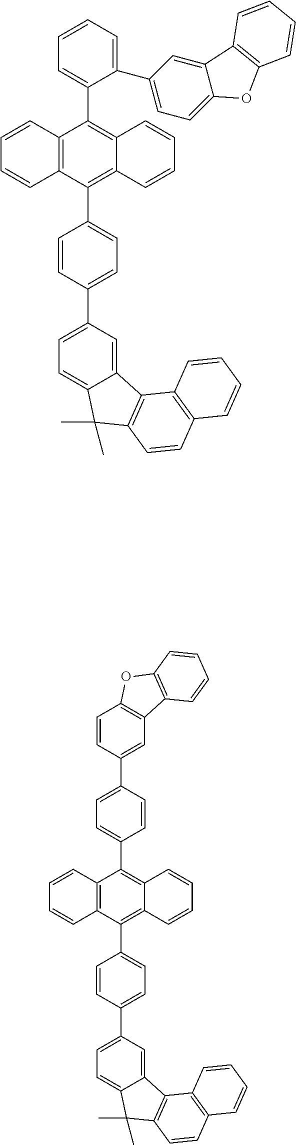 [SMILES: CC1(C)c2ccc(-c3ccc(-c4c5ccccc5c(-c5ccccc5-c5ccc6oc7ccccc7c6c5)c5ccccc45)cc3)cc2-c2c1ccc1ccccc21 ; CC1(C)c2ccc(-c3ccc(-c4c5ccccc5c(-c5ccc(-c6ccc7oc8ccccc8c7c6)cc5)c5ccccc45)cc3)cc2-c2c1ccc1ccccc21]

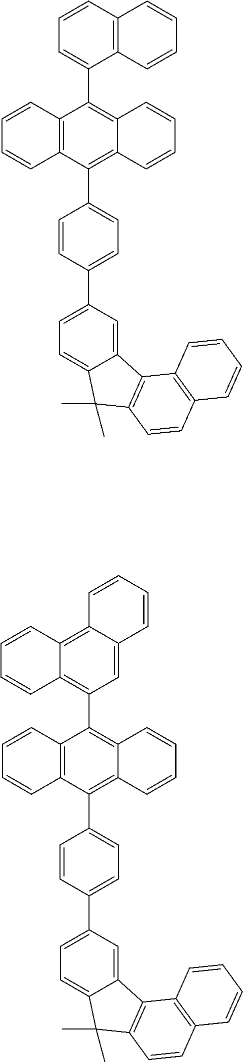

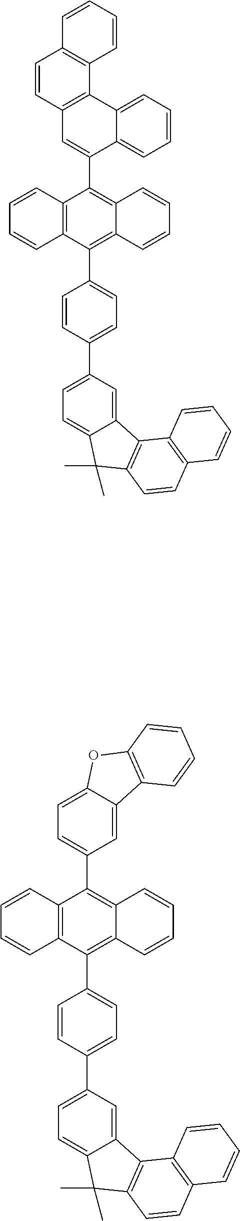

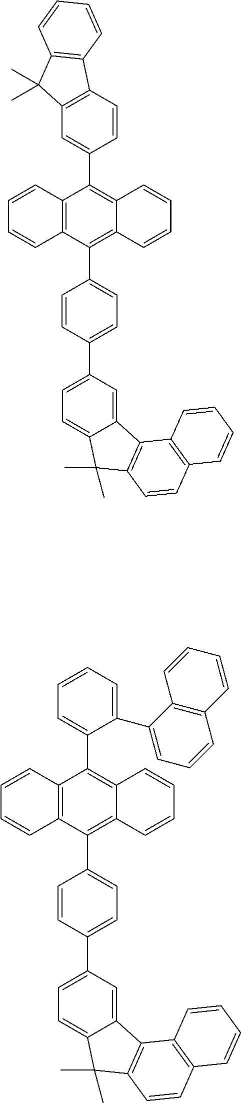

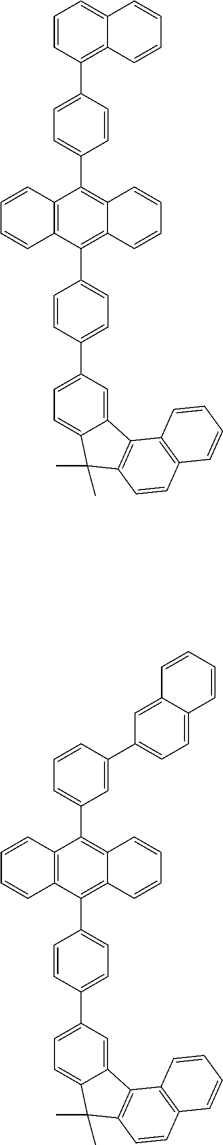

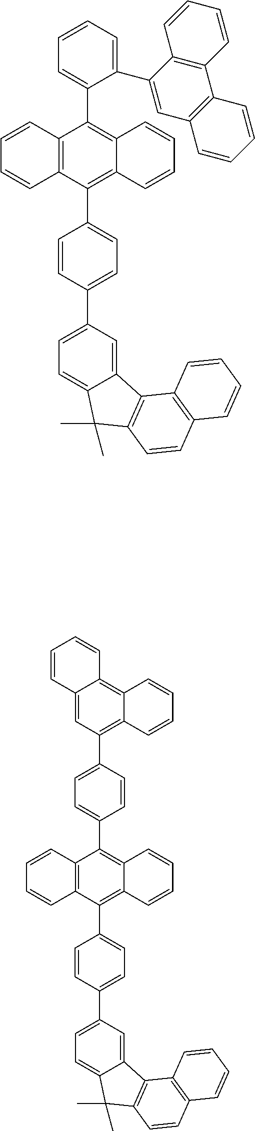

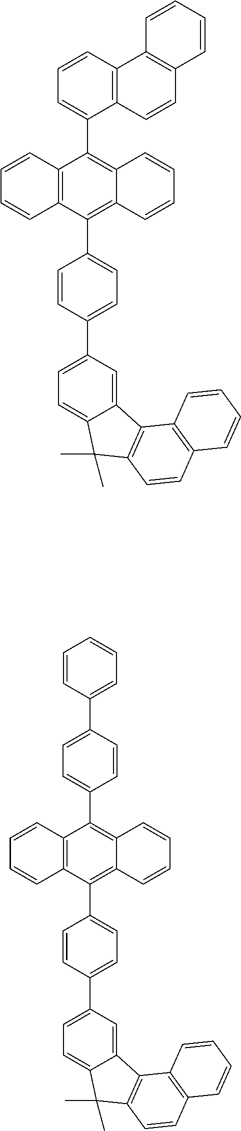

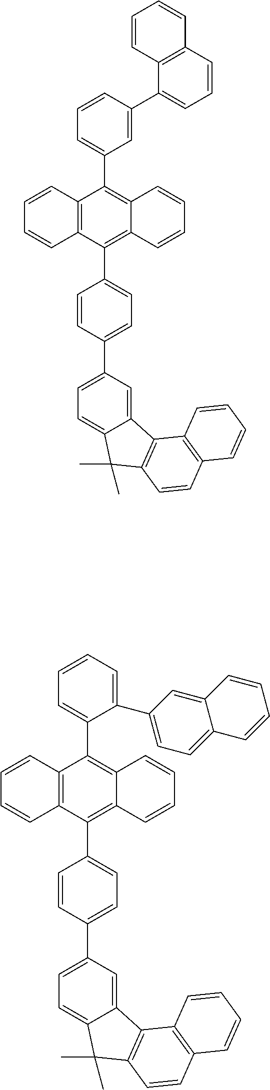

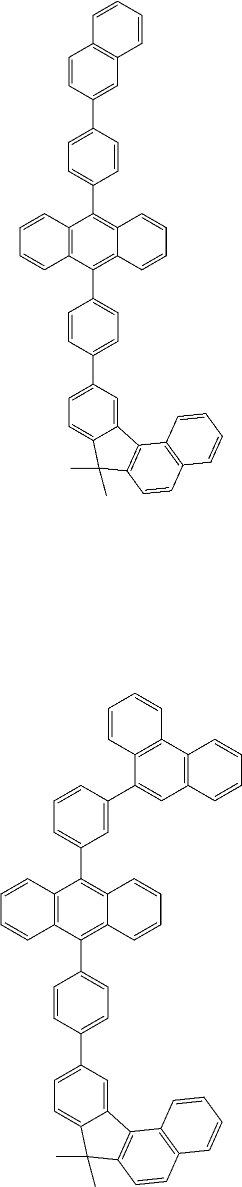

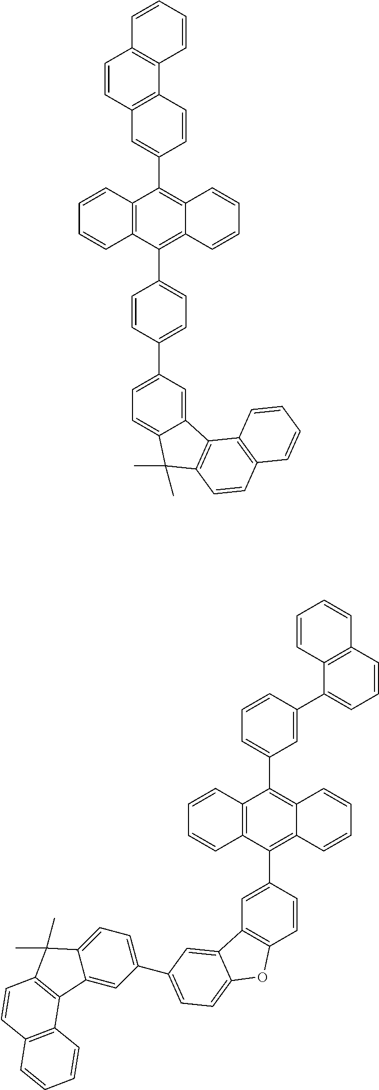

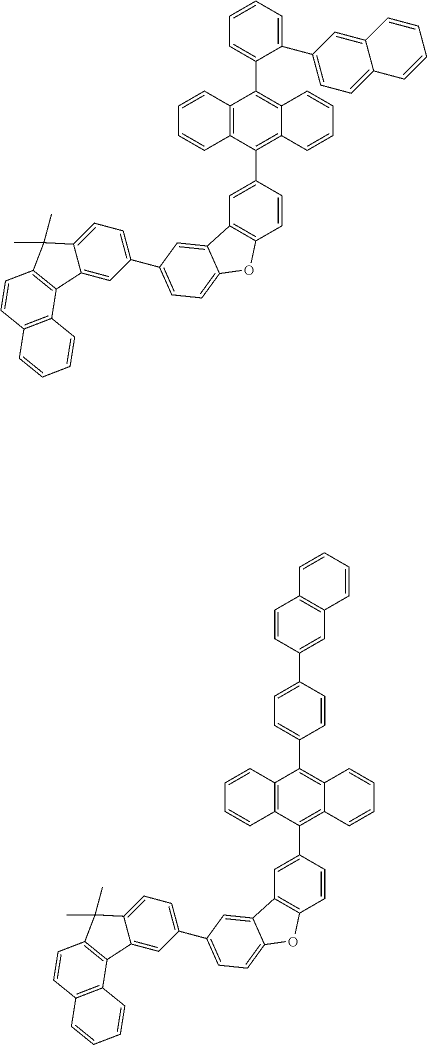 [SMILES: CC1(C)c2ccc(-c3ccc4oc5ccc(-c6c7ccccc7c(-c7ccccc7-c7ccc8ccccc8c7)c7ccccc67)cc5c4c3)cc2-c2c1ccc1ccccc21 ; CC1(C)c2ccc(-c3ccc4oc5ccc(-c6c7ccccc7c(-c7ccc(-c8ccc9ccccc9c8)cc7)c7ccccc67)cc5c4c3)cc2-c2c1ccc1ccccc21]

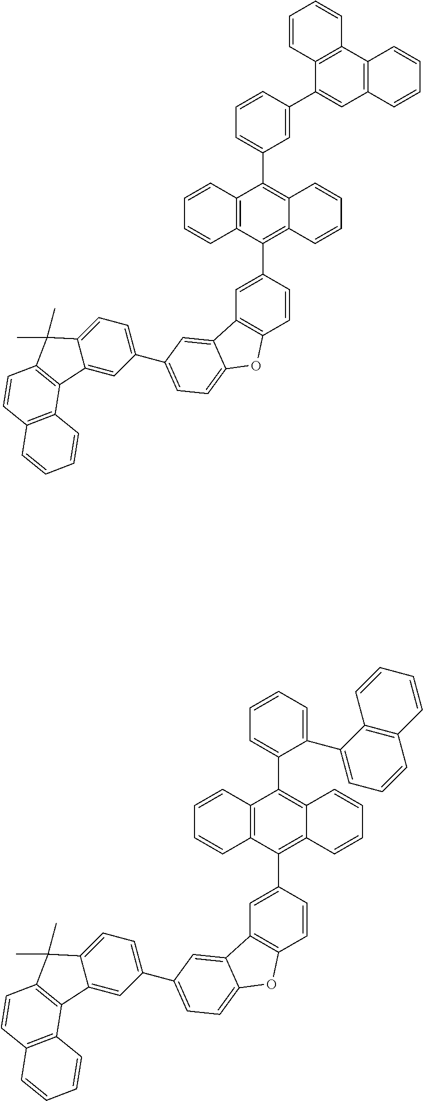 [SMILES: CC1(C)c2ccc(-c3ccc4oc5ccc(-c6c7ccccc7c(-c7cccc(-c8cc9ccccc9c9ccccc89)c7)c7ccccc67)cc5c4c3)cc2-c2c1ccc1ccccc21 ; CC1(C)c2ccc(-c3ccc4oc5ccc(-c6c7ccccc7c(-c7ccccc7-c7cccc8ccccc78)c7ccccc67)cc5c4c3)cc2-c2c1ccc1ccccc21]

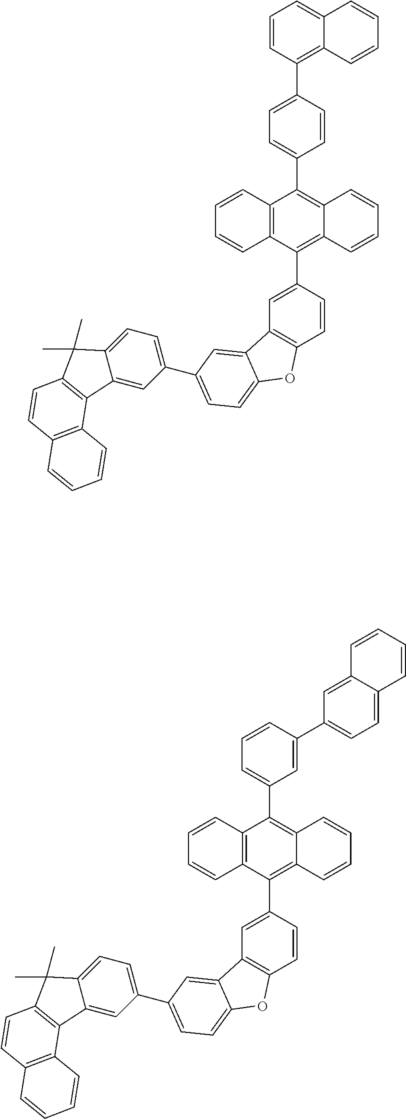 [SMILES: CC1(C)c2ccc(-c3ccc4oc5ccc(-c6c7ccccc7c(-c7ccc(-c8cccc9ccccc89)cc7)c7ccccc67)cc5c4c3)cc2-c2c1ccc1ccccc21 ; CC1(C)c2ccc(-c3ccc4oc5ccc(-c6c7ccccc7c(-c7cccc(-c8ccc9ccccc9c8)c7)c7ccccc67)cc5c4c3)cc2-c2c1ccc1ccccc21]

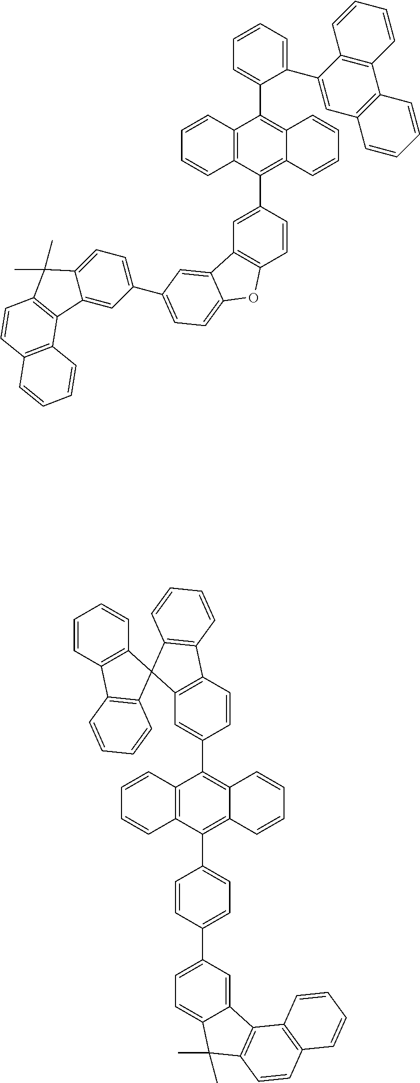

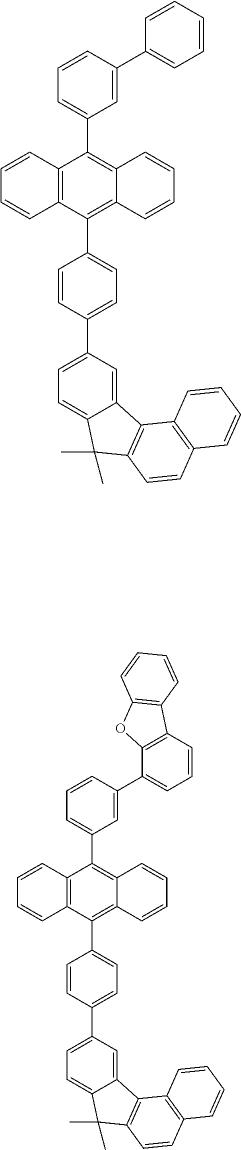

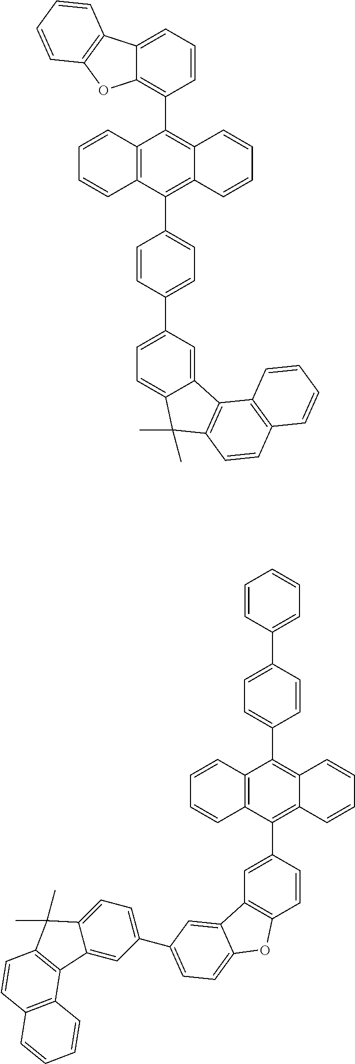 [SMILES: CC1(C)c2ccc(-c3ccc(-c4c5ccccc5c(-c5cccc6c5oc5ccccc56)c5ccccc45)cc3)cc2-c2c1ccc1ccccc21 ; CC1(C)c2ccc(-c3ccc4oc5ccc(-c6c7ccccc7c(-c7ccc(-c8ccccc8)cc7)c7ccccc67)cc5c4c3)cc2-c2c1ccc1ccccc21]

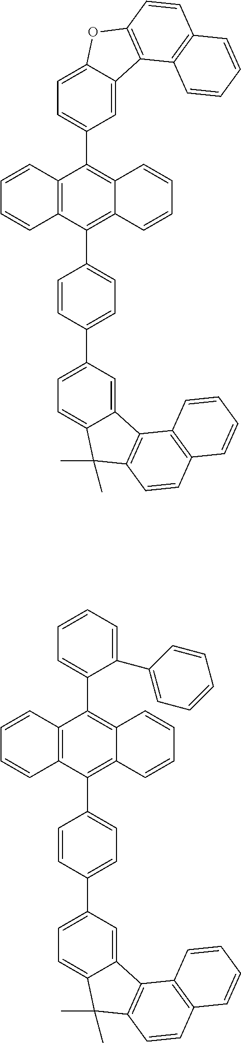

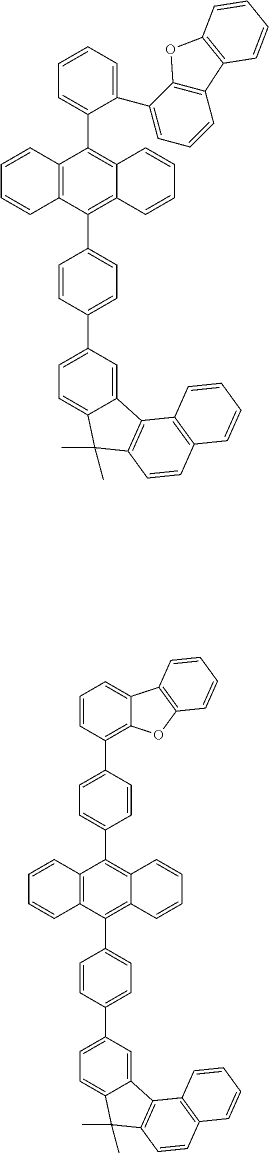 [SMILES: CC1(C)c2ccc(-c3ccc(-c4c5ccccc5c(-c5ccccc5-c5cccc6c5oc5ccccc56)c5ccccc45)cc3)cc2-c2c1ccc1ccccc21 ; CC1(C)c2ccc(-c3ccc(-c4c5ccccc5c(-c5ccc(-c6cccc7c6oc6ccccc67)cc5)c5ccccc45)cc3)cc2-c2c1ccc1ccccc21]

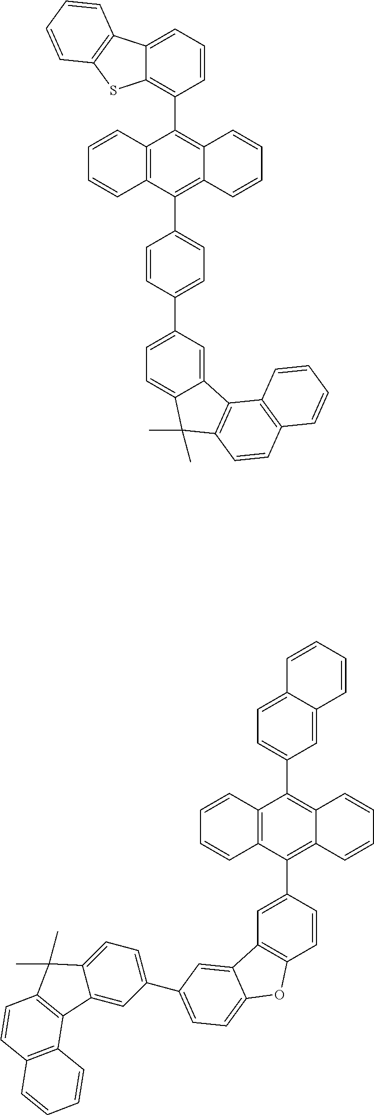 [SMILES: CC1(C)c2ccc(-c3ccc(-c4c5ccccc5c(-c5cccc6c5sc5ccccc56)c5ccccc45)cc3)cc2-c2c1ccc1ccccc21 ; CC1(C)c2ccc(-c3ccc4oc5ccc(-c6c7ccccc7c(-c7ccc8ccccc8c7)c7ccccc67)cc5c4c3)cc2-c2c1ccc1ccccc21]

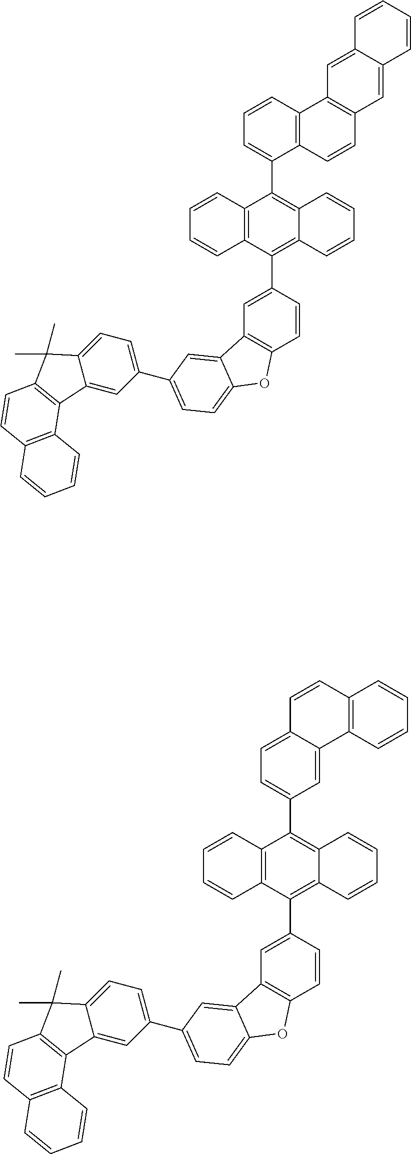 [SMILES: CC1(C)c2ccc(-c3ccc4oc5ccc(-c6c7ccccc7c(-c7cccc8c7ccc7cc9ccccc9cc78)c7ccccc67)cc5c4c3)cc2-c2c1ccc1ccccc21 ; CC1(C)c2ccc(-c3ccc4oc5ccc(-c6c7ccccc7c(-c7ccc8ccc9ccccc9c8c7)c7ccccc67)cc5c4c3)cc2-c2c1ccc1ccccc21]

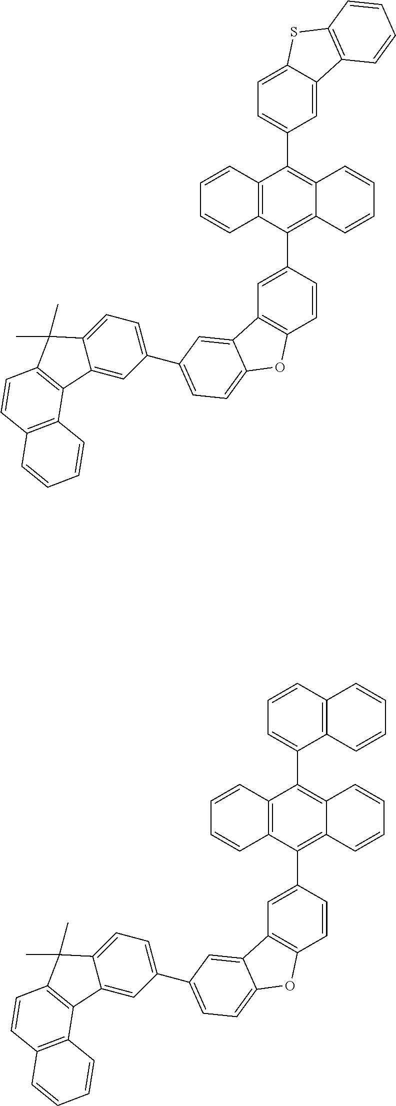 [SMILES: CC1(C)c2ccc(-c3ccc4oc5ccc(-c6c7ccccc7c(-c7ccc8sc9ccccc9c8c7)c7ccccc67)cc5c4c3)cc2-c2c1ccc1ccccc21 ; CC1(C)c2ccc(-c3ccc4oc5ccc(-c6c7ccccc7c(-c7cccc8ccccc78)c7ccccc67)cc5c4c3)cc2-c2c1ccc1ccccc21]

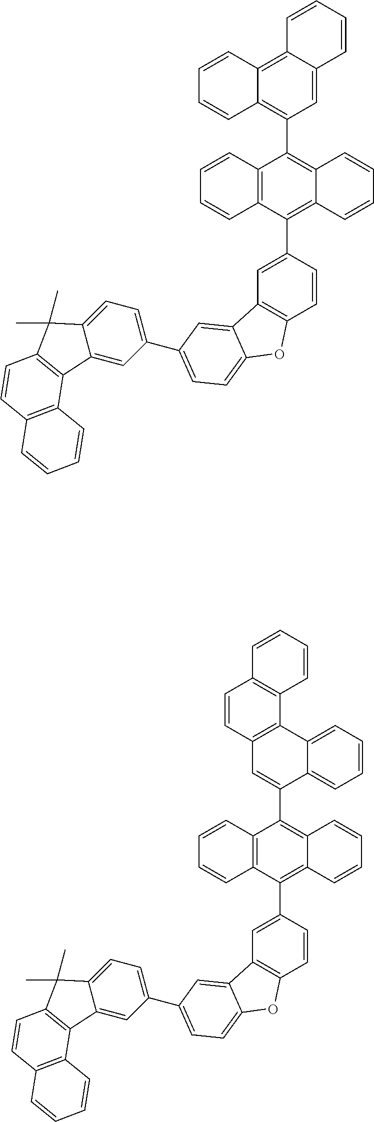 [SMILES: CC1(C)c2ccc(-c3ccc4oc5ccc(-c6c7ccccc7c(-c7cc8ccccc8c8ccccc78)c7ccccc67)cc5c4c3)cc2-c2c1ccc1ccccc21 ; CC1(C)c2ccc(-c3ccc4oc5ccc(-c6c7ccccc7c(-c7cc8ccc9ccccc9c8c8ccccc78)c7ccccc67)cc5c4c3)cc2-c2c1ccc1ccccc21]

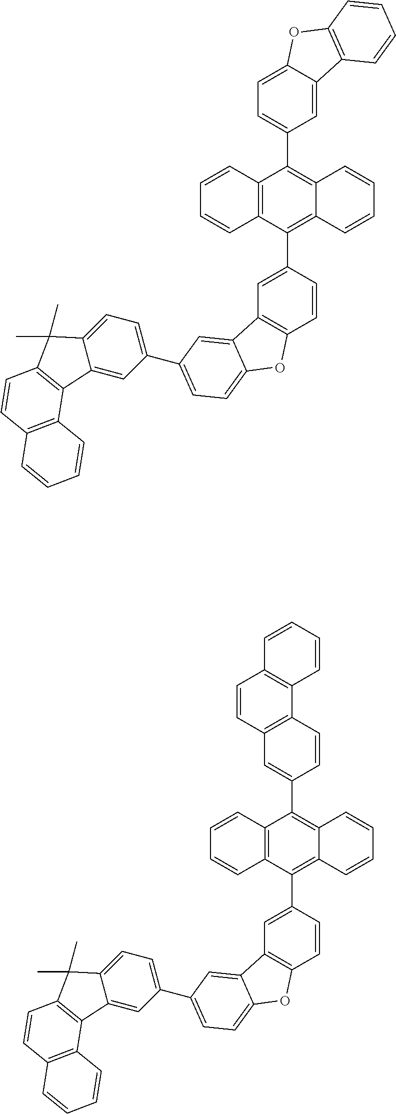 [SMILES: CC1(C)c2ccc(-c3ccc4oc5ccc(-c6c7ccccc7c(-c7ccc8oc9ccccc9c8c7)c7ccccc67)cc5c4c3)cc2-c2c1ccc1ccccc21 ; CC1(C)c2ccc(-c3ccc4oc5ccc(-c6c7ccccc7c(-c7ccc8c(ccc9ccccc98)c7)c7ccccc67)cc5c4c3)cc2-c2c1ccc1ccccc21]

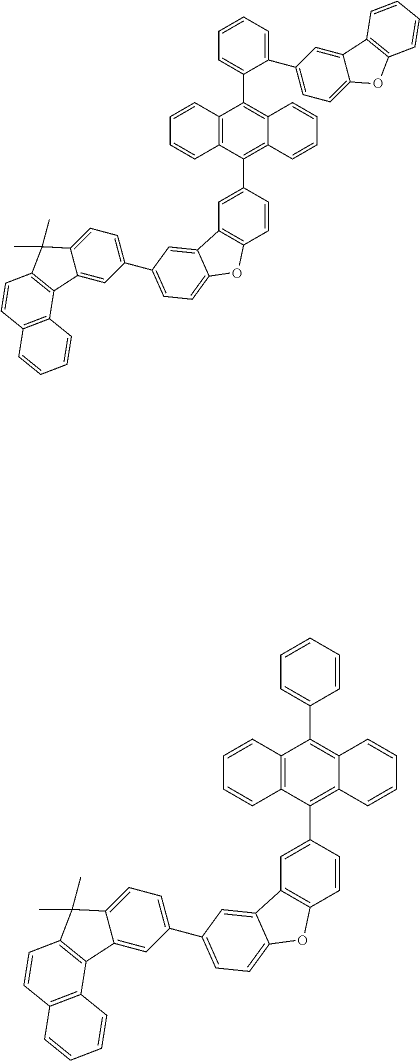 [SMILES: CC1(C)c2ccc(-c3ccc4oc5ccc(-c6c7ccccc7c(-c7ccccc7-c7ccc8oc9ccccc9c8c7)c7ccccc67)cc5c4c3)cc2-c2c1ccc1ccccc21 ; CC1(C)c2ccc(-c3ccc4oc5ccc(-c6c7ccccc7c(-c7ccccc7)c7ccccc67)cc5c4c3)cc2-c2c1ccc1ccccc21]

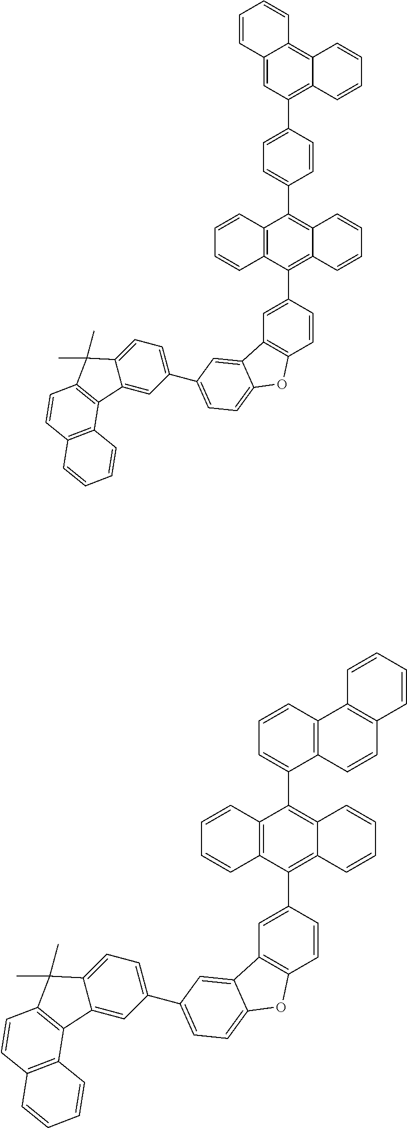 [SMILES: CC1(C)c2ccc(-c3ccc4oc5ccc(-c6c7ccccc7c(-c7ccc(-c8cc9ccccc9c9ccccc89)cc7)c7ccccc67)cc5c4c3)cc2-c2c1ccc1ccccc21 ; CC1(C)c2ccc(-c3ccc4oc5ccc(-c6c7ccccc7c(-c7cccc8c7ccc7ccccc78)c7ccccc67)cc5c4c3)cc2-c2c1ccc1ccccc21]

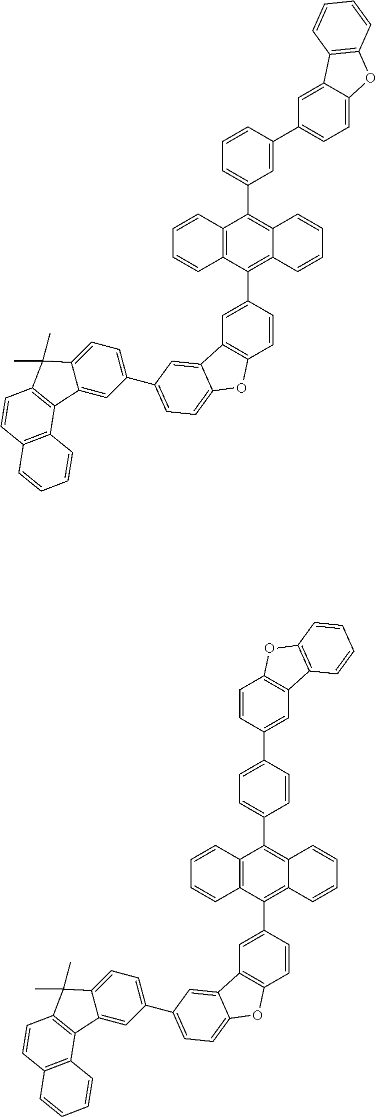 [SMILES: CC1(C)c2ccc(-c3ccc4oc5ccc(-c6c7ccccc7c(-c7cccc(-c8ccc9oc%10ccccc%10c9c8)c7)c7ccccc67)cc5c4c3)cc2-c2c1ccc1ccccc21 ; CC1(C)c2ccc(-c3ccc4oc5ccc(-c6c7ccccc7c(-c7ccc(-c8ccc9oc%10ccccc%10c9c8)cc7)c7ccccc67)cc5c4c3)cc2-c2c1ccc1ccccc21]

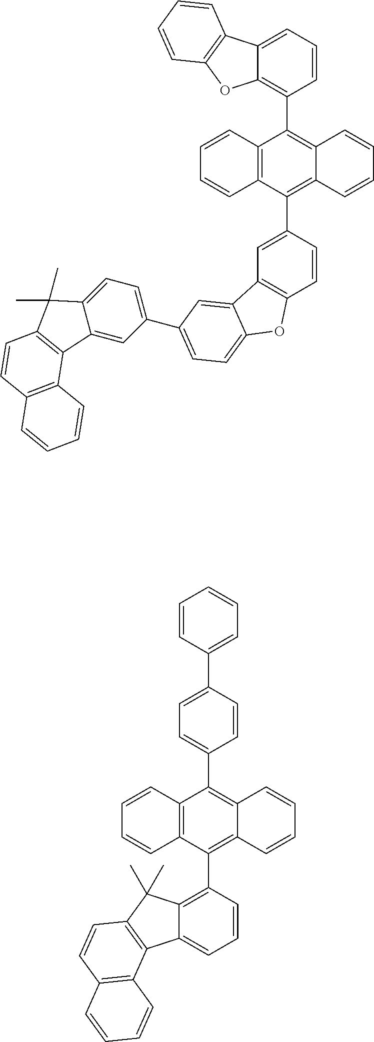

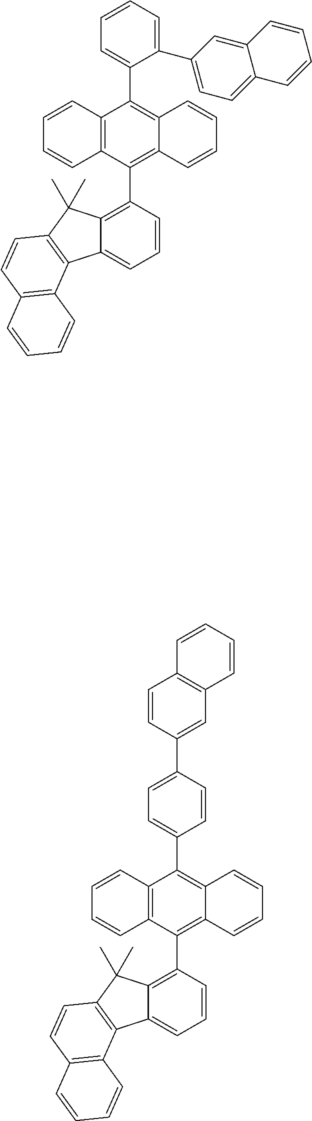

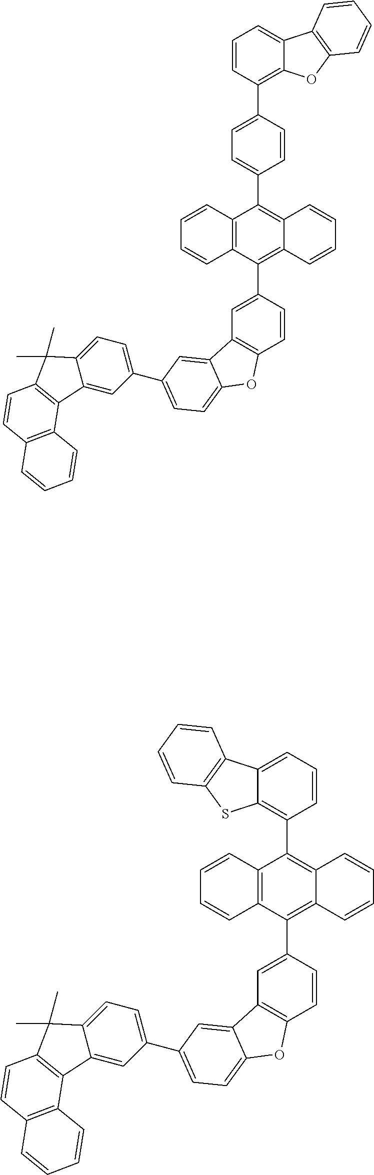 [SMILES: CC1(C)c2ccc(-c3ccc4oc5ccc(-c6c7ccccc7c(-c7ccc(-c8cccc9c8oc8ccccc89)cc7)c7ccccc67)cc5c4c3)cc2-c2c1ccc1ccccc21 ; CC1(C)c2ccc(-c3ccc4oc5ccc(-c6c7ccccc7c(-c7cccc8c7sc7ccccc78)c7ccccc67)cc5c4c3)cc2-c2c1ccc1ccccc21]

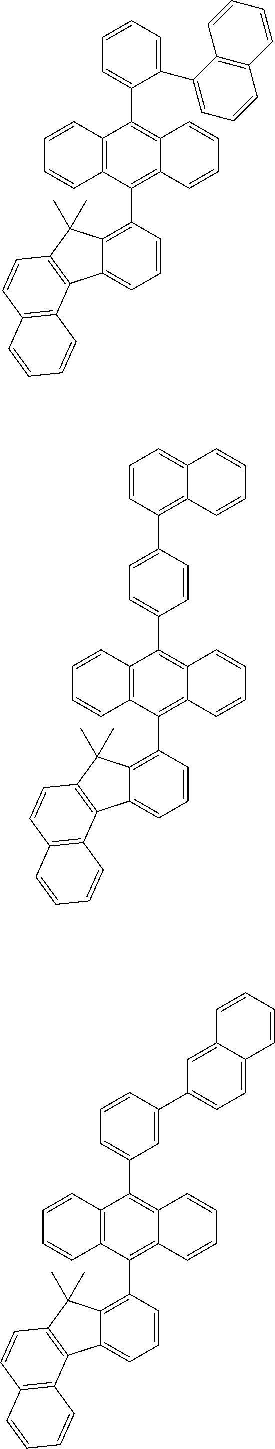

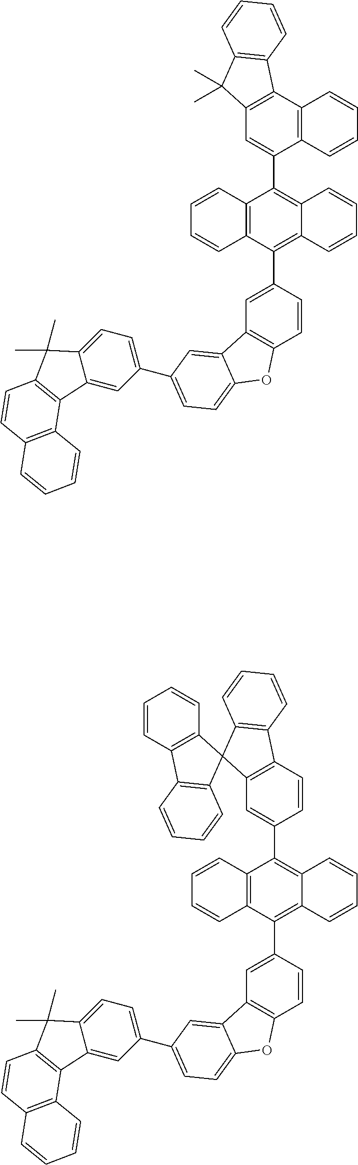 [SMILES: CC1(C)c2ccc(-c3ccc4oc5ccc(-c6c7ccccc7c(-c7cc8c(c9ccccc79)-c7ccccc7C8(C)C)c7ccccc67)cc5c4c3)cc2-c2c1ccc1ccccc21 ; CC1(C)c2ccc(-c3ccc4oc5ccc(-c6c7ccccc7c(-c7ccc8c(c7)C7(c9ccccc9-c9ccccc97)c7ccccc7-8)c7ccccc67)cc5c4c3)cc2-c2c1ccc1ccccc21]

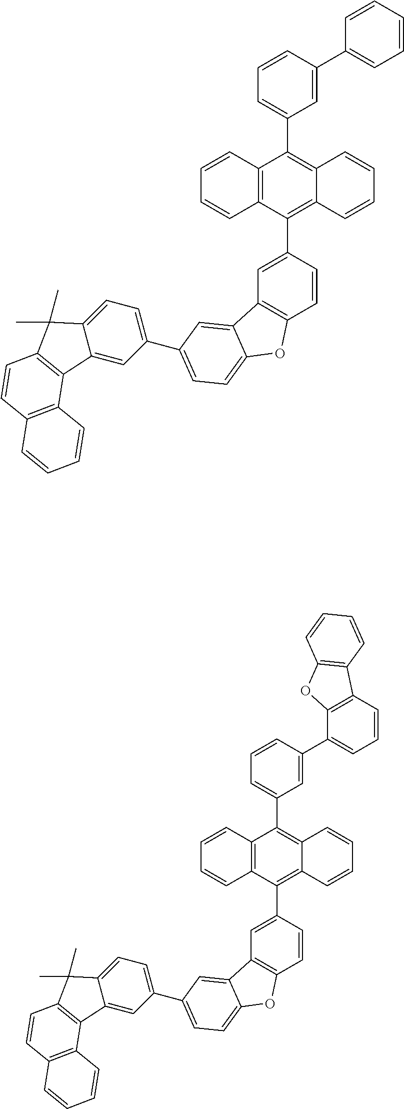 [SMILES: CC1(C)c2ccc(-c3ccc4oc5ccc(-c6c7ccccc7c(-c7cccc(-c8ccccc8)c7)c7ccccc67)cc5c4c3)cc2-c2c1ccc1ccccc21 ; CC1(C)c2ccc(-c3ccc4oc5ccc(-c6c7ccccc7c(-c7cccc(-c8cccc9c8oc8ccccc89)c7)c7ccccc67)cc5c4c3)cc2-c2c1ccc1ccccc21]

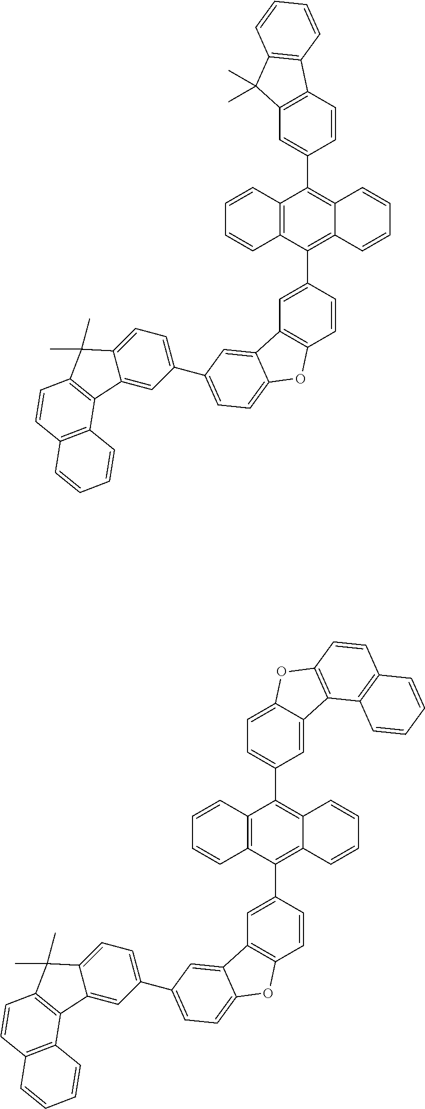 [SMILES: CC1(C)c2ccccc2-c2ccc(-c3c4ccccc4c(-c4ccc5oc6ccc(-c7ccc8c(c7)-c7c(ccc9ccccc79)C8(C)C)cc6c5c4)c4ccccc34)cc21 ; CC1(C)c2ccc(-c3ccc4oc5ccc(-c6c7ccccc7c(-c7ccc8oc9ccc%10ccccc%10c9c8c7)c7ccccc67)cc5c4c3)cc2-c2c1ccc1ccccc21]

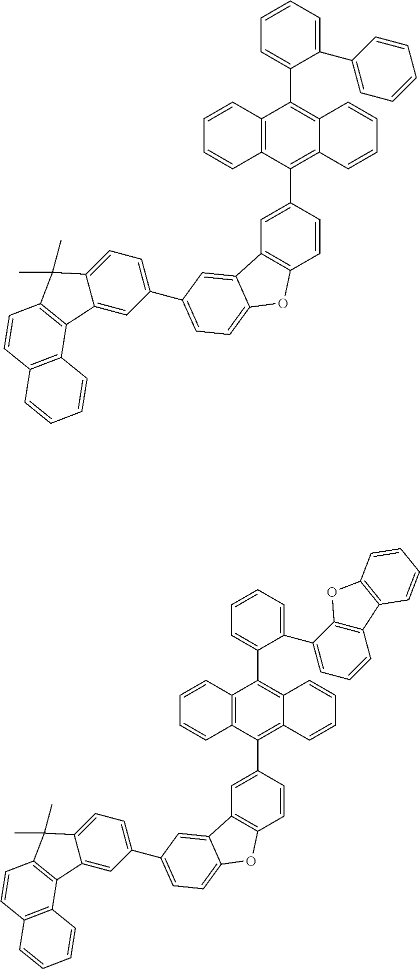 [SMILES: CC1(C)c2ccc(-c3ccc4oc5ccc(-c6c7ccccc7c(-c7ccccc7-c7ccccc7)c7ccccc67)cc5c4c3)cc2-c2c1ccc1ccccc21 ; CC1(C)c2ccc(-c3ccc4oc5ccc(-c6c7ccccc7c(-c7ccccc7-c7cccc8c7oc7ccccc78)c7ccccc67)cc5c4c3)cc2-c2c1ccc1ccccc21]

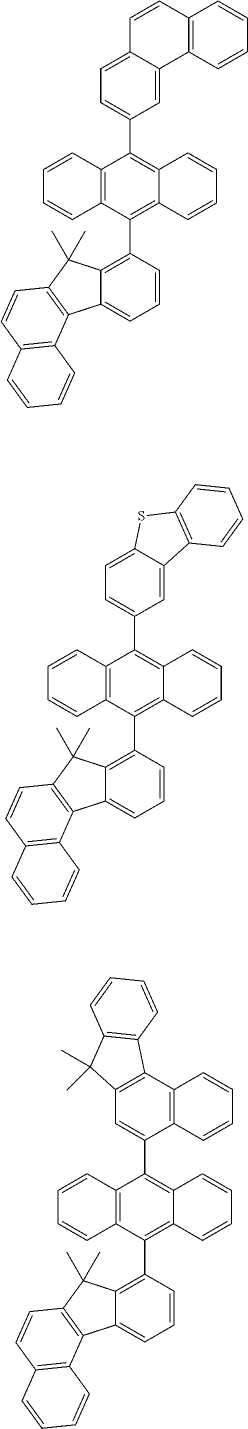

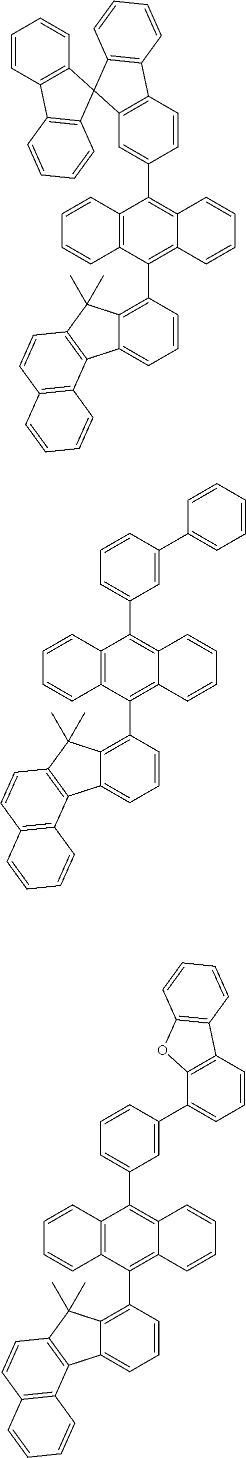

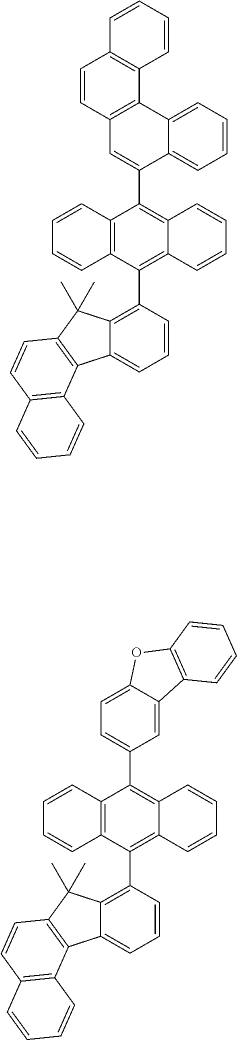

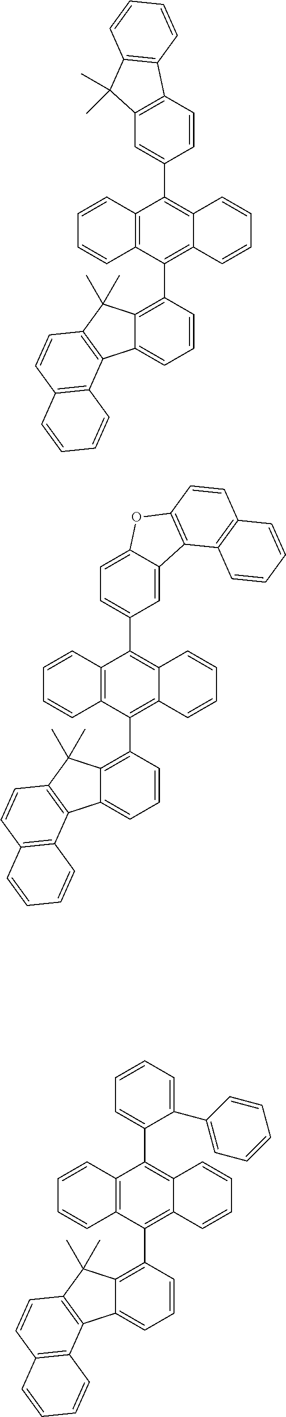

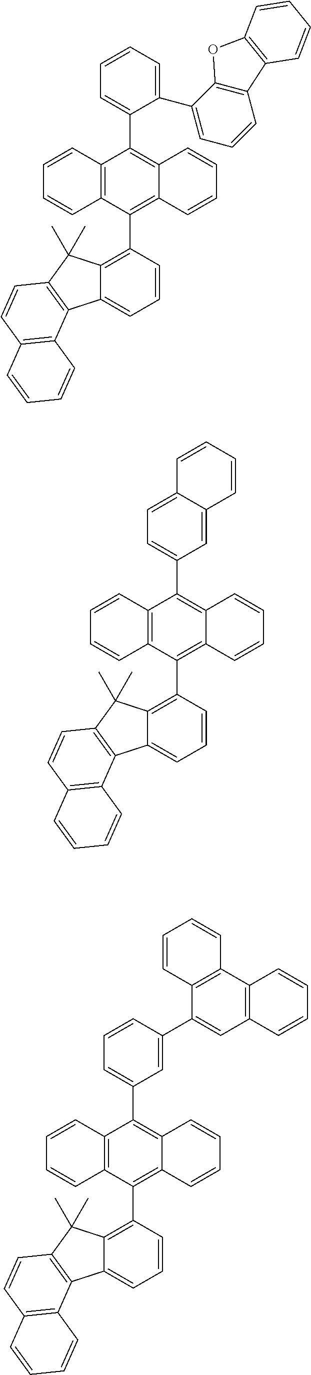

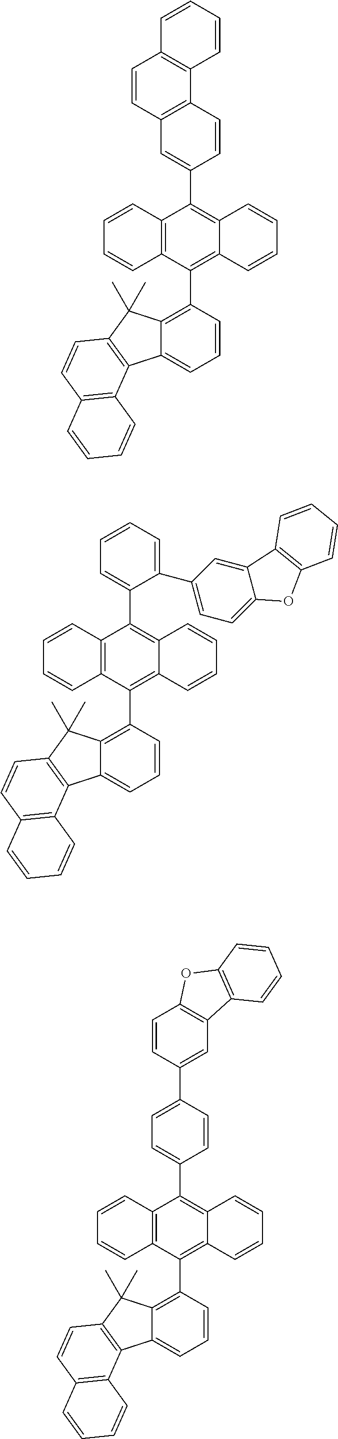

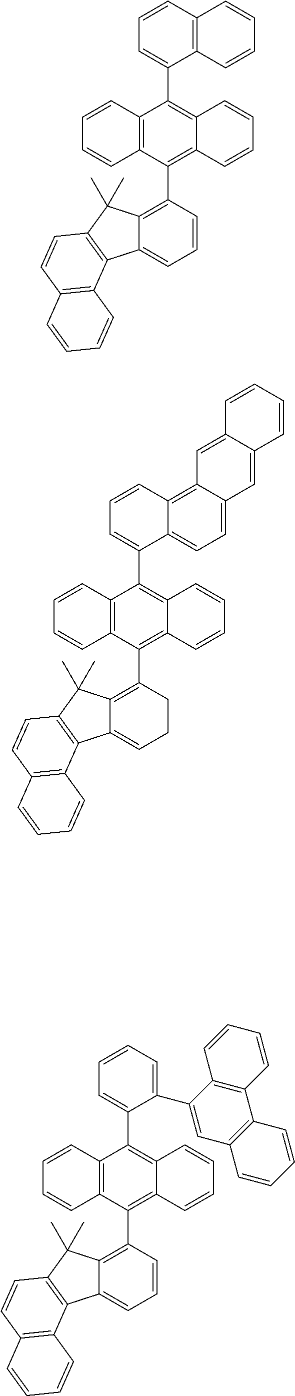

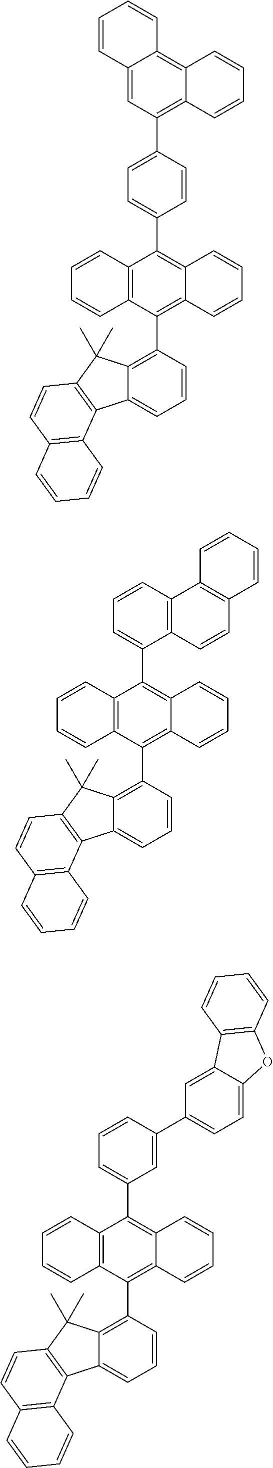

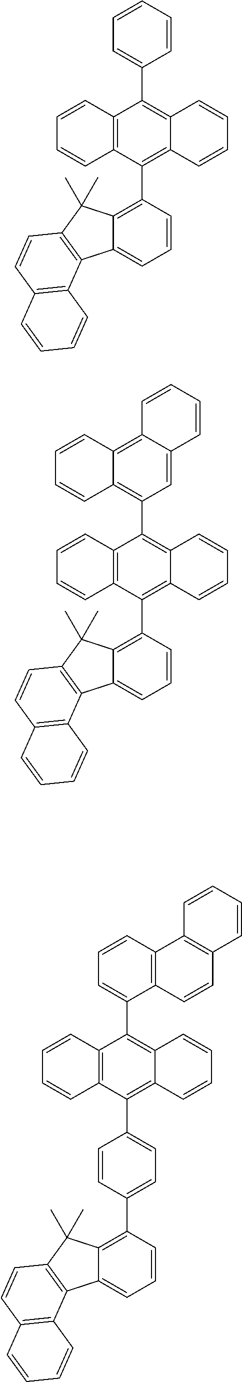

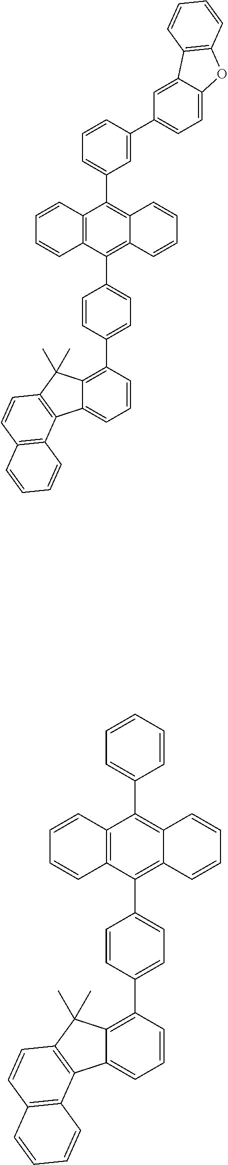

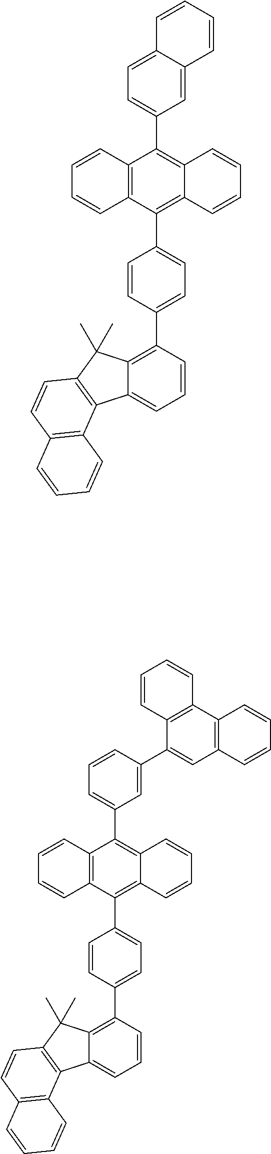

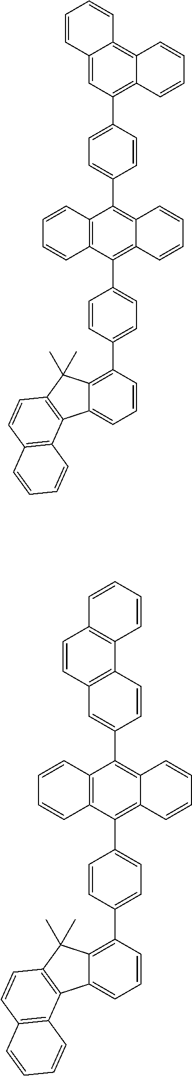

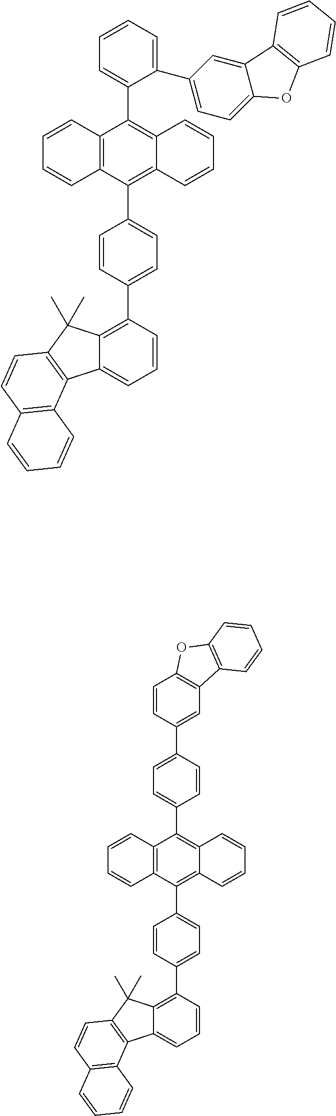 [SMILES: CC1(C)c2ccc3ccccc3c2-c2cccc(-c3ccc(-c4c5ccccc5c(-c5ccccc5-c5ccc6oc7ccccc7c6c5)c5ccccc45)cc3)c21 ; CC1(C)c2ccc3ccccc3c2-c2cccc(-c3ccc(-c4c5ccccc5c(-c5ccc(-c6ccc7oc8ccccc8c7c6)cc5)c5ccccc45)cc3)c21]

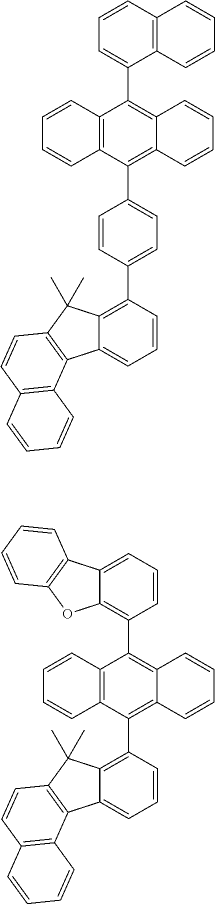

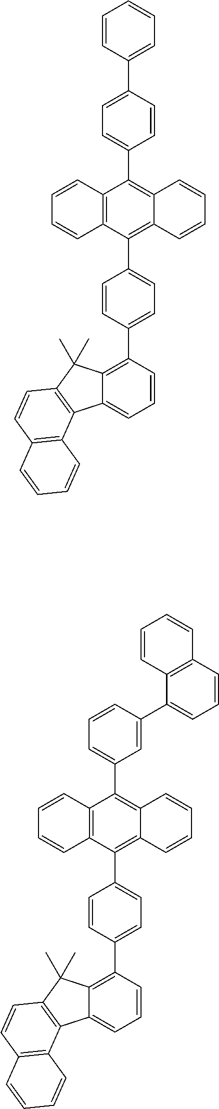

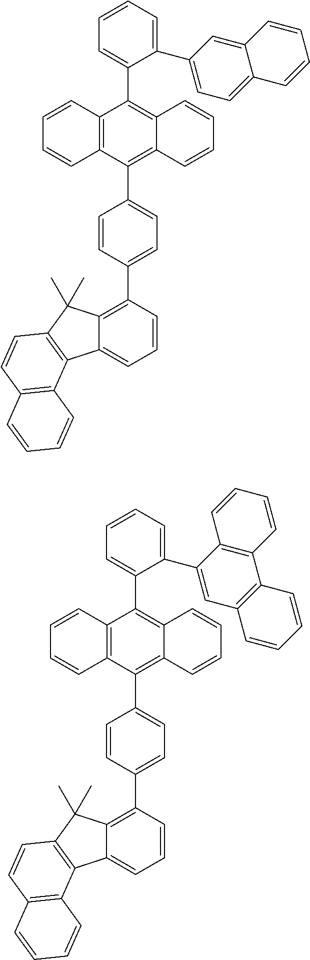

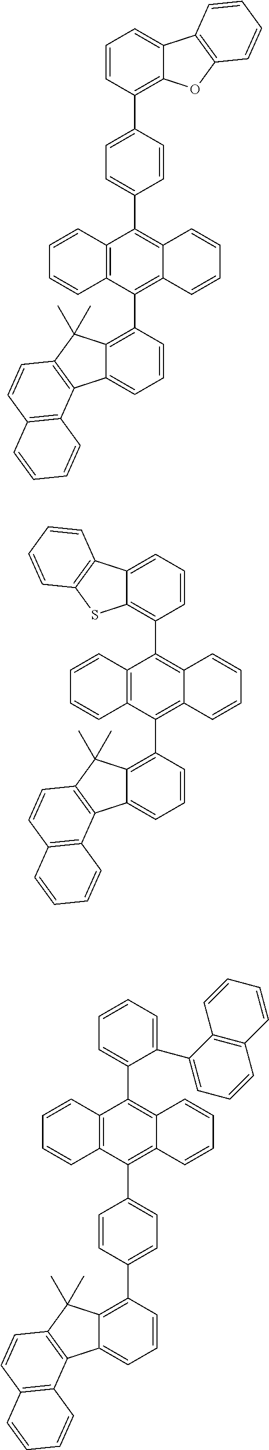

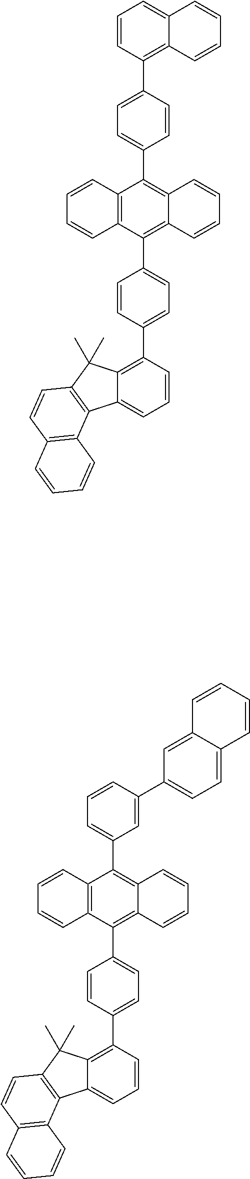

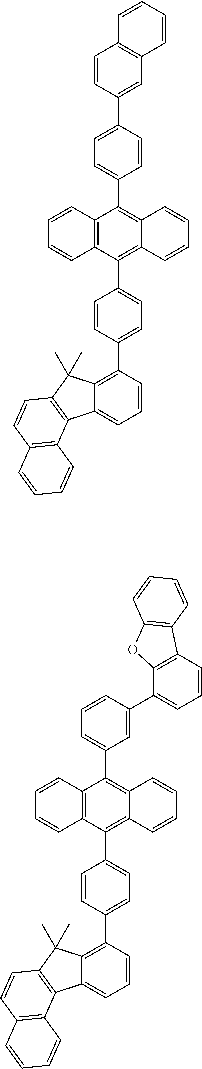

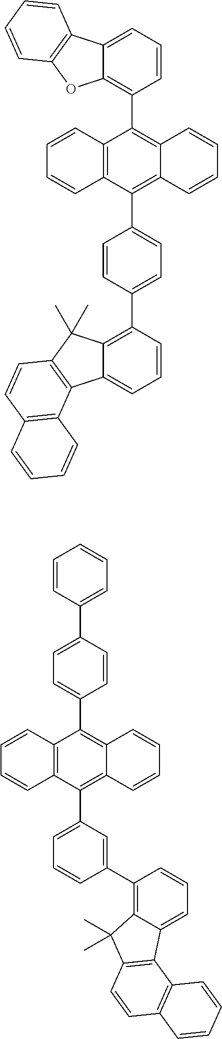

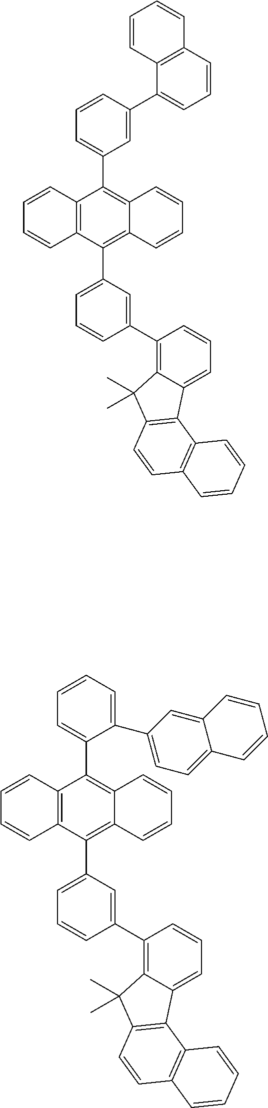

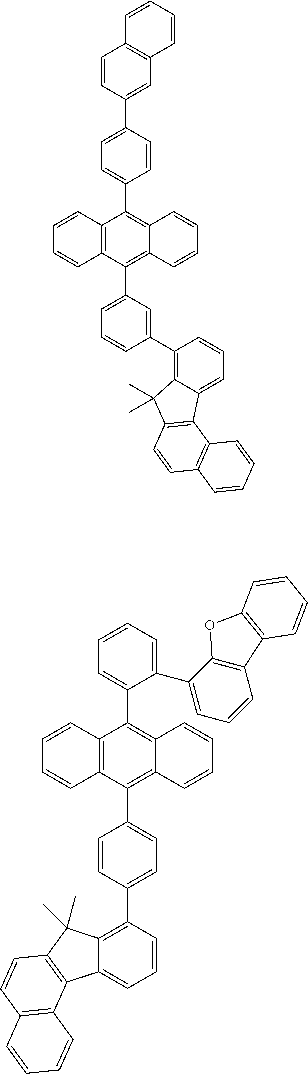

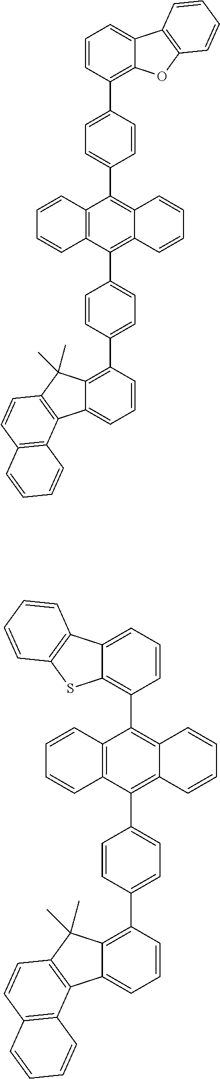 [SMILES: CC1(C)c2ccc3ccccc3c2-c2cccc(-c3ccc(-c4c5ccccc5c(-c5ccc(-c6cccc7c6oc6ccccc67)cc5)c5ccccc45)cc3)c21 ; CC1(C)c2ccc3ccccc3c2-c2cccc(-c3ccc(-c4c5ccccc5c(-c5cccc6c5sc5ccccc56)c5ccccc45)cc3)c21]

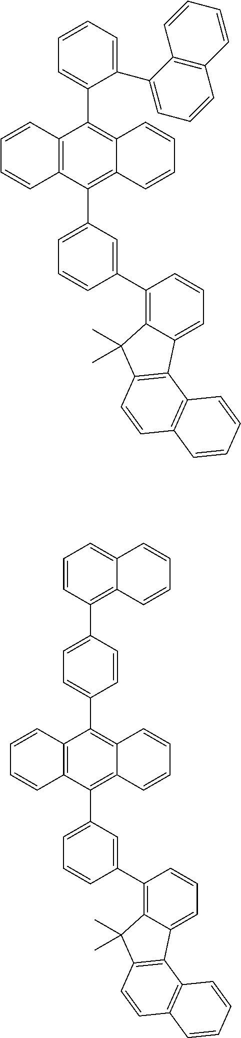

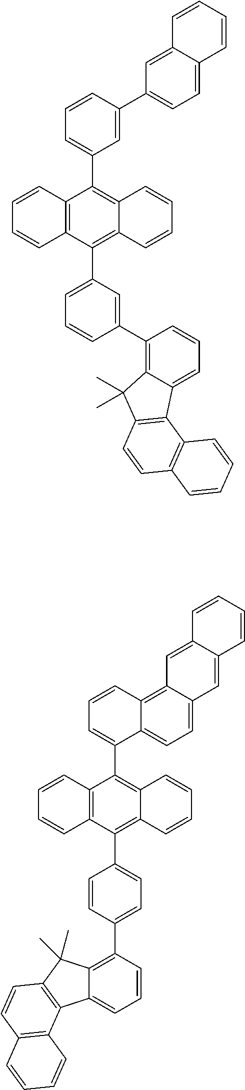

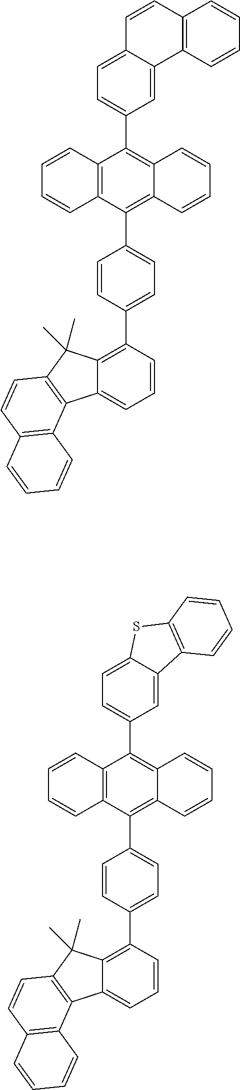

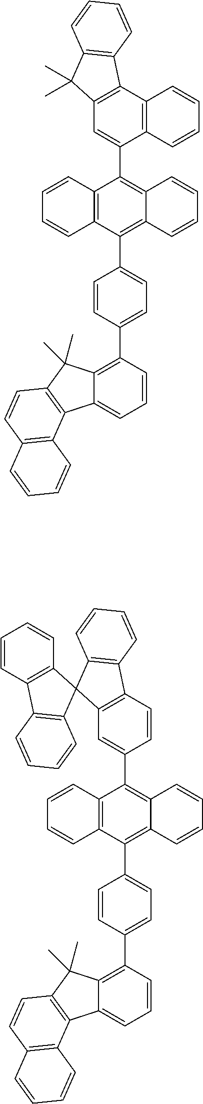

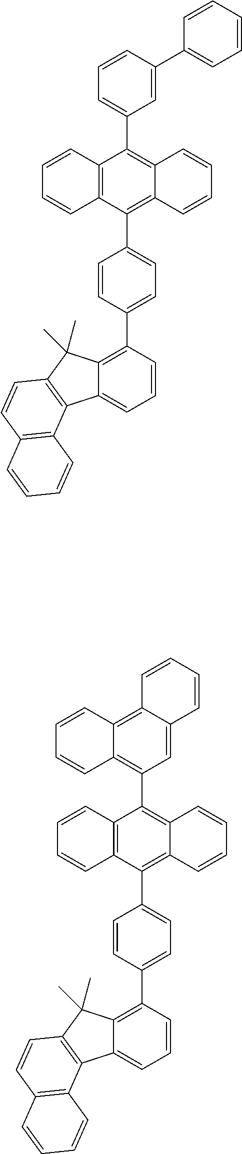

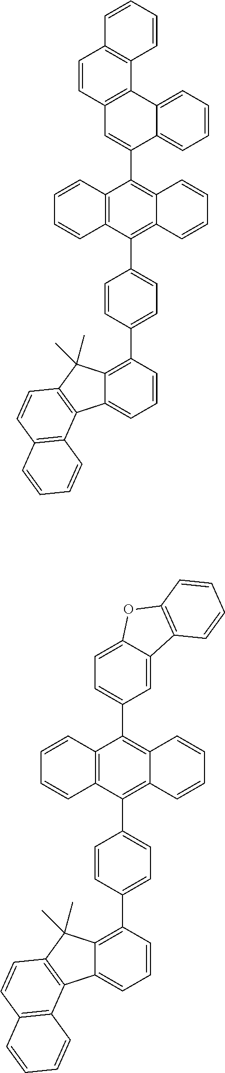

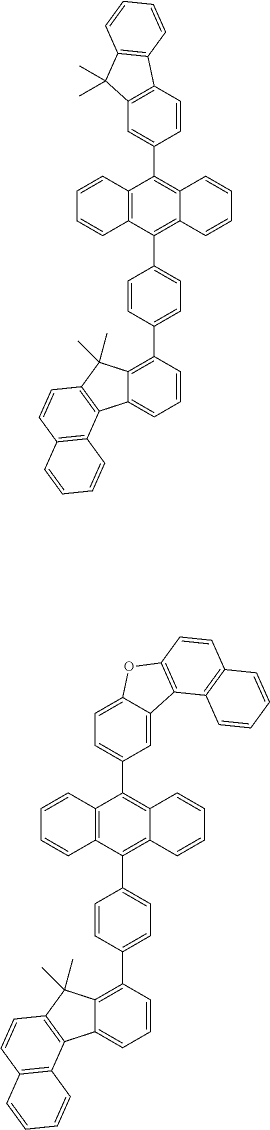

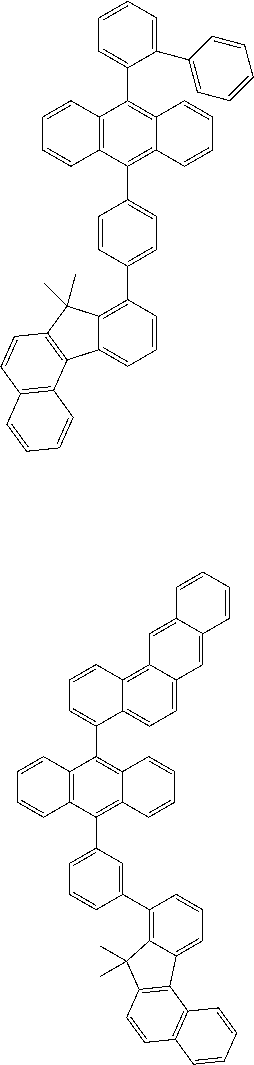

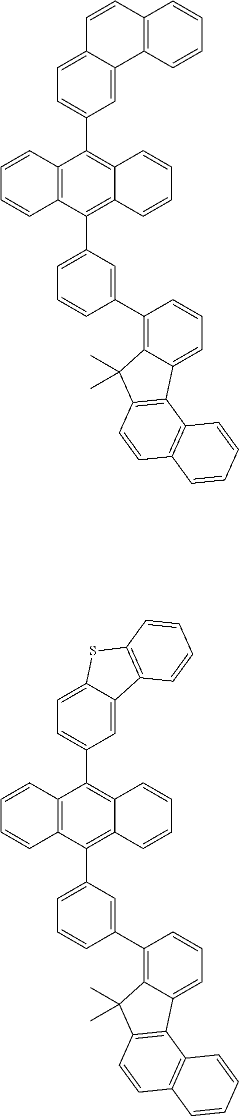

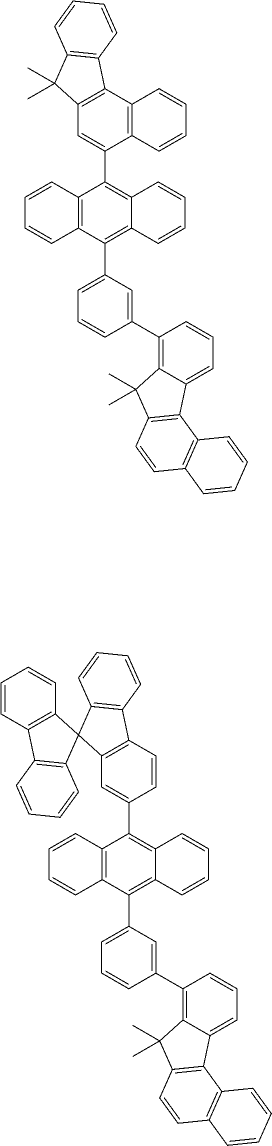

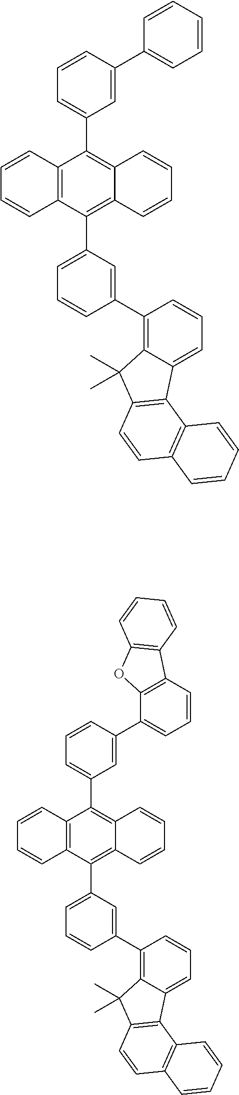

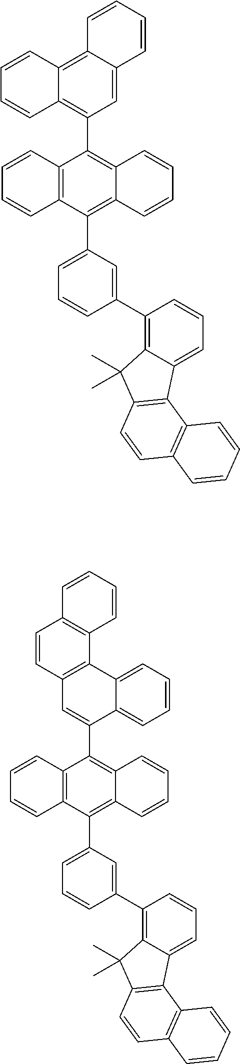

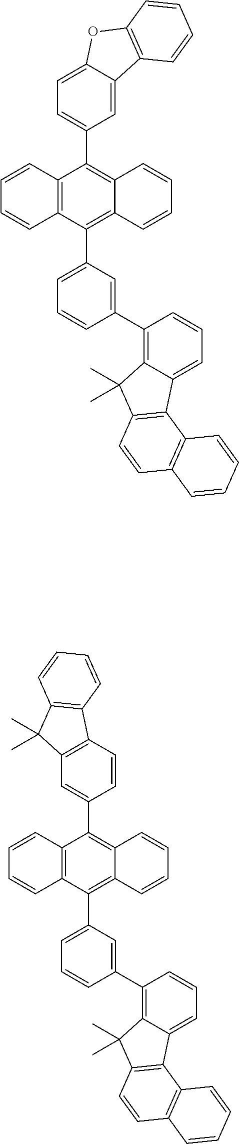

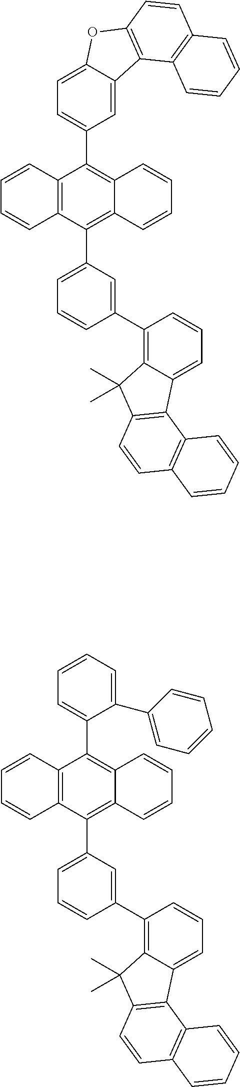

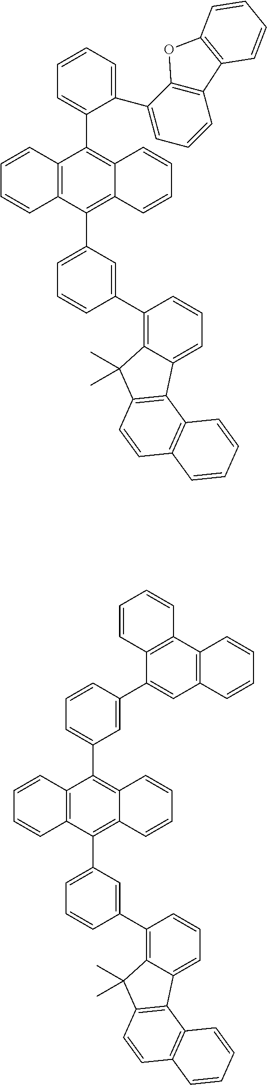

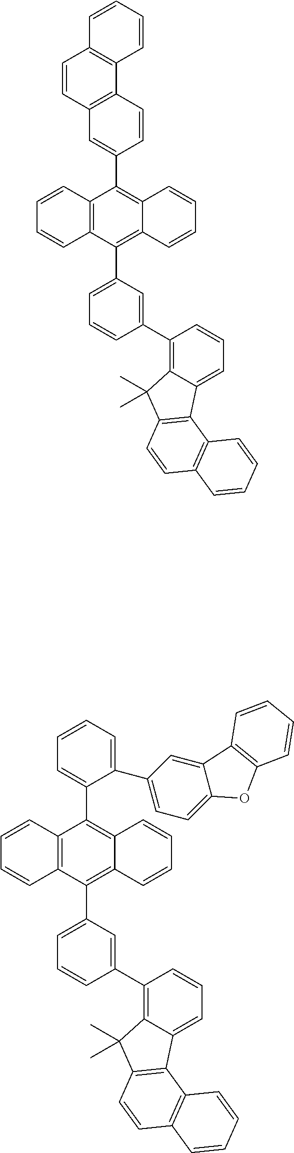

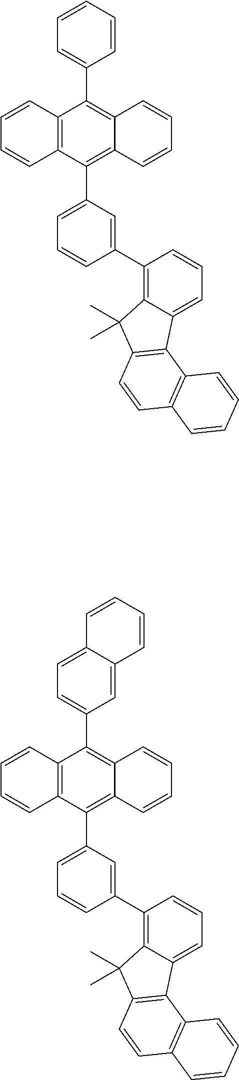

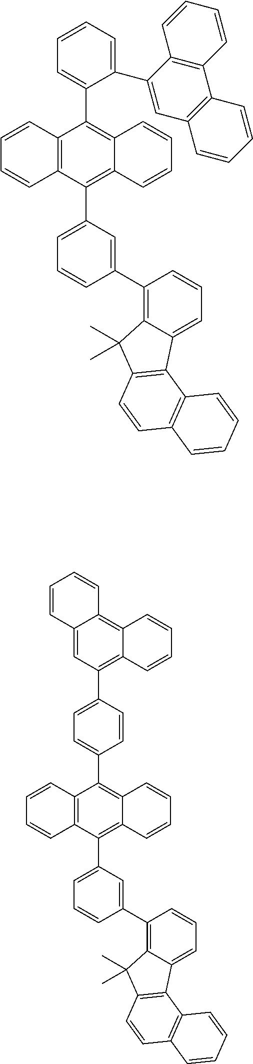

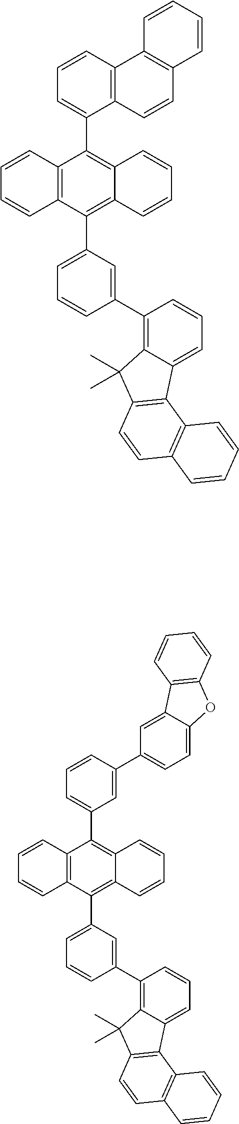

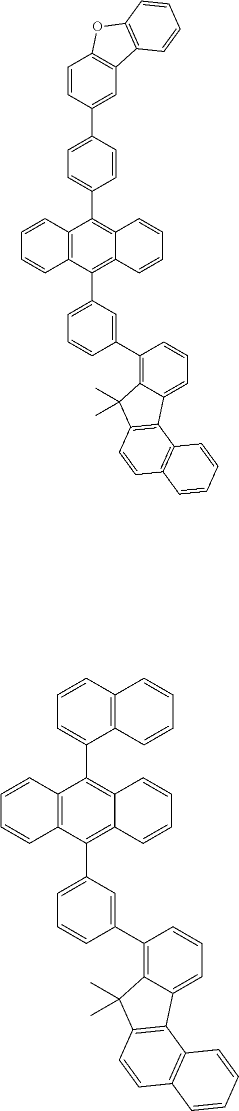

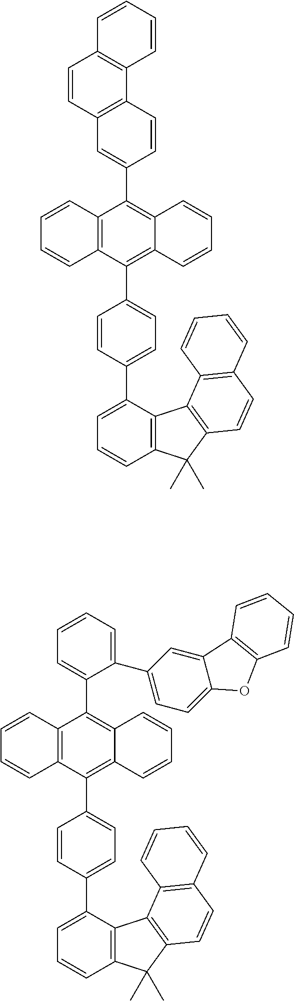

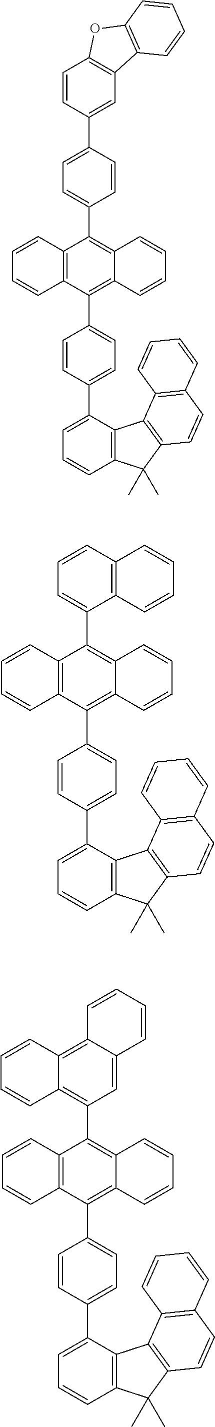

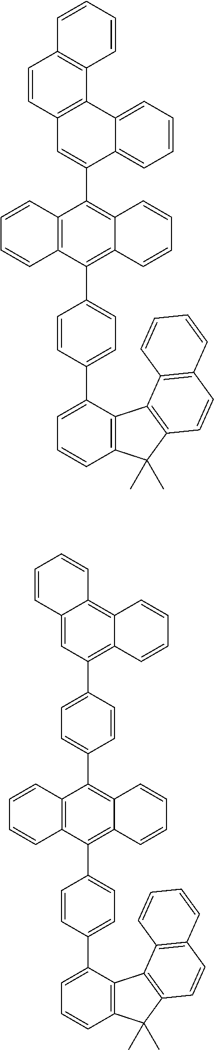

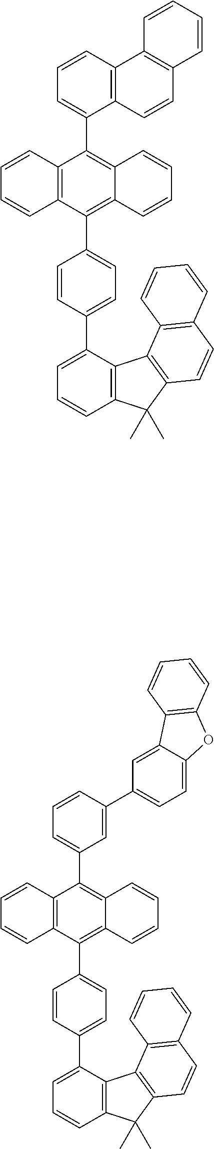

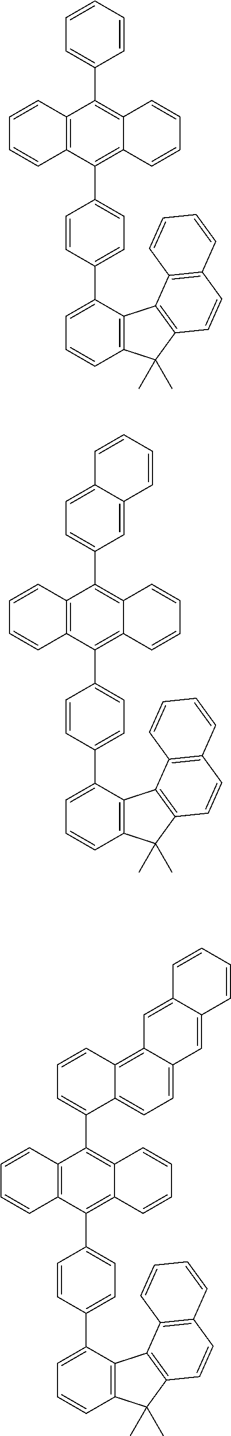

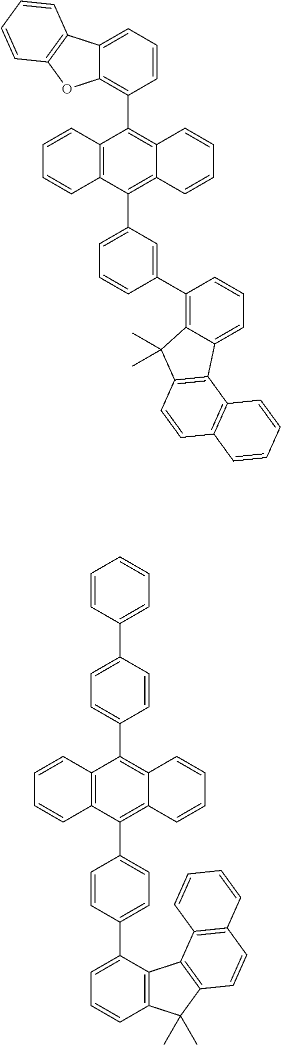

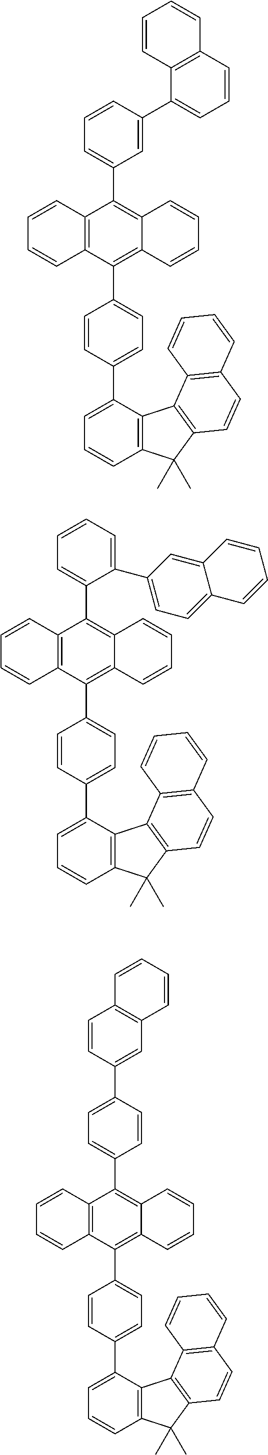

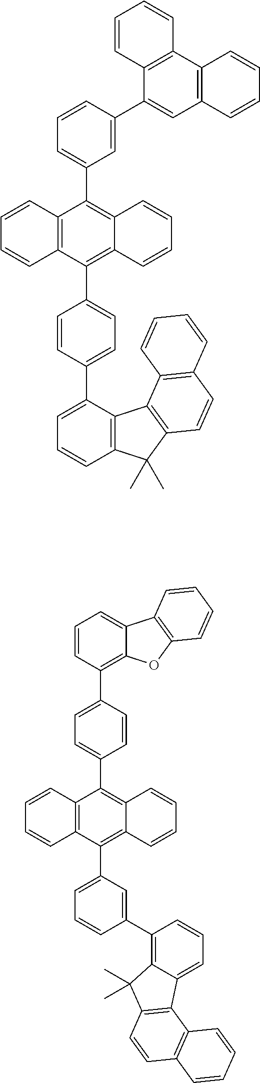

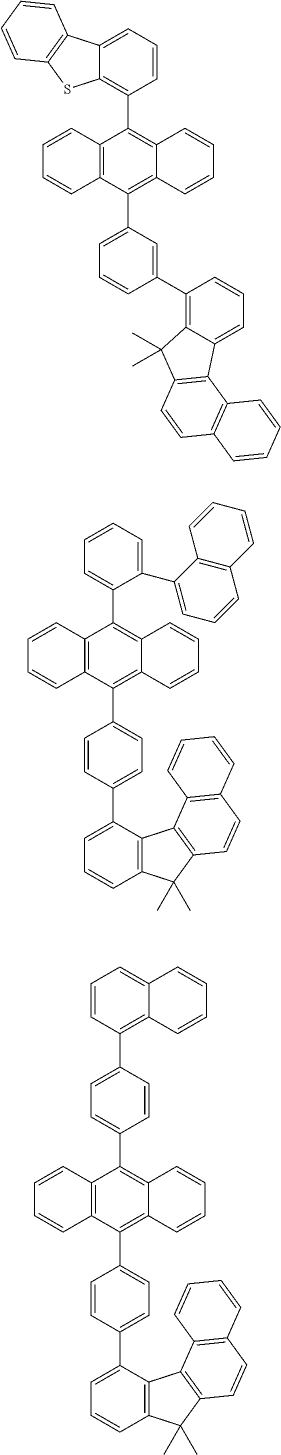

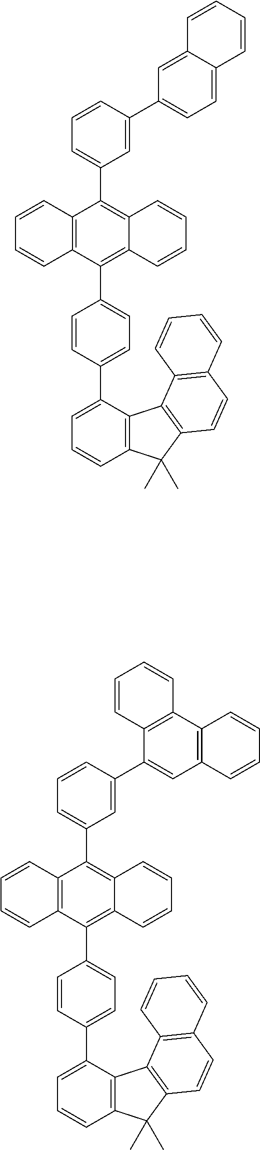

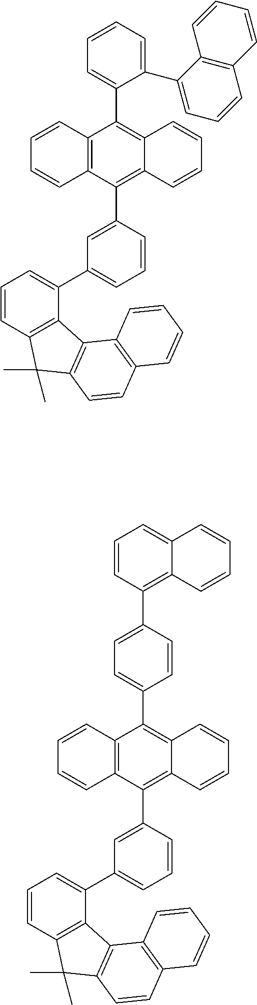

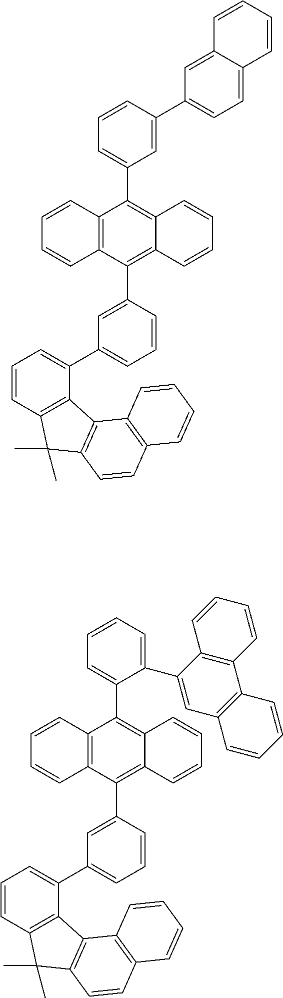

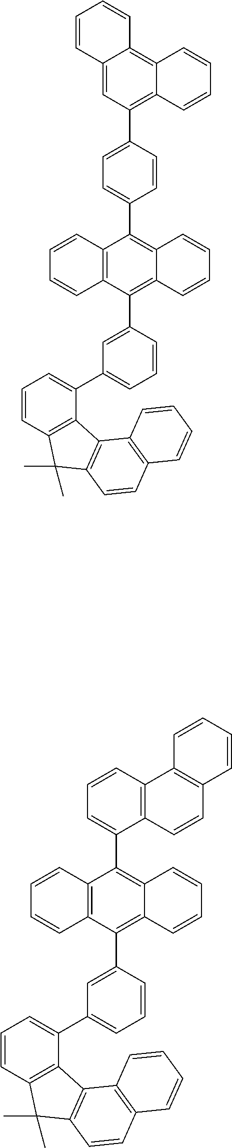

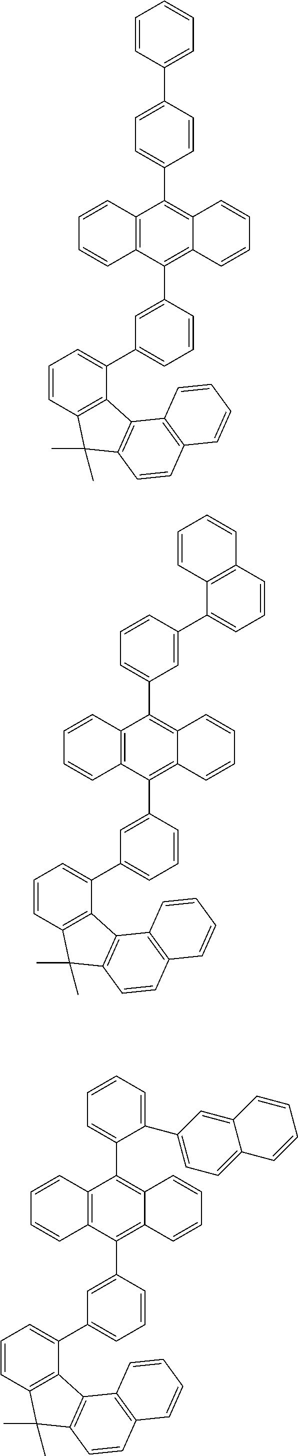

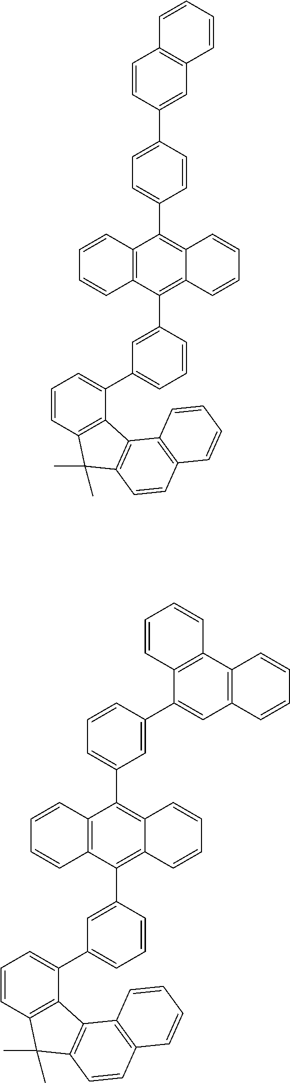

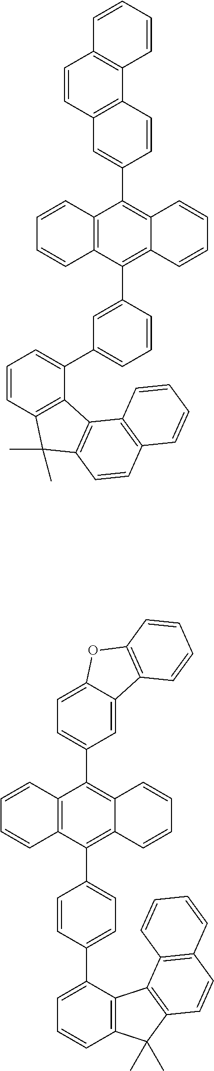

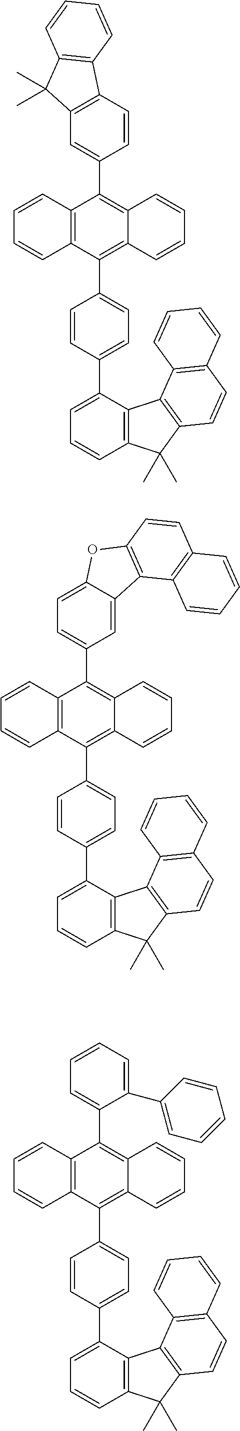

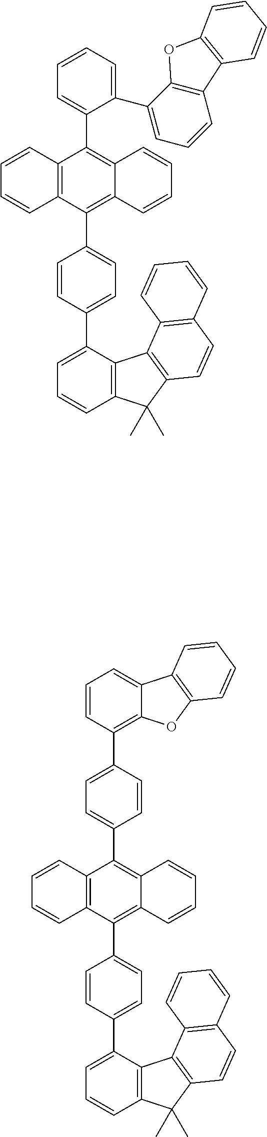 [SMILES: CC1(C)c2cccc(-c3ccc(-c4c5ccccc5c(-c5ccccc5-c5cccc6c5oc5ccccc56)c5ccccc45)cc3)c2-c2c1ccc1ccccc21 ; CC1(C)c2cccc(-c3ccc(-c4c5ccccc5c(-c5ccc(-c6cccc7c6oc6ccccc67)cc5)c5ccccc45)cc3)c2-c2c1ccc1ccccc21]

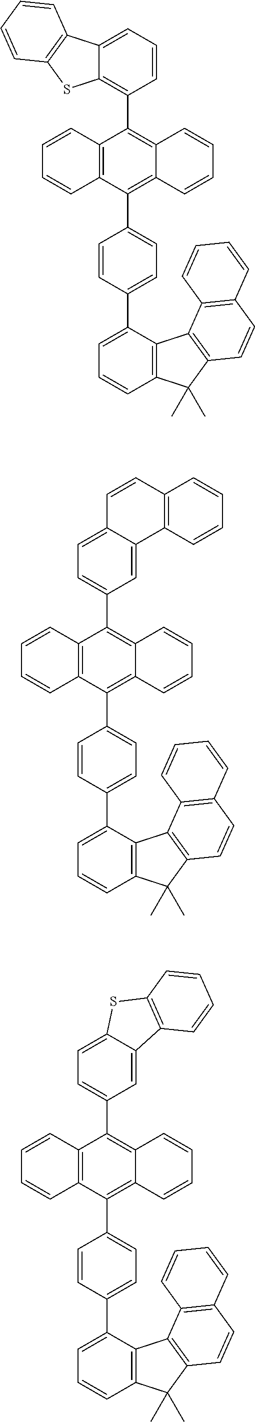

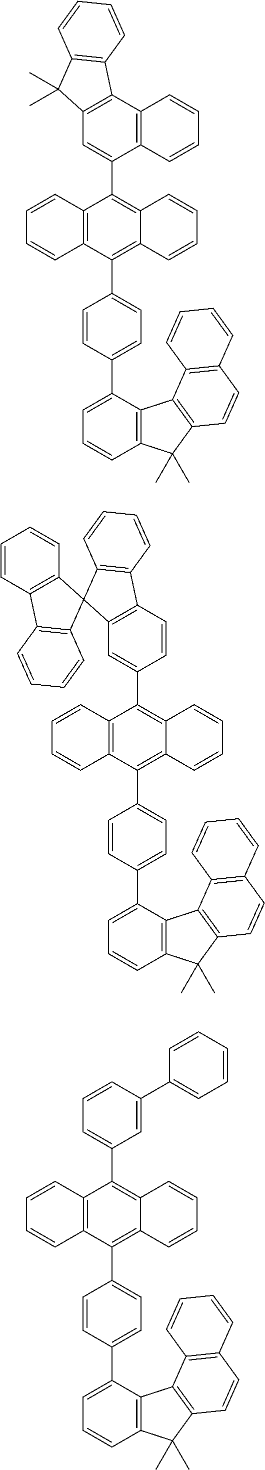

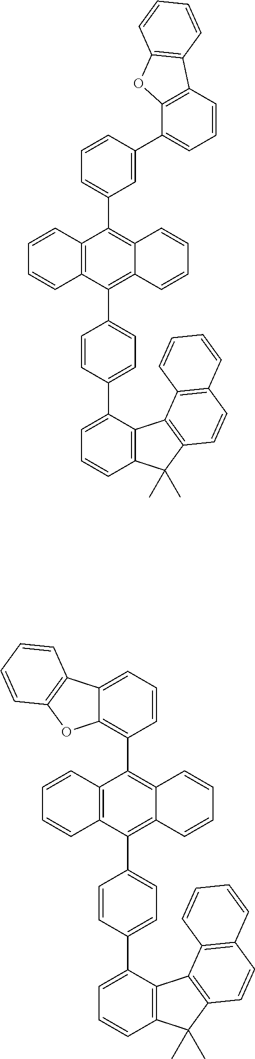 [SMILES: CC1(C)c2cccc(-c3ccc(-c4c5ccccc5c(-c5cccc(-c6cccc7c6oc6ccccc67)c5)c5ccccc45)cc3)c2-c2c1ccc1ccccc21 ; CC1(C)c2cccc(-c3ccc(-c4c5ccccc5c(-c5cccc6c5oc5ccccc56)c5ccccc45)cc3)c2-c2c1ccc1ccccc21]

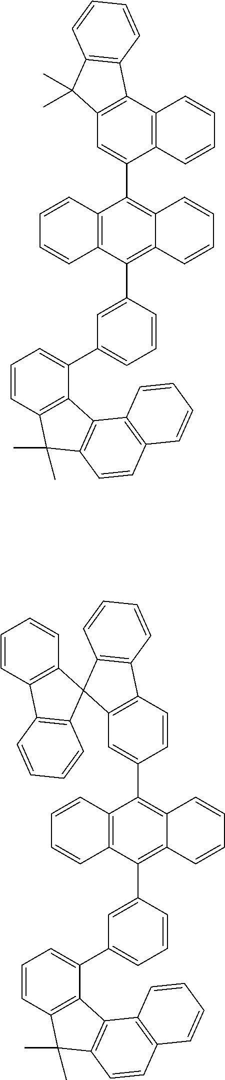

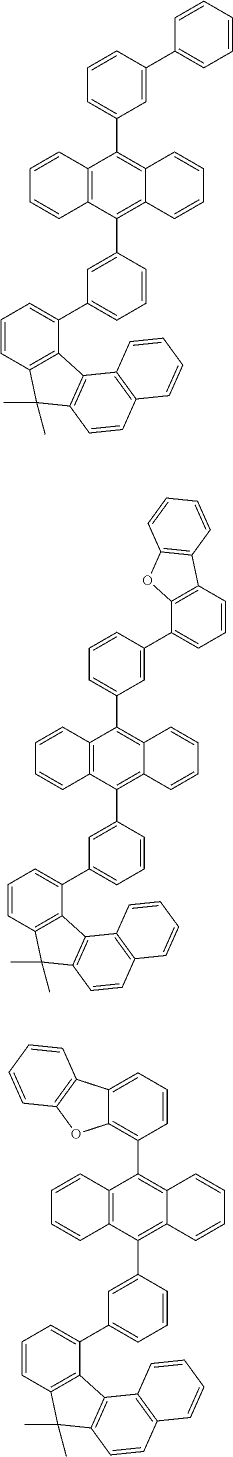

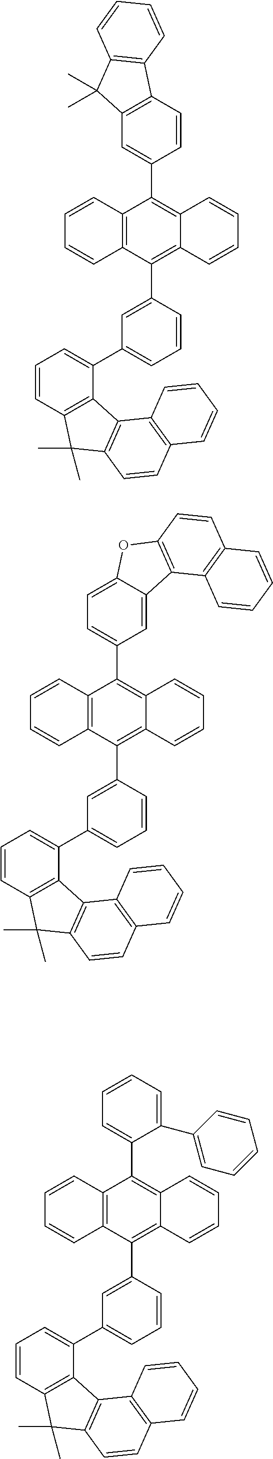

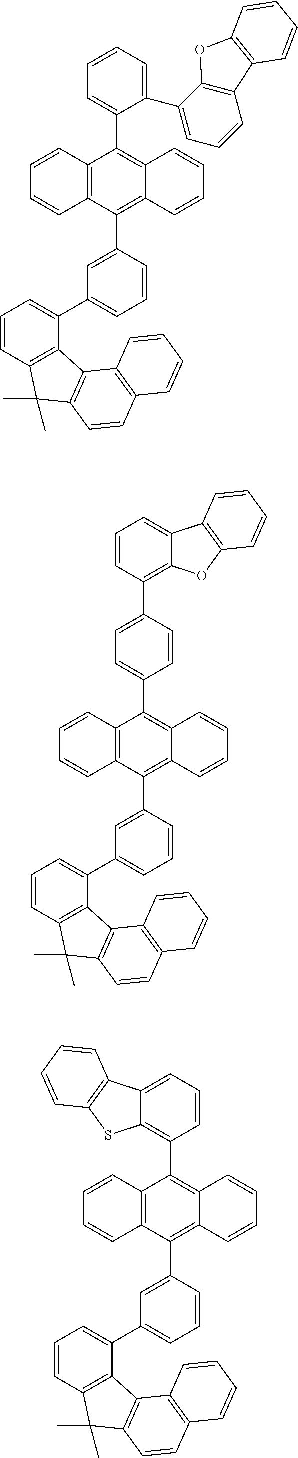 [SMILES: CC1(C)c2cccc(-c3cccc(-c4c5ccccc5c(-c5ccccc5-c5cccc6c5oc5ccccc56)c5ccccc45)c3)c2-c2c1ccc1ccccc21 ; CC1(C)c2cccc(-c3cccc(-c4c5ccccc5c(-c5ccc(-c6cccc7c6oc6ccccc67)cc5)c5ccccc45)c3)c2-c2c1ccc1ccccc21 ; CC1(C)c2cccc(-c3cccc(-c4c5ccccc5c(-c5cccc6c5sc5ccccc56)c5ccccc45)c3)c2-c2c1ccc1ccccc21]

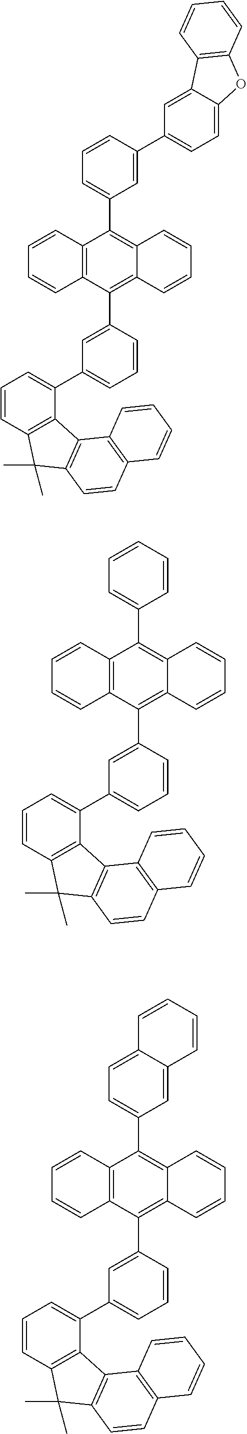

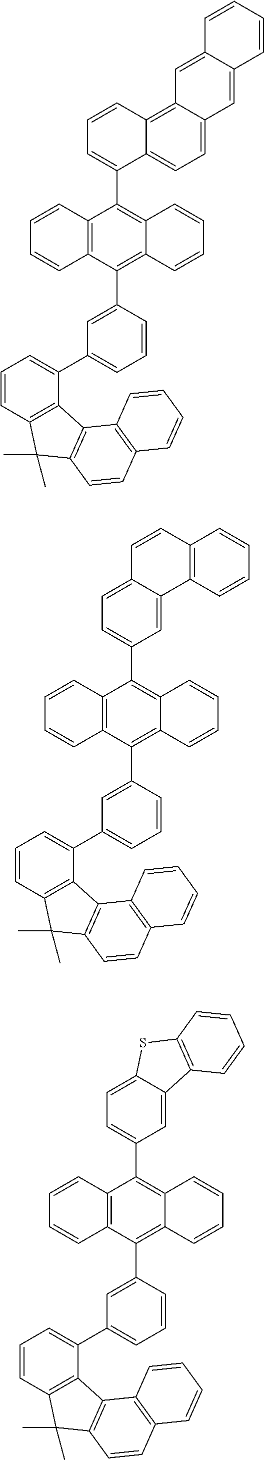

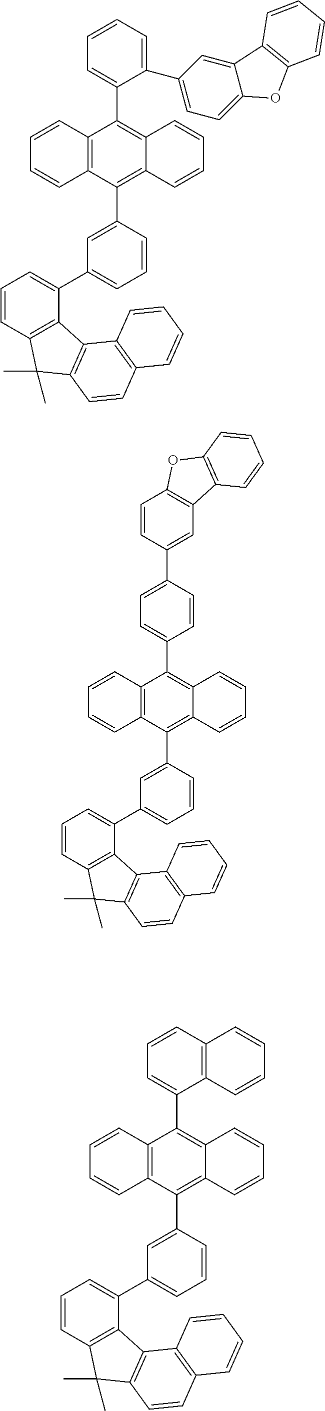

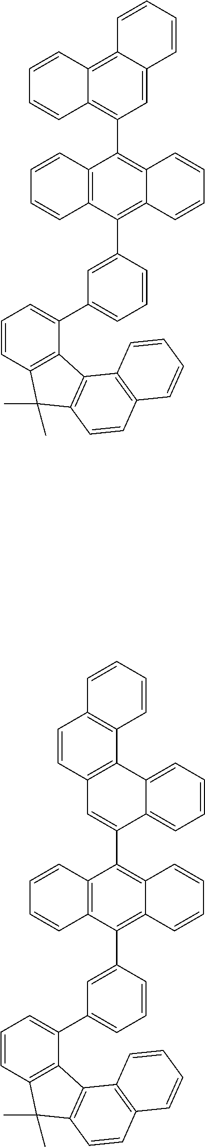

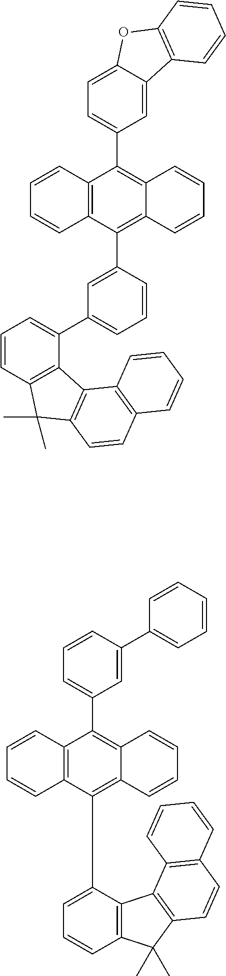

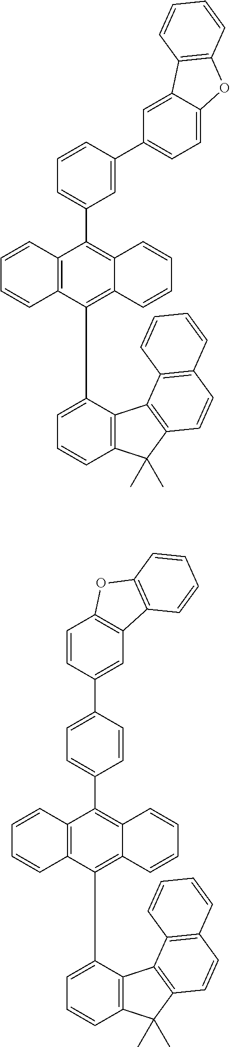 [SMILES: CC1(C)c2cccc(-c3c4ccccc4c(-c4cccc(-c5ccc6oc7ccccc7c6c5)c4)c4ccccc34)c2-c2c1ccc1ccccc21 ; CC1(C)c2cccc(-c3c4ccccc4c(-c4ccc(-c5ccc6oc7ccccc7c6c5)cc4)c4ccccc34)c2-c2c1ccc1ccccc21]

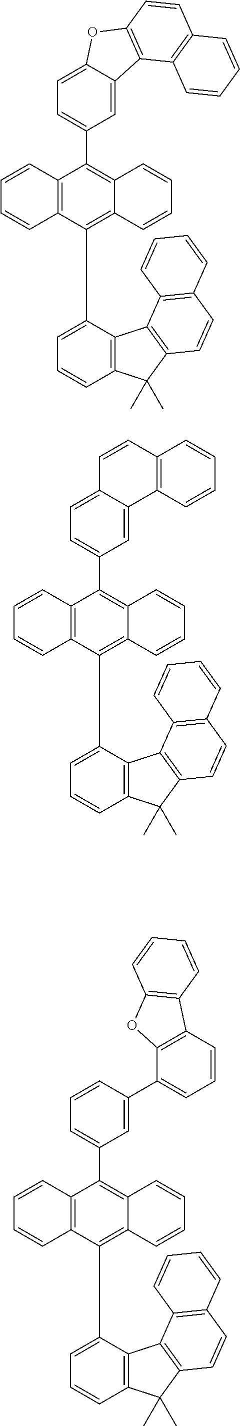

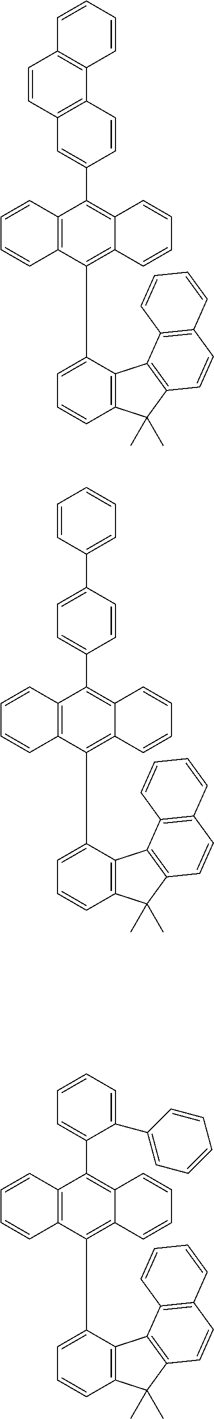

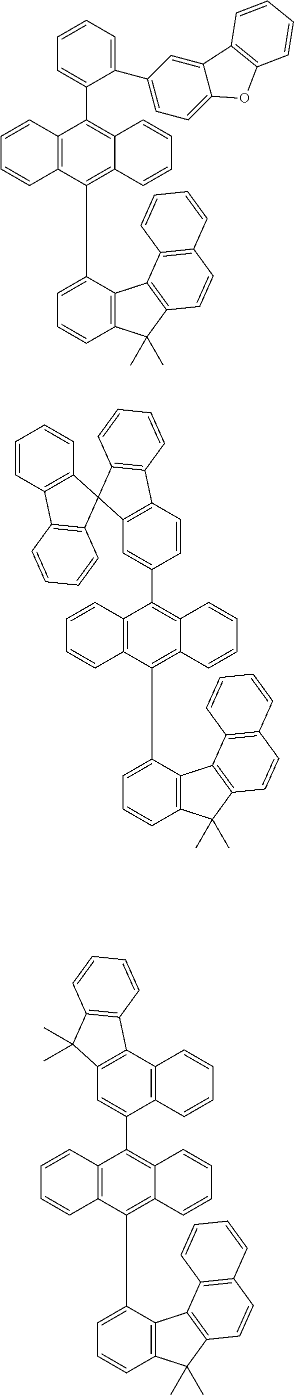

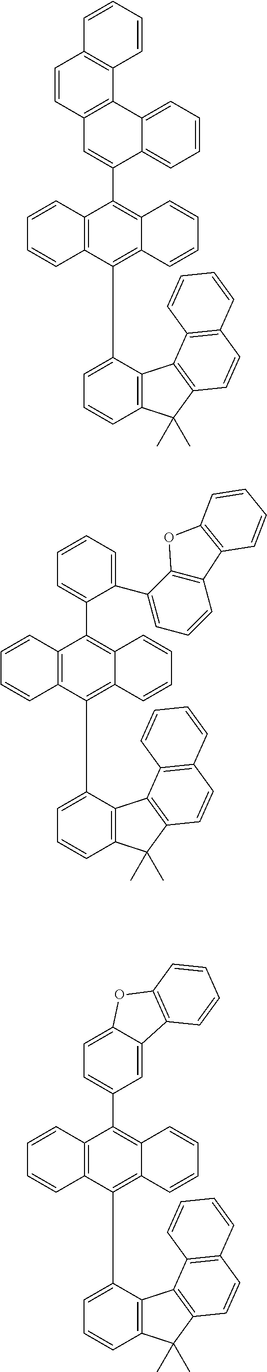

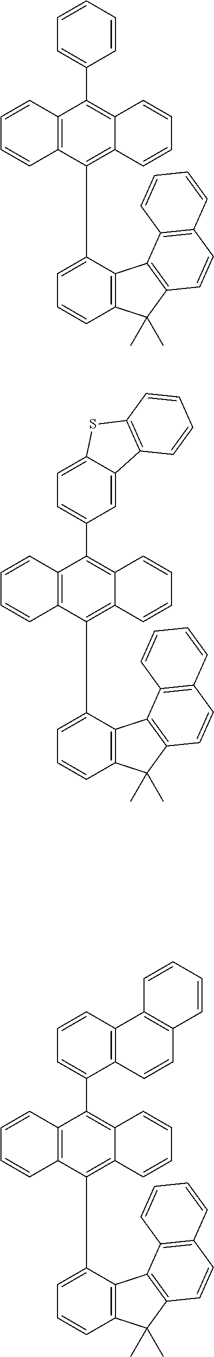

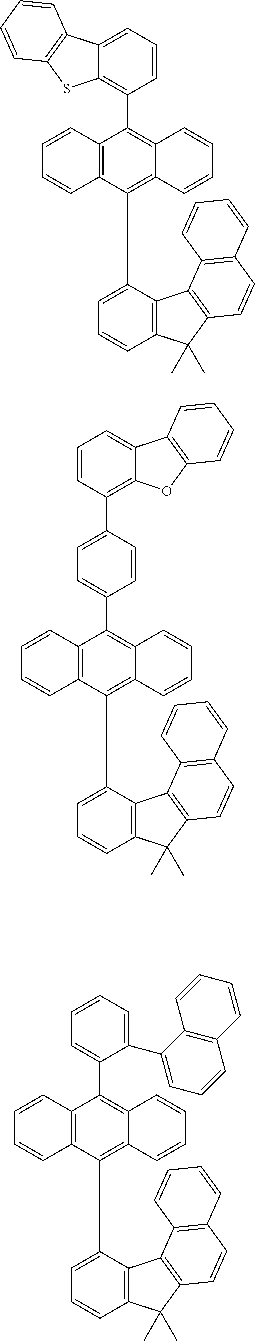

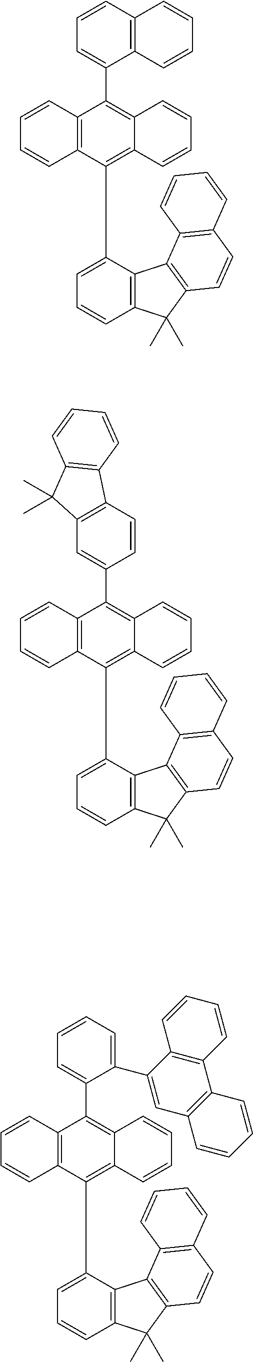

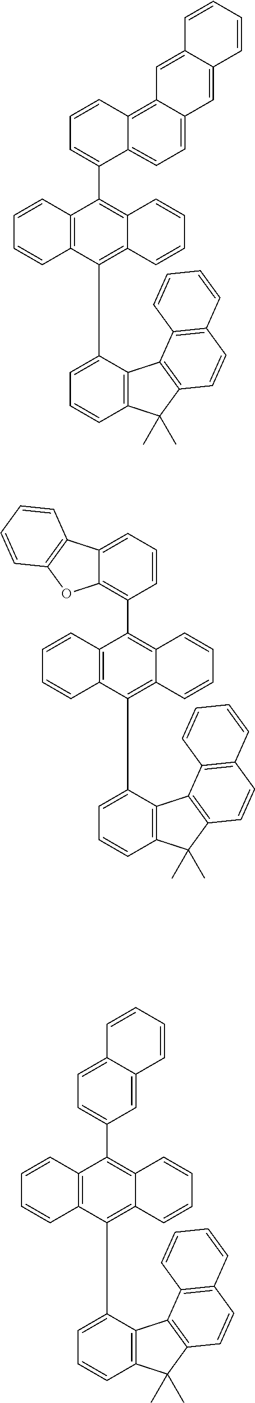

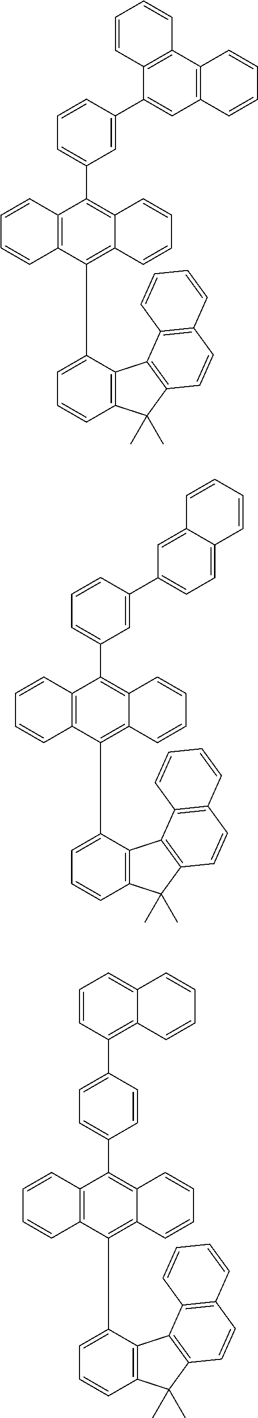

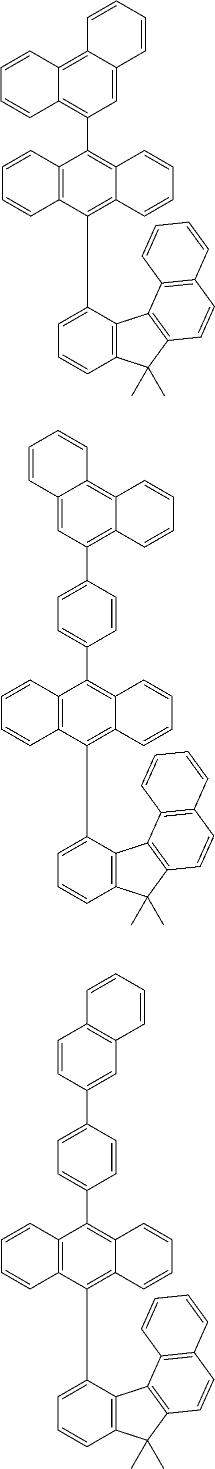

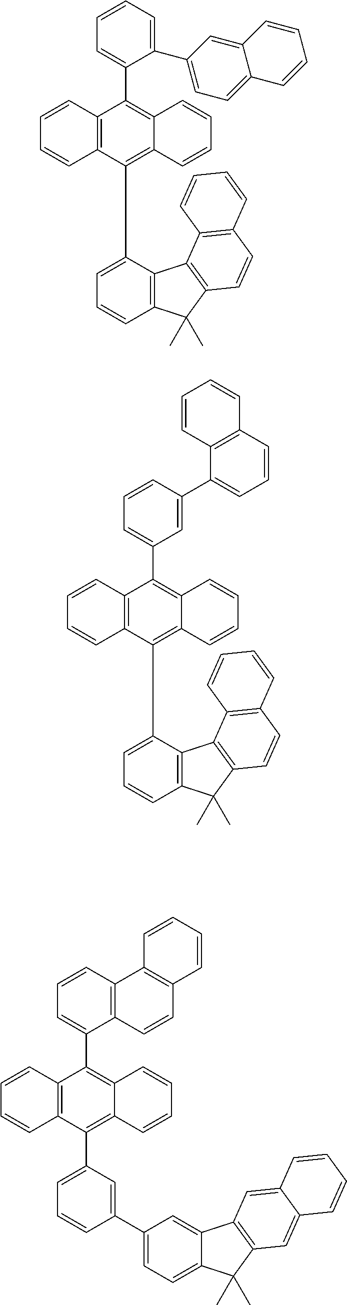

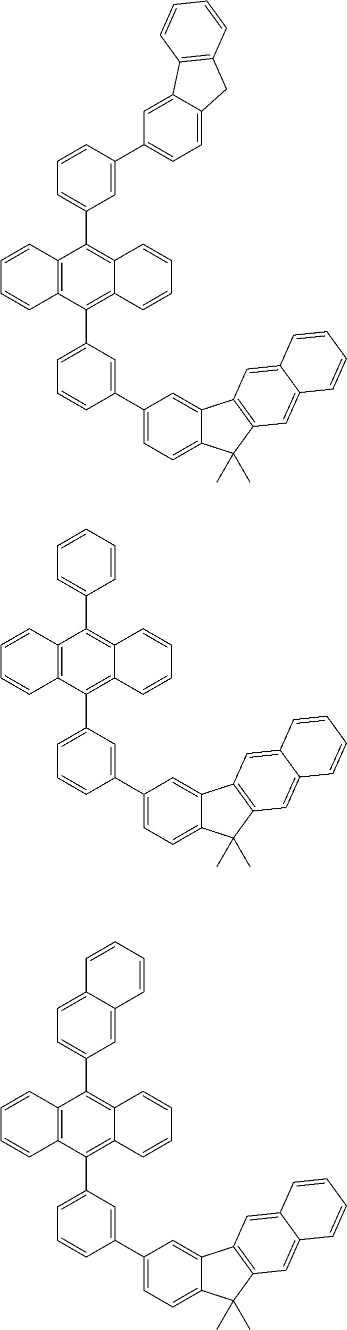

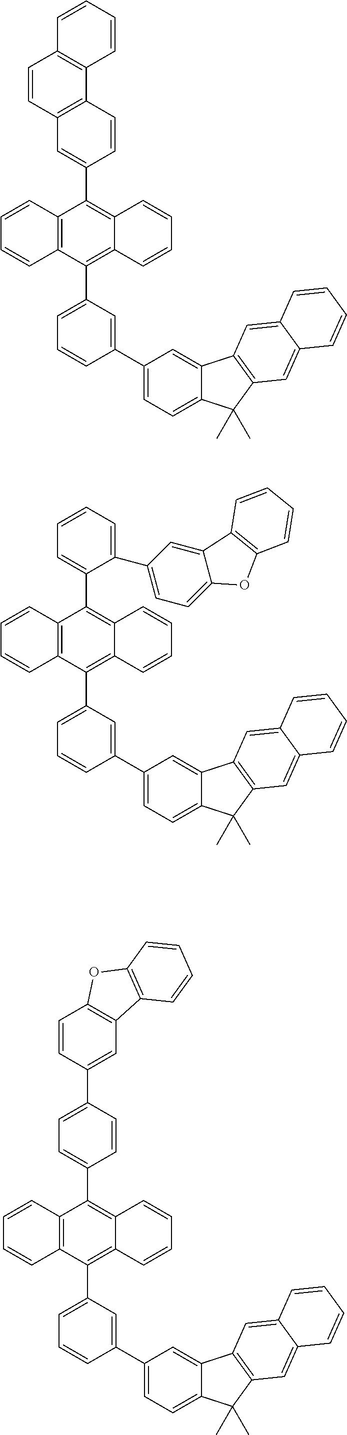

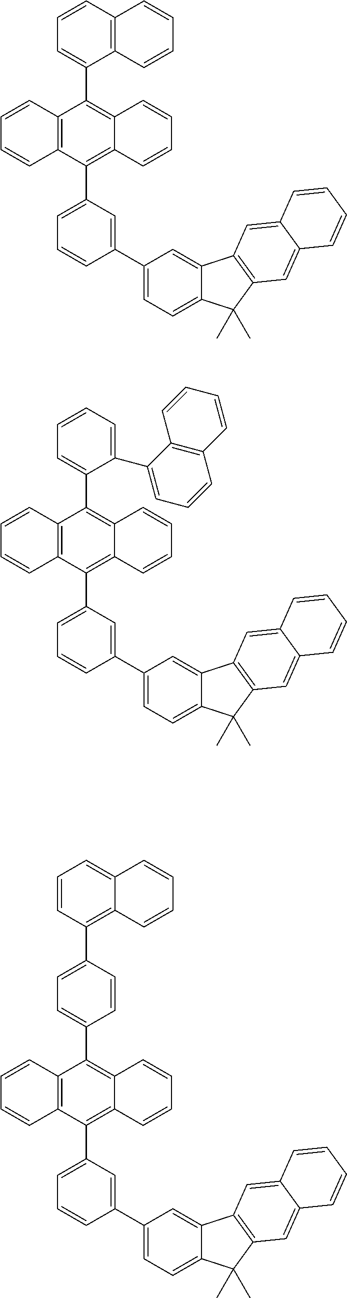

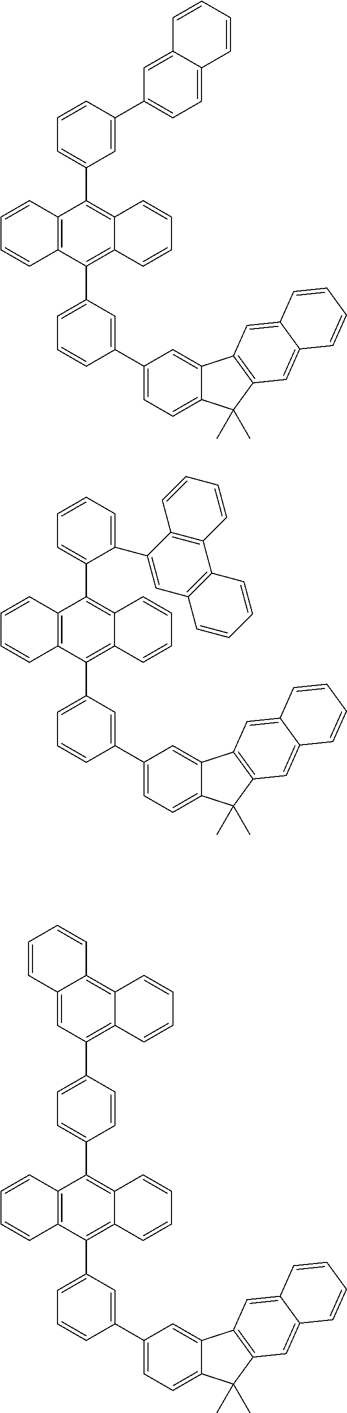

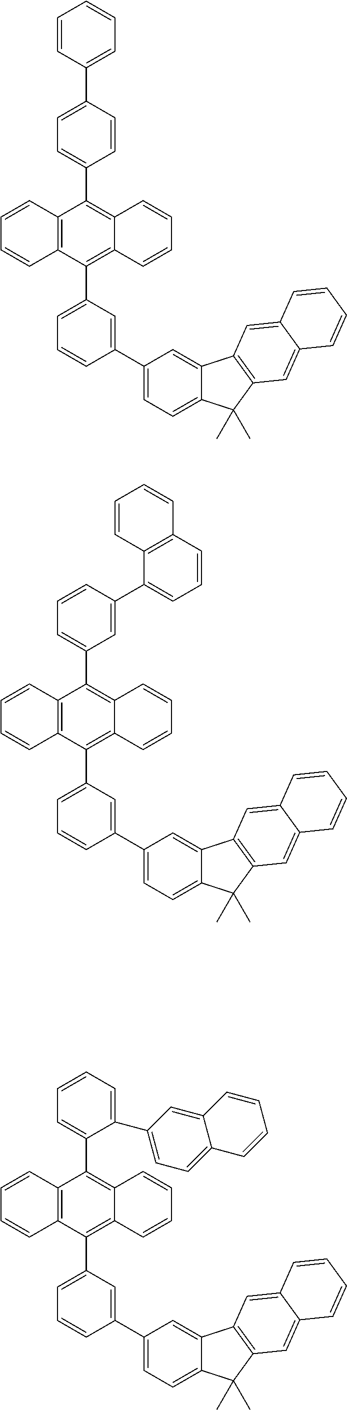

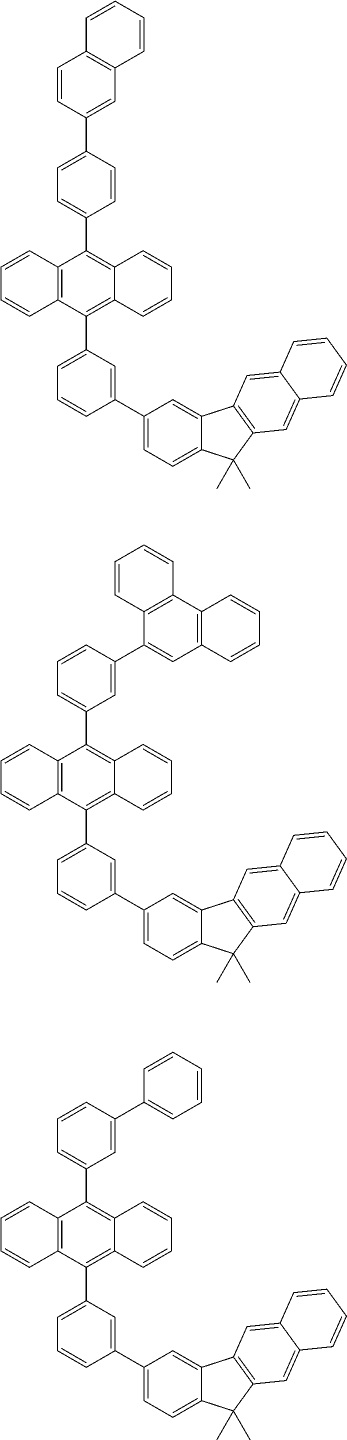

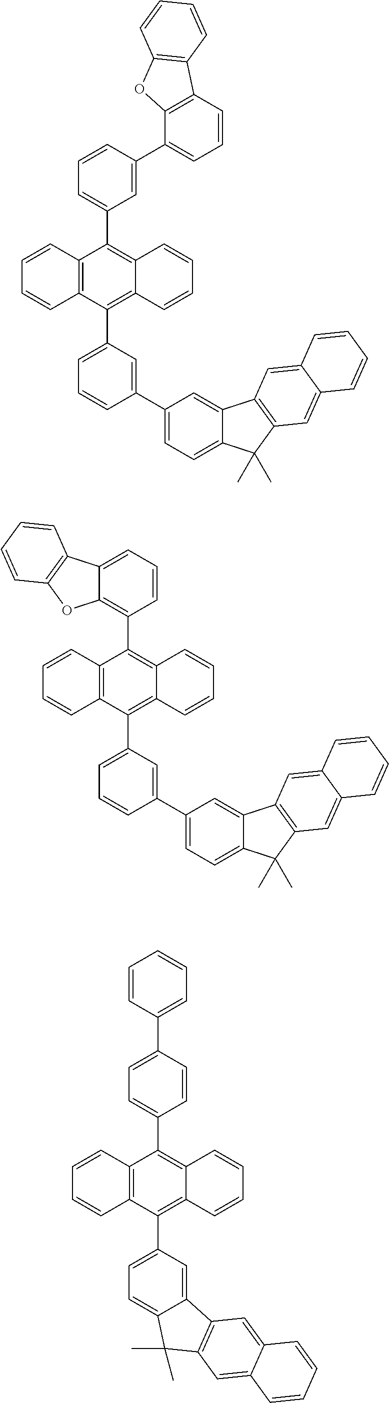

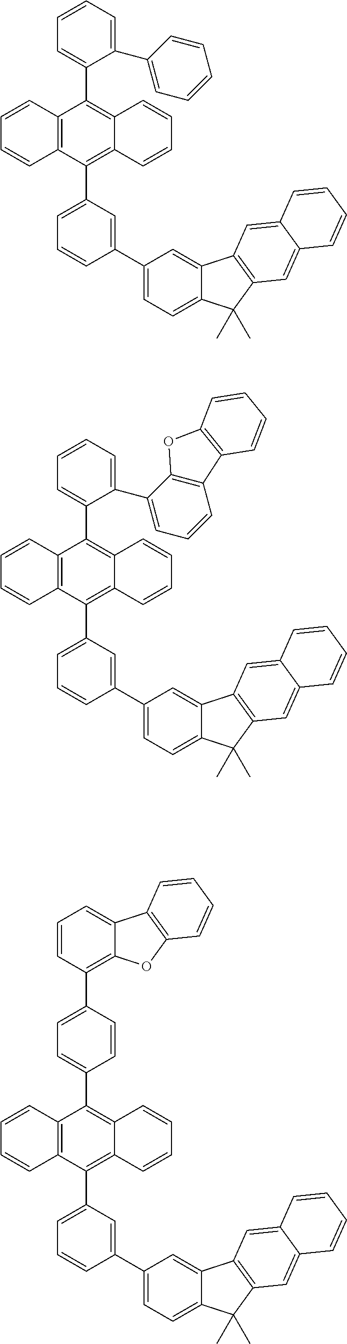

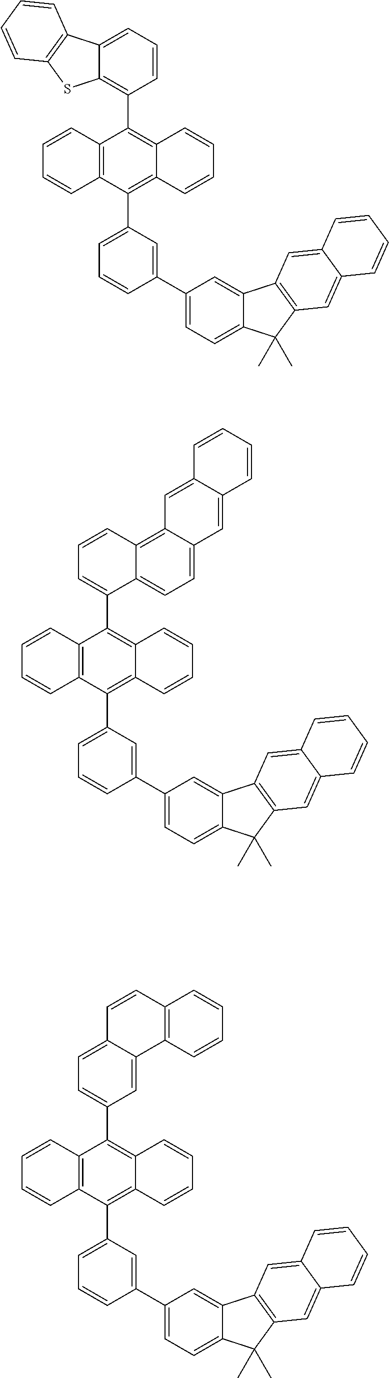

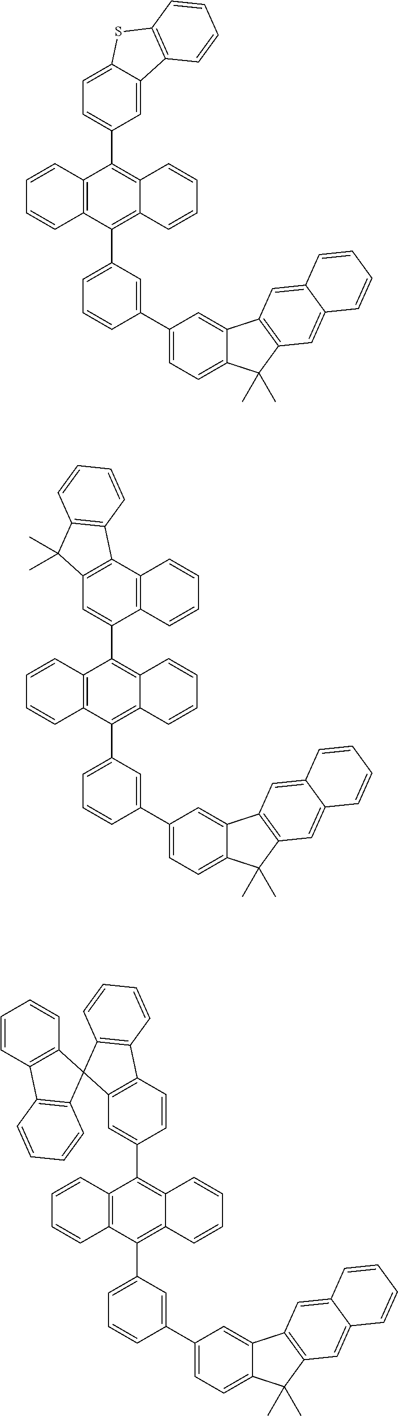

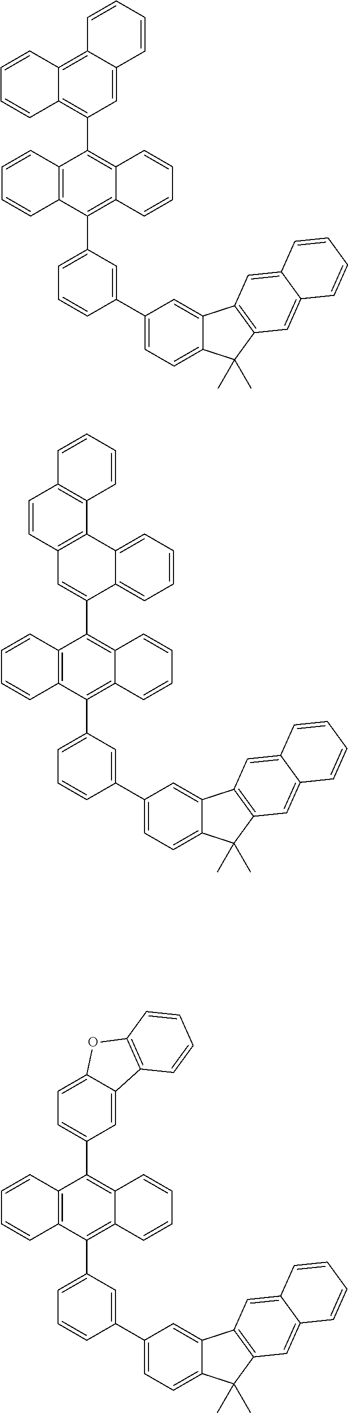

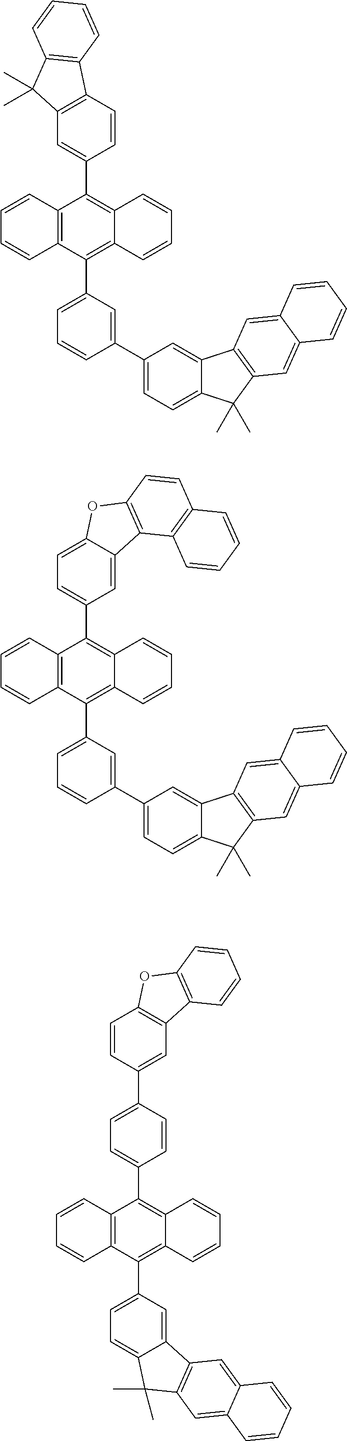

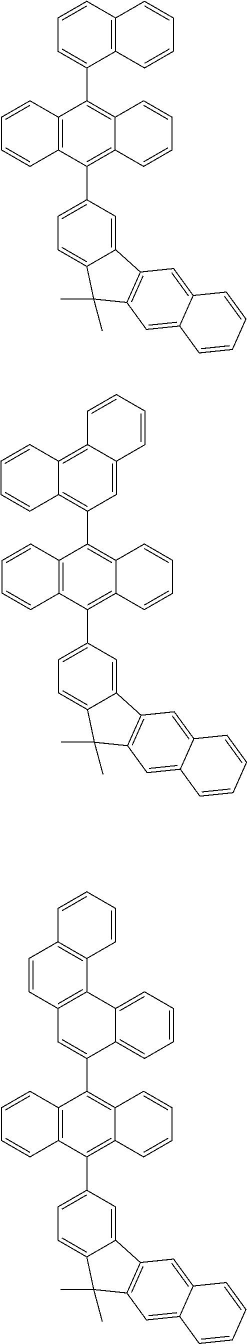

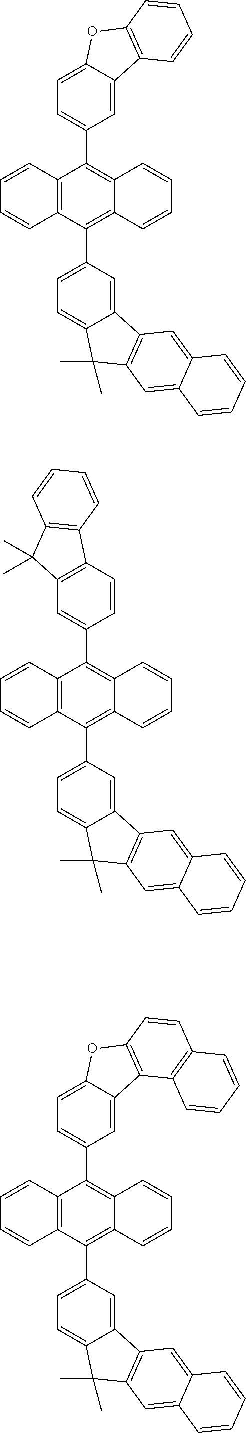

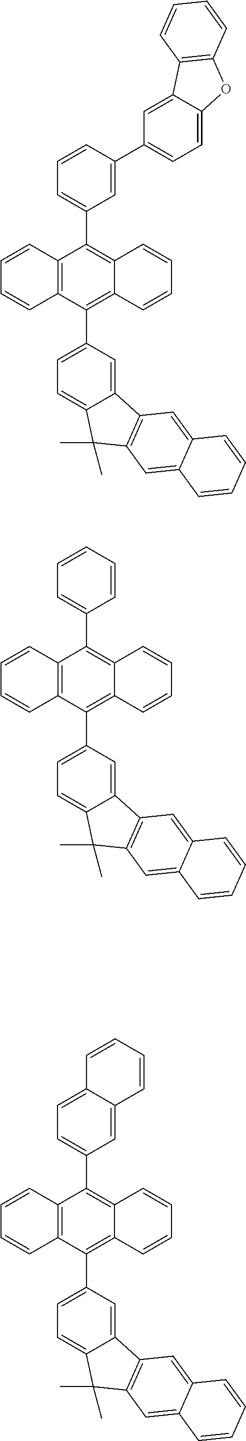

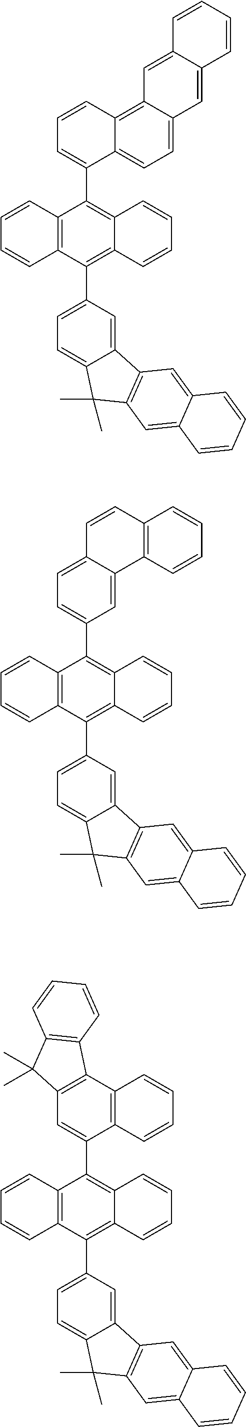

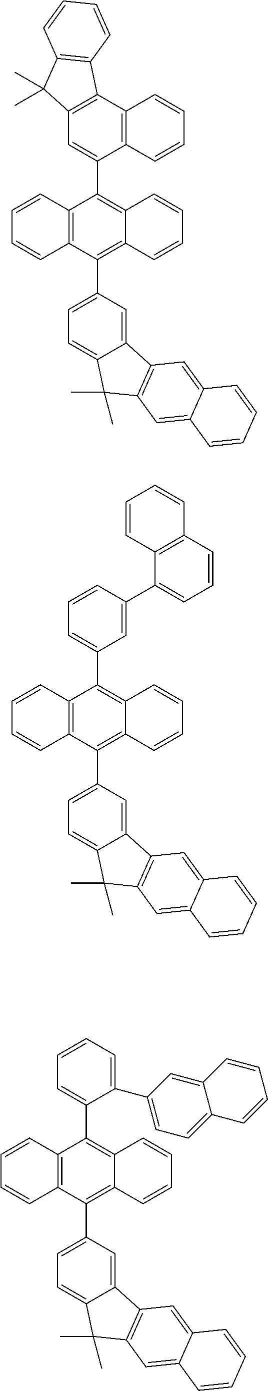

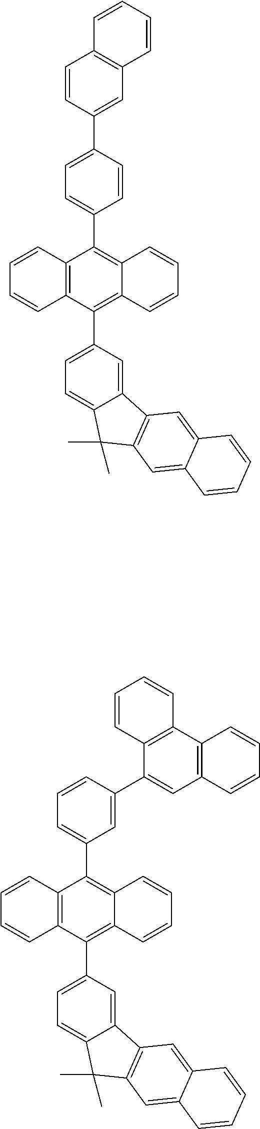

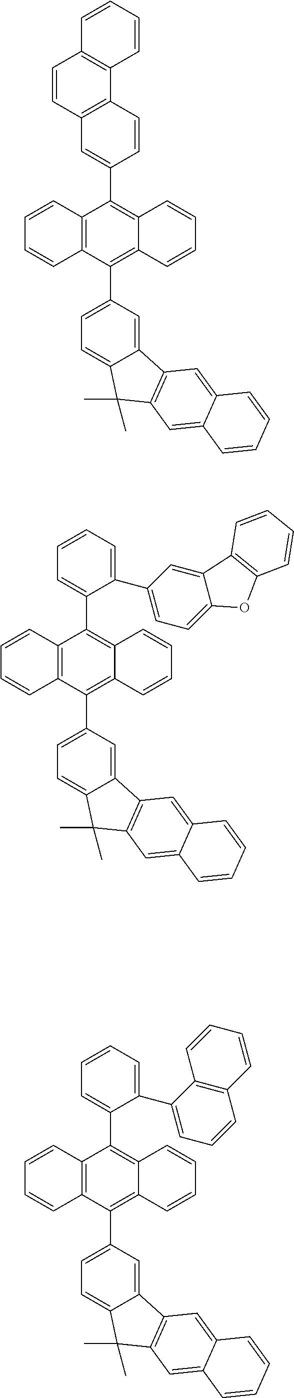

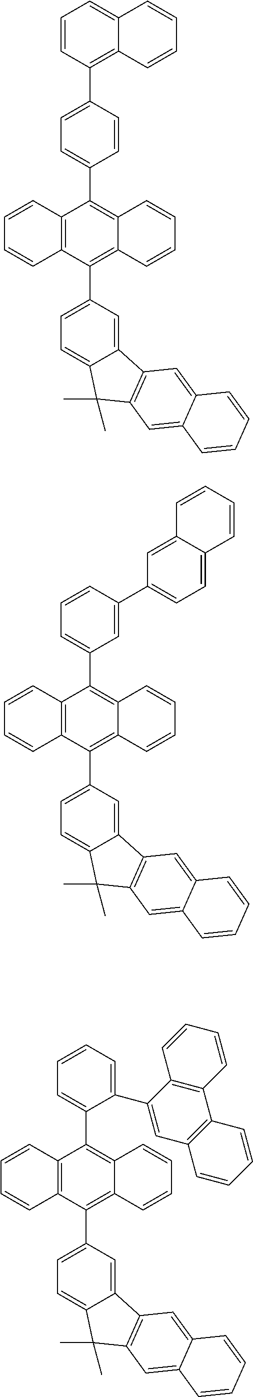

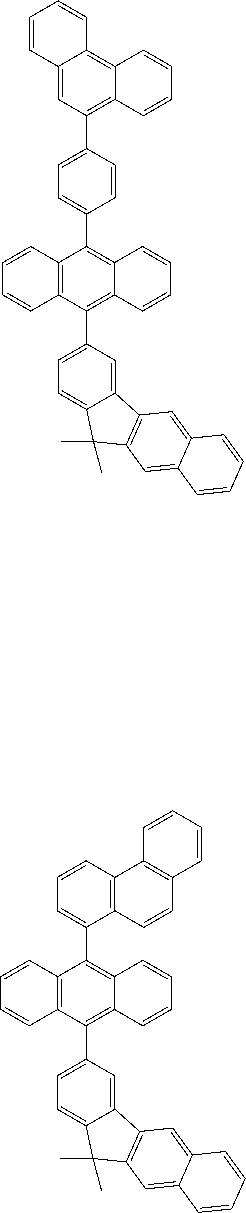

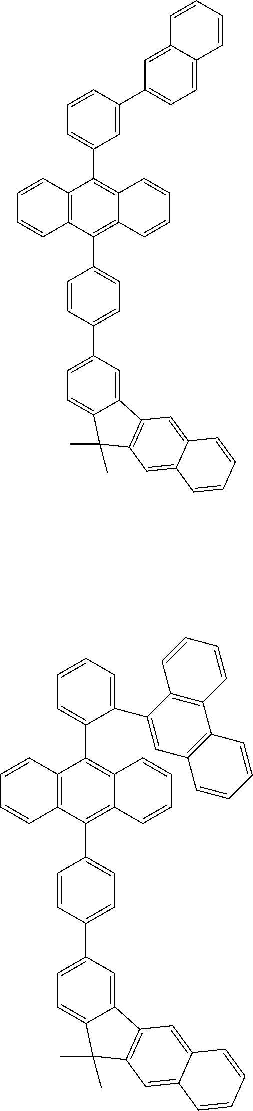

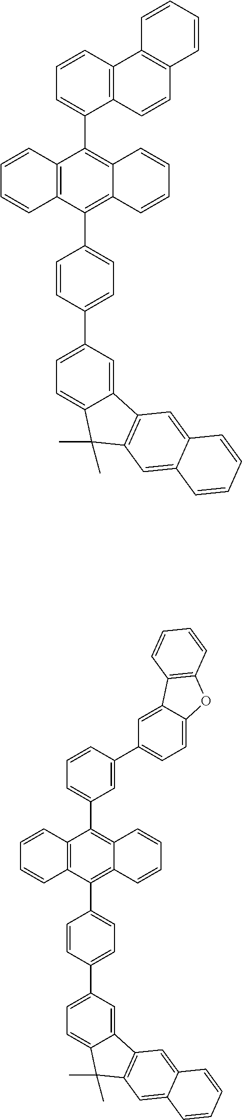

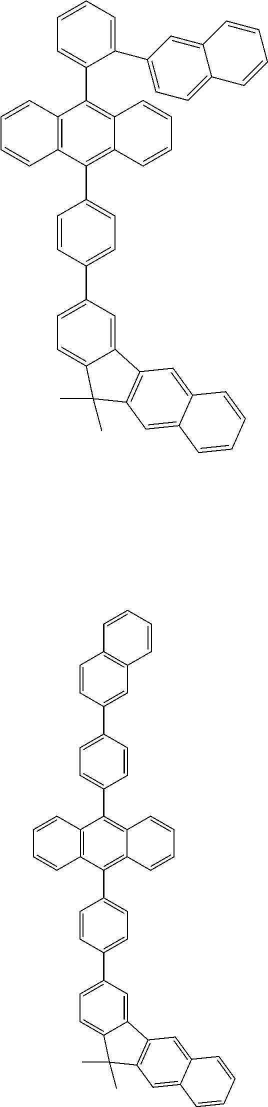

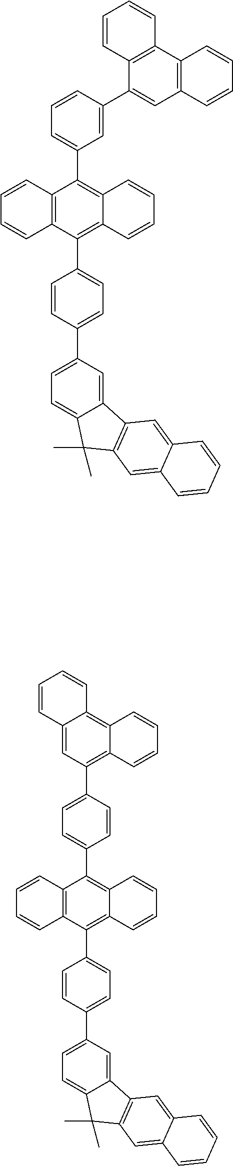

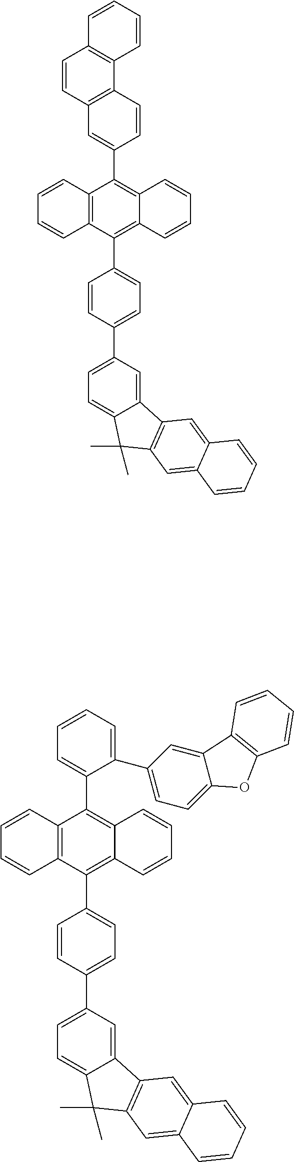

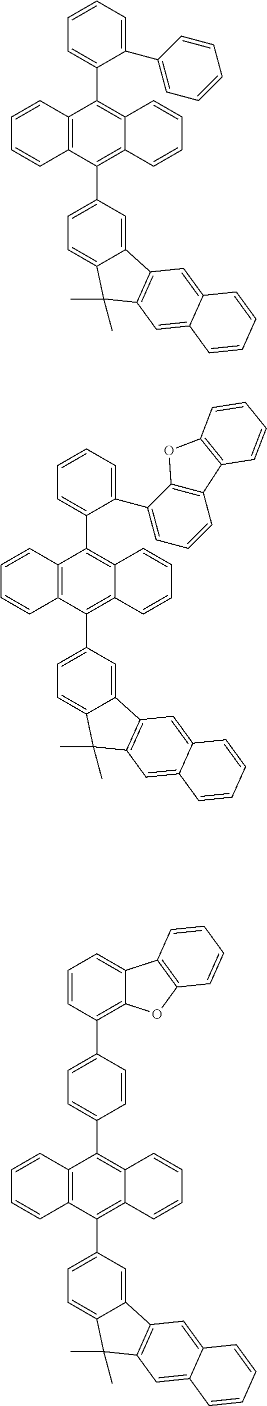

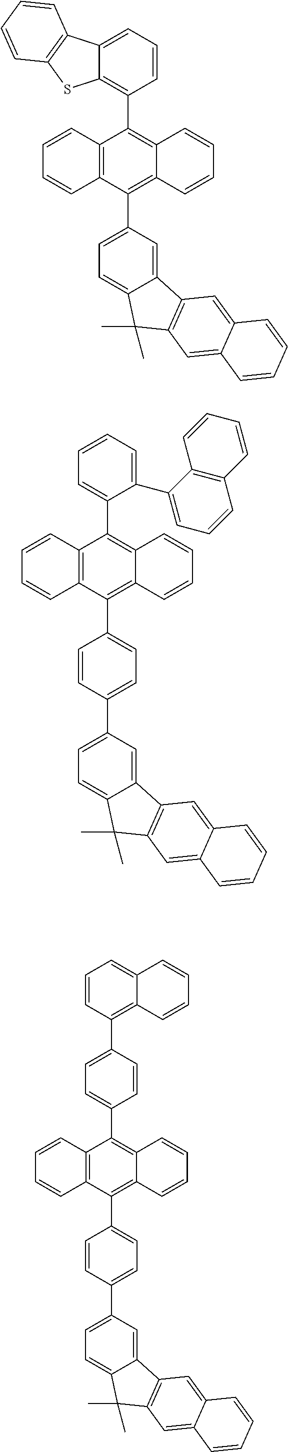

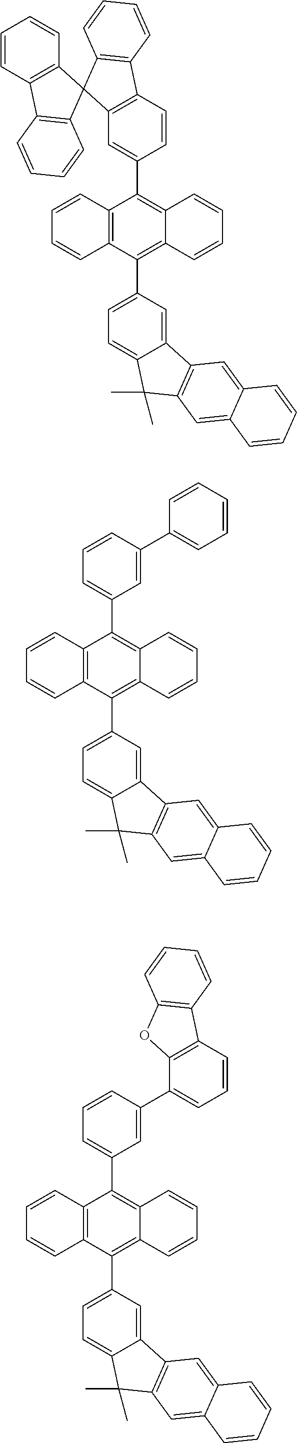

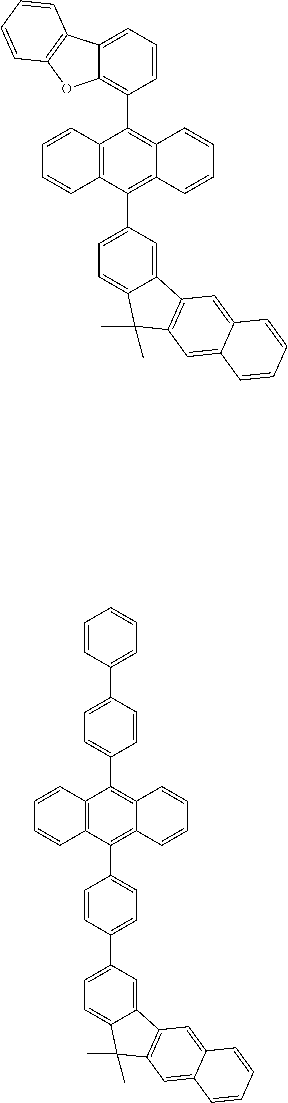

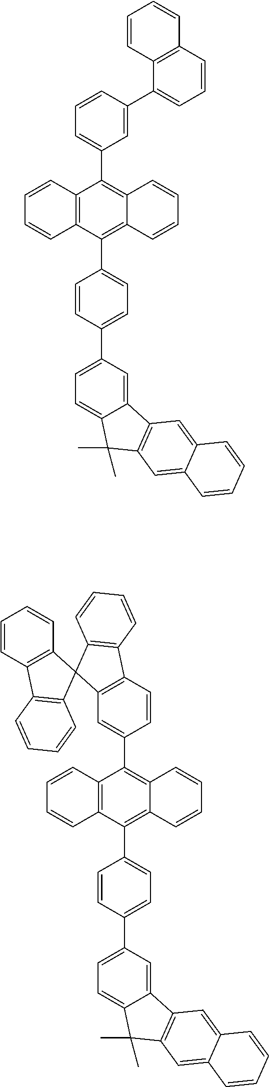

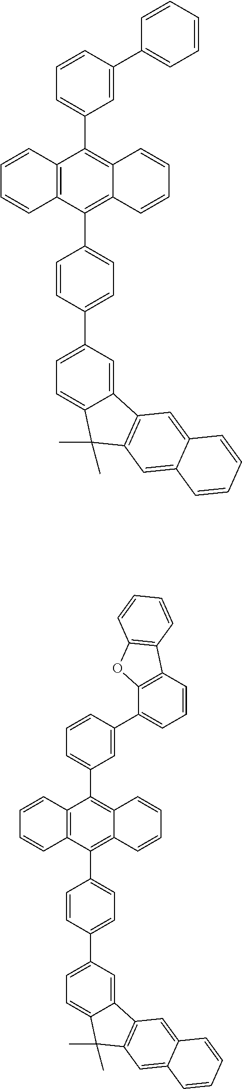

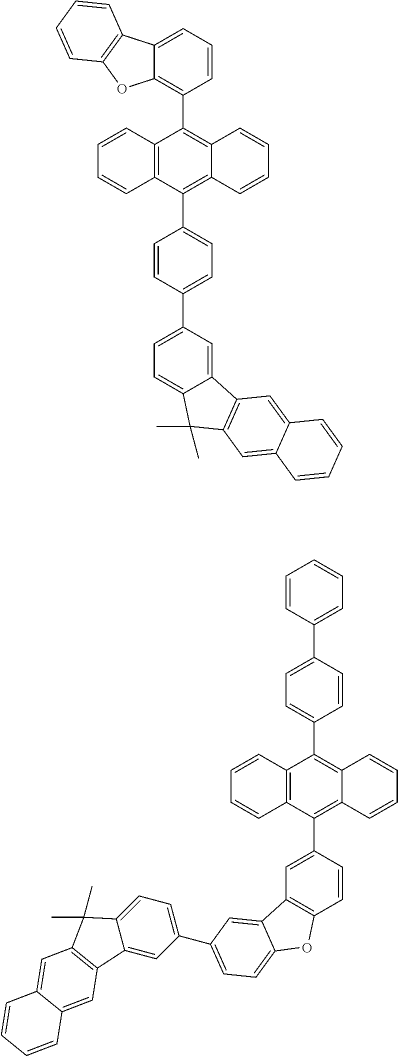 [SMILES: CC1(C)c2ccc(-c3ccc(-c4c5ccccc5c(-c5cccc6c5oc5ccccc56)c5ccccc45)cc3)cc2-c2cc3ccccc3cc21 ; CC1(C)c2ccc(-c3ccc4oc5ccc(-c6c7ccccc7c(-c7ccc(-c8ccccc8)cc7)c7ccccc67)cc5c4c3)cc2-c2cc3ccccc3cc21]

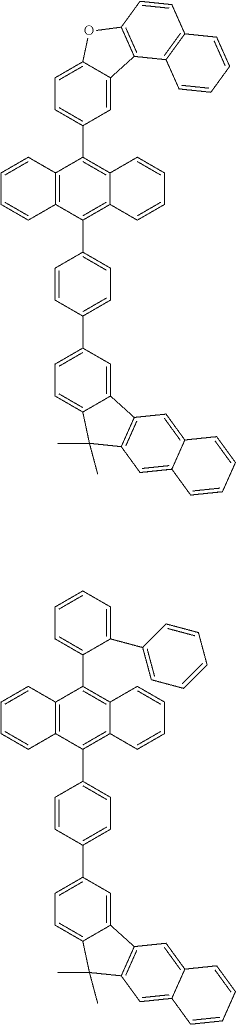

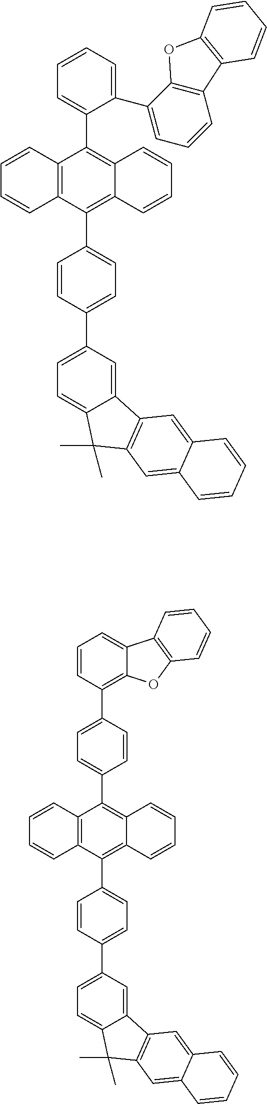 [SMILES: CC1(C)c2ccc(-c3ccc(-c4c5ccccc5c(-c5ccccc5-c5cccc6c5oc5ccccc56)c5ccccc45)cc3)cc2-c2cc3ccccc3cc21 ; CC1(C)c2ccc(-c3ccc(-c4c5ccccc5c(-c5ccc(-c6cccc7c6oc6ccccc67)cc5)c5ccccc45)cc3)cc2-c2cc3ccccc3cc21]

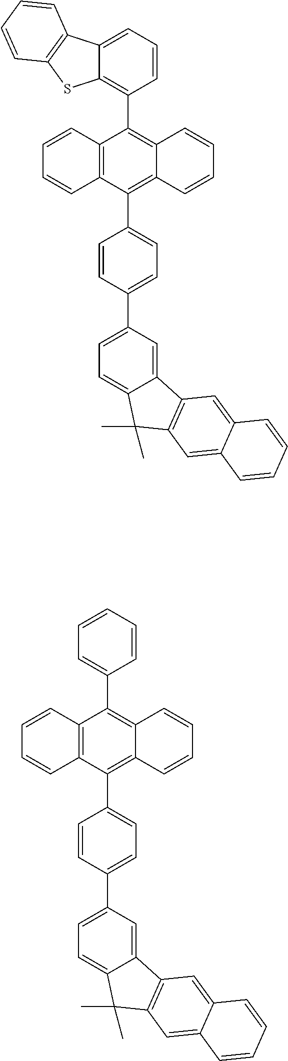

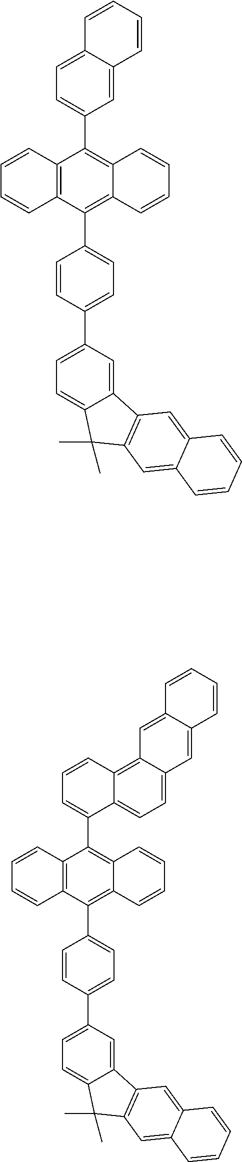

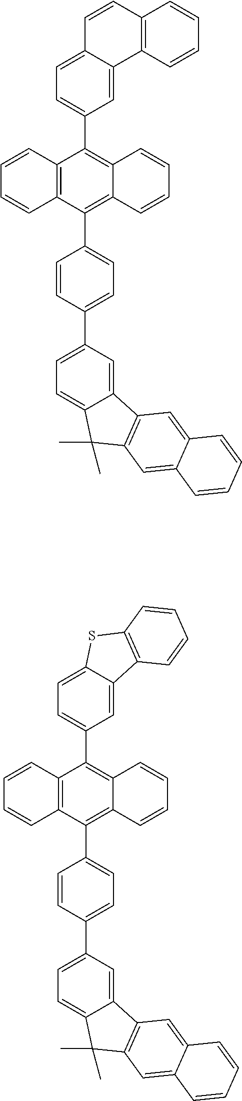

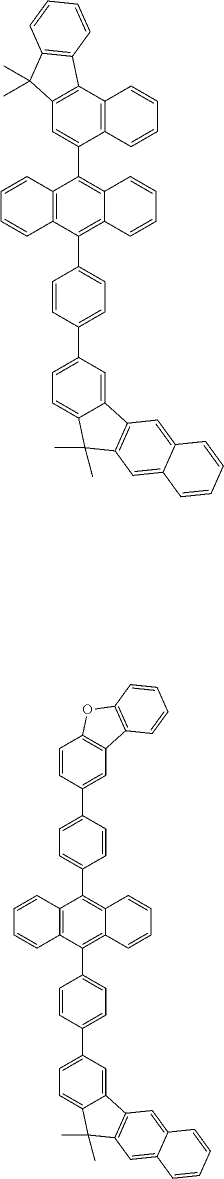

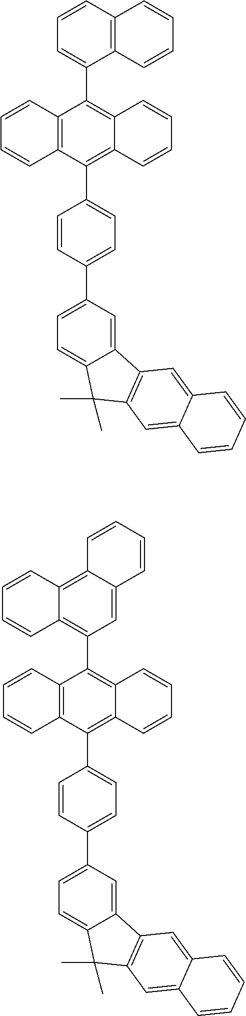

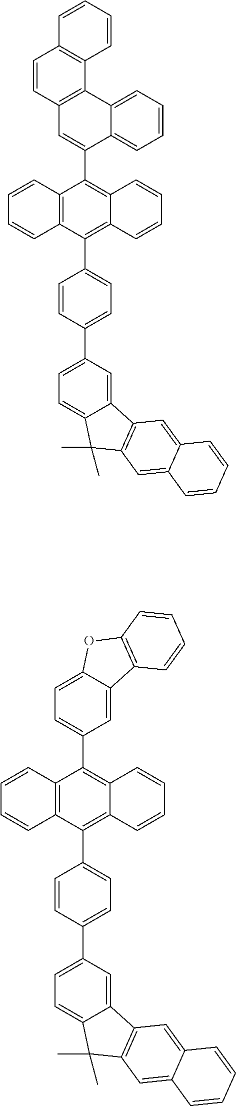

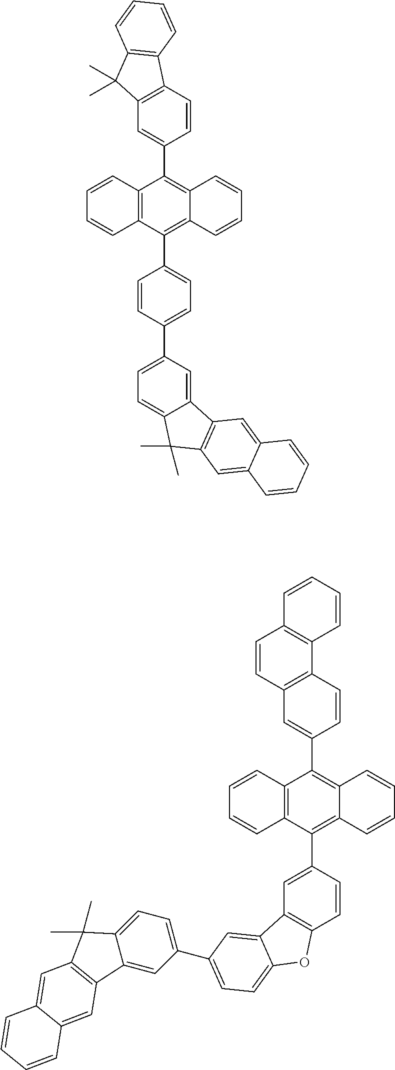

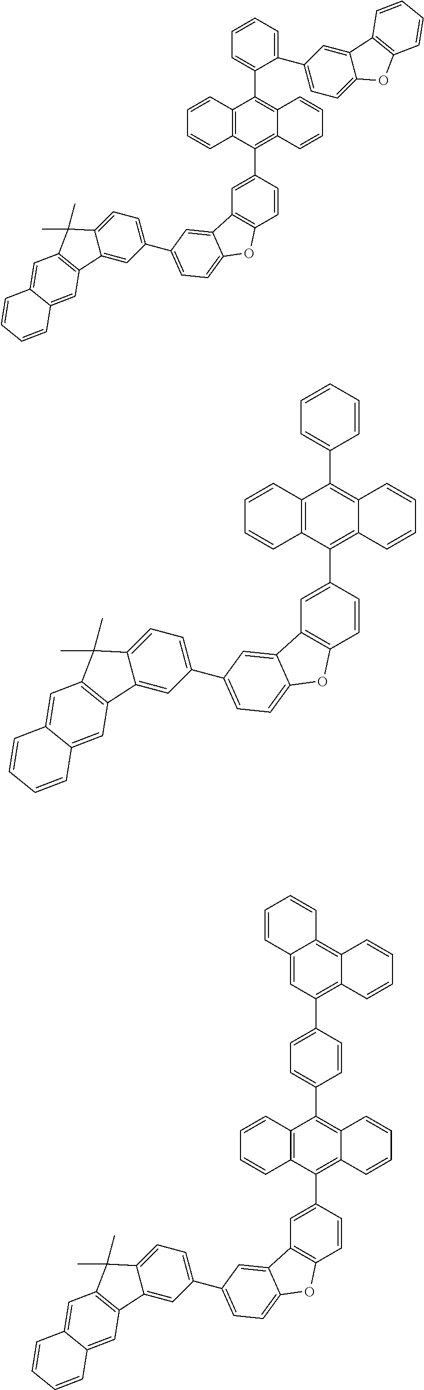 [SMILES: CC1(C)c2ccc(-c3ccc4oc5ccc(-c6c7ccccc7c(-c7ccccc7-c7ccc8oc9ccccc9c8c7)c7ccccc67)cc5c4c3)cc2-c2cc3ccccc3cc21 ; CC1(C)c2ccc(-c3ccc4oc5ccc(-c6c7ccccc7c(-c7ccccc7)c7ccccc67)cc5c4c3)cc2-c2cc3ccccc3cc21 ; CC1(C)c2ccc(-c3ccc4oc5ccc(-c6c7ccccc7c(-c7ccc(-c8cc9ccccc9c9ccccc89)cc7)c7ccccc67)cc5c4c3)cc2-c2cc3ccccc3cc21]

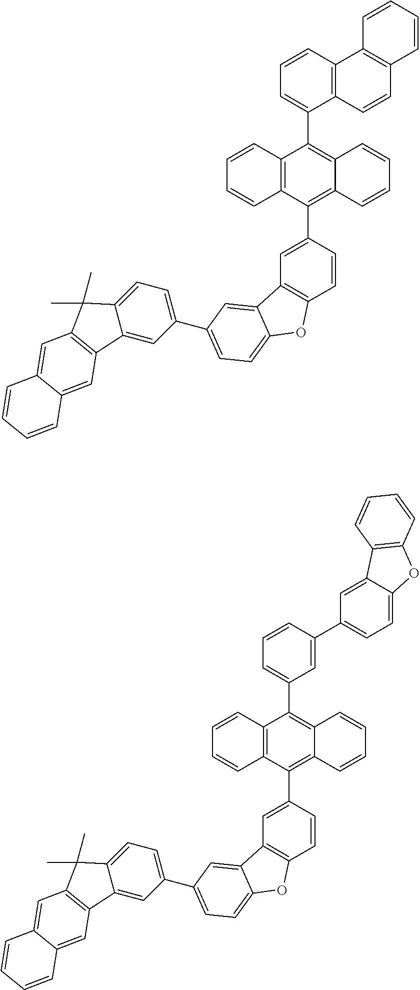 [SMILES: CC1(C)c2ccc(-c3ccc4oc5ccc(-c6c7ccccc7c(-c7cccc8c7ccc7ccccc78)c7ccccc67)cc5c4c3)cc2-c2cc3ccccc3cc21 ; CC1(C)c2ccc(-c3ccc4oc5ccc(-c6c7ccccc7c(-c7cccc(-c8ccc9oc%10ccccc%10c9c8)c7)c7ccccc67)cc5c4c3)cc2-c2cc3ccccc3cc21]

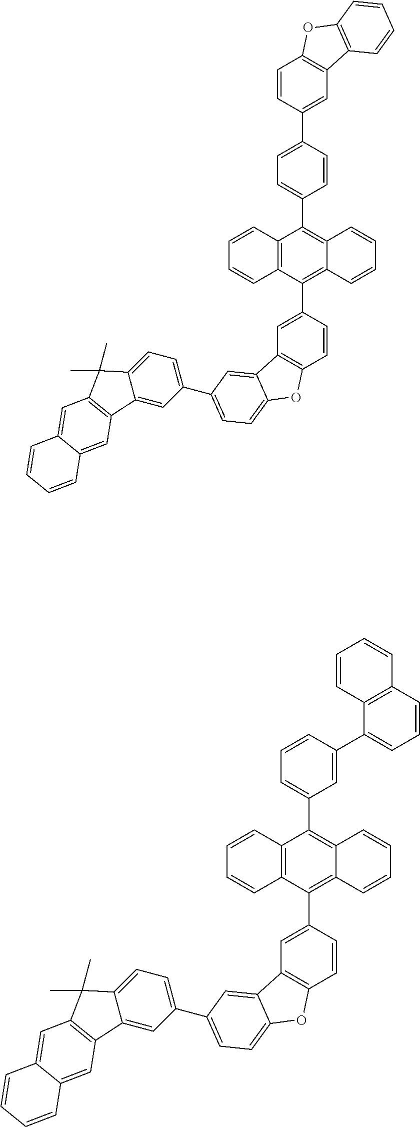 [SMILES: CC1(C)c2ccc(-c3ccc4oc5ccc(-c6c7ccccc7c(-c7ccc(-c8ccc9oc%10ccccc%10c9c8)cc7)c7ccccc67)cc5c4c3)cc2-c2cc3ccccc3cc21 ; CC1(C)c2ccc(-c3ccc4oc5ccc(-c6c7ccccc7c(-c7cccc(-c8cccc9ccccc89)c7)c7ccccc67)cc5c4c3)cc2-c2cc3ccccc3cc21]

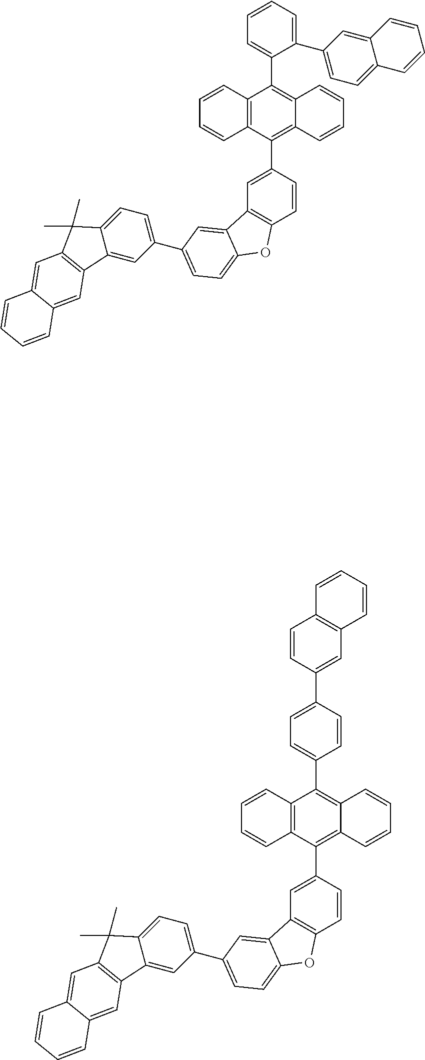 [SMILES: CC1(C)c2ccc(-c3ccc4oc5ccc(-c6c7ccccc7c(-c7ccccc7-c7ccc8ccccc8c7)c7ccccc67)cc5c4c3)cc2-c2cc3ccccc3cc21 ; CC1(C)c2ccc(-c3ccc4oc5ccc(-c6c7ccccc7c(-c7ccc(-c8ccc9ccccc9c8)cc7)c7ccccc67)cc5c4c3)cc2-c2cc3ccccc3cc21]

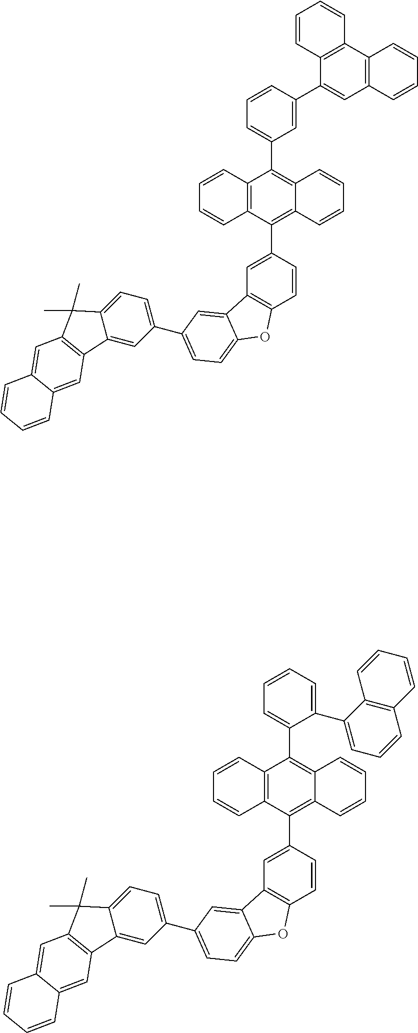 [SMILES: CC1(C)c2ccc(-c3ccc4oc5ccc(-c6c7ccccc7c(-c7cccc(-c8cc9ccccc9c9ccccc89)c7)c7ccccc67)cc5c4c3)cc2-c2cc3ccccc3cc21 ; CC1(C)c2ccc(-c3ccc4oc5ccc(-c6c7ccccc7c(-c7ccccc7-c7cccc8ccccc78)c7ccccc67)cc5c4c3)cc2-c2cc3ccccc3cc21]

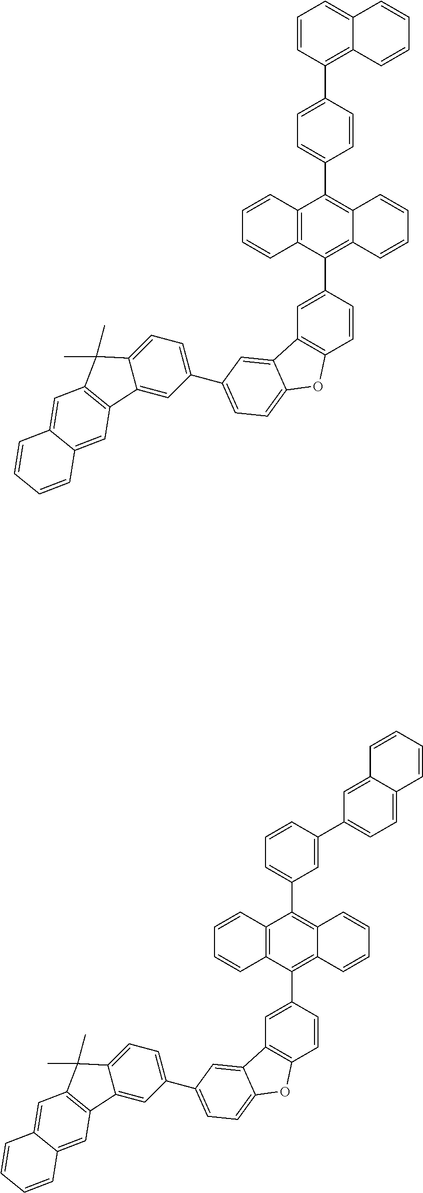 [SMILES: CC1(C)c2ccc(-c3ccc4oc5ccc(-c6c7ccccc7c(-c7ccc(-c8cccc9ccccc89)cc7)c7ccccc67)cc5c4c3)cc2-c2cc3ccccc3cc21 ; CC1(C)c2ccc(-c3ccc4oc5ccc(-c6c7ccccc7c(-c7cccc(-c8ccc9ccccc9c8)c7)c7ccccc67)cc5c4c3)cc2-c2cc3ccccc3cc21]

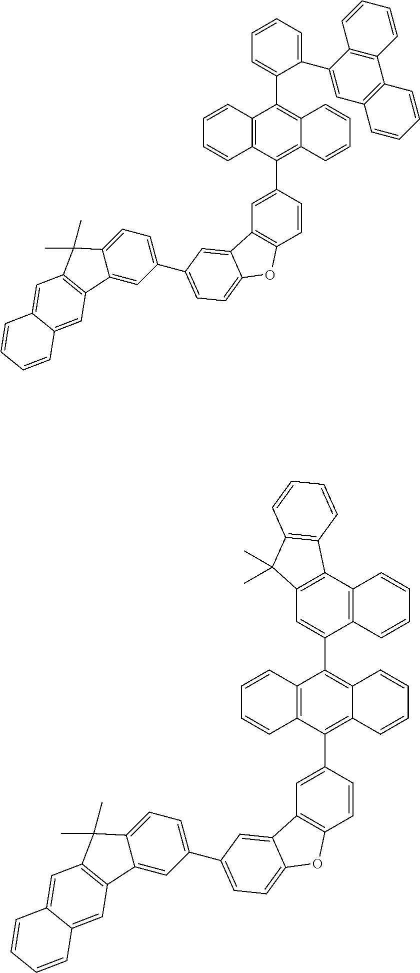 [SMILES: CC1(C)c2ccc(-c3ccc4oc5ccc(-c6c7ccccc7c(-c7ccccc7-c7cc8ccccc8c8ccccc78)c7ccccc67)cc5c4c3)cc2-c2cc3ccccc3cc21 ; CC1(C)c2ccc(-c3ccc4oc5ccc(-c6c7ccccc7c(-c7cc8c(c9ccccc79)-c7ccccc7C8(C)C)c7ccccc67)cc5c4c3)cc2-c2cc3ccccc3cc21]

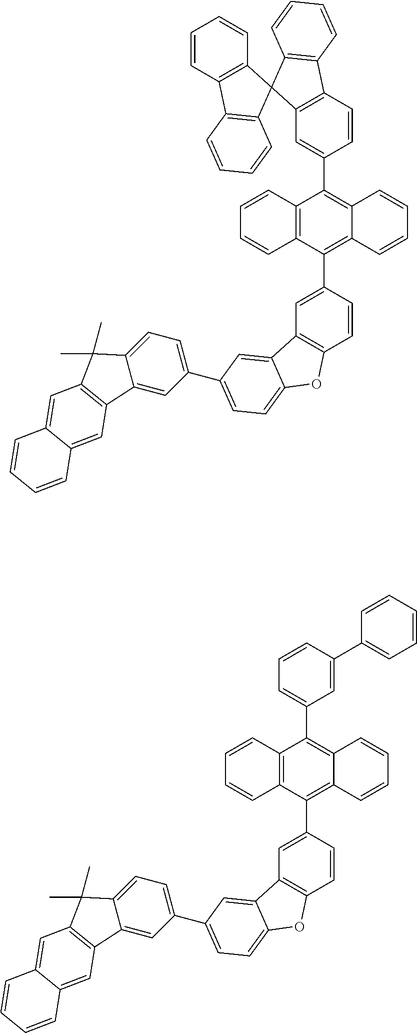 [SMILES: CC1(C)c2ccc(-c3ccc4oc5ccc(-c6c7ccccc7c(-c7ccc8c(c7)C7(c9ccccc9-c9ccccc97)c7ccccc7-8)c7ccccc67)cc5c4c3)cc2-c2cc3ccccc3cc21 ; CC1(C)c2ccc(-c3ccc4oc5ccc(-c6c7ccccc7c(-c7cccc(-c8ccccc8)c7)c7ccccc67)cc5c4c3)cc2-c2cc3ccccc3cc21]

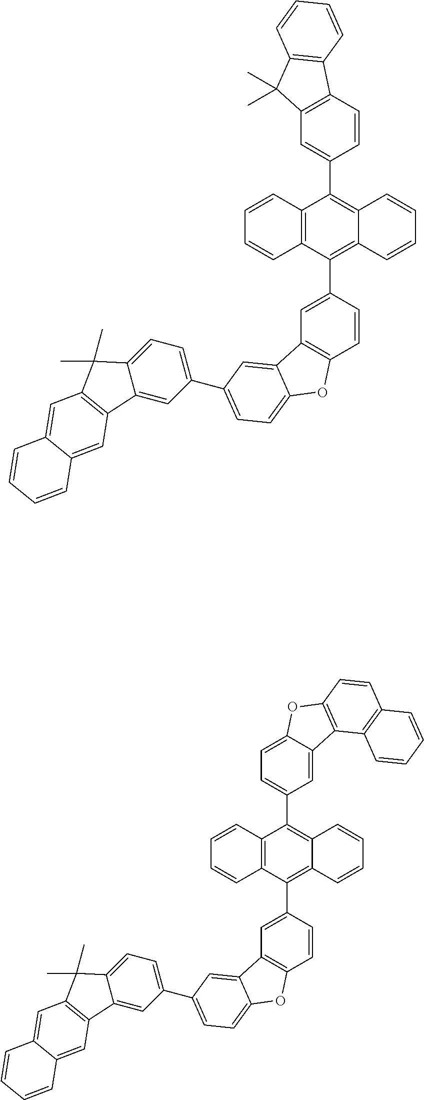 [SMILES: CC1(C)c2ccccc2-c2ccc(-c3c4ccccc4c(-c4ccc5oc6ccc(-c7ccc8c(c7)-c7cc9ccccc9cc7C8(C)C)cc6c5c4)c4ccccc34)cc21 ; CC1(C)c2ccc(-c3ccc4oc5ccc(-c6c7ccccc7c(-c7ccc8oc9ccc%10ccccc%10c9c8c7)c7ccccc67)cc5c4c3)cc2-c2cc3ccccc3cc21]

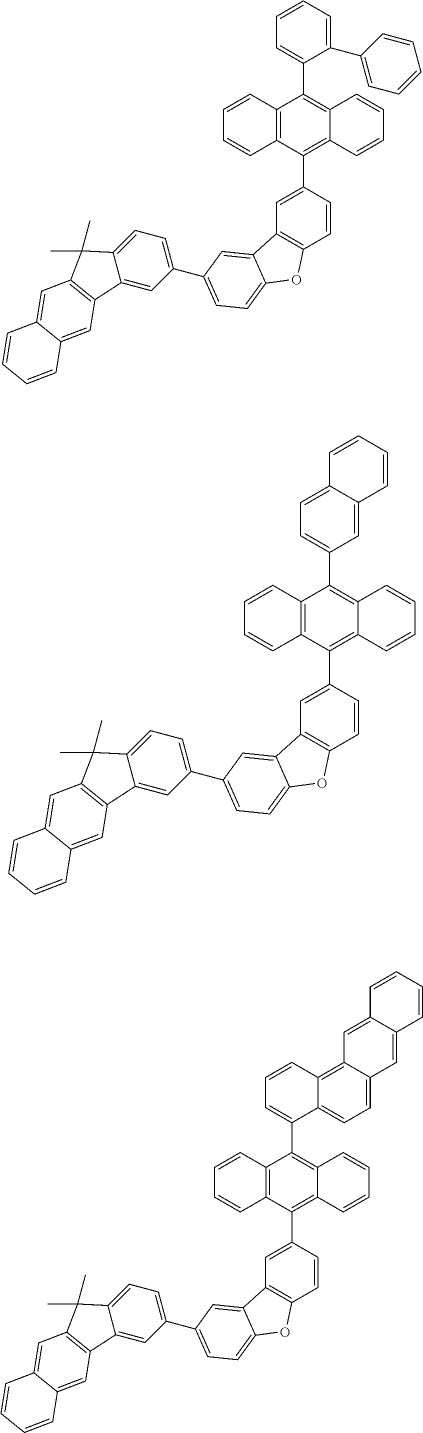 [SMILES: CC1(C)c2ccc(-c3ccc4oc5ccc(-c6c7ccccc7c(-c7ccccc7-c7ccccc7)c7ccccc67)cc5c4c3)cc2-c2cc3ccccc3cc21 ; CC1(C)c2ccc(-c3ccc4oc5ccc(-c6c7ccccc7c(-c7ccc8ccccc8c7)c7ccccc67)cc5c4c3)cc2-c2cc3ccccc3cc21 ; CC1(C)c2ccc(-c3ccc4oc5ccc(-c6c7ccccc7c(-c7cccc8c7ccc7cc9ccccc9cc78)c7ccccc67)cc5c4c3)cc2-c2cc3ccccc3cc21]

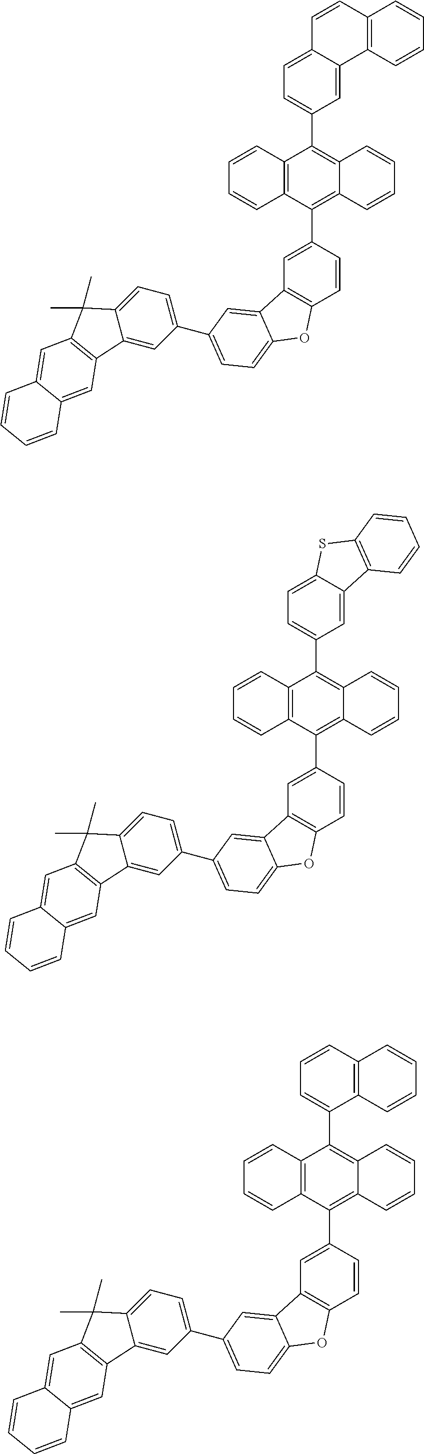 [SMILES: CC1(C)c2ccc(-c3ccc4oc5ccc(-c6c7ccccc7c(-c7ccc8ccc9ccccc9c8c7)c7ccccc67)cc5c4c3)cc2-c2cc3ccccc3cc21 ; CC1(C)c2ccc(-c3ccc4oc5ccc(-c6c7ccccc7c(-c7ccc8sc9ccccc9c8c7)c7ccccc67)cc5c4c3)cc2-c2cc3ccccc3cc21 ; CC1(C)c2ccc(-c3ccc4oc5ccc(-c6c7ccccc7c(-c7cccc8ccccc78)c7ccccc67)cc5c4c3)cc2-c2cc3ccccc3cc21]

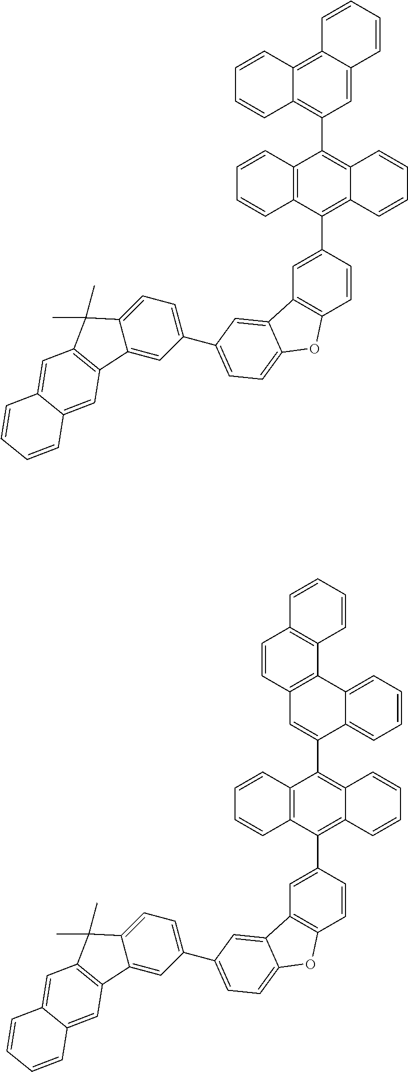 [SMILES: CC1(C)c2ccc(-c3ccc4oc5ccc(-c6c7ccccc7c(-c7cc8ccccc8c8ccccc78)c7ccccc67)cc5c4c3)cc2-c2cc3ccccc3cc21 ; CC1(C)c2ccc(-c3ccc4oc5ccc(-c6c7ccccc7c(-c7cc8ccc9ccccc9c8c8ccccc78)c7ccccc67)cc5c4c3)cc2-c2cc3ccccc3cc21]

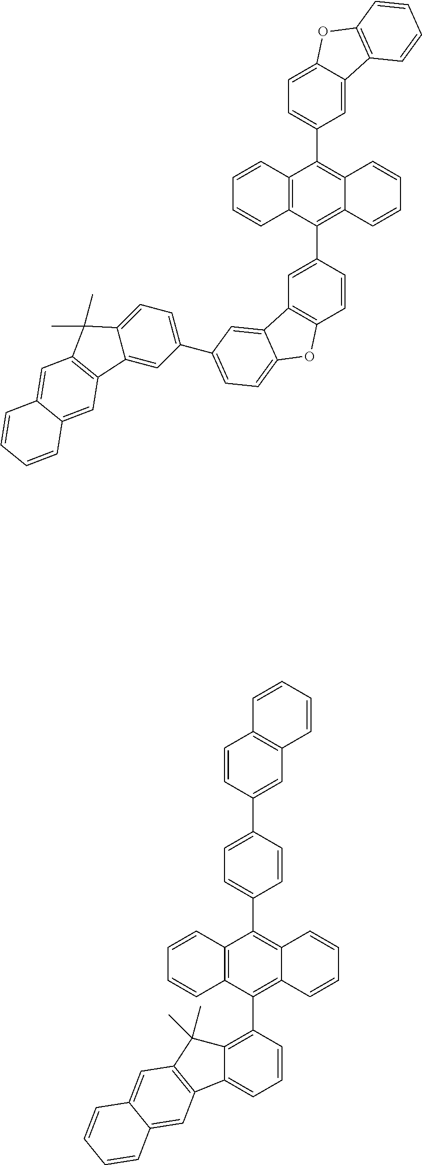

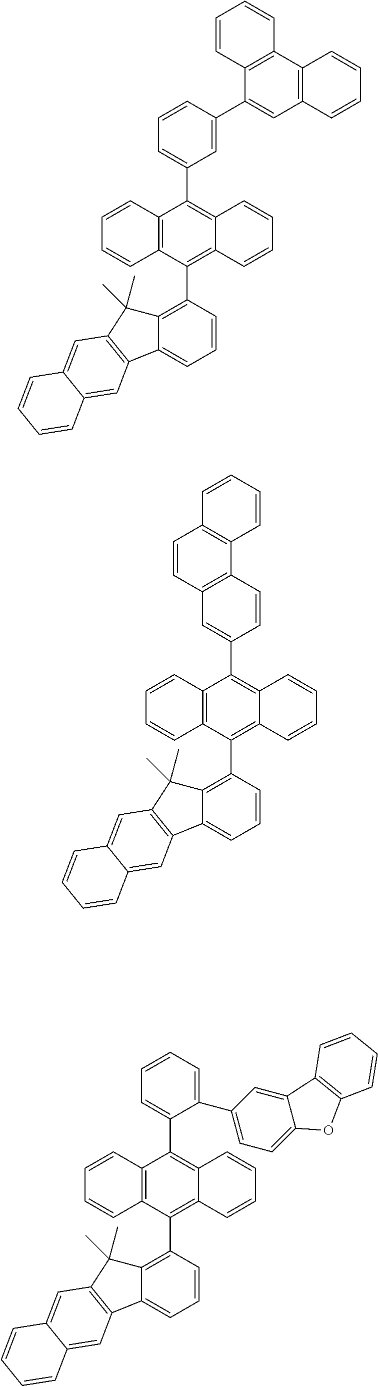

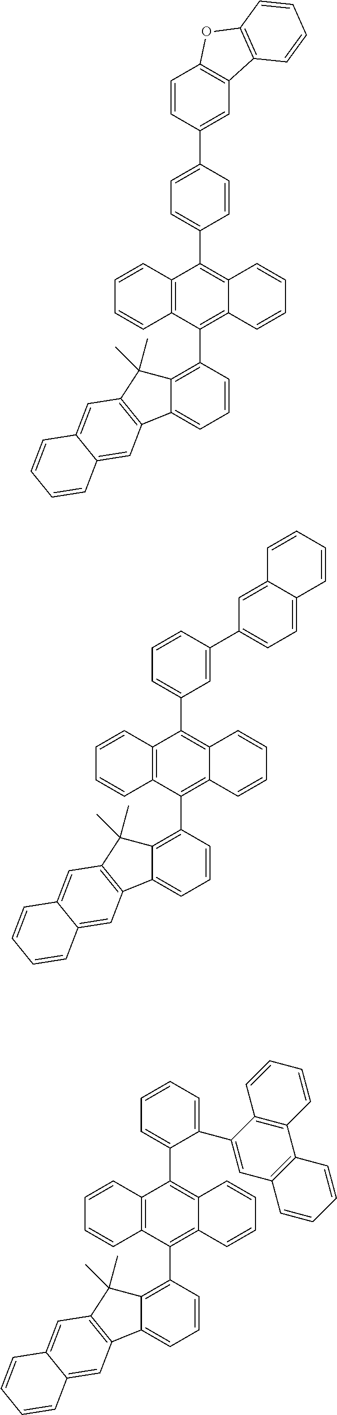

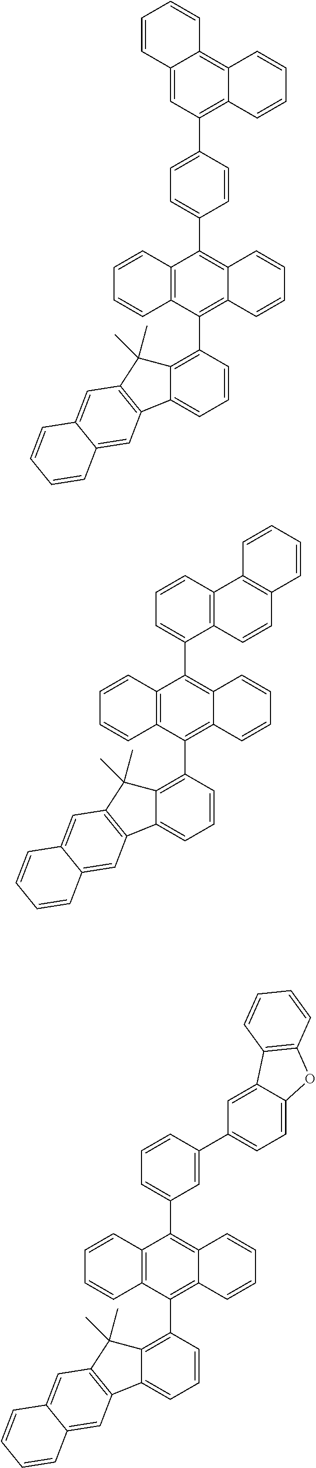

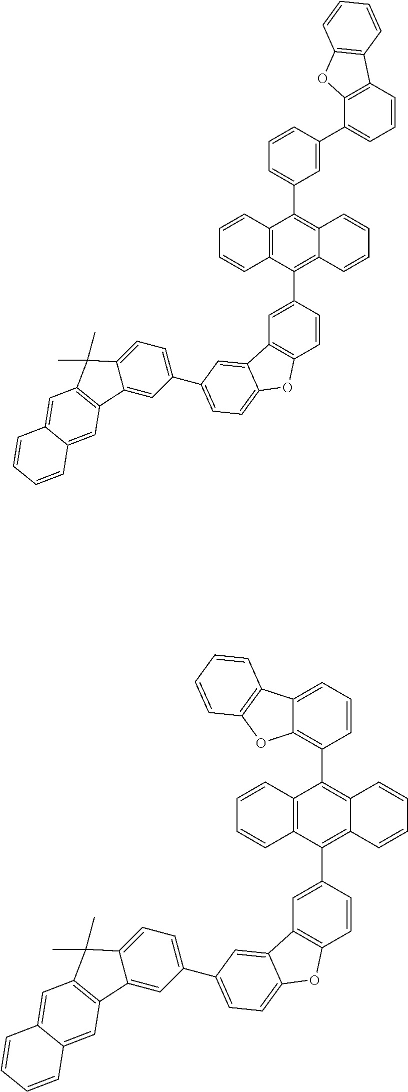 [SMILES: CC1(C)c2ccc(-c3ccc4oc5ccc(-c6c7ccccc7c(-c7cccc(-c8cccc9c8oc8ccccc89)c7)c7ccccc67)cc5c4c3)cc2-c2cc3ccccc3cc21 ; CC1(C)c2ccc(-c3ccc4oc5ccc(-c6c7ccccc7c(-c7cccc8c7oc7ccccc78)c7ccccc67)cc5c4c3)cc2-c2cc3ccccc3cc21]

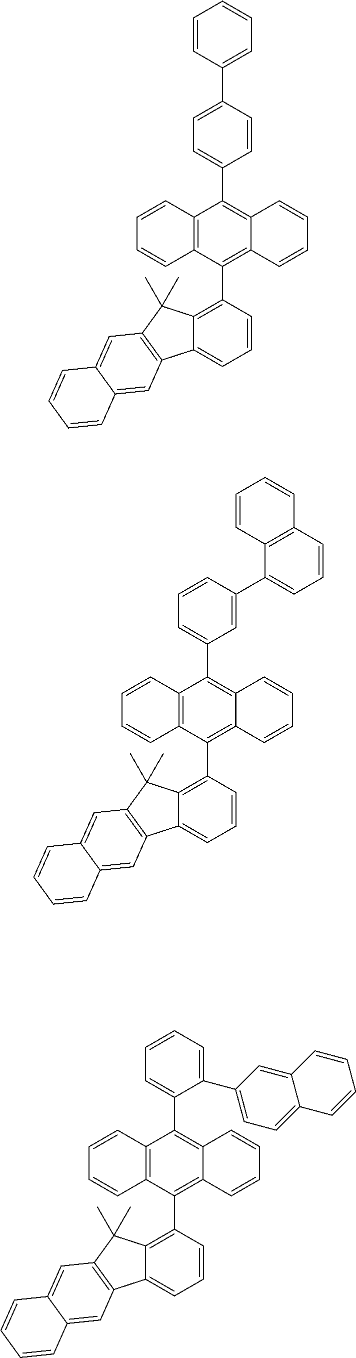

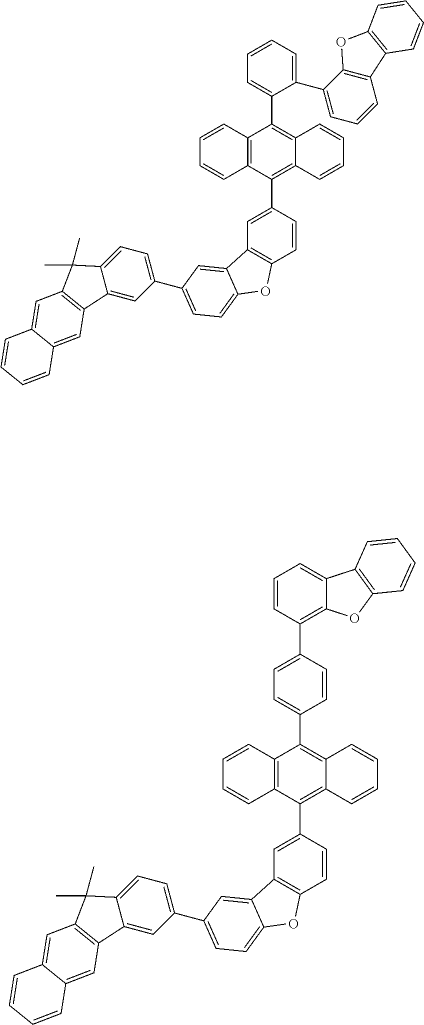 [SMILES: CC1(C)c2ccc(-c3ccc4oc5ccc(-c6c7ccccc7c(-c7ccccc7-c7cccc8c7oc7ccccc78)c7ccccc67)cc5c4c3)cc2-c2cc3ccccc3cc21 ; CC1(C)c2ccc(-c3ccc4oc5ccc(-c6c7ccccc7c(-c7ccc(-c8cccc9c8oc8ccccc89)cc7)c7ccccc67)cc5c4c3)cc2-c2cc3ccccc3cc21]

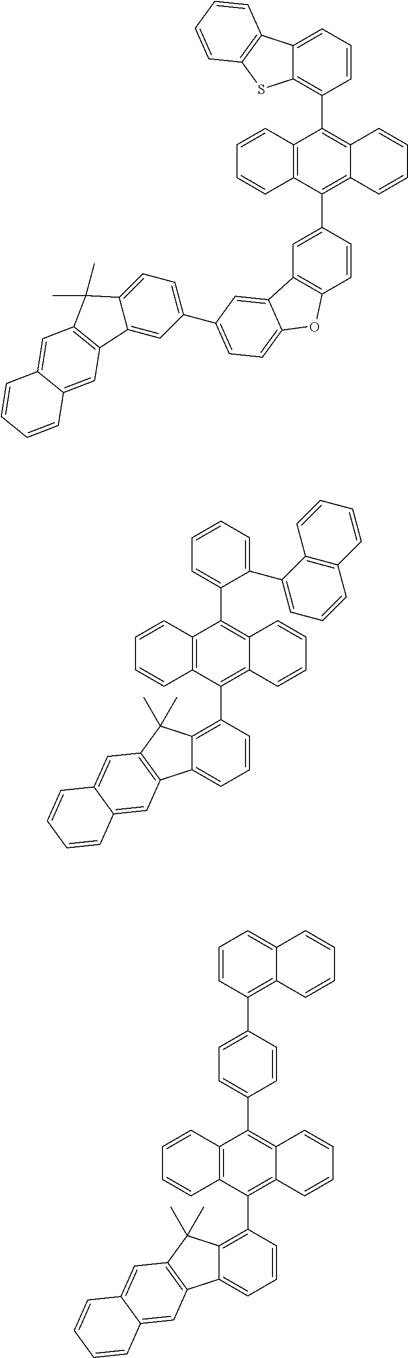

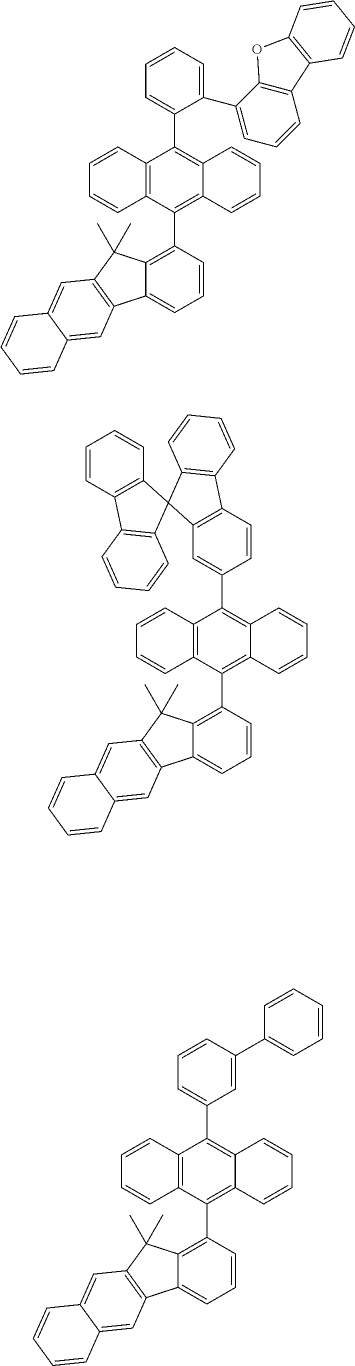

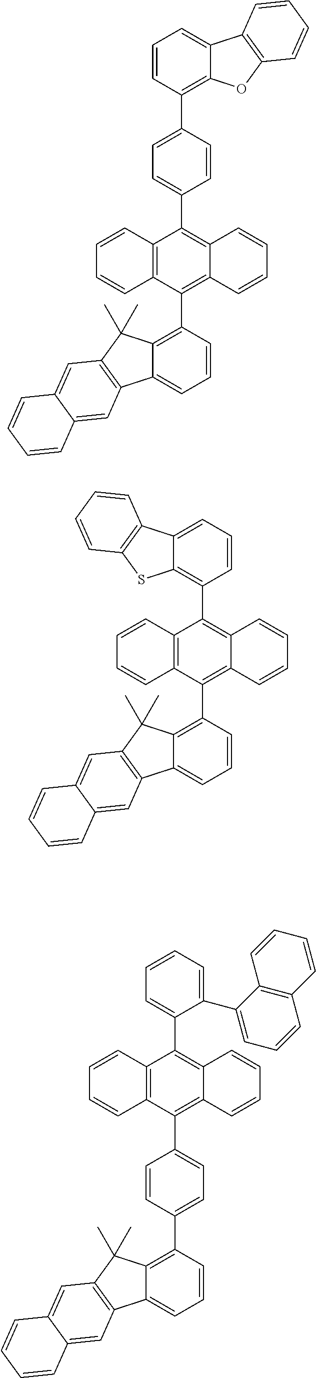

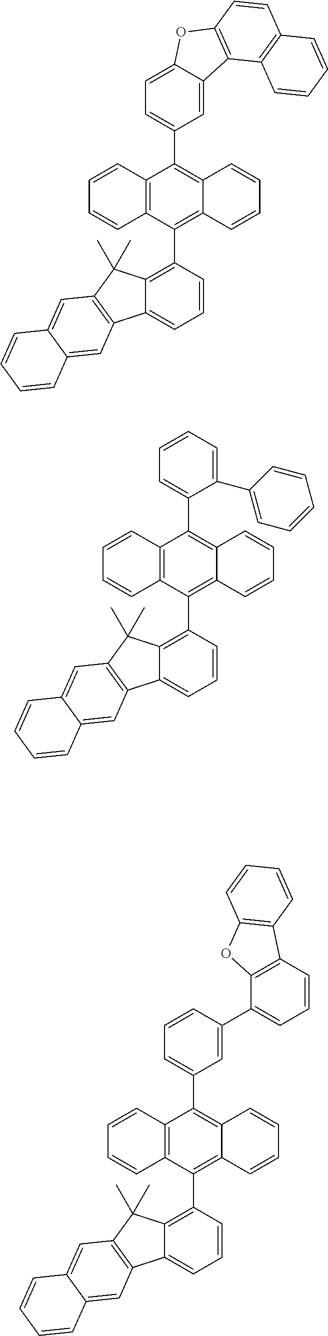

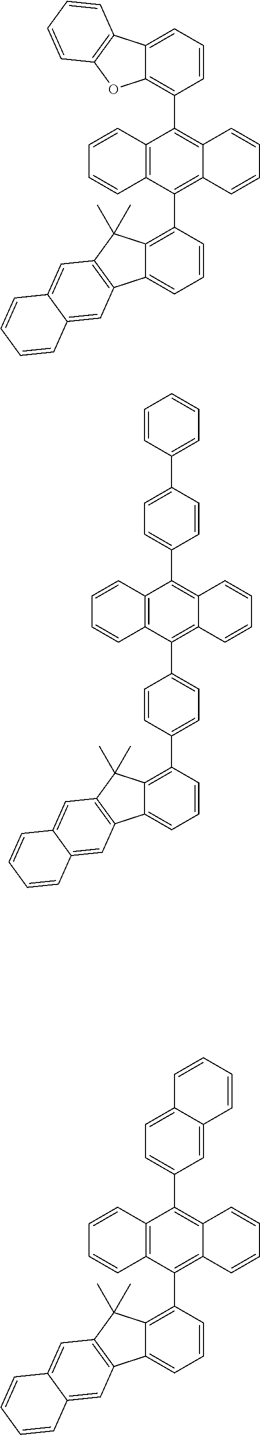

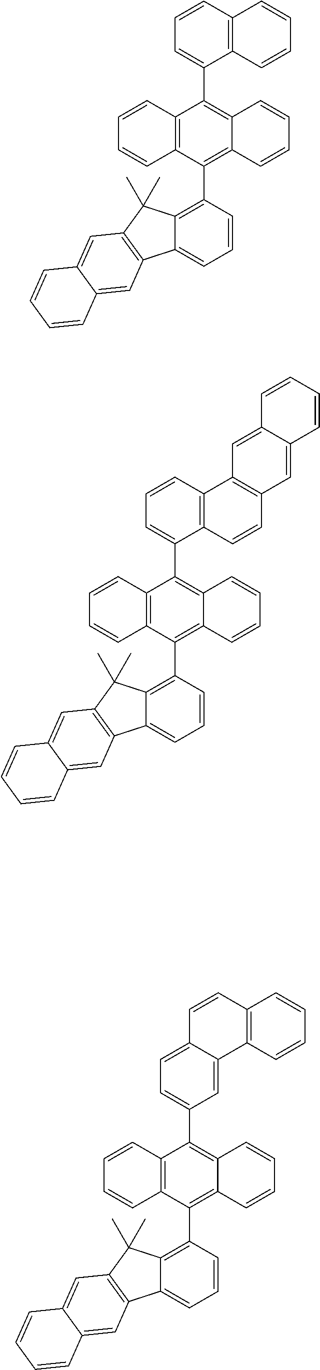

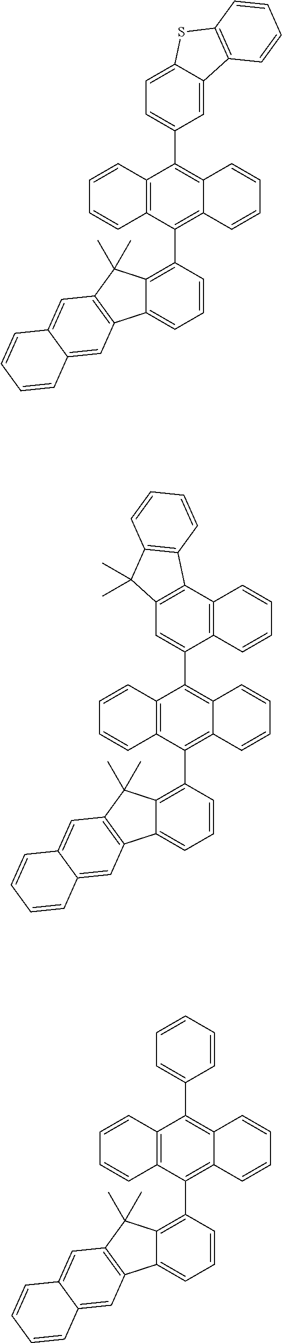

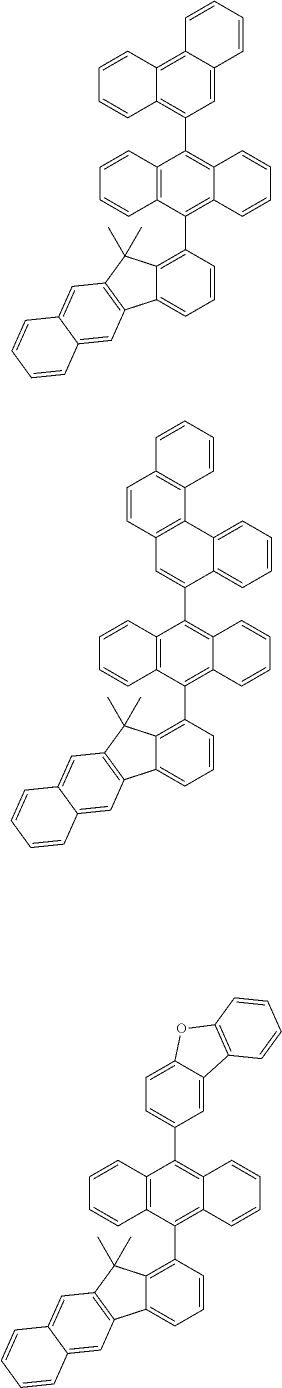

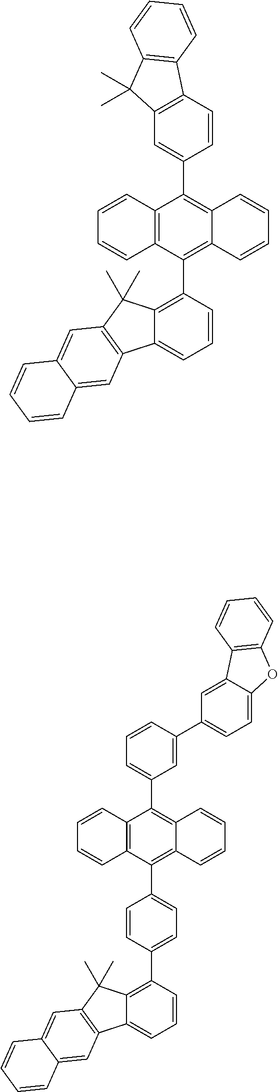

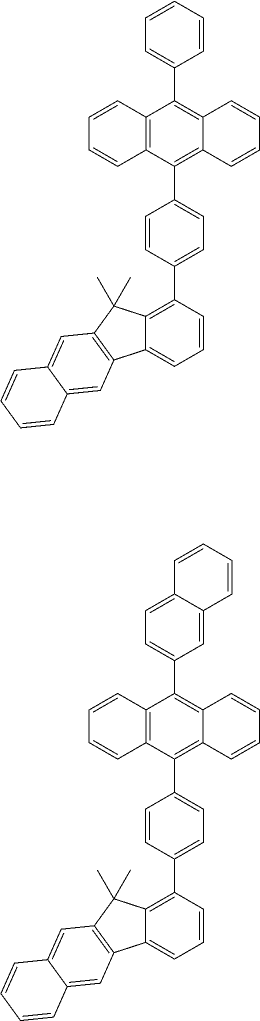

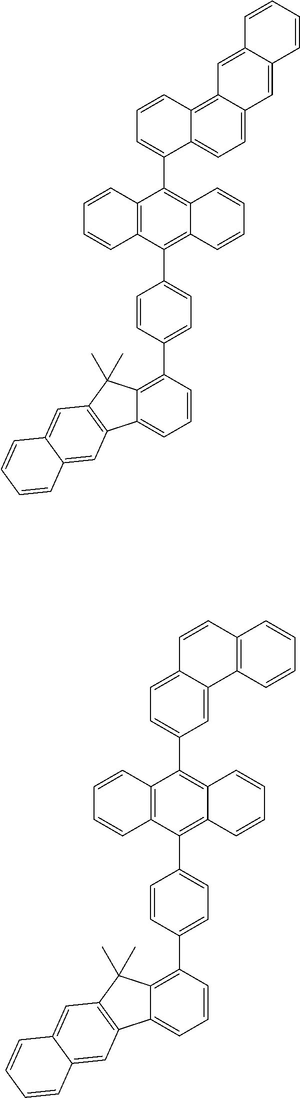

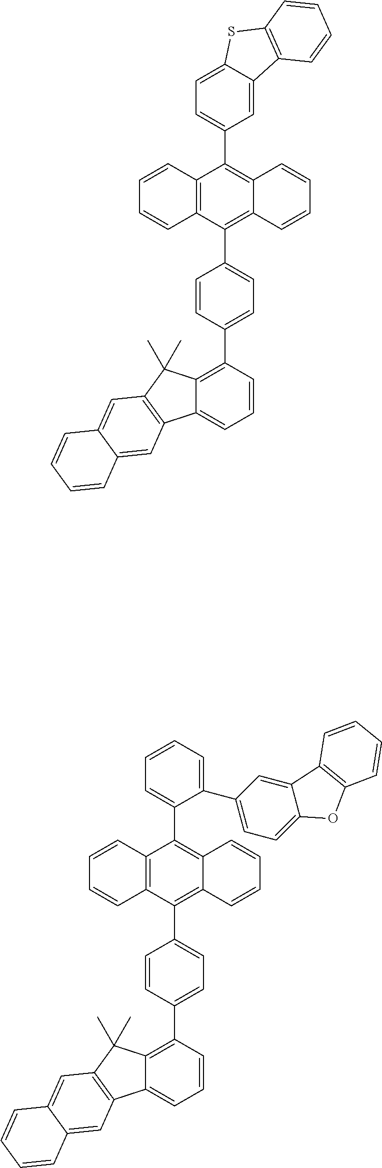 [SMILES: CC1(C)c2cc3ccccc3cc2-c2cccc(-c3ccc(-c4c5ccccc5c(-c5ccc6sc7ccccc7c6c5)c5ccccc45)cc3)c21 ; CC1(C)c2cc3ccccc3cc2-c2cccc(-c3ccc(-c4c5ccccc5c(-c5ccccc5-c5ccc6oc7ccccc7c6c5)c5ccccc45)cc3)c21]

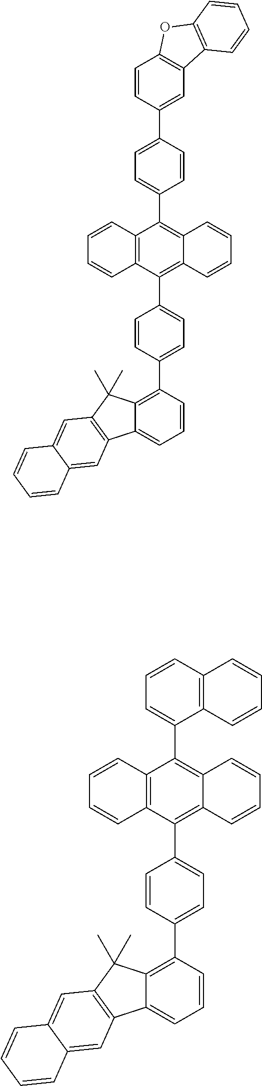

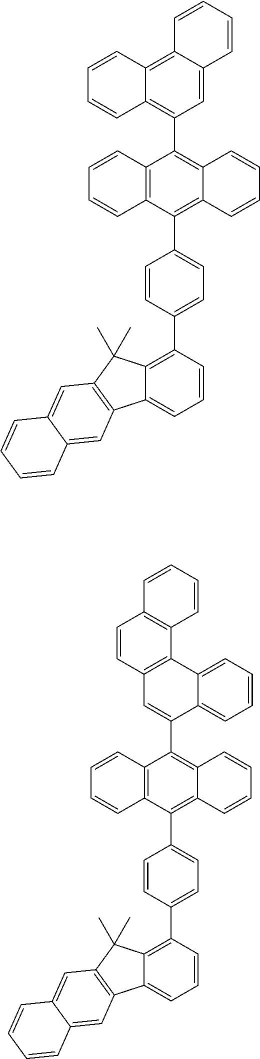

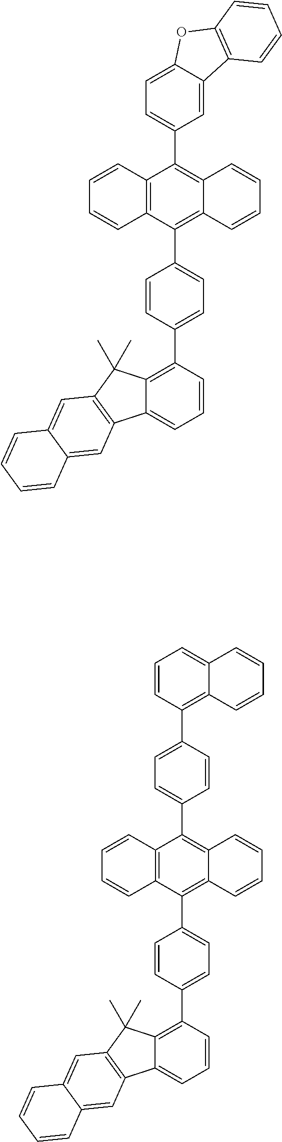

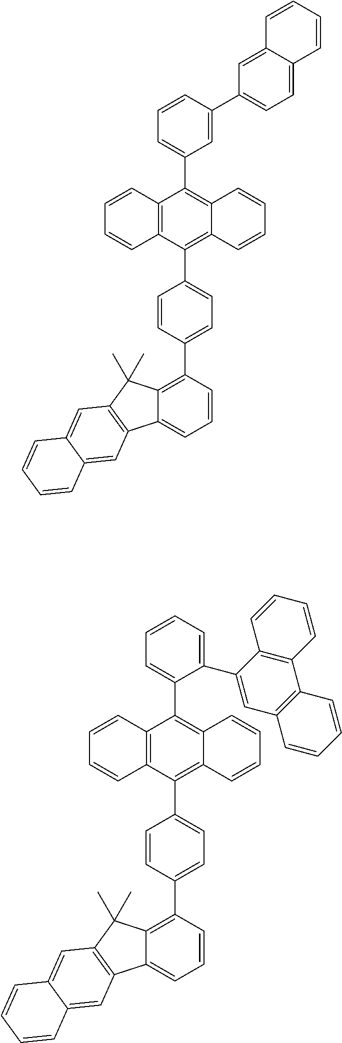

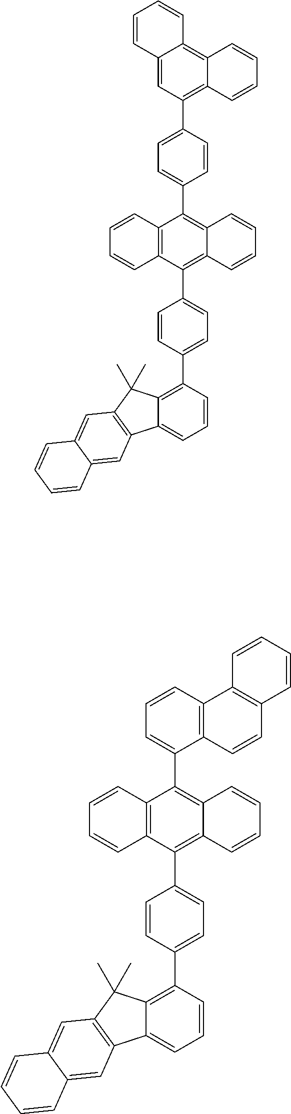

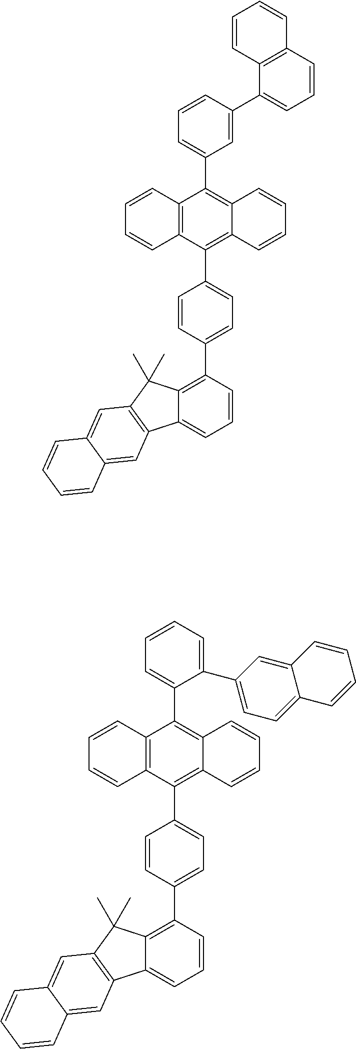

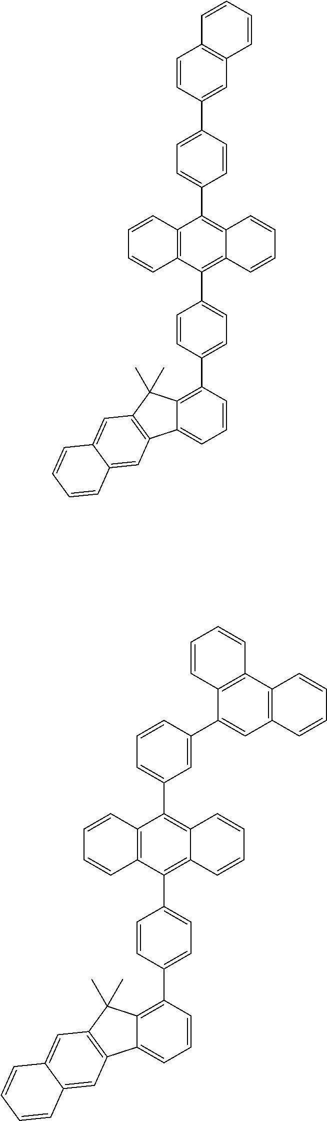

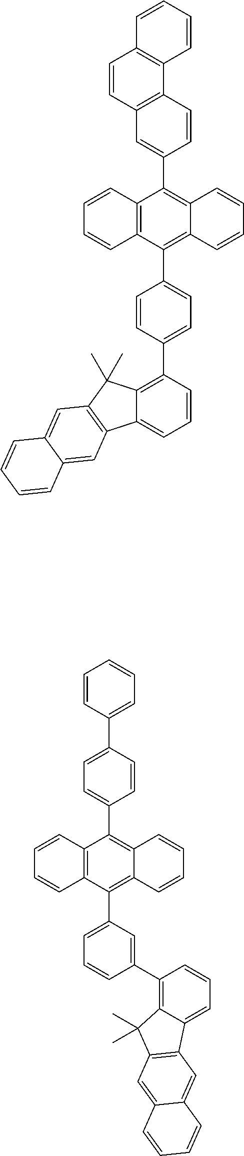

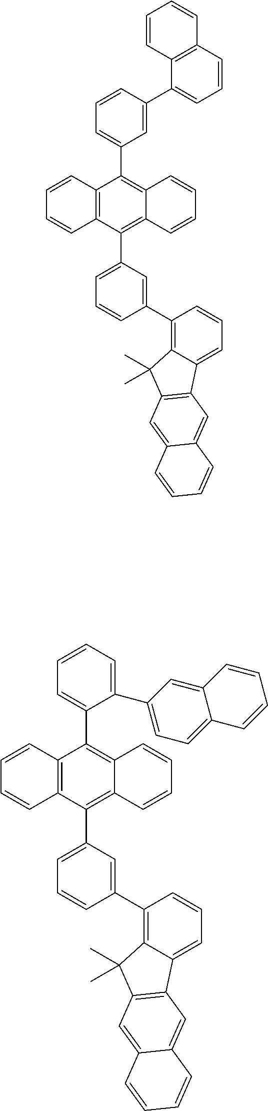

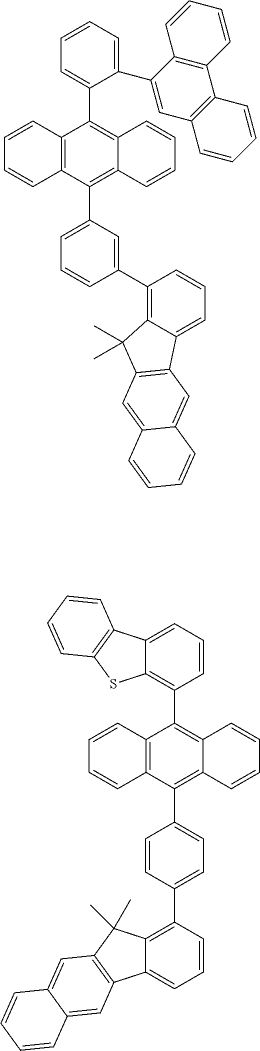

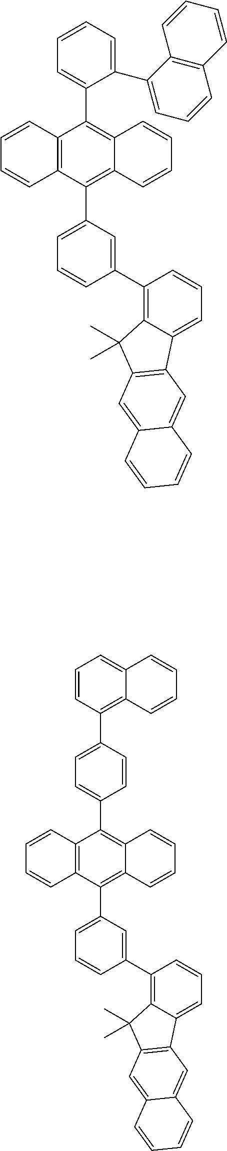

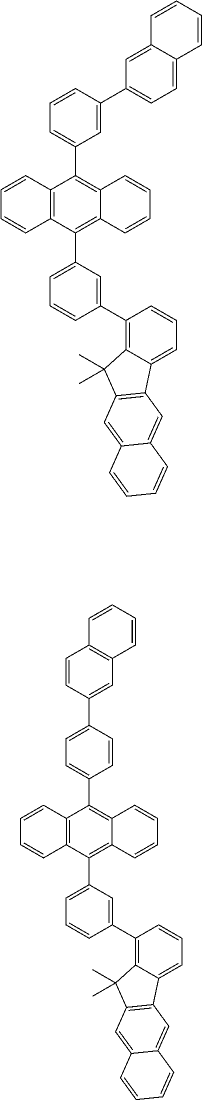

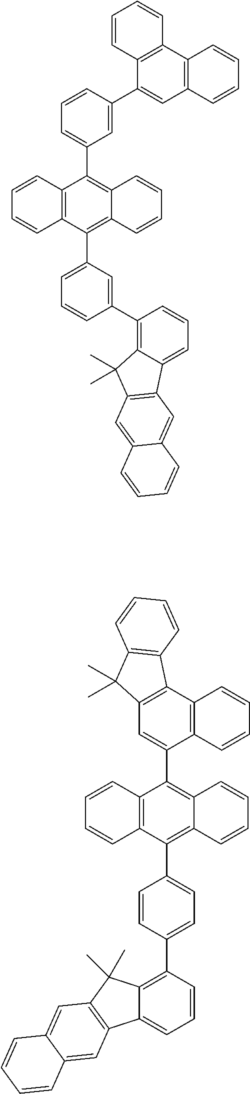

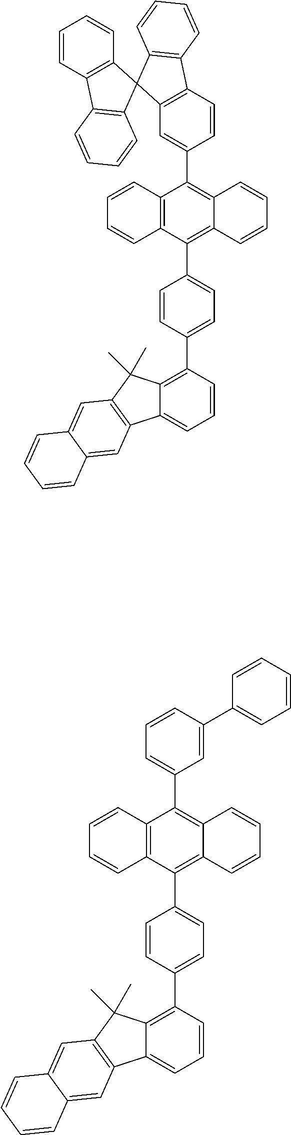

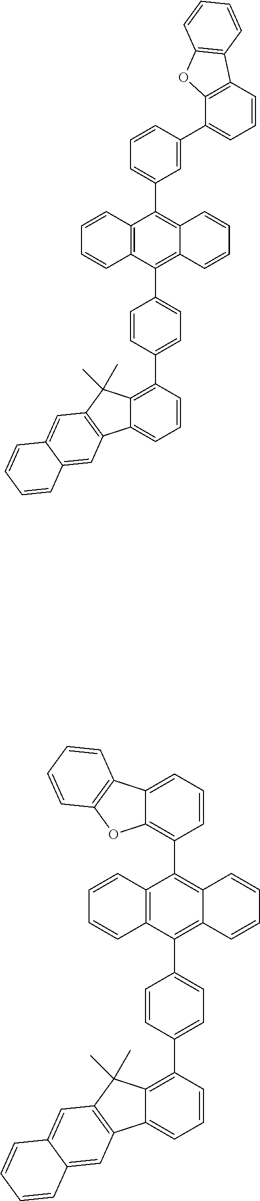 [SMILES: CC1(C)c2cc3ccccc3cc2-c2cccc(-c3ccc(-c4c5ccccc5c(-c5cccc(-c6cccc7c6oc6ccccc67)c5)c5ccccc45)cc3)c21 ; CC1(C)c2cc3ccccc3cc2-c2cccc(-c3ccc(-c4c5ccccc5c(-c5cccc6c5oc5ccccc56)c5ccccc45)cc3)c21]

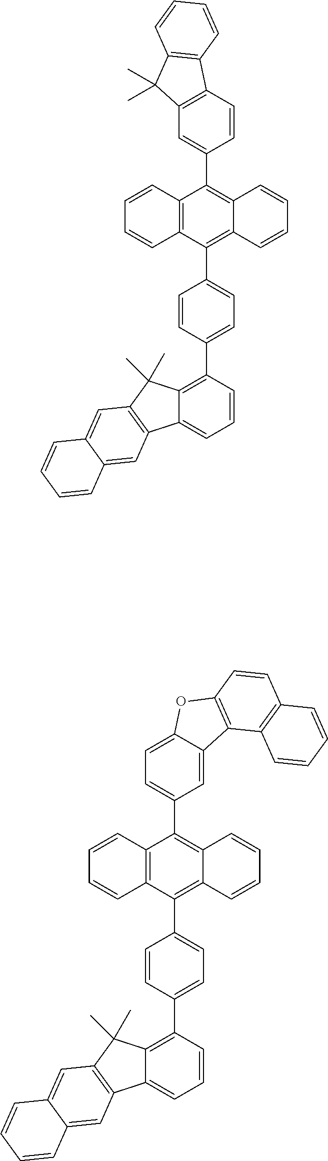

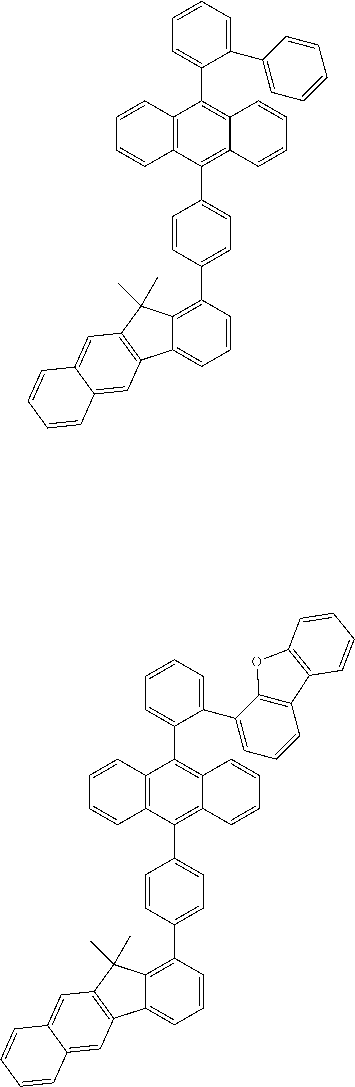

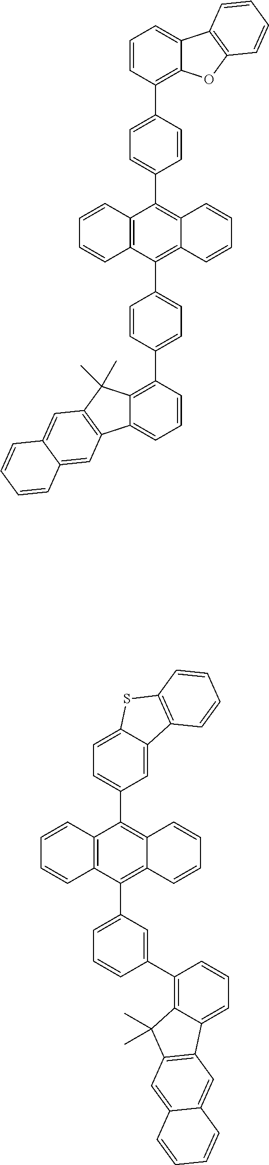 [SMILES: CC1(C)c2cc3ccccc3cc2-c2cccc(-c3ccc(-c4c5ccccc5c(-c5ccc(-c6cccc7c6oc6ccccc67)cc5)c5ccccc45)cc3)c21 ; CC1(C)c2cc3ccccc3cc2-c2cccc(-c3cccc(-c4c5ccccc5c(-c5ccc6sc7ccccc7c6c5)c5ccccc45)c3)c21]

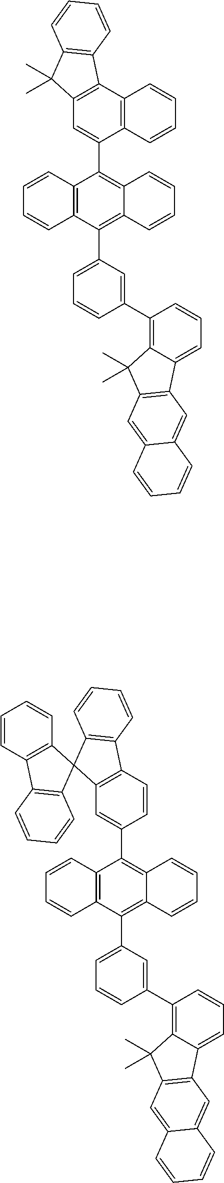

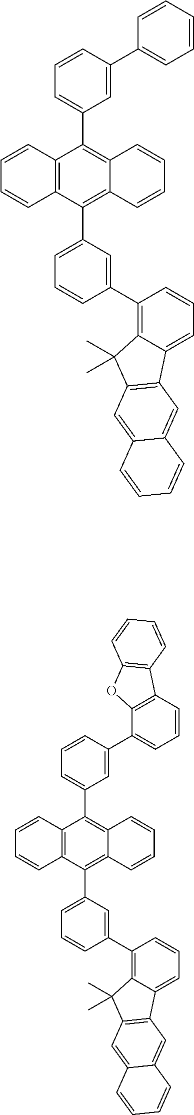

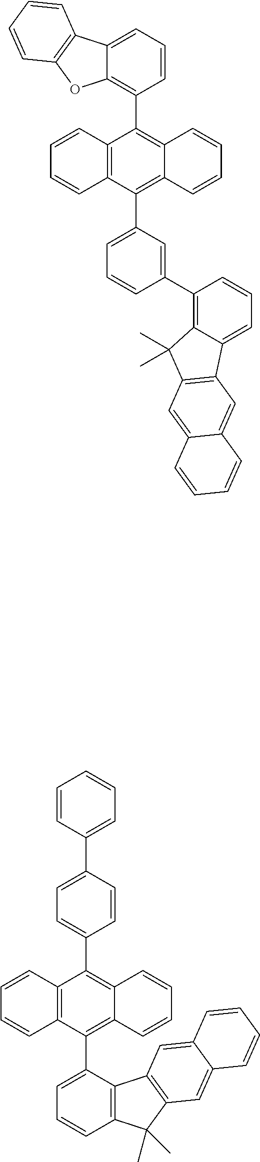

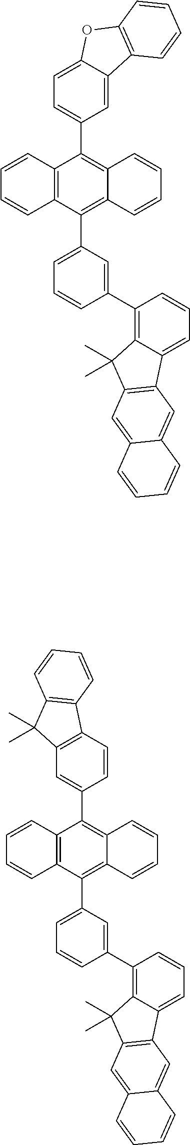

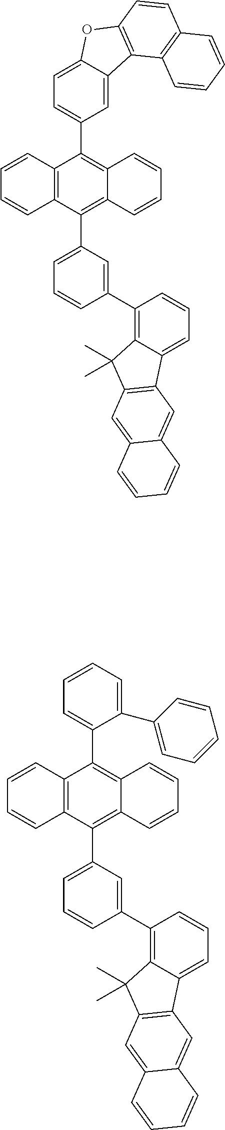

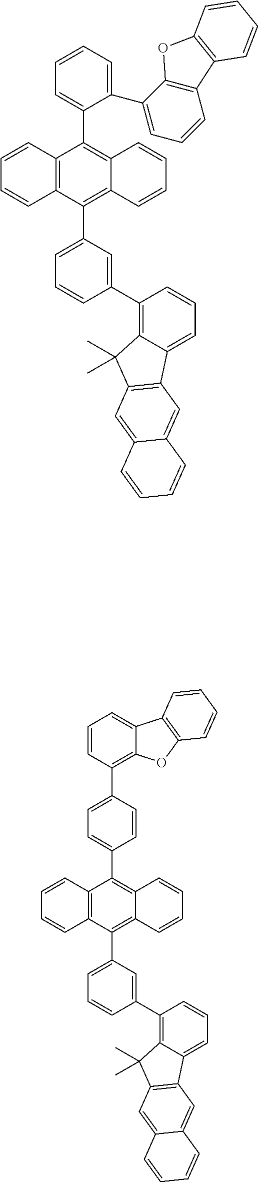 [SMILES: CC1(C)c2cc3ccccc3cc2-c2cccc(-c3cccc(-c4c5ccccc5c(-c5ccccc5-c5cccc6c5oc5ccccc56)c5ccccc45)c3)c21 ; CC1(C)c2cc3ccccc3cc2-c2cccc(-c3cccc(-c4c5ccccc5c(-c5ccc(-c6cccc7c6oc6ccccc67)cc5)c5ccccc45)c3)c21]

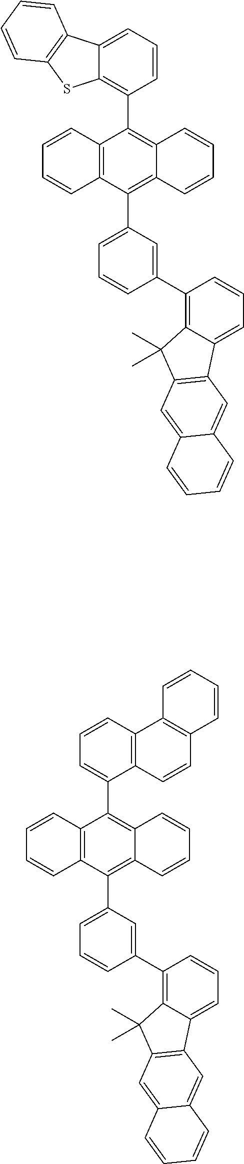

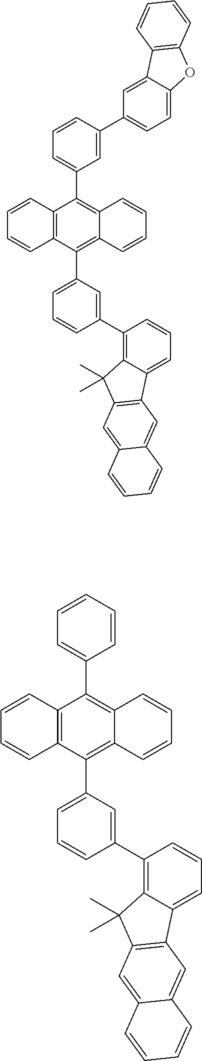

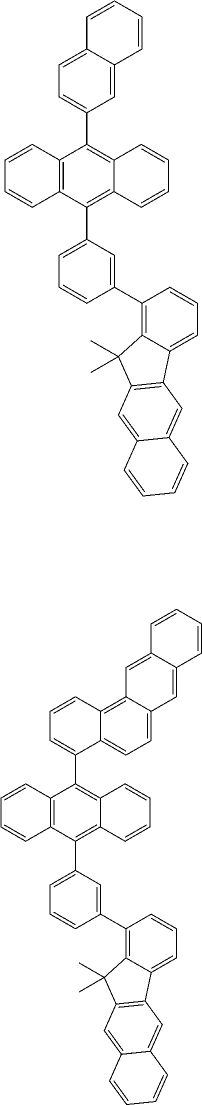

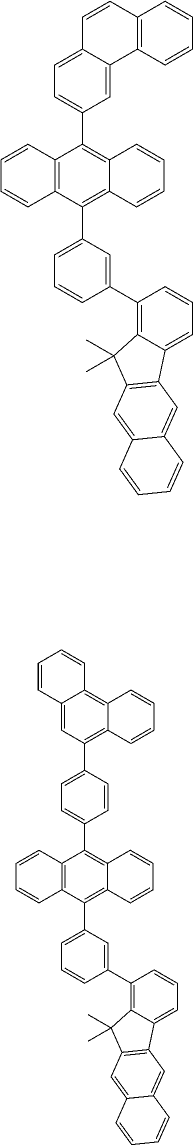

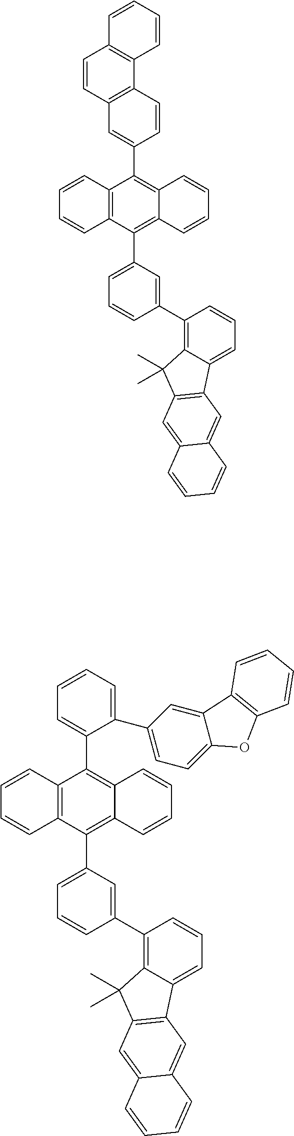

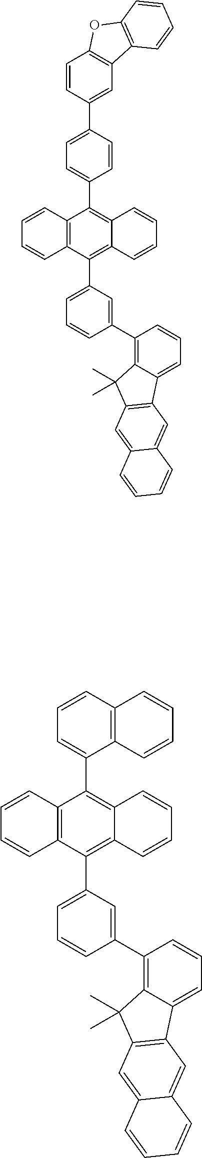

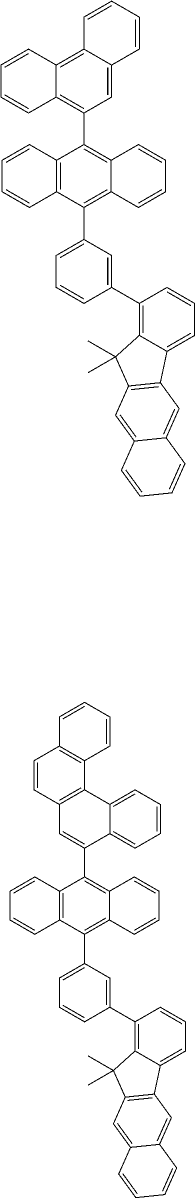

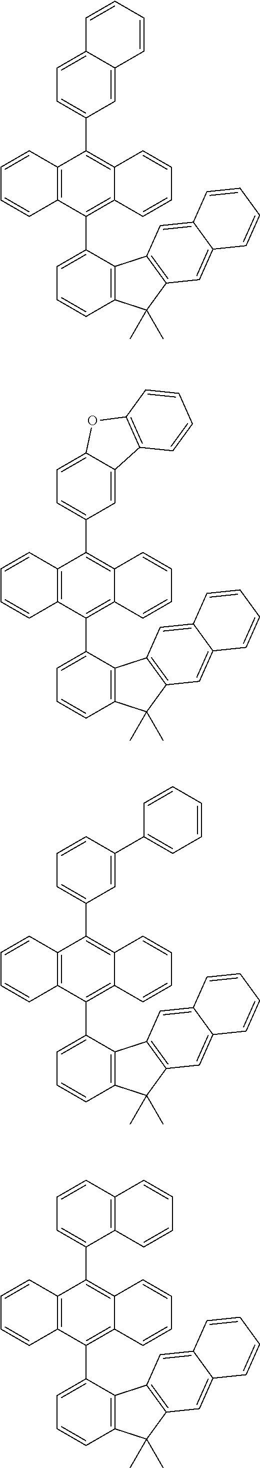

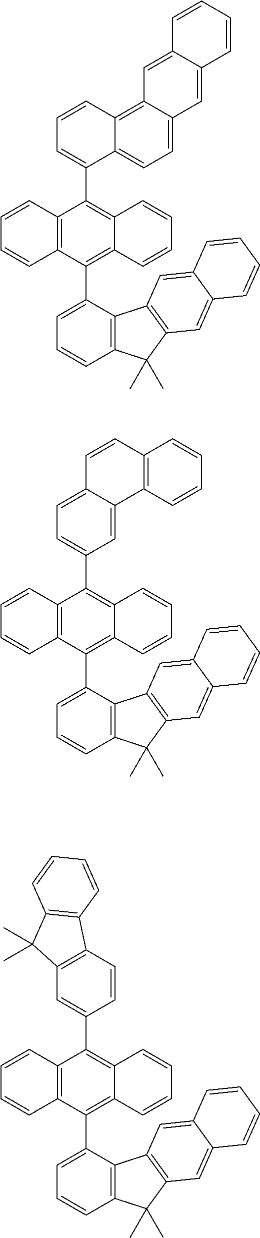

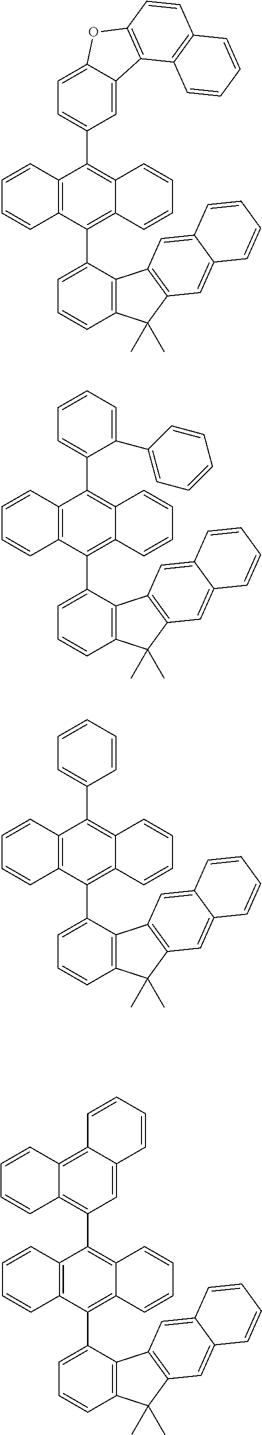

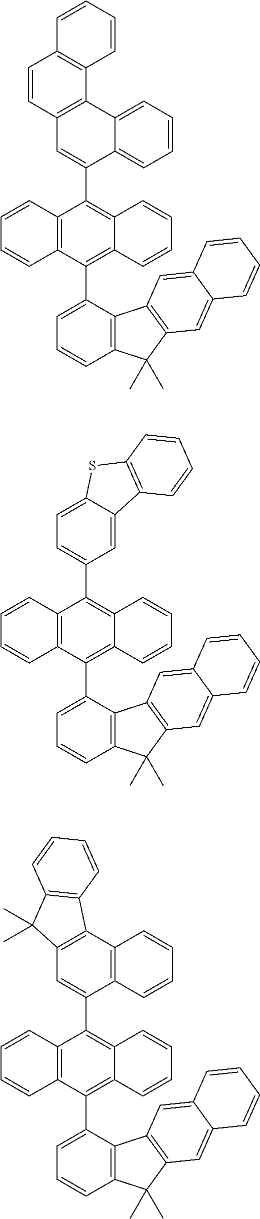

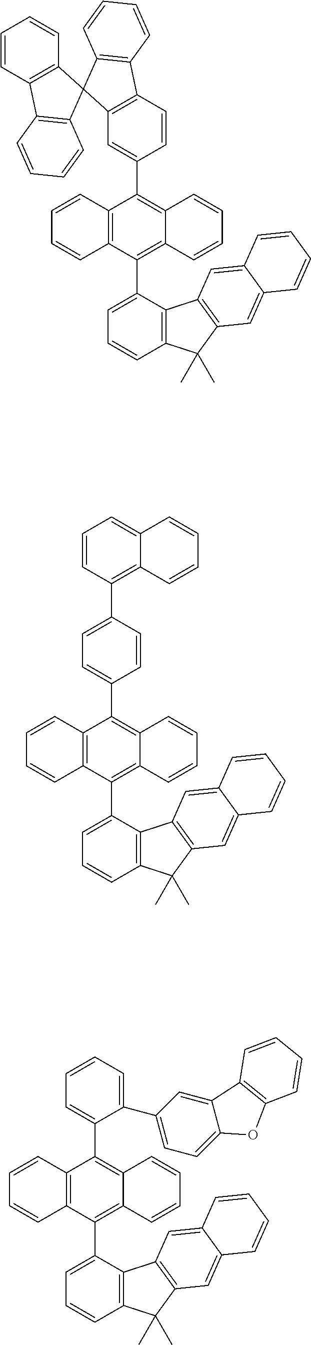

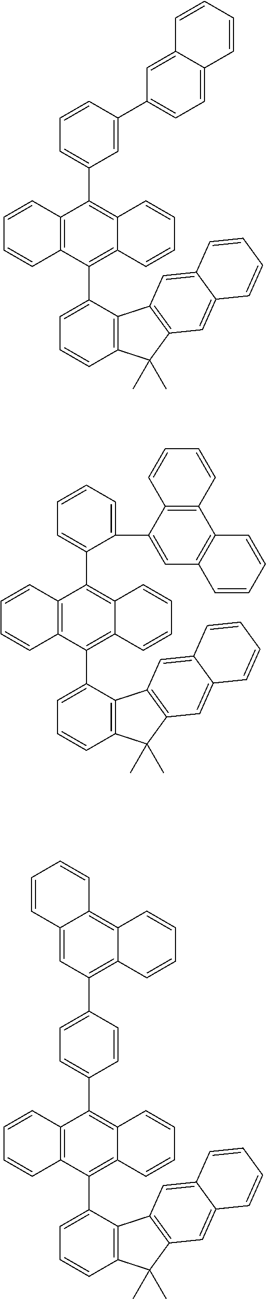

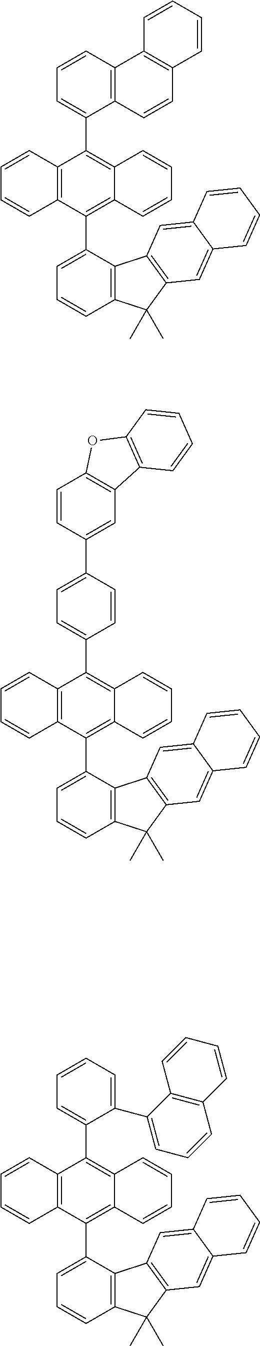

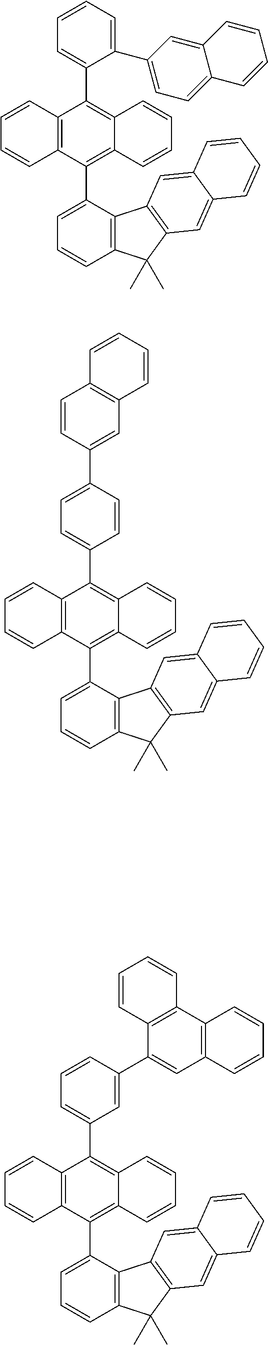

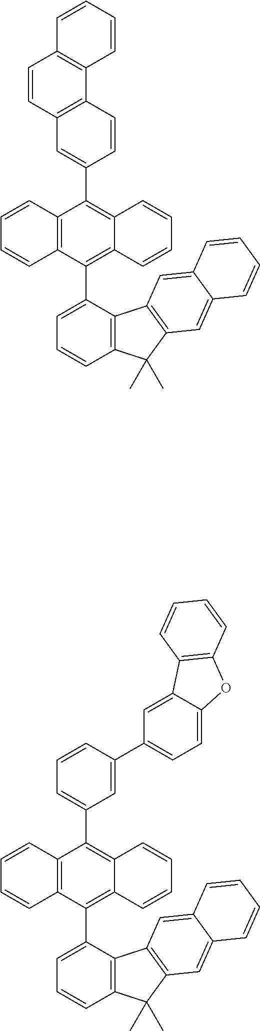

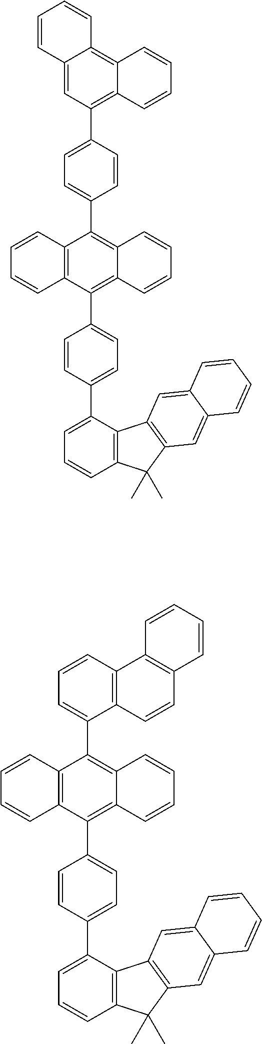

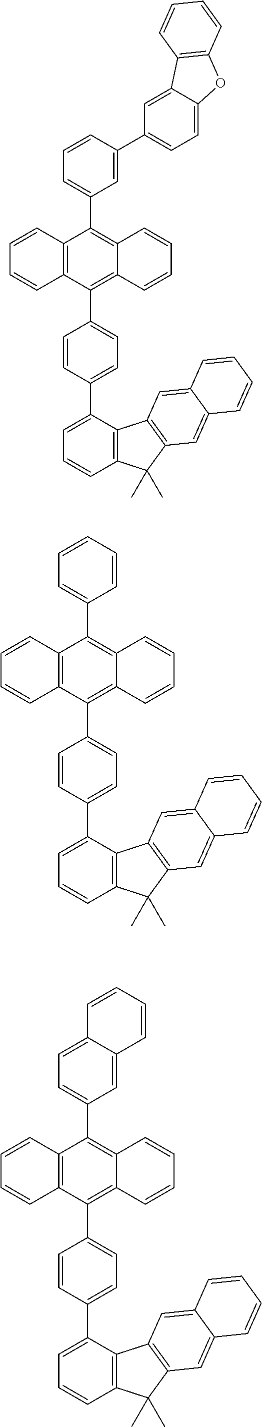

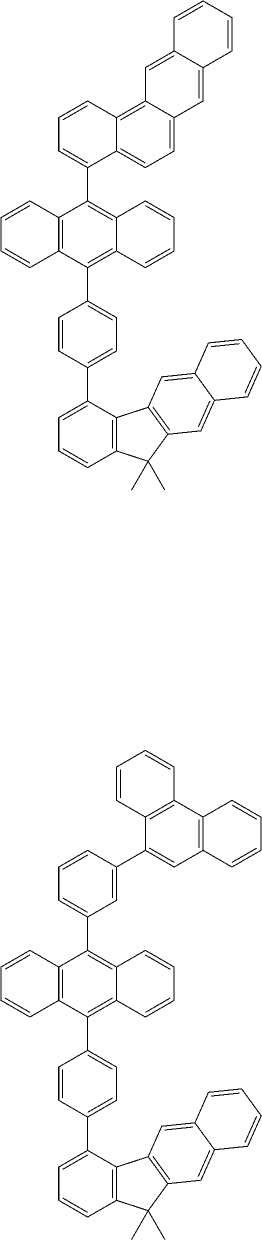

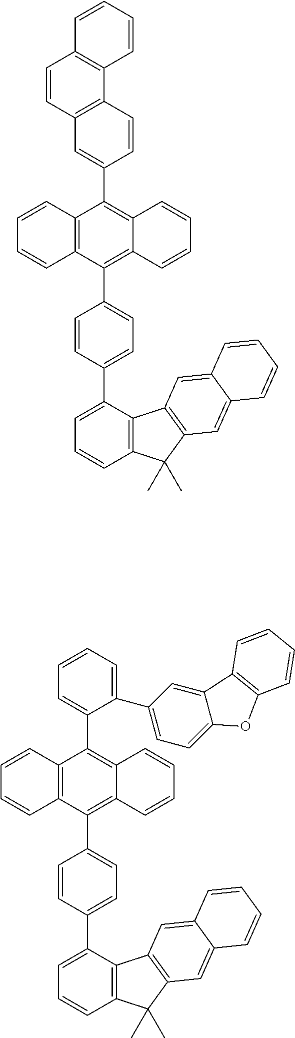

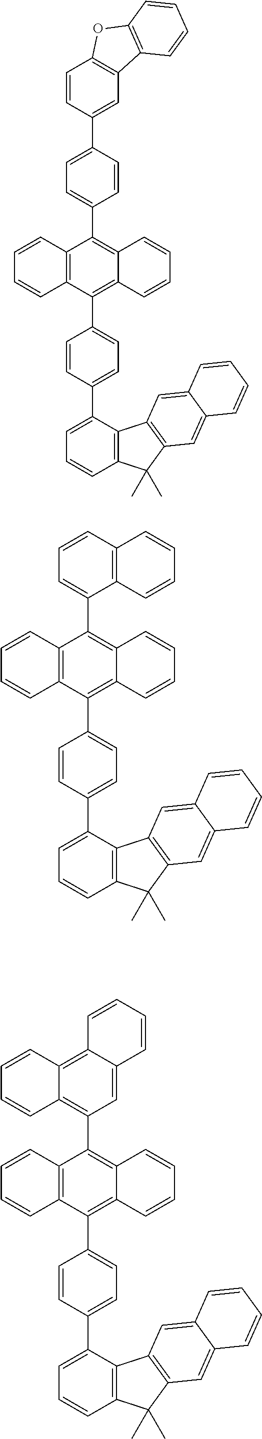

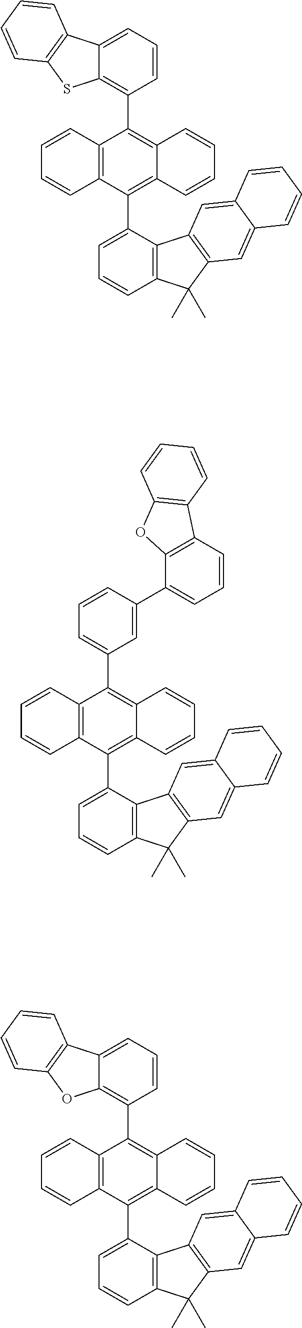 [SMILES: CC1(C)c2cc3ccccc3cc2-c2c(-c3c4ccccc4c(-c4cccc5c4sc4ccccc45)c4ccccc34)cccc21 ; CC1(C)c2cc3ccccc3cc2-c2c(-c3c4ccccc4c(-c4cccc(-c5cccc6c5oc5ccccc56)c4)c4ccccc34)cccc21 ; CC1(C)c2cc3ccccc3cc2-c2c(-c3c4ccccc4c(-c4cccc5c4oc4ccccc45)c4ccccc34)cccc21]

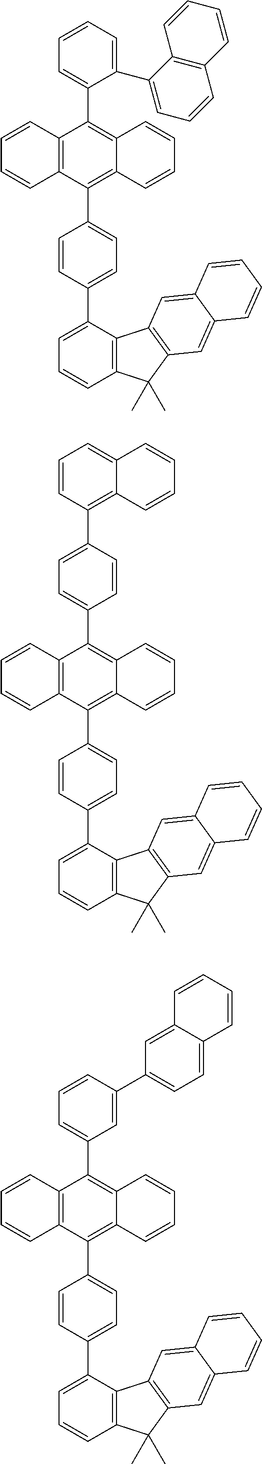

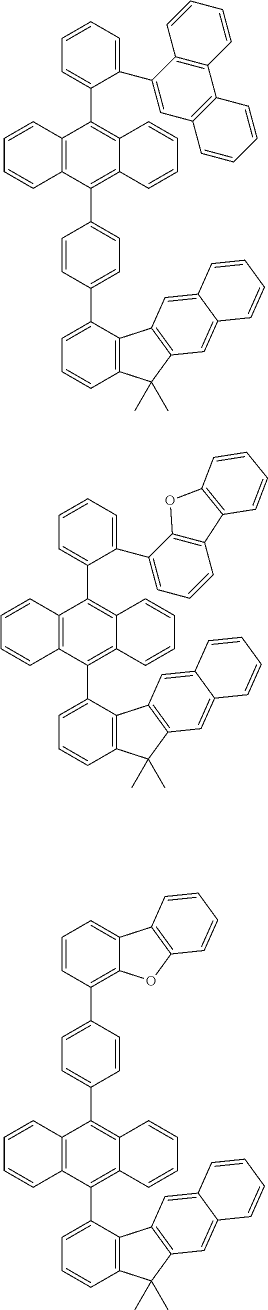

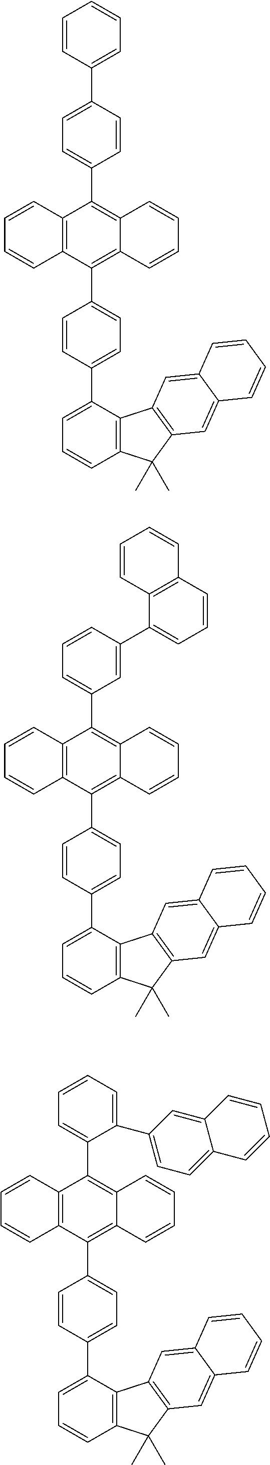

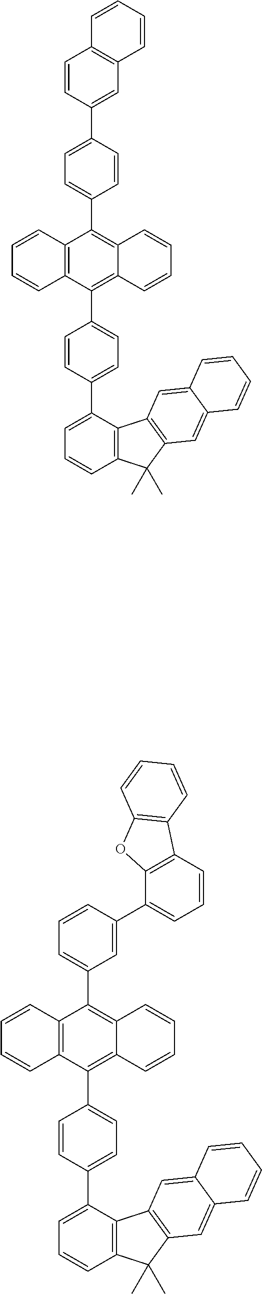

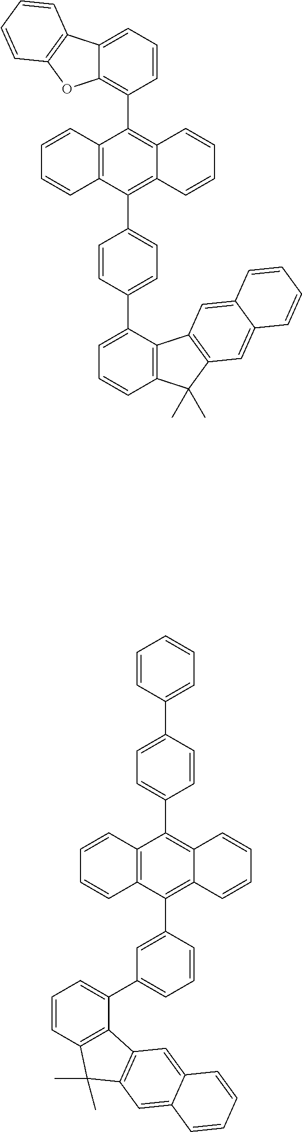

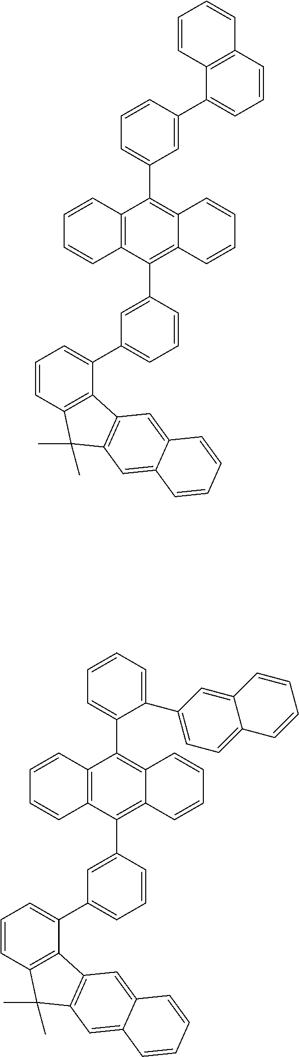

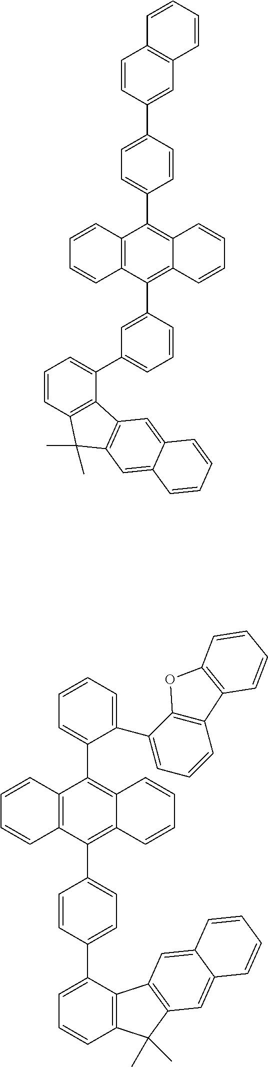

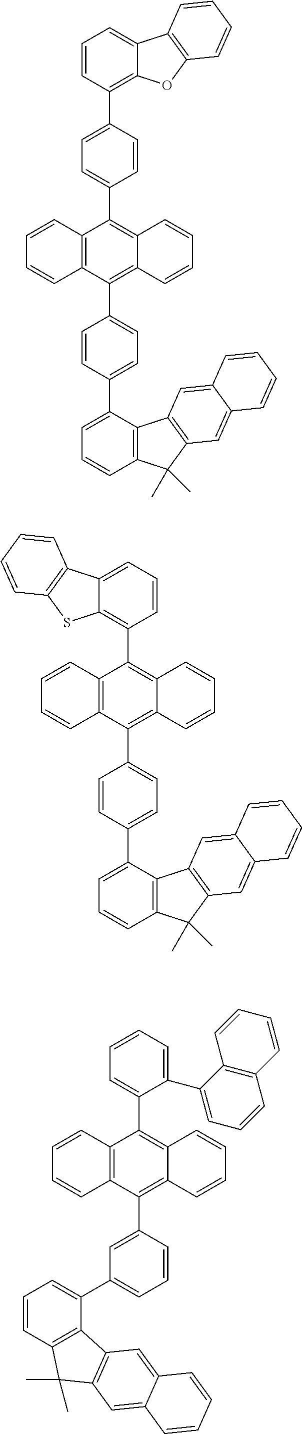

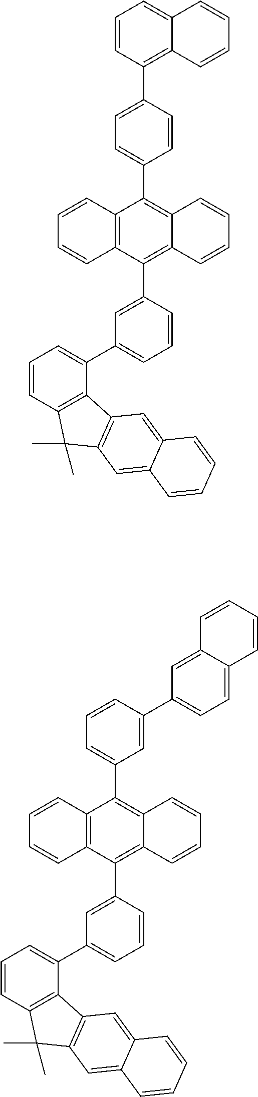

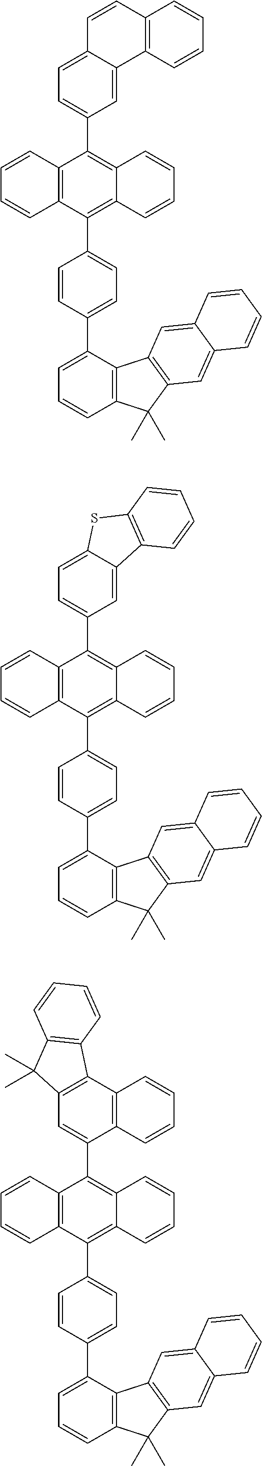

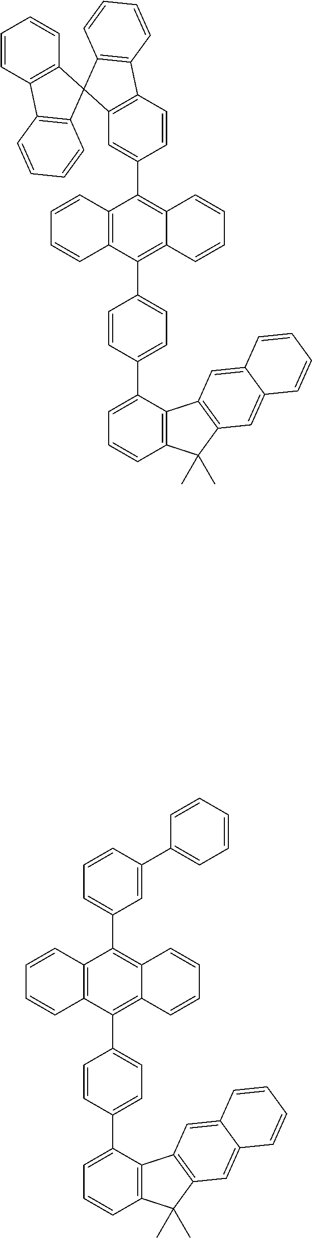

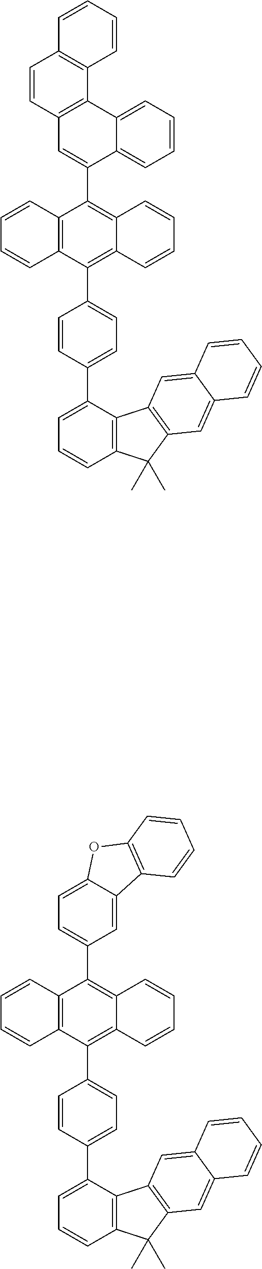

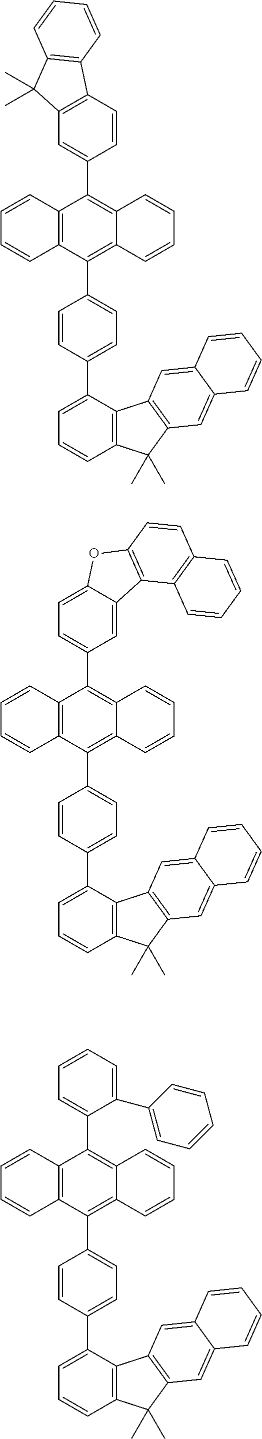

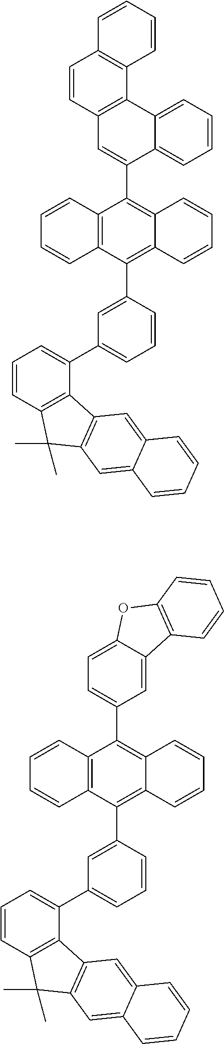

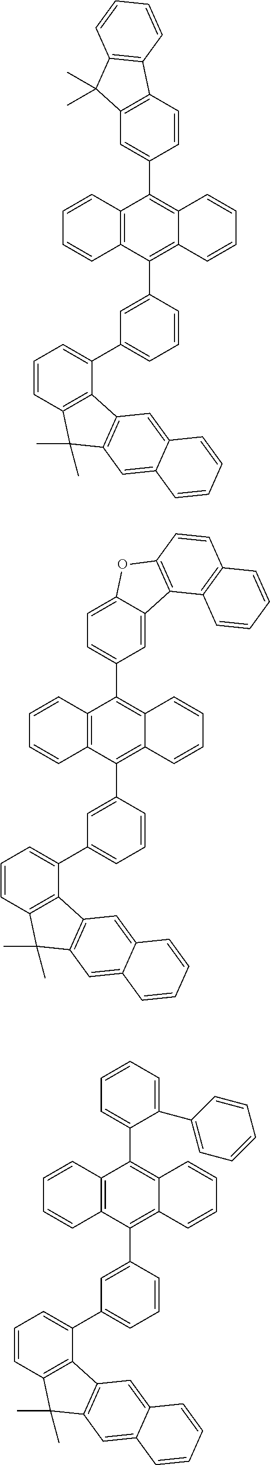

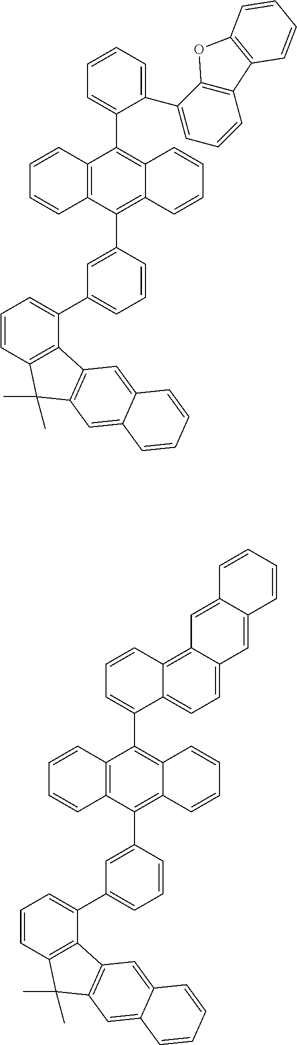

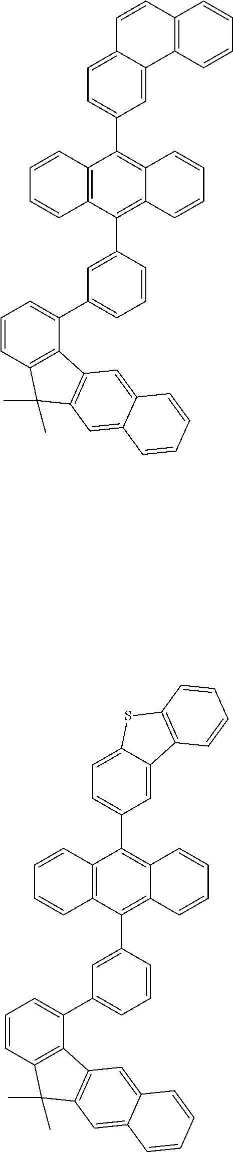

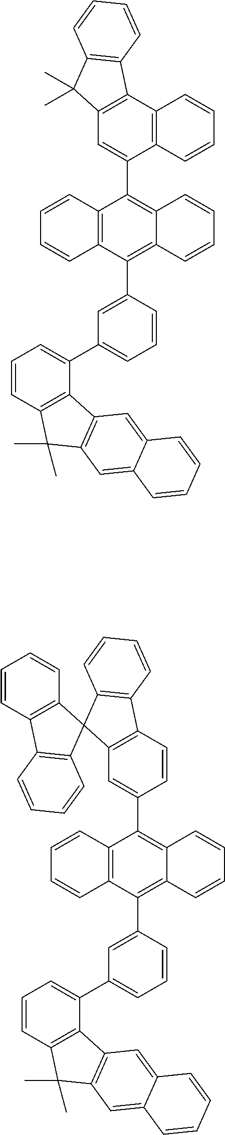

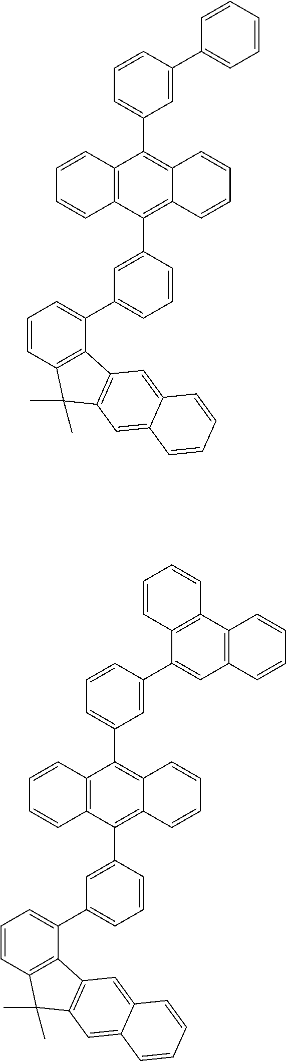

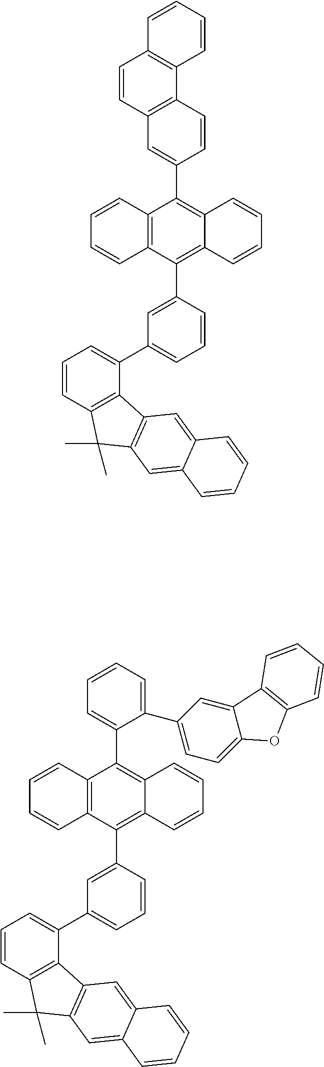

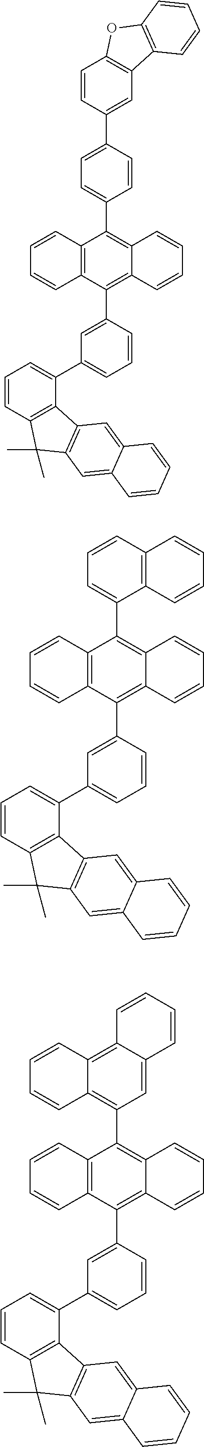

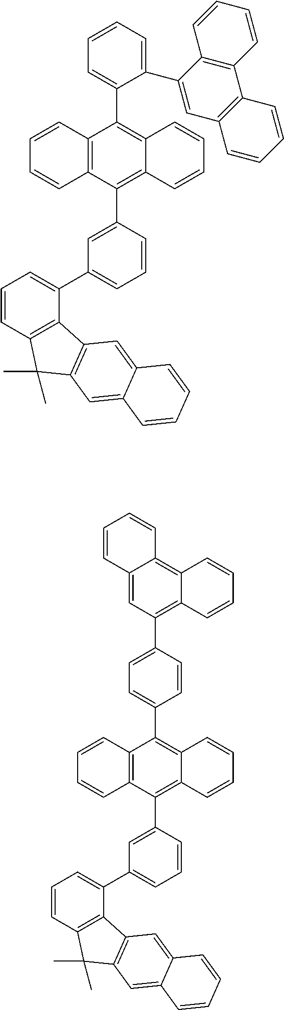

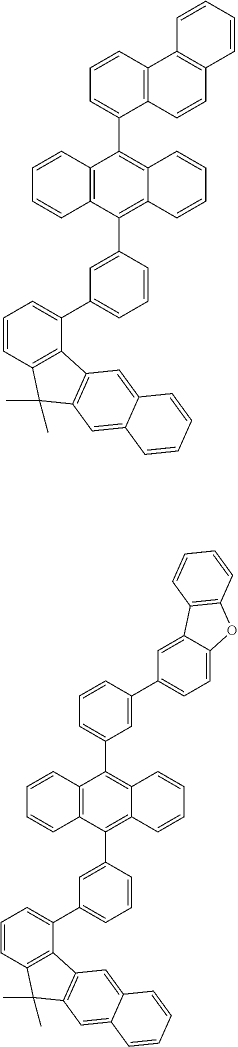

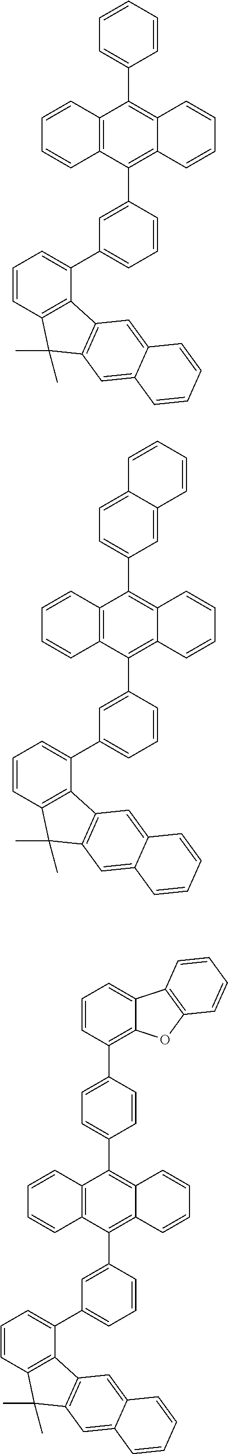

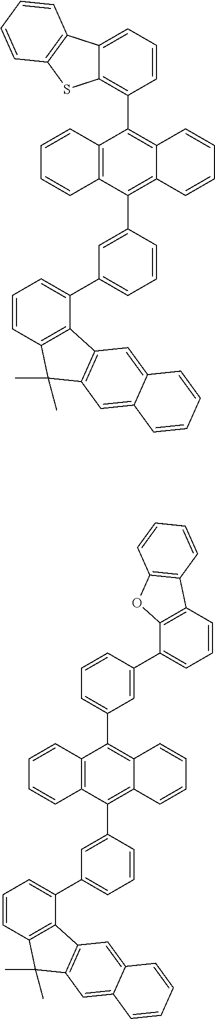 [SMILES: CC1(C)c2cc3ccccc3cc2-c2c(-c3cccc(-c4c5ccccc5c(-c5cccc6c5sc5ccccc56)c5ccccc45)c3)cccc21 ; CC1(C)c2cc3ccccc3cc2-c2c(-c3cccc(-c4c5ccccc5c(-c5cccc(-c6cccc7c6oc6ccccc67)c5)c5ccccc45)c3)cccc21]

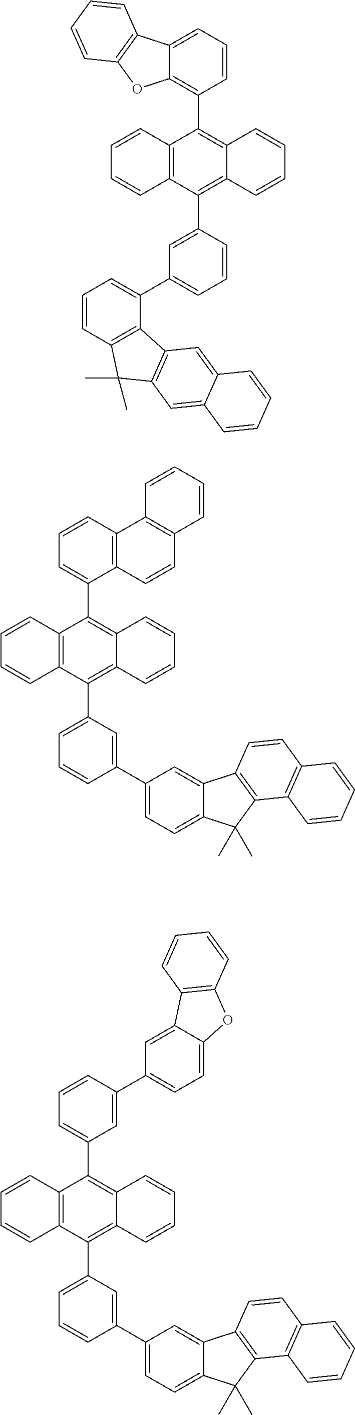

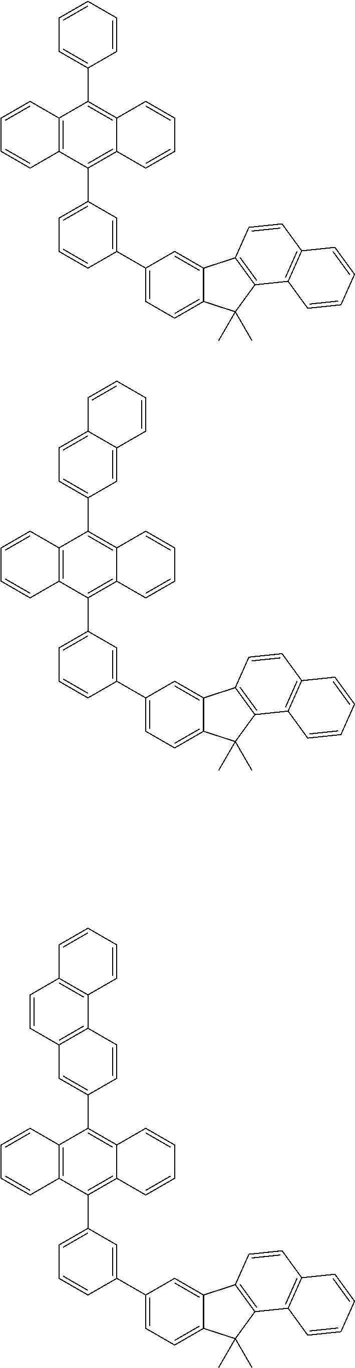

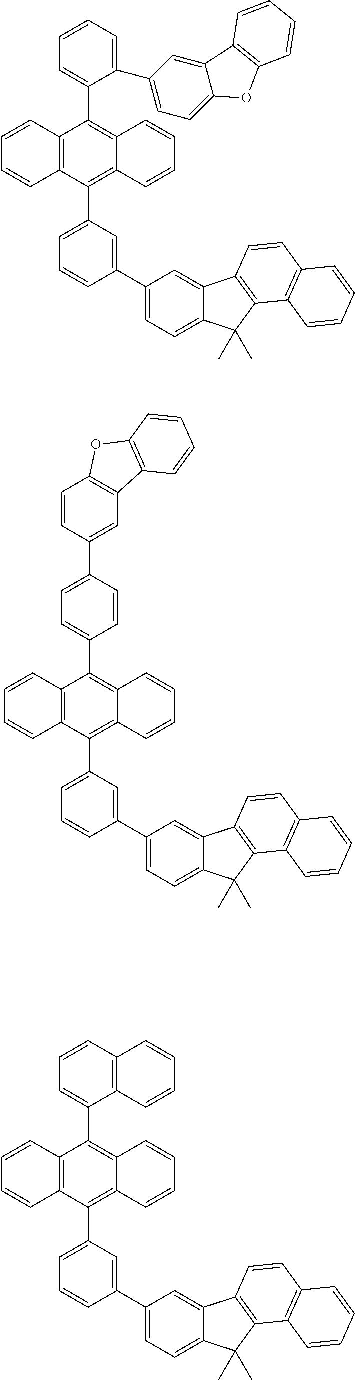

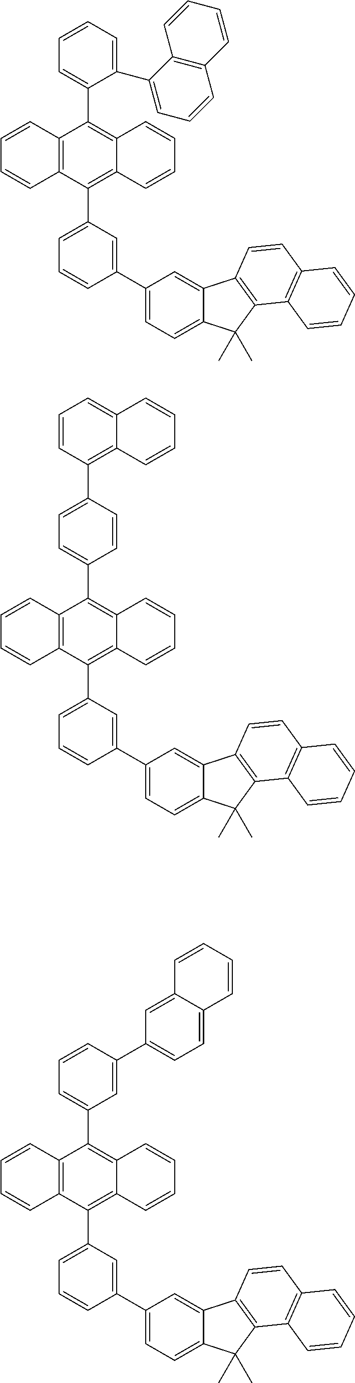

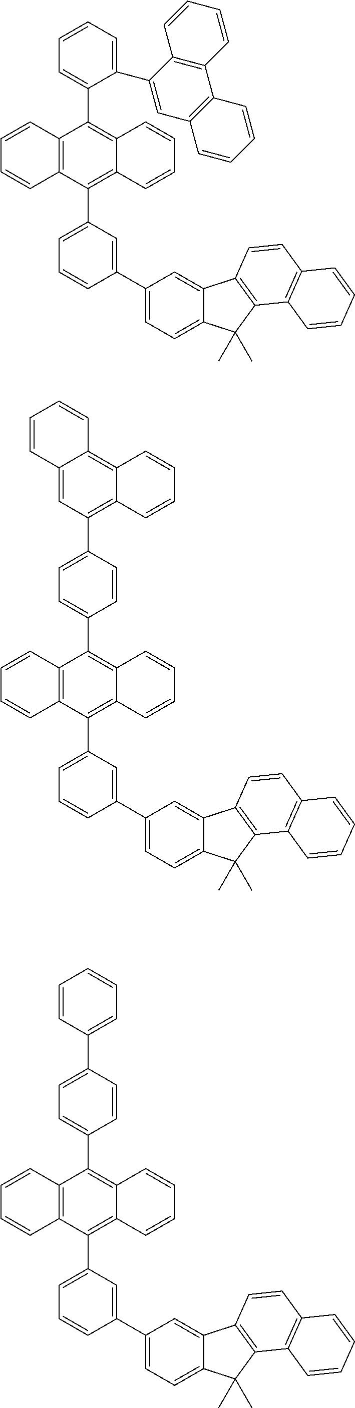

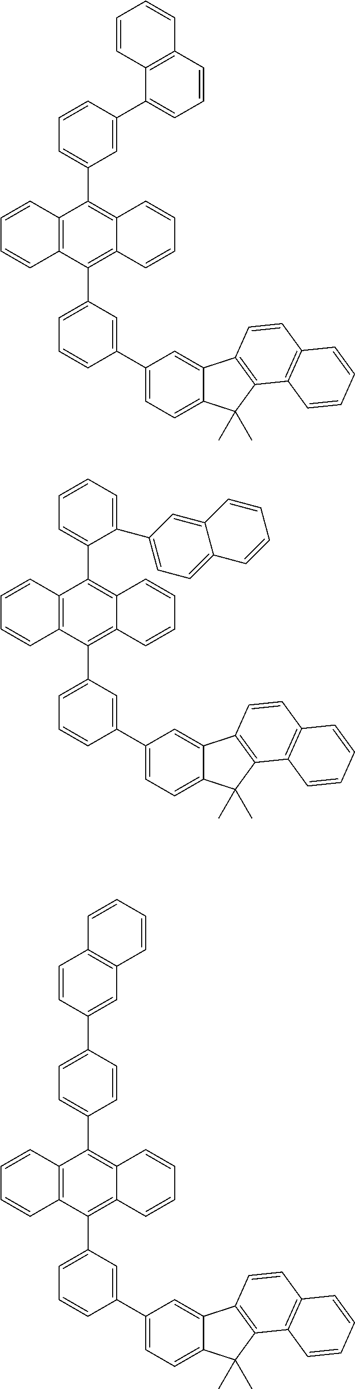

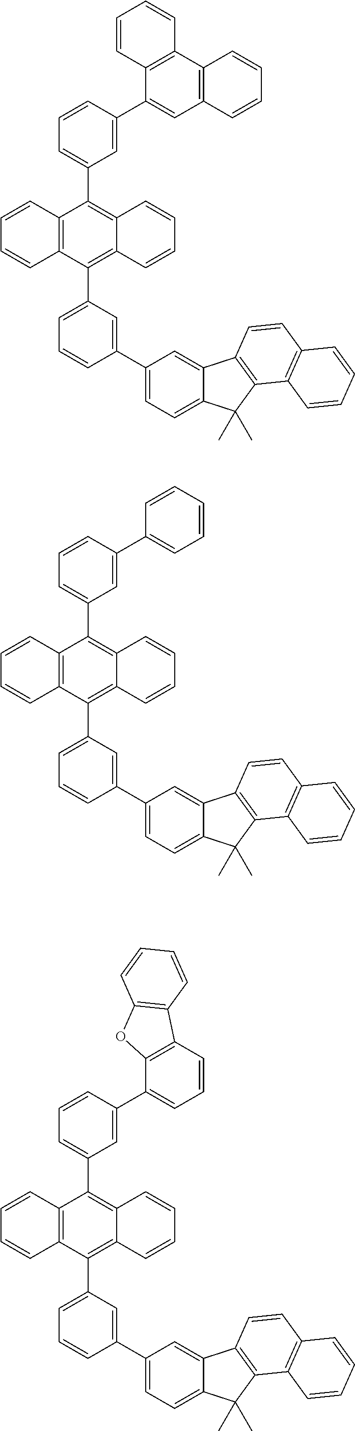

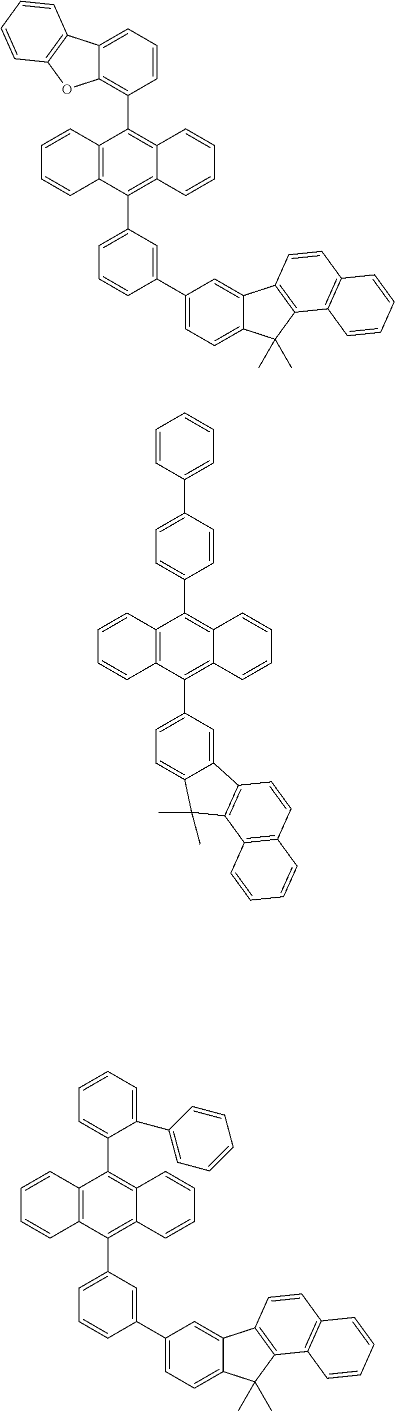

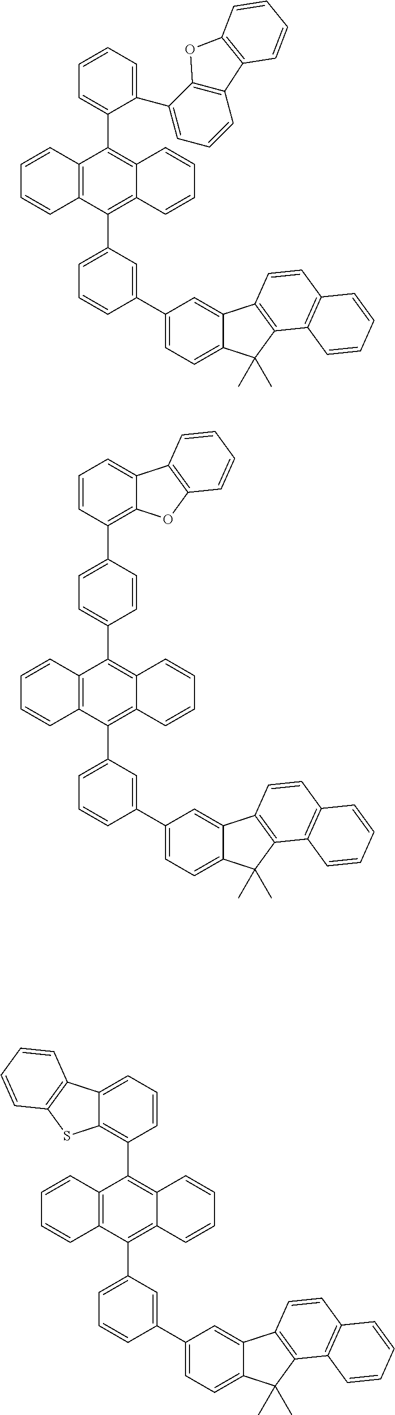 [SMILES: CC1(C)c2ccc(-c3cccc(-c4c5ccccc5c(-c5ccccc5-c5cccc6c5oc5ccccc56)c5ccccc45)c3)cc2-c2ccc3ccccc3c21 ; CC1(C)c2ccc(-c3cccc(-c4c5ccccc5c(-c5ccc(-c6cccc7c6oc6ccccc67)cc5)c5ccccc45)c3)cc2-c2ccc3ccccc3c21 ; CC1(C)c2ccc(-c3cccc(-c4c5ccccc5c(-c5cccc6c5sc5ccccc56)c5ccccc45)c3)cc2-c2ccc3ccccc3c21]

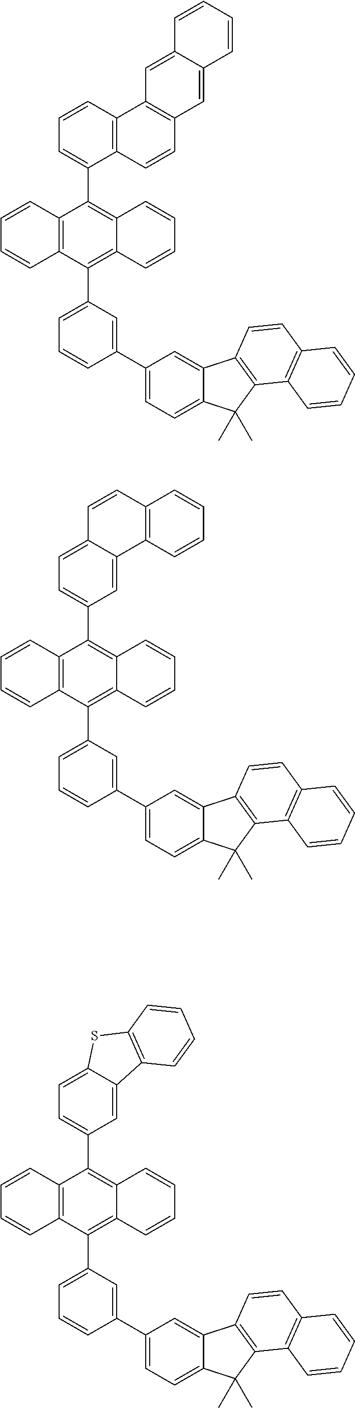

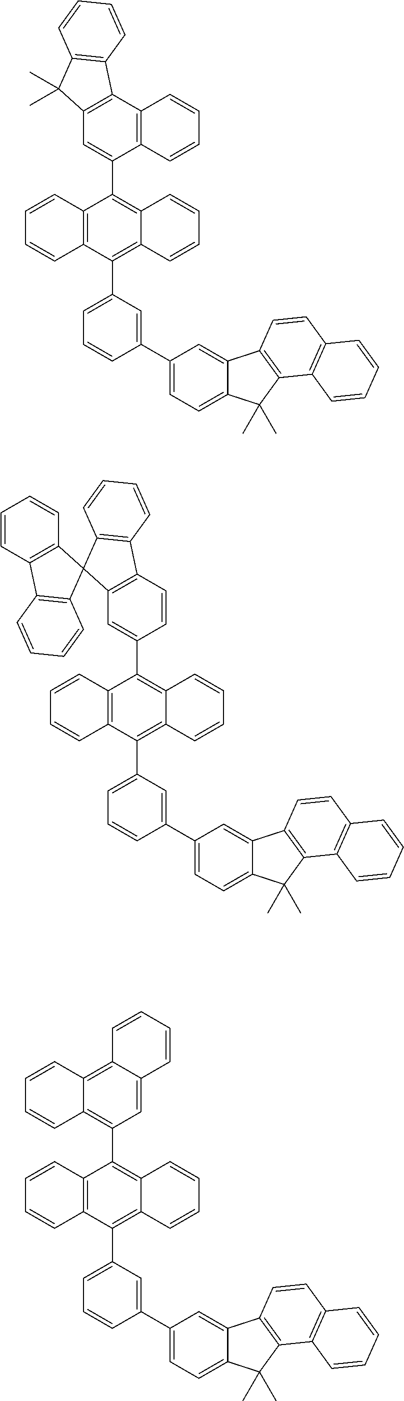

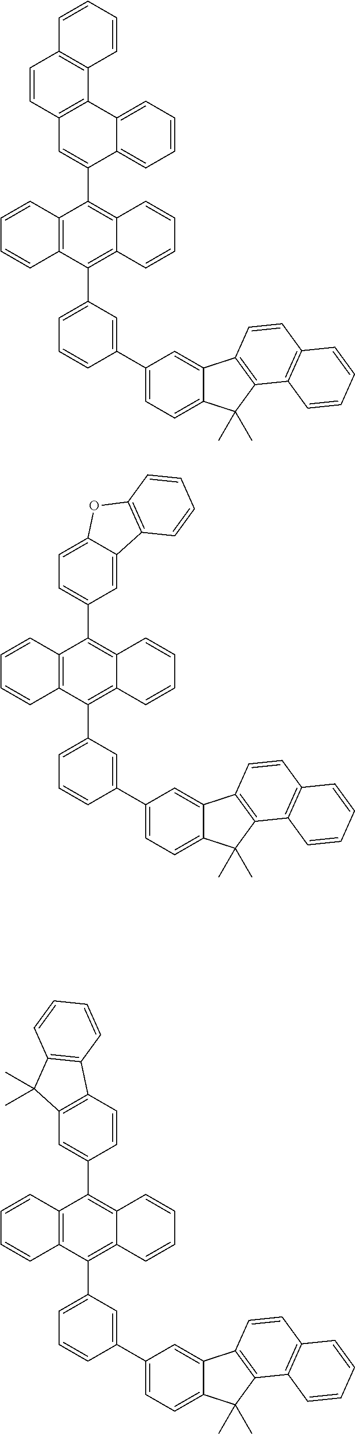

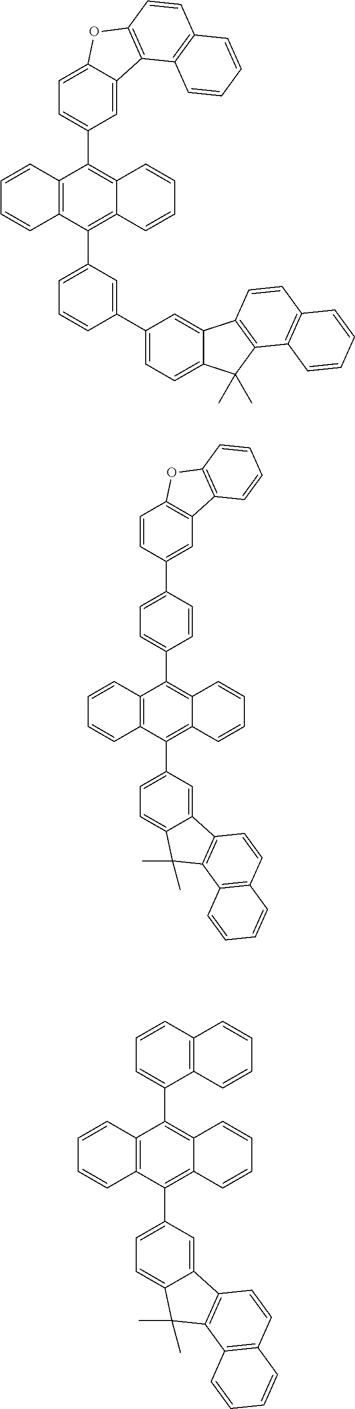

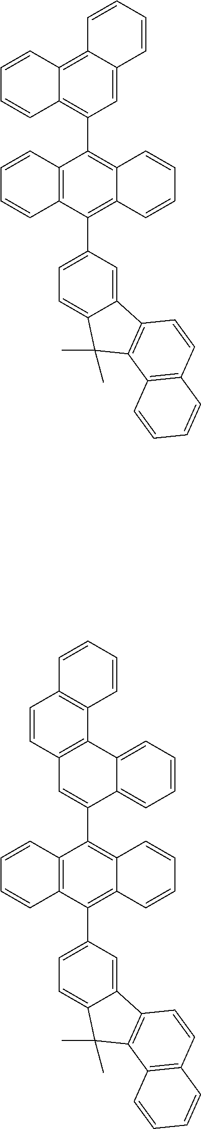

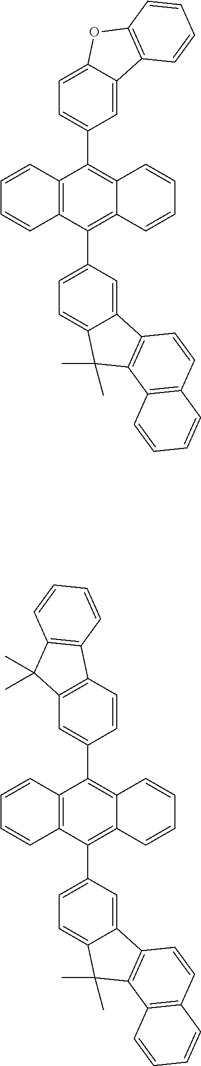

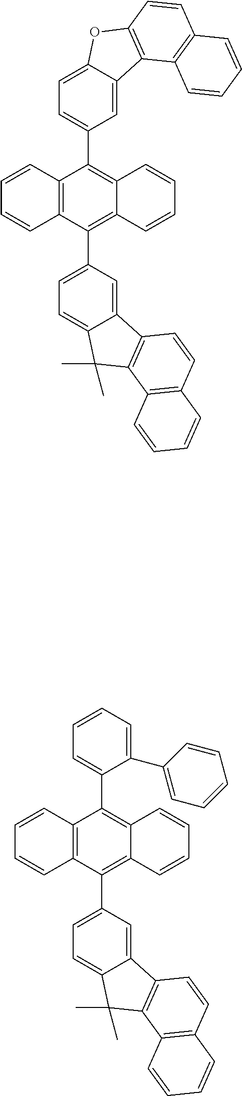

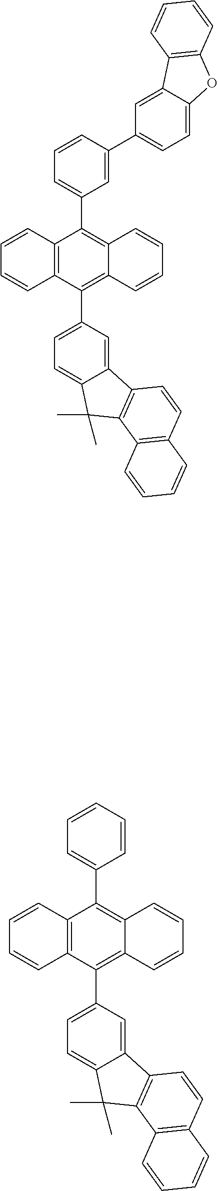

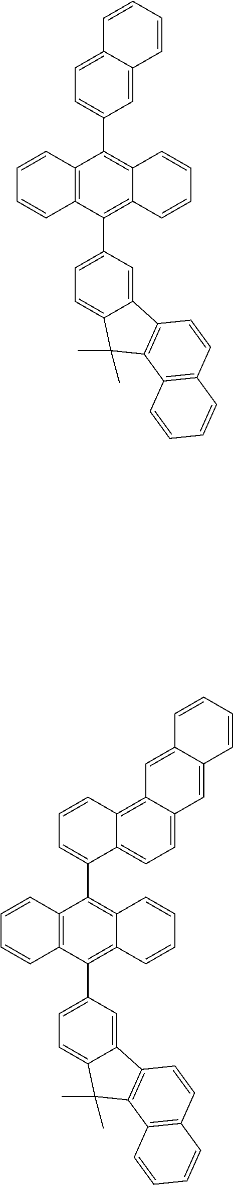

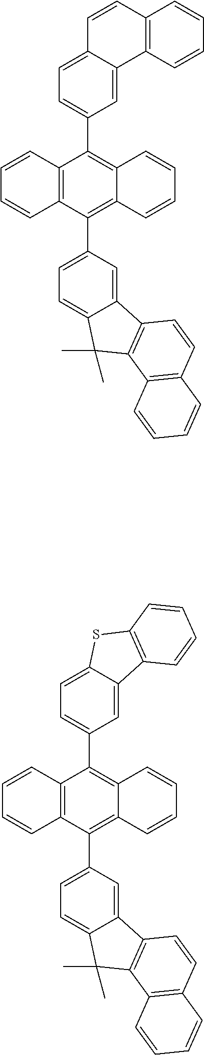

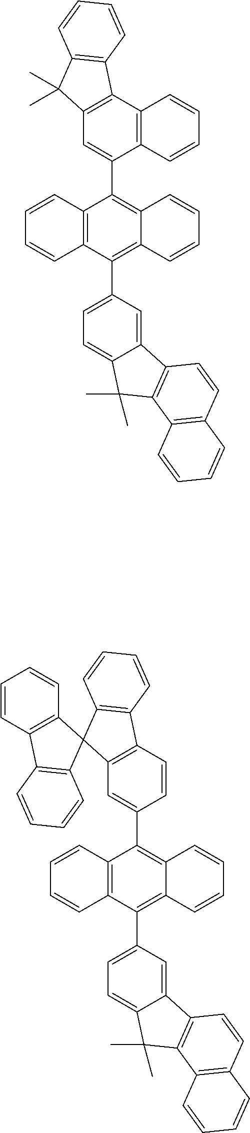

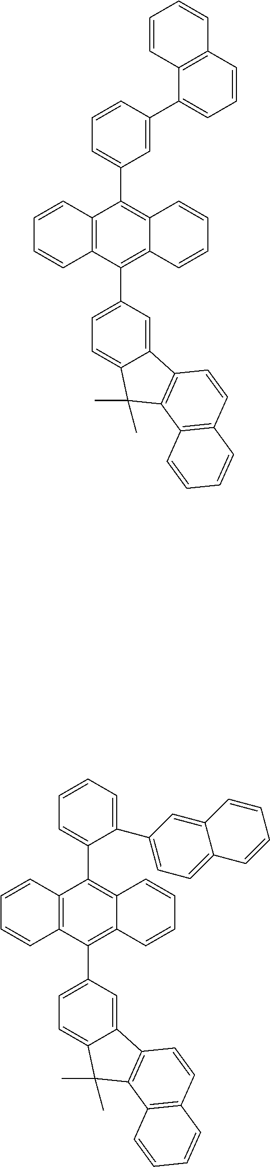

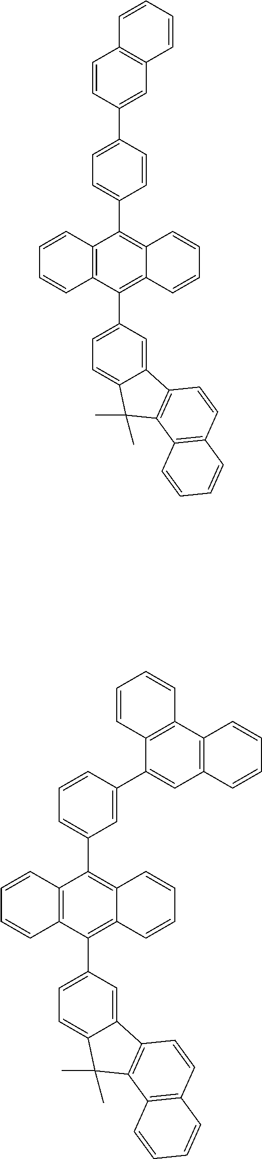

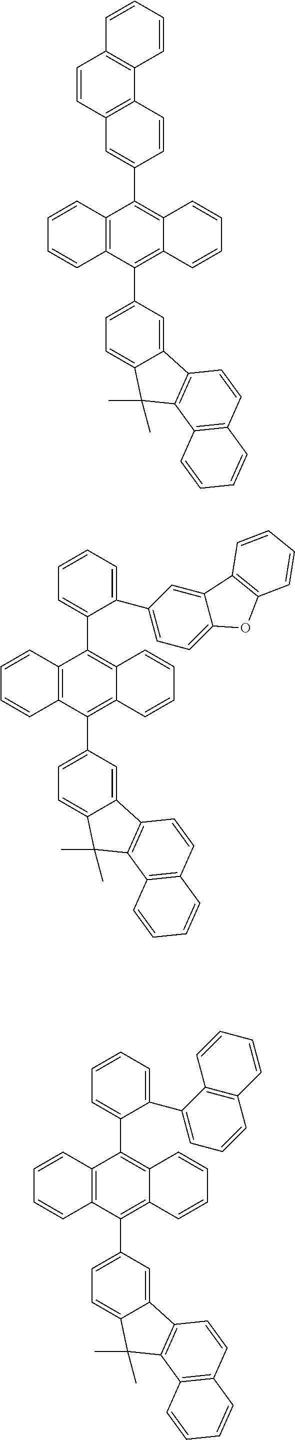

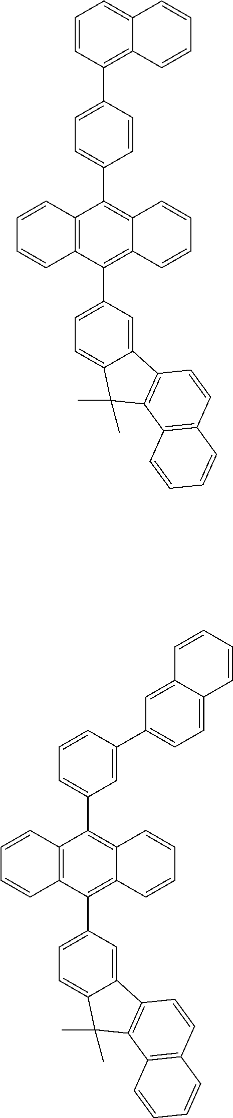

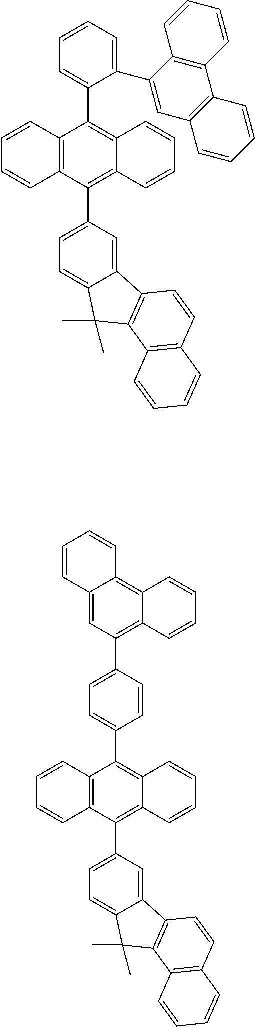

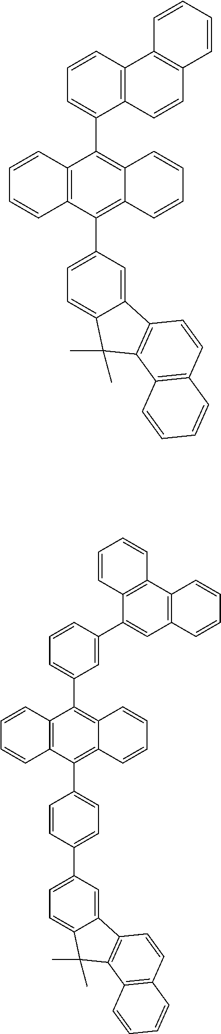

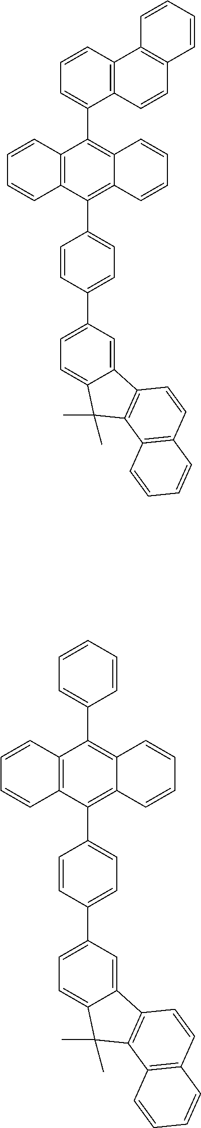

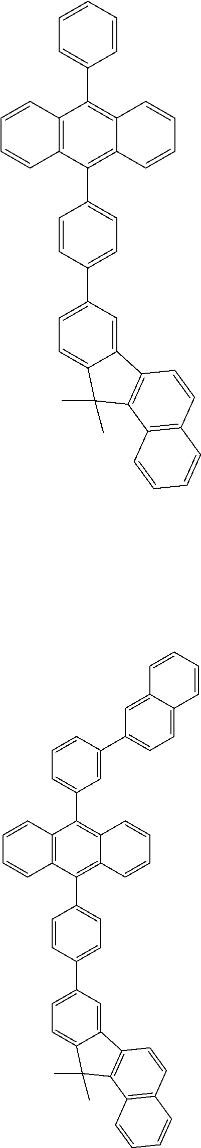

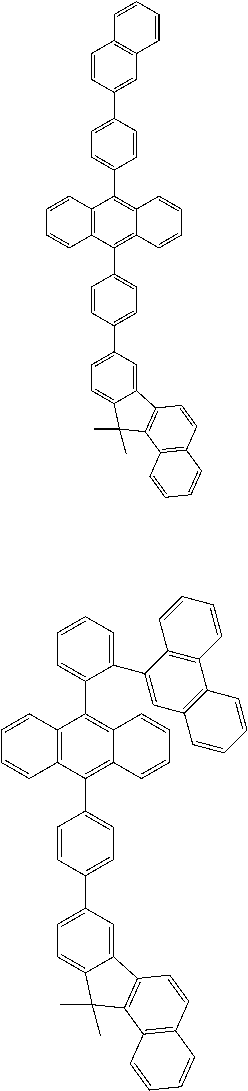

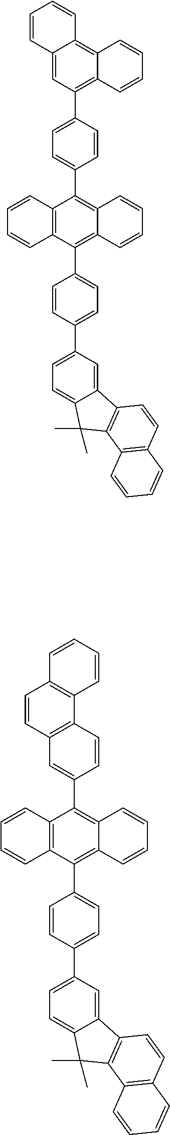

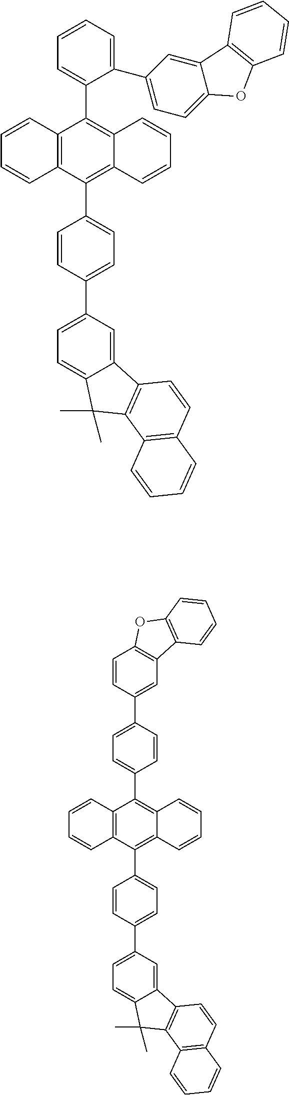 [SMILES: CC1(C)c2ccc(-c3ccc(-c4c5ccccc5c(-c5ccccc5-c5ccc6oc7ccccc7c6c5)c5ccccc45)cc3)cc2-c2ccc3ccccc3c21 ; CC1(C)c2ccc(-c3ccc(-c4c5ccccc5c(-c5ccc(-c6ccc7oc8ccccc8c7c6)cc5)c5ccccc45)cc3)cc2-c2ccc3ccccc3c21]

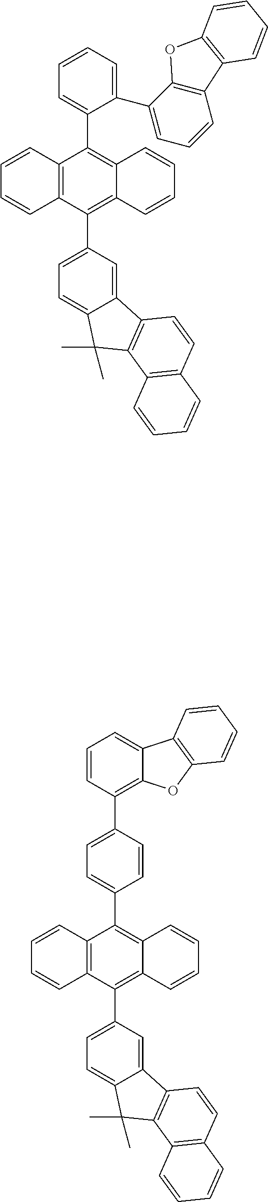 [SMILES: CC1(C)c2ccc(-c3c4ccccc4c(-c4ccccc4-c4cccc5c4oc4ccccc45)c4ccccc34)cc2-c2ccc3ccccc3c21 ; CC1(C)c2ccc(-c3c4ccccc4c(-c4ccc(-c5cccc6c5oc5ccccc56)cc4)c4ccccc34)cc2-c2ccc3ccccc3c21]

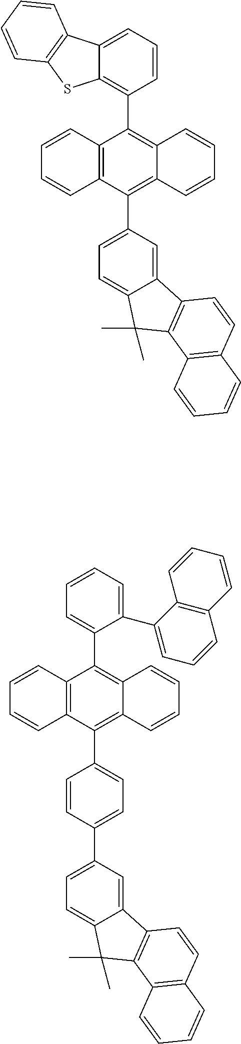

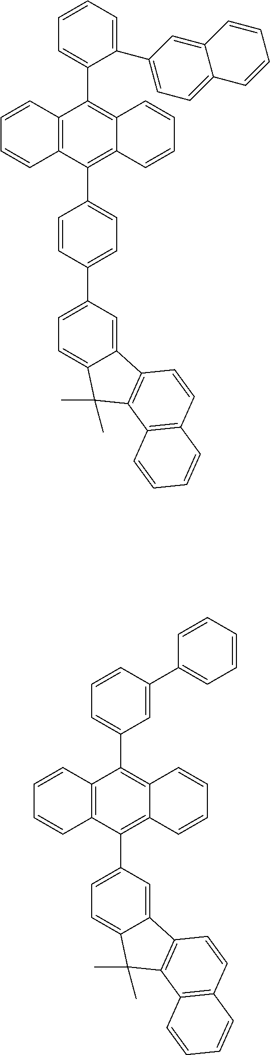

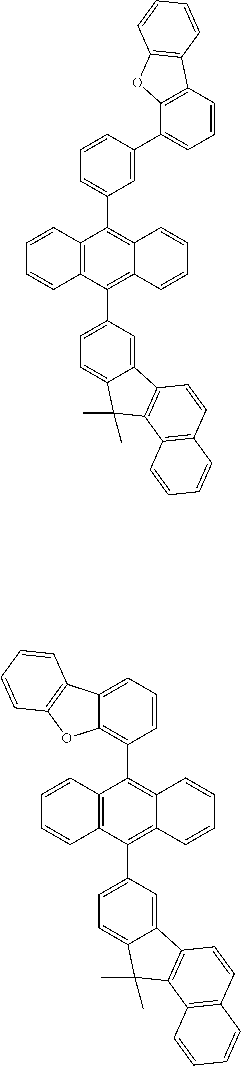 [SMILES: CC1(C)c2ccc(-c3c4ccccc4c(-c4cccc(-c5cccc6c5oc5ccccc56)c4)c4ccccc34)cc2-c2ccc3ccccc3c21 ; CC1(C)c2ccc(-c3c4ccccc4c(-c4cccc5c4oc4ccccc45)c4ccccc34)cc2-c2ccc3ccccc3c21]

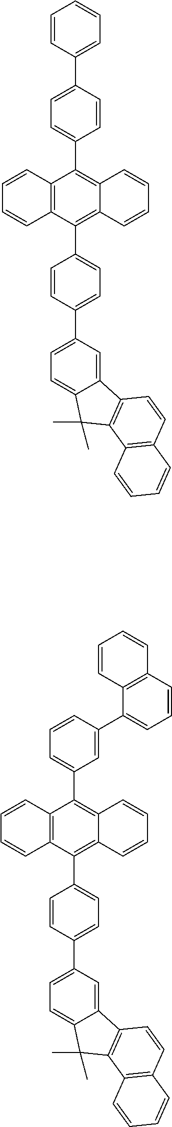

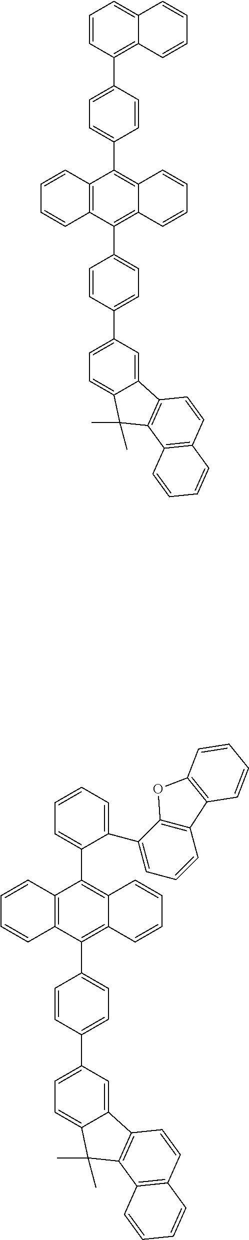

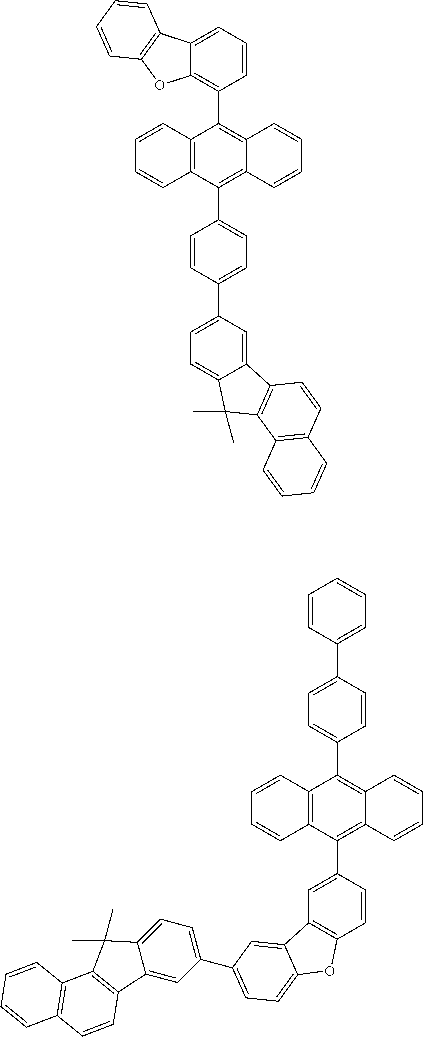 [SMILES: CC1(C)c2ccc(-c3ccc(-c4c5ccccc5c(-c5cccc6c5oc5ccccc56)c5ccccc45)cc3)cc2-c2ccc3ccccc3c21 ; CC1(C)c2ccc(-c3ccc4oc5ccc(-c6c7ccccc7c(-c7ccc(-c8ccccc8)cc7)c7ccccc67)cc5c4c3)cc2-c2ccc3ccccc3c21]

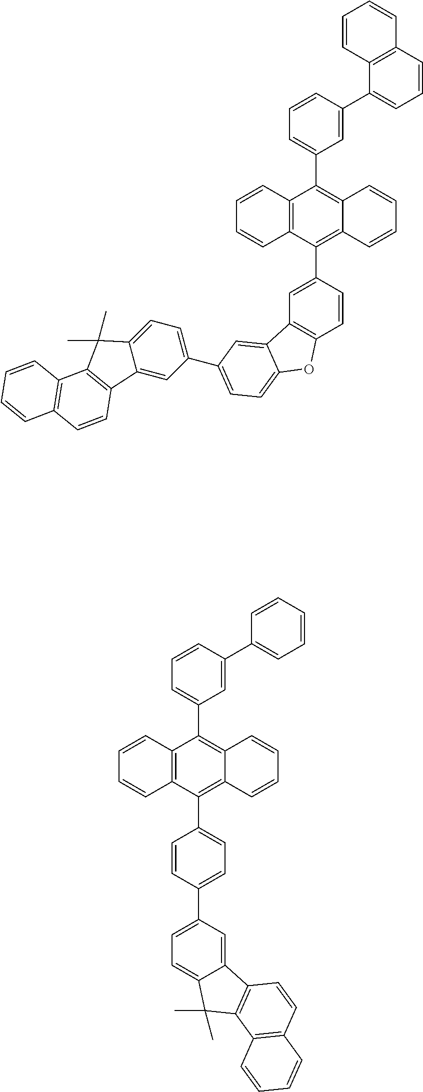

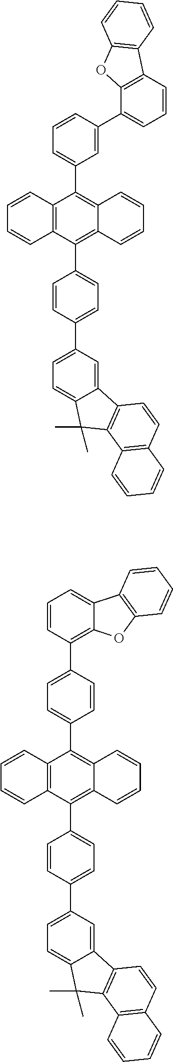 [SMILES: CC1(C)c2ccc(-c3ccc(-c4c5ccccc5c(-c5cccc(-c6cccc7c6oc6ccccc67)c5)c5ccccc45)cc3)cc2-c2ccc3ccccc3c21 ; CC1(C)c2ccc(-c3ccc(-c4c5ccccc5c(-c5ccc(-c6cccc7c6oc6ccccc67)cc5)c5ccccc45)cc3)cc2-c2ccc3ccccc3c21]

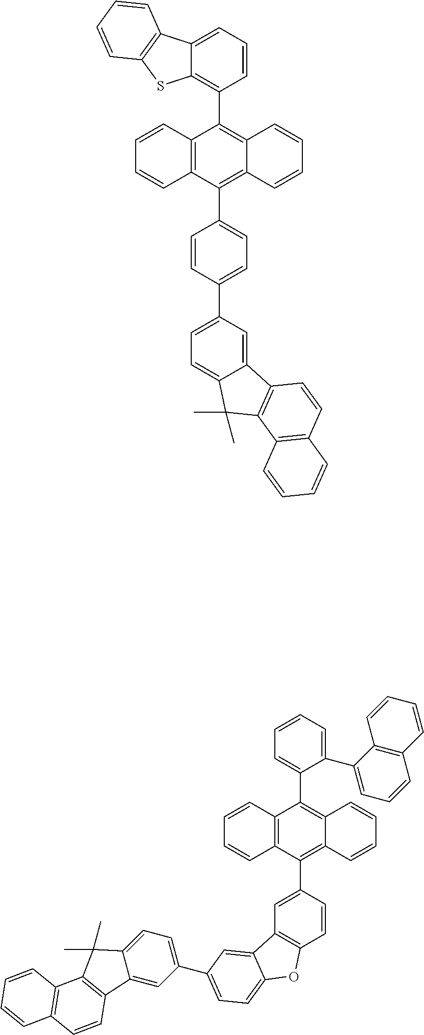 [SMILES: CC1(C)c2ccc(-c3ccc(-c4c5ccccc5c(-c5cccc6c5sc5ccccc56)c5ccccc45)cc3)cc2-c2ccc3ccccc3c21 ; CC1(C)c2ccc(-c3ccc4oc5ccc(-c6c7ccccc7c(-c7ccccc7-c7cccc8ccccc78)c7ccccc67)cc5c4c3)cc2-c2ccc3ccccc3c21]

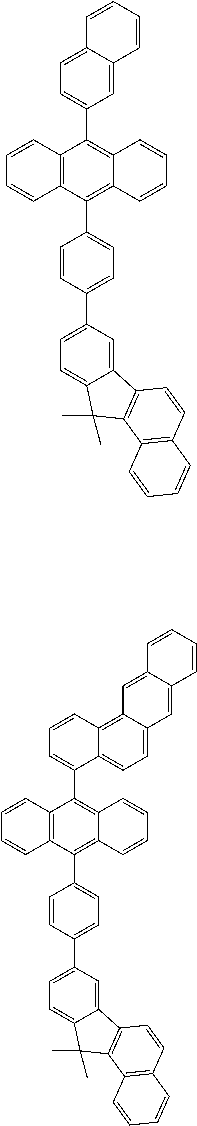

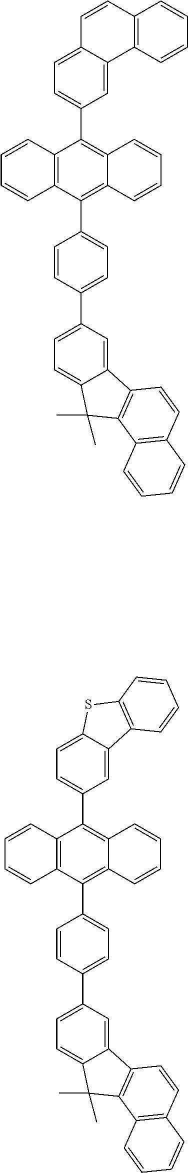

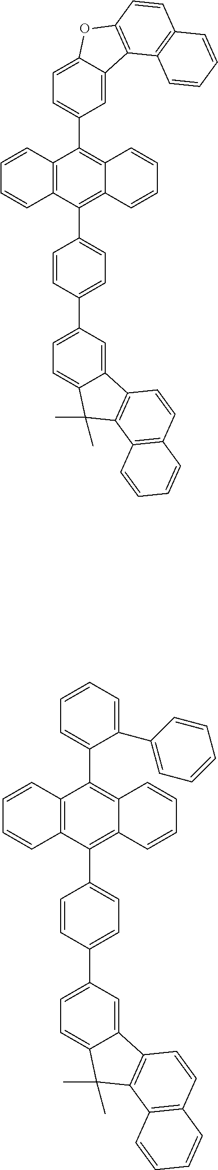

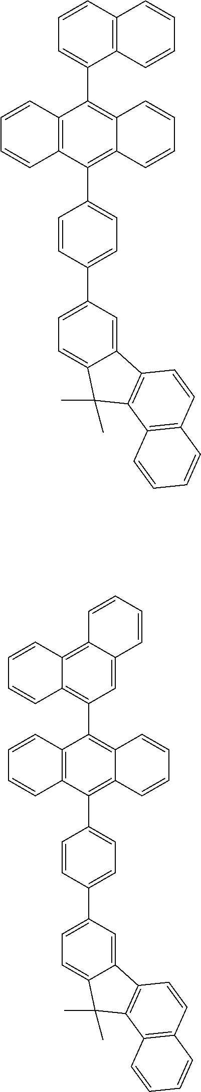

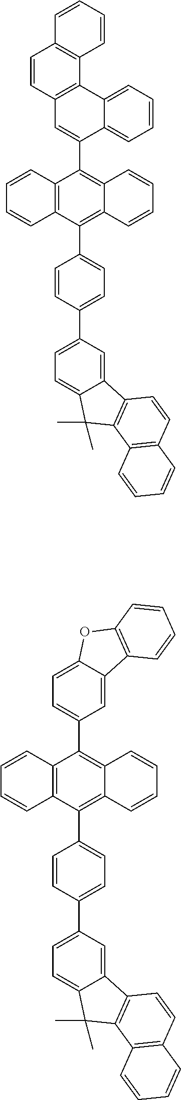

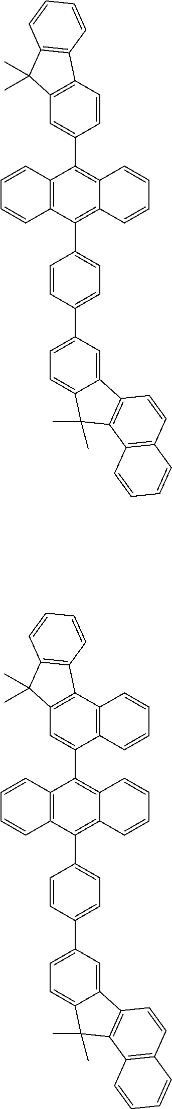

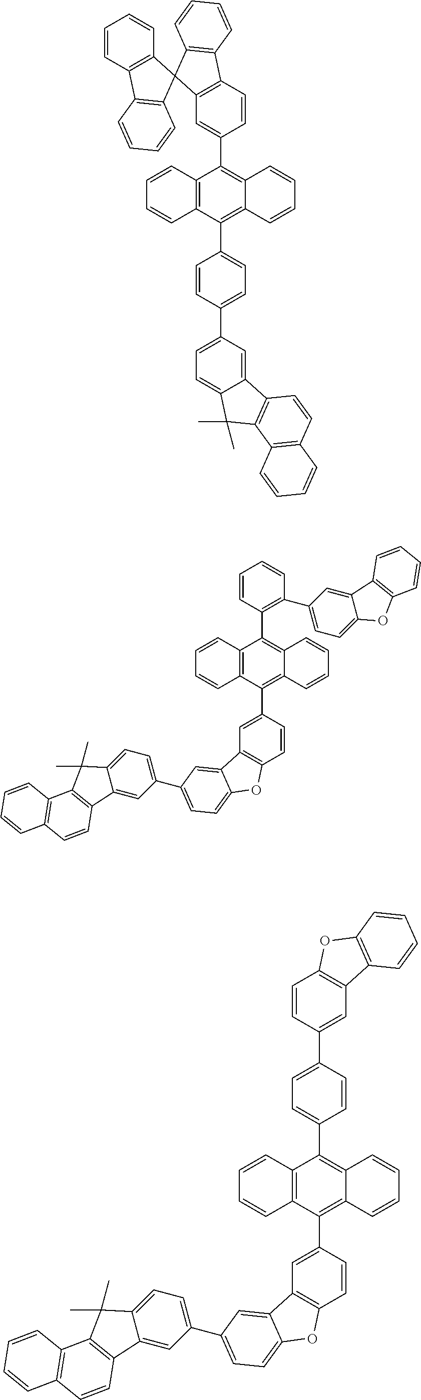

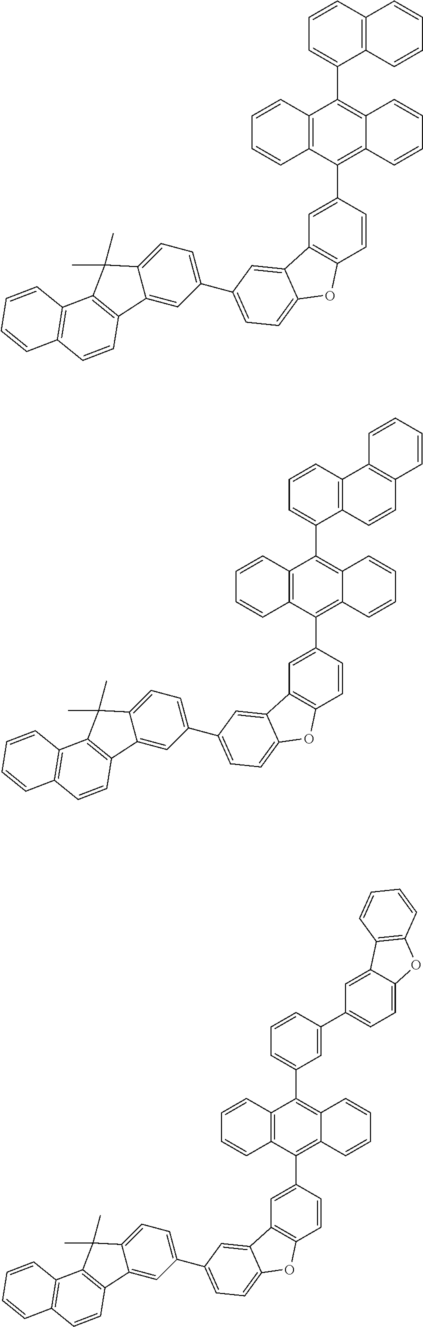 [SMILES: CC1(C)c2ccc(-c3ccc4oc5ccc(-c6c7ccccc7c(-c7cccc8ccccc78)c7ccccc67)cc5c4c3)cc2-c2ccc3ccccc3c21 ; CC1(C)c2ccc(-c3ccc4oc5ccc(-c6c7ccccc7c(-c7cccc8c7ccc7ccccc78)c7ccccc67)cc5c4c3)cc2-c2ccc3ccccc3c21 ; CC1(C)c2ccc(-c3ccc4oc5ccc(-c6c7ccccc7c(-c7cccc(-c8ccc9oc%10ccccc%10c9c8)c7)c7ccccc67)cc5c4c3)cc2-c2ccc3ccccc3c21]

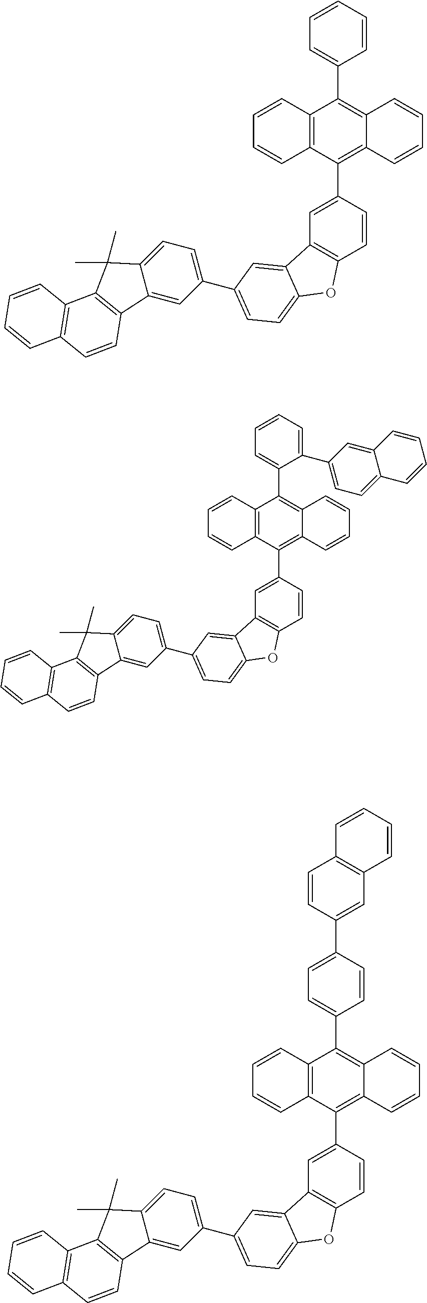 [SMILES: CC1(C)c2ccc(-c3ccc4oc5ccc(-c6c7ccccc7c(-c7ccccc7)c7ccccc67)cc5c4c3)cc2-c2ccc3ccccc3c21 ; CC1(C)c2ccc(-c3ccc4oc5ccc(-c6c7ccccc7c(-c7ccccc7-c7ccc8ccccc8c7)c7ccccc67)cc5c4c3)cc2-c2ccc3ccccc3c21 ; CC1(C)c2ccc(-c3ccc4oc5ccc(-c6c7ccccc7c(-c7ccc(-c8ccc9ccccc9c8)cc7)c7ccccc67)cc5c4c3)cc2-c2ccc3ccccc3c21]

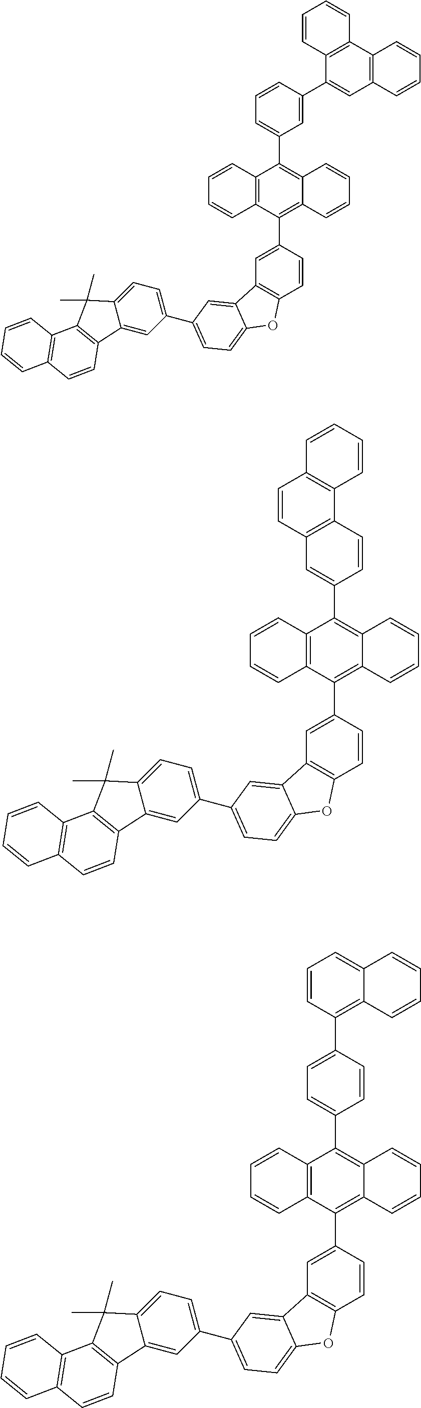 [SMILES: CC1(C)c2ccc(-c3ccc4oc5ccc(-c6c7ccccc7c(-c7cccc(-c8cc9ccccc9c9ccccc89)c7)c7ccccc67)cc5c4c3)cc2-c2ccc3ccccc3c21 ; CC1(C)c2ccc(-c3ccc4oc5ccc(-c6c7ccccc7c(-c7ccc8c(ccc9ccccc98)c7)c7ccccc67)cc5c4c3)cc2-c2ccc3ccccc3c21 ; CC1(C)c2ccc(-c3ccc4oc5ccc(-c6c7ccccc7c(-c7ccc(-c8cccc9ccccc89)cc7)c7ccccc67)cc5c4c3)cc2-c2ccc3ccccc3c21]

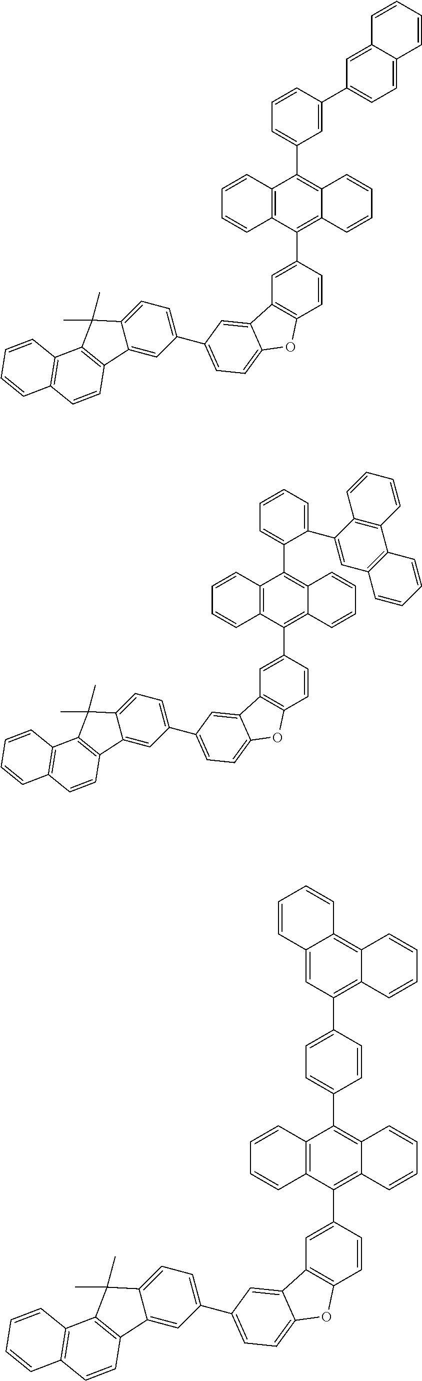 [SMILES: CC1(C)c2ccc(-c3ccc4oc5ccc(-c6c7ccccc7c(-c7cccc(-c8ccc9ccccc9c8)c7)c7ccccc67)cc5c4c3)cc2-c2ccc3ccccc3c21 ; CC1(C)c2ccc(-c3ccc4oc5ccc(-c6c7ccccc7c(-c7ccccc7-c7cc8ccccc8c8ccccc78)c7ccccc67)cc5c4c3)cc2-c2ccc3ccccc3c21 ; CC1(C)c2ccc(-c3ccc4oc5ccc(-c6c7ccccc7c(-c7ccc(-c8cc9ccccc9c9ccccc89)cc7)c7ccccc67)cc5c4c3)cc2-c2ccc3ccccc3c21]

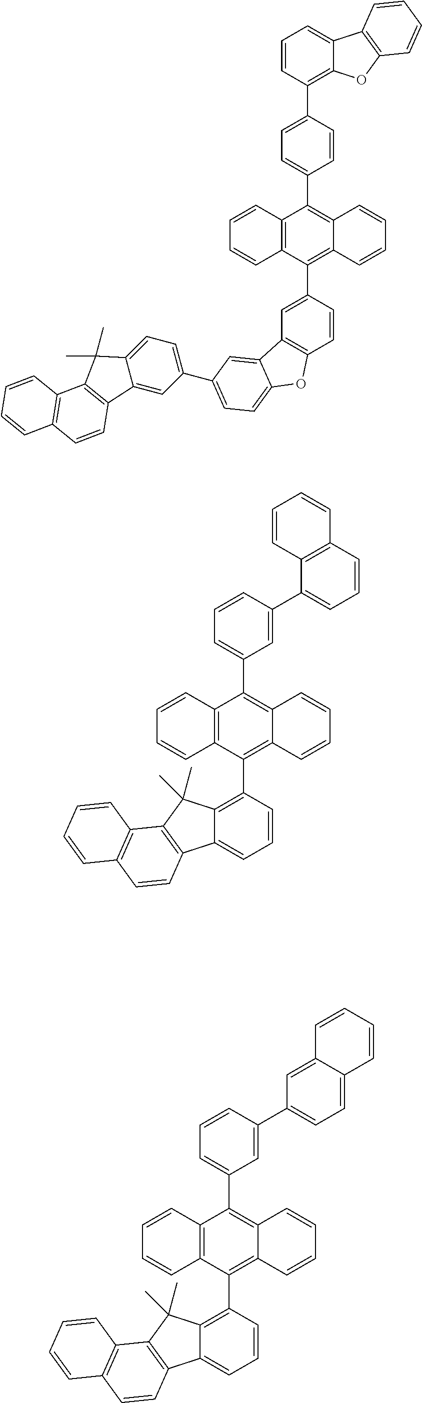

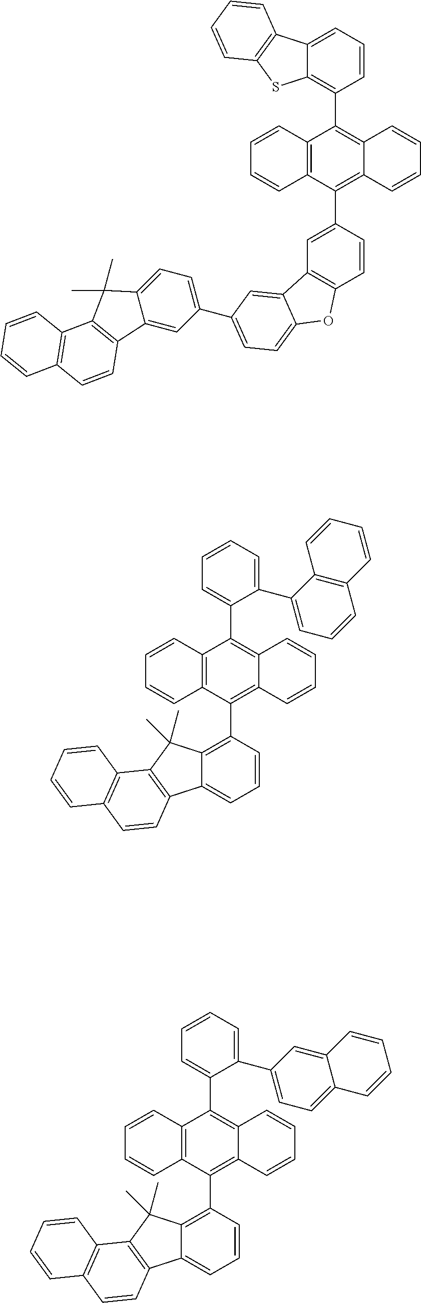

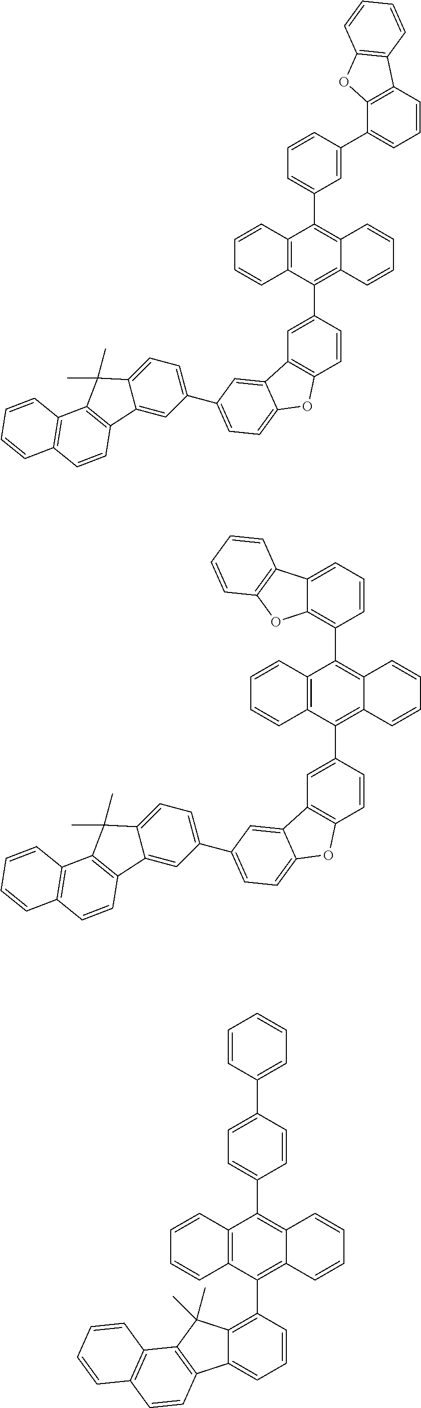

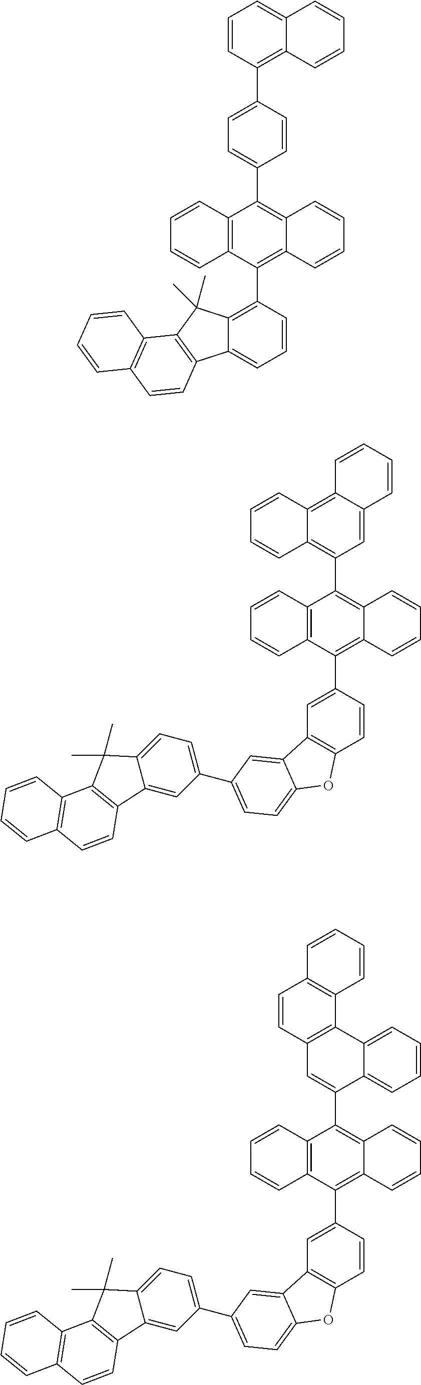

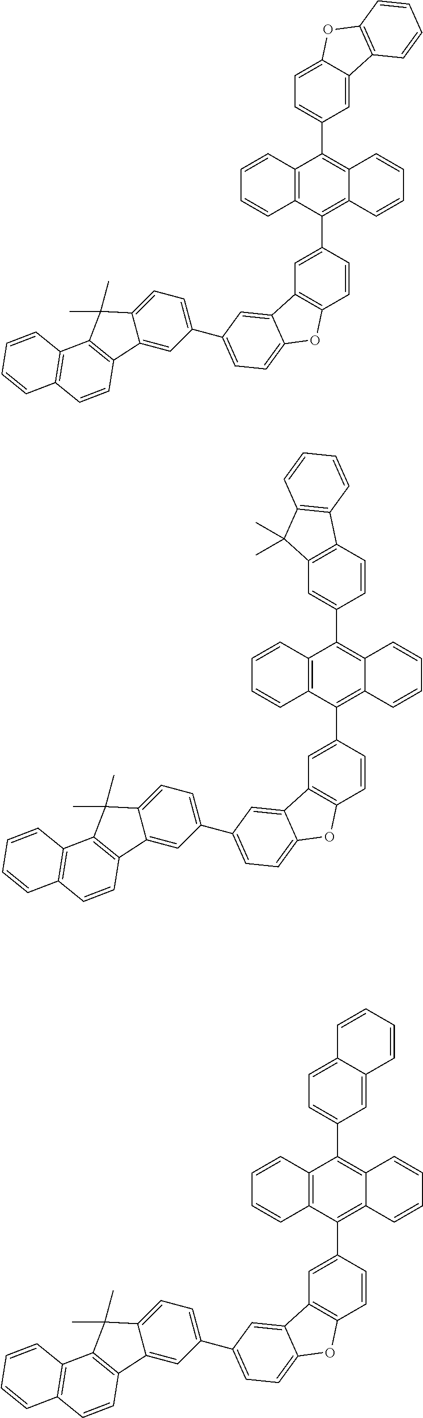 [SMILES: CC1(C)c2ccc(-c3ccc4oc5ccc(-c6c7ccccc7c(-c7ccc8oc9ccccc9c8c7)c7ccccc67)cc5c4c3)cc2-c2ccc3ccccc3c21 ; CC1(C)c2ccccc2-c2ccc(-c3c4ccccc4c(-c4ccc5oc6ccc(-c7ccc8c(c7)-c7ccc9ccccc9c7C8(C)C)cc6c5c4)c4ccccc34)cc21 ; CC1(C)c2ccc(-c3ccc4oc5ccc(-c6c7ccccc7c(-c7ccc8ccccc8c7)c7ccccc67)cc5c4c3)cc2-c2ccc3ccccc3c21]

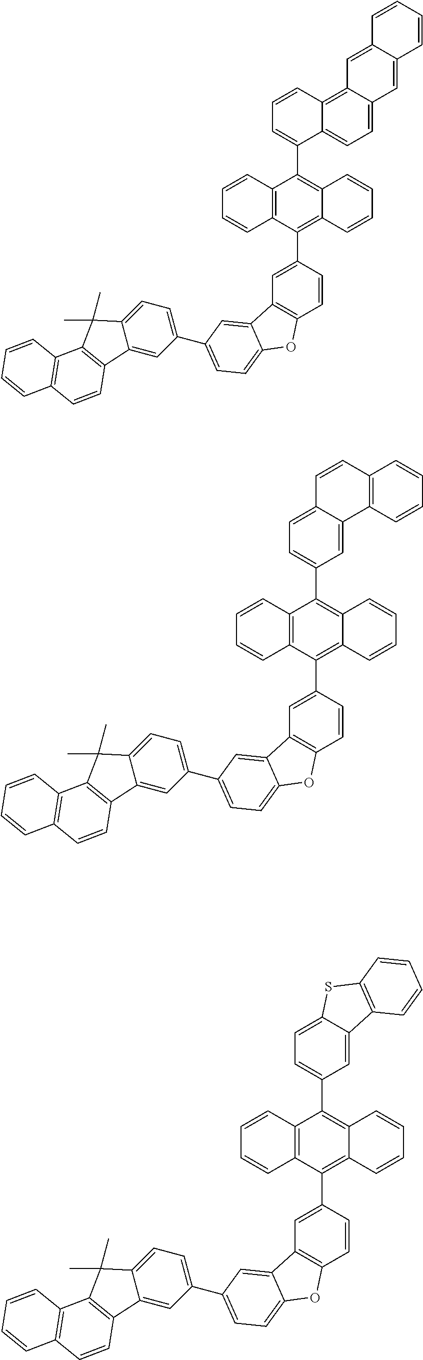 [SMILES: CC1(C)c2ccc(-c3ccc4oc5ccc(-c6c7ccccc7c(-c7cccc8c7ccc7cc9ccccc9cc78)c7ccccc67)cc5c4c3)cc2-c2ccc3ccccc3c21 ; CC1(C)c2ccc(-c3ccc4oc5ccc(-c6c7ccccc7c(-c7ccc8ccc9ccccc9c8c7)c7ccccc67)cc5c4c3)cc2-c2ccc3ccccc3c21 ; CC1(C)c2ccc(-c3ccc4oc5ccc(-c6c7ccccc7c(-c7ccc8sc9ccccc9c8c7)c7ccccc67)cc5c4c3)cc2-c2ccc3ccccc3c21]

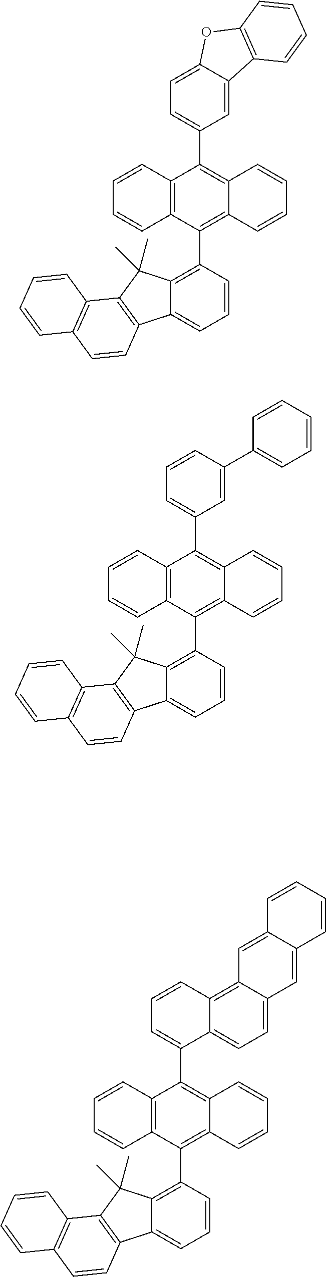

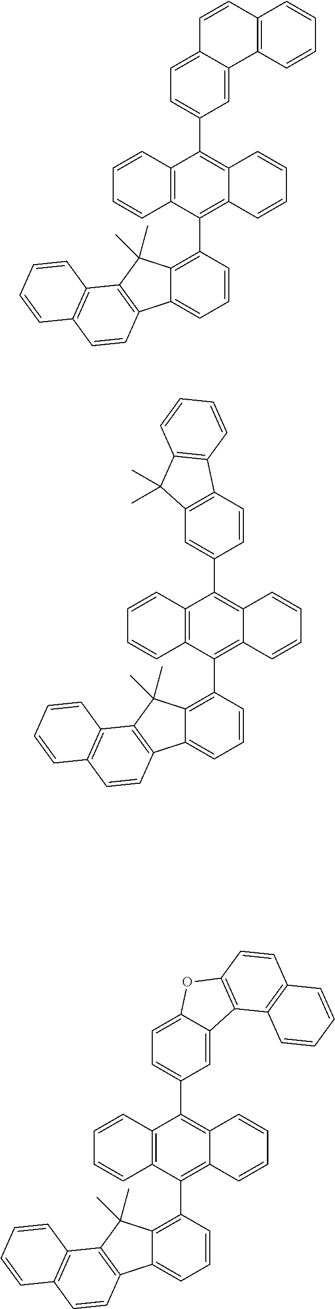

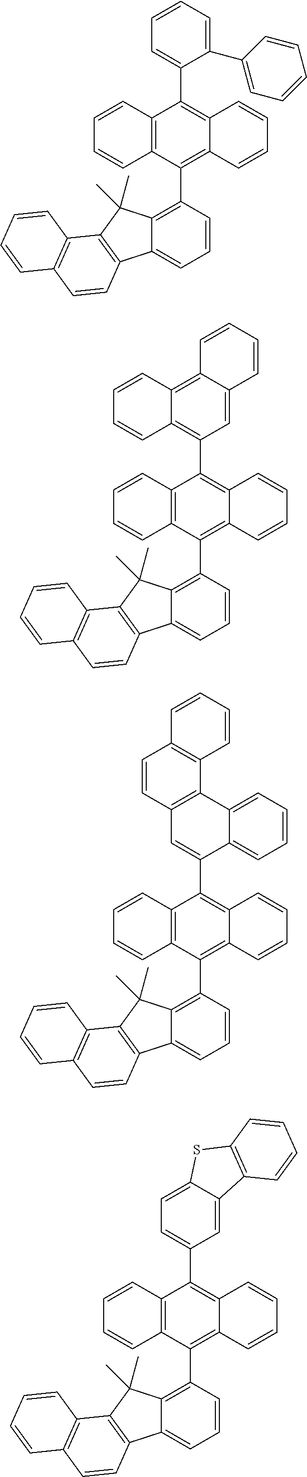

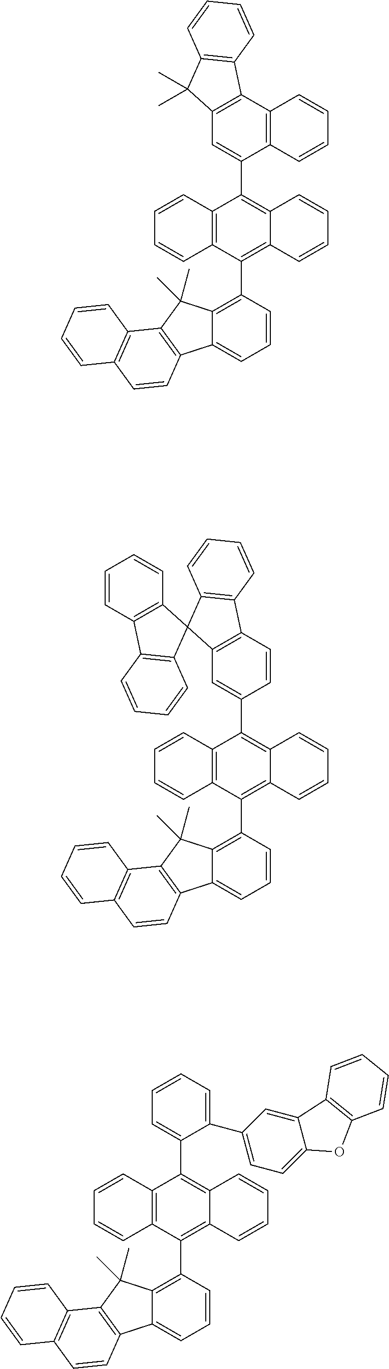

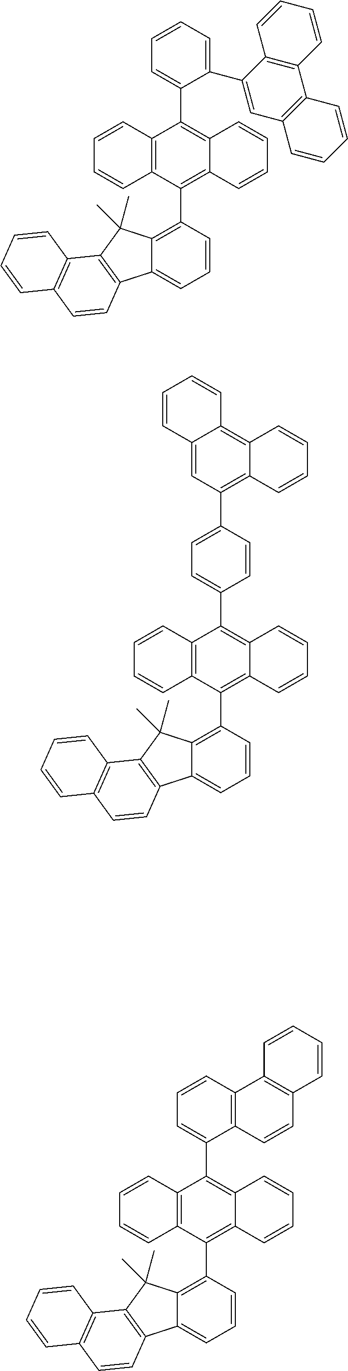

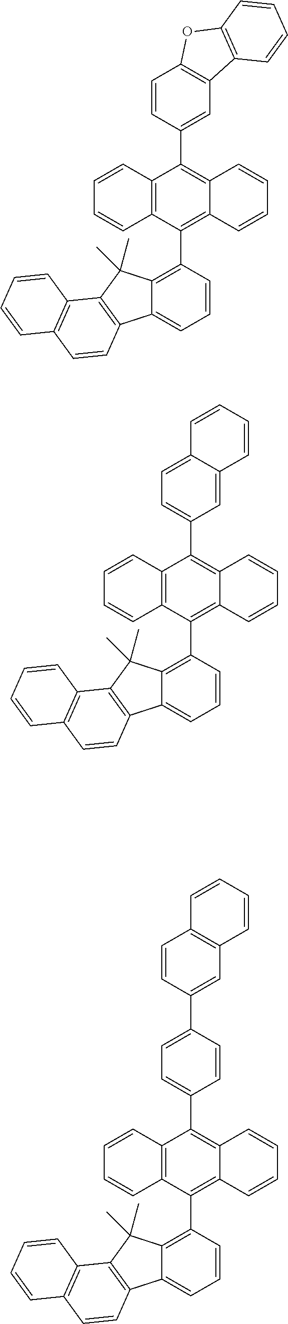

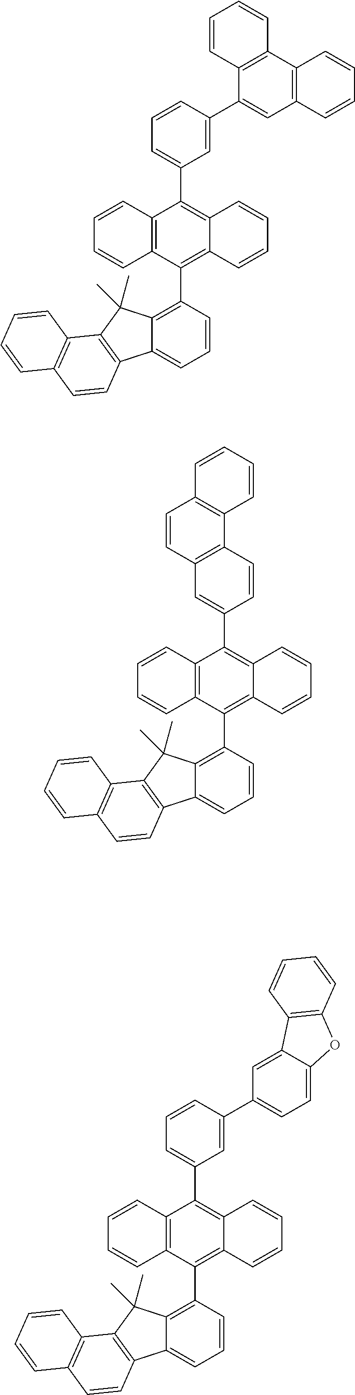

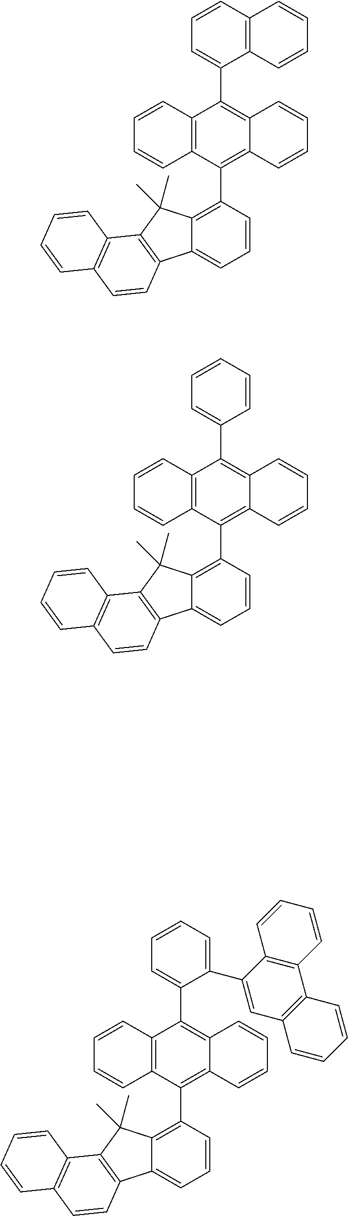

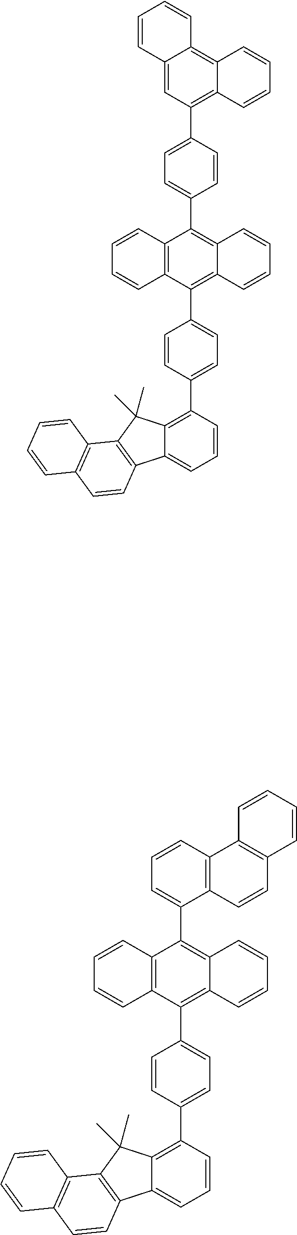

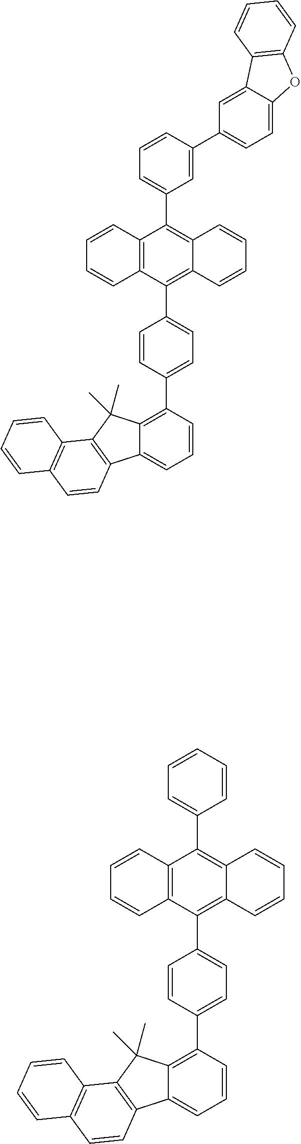

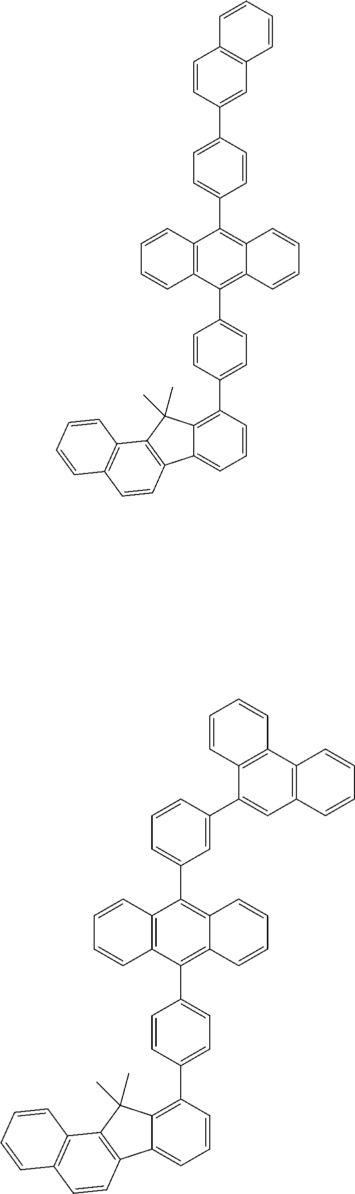

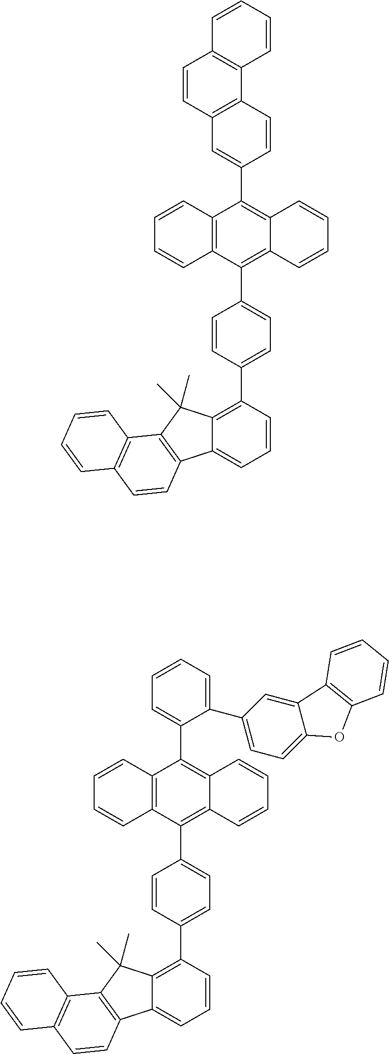

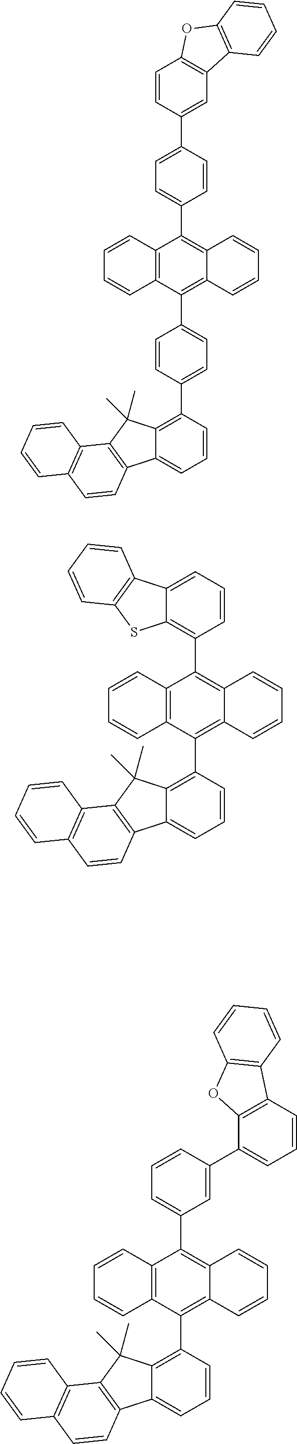

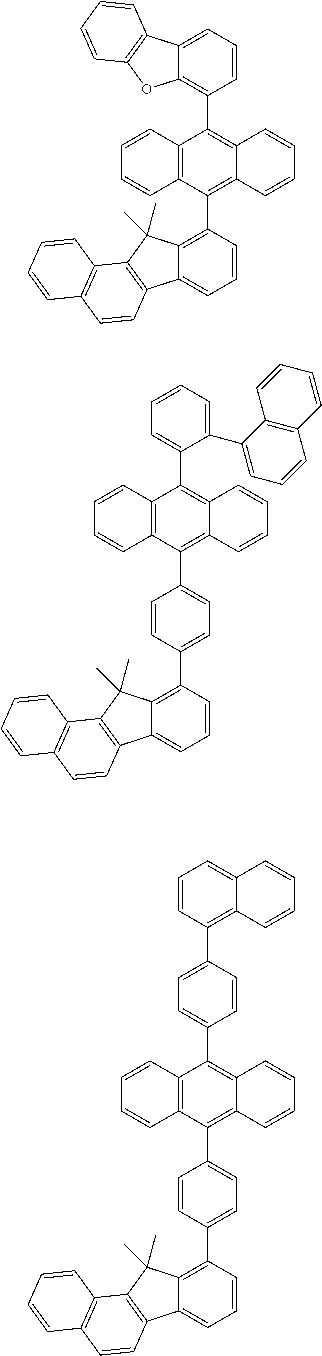

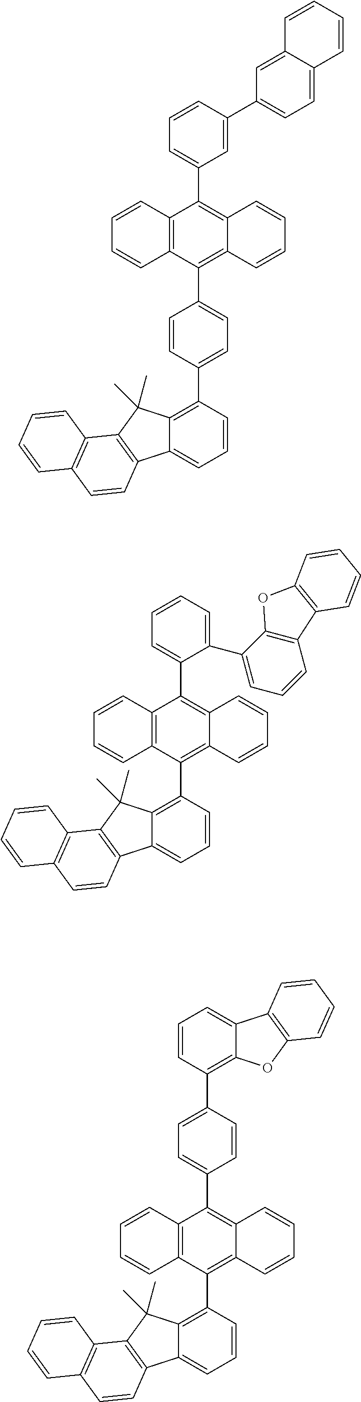

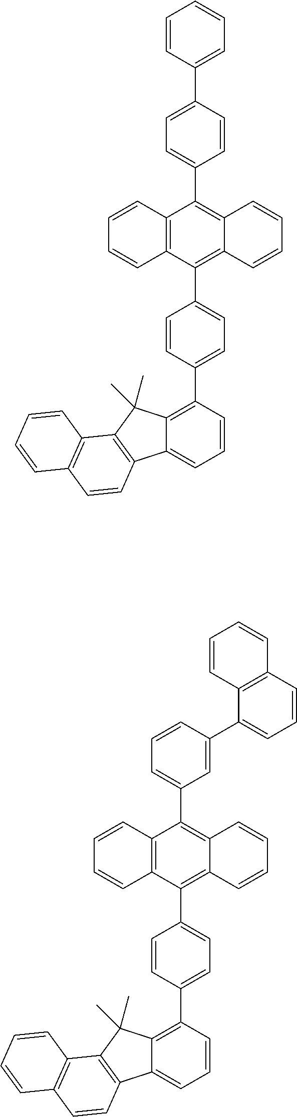

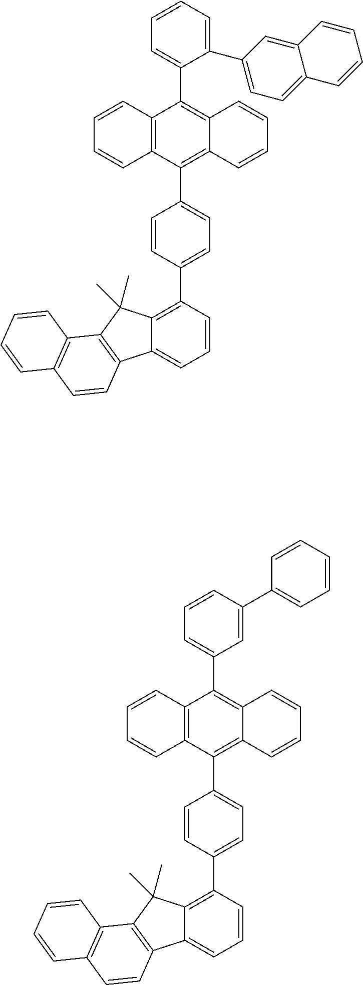

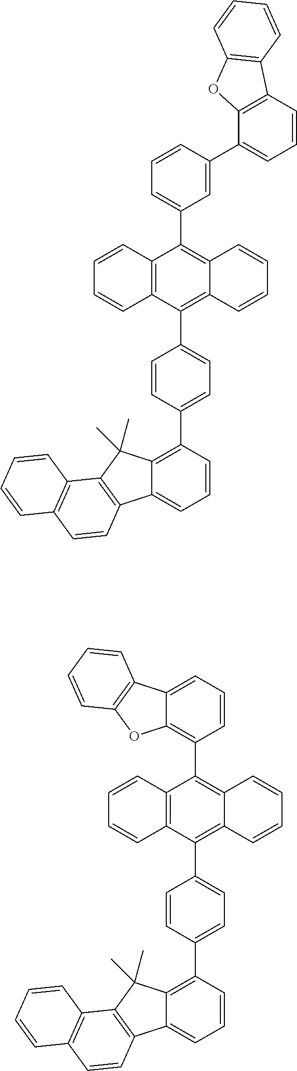 [SMILES: CC1(C)c2c(-c3ccc(-c4c5ccccc5c(-c5cccc(-c6cccc7c6oc6ccccc67)c5)c5ccccc45)cc3)cccc2-c2ccc3ccccc3c21 ; CC1(C)c2c(-c3ccc(-c4c5ccccc5c(-c5cccc6c5oc5ccccc56)c5ccccc45)cc3)cccc2-c2ccc3ccccc3c21]

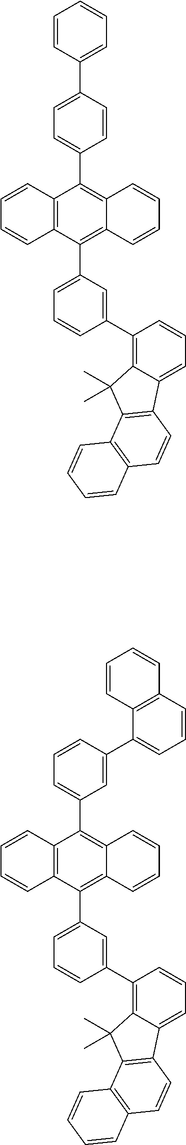

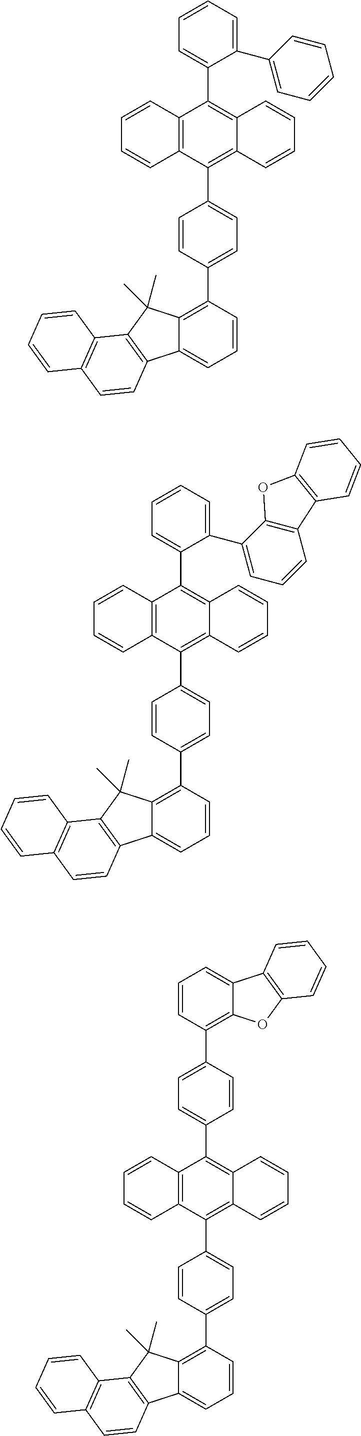

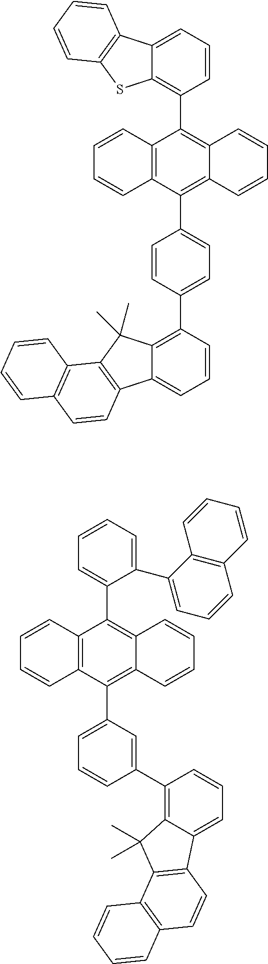

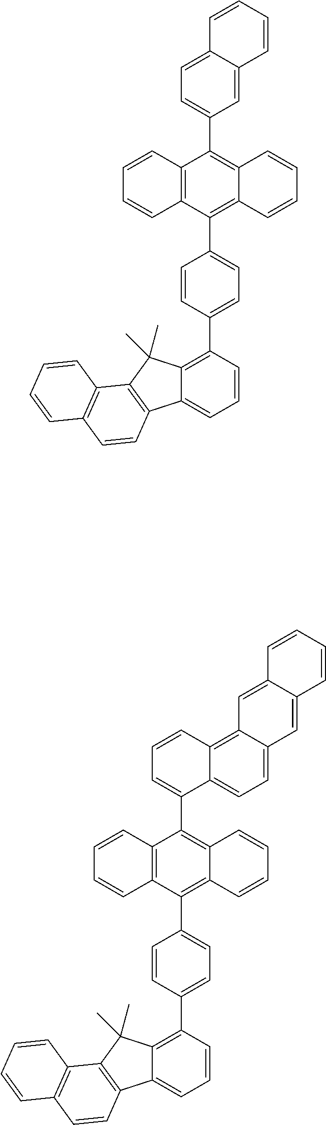

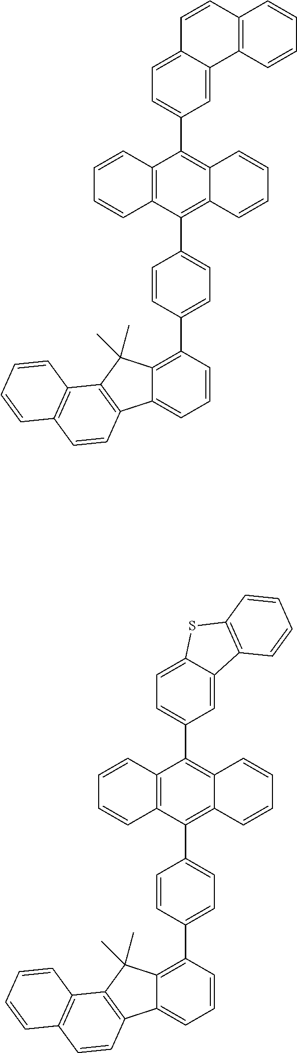

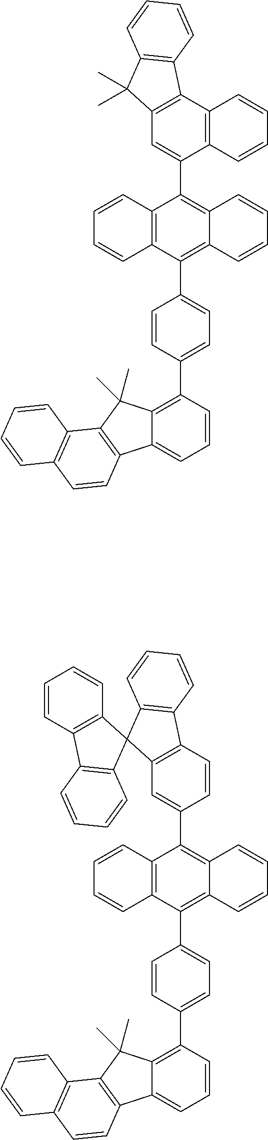

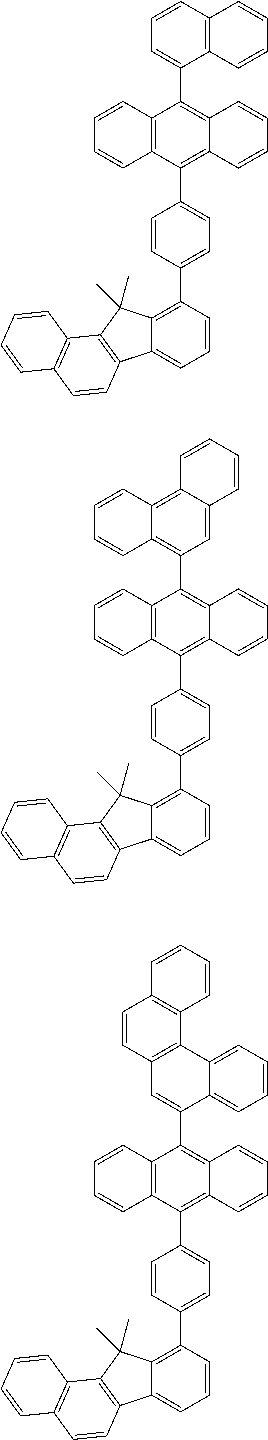

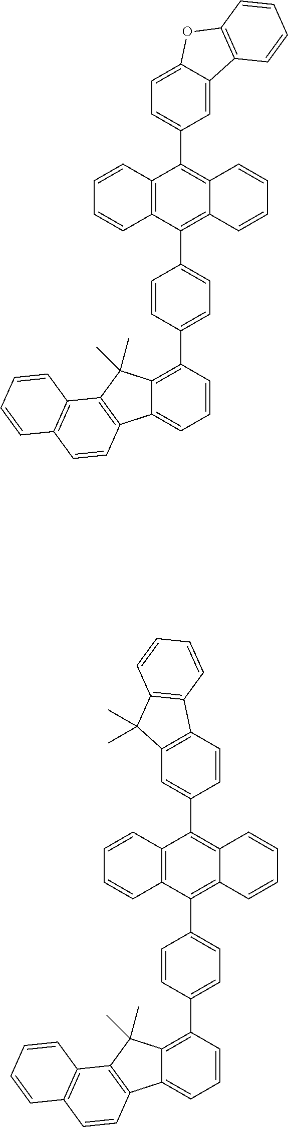

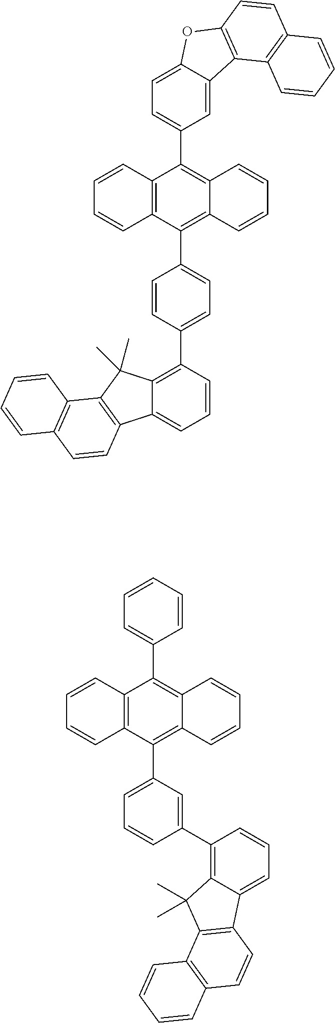

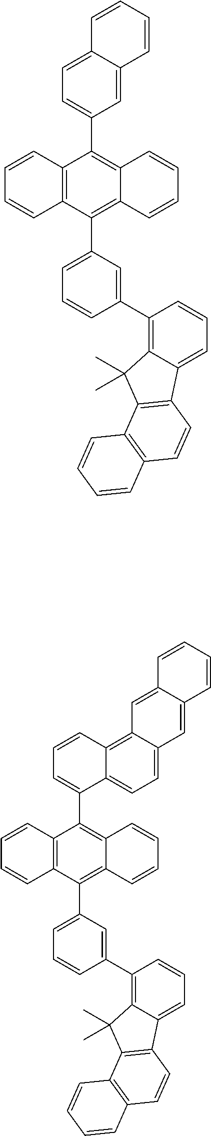

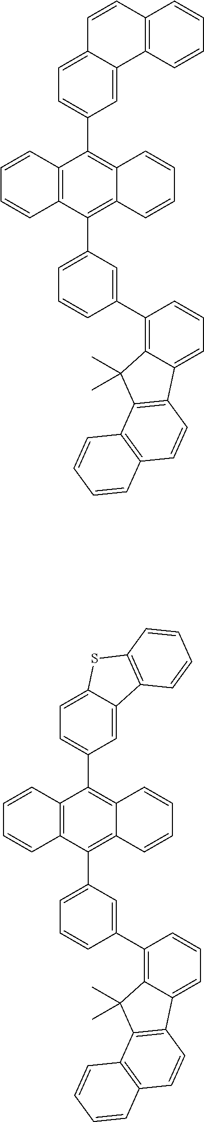

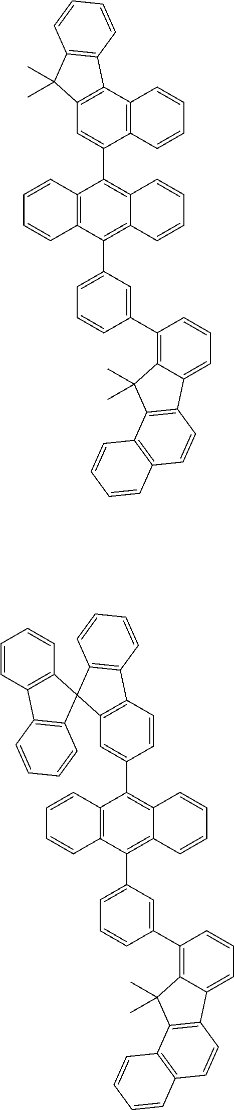

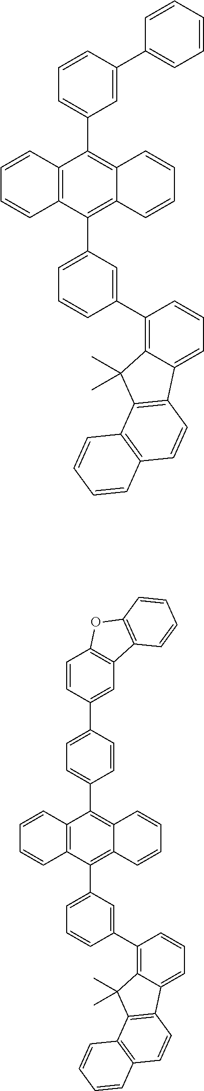

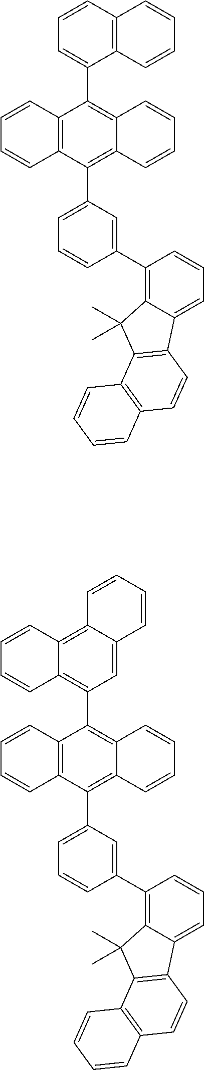

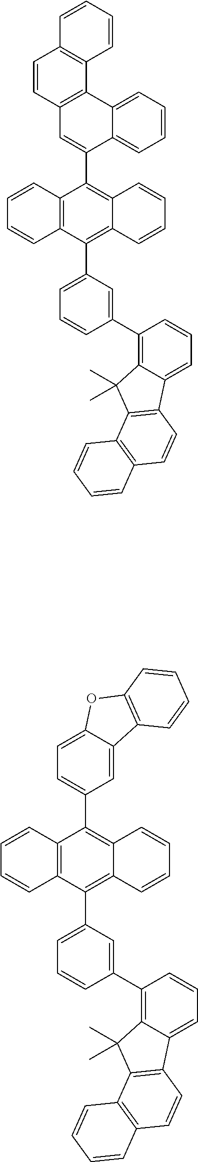

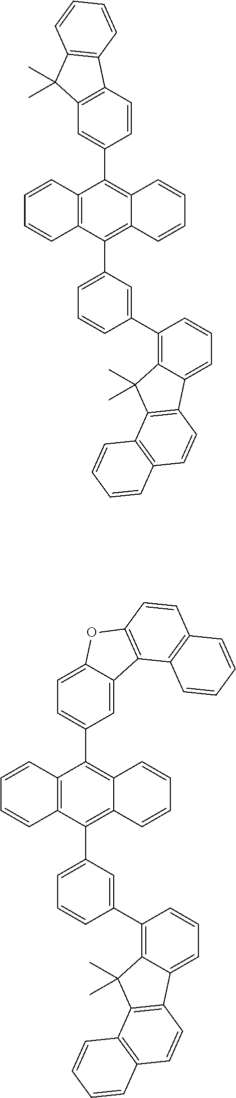

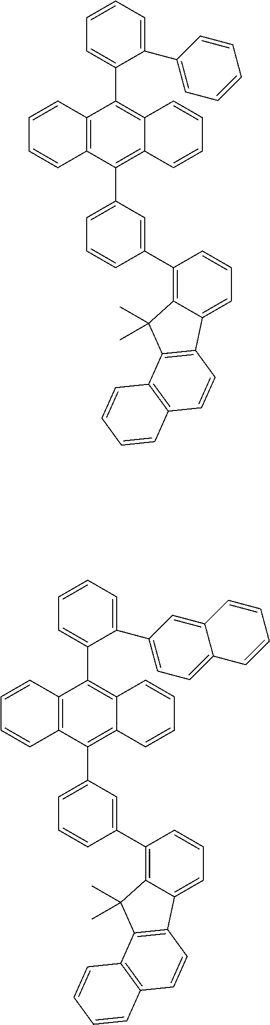

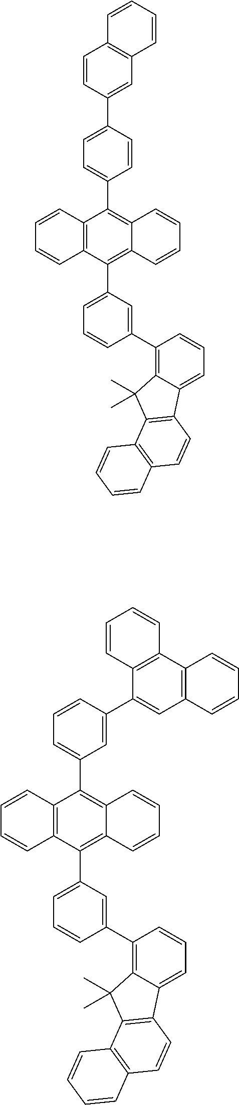

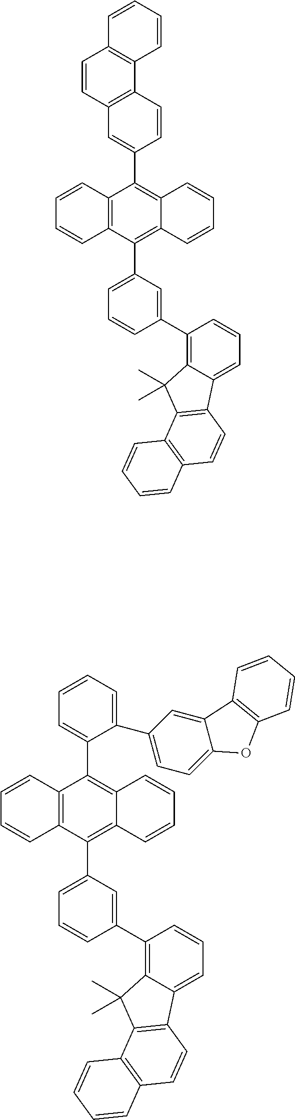

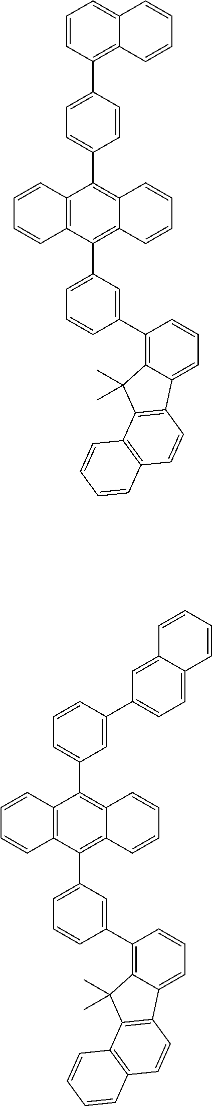

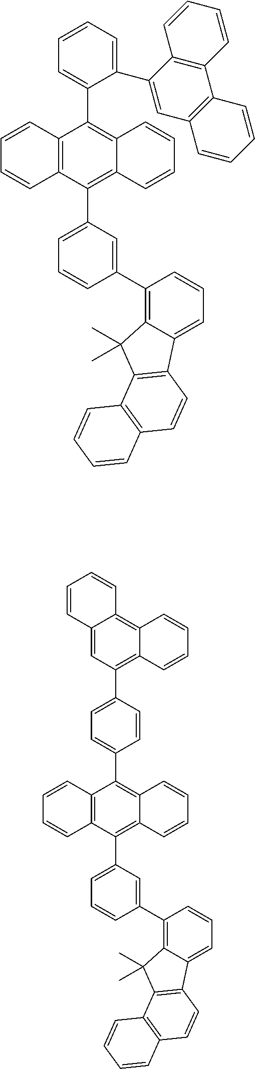

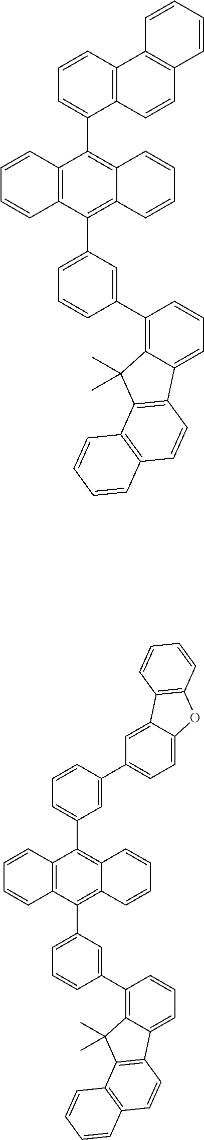

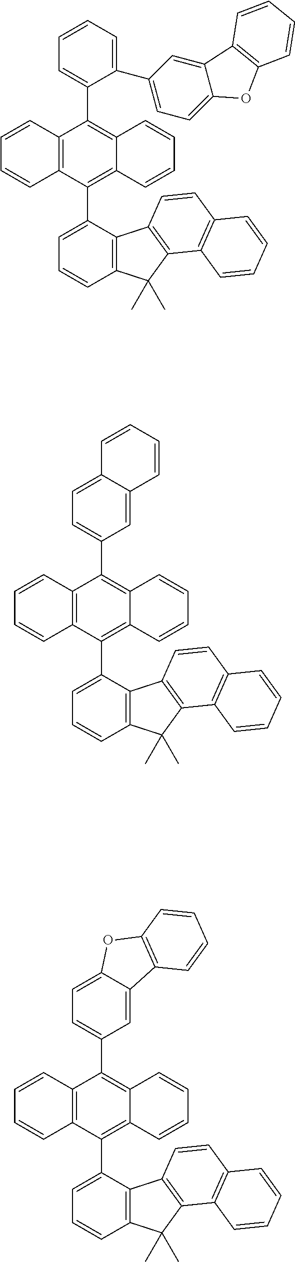

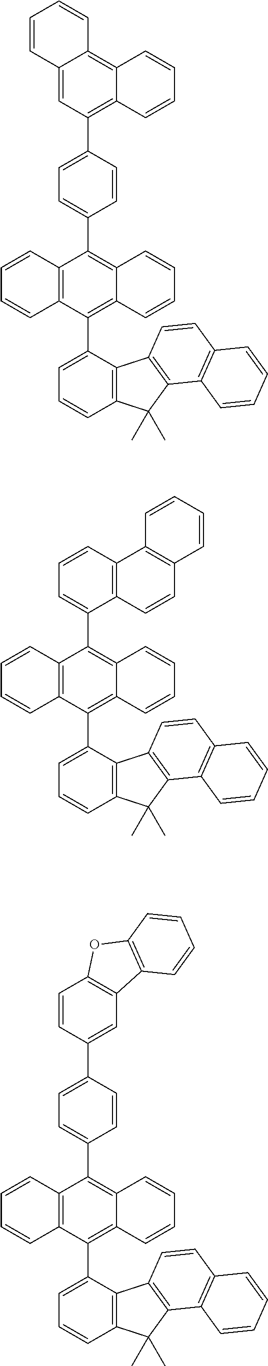

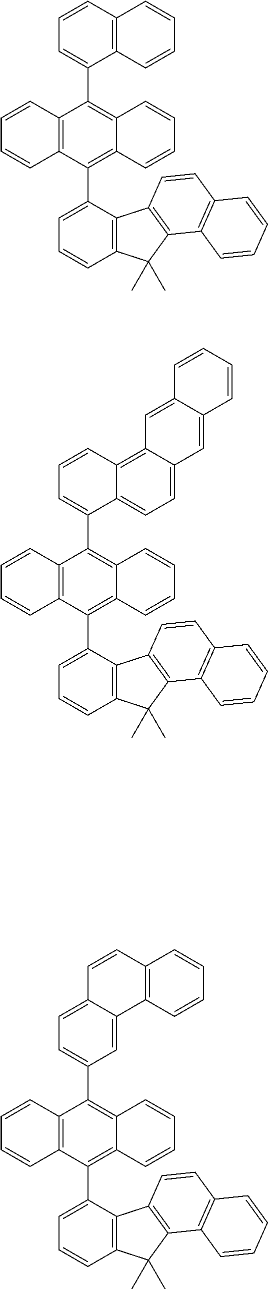

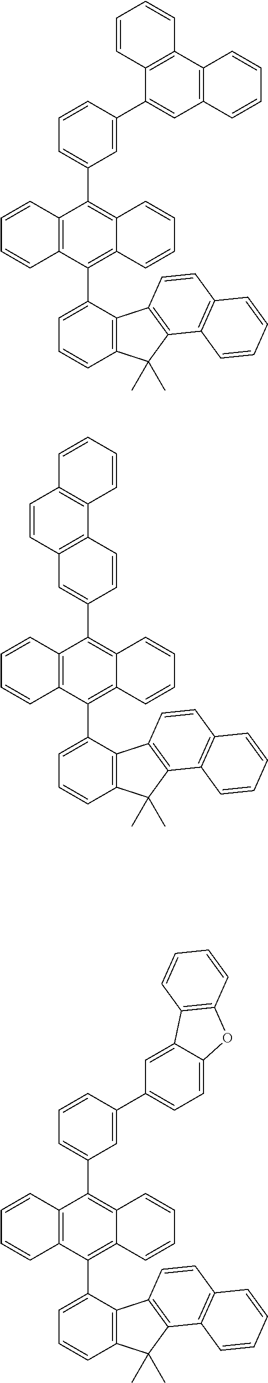

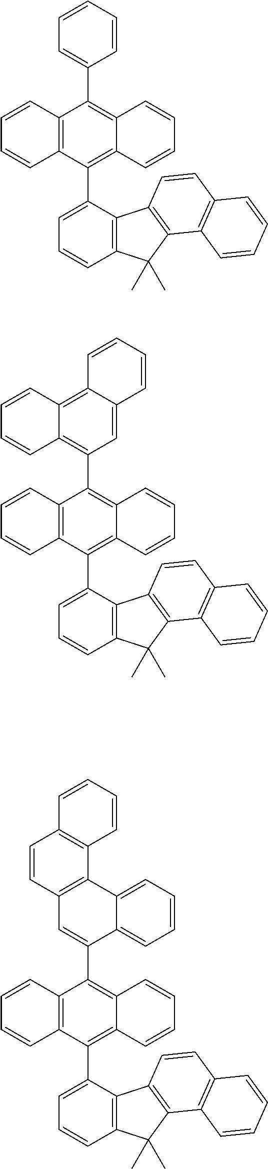

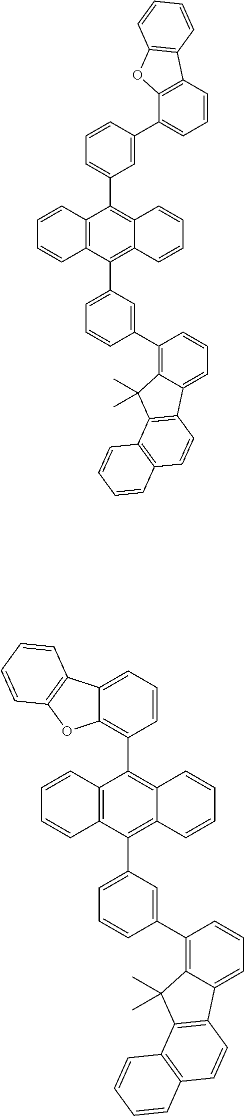 [SMILES: CC1(C)c2c(-c3cccc(-c4c5ccccc5c(-c5cccc(-c6cccc7c6oc6ccccc67)c5)c5ccccc45)c3)cccc2-c2ccc3ccccc3c21 ; CC1(C)c2c(-c3cccc(-c4c5ccccc5c(-c5cccc6c5oc5ccccc56)c5ccccc45)c3)cccc2-c2ccc3ccccc3c21]

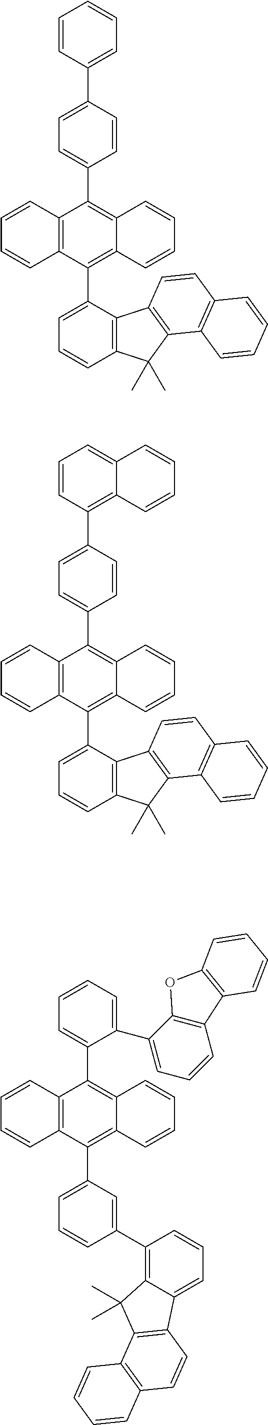

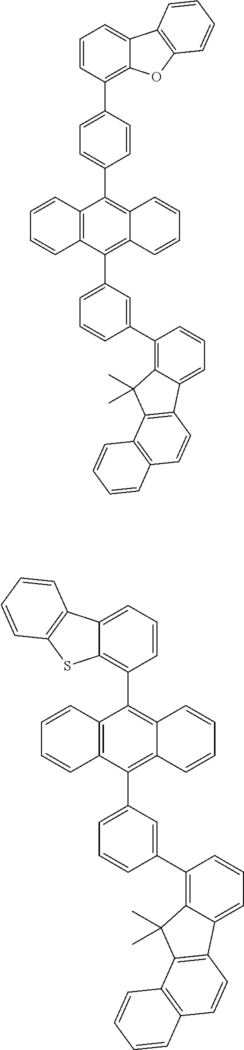 [SMILES: CC1(C)c2c(-c3cccc(-c4c5ccccc5c(-c5ccc(-c6cccc7c6oc6ccccc67)cc5)c5ccccc45)c3)cccc2-c2ccc3ccccc3c21 ; CC1(C)c2c(-c3cccc(-c4c5ccccc5c(-c5cccc6c5sc5ccccc56)c5ccccc45)c3)cccc2-c2ccc3ccccc3c21]

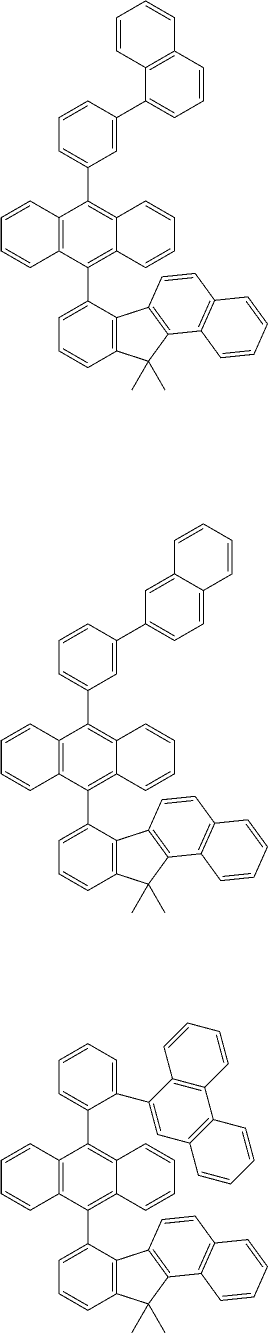

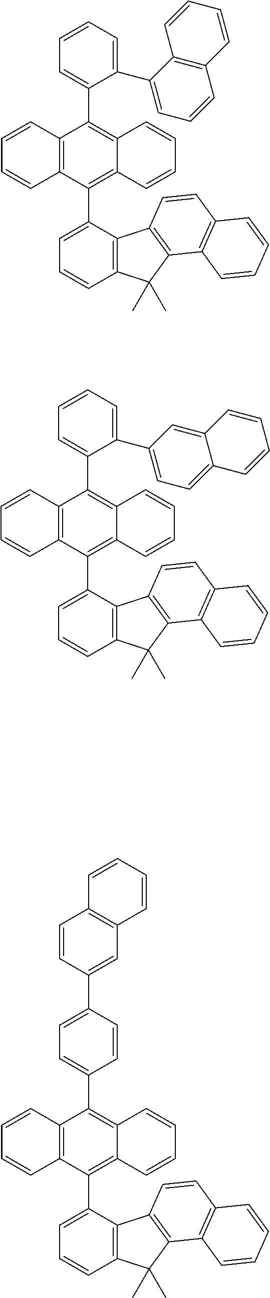

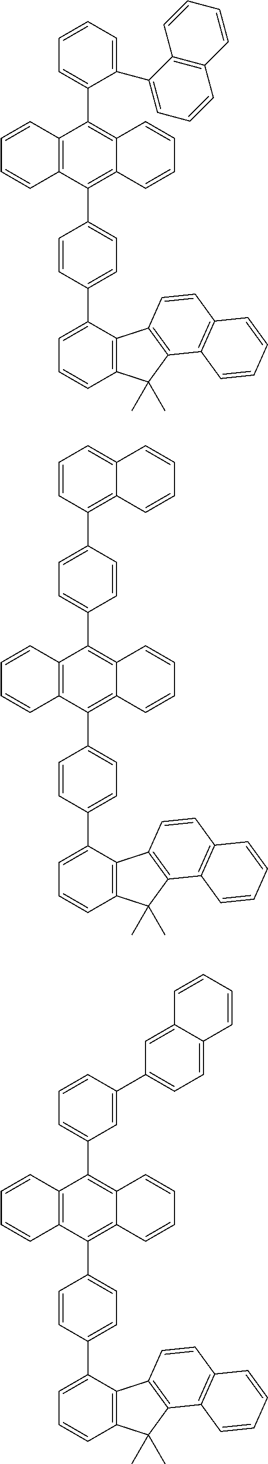

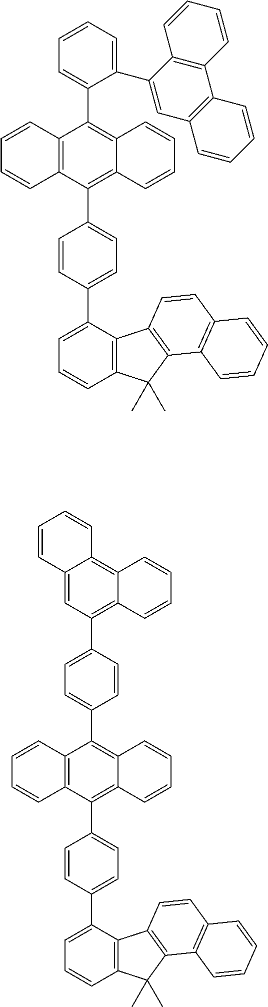

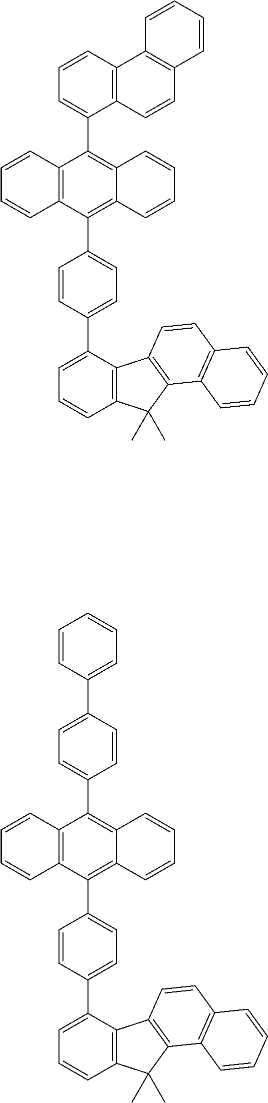

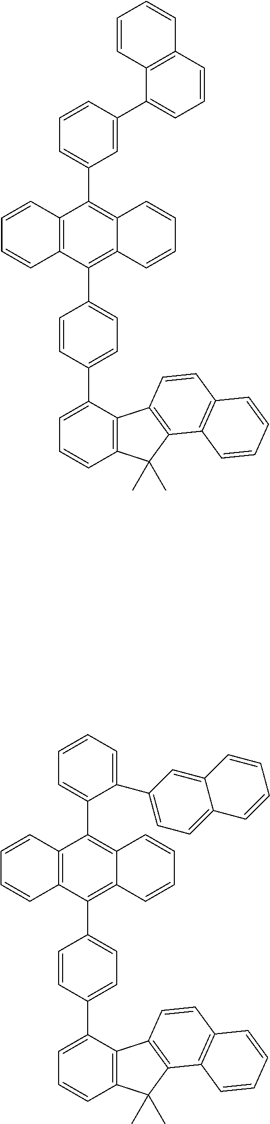

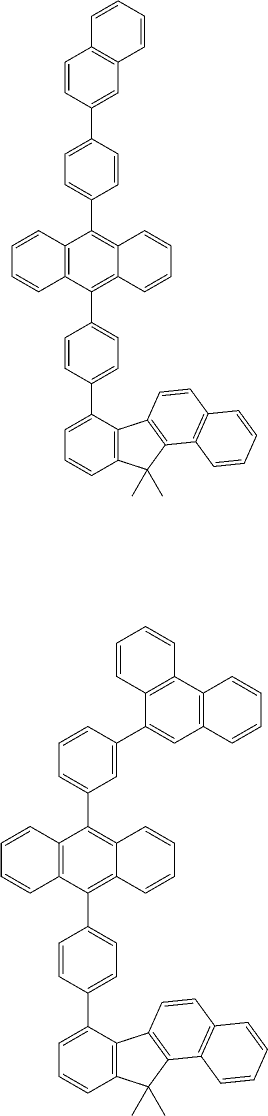

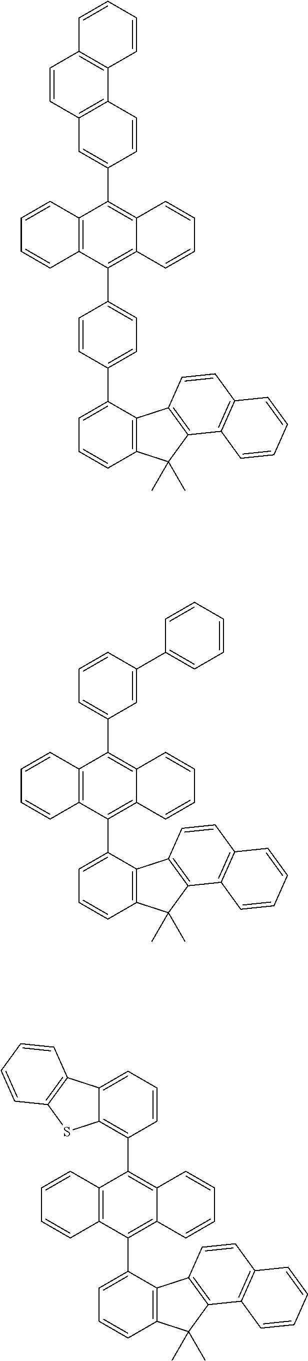

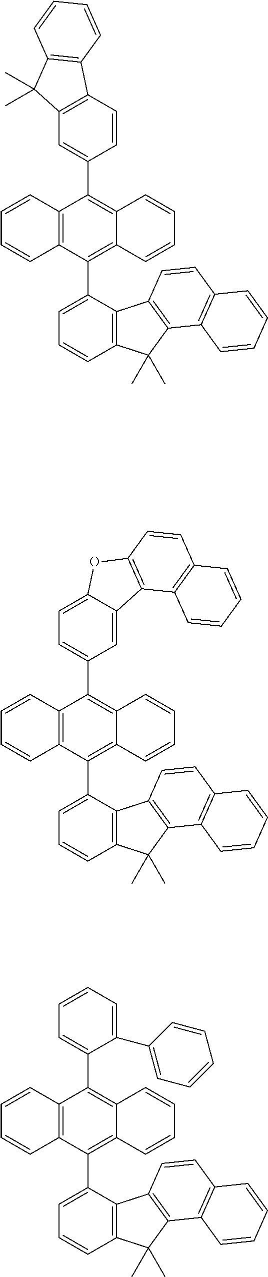

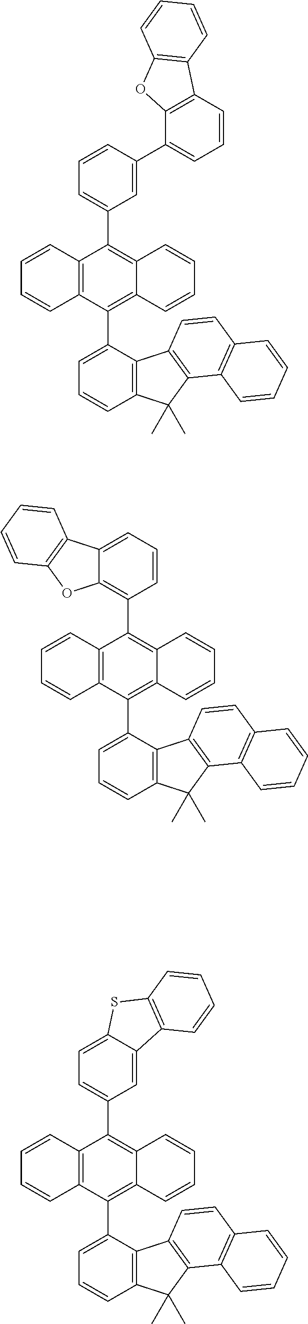 [SMILES: CC1(C)c2cccc(-c3c4ccccc4c(-c4cccc(-c5cccc6c5oc5ccccc56)c4)c4ccccc34)c2-c2ccc3ccccc3c21 ; CC1(C)c2cccc(-c3c4ccccc4c(-c4cccc5c4oc4ccccc45)c4ccccc34)c2-c2ccc3ccccc3c21 ; CC1(C)c2cccc(-c3c4ccccc4c(-c4ccc5sc6ccccc6c5c4)c4ccccc34)c2-c2ccc3ccccc3c21]

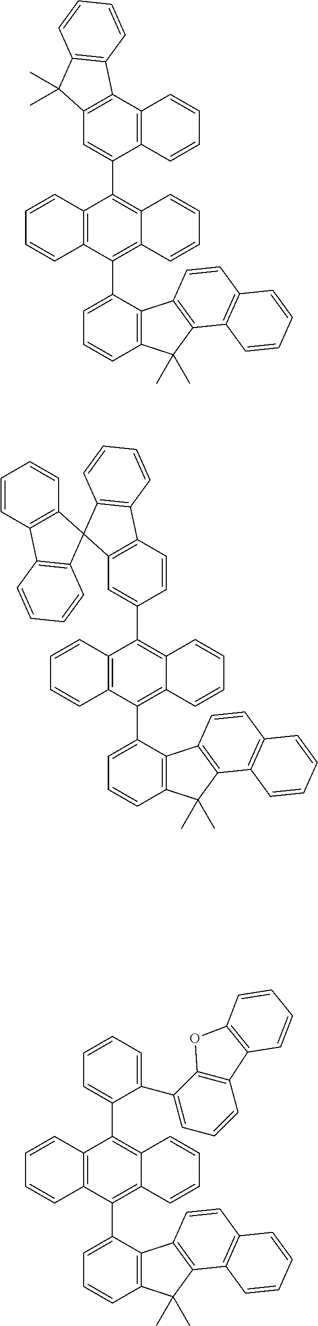

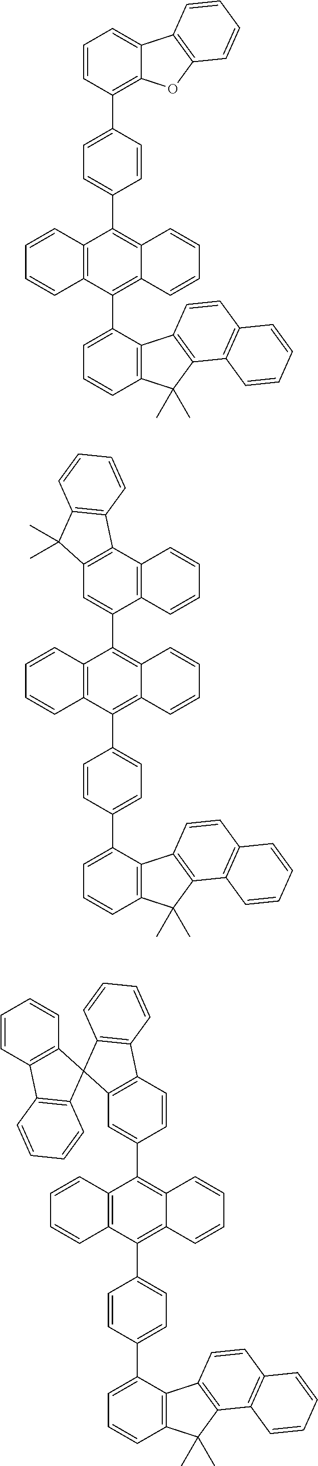

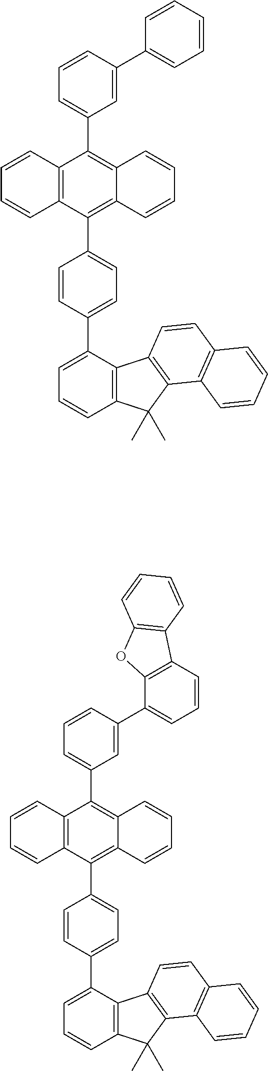

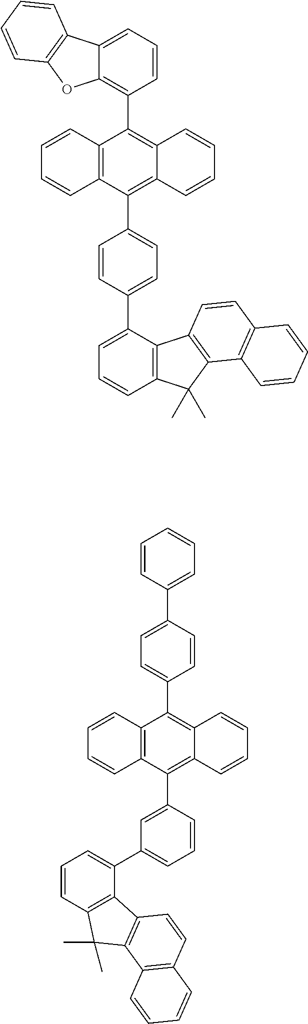

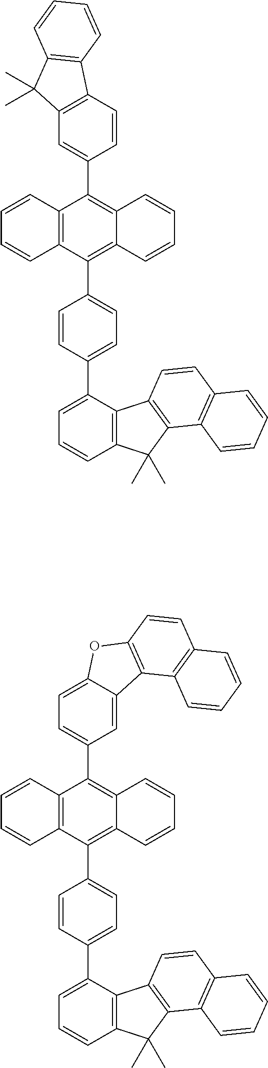

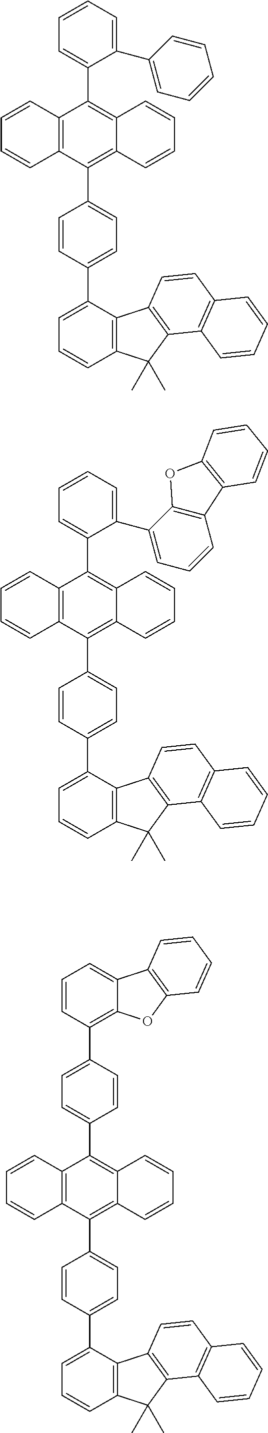

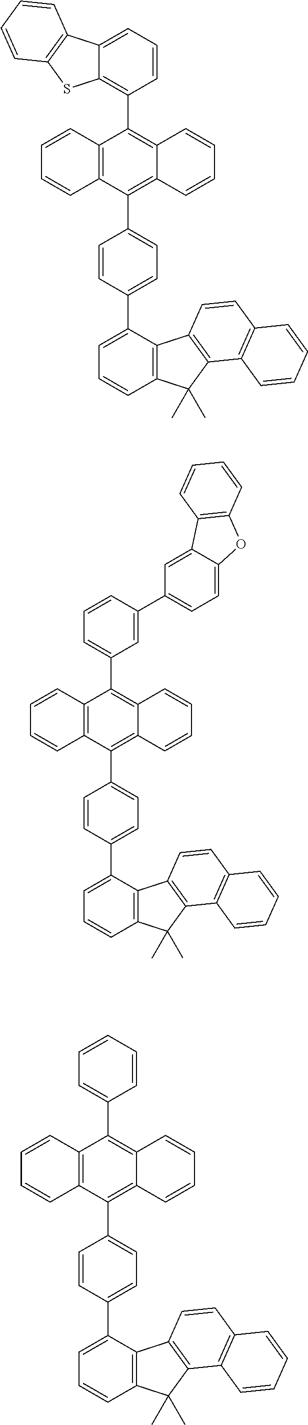

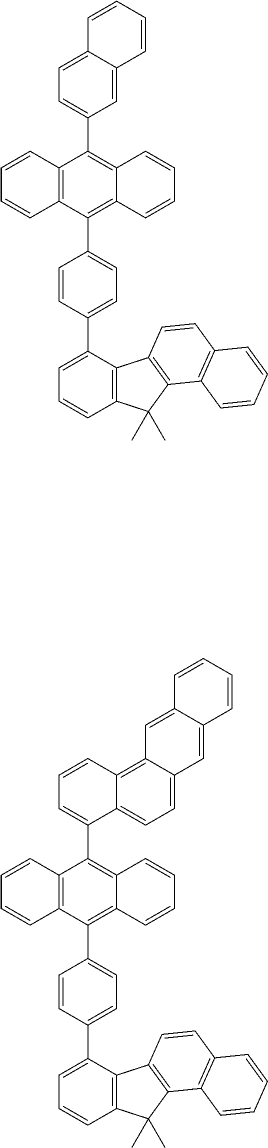

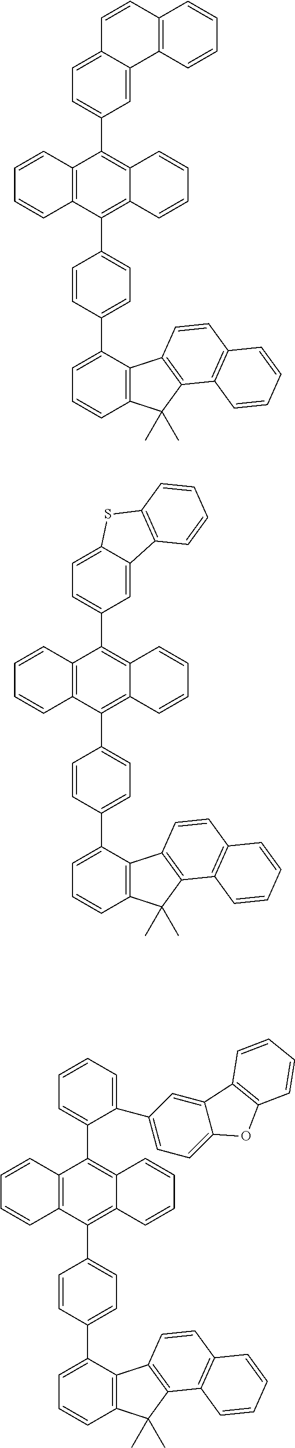

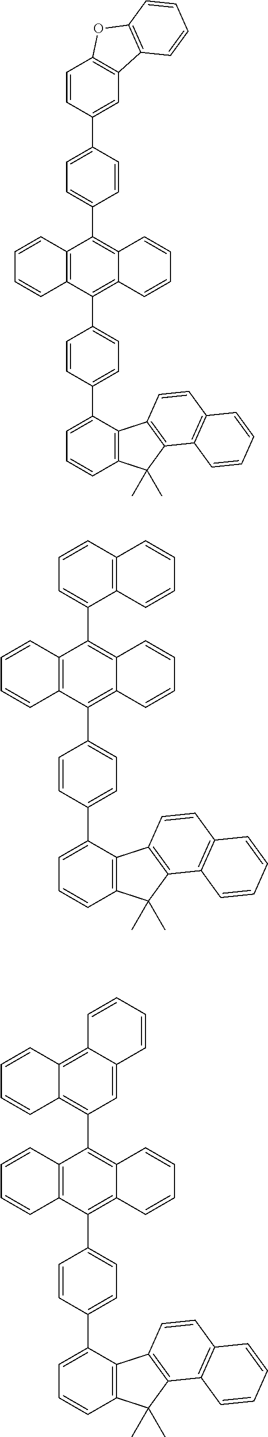

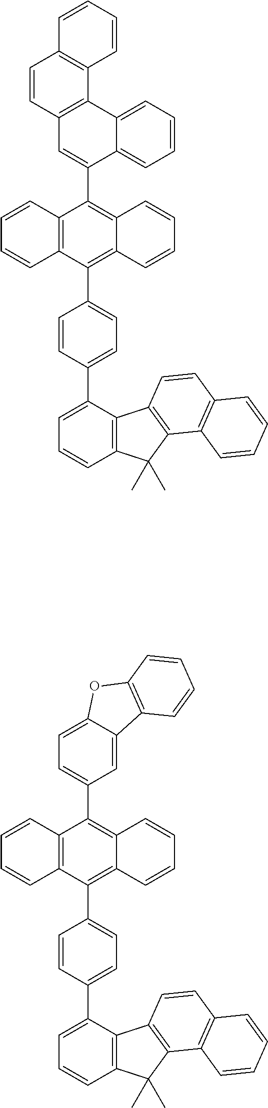

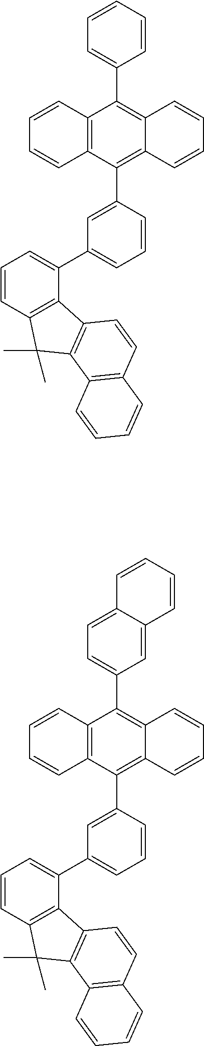

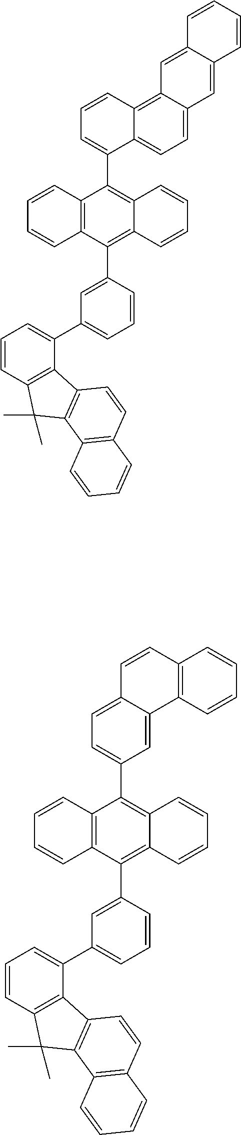

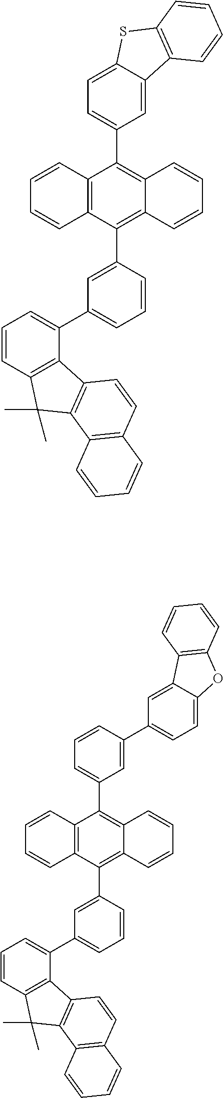 [SMILES: CC1(C)c2cccc(-c3cccc(-c4c5ccccc5c(-c5ccc6sc7ccccc7c6c5)c5ccccc45)c3)c2-c2ccc3ccccc3c21 ; CC1(C)c2cccc(-c3cccc(-c4c5ccccc5c(-c5cccc(-c6ccc7oc8ccccc8c7c6)c5)c5ccccc45)c3)c2-c2ccc3ccccc3c21]

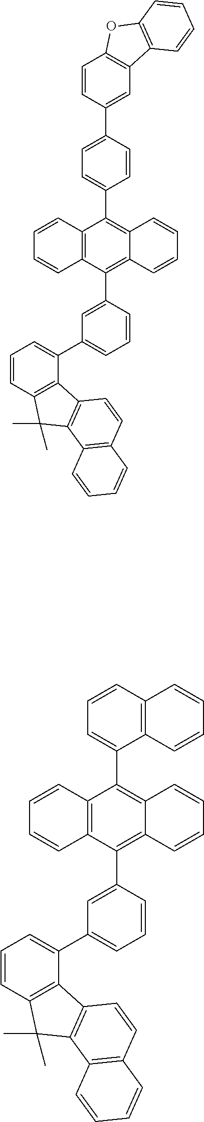

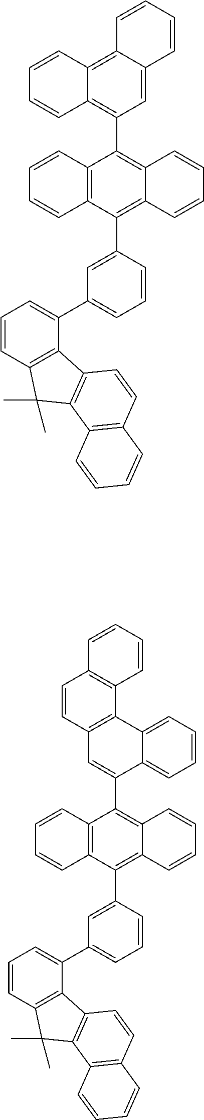

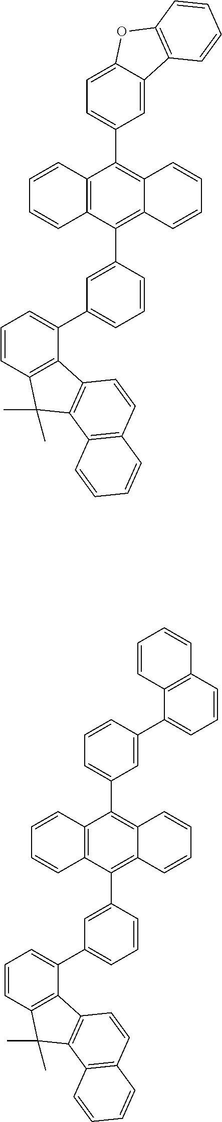

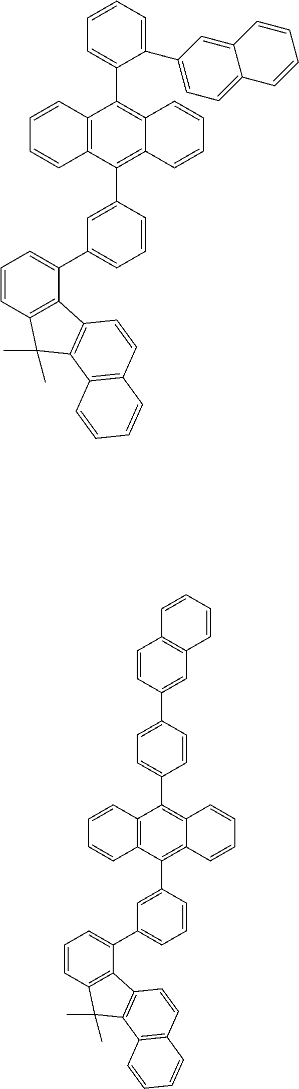

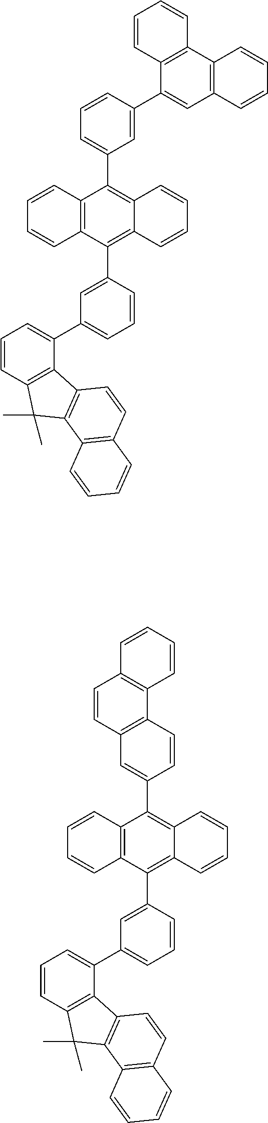

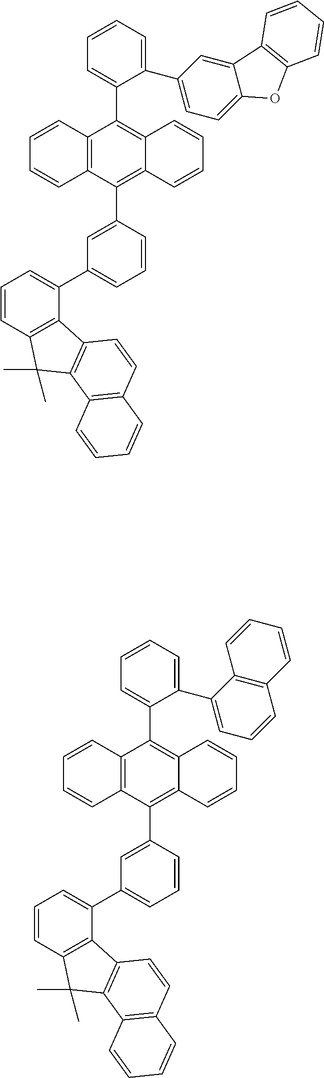

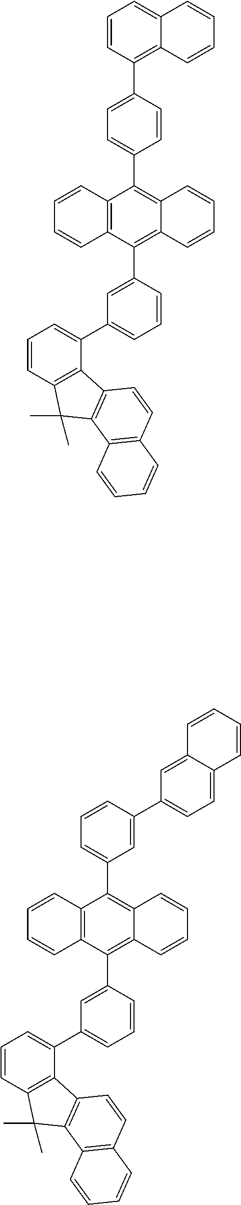

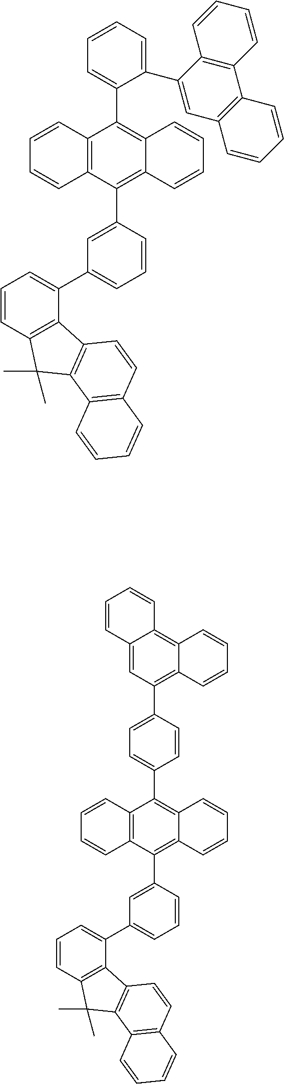

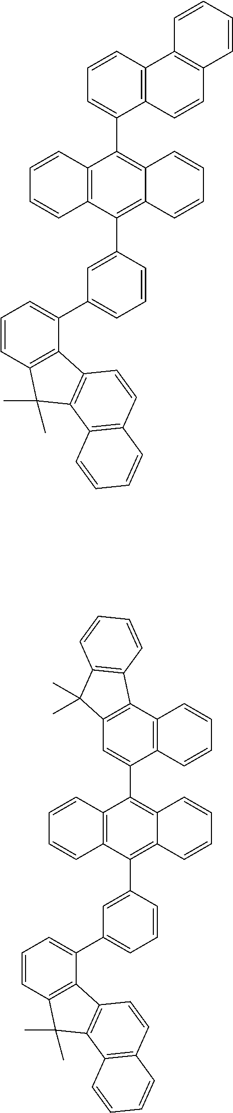

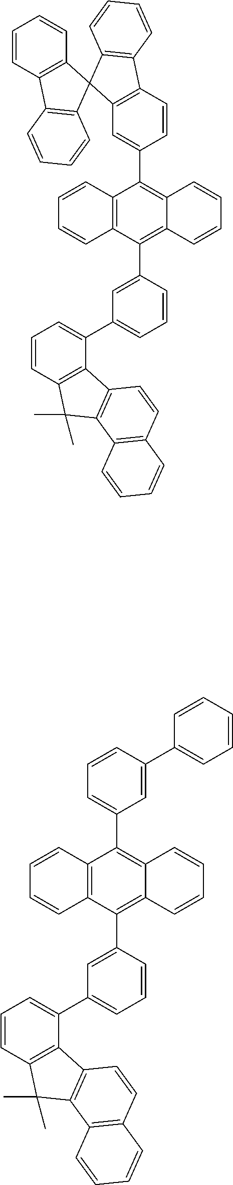

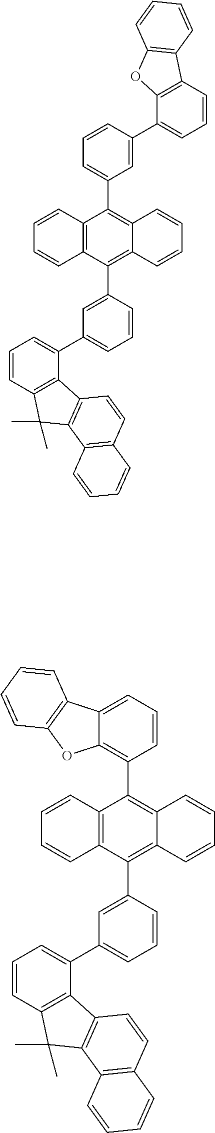 [SMILES: CC1(C)c2cccc(-c3cccc(-c4c5ccccc5c(-c5cccc(-c6cccc7c6oc6ccccc67)c5)c5ccccc45)c3)c2-c2ccc3ccccc3c21 ; CC1(C)c2cccc(-c3cccc(-c4c5ccccc5c(-c5cccc6c5oc5ccccc56)c5ccccc45)c3)c2-c2ccc3ccccc3c21]

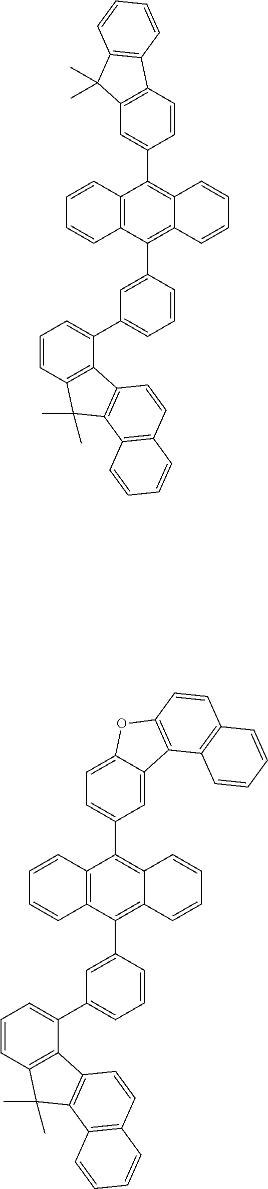

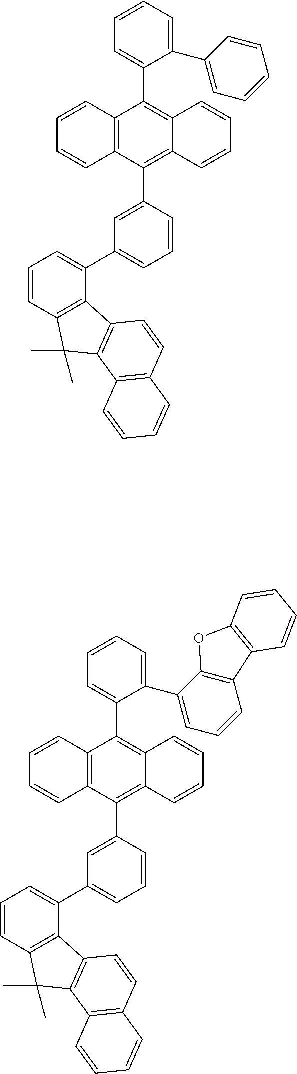

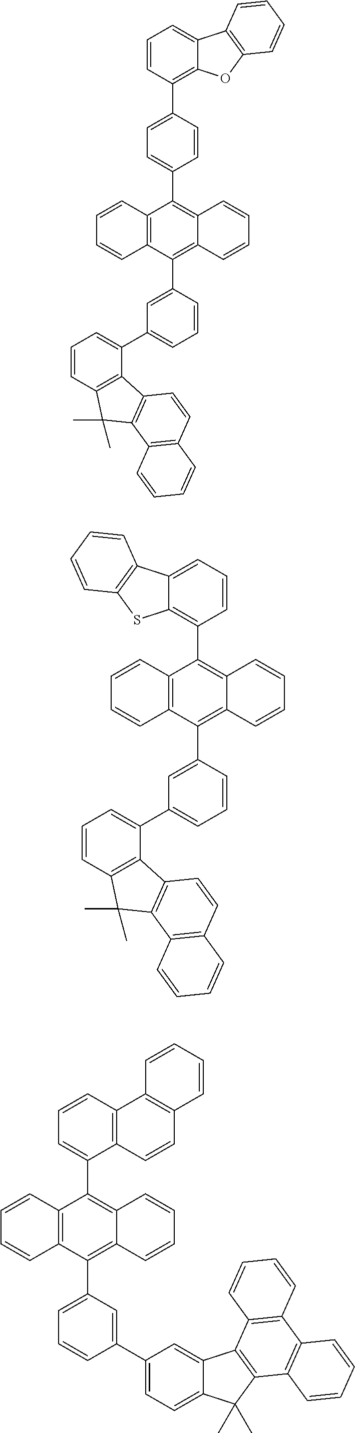

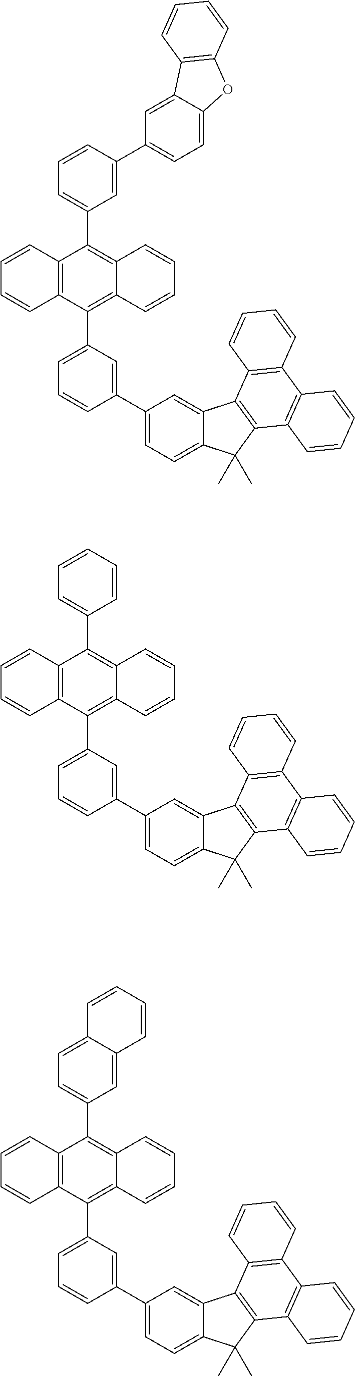

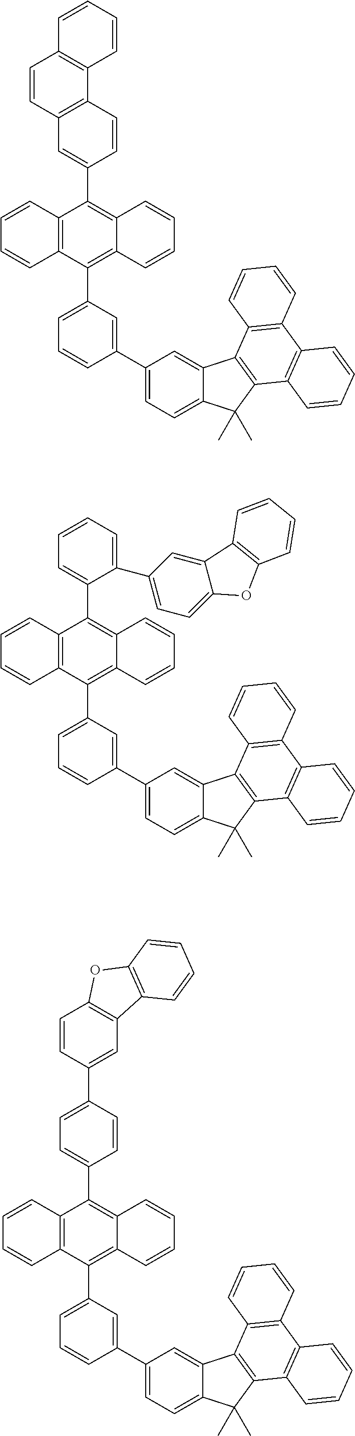

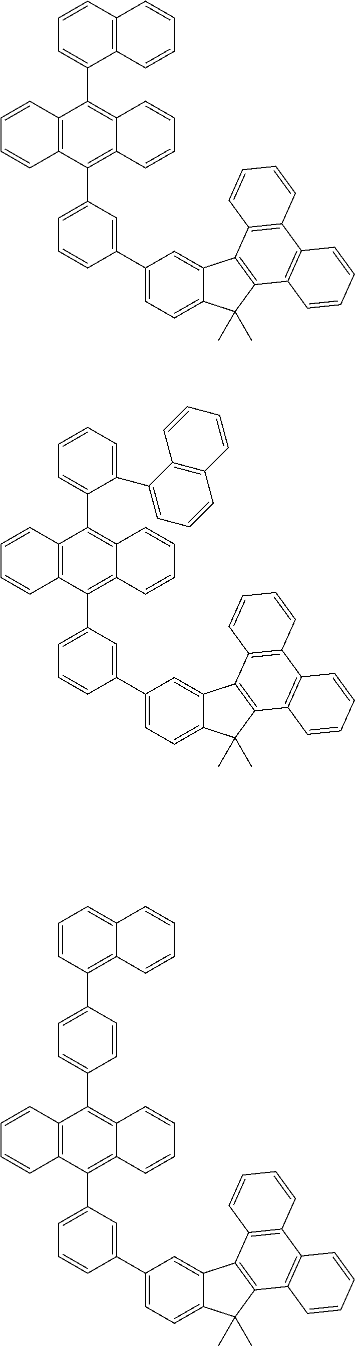

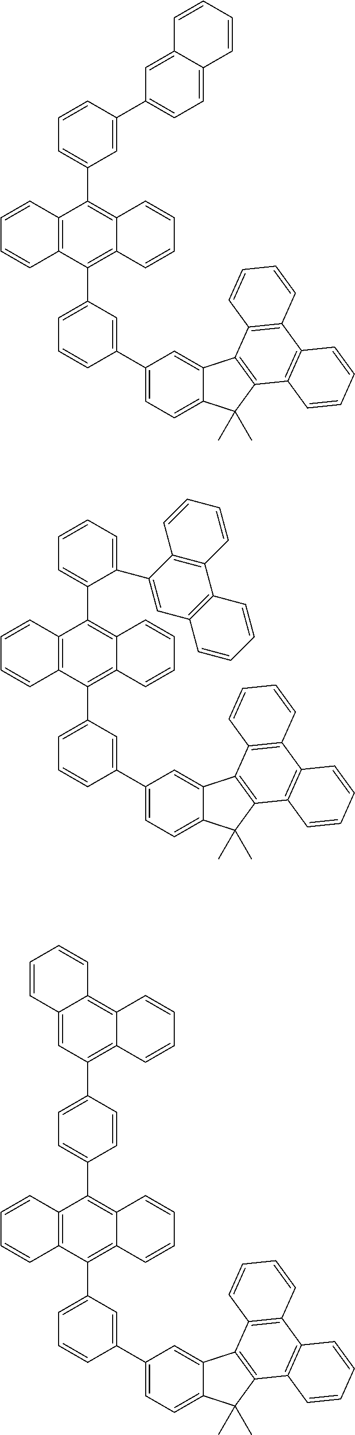

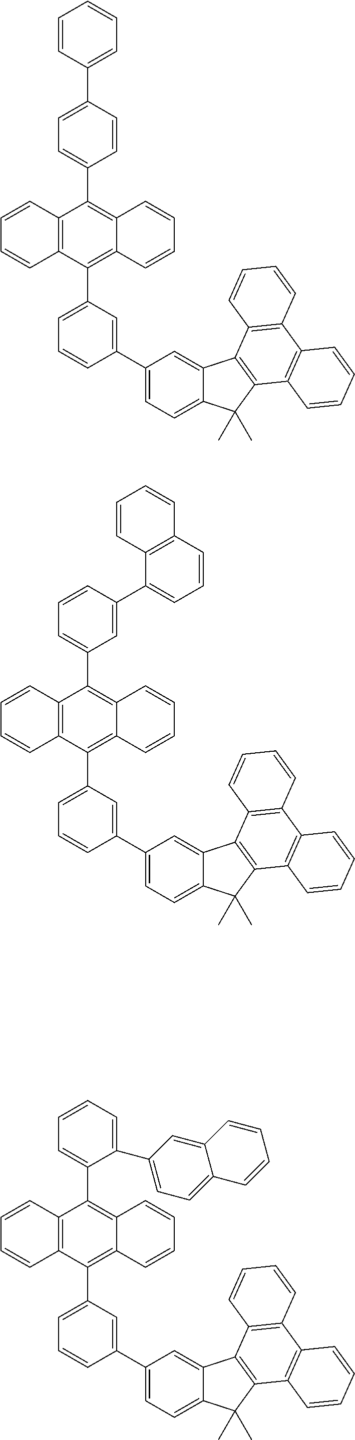

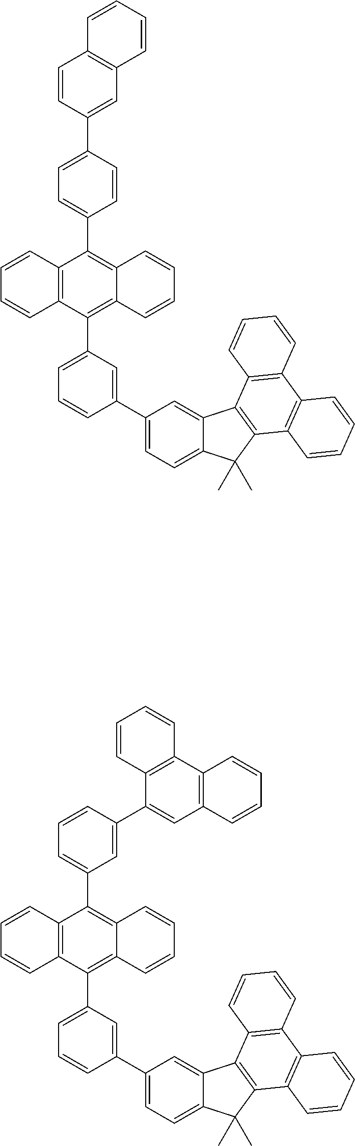

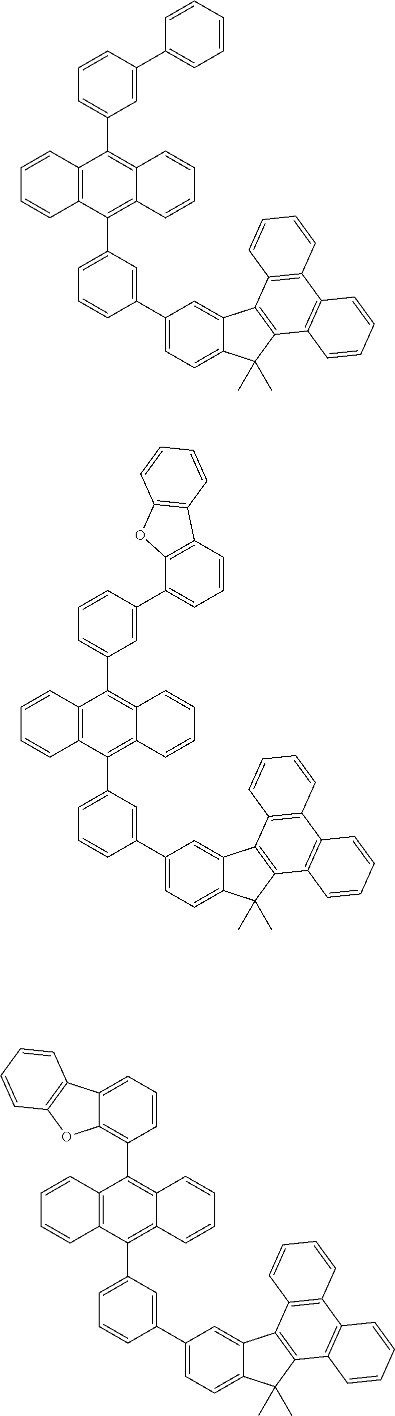

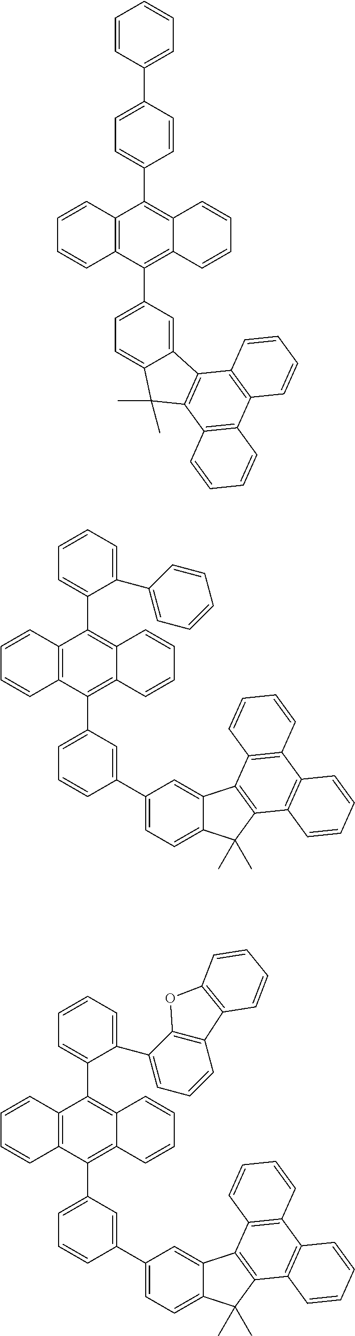

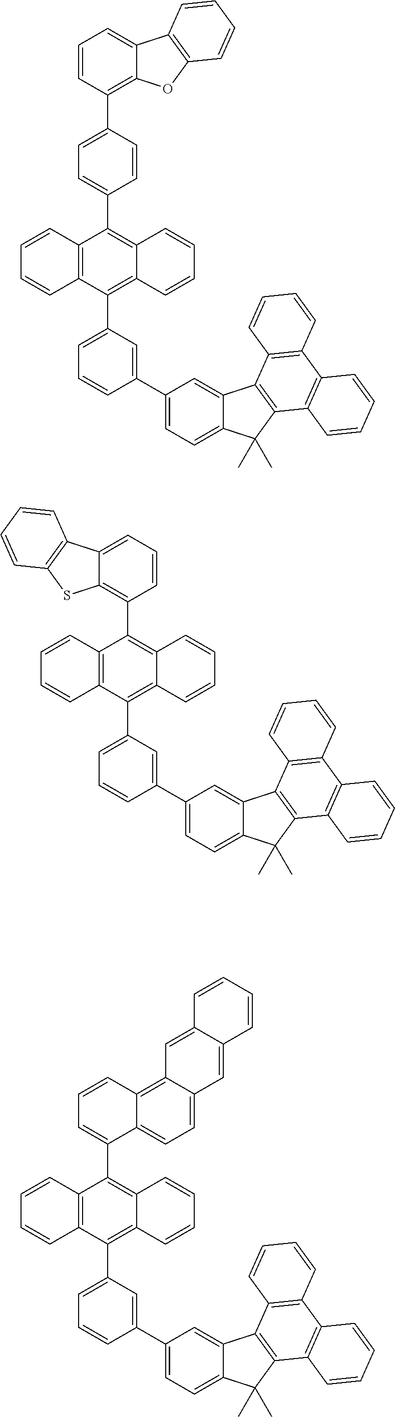

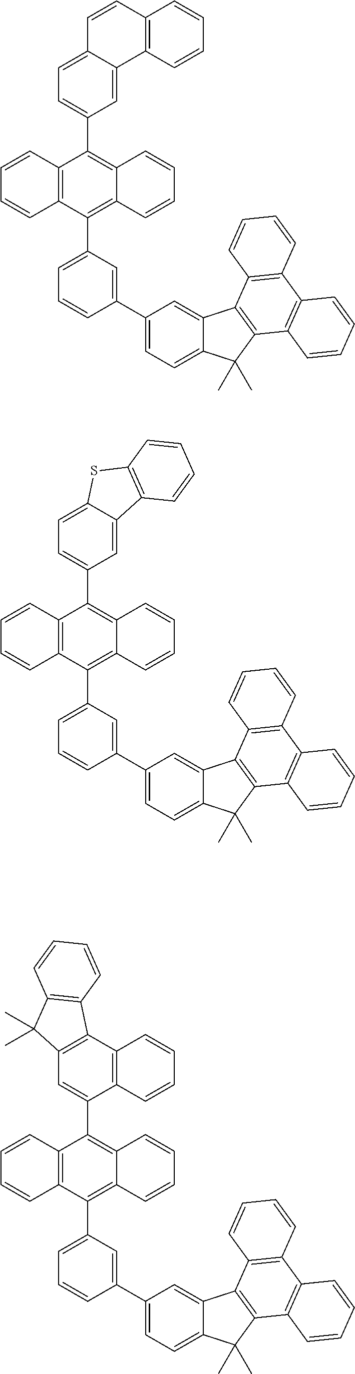

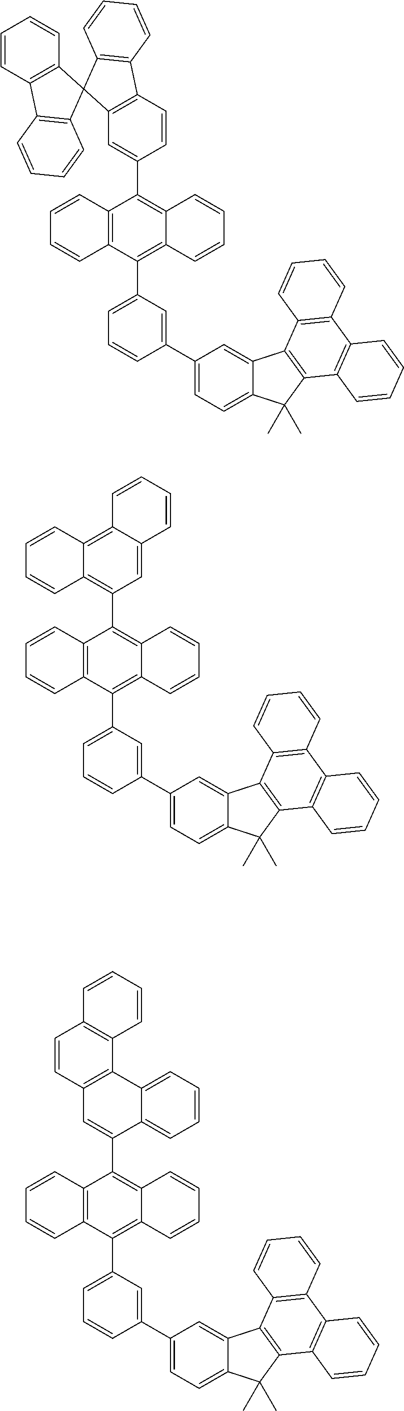

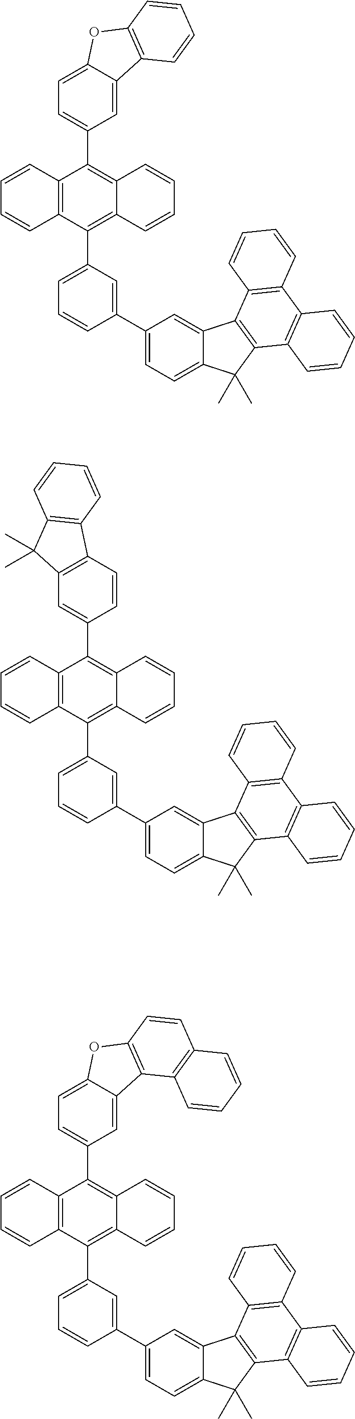

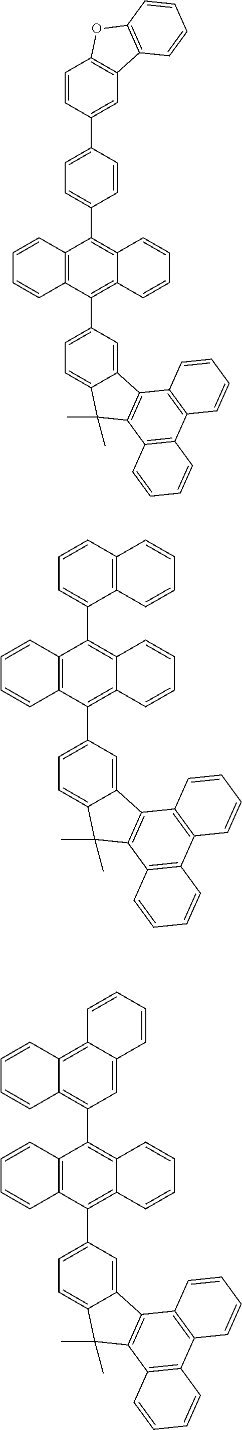

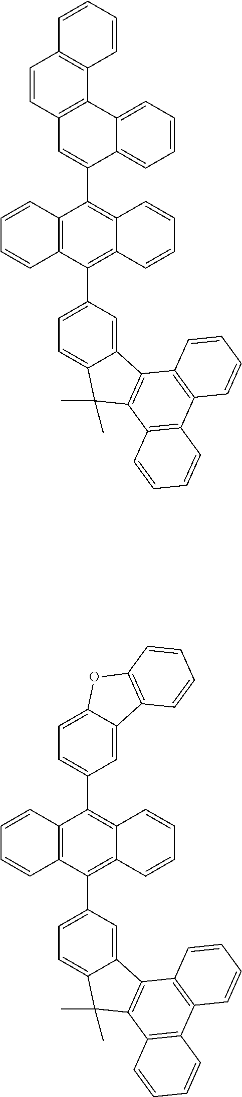

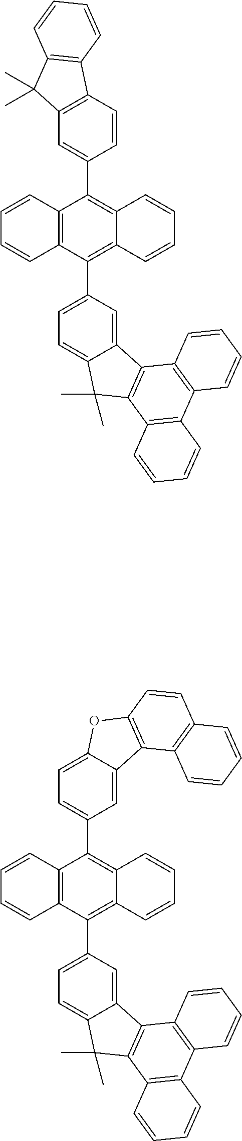

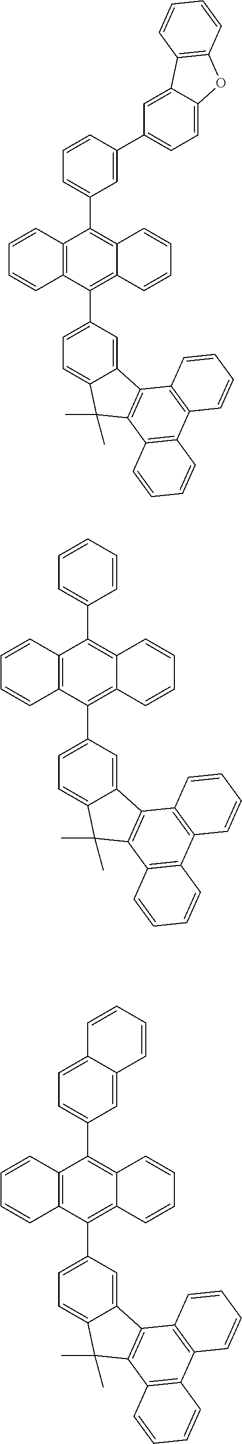

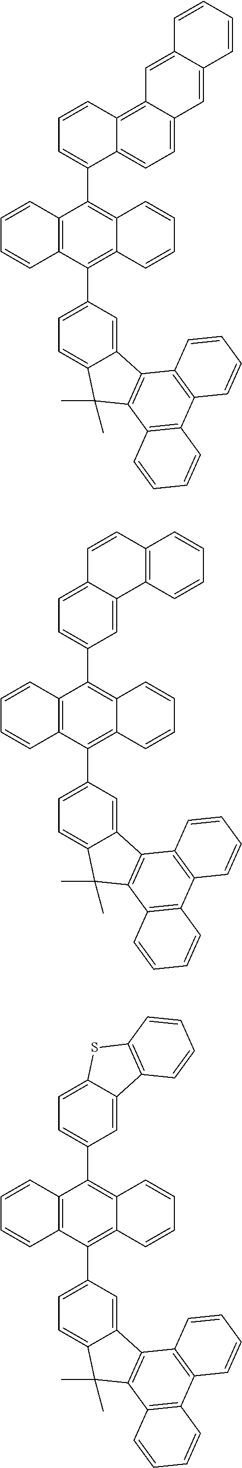

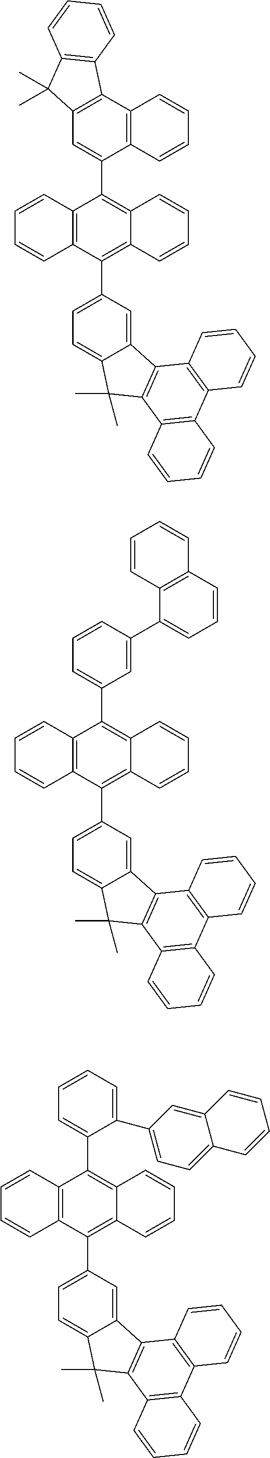

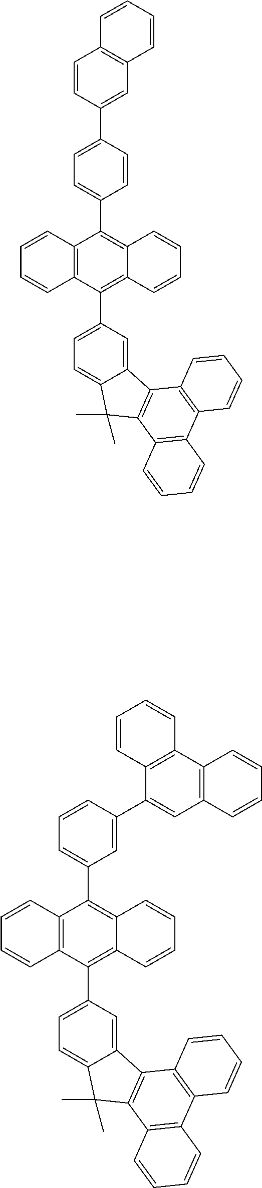

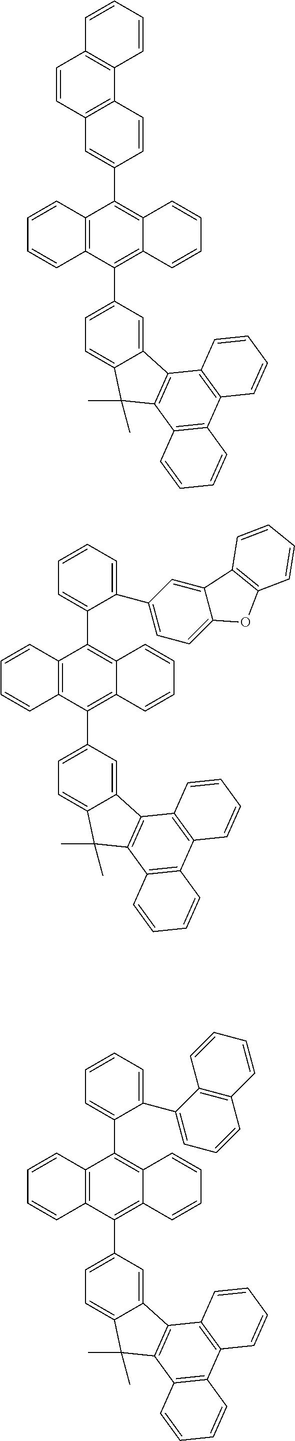

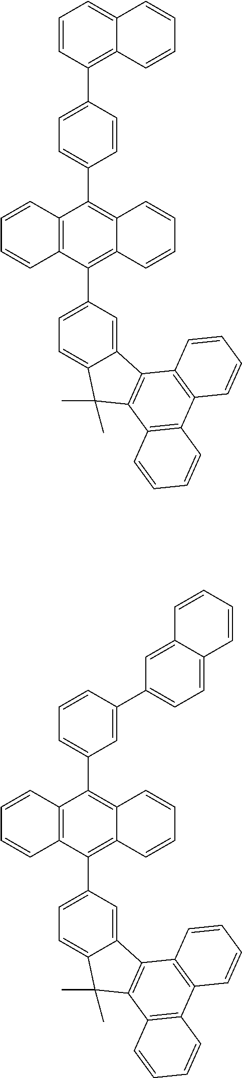

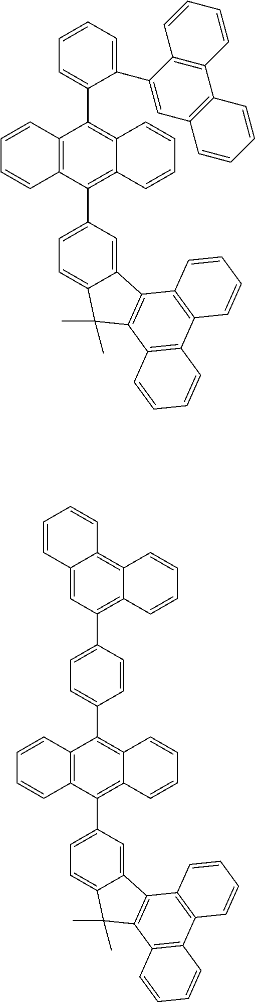

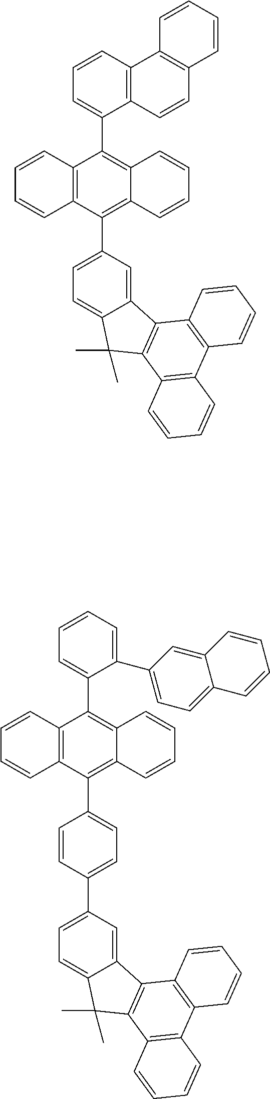

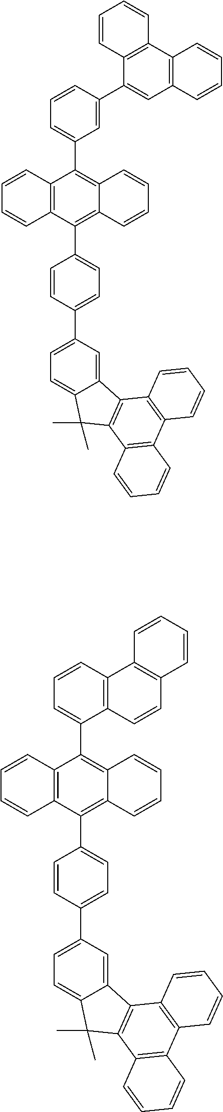

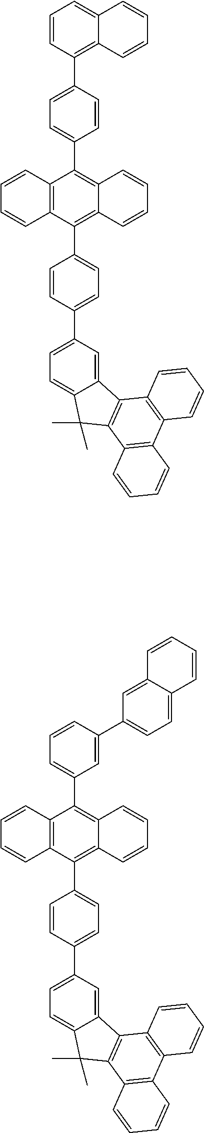

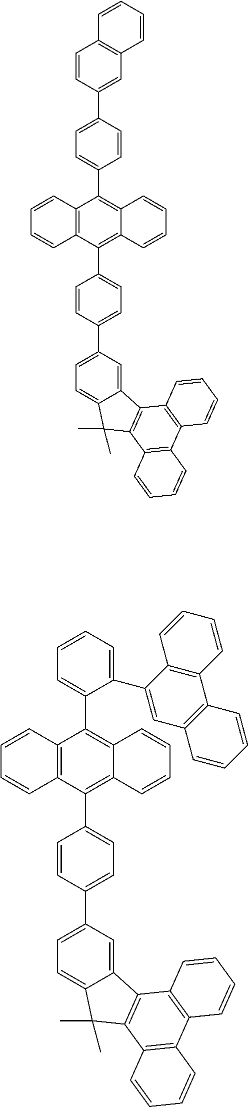

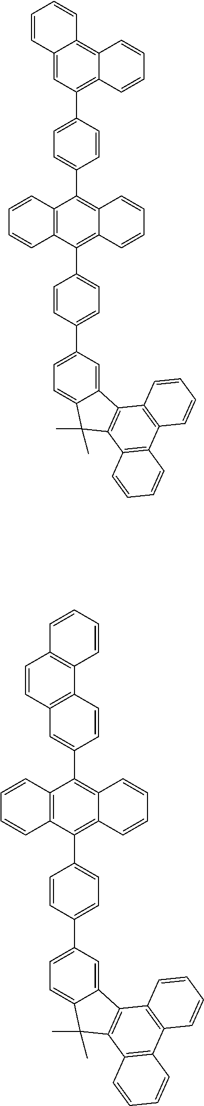

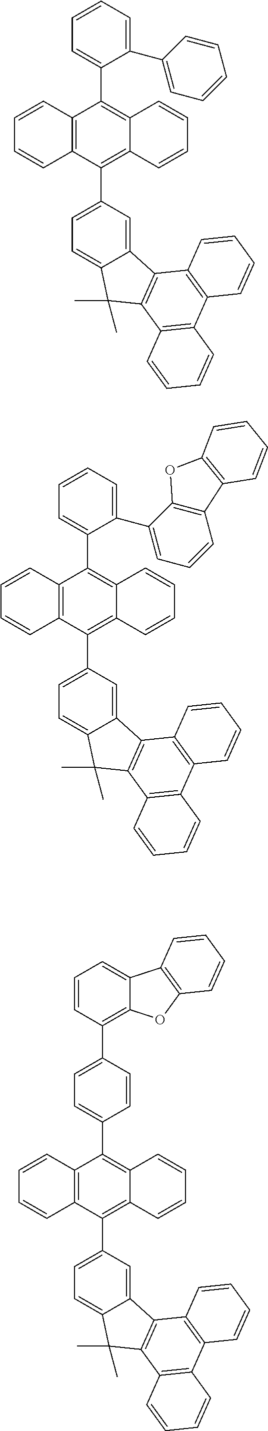

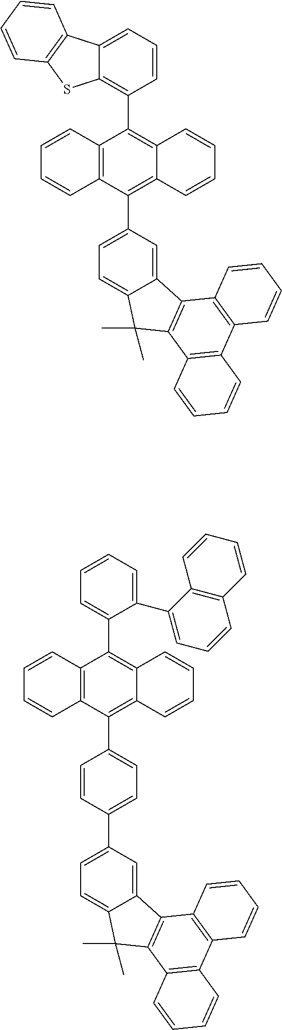

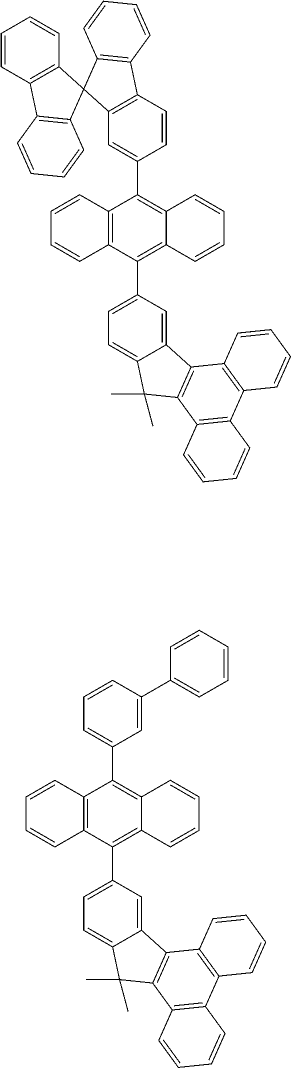

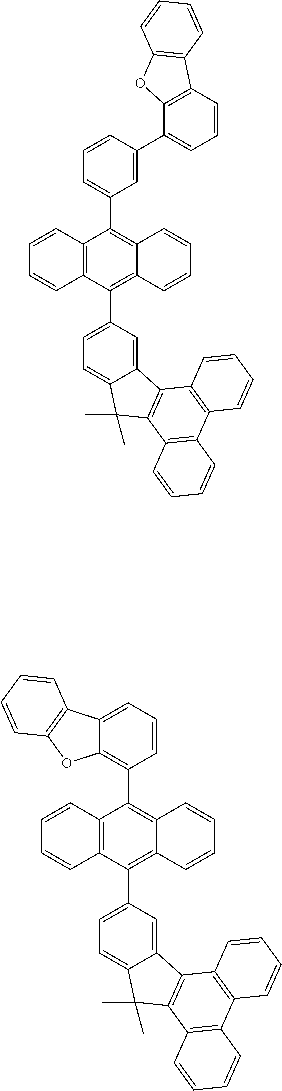 [SMILES: CC1(C)c2ccc(-c3c4ccccc4c(-c4cccc(-c5cccc6c5oc5ccccc56)c4)c4ccccc34)cc2-c2c1c1ccccc1c1ccccc21 ; CC1(C)c2ccc(-c3c4ccccc4c(-c4cccc5c4oc4ccccc45)c4ccccc34)cc2-c2c1c1ccccc1c1ccccc21]

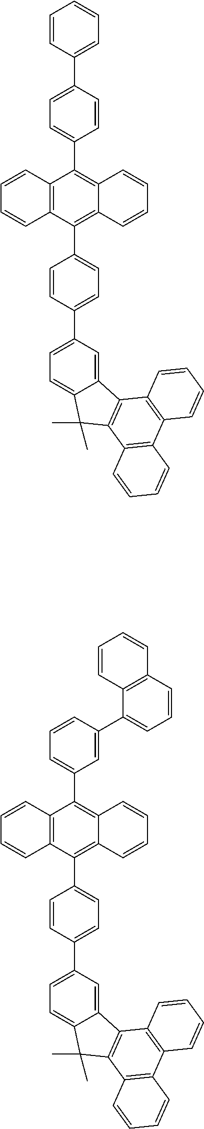

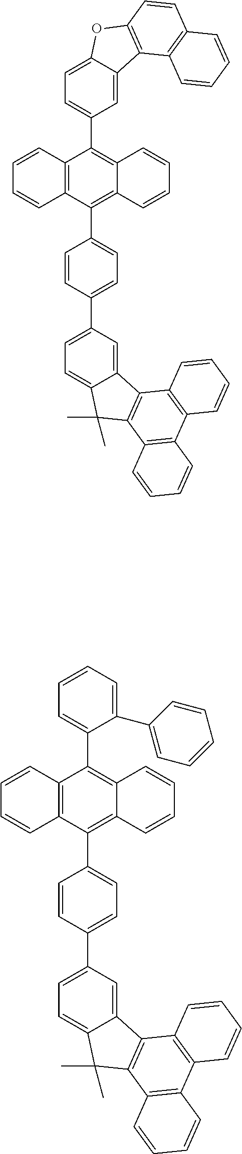

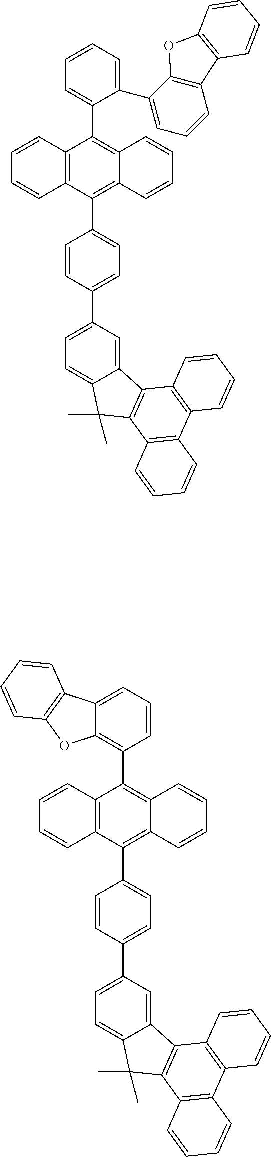 [SMILES: CC1(C)c2ccc(-c3ccc(-c4c5ccccc5c(-c5ccccc5-c5cccc6c5oc5ccccc56)c5ccccc45)cc3)cc2-c2c1c1ccccc1c1ccccc21 ; CC1(C)c2ccc(-c3ccc(-c4c5ccccc5c(-c5cccc6c5oc5ccccc56)c5ccccc45)cc3)cc2-c2c1c1ccccc1c1ccccc21]

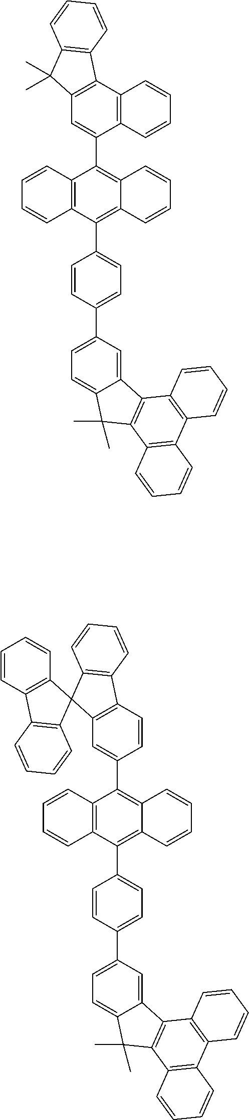

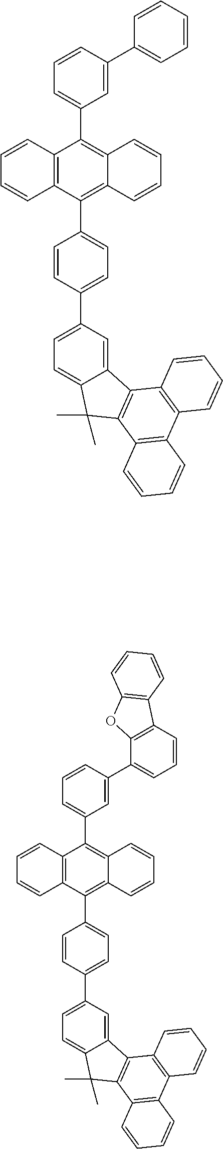

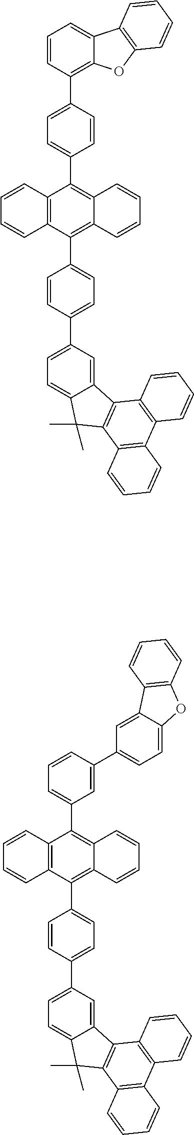 [SMILES: CC1(C)c2ccc(-c3ccc(-c4c5ccccc5c(-c5ccc(-c6cccc7c6oc6ccccc67)cc5)c5ccccc45)cc3)cc2-c2c1c1ccccc1c1ccccc21 ; CC1(C)c2ccc(-c3ccc(-c4c5ccccc5c(-c5cccc(-c6ccc7oc8ccccc8c7c6)c5)c5ccccc45)cc3)cc2-c2c1c1ccccc1c1ccccc21]

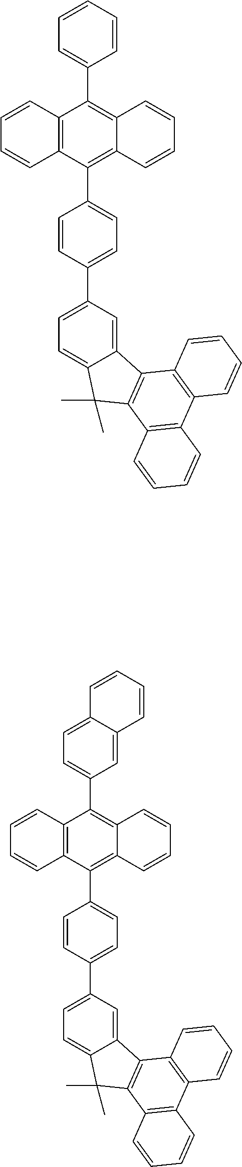

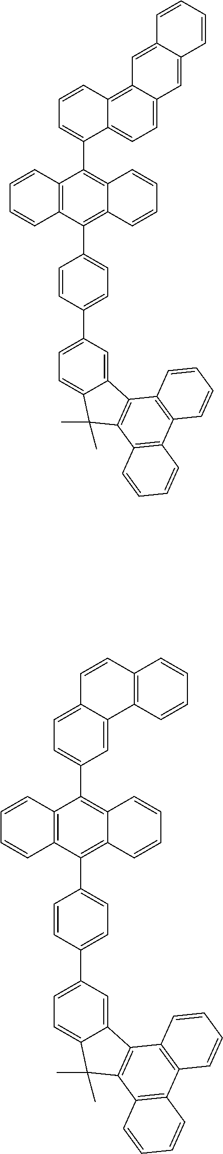

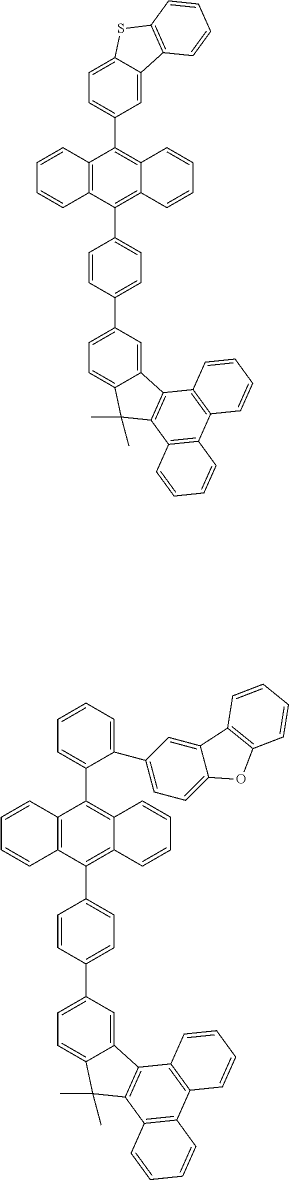 [SMILES: CC1(C)c2ccc(-c3ccc(-c4c5ccccc5c(-c5ccc6sc7ccccc7c6c5)c5ccccc45)cc3)cc2-c2c1c1ccccc1c1ccccc21 ; CC1(C)c2ccc(-c3ccc(-c4c5ccccc5c(-c5ccccc5-c5ccc6oc7ccccc7c6c5)c5ccccc45)cc3)cc2-c2c1c1ccccc1c1ccccc21]

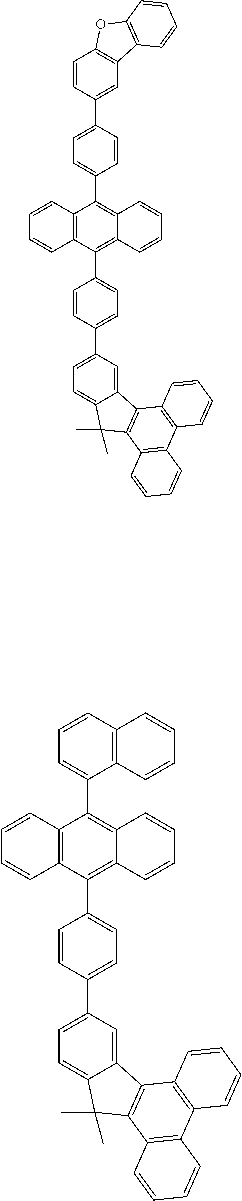

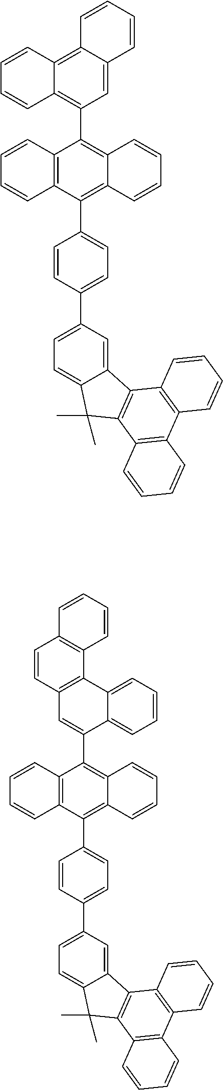

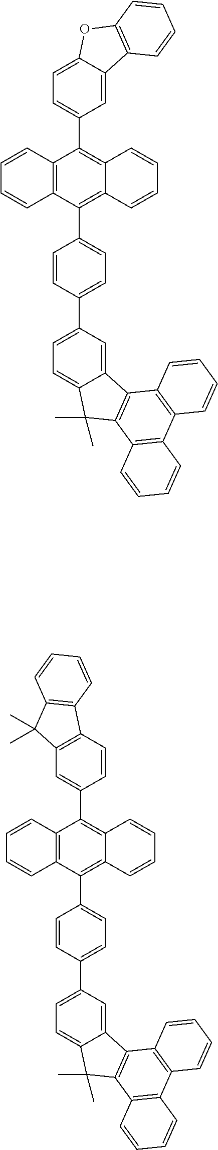

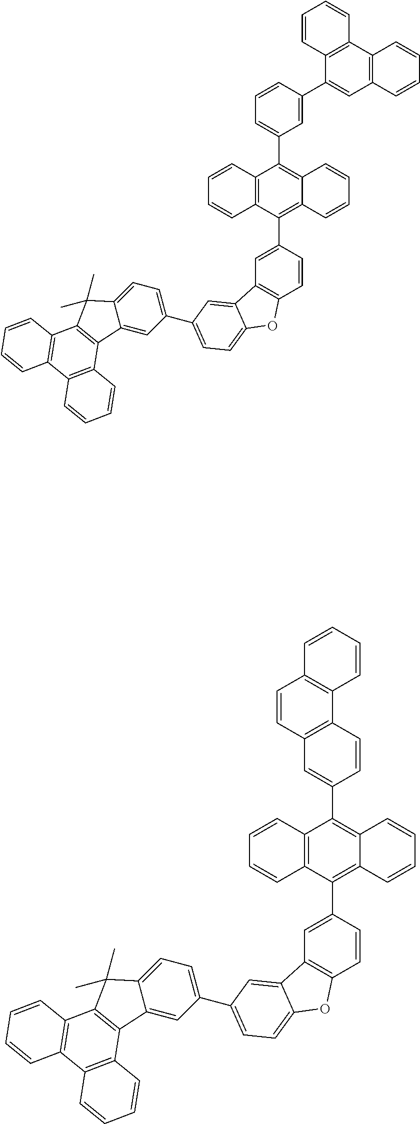 [SMILES: CC1(C)c2ccc(-c3ccc4oc5ccc(-c6c7ccccc7c(-c7cccc(-c8cc9ccccc9c9ccccc89)c7)c7ccccc67)cc5c4c3)cc2-c2c1c1ccccc1c1ccccc21 ; CC1(C)c2ccc(-c3ccc4oc5ccc(-c6c7ccccc7c(-c7ccc8c(ccc9ccccc98)c7)c7ccccc67)cc5c4c3)cc2-c2c1c1ccccc1c1ccccc21]

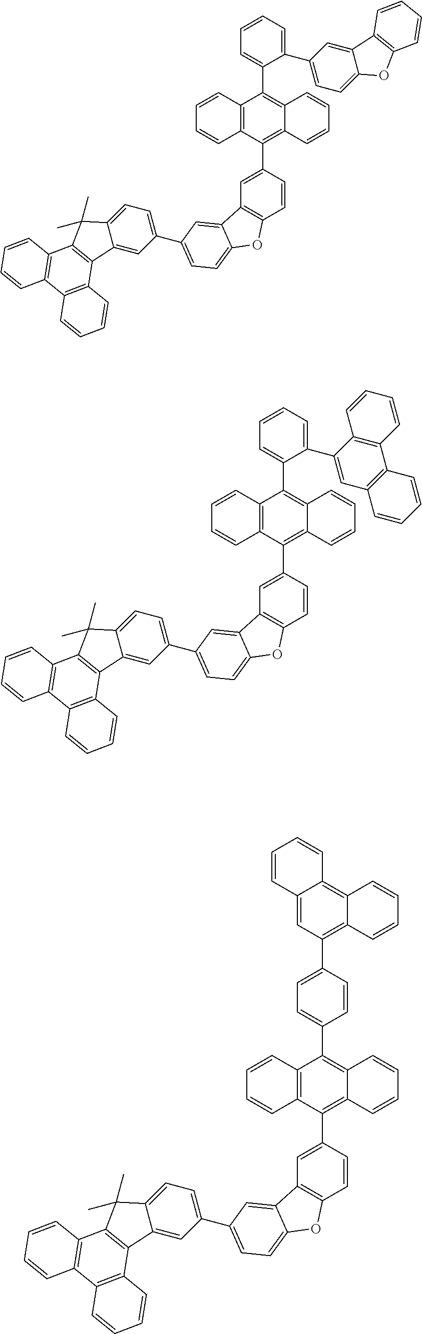 [SMILES: CC1(C)c2ccc(-c3ccc4oc5ccc(-c6c7ccccc7c(-c7ccccc7-c7ccc8oc9ccccc9c8c7)c7ccccc67)cc5c4c3)cc2-c2c1c1ccccc1c1ccccc21 ; CC1(C)c2ccc(-c3ccc4oc5ccc(-c6c7ccccc7c(-c7ccccc7-c7cc8ccccc8c8ccccc78)c7ccccc67)cc5c4c3)cc2-c2c1c1ccccc1c1ccccc21 ; CC1(C)c2ccc(-c3ccc4oc5ccc(-c6c7ccccc7c(-c7ccc(-c8cc9ccccc9c9ccccc89)cc7)c7ccccc67)cc5c4c3)cc2-c2c1c1ccccc1c1ccccc21]

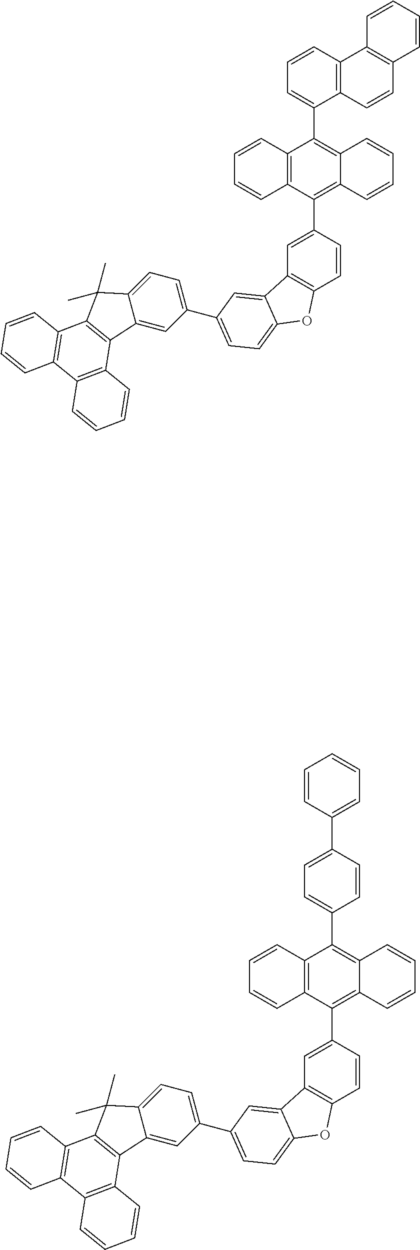 [SMILES: CC1(C)c2ccc(-c3ccc4oc5ccc(-c6c7ccccc7c(-c7cccc8c7ccc7ccccc78)c7ccccc67)cc5c4c3)cc2-c2c1c1ccccc1c1ccccc21 ; CC1(C)c2ccc(-c3ccc4oc5ccc(-c6c7ccccc7c(-c7ccc(-c8ccccc8)cc7)c7ccccc67)cc5c4c3)cc2-c2c1c1ccccc1c1ccccc21]

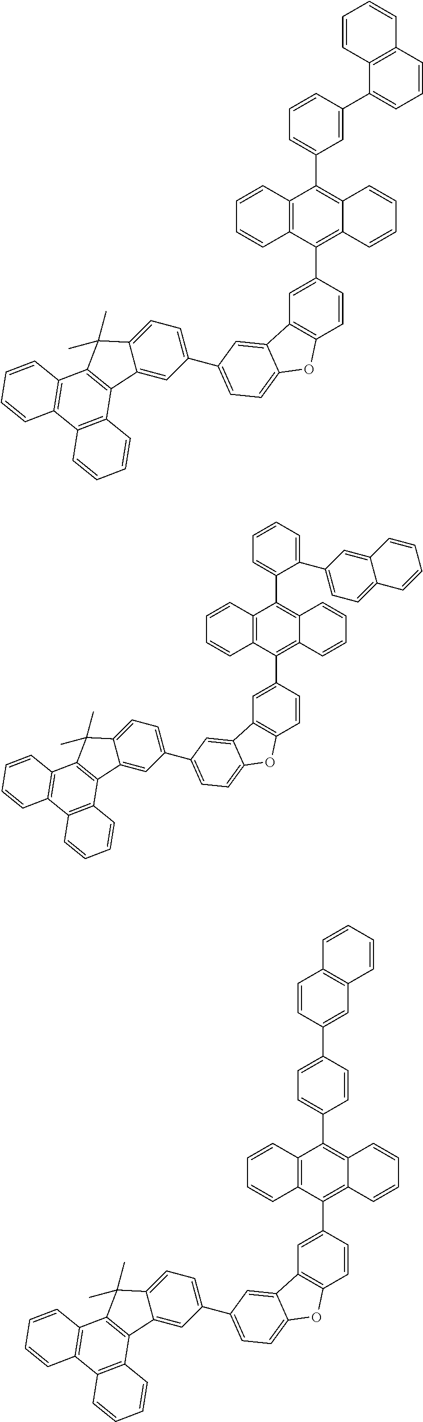 [SMILES: CC1(C)c2ccc(-c3ccc4oc5ccc(-c6c7ccccc7c(-c7cccc(-c8cccc9ccccc89)c7)c7ccccc67)cc5c4c3)cc2-c2c1c1ccccc1c1ccccc21 ; CC1(C)c2ccc(-c3ccc4oc5ccc(-c6c7ccccc7c(-c7ccccc7-c7ccc8ccccc8c7)c7ccccc67)cc5c4c3)cc2-c2c1c1ccccc1c1ccccc21 ; CC1(C)c2ccc(-c3ccc4oc5ccc(-c6c7ccccc7c(-c7ccc(-c8ccc9ccccc9c8)cc7)c7ccccc67)cc5c4c3)cc2-c2c1c1ccccc1c1ccccc21]

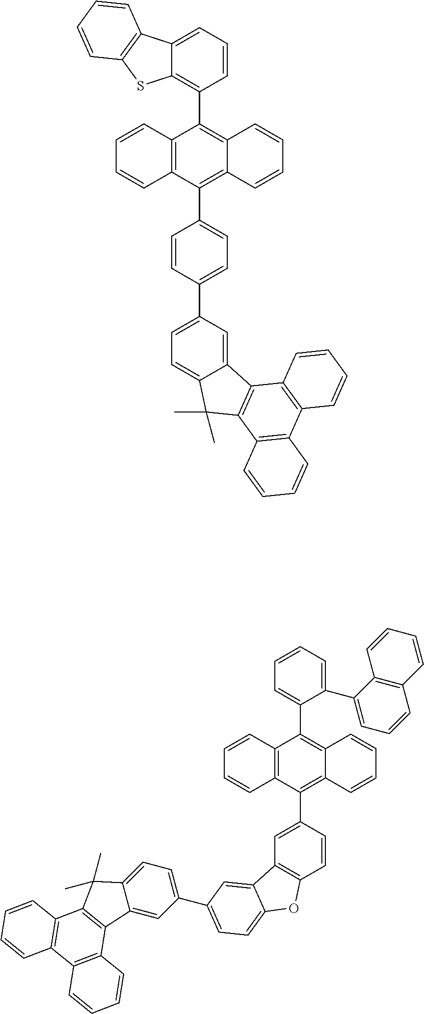 [SMILES: CC1(C)c2ccc(-c3ccc(-c4c5ccccc5c(-c5cccc6c5sc5ccccc56)c5ccccc45)cc3)cc2-c2c1c1ccccc1c1ccccc21 ; CC1(C)c2ccc(-c3ccc4oc5ccc(-c6c7ccccc7c(-c7ccccc7-c7cccc8ccccc78)c7ccccc67)cc5c4c3)cc2-c2c1c1ccccc1c1ccccc21]

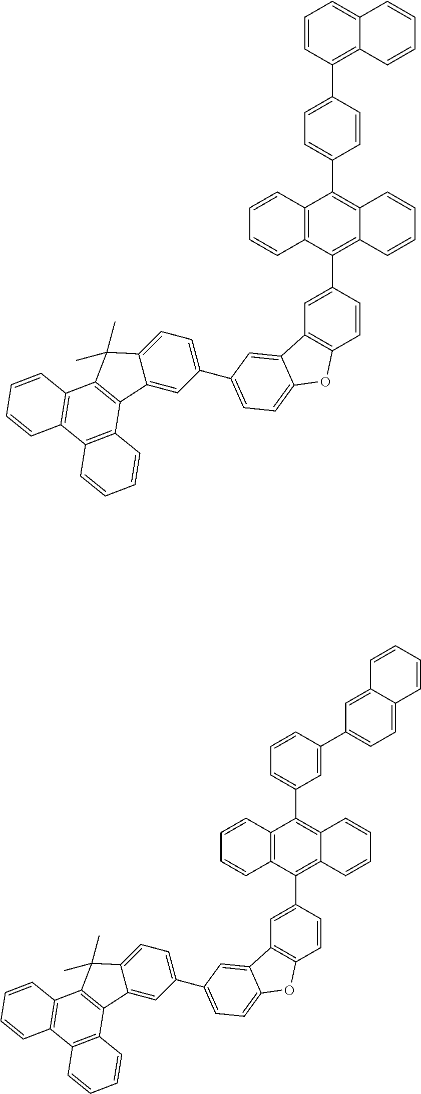 [SMILES: CC1(C)c2ccc(-c3ccc4oc5ccc(-c6c7ccccc7c(-c7ccc(-c8cccc9ccccc89)cc7)c7ccccc67)cc5c4c3)cc2-c2c1c1ccccc1c1ccccc21 ; CC1(C)c2ccc(-c3ccc4oc5ccc(-c6c7ccccc7c(-c7cccc(-c8ccc9ccccc9c8)c7)c7ccccc67)cc5c4c3)cc2-c2c1c1ccccc1c1ccccc21]

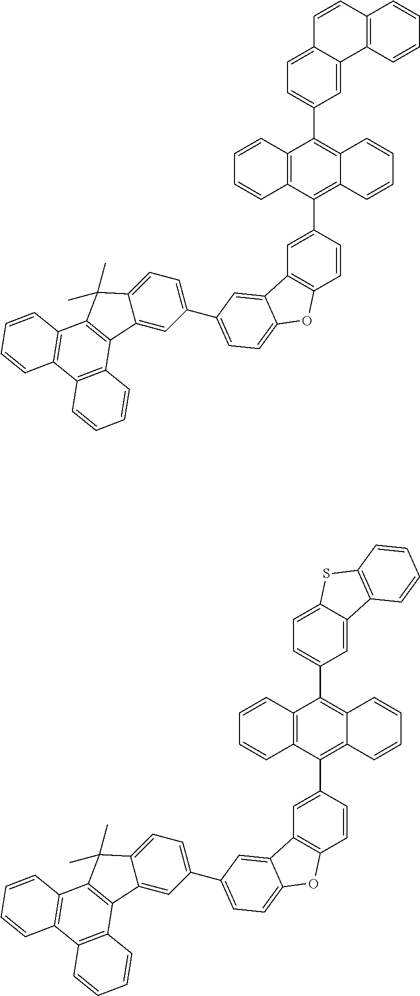 [SMILES: CC1(C)c2ccc(-c3ccc4oc5ccc(-c6c7ccccc7c(-c7ccc8ccc9ccccc9c8c7)c7ccccc67)cc5c4c3)cc2-c2c1c1ccccc1c1ccccc21 ; CC1(C)c2ccc(-c3ccc4oc5ccc(-c6c7ccccc7c(-c7ccc8sc9ccccc9c8c7)c7ccccc67)cc5c4c3)cc2-c2c1c1ccccc1c1ccccc21]

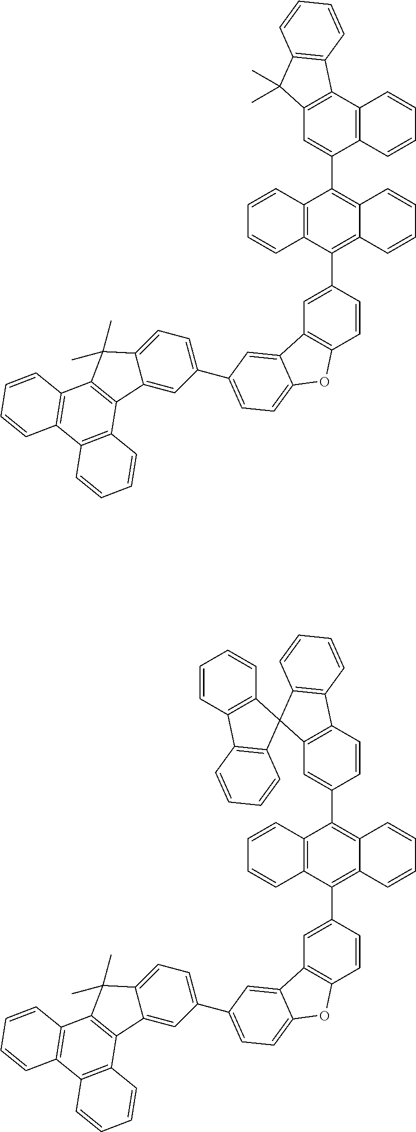 [SMILES: CC1(C)c2ccccc2-c2c1cc(-c1c3ccccc3c(-c3ccc4oc5ccc(-c6ccc7c(c6)-c6c(c8ccccc8c8ccccc68)C7(C)C)cc5c4c3)c3ccccc13)c1ccccc21 ; CC1(C)c2ccc(-c3ccc4oc5ccc(-c6c7ccccc7c(-c7ccc8c(c7)C7(c9ccccc9-c9ccccc97)c7ccccc7-8)c7ccccc67)cc5c4c3)cc2-c2c1c1ccccc1c1ccccc21]

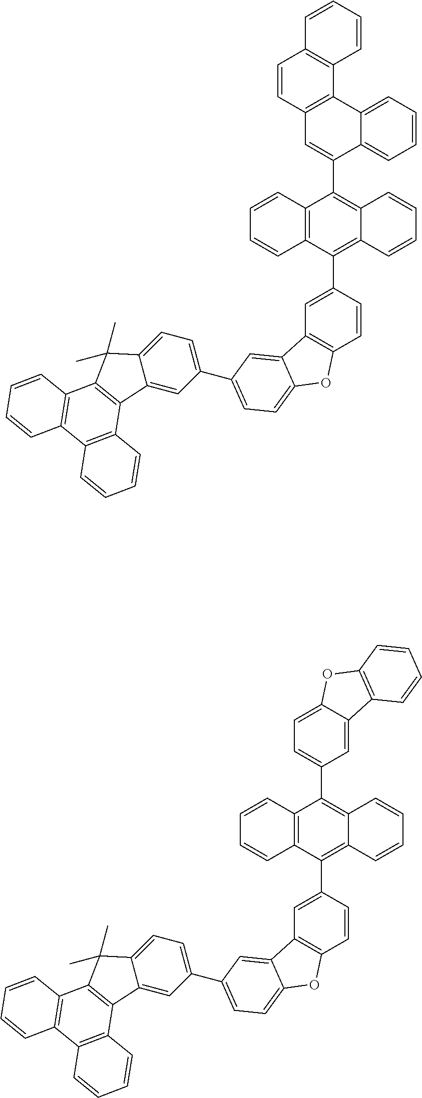 [SMILES: CC1(C)c2ccc(-c3ccc4oc5ccc(-c6c7ccccc7c(-c7cc8ccc9ccccc9c8c8ccccc78)c7ccccc67)cc5c4c3)cc2-c2c1c1ccccc1c1ccccc21 ; CC1(C)c2ccc(-c3ccc4oc5ccc(-c6c7ccccc7c(-c7ccc8oc9ccccc9c8c7)c7ccccc67)cc5c4c3)cc2-c2c1c1ccccc1c1ccccc21]

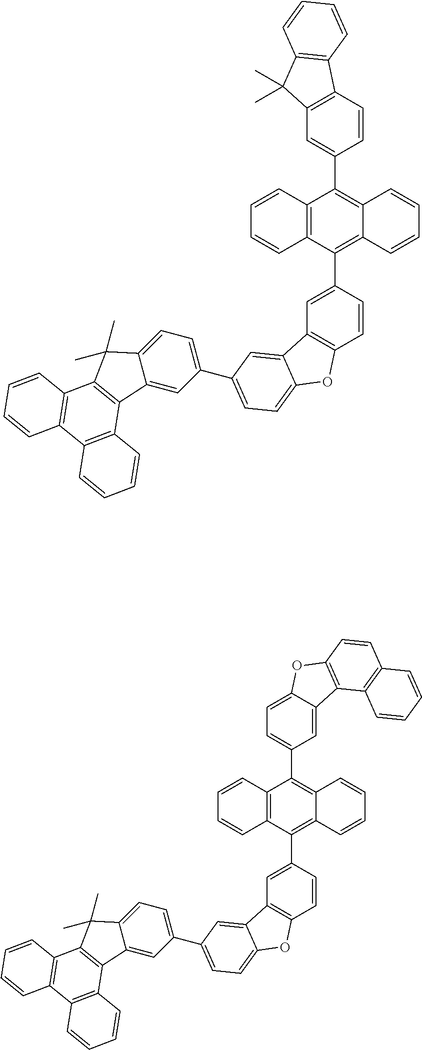 [SMILES: CC1(C)c2ccccc2-c2ccc(-c3c4ccccc4c(-c4ccc5oc6ccc(-c7ccc8c(c7)-c7c(c9ccccc9c9ccccc79)C8(C)C)cc6c5c4)c4ccccc34)cc21 ; CC1(C)c2ccc(-c3ccc4oc5ccc(-c6c7ccccc7c(-c7ccc8oc9ccc%10ccccc%10c9c8c7)c7ccccc67)cc5c4c3)cc2-c2c1c1ccccc1c1ccccc21]

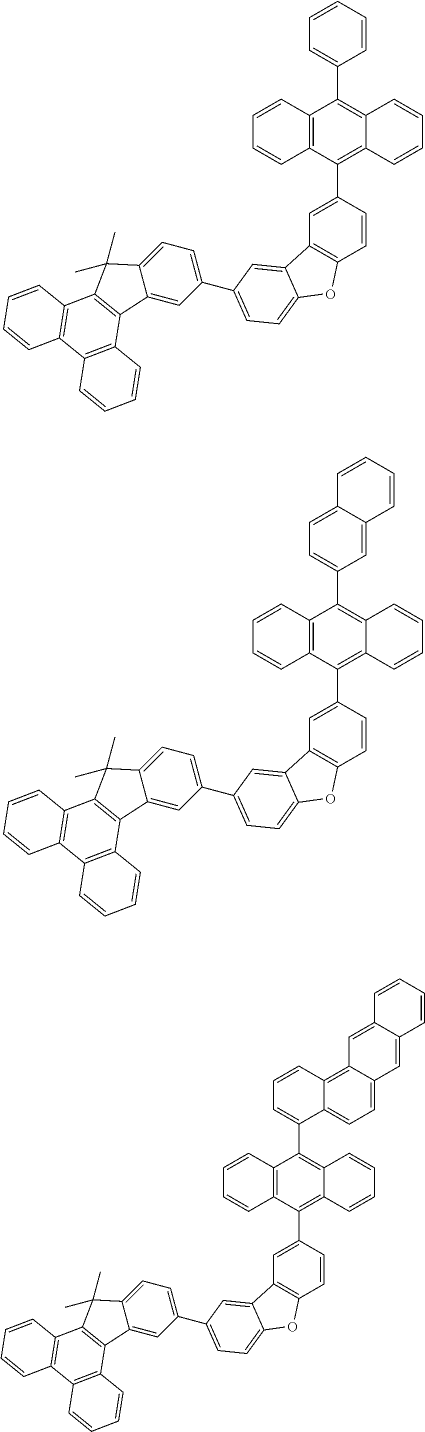 [SMILES: CC1(C)c2ccc(-c3ccc4oc5ccc(-c6c7ccccc7c(-c7ccccc7)c7ccccc67)cc5c4c3)cc2-c2c1c1ccccc1c1ccccc21 ; CC1(C)c2ccc(-c3ccc4oc5ccc(-c6c7ccccc7c(-c7ccc8ccccc8c7)c7ccccc67)cc5c4c3)cc2-c2c1c1ccccc1c1ccccc21 ; CC1(C)c2ccc(-c3ccc4oc5ccc(-c6c7ccccc7c(-c7cccc8c7ccc7cc9ccccc9cc78)c7ccccc67)cc5c4c3)cc2-c2c1c1ccccc1c1ccccc21]

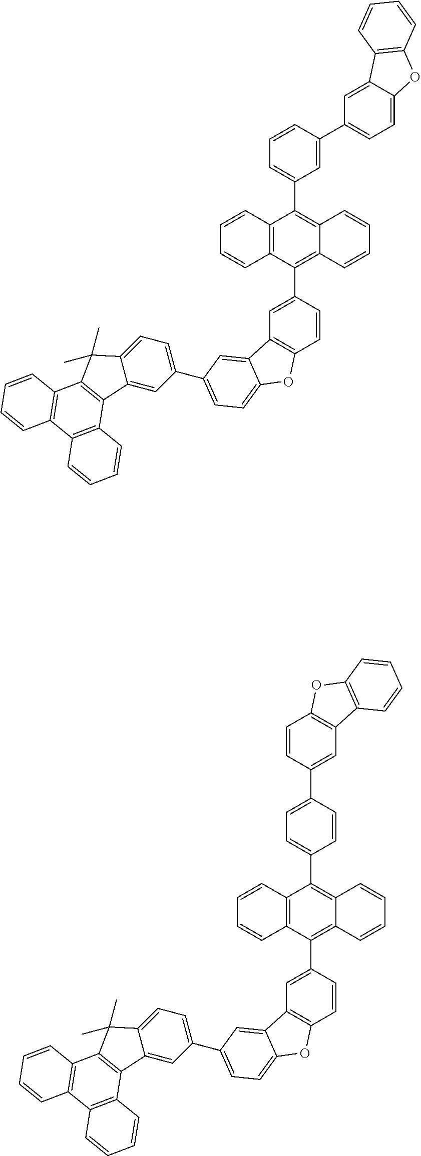 [SMILES: CC1(C)c2ccc(-c3ccc4oc5ccc(-c6c7ccccc7c(-c7cccc(-c8ccc9oc%10ccccc%10c9c8)c7)c7ccccc67)cc5c4c3)cc2-c2c1c1ccccc1c1ccccc21 ; CC1(C)c2ccc(-c3ccc4oc5ccc(-c6c7ccccc7c(-c7ccc(-c8ccc9oc%10ccccc%10c9c8)cc7)c7ccccc67)cc5c4c3)cc2-c2c1c1ccccc1c1ccccc21]

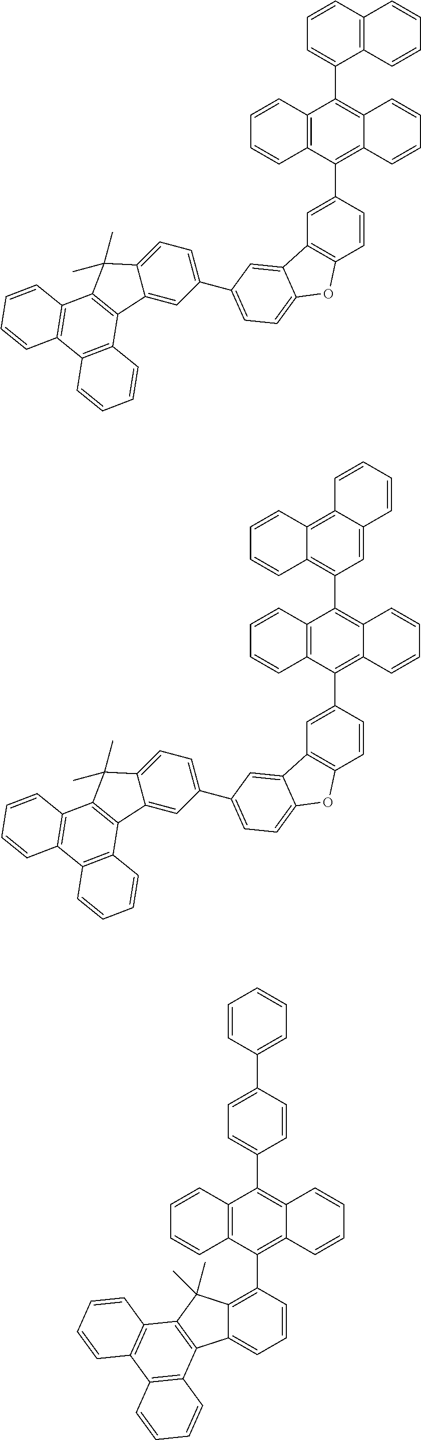

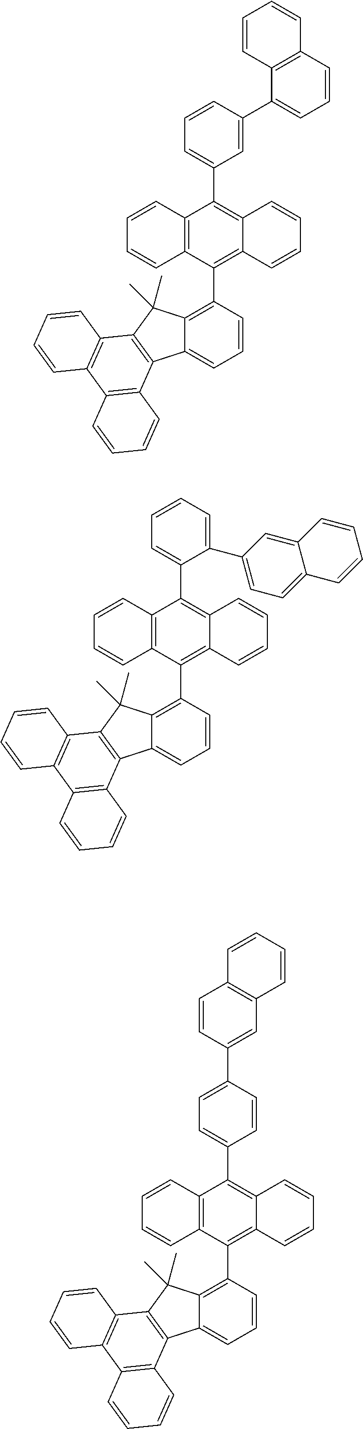

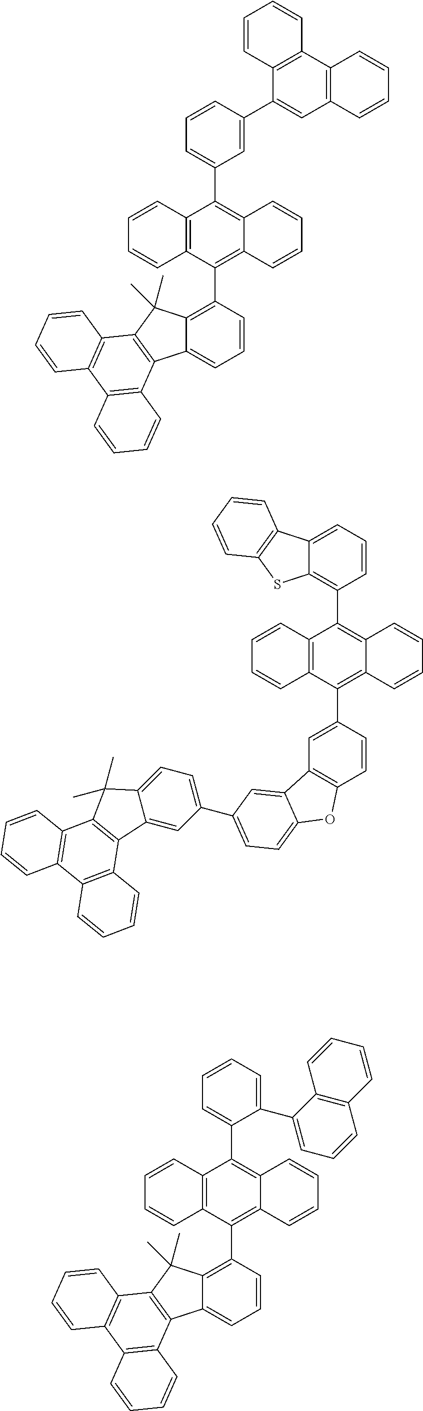

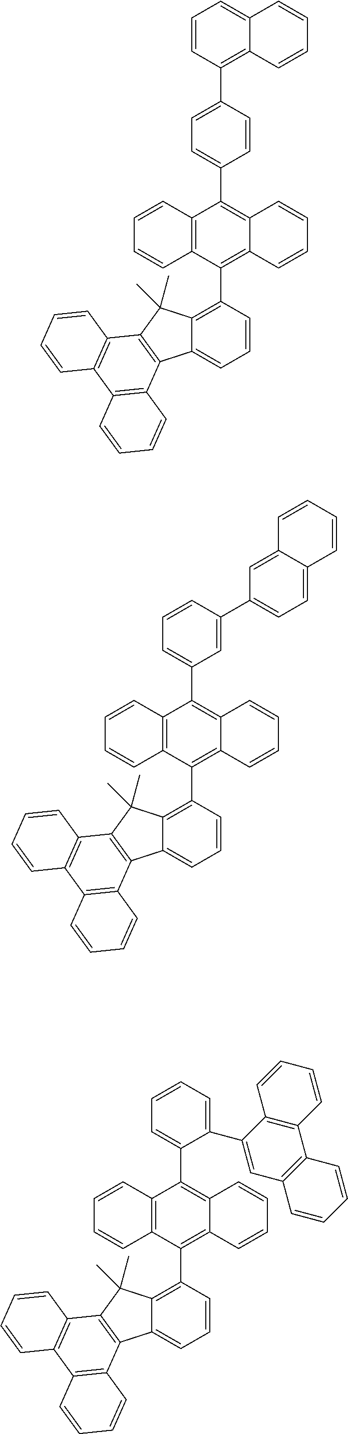

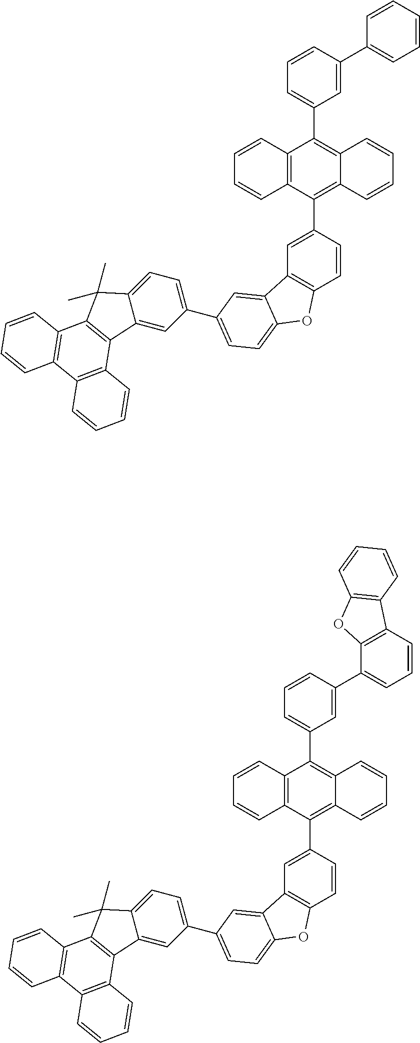 [SMILES: CC1(C)c2ccc(-c3ccc4oc5ccc(-c6c7ccccc7c(-c7cccc(-c8ccccc8)c7)c7ccccc67)cc5c4c3)cc2-c2c1c1ccccc1c1ccccc21 ; CC1(C)c2ccc(-c3ccc4oc5ccc(-c6c7ccccc7c(-c7cccc(-c8cccc9c8oc8ccccc89)c7)c7ccccc67)cc5c4c3)cc2-c2c1c1ccccc1c1ccccc21]

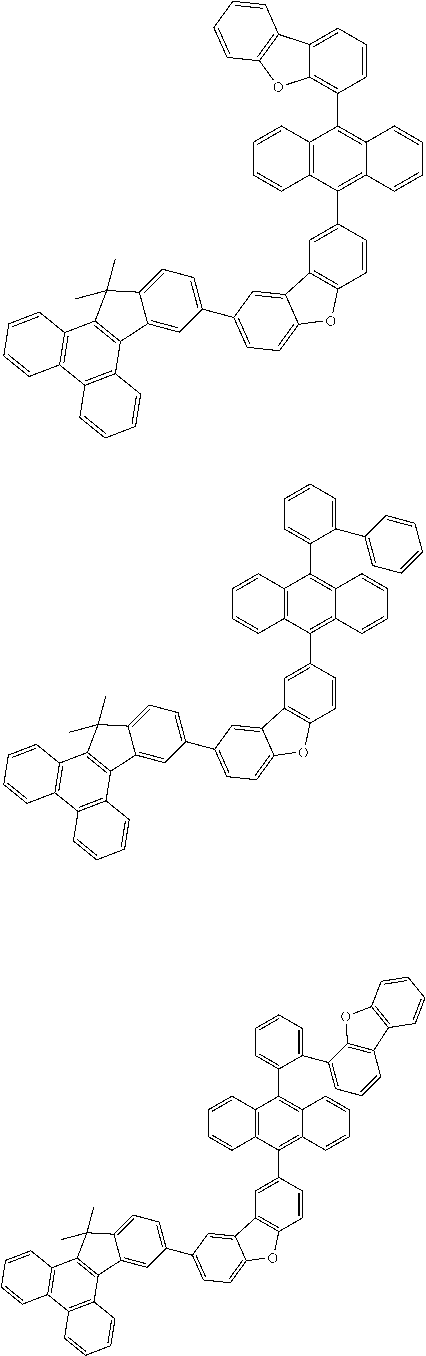 [SMILES: CC1(C)c2ccc(-c3ccc4oc5ccc(-c6c7ccccc7c(-c7cccc8c7oc7ccccc78)c7ccccc67)cc5c4c3)cc2-c2c1c1ccccc1c1ccccc21 ; CC1(C)c2ccc(-c3ccc4oc5ccc(-c6c7ccccc7c(-c7ccccc7-c7ccccc7)c7ccccc67)cc5c4c3)cc2-c2c1c1ccccc1c1ccccc21 ; CC1(C)c2ccc(-c3ccc4oc5ccc(-c6c7ccccc7c(-c7ccccc7-c7cccc8c7oc7ccccc78)c7ccccc67)cc5c4c3)cc2-c2c1c1ccccc1c1ccccc21]

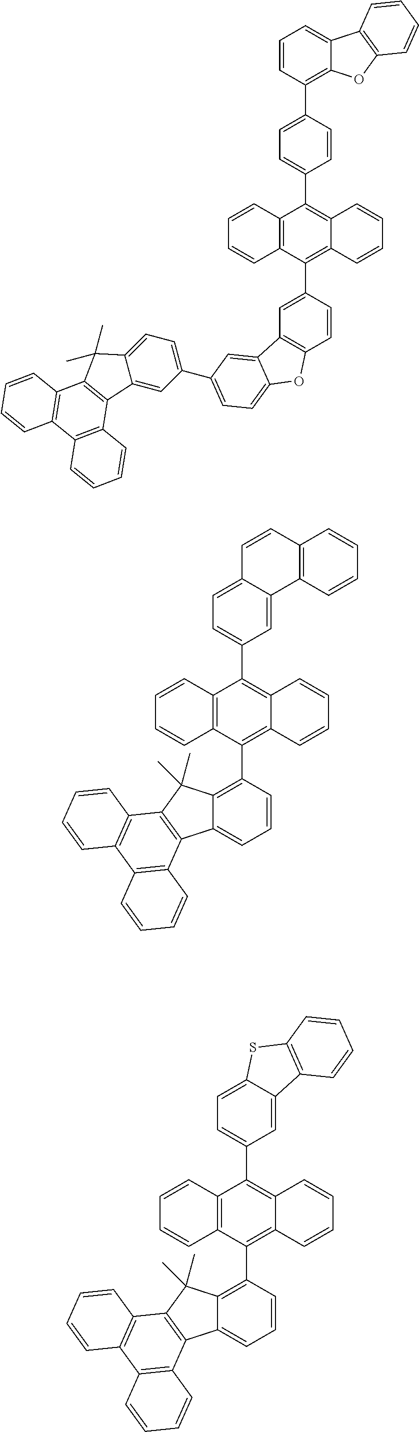

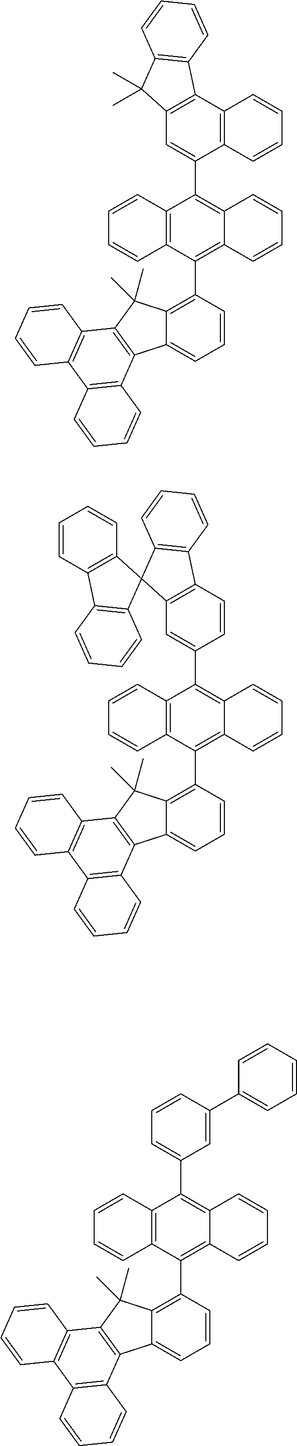

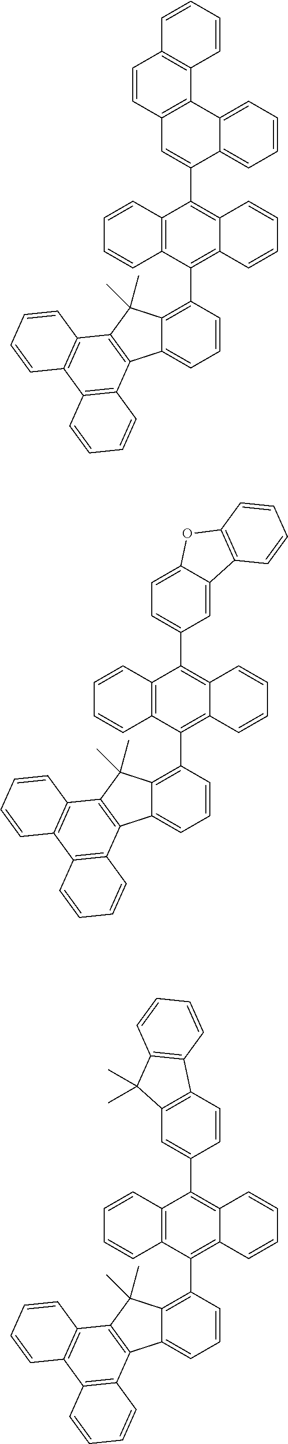

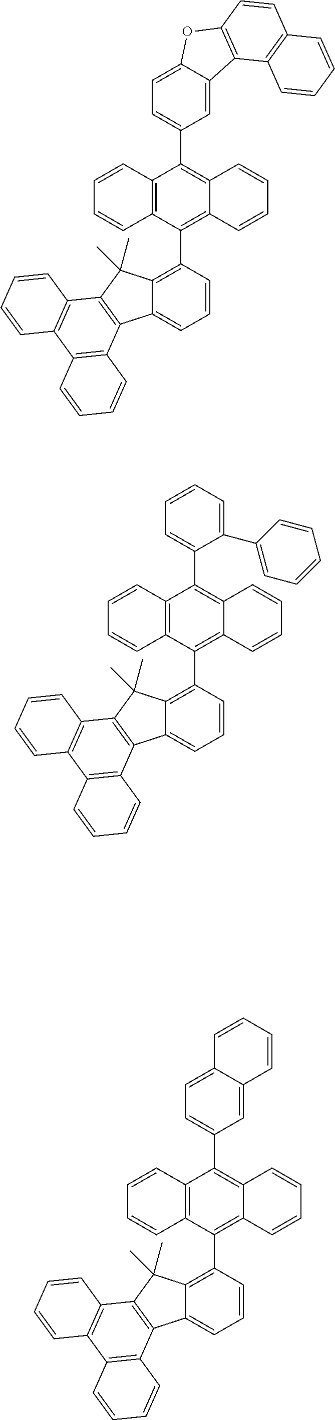

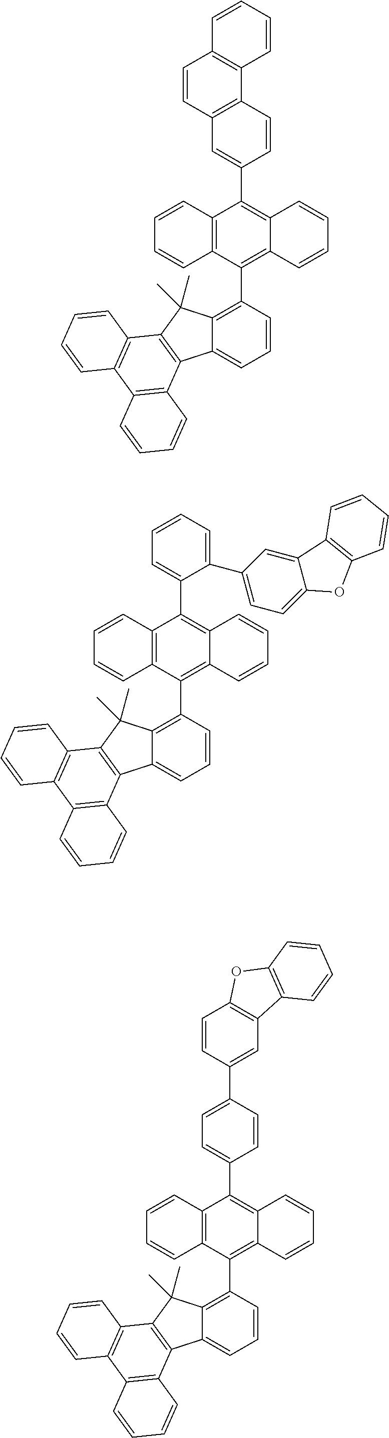

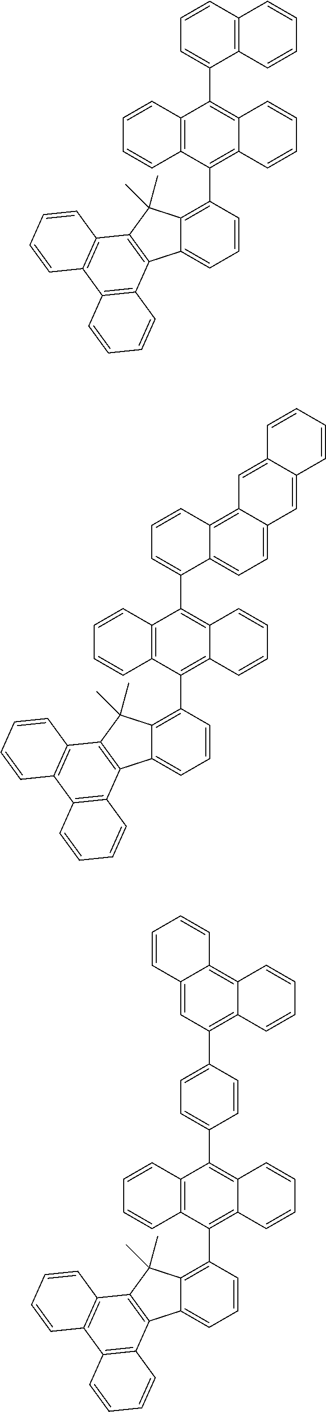

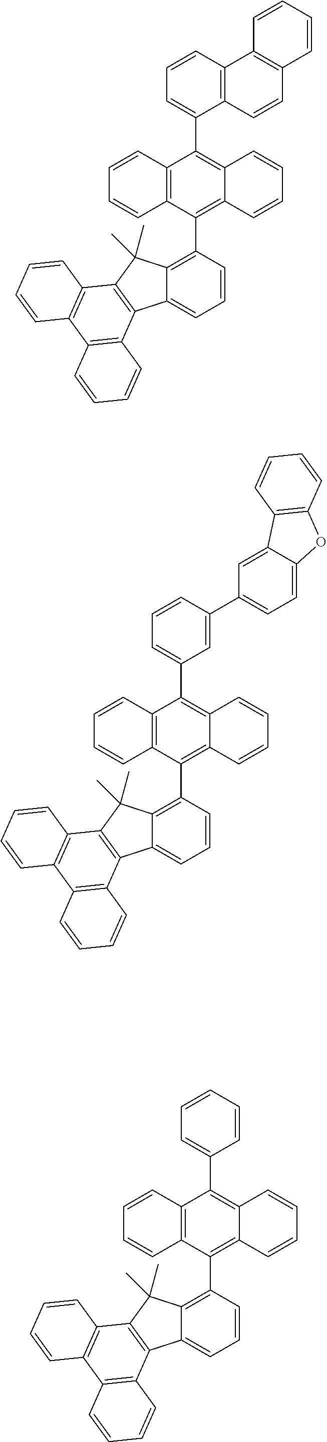

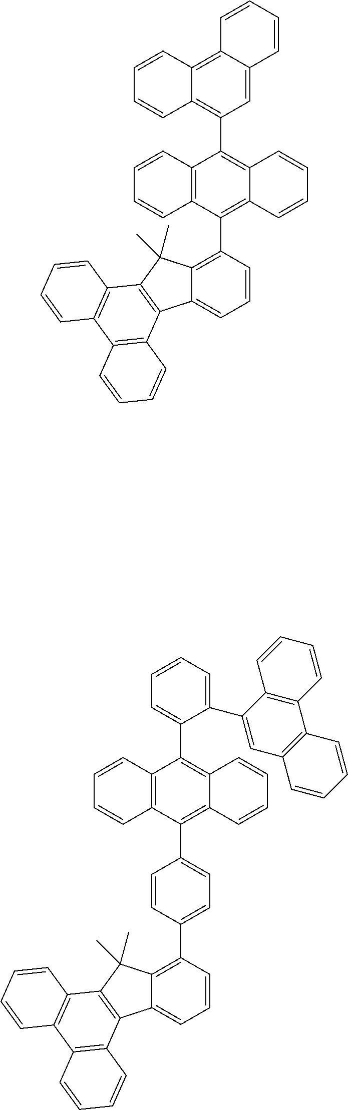

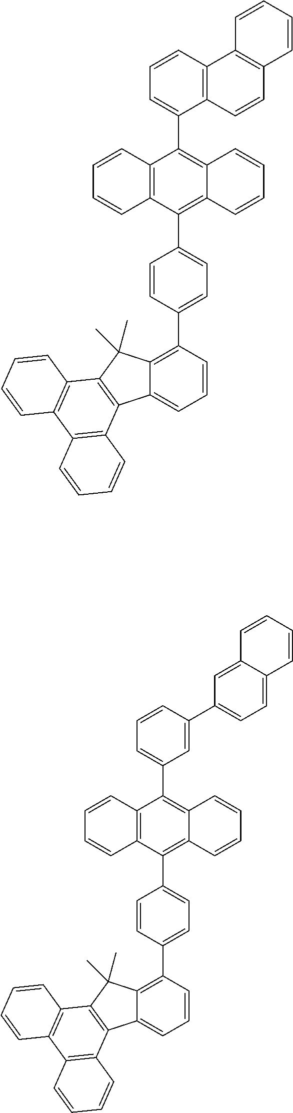

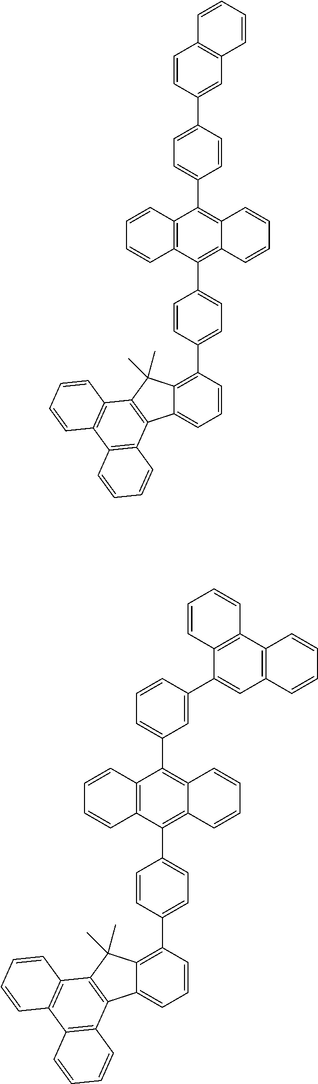

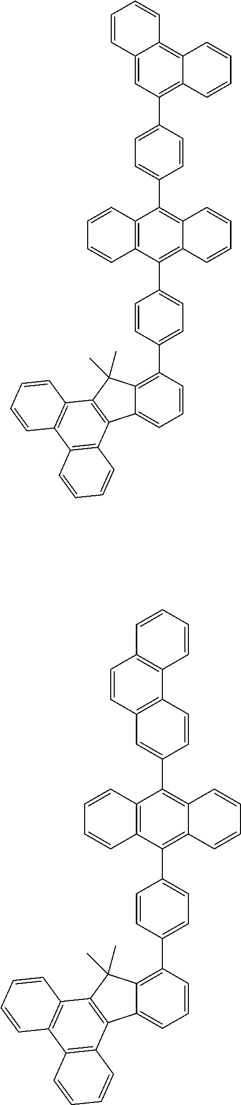

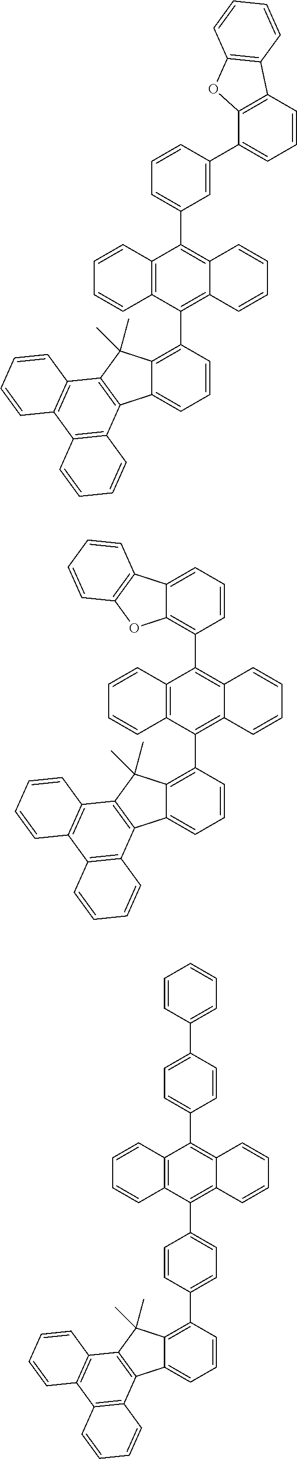

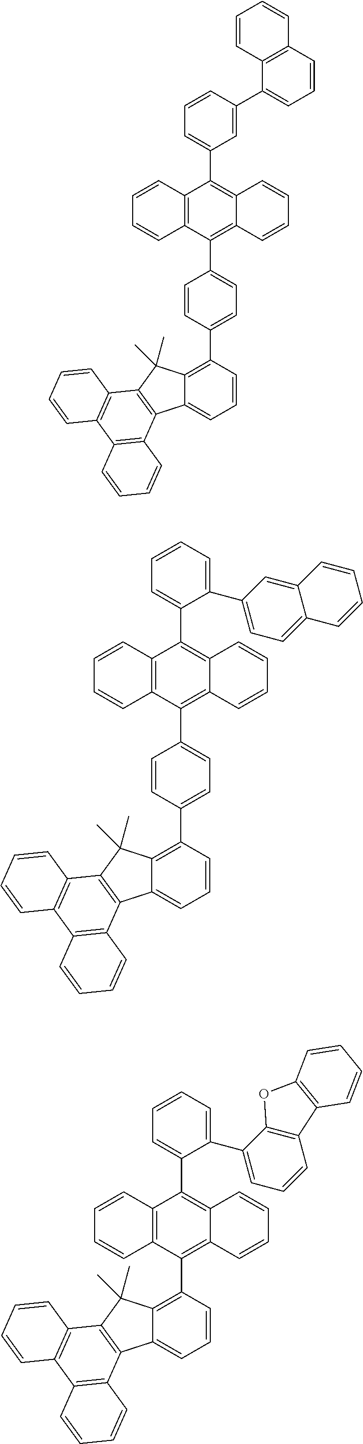

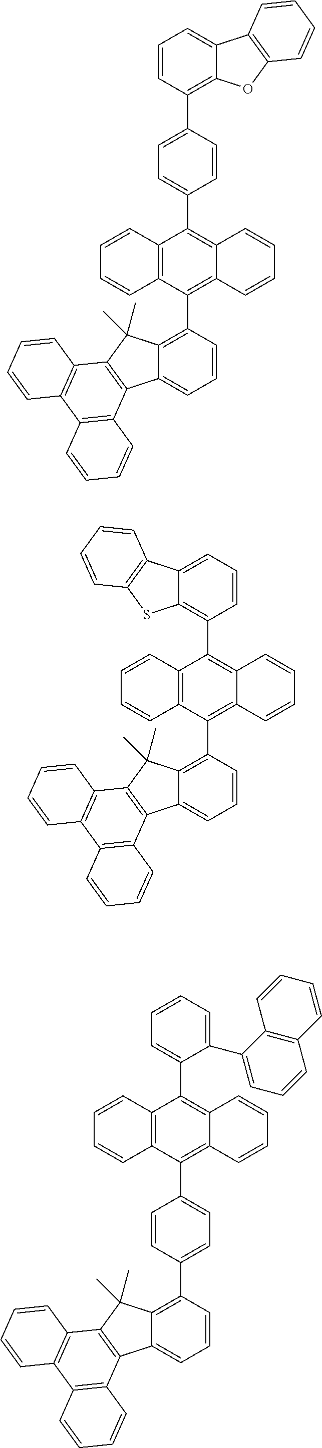

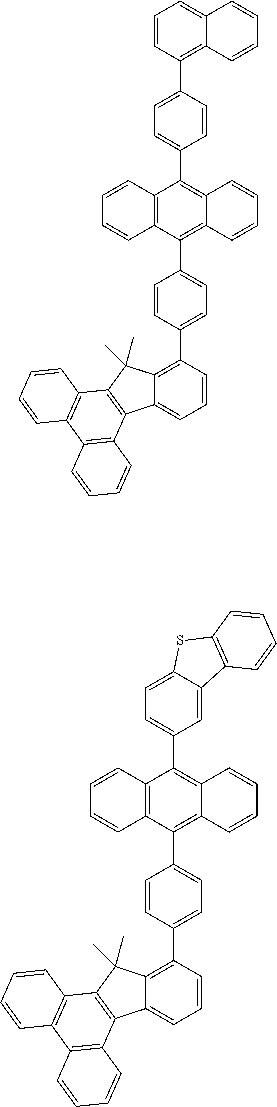

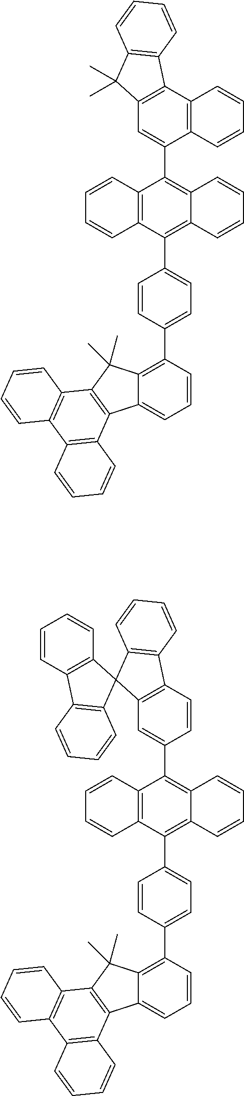

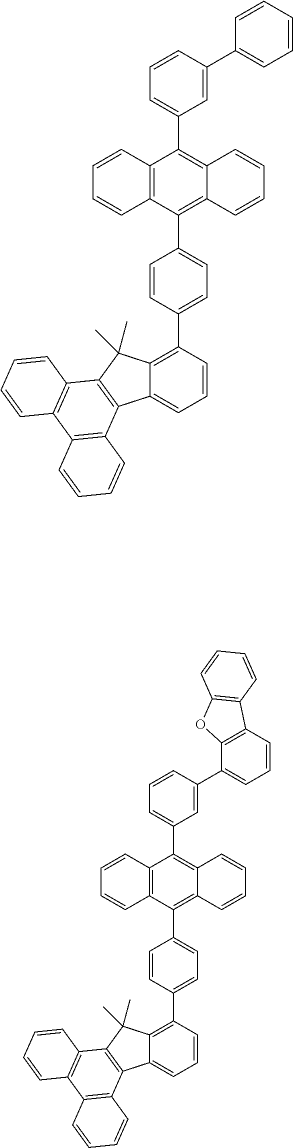

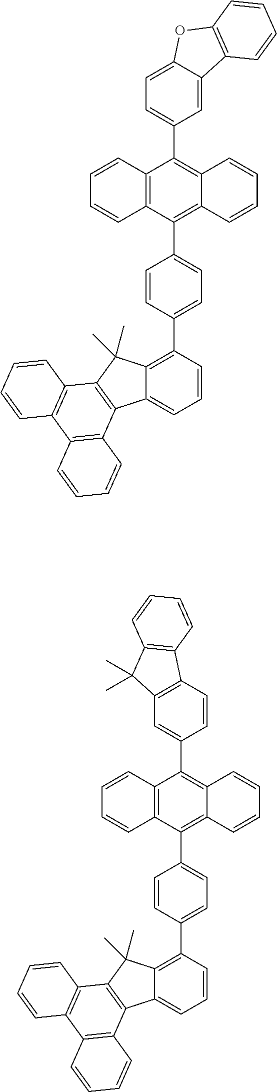

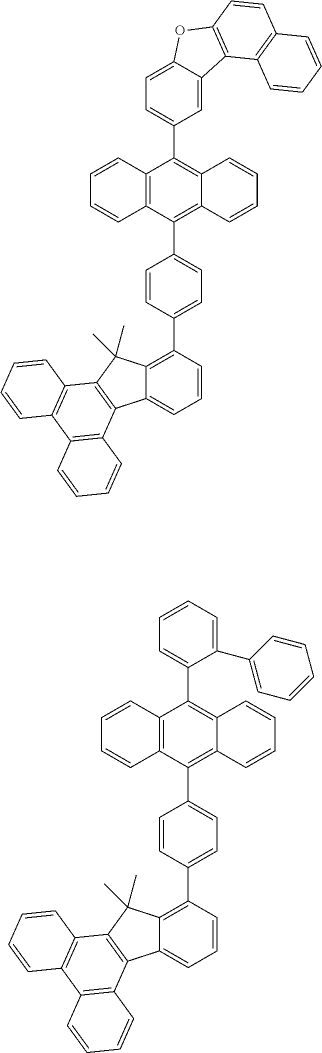

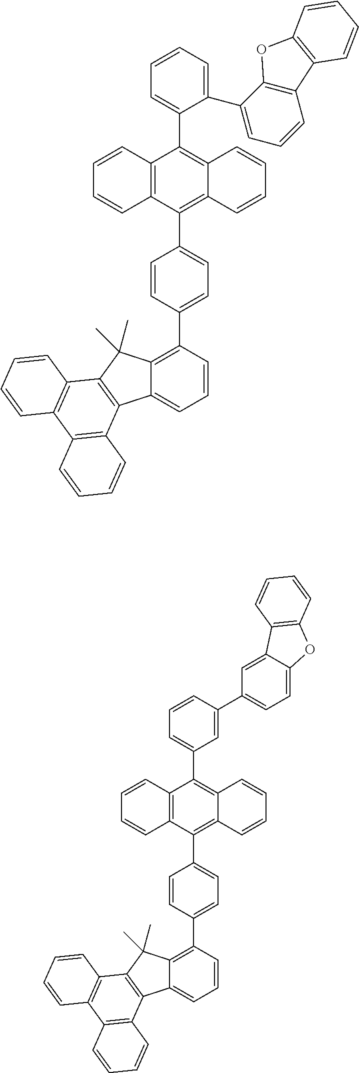 [SMILES: CC1(C)c2c(-c3ccc(-c4c5ccccc5c(-c5ccccc5-c5cccc6c5oc5ccccc56)c5ccccc45)cc3)cccc2-c2c1c1ccccc1c1ccccc21 ; CC1(C)c2c(-c3ccc(-c4c5ccccc5c(-c5cccc(-c6ccc7oc8ccccc8c7c6)c5)c5ccccc45)cc3)cccc2-c2c1c1ccccc1c1ccccc21]

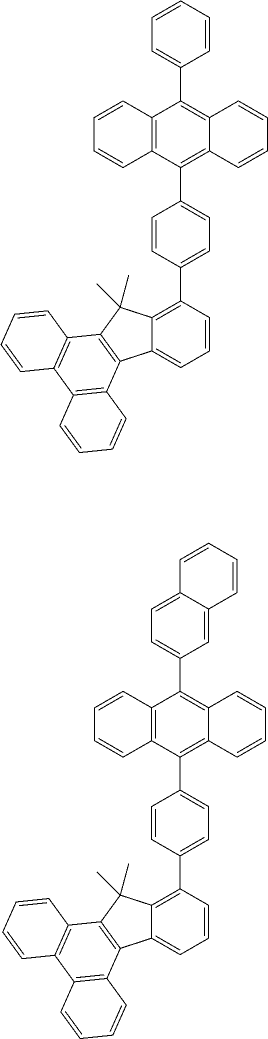

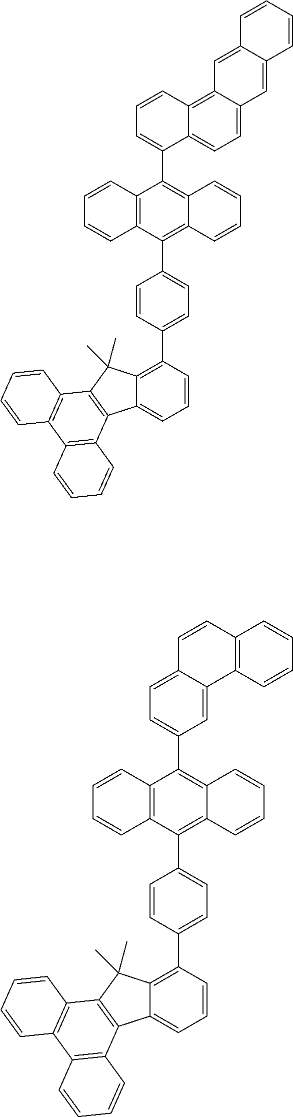

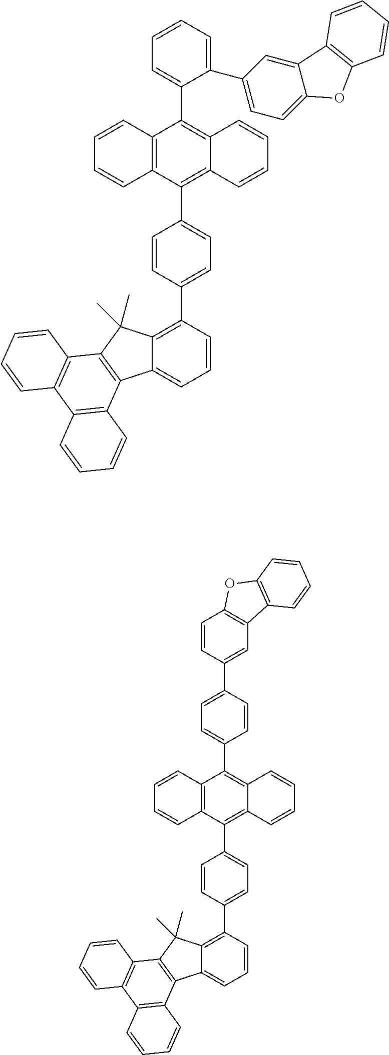 [SMILES: CC1(C)c2c(-c3ccc(-c4c5ccccc5c(-c5ccccc5-c5ccc6oc7ccccc7c6c5)c5ccccc45)cc3)cccc2-c2c1c1ccccc1c1ccccc21 ; CC1(C)c2c(-c3ccc(-c4c5ccccc5c(-c5ccc(-c6ccc7oc8ccccc8c7c6)cc5)c5ccccc45)cc3)cccc2-c2c1c1ccccc1c1ccccc21]

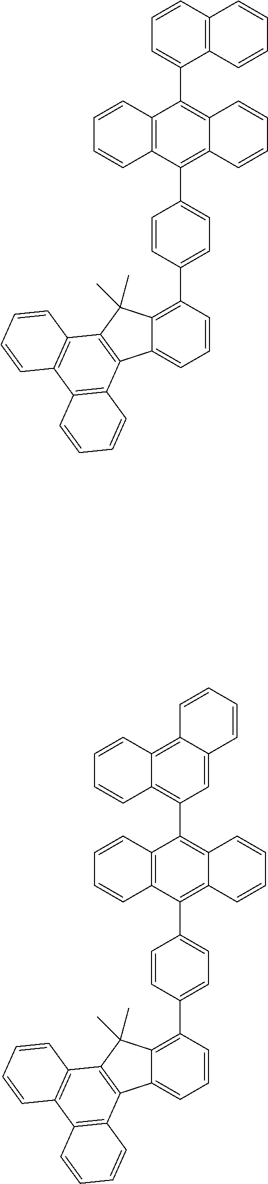

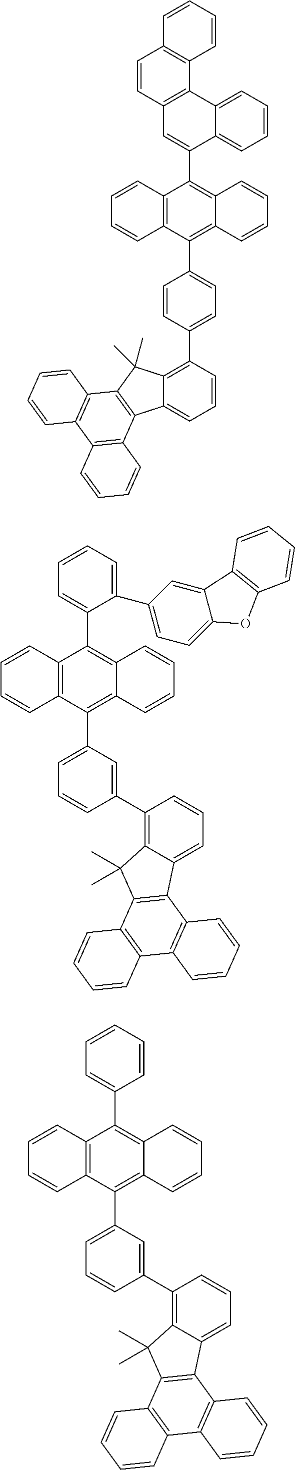

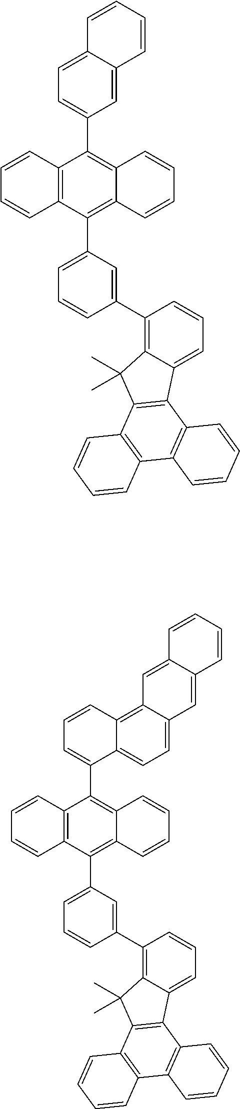

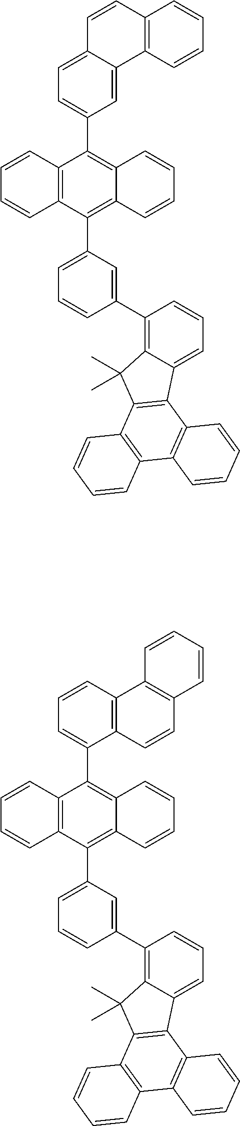

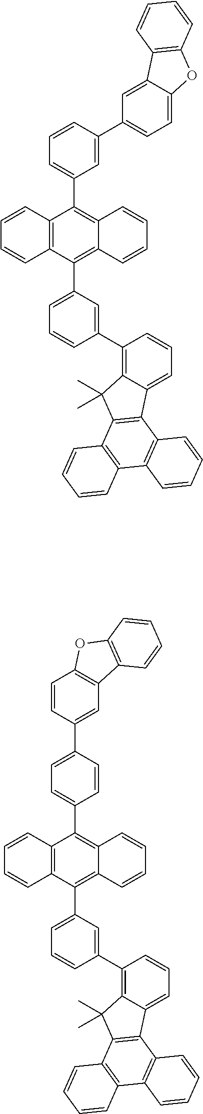 [SMILES: CC1(C)c2c(-c3cccc(-c4c5ccccc5c(-c5cccc(-c6ccc7oc8ccccc8c7c6)c5)c5ccccc45)c3)cccc2-c2c1c1ccccc1c1ccccc21 ; CC1(C)c2c(-c3cccc(-c4c5ccccc5c(-c5ccc(-c6ccc7oc8ccccc8c7c6)cc5)c5ccccc45)c3)cccc2-c2c1c1ccccc1c1ccccc21]

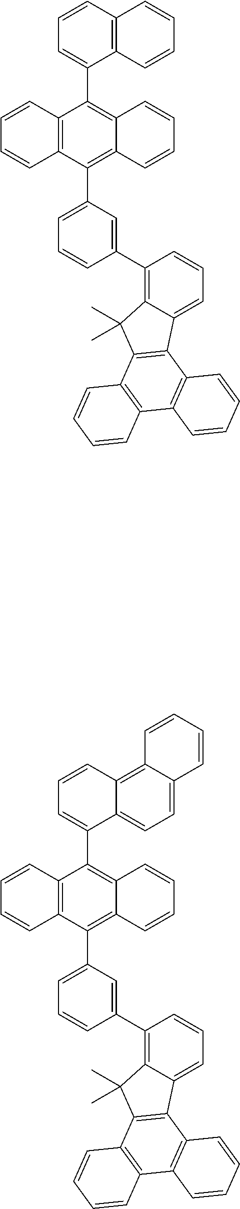

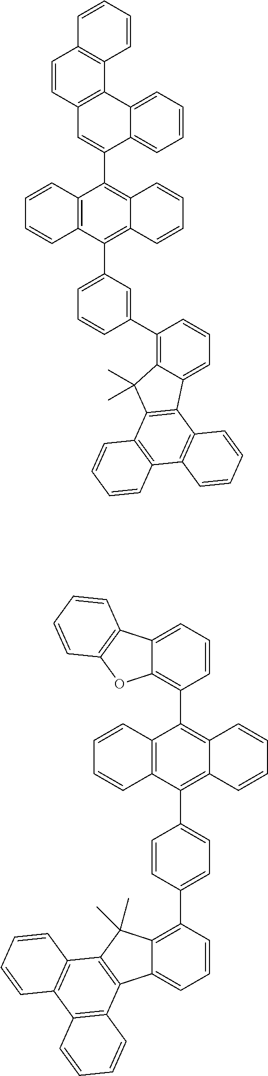

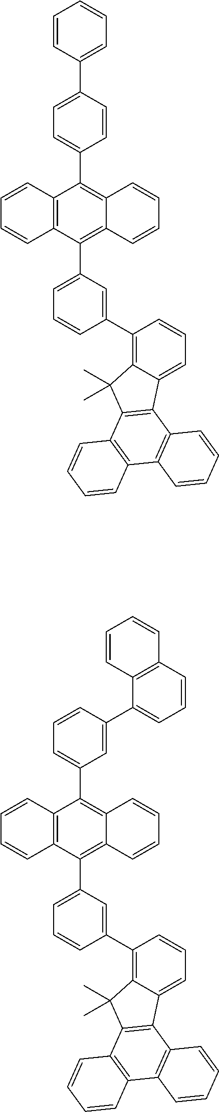

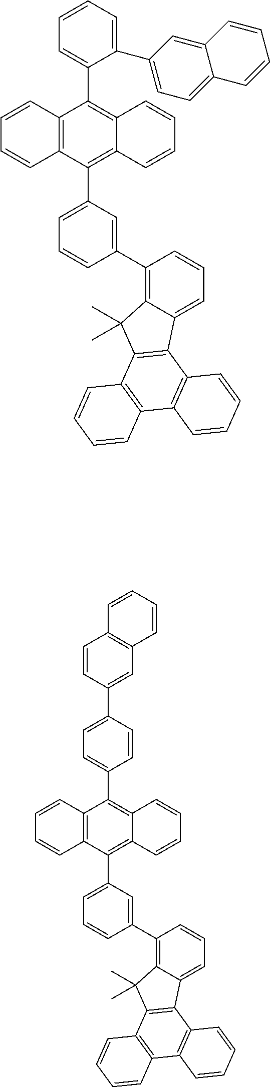

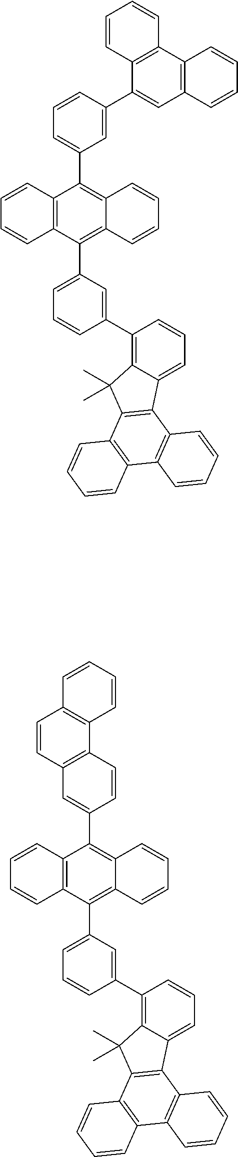

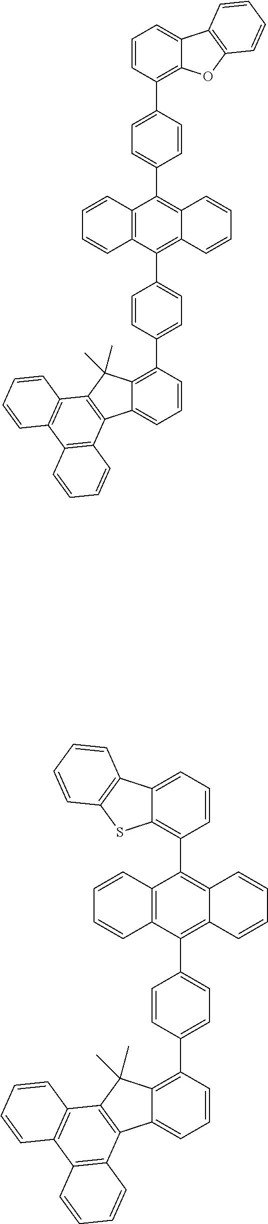 [SMILES: CC1(C)c2c(-c3ccc(-c4c5ccccc5c(-c5ccc(-c6cccc7c6oc6ccccc67)cc5)c5ccccc45)cc3)cccc2-c2c1c1ccccc1c1ccccc21 ; CC1(C)c2c(-c3ccc(-c4c5ccccc5c(-c5cccc6c5sc5ccccc56)c5ccccc45)cc3)cccc2-c2c1c1ccccc1c1ccccc21]

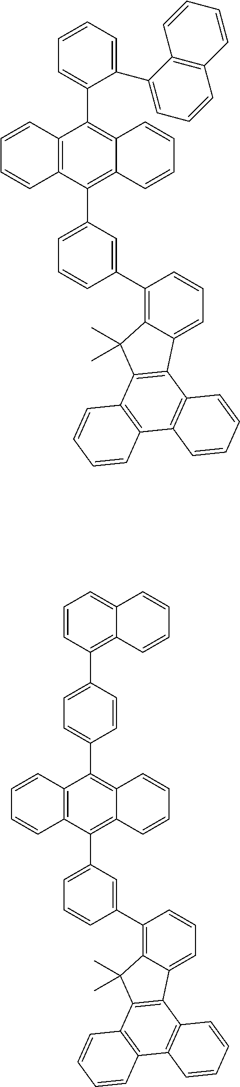

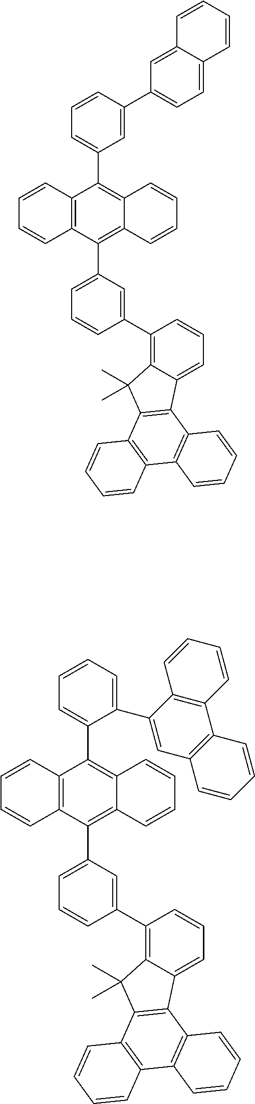

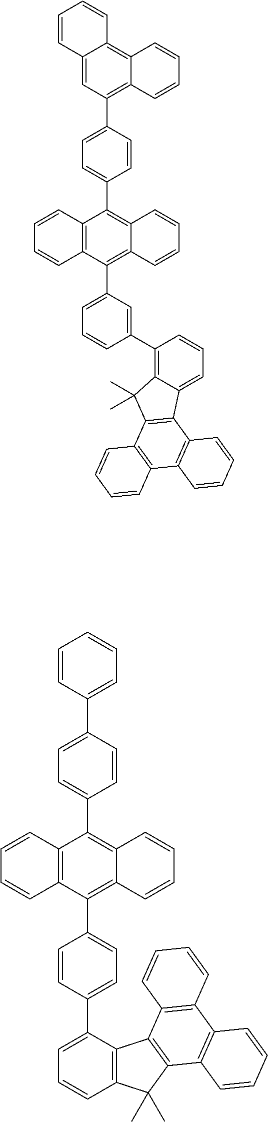

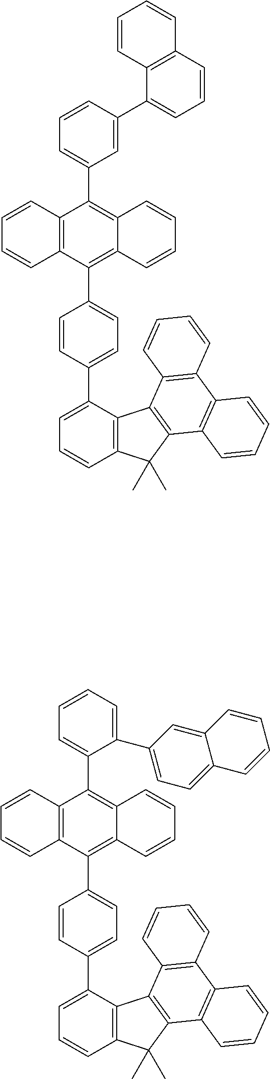

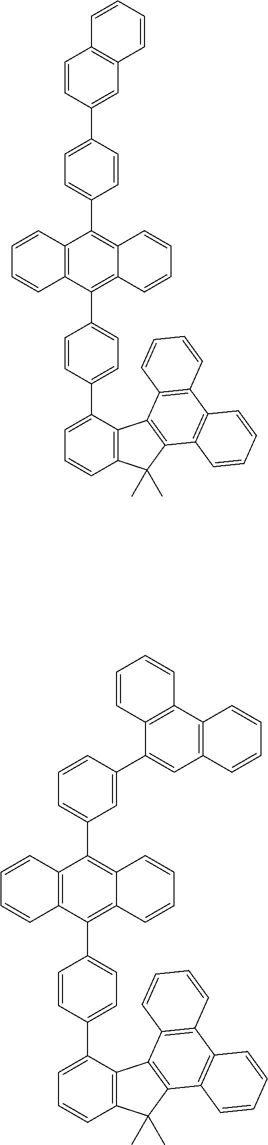

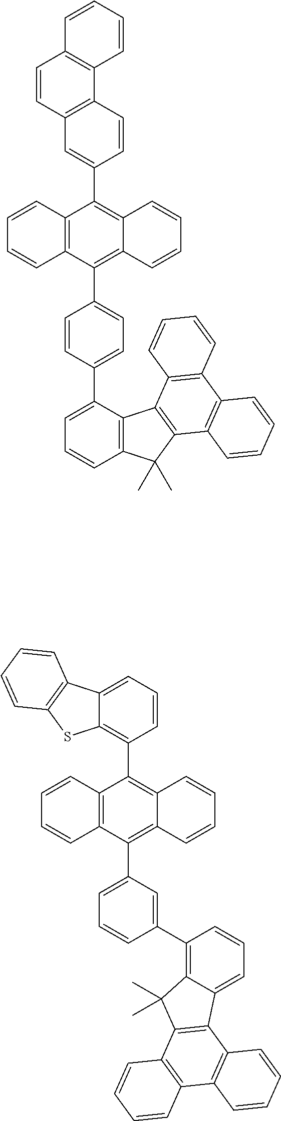

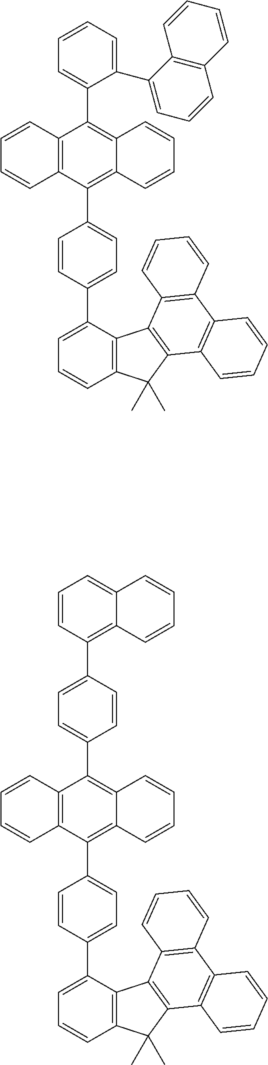

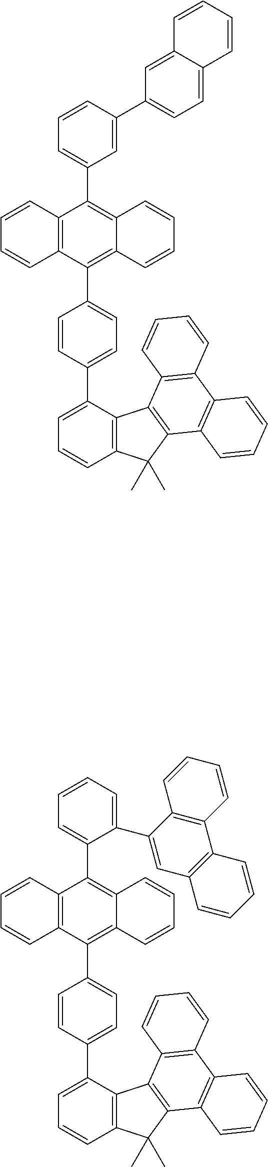

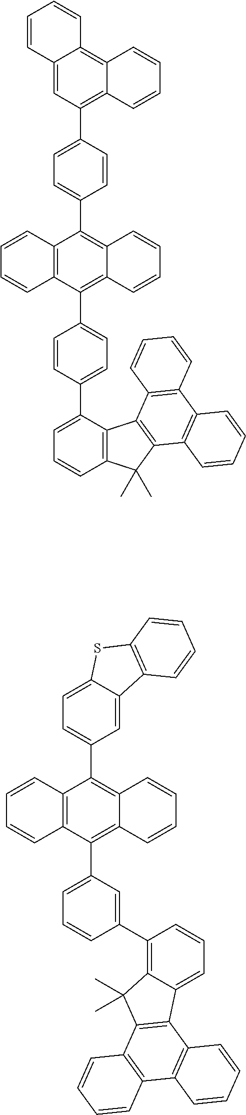

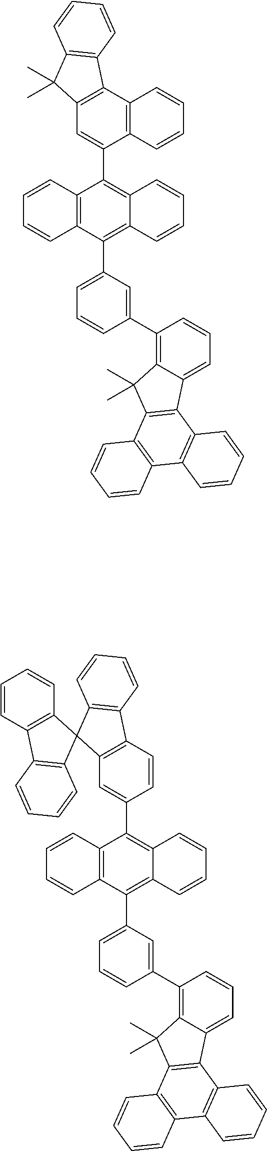

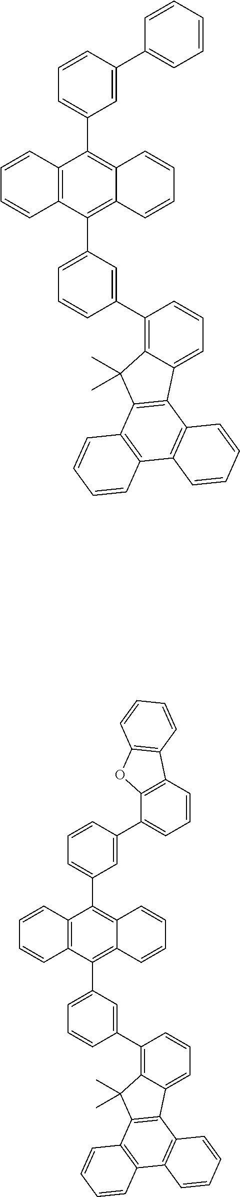

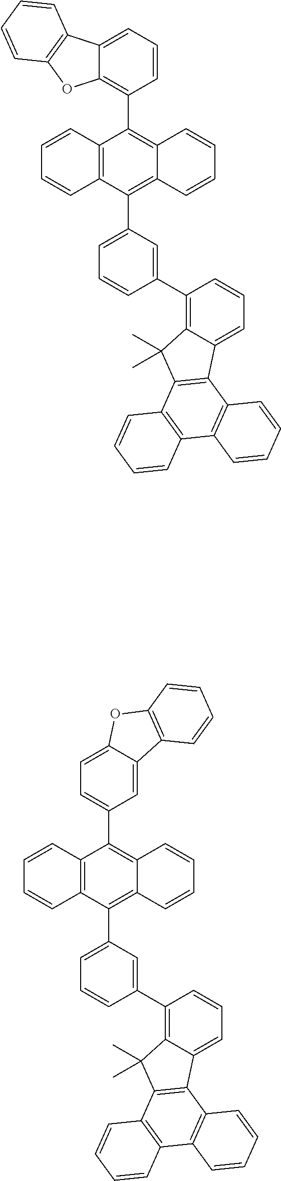 [SMILES: CC1(C)c2c(-c3cccc(-c4c5ccccc5c(-c5cccc6c5oc5ccccc56)c5ccccc45)c3)cccc2-c2c1c1ccccc1c1ccccc21 ; CC1(C)c2c(-c3cccc(-c4c5ccccc5c(-c5ccc6oc7ccccc7c6c5)c5ccccc45)c3)cccc2-c2c1c1ccccc1c1ccccc21]

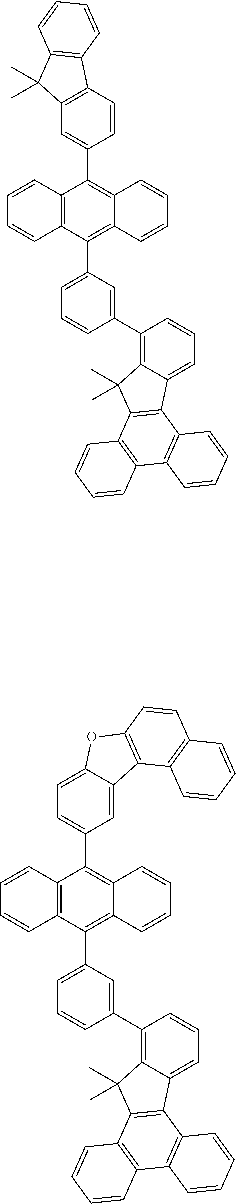

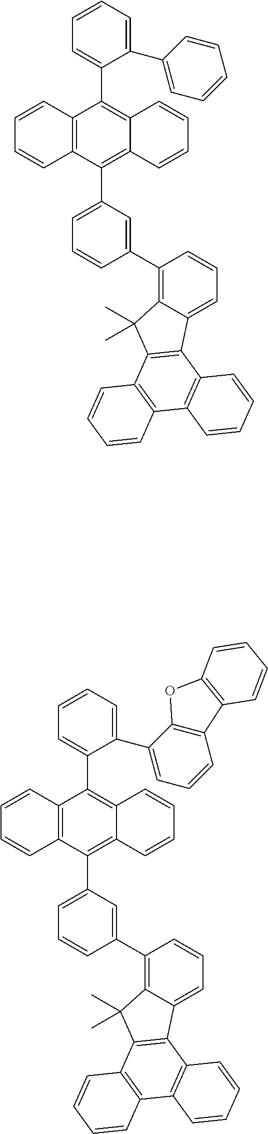

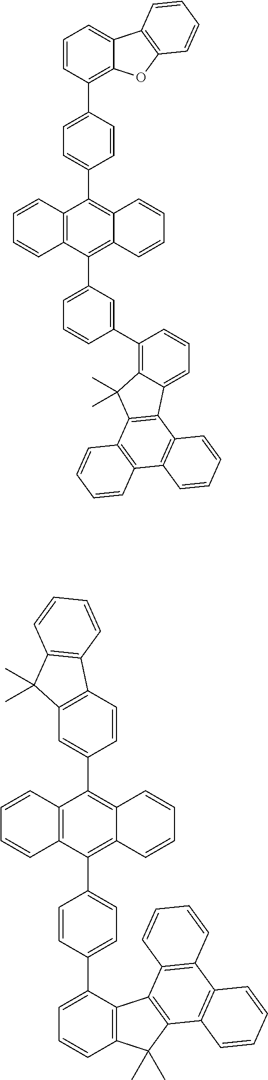

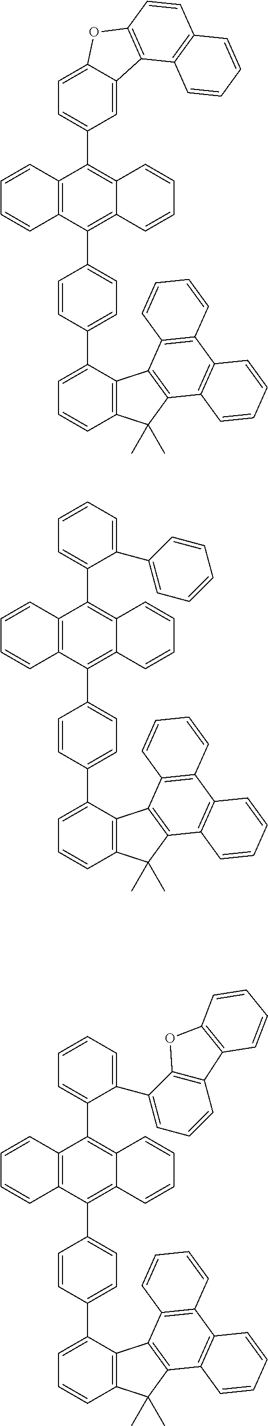

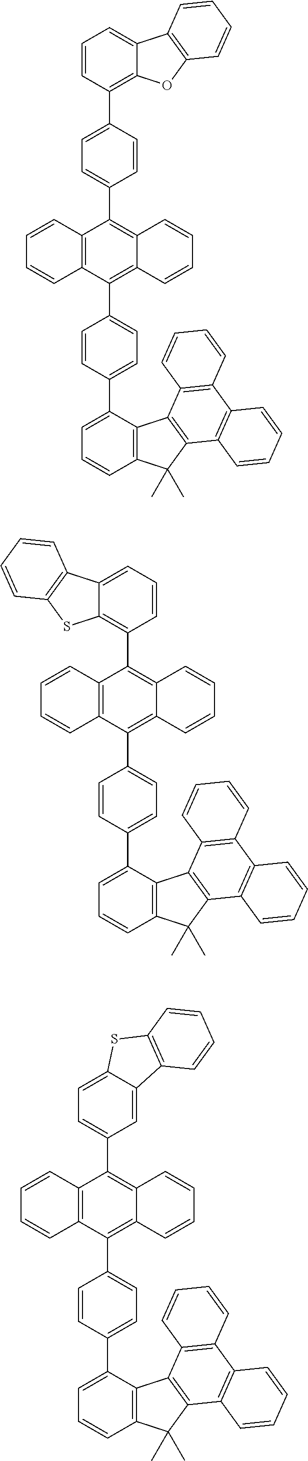 [SMILES: CC1(C)c2cccc(-c3ccc(-c4c5ccccc5c(-c5ccc(-c6cccc7c6oc6ccccc67)cc5)c5ccccc45)cc3)c2-c2c1c1ccccc1c1ccccc21 ; CC1(C)c2cccc(-c3ccc(-c4c5ccccc5c(-c5cccc6c5sc5ccccc56)c5ccccc45)cc3)c2-c2c1c1ccccc1c1ccccc21 ; CC1(C)c2cccc(-c3ccc(-c4c5ccccc5c(-c5ccc6sc7ccccc7c6c5)c5ccccc45)cc3)c2-c2c1c1ccccc1c1ccccc21]

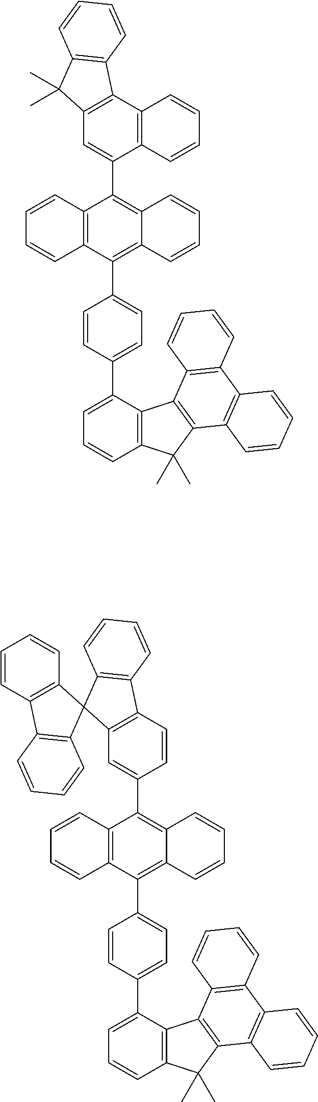

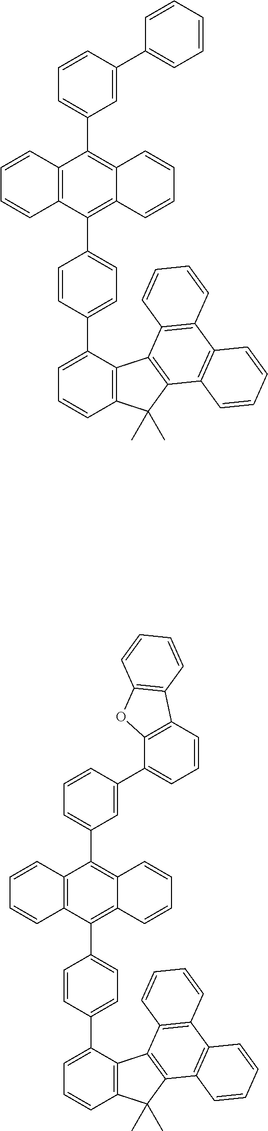

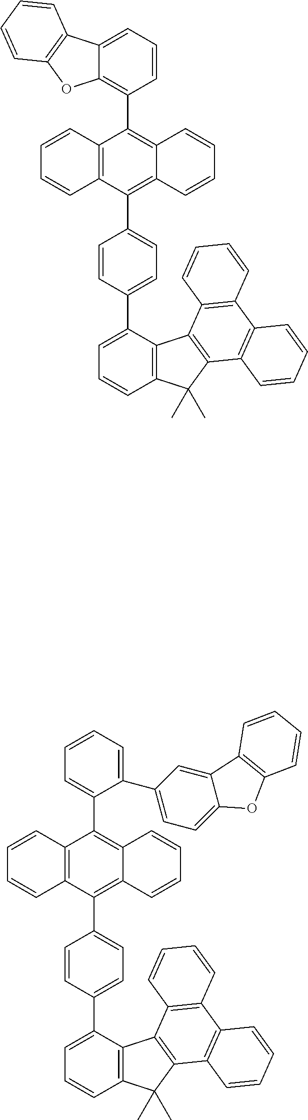 [SMILES: CC1(C)c2cccc(-c3ccc(-c4c5ccccc5c(-c5cccc6c5oc5ccccc56)c5ccccc45)cc3)c2-c2c1c1ccccc1c1ccccc21 ; CC1(C)c2cccc(-c3ccc(-c4c5ccccc5c(-c5ccccc5-c5ccc6oc7ccccc7c6c5)c5ccccc45)cc3)c2-c2c1c1ccccc1c1ccccc21]

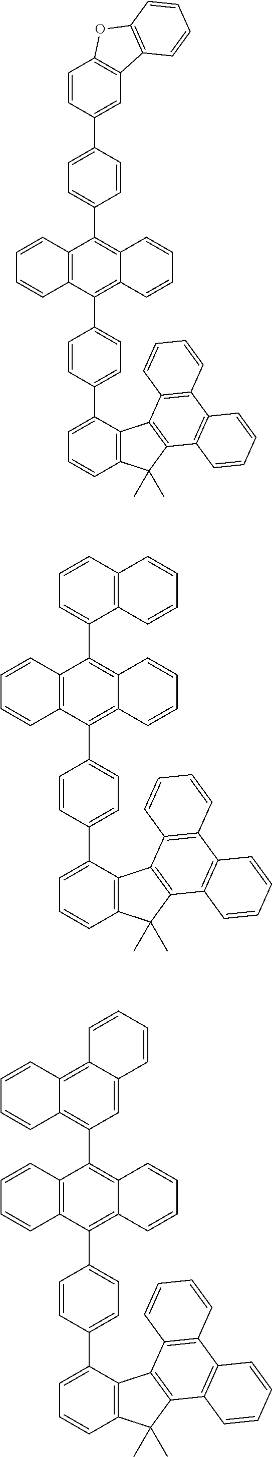

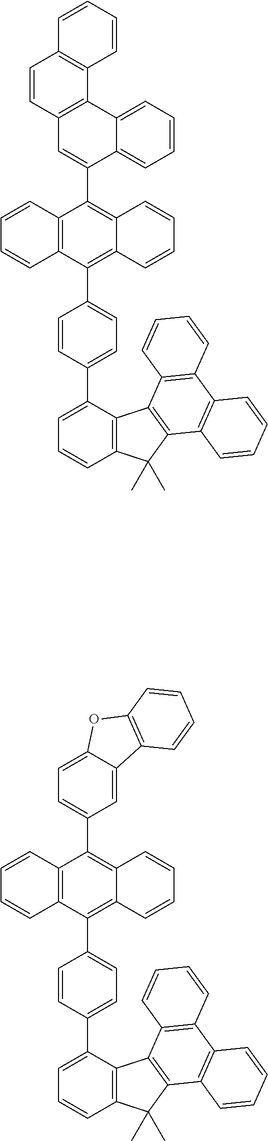

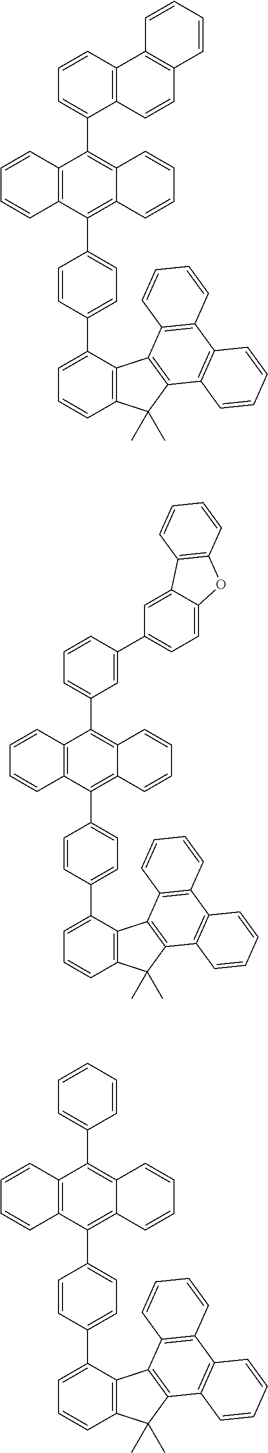

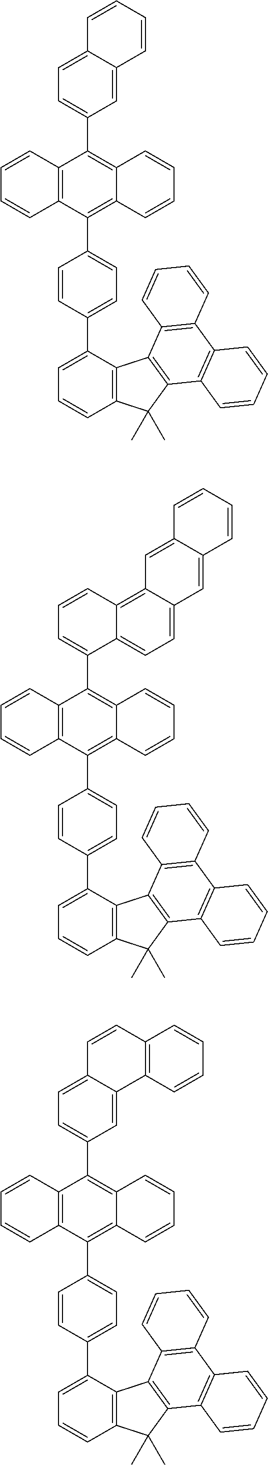

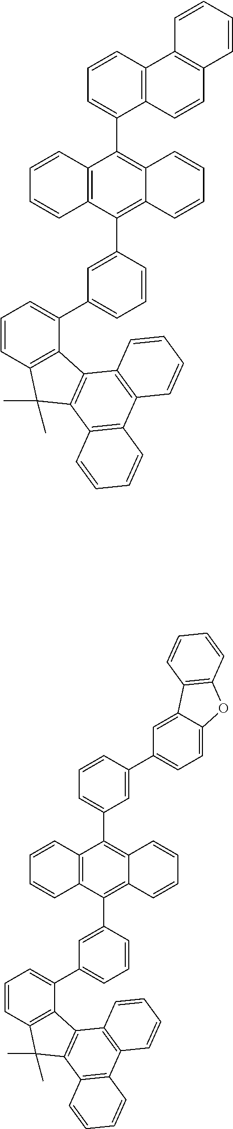

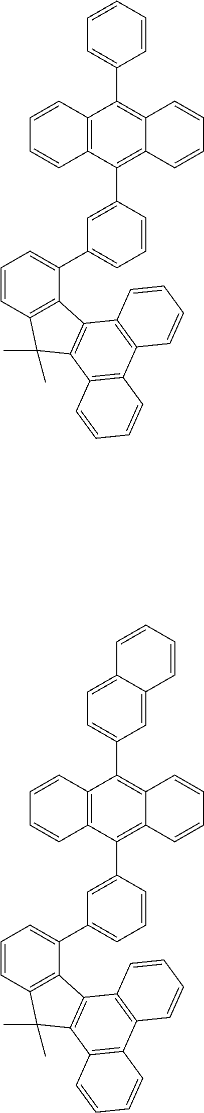

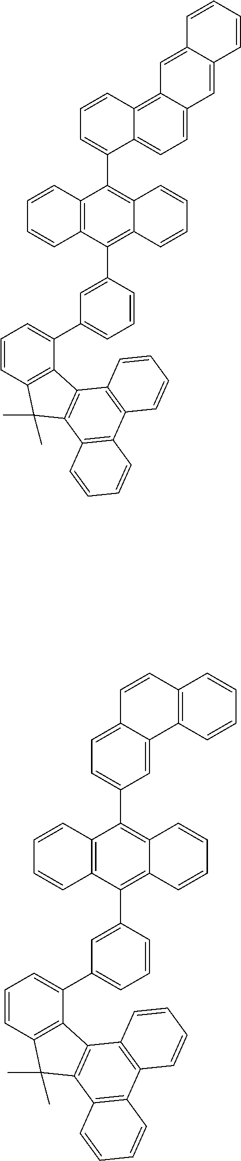

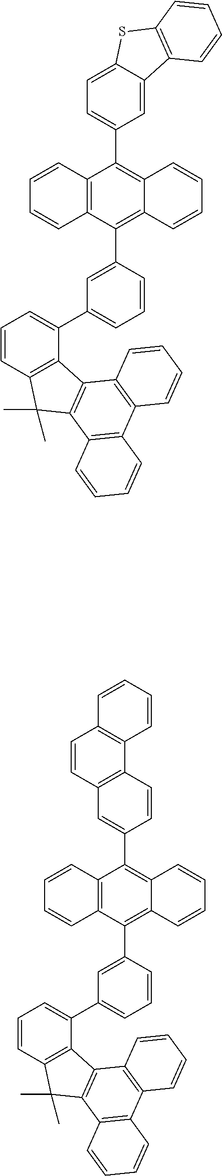

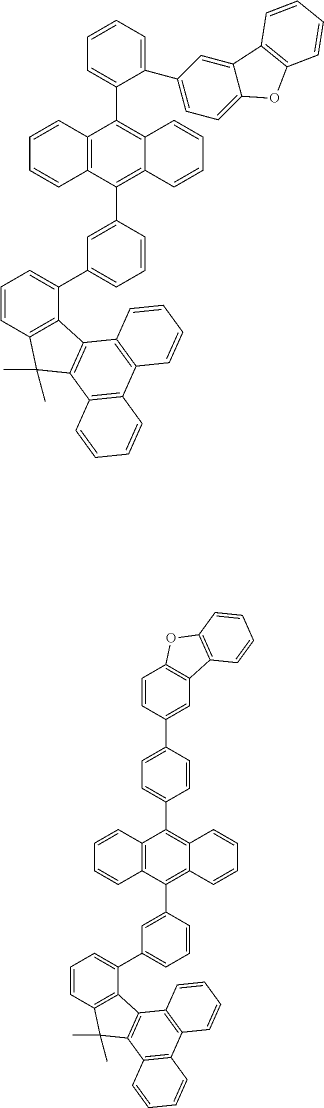 [SMILES: CC1(C)c2cccc(-c3cccc(-c4c5ccccc5c(-c5ccccc5-c5ccc6oc7ccccc7c6c5)c5ccccc45)c3)c2-c2c1c1ccccc1c1ccccc21 ; CC1(C)c2cccc(-c3cccc(-c4c5ccccc5c(-c5ccc(-c6ccc7oc8ccccc8c7c6)cc5)c5ccccc45)c3)c2-c2c1c1ccccc1c1ccccc21]

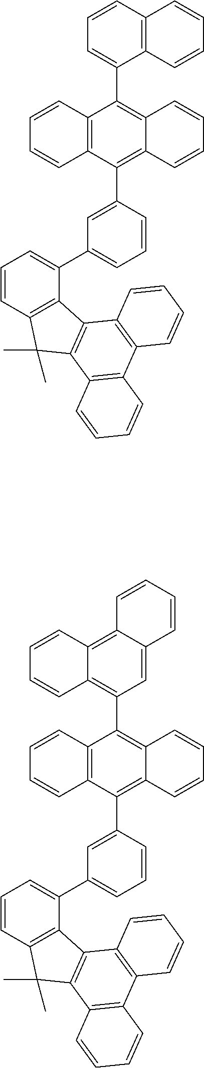

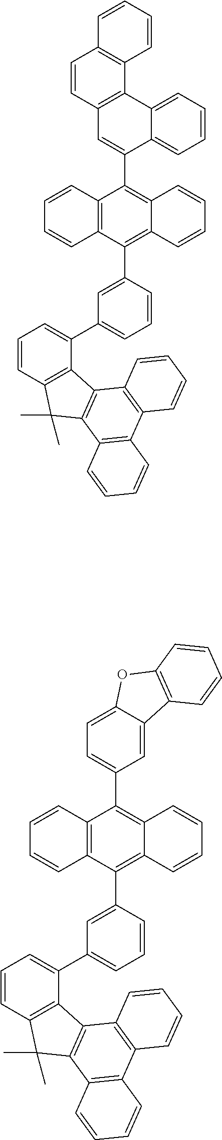

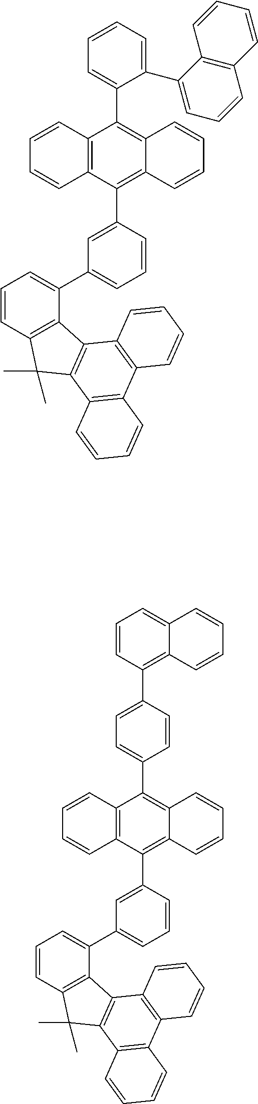

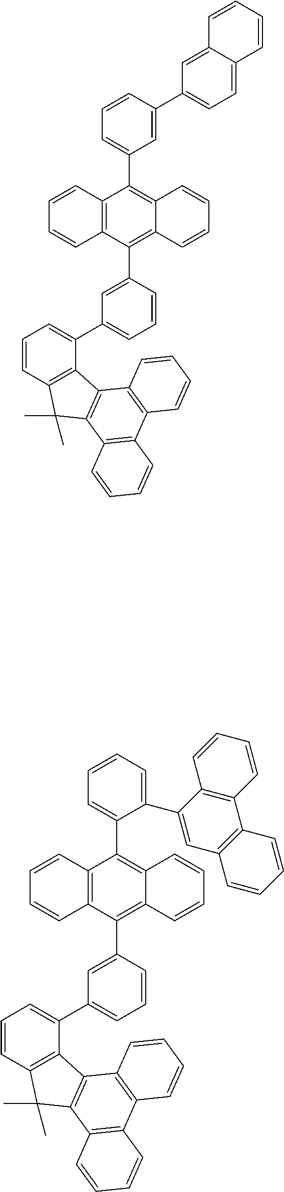

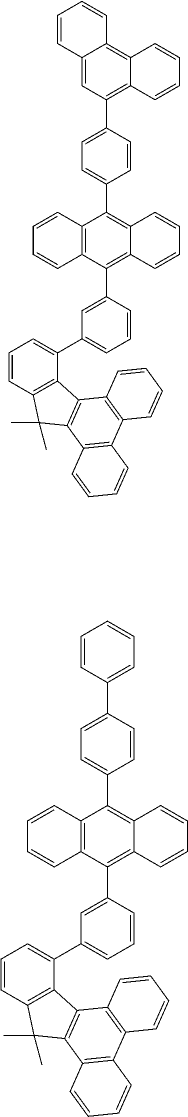

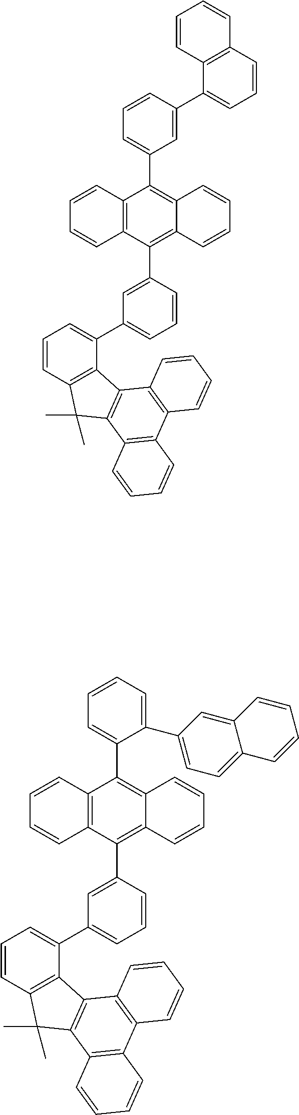

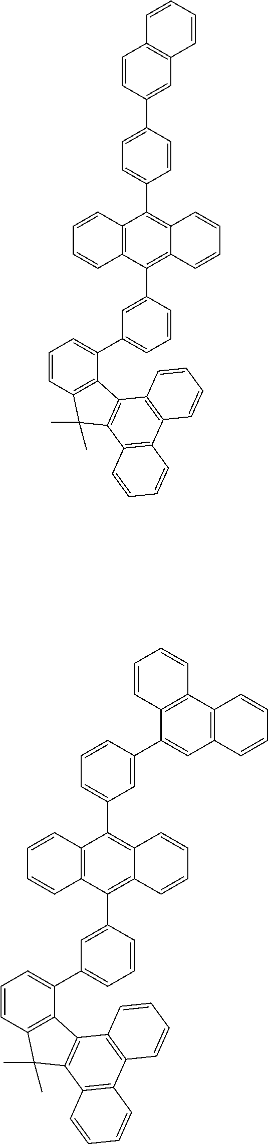

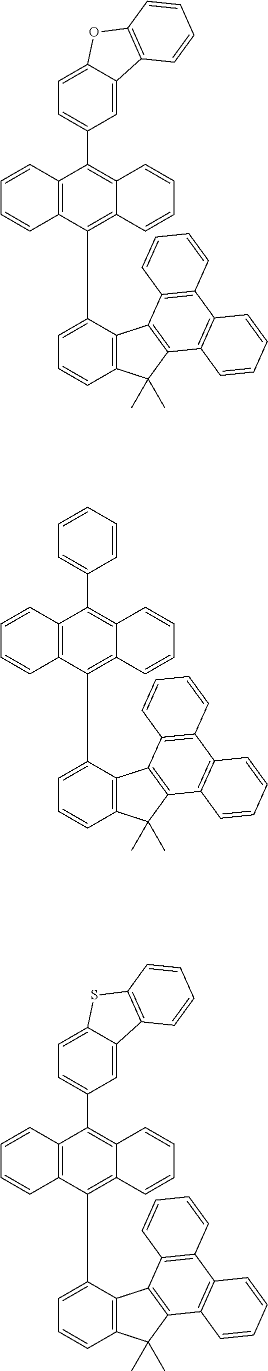

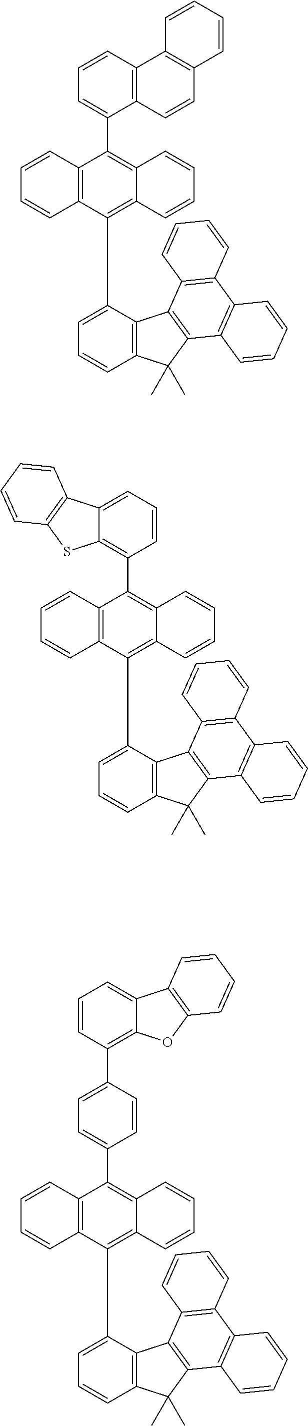

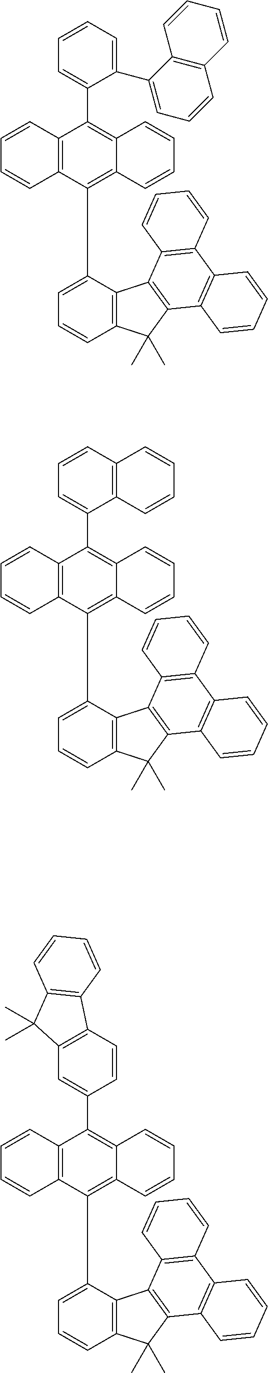

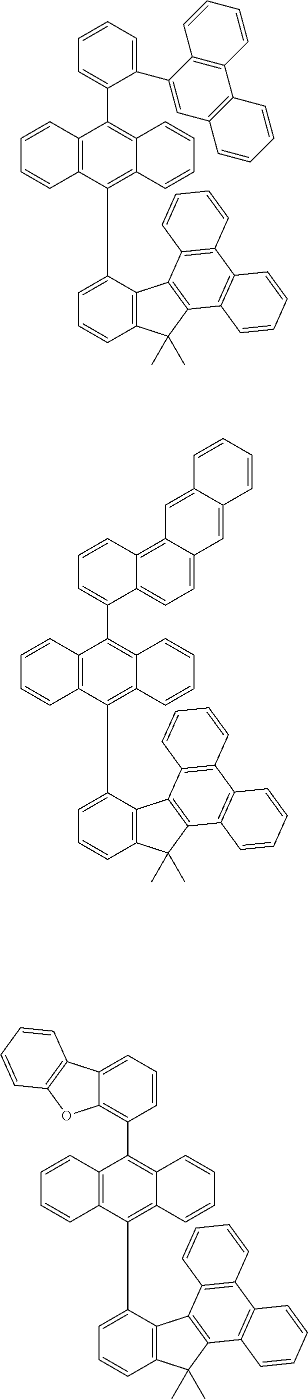

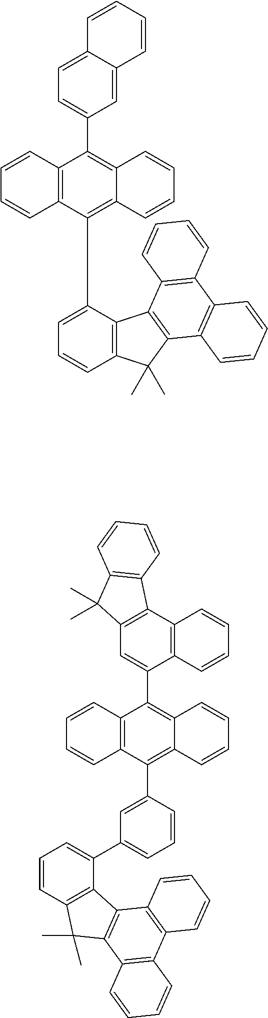

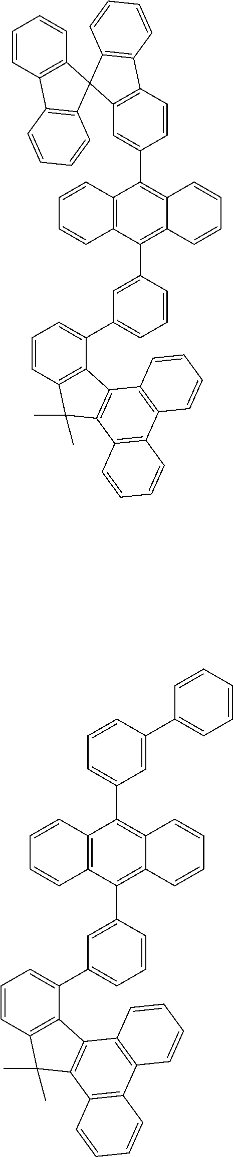

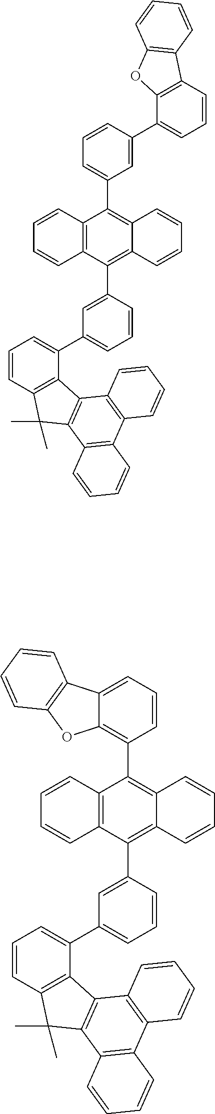 [SMILES: CC1(C)c2cccc(-c3cccc(-c4c5ccccc5c(-c5cccc(-c6cccc7c6oc6ccccc67)c5)c5ccccc45)c3)c2-c2c1c1ccccc1c1ccccc21 ; CC1(C)c2cccc(-c3cccc(-c4c5ccccc5c(-c5cccc6c5oc5ccccc56)c5ccccc45)c3)c2-c2c1c1ccccc1c1ccccc21]

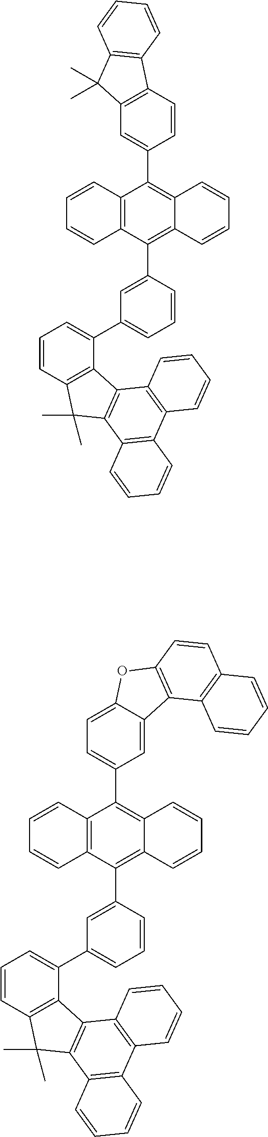

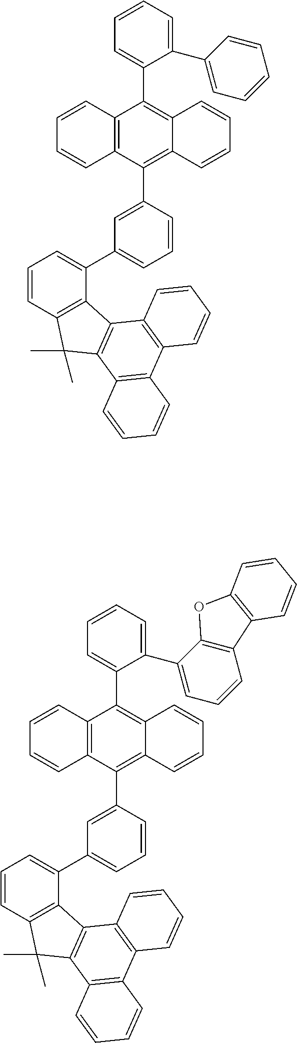

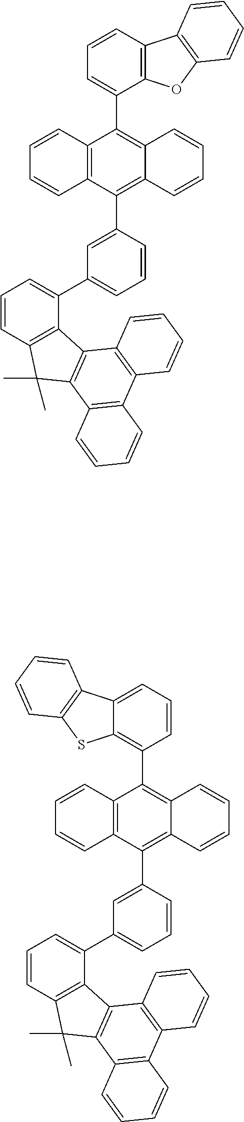 [SMILES: CC1(C)c2cccc(-c3cccc(-c4c5ccccc5c(-c5cccc6c5oc5ccccc56)c5ccccc45)c3)c2-c2c1c1ccccc1c1ccccc21 ; CC1(C)c2cccc(-c3cccc(-c4c5ccccc5c(-c5cccc6c5sc5ccccc56)c5ccccc45)c3)c2-c2c1c1ccccc1c1ccccc21]

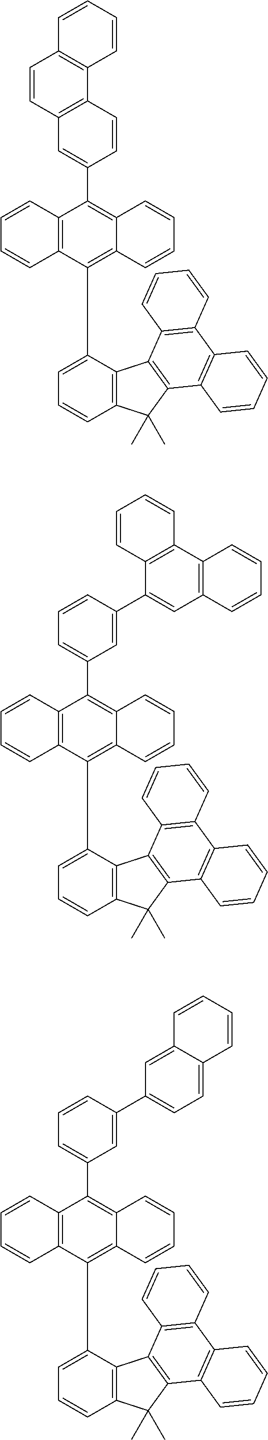

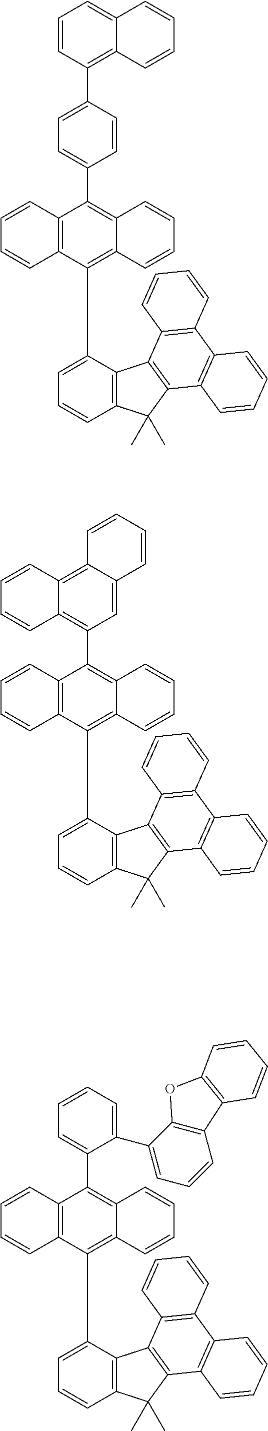

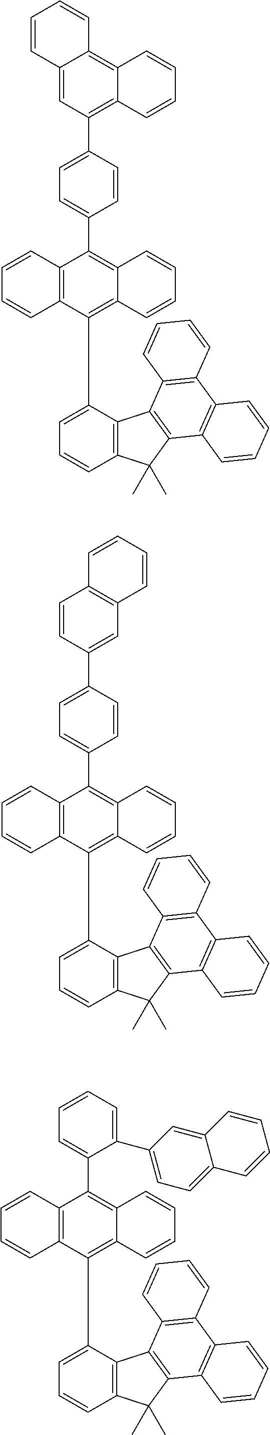

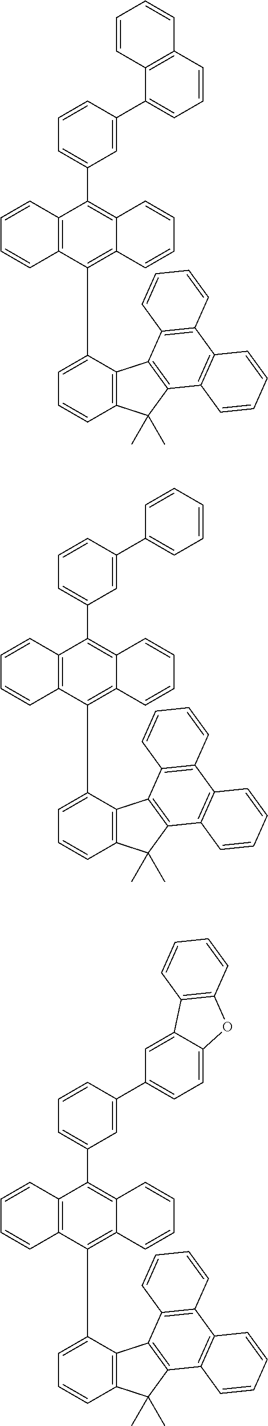

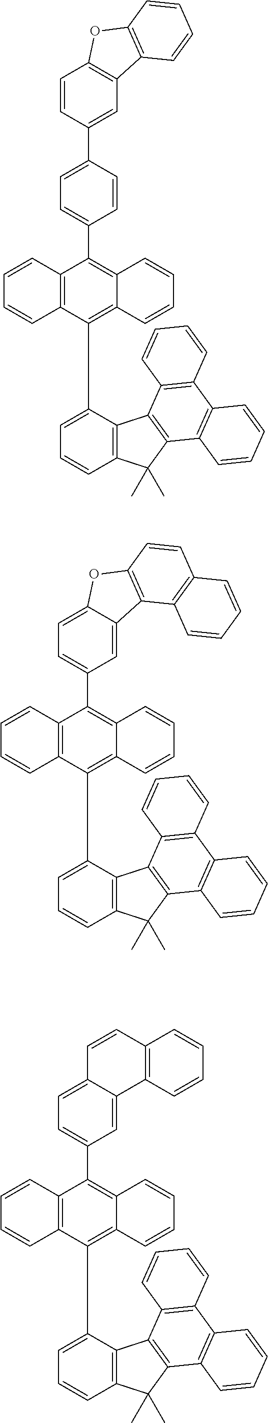

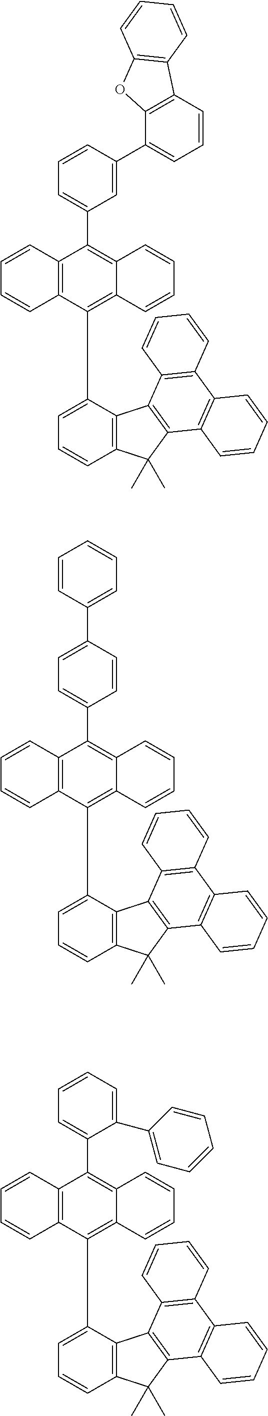

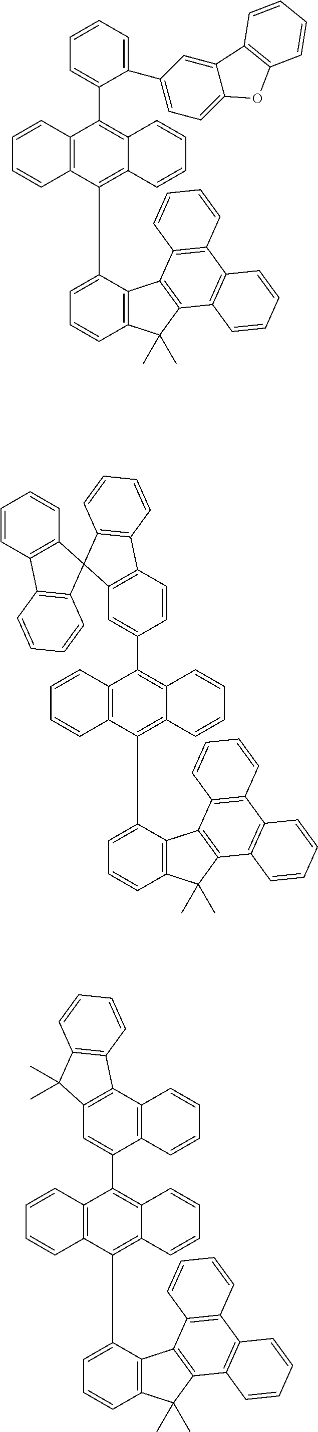

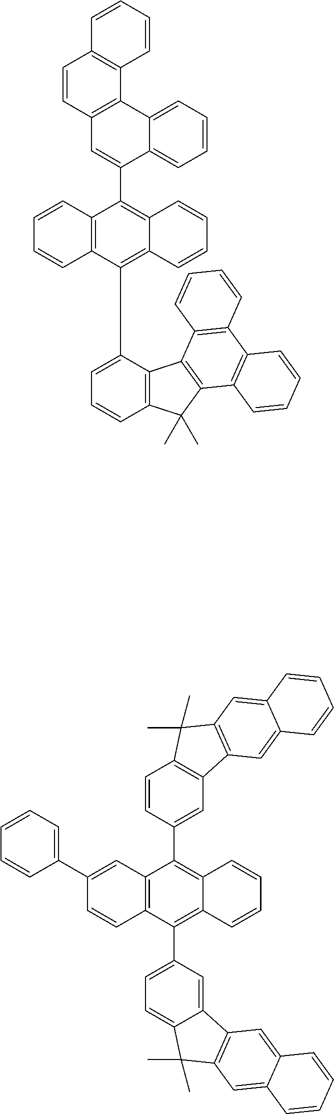

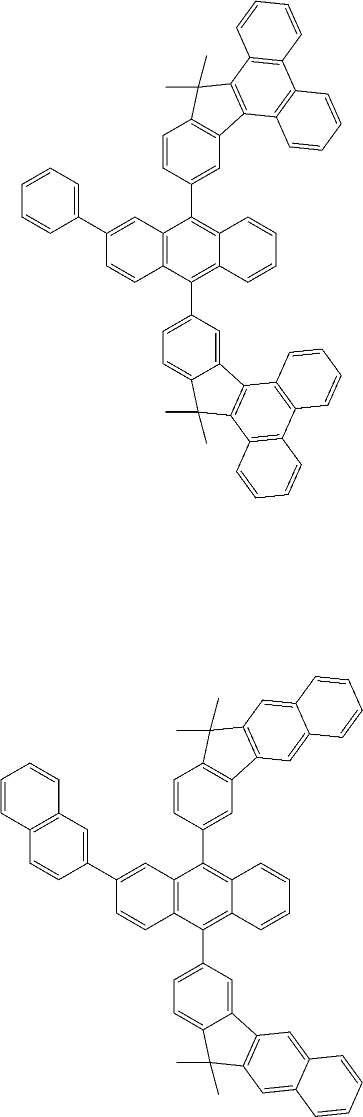

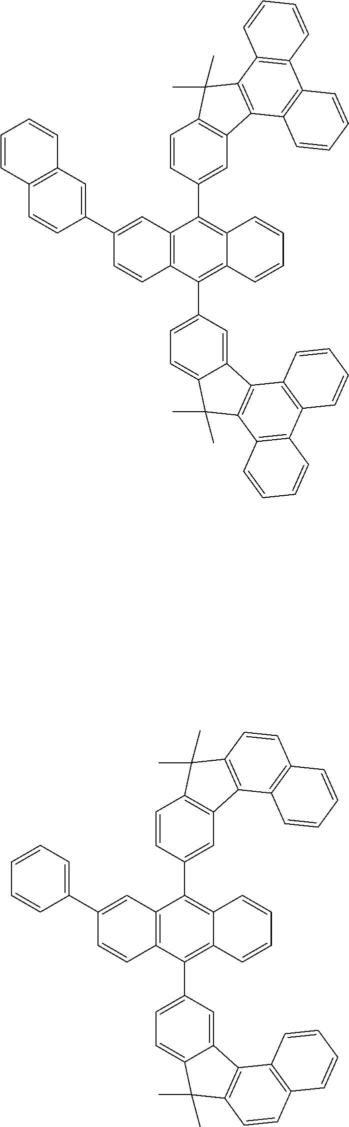

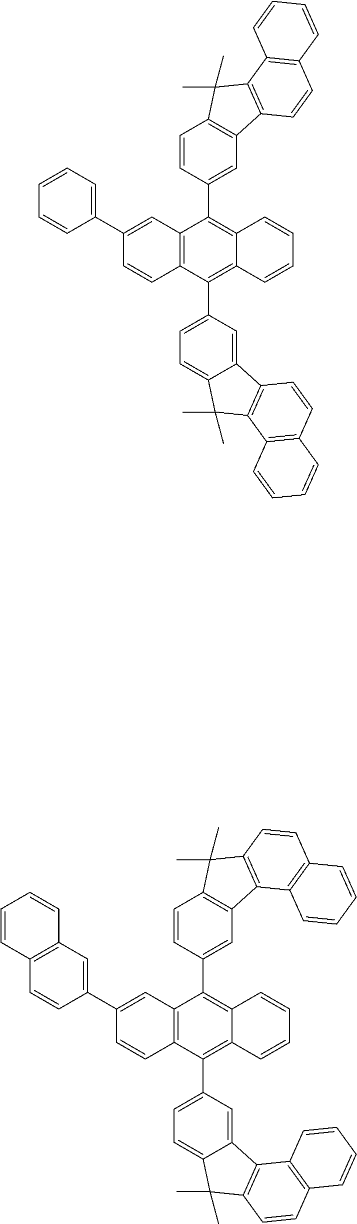

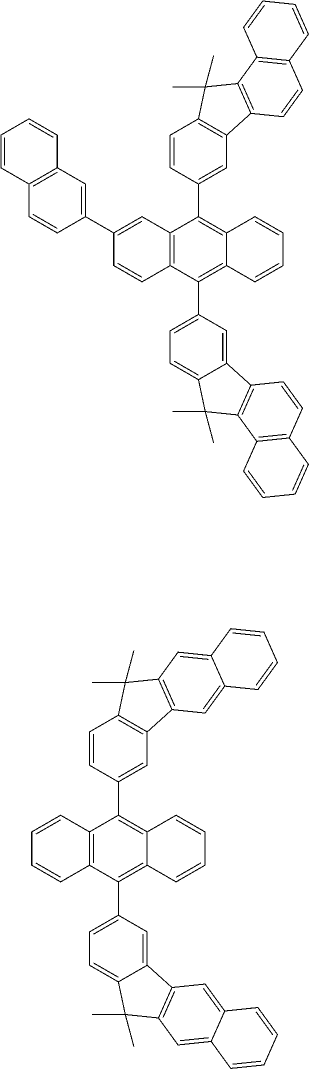

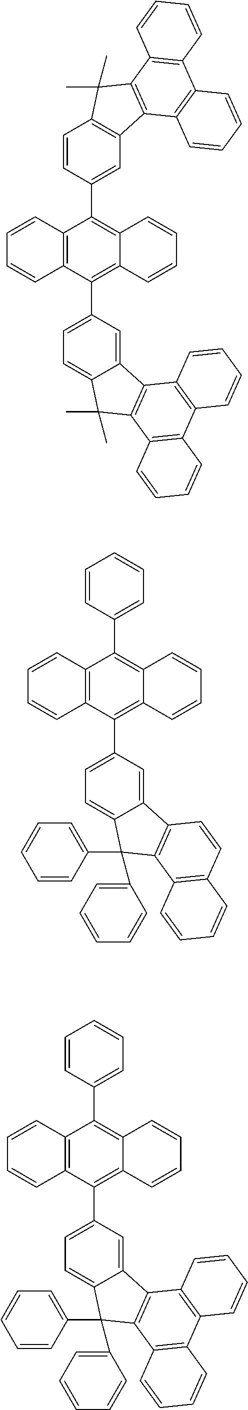

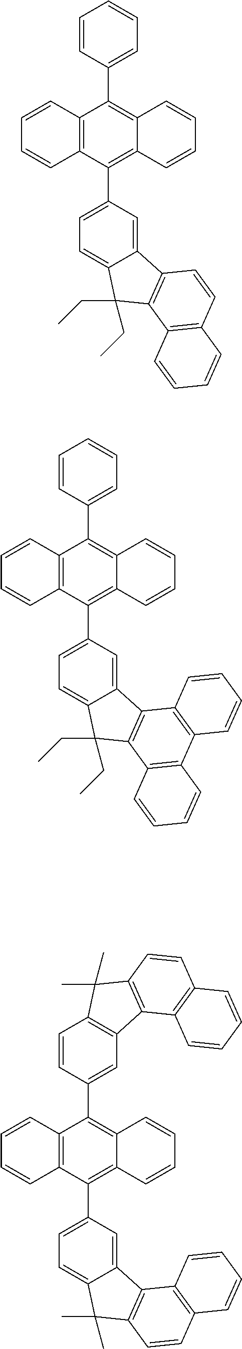

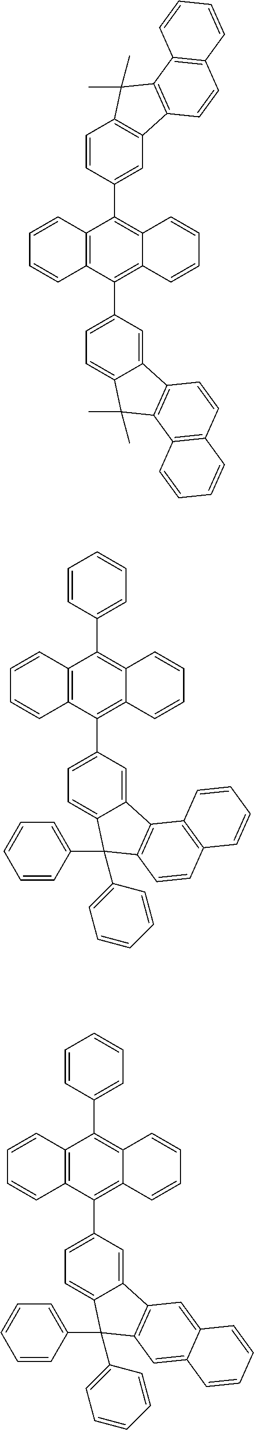

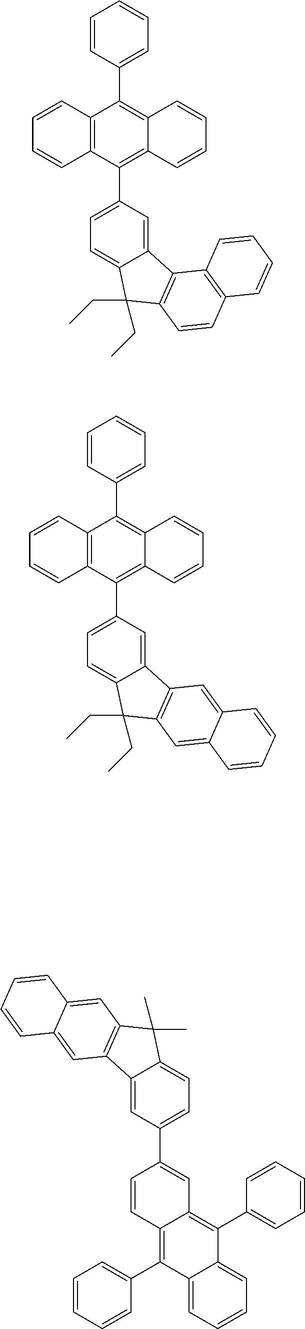

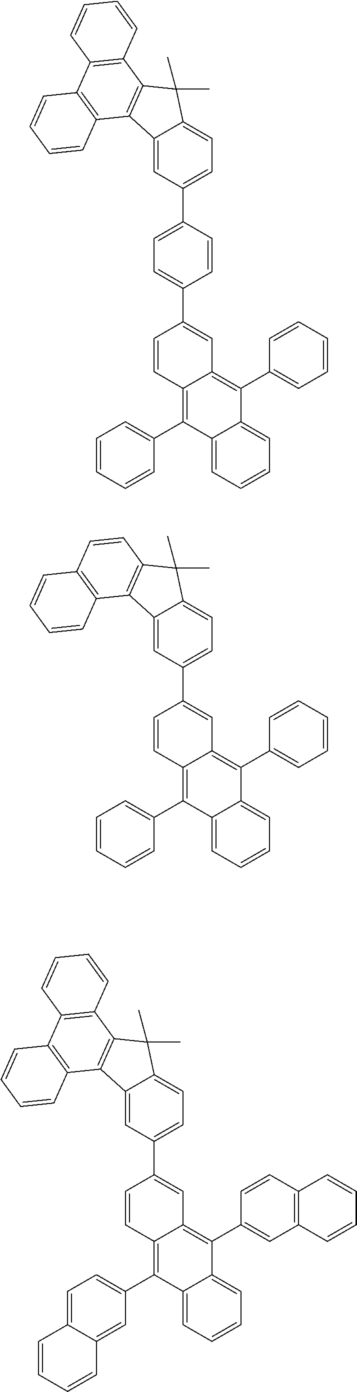

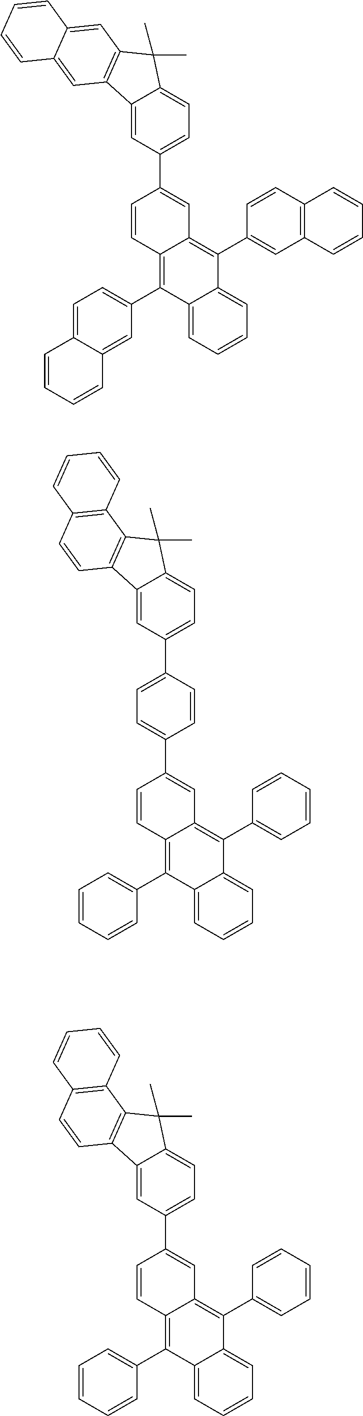

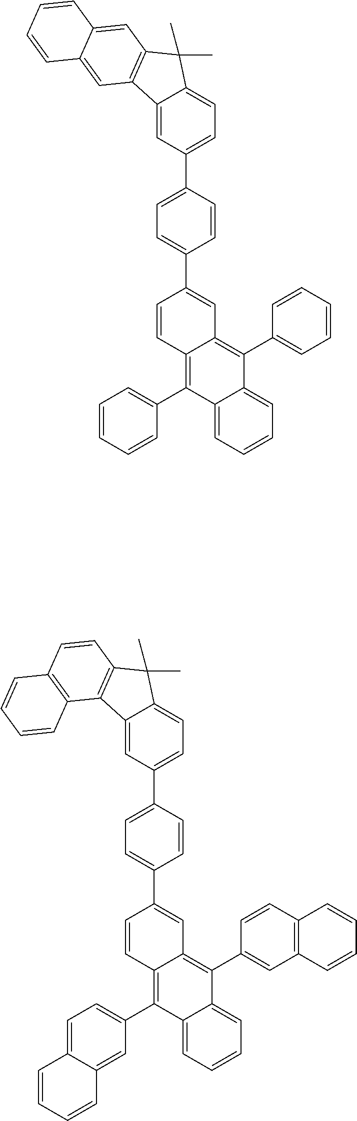

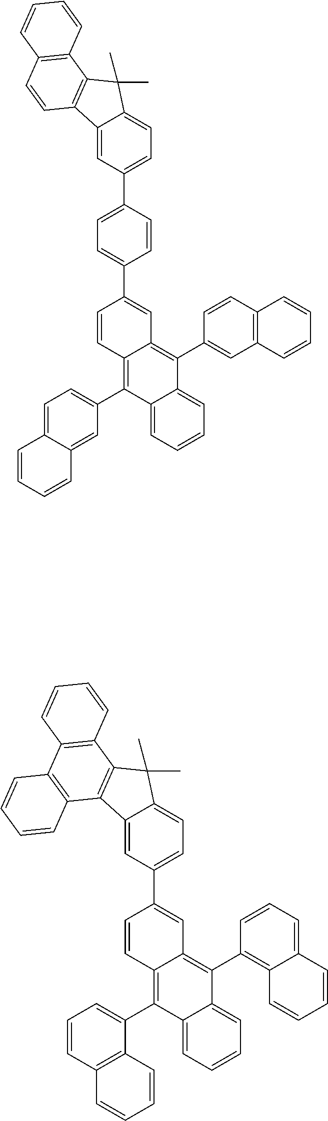

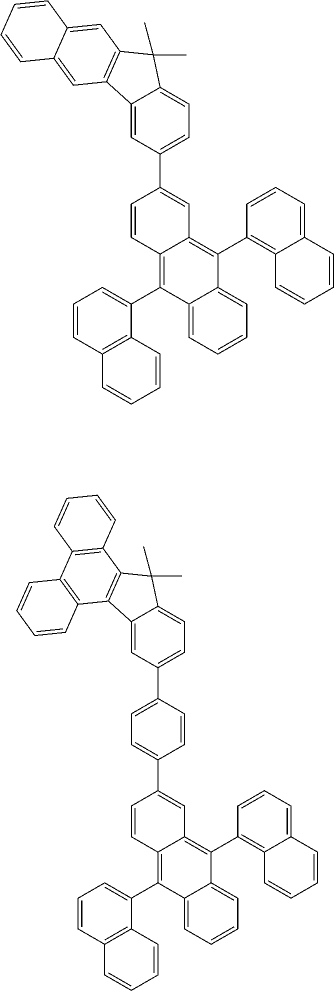

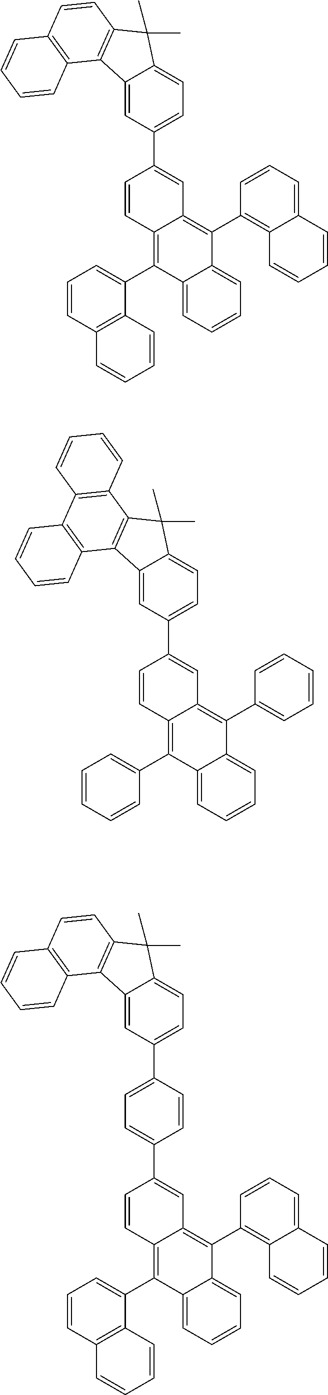

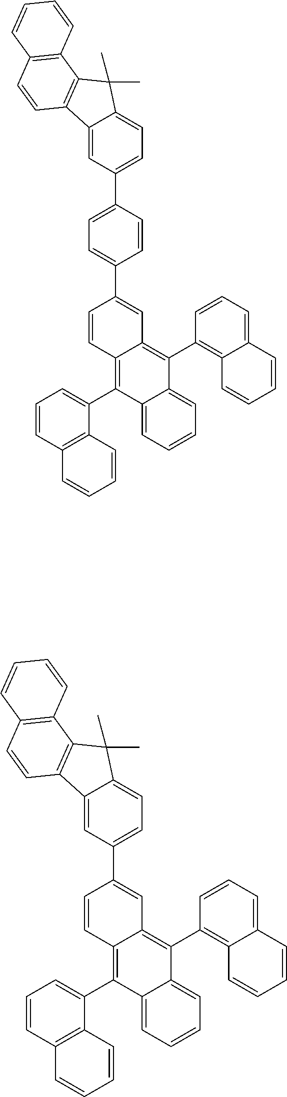

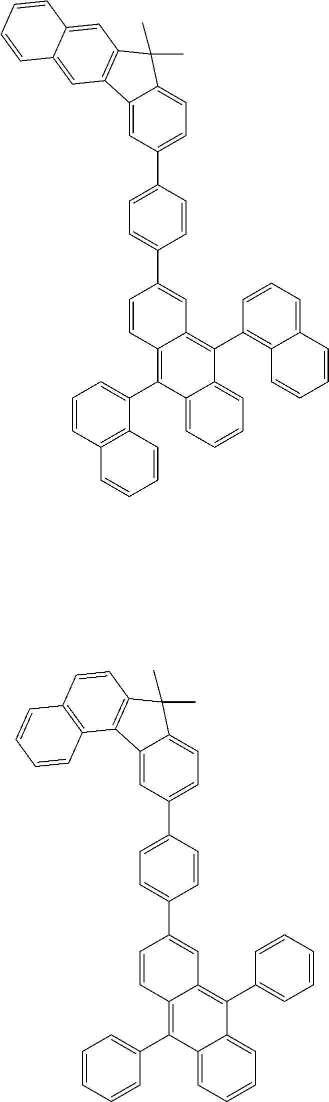

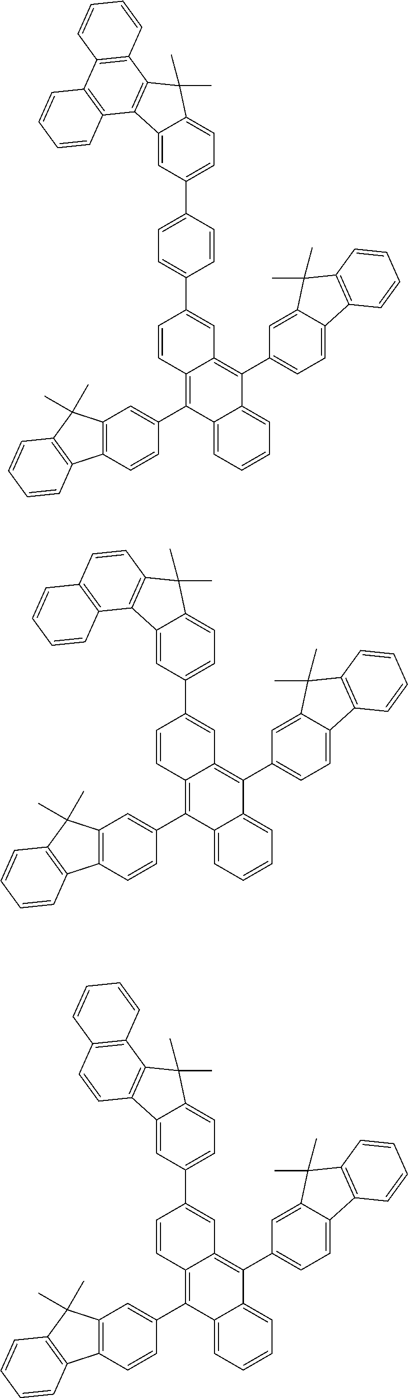

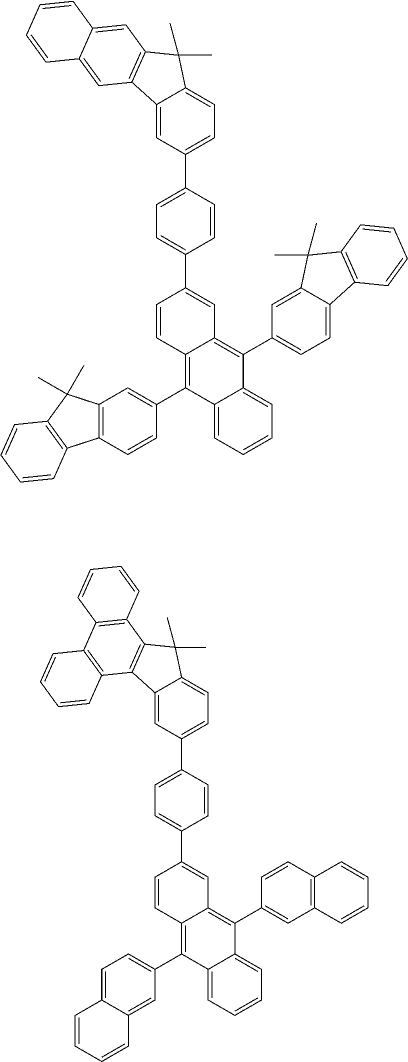

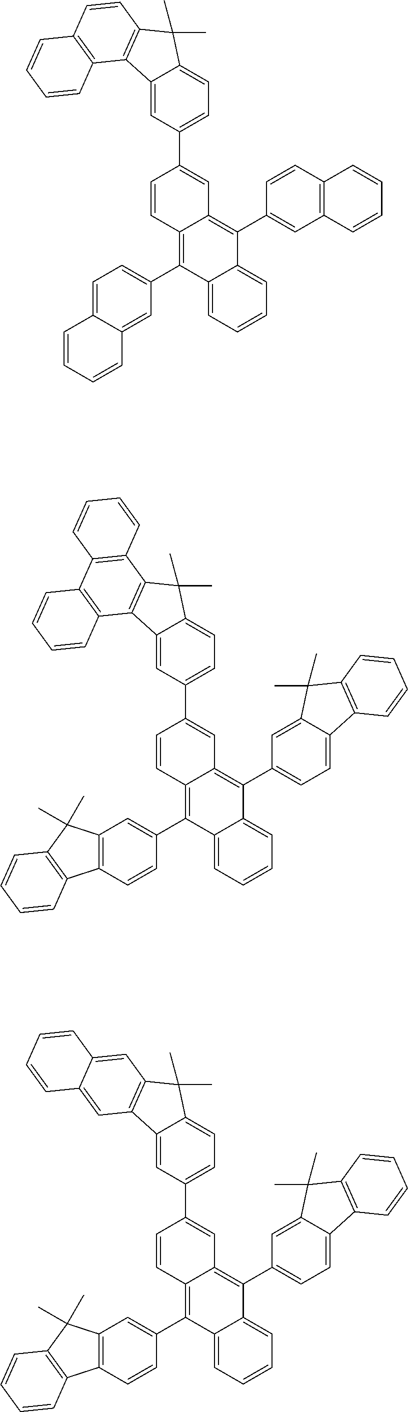

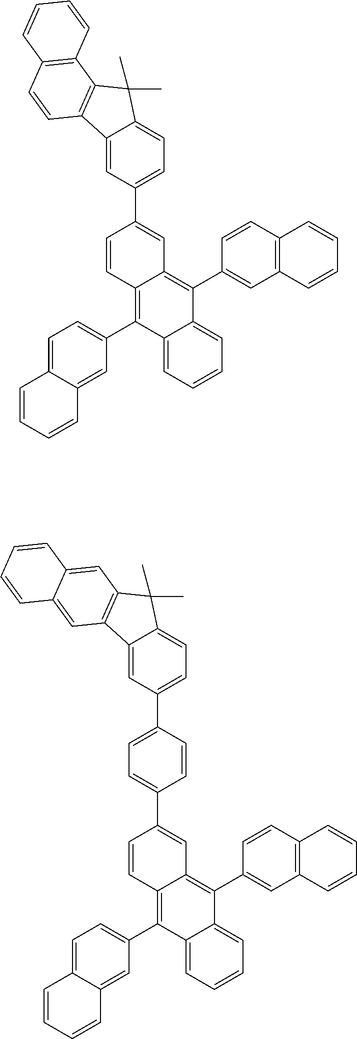

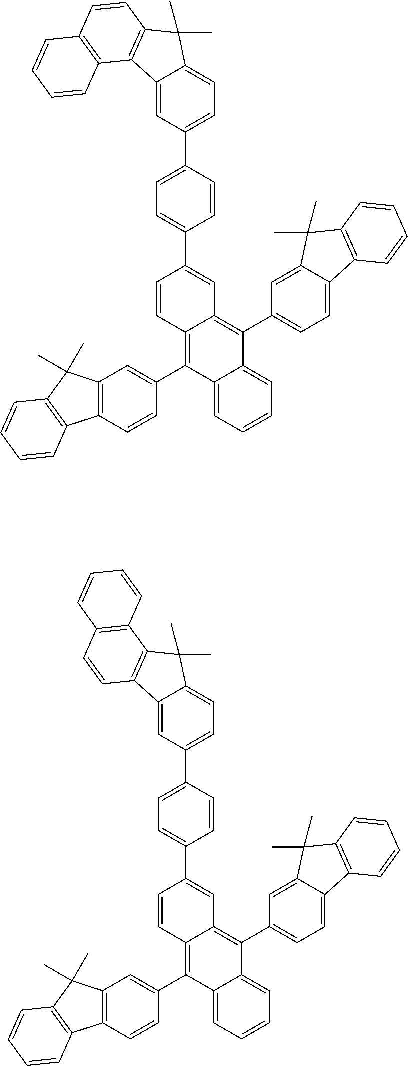

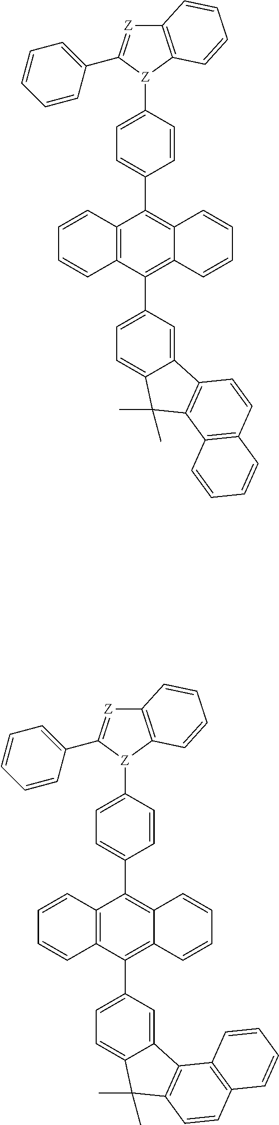

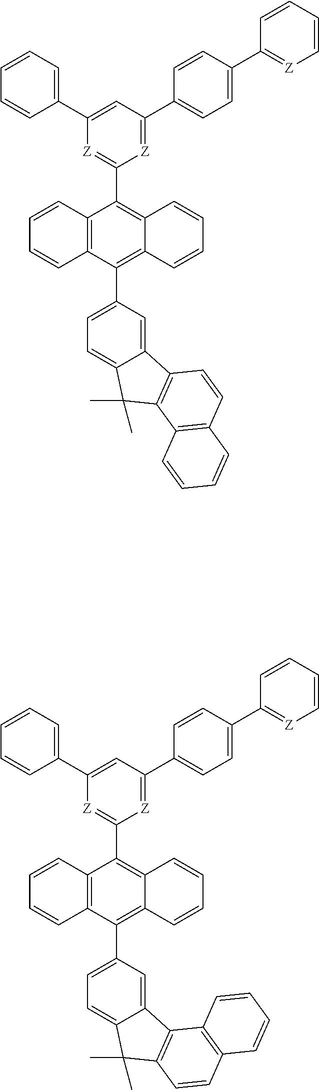

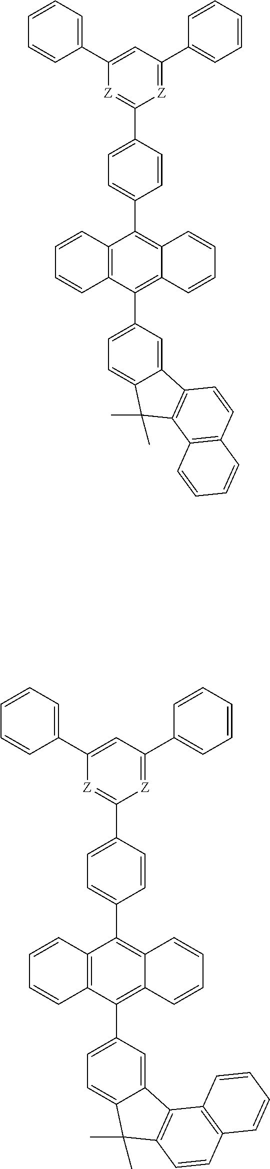

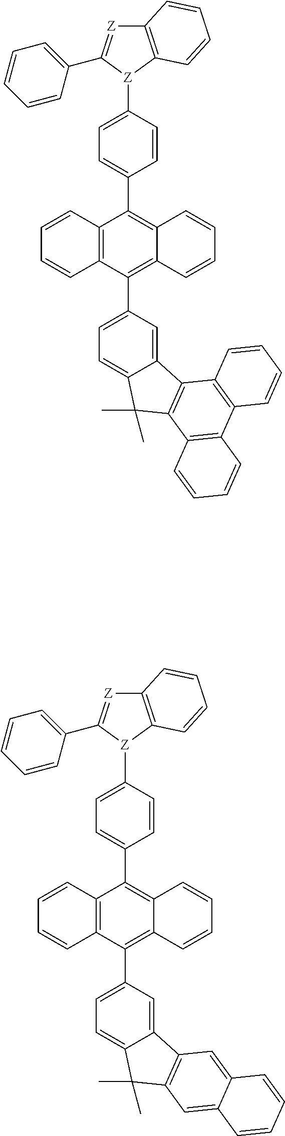

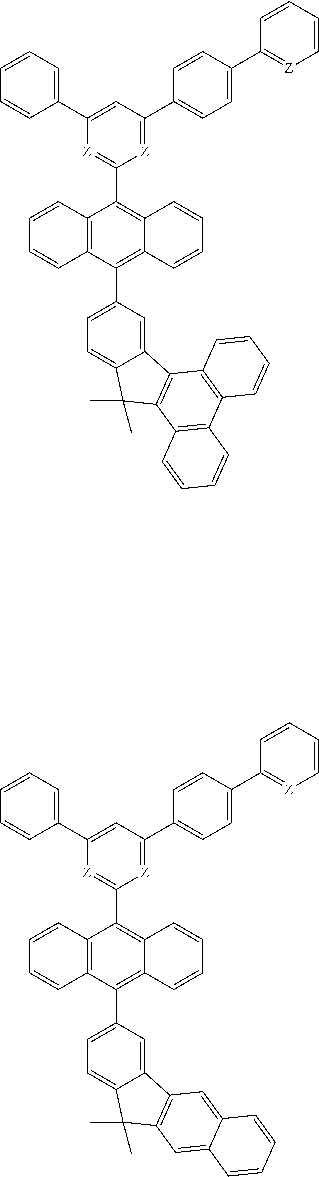

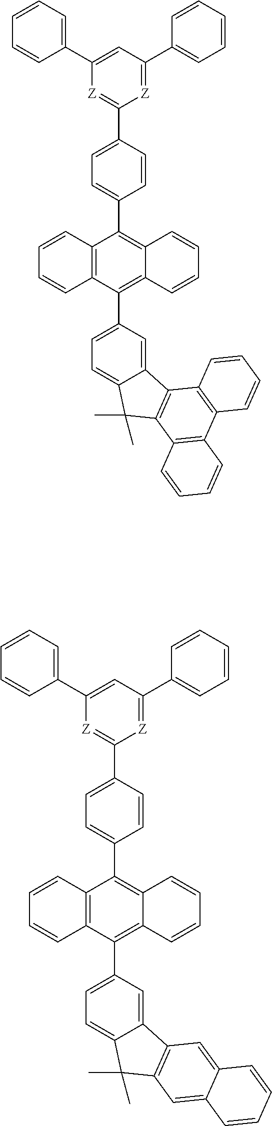

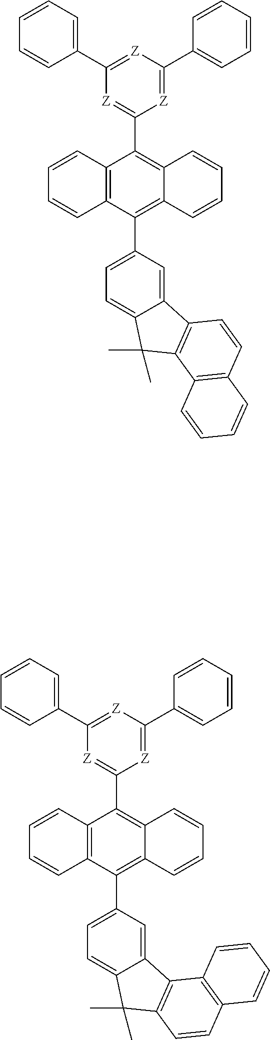

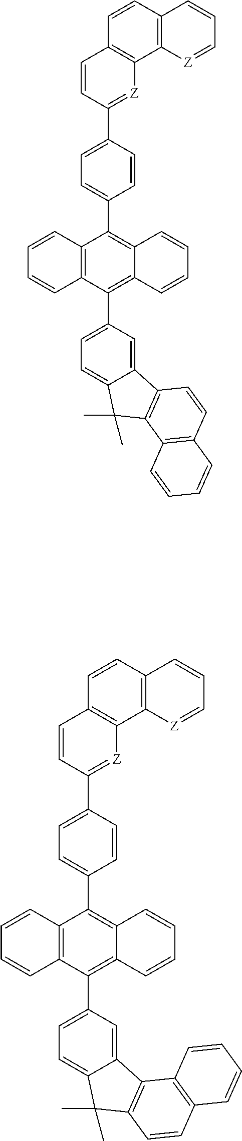

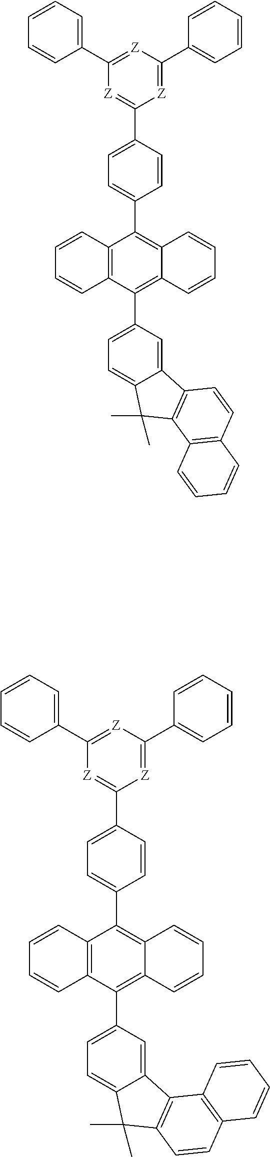

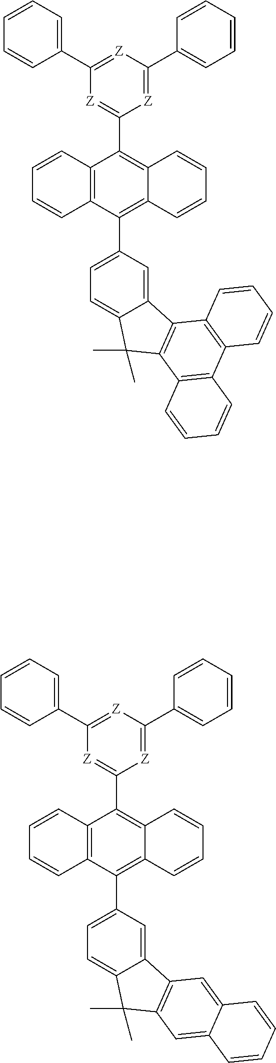

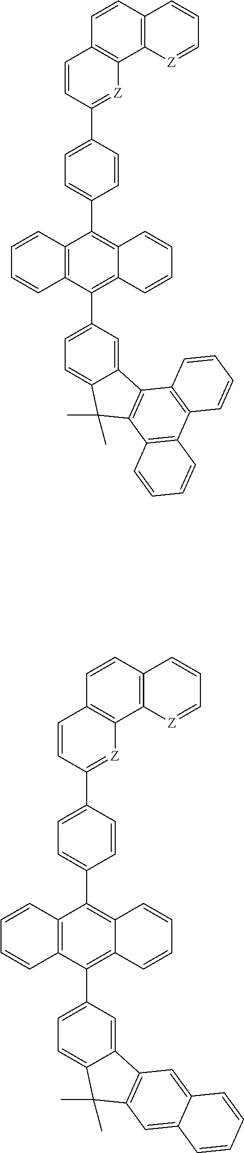

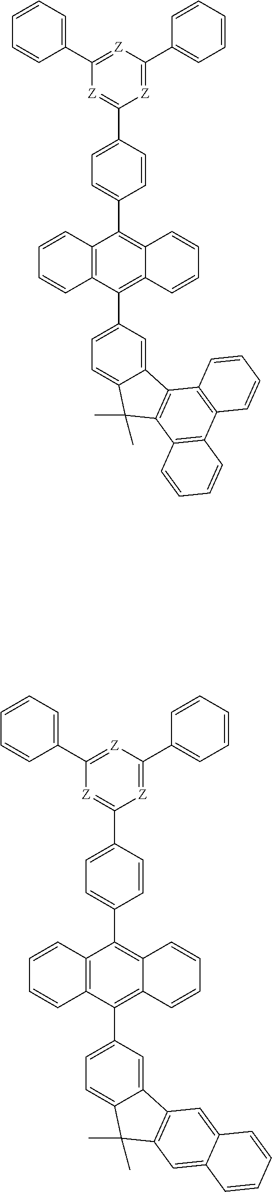

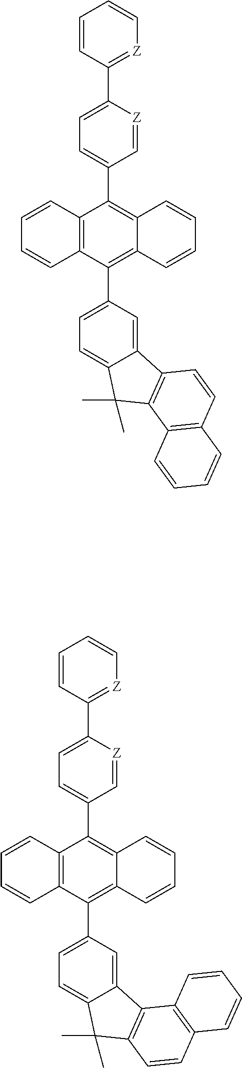

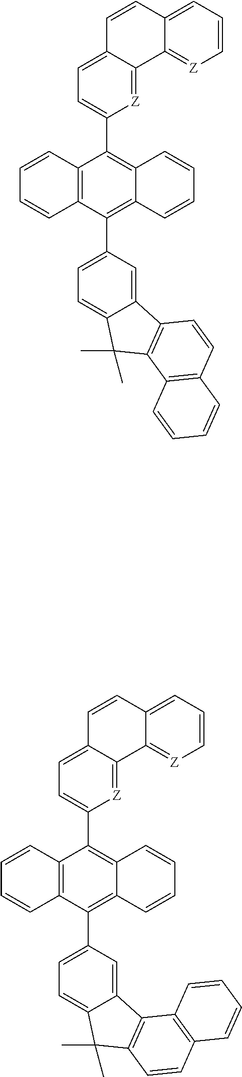

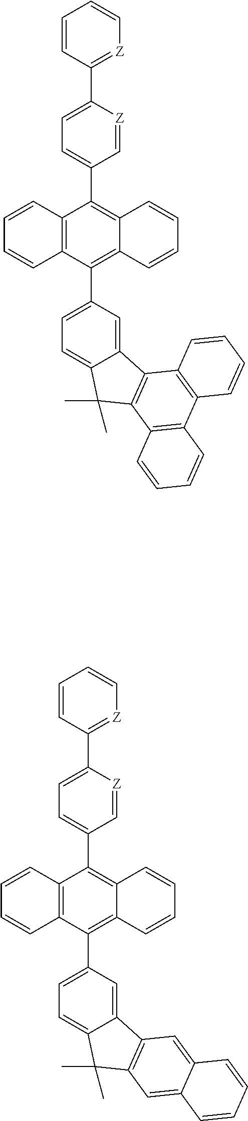

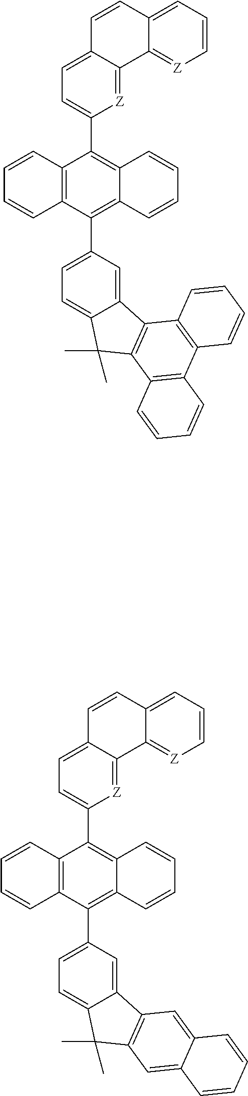

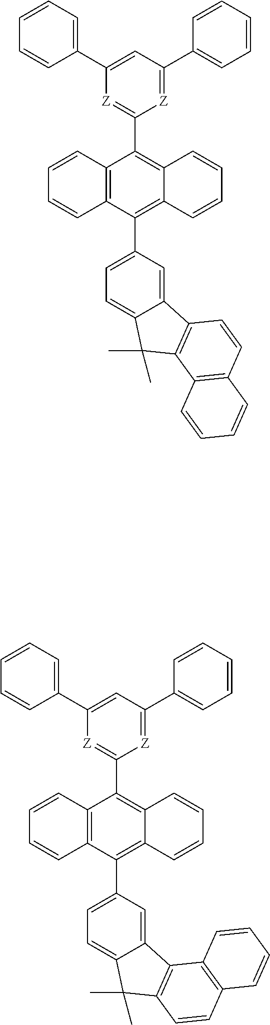

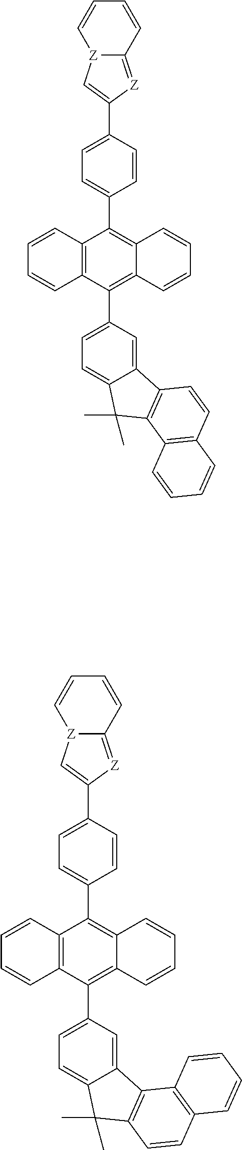

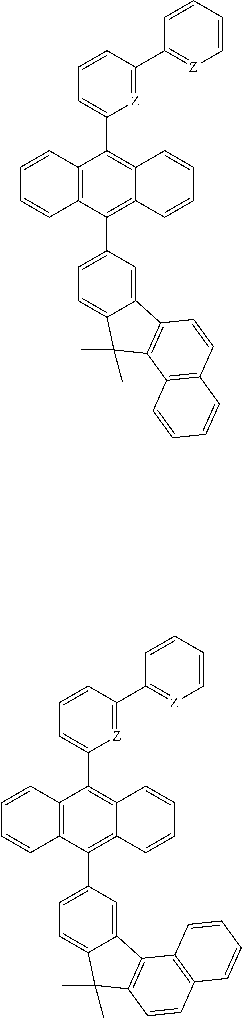

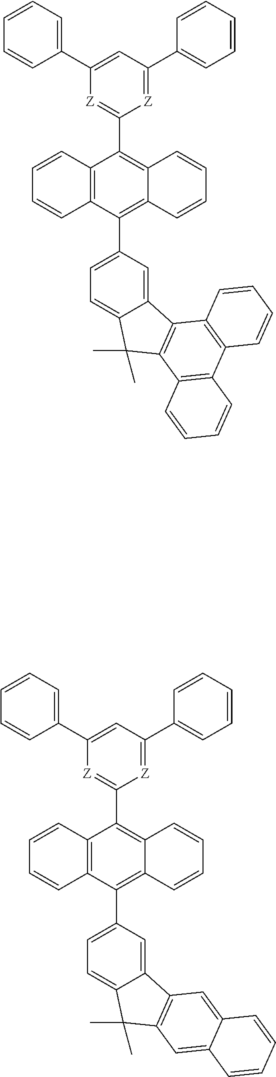

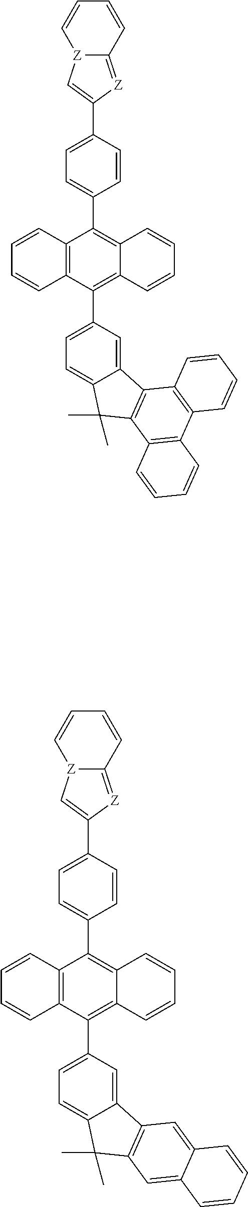

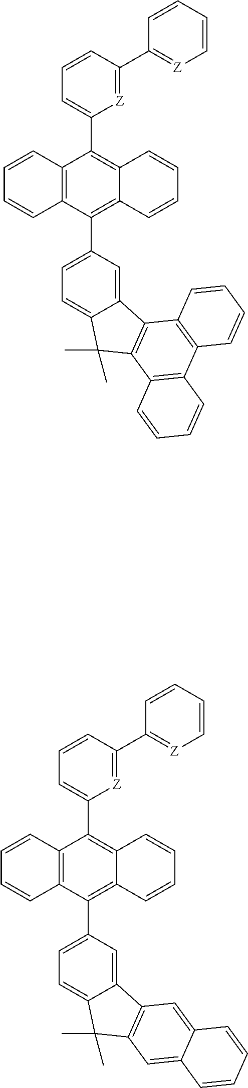

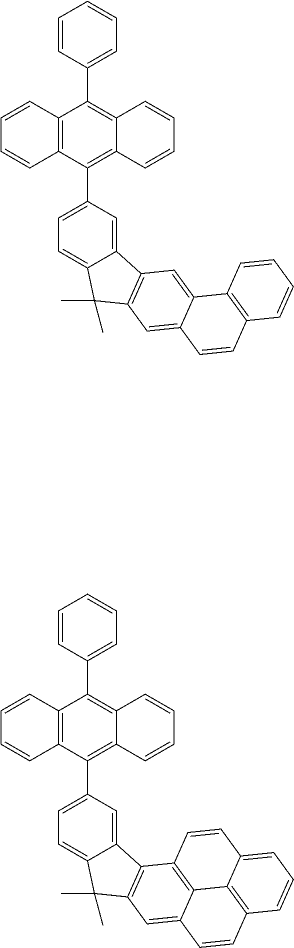

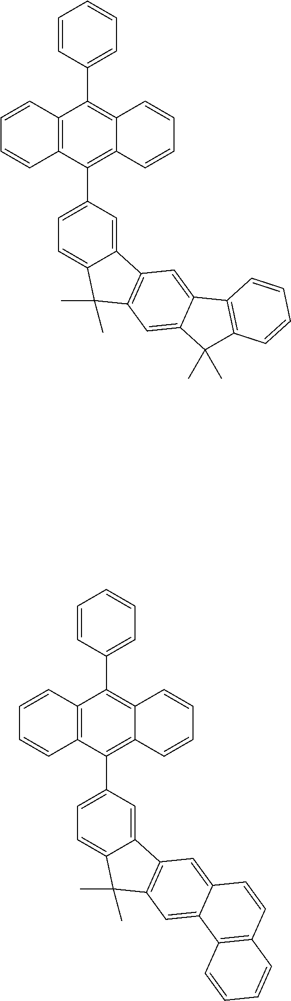

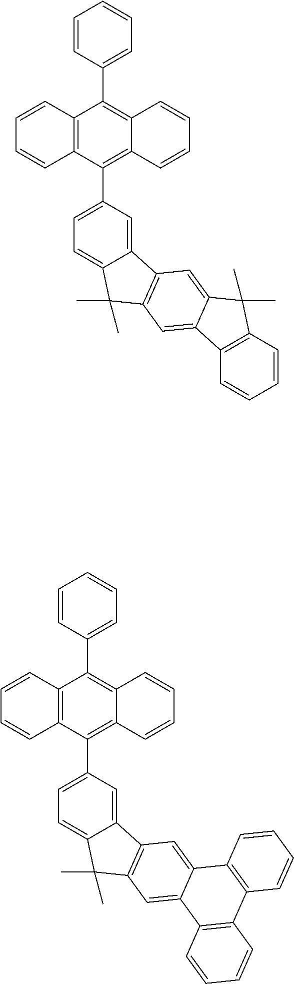

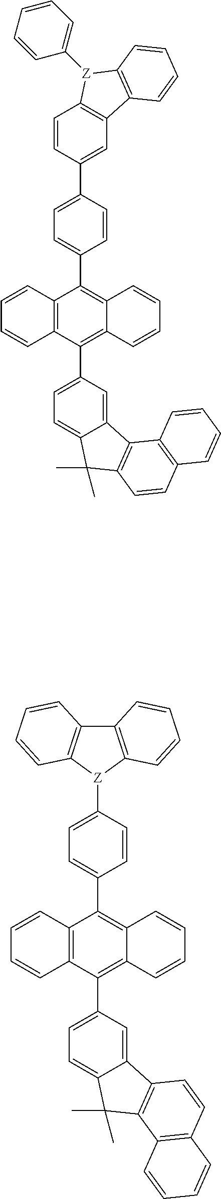

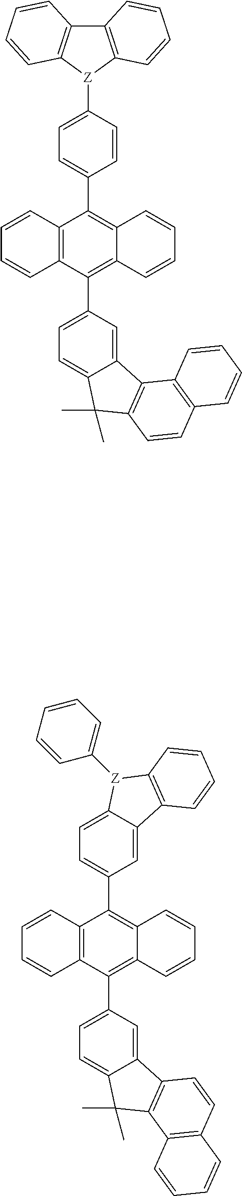

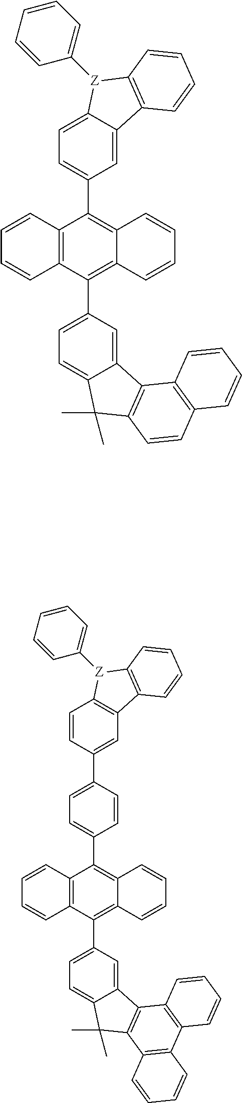

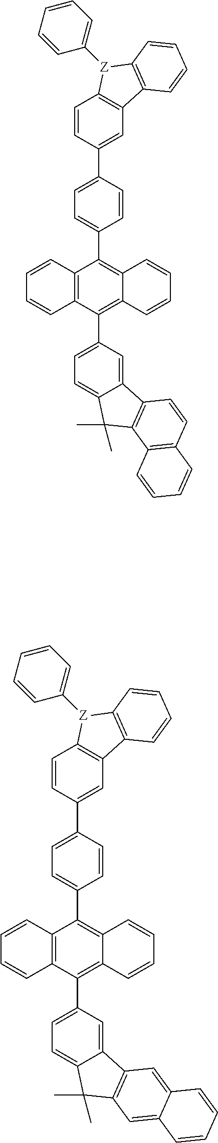

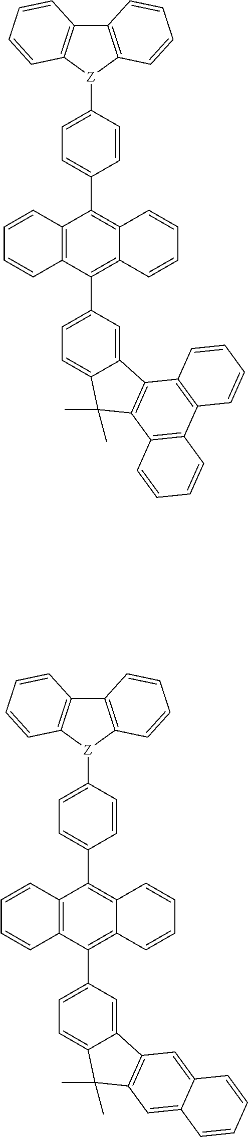

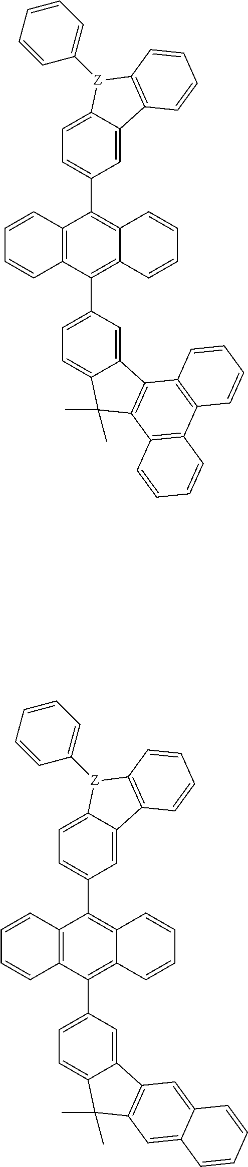

The above compound may be used as a material for producing an organic EL device and an emitting material for producing an organic EL device.

An organic electroluminescence (EL) device according to one aspect of the invention includes a cathode, an anode, and one or more organic thin film layers that are provided between the cathode and the anode, the one or more organic thin film layers including an emitting layer, and at least one organic thin film layer included in the one or more organic thin film layers including the anthracene derivative according to one aspect of the invention either alone or as a component of a mixture.

It is preferable that the emitting layer include the anthracene derivative. The anthracene derivative is preferably included in the emitting layer as a host material.

When the organic EL device includes a plurality of organic thin film layers, the organic EL device may have an (anode/hole-injecting layer/emitting layer/cathode) stacked structure, an (anode/emitting layer/electron-injecting layer/cathode) stacked structure, an (anode/hole-injecting layer/emitting layer/electron-injecting layer/cathode) stacked structure, an (anode/hole-injecting layer/hole-transporting layer/emitting layer/electron-injecting layer/cathode) stacked structure, or the like.

The anthracene derivative may be used for an arbitrary organic layer in the organic EL device. Note that it is preferable that an emitting part include the anthracene derivative. It is particularly preferable that the emitting layer include the anthracene derivative. The content of the anthracene derivative is not particularly limited, and may be appropriately adjusted. The content of the anthracene derivative is normally 1 to 100 mass %, and preferably 30 to 100 mass %.

When the organic EL device includes a plurality of organic thin film layers, a decrease in luminance or lifetime due to quenching can be prevented. An emitting material, a doping material, a hole-injecting material, and an electron-injecting material may optionally be used in combination. The luminance or the luminous efficiency may be improved depending on the doping material. The hole-injecting layer, the emitting layer, and the electron-injecting layer may respectively include two or more layers. When the hole-injecting layer includes two or more layers, a layer into which holes are injected from the electrode is referred to as “hole-injecting layer”, and a layer that receives holes from the hole-injecting layer, and transports the holes to the emitting layer is referred to as “hole-transporting layer”. Likewise, when the electron-injecting layer includes two or more layers, a layer into which electrons are injected from the electrode is referred to as “electron-injecting layer”, and a layer that receives electrons from the electron-injecting layer, and transports the electrons to the emitting layer is referred to as “electron-transporting layer”. Each layer is selected taking account of the energy level of the material, the heat resistance of the material, the adhesion of the material to an organic layer or a metal electrode, and the like.

Examples of a material that may be used for the emitting layer together with the anthracene derivative include, but are not limited to, a fused polycyclic aromatic compound (e.g., naphthalene, phenanthrene, rubrene, anthracene, tetracene, pyrene, perylene, chrysene, decacyclene, coronene, tetraphenylcyclopentadiene, pentaphenylcyclopentadiene, fluorene, and spirofluorene) and derivatives thereof, an organic metal complex (e.g., tris(8-quinolinolate)aluminum), a triarylamine derivative, a styrylamine derivative, a stilbene derivative, a coumarin derivative, a pyran derivative, an oxazone derivative, a benzothiazole derivative, a benzoxazole derivative, a benzimidazole derivative, a pyrazine derivative, a cinnamate derivative, a diketopyrrolopyrrole derivative, an acridone derivative, a quinacridone derivative, and the like.

The emitting layer included in the organic EL device may include an emitting dopant (phosphorescent dopant and/or fluorescent dopant) in addition to the emitting material. An emitting layer that includes the emitting dopant may be stacked on an emitting layer that includes above compound.

The term “fluorescent dopant” refers to a compound that emits light due to singlet excitons. The fluorescent dopant is preferably a compound that is selected from an amine-based compound, an aromatic compound, a chelate complex such as a tris(8-quinolinolato)aluminum complex, a coumarin derivative, a tetraphenylbutadiene derivative, a bisstyrylarylene derivative, an oxadiazole derivative, and the like taking account of the desired emission color. Among these, a styrylamine compound, a styryldiamine compound, an arylamine compound, an aryldiamine compound, and a fluoranthene compound are more preferable, and a fused polycyclic amine derivatives is still more preferable. These fluorescent dopants may be used either alone or in combination.

A compound represented by the following formula (A) is preferable as the fused polycyclic amine derivative.

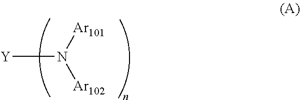

In the formula (A), Y is a substituted or unsubstituted fused aromatic hydrocarbon group including 10 to 50 ring carbon atoms.

Ar₁₀₁, and Ar₁₀₂ are independently a substituted or unsubstituted aryl group including 6 to 50 ring carbon atoms, or a substituted or unsubstituted heterocyclic group including 5 to 50 ring atoms.

Specific examples of Y include the groups mentioned above as examples of the fused aryl group. Y is preferably a substituted or unsubstituted anthryl group, a substituted or unsubstituted pyrenyl group, or a substituted or unsubstituted chrysenyl group. Specific examples of Ar₁₀₁, and Ar₁₀₂ include the same groups mentioned above as examples of the aryl group including 6 to 50 ring carbon atoms and the heterocyclic group including 5 to 50 ring atoms in the compound represented by the formula (1).

n is an integer from 1 to 4, and preferably 1 or 2.

A compound represented by the following formula (16) or (17) is preferable as the compound represented by the formula (A).

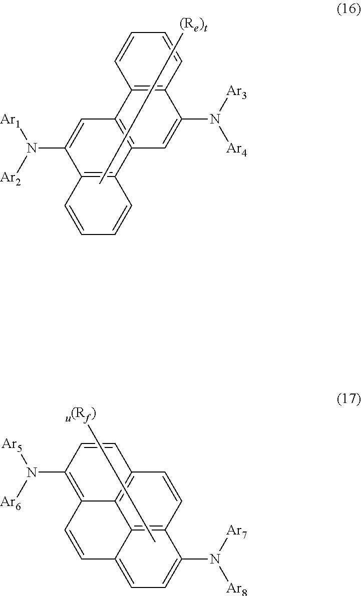

In the formulas (16) and (17), Rₑ and R_{f} are independently a substituted or unsubstituted alkyl group including 1 to 20 carbon atoms, a substituted or unsubstituted alkenyl group including 2 to 50 carbon atoms, a substituted or unsubstituted alkynyl group including 2 to 50 carbon atoms, a substituted or unsubstituted aralkyl group including 7 to 50 carbon atoms, a substituted or unsubstituted cycloalkyl group including 3 to 20 ring carbon atoms, a substituted or unsubstituted alkoxy group including 1 to 20 carbon atoms, a substituted or unsubstituted aryloxy group including 6 to 20 ring carbon atoms, a substituted or unsubstituted aryl group including 6 to 50 ring carbon atoms, a substituted or unsubstituted heterocyclic group including 5 to 50 ring atoms, a substituted or unsubstituted alkylsilyl group including 1 to 30 carbon atoms, a substituted or unsubstituted arylsilyl group including 6 to 50 ring carbon atoms, a substituted or unsubstituted alkylgermanium group including 1 to 50 carbon atoms, or a substituted or unsubstituted arylgermanium group including 6 to 50 ring carbon atoms. Rₑ and R_{f} are independently bonded to an arbitrary position of an arbitrary benzene ring that forms the fused polycyclic skeleton.

Rₑ and R_{f} are preferably a substituted or unsubstituted aryl group including 6 to 50 ring carbon atoms, and more preferably a substituted or unsubstituted phenyl group, a substituted or unsubstituted naphthyl group, or the like.

t is an integer from 0 to 10. u is an integer from 0 to 8.

A plurality of Rₑ are either identical or different when t is an integer from 2 to 10.

A plurality of R_{f} are either identical or different when u is an integer from 2 to 8.

Ar₁ to Ar₈ are independently a substituted or unsubstituted aryl group including 6 to 50 ring carbon atoms, or a substituted or unsubstituted heterocyclic group including 5 to 50 ring atoms.

Ar₁ to Ar₈ are preferably a substituted or unsubstituted phenyl group, a substituted or unsubstituted dibenzofuranyl group, or the like. Examples of a preferable substituent that may substitute on Ar₁ to Ar₈ include an alkyl group, a cyano group, and a substituted or unsubstituted silyl group.

A fused-ring amine derivative represented by the following formula (18) is also preferable used as the fluorescent dopant.

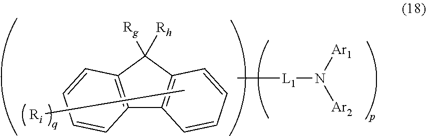

In the formula (18), R_{g} and Rₕ are independently a hydrogen atom, a substituted or unsubstituted alkyl group including 1 to 20 carbon atoms, a substituted or unsubstituted alkenyl group including 2 to 50 carbon atoms, a substituted or unsubstituted alkynyl group including 2 to 50 carbon atoms, a substituted or unsubstituted aralkyl group including 7 to 50 carbon atoms, a substituted or unsubstituted cycloalkyl group including 3 to 20 ring carbon atoms, a substituted or unsubstituted alkoxy group including 1 to 20 carbon atoms, a substituted or unsubstituted aryloxy group including 6 to 20 ring carbon atoms, a substituted or unsubstituted aryl group including 6 to 50 ring carbon atoms, a substituted or unsubstituted heterocyclic group including 5 to 50 ring atoms, a substituted or unsubstituted alkylsilyl group including 1 to 30 carbon atoms, a substituted or unsubstituted arylsilyl group including 6 to 50 ring carbon atoms, a substituted or unsubstituted alkylgermanium group including 1 to 50 carbon atoms, or a substituted or unsubstituted arylgermanium group including 6 to 50 ring carbon atoms.

Rᵢ is a substituted or unsubstituted alkyl group including 1 to 20 carbon atoms, a substituted or unsubstituted alkenyl group including 2 to 50 carbon atoms, a substituted or unsubstituted alkynyl group including 2 to 50 carbon atoms, a substituted or unsubstituted aralkyl group including 7 to 50 carbon atoms, a substituted or unsubstituted cycloalkyl group including 3 to 20 ring carbon atoms, a substituted or unsubstituted alkoxy group including 1 to 20 carbon atoms, a substituted or unsubstituted aryloxy group including 6 to 20 ring carbon atoms, a substituted or unsubstituted aryl group including 6 to 50 ring carbon atoms, a substituted or unsubstituted heterocyclic group including 5 to 50 ring atoms, a substituted or unsubstituted alkylsilyl group including 1 to 30 carbon atoms, a substituted or unsubstituted arylsilyl group including 6 to 50 ring carbon atoms, a substituted or unsubstituted alkylgermanium group including 1 to 50 carbon atoms, or a substituted or unsubstituted arylgermanium group including 6 to 50 ring carbon atoms. Rᵢ is bonded to an arbitrary position of the fluorene skeleton in the formula (18).

q is an integer from 0 to 7. A plurality of Rᵢ are either identical or different when q is an integer from 2 to 7, and adjacent Rare optionally bonded to each other to form a ring.

L₁ is a single bond or a linking group. L₁ is bonded to the fluorene skeleton in the formula (18) at a position at which Rᵢ is not bonded.

Ar₁ and Ar₂ are independently a substituted or unsubstituted aryl group including 6 to 50 ring carbon atoms, or a substituted or unsubstituted heterocyclic group including 5 to 50 ring atoms.

p is an integer from 1 to 4.

Examples of the alkyl group, the alkenyl group, the alkynyl group, the alkoxy group, the aryloxy group, the aryl group, the alkylsilyl group, and the arylsilyl group that may be included in the compounds respectively represented by the formulas (16) to (18) include those mentioned above.

The aralkyl group is represented by —Y—Z. Examples of Y include alkylene groups that correspond to the groups mentioned above as examples of the alkyl group. Examples of Z include the groups mentioned above as examples of the aryl group. The number of carbon atoms included in the aralkyl group is preferably 7 to 50 (i.e., the number of carbon atoms included in the aryl moiety is 6 to 49 (preferably 6 to 30, more preferably 6 to 20, and particularly preferably 6 to 12), and the number of carbon atoms included in the alkyl moiety is 1 to 44 (preferably 1 to 30, more preferably 1 to 20, still more preferably 1 to 10, and particularly preferably 1 to 6)). Examples of the aralkyl group include a benzyl group, a phenylethyl group, and a 2-phenylpropan-2-yl group.

Examples of the cycloalkyl group include a cycloalkyl group including 3 to 20 (preferably 3 to 10, and more preferably 3 to 8) ring carbon atoms, such as a cyclopropyl group, a cyclobutyl group, a cyclopentyl group, a cyclohexyl group, a 4-methylcyclohexyl group, an adamantyl group, and a norbornyl group.

Examples of the alkylgermanium group include a methylhydrogermyl group, a trimethylgermyl group, a triethylgermyl group, a tripropylgermyl group, a dimethyl-t-butylgermyl group, and the like.

Examples of the arylgermanium group include a phenyldihydrogermyl group, a diphenylhydrogermyl group, a triphenylgermyl group, a tritolylgermyl group, a trinaphthylgermyl group, and the like.

A compound represented by the following formula (A) and a compound represented by the following formula (B) are preferable as the styrylamine compound and the styryldiamine compound.

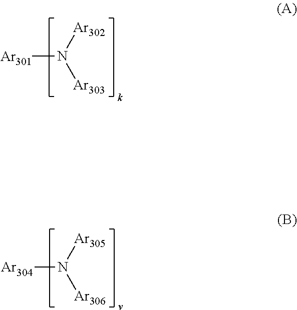

In the formula (A), Ar₃₀₁ is a k-valent group that corresponds to a phenyl group, a naphthyl group, a biphenyl group, a terphenyl group, a stilbene group, a styrylaryl group, or a distyrylaryl group, and Ar₃₀₂ and Ar₃₀₃ are independently an aryl group including 6 to 20 ring carbon atoms. Ar₃₀₁, Ar₃₀₂, and Ar₃₀₃ are either substituted or unsubstituted.

k is an integer from 1 to 4, and preferably 1 or 2. One of Ar₃₀₁ to Ar₃₀₃ is a group that includes a styryl group. It is more preferable that at least one of Ar₃₀₂ and Ar₃₀₃ be substituted with a styryl group.

Examples of the aryl group including 6 to 20 ring carbon atoms include the groups mentioned above as examples of the aryl group. A phenyl group, a naphthyl group, an anthranyl group, a phenanthryl group, a terphenyl group, and the like are preferable as the aryl group including 6 to 20 ring carbon atoms.

In the formula (B), Ar₃₀₄ to Ar₃₀₆ are a substituted or unsubstituted v-valent aryl group including 6 to 40 ring carbon atoms. v is an integer from 1 to 4, and preferably 1 or 2.

Examples of the aryl group including 6 to 40 ring carbon atoms included in the compound represented by the formula (B) include the groups mentioned above as examples of the aryl group. A naphthyl group, an anthranyl group, a chrysenyl group, and a pyrenyl group are preferable as the aryl group including 6 to 40 ring carbon atoms.

A compound represented by the following formula (25) is preferable as the fluoranthene compound.

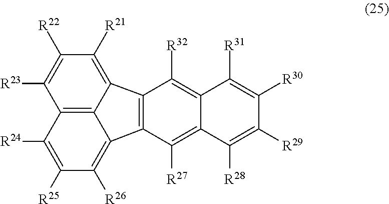

In the formula (25), R²¹ to R³² are independently selected from a hydrogen atom, a hydroxyl group, a cyano group, a nitro group, a carboxyl group, a substituted or unsubstituted silyl group, a substituted or unsubstituted alkyl group including 1 to 20 carbon atoms, a substituted or unsubstituted alkoxy group including 1 to 20 carbon atoms, a substituted or unsubstituted aralkyl group including 7 to 30 carbon atoms, a substituted or unsubstituted aryloxy group including 6 to 30 ring carbon atoms, a substituted or unsubstituted arylthio group including 6 to 30 ring carbon atoms, a substituted or unsubstituted alkoxycarbonyl group including 2 to 50 carbon atoms, a substituted or unsubstituted arylamino group including 6 to 30 ring carbon atoms, a substituted or unsubstituted aromatic hydrocarbon group including 6 to 30 ring carbon atoms, and a substituted or unsubstituted heterocyclic group including 5 to 30 ring atoms.

R²¹ and R²², R²² and R²³, R²⁵ and R²⁶, R²⁶ and R²⁷, R²⁷ and R²⁸, R²⁶ and R²⁹, R²⁹ and R³⁰, R³⁰ and R³¹, and R³¹ and R³² in the formula (25) are optionally bonded to each other to form a saturated or unsaturated ring. The saturated or unsaturated ring is either substituted or unsubstituted.

It is preferable that R²⁴ in the formula (25) be a hydrogen atom.

It is preferable that R²⁷ and R³² in the formula (25) be a substituted or unsubstituted aromatic hydrocarbon group including 6 to 30 ring carbon atoms. It is preferable that R²⁷ and R³² be a substituted or unsubstituted phenyl group.

It is also preferable that R²¹, R²², R²⁴ to R²⁶, and R²⁸ to R³¹ in the formula (25) be a hydrogen atom, and R²³, R²⁷, and R³² in the formula (25) be a substituted or unsubstituted aromatic hydrocarbon group including 6 to 30 ring carbon atoms.

It is preferable that R²¹, R²², R²⁴ to R²⁶, and R²⁸ to R³¹ in the formula (25) be a hydrogen atom, R²⁷ and R³² in the formula (25) be a substituted or unsubstituted aromatic hydrocarbon group including 6 to 30 ring carbon atoms, R²³ in the formula (25) be —Ar²¹—Ar²², and Ar²¹ and Ar²² be independently a substituted or unsubstituted aromatic hydrocarbon group including 6 to 30 ring carbon atoms.

In this case, it is preferable that Ar²¹ and Ar²² be an aromatic hydrocarbon group that is substituted with a cyano group, a dibenzofuranyl group, a dibenzothiophenyl group, or a carbazolyl group.

It is also preferable that R²¹, R²², R²⁴ to R²⁶, and R²⁸ to R³¹ in the formula (25) be a hydrogen atom, R²⁷ and R³² in the formula (25) be a substituted or unsubstituted aromatic hydrocarbon group including 6 to 30 ring carbon atoms, R²³ in the formula (25) be —Ar²¹—Ar²²—Ar²³, and Ar²¹, Ar²², and Ar²³ be independently a substituted or unsubstituted aromatic hydrocarbon group including 6 to 30 ring carbon atoms.

In this case, it is preferable that Ar²¹, Ar²², and Ar²³ be an aromatic hydrocarbon group that is substituted with a cyano group, a dibenzofuranyl group, a dibenzothiophenyl group, or a carbazolyl group.

Examples of a preferable substituent include an alkyl group including 1 to 6 carbon atoms, an alkoxy group including 1 to 6 carbon atoms, an aryl group including 6 to 40 ring carbon atoms, an amino group that is substituted with an aryl group including 6 to 40 ring carbon atoms, an ester group that includes an aryl group including 5 to 40 ring carbon atoms, an ester group that includes an alkyl group including 1 to 6 carbon atoms, a cyano group, a nitro group, a halogen atom, and the like.

The hole-injecting material is preferably a compound that has a capability to transport holes, exhibits an excellent hole-injecting effect with respect to the anode and the emitting layer or the emitting material, and exhibits an excellent thin film-forming capability. Specific examples of the hole-injecting material include, but are not limited to, a phthalocyanine derivative, a naphthalocyanine derivative, a porphyrin derivative, a benzidine-type triphenylamine, a diamine-type triphenylamine, hexacyanohexaazatriphenylene, derivatives thereof, and a polymer material such as polyvinylcarbazole, a polysilane, and a conductive polymer.

A phthalocyanine derivative is effective as the hole-injecting material that may be used for the organic EL device.

Examples of the phthalocyanine (Pc) derivative include, but are not limited to, a phthalocyanine derivative and a naphthalocyanine derivative such as H2Pc, CuPc, CoPc, NiPc, ZnPc, PdPc, FePc, MnPc, ClAlPc, ClGaPc, ClInPc, ClSnPc, Cl2SiPc, (HO)AlPc, (HO)GaPc, VOPc, TiOPc, MoOPc, and GaPc—O—GaPc.

It is possible to sensitize carriers by adding an electron-accepting substance (e.g., TCNQ derivative) to the hole-injecting material.

An aromatic tertiary amine derivative is preferable as the hole-transporting material that may be used for the organic EL device.

Examples of the aromatic tertiary amine derivative include, but are not limited to, N,N-diphenyl-N,N-dinaphthyl-1,1′-biphenyl-4,4′-diamine, N,N,N′,N′-tetrabiphenyl-1,1′-biphenyl-4,4′-diamine, and an oligomer or a polymer that includes such an aromatic tertiary amine skeleton.

The electron-injecting material is preferably a compound that has a capability to transport electrons, exhibits an excellent electron-injecting effect with respect to the cathode and the emitting layer or the emitting material, and exhibits an excellent thin film-forming capability.

A metal complex compound and a nitrogen-containing heterocyclic derivative are effective as the electron-injecting material that may be used for the organic EL device.

Examples of the metal complex compound include, but are not limited to, 8-hydroxyquinolinatolithium, bis(8-hydroxyquinolinato)zinc, tris(8-hydroxyquinolinato)aluminum, tris(8-hydroxyquinolinato)gallium, bis(10-hydroxybenzo[h]quinolinato)beryllium, bis(10-hydroxybenzo[h]quinolinato)zinc, and the like.

Examples of a preferable nitrogen-containing heterocyclic derivative include oxazole, thiazole, oxadiazole, thiadiazole, triazole, pyridine, pyrimidine, triazine, phenanthroline, benzimidazole, imidazopyridine, and the like. A benzimidazole derivative, a phenanthroline derivative, and an imidazopyridine derivative are particularly preferable as the nitrogen-containing heterocyclic derivative.

It is preferable that the electron-injecting material further include a dopant. It is more preferable that the electron-injecting material be doped with a dopant such as an alkali metal in the vicinity of the cathode-side interface of the organic layer in order to facilitate the reception of electrons from the cathode.

Examples of the dopant include a donor metal, a donor metal compound, and a donor metal complex. These reducing dopants may be used either alone or in combination.

The emitting layer included in the organic EL device may include at least one of the emitting material, the doping material, the hole-injecting material, the hole-transporting material, and the electron-injecting material in addition to at least one type of the anthracene derivative represented by the formula (1). A protective layer may be provided on the surface of the organic EL device, or the entire organic EL device may be protected with a silicone oil, a resin, or the like so that the resulting organic EL device exhibits improved stability against temperature, humidity, atmosphere, and the like.

A conductive material having a work function larger than 4 eV is suitable as the conductive material used to form the anode included in the organic EL device. Carbon, aluminum, vanadium, iron, cobalt, nickel, tungsten, silver, gold, platinum, palladium, an alloy thereof, a metal oxide such as tin oxide or indium oxide used for an ITO substrate or an NESA substrate, or an organic conductive resin such as polythiophene or polypyrrole may be used as the conductive material used to form the anode. A conductive material having a work function smaller than 4 eV is suitable as the conductive material used to form the cathode. Magnesium, calcium, tin, lead, titanium, yttrium, lithium, ruthenium, manganese, aluminum, lithium fluoride, or an alloy thereof may be used as the conductive material used to form the cathode. Note that the conductive material is not limited thereto. Examples of the alloy include, but are not limited to, a magnesium/silver alloy, a magnesium/indium alloy, a lithium/aluminum alloy, and the like. The alloy ratio is appropriately selected taking account of the temperature of the deposition source, the atmosphere, the degree of vacuum, and the like. The anode and the cathode may optionally include two or more layers.

It is desirable that at least one side of the organic EL device be sufficiently transparent within the emission wavelength region of the device so that the device can efficiently emit light. It is desirable that the substrate also be transparent. A transparent electrode is formed by deposition, sputtering, or the like using the above conductive material so that the transparent electrode has given translucency. It is desirable that the emitting-side electrode have a light transmittance equal to or higher than 10%. The substrate is not limited as long as the substrate exhibits mechanical strength and thermal strength, and has transparency. Examples of the substrate include a glass substrate and a transparent resin film.

Each layer of the organic EL device may be formed using a dry film-forming method such as a vacuum deposition method, a sputtering method, a plasma method, or an ion plating method, or a wet film-forming method such as a spin coating method, a dipping method, or a flow coating method. The thickness of each layer is not particularly limited as long as each layer has an appropriate thickness. If the thickness of each layer is too large, it may be necessary to apply a high voltage in order to obtain a constant optical output (i.e., deterioration in efficiency may occur). If the thickness of each layer is too small, pinholes or the like may occur, and sufficient luminance may not be obtained when an electric field is applied. The thickness of each layer is normally 5 nm to 10 μm, and preferably 10 nm to 0.2 μm.

When using a wet film-forming method, the material for forming each layer is dissolved or dispersed in an appropriate solvent (e.g., ethanol, chloroform, tetrahydrofuran, or dioxane), and a thin film is formed using the solution or dispersion. The solvent is not particularly limited.

An organic EL material-containing solution that includes the anthracene derivative (i.e., organic EL material) and a solvent may be suitable for the wet film-forming method.

An appropriate resin or an appropriate additive may be added to each organic thin film layer in order to improve the film-forming capability and prevent the occurrence of pinholes, for example.

The organic EL device may be used for various electronic devices. For example, the organic EL device may be used as a flat (planar) emitting device (e.g., a flat panel display used for a wall TV), a backlight used for a copier, a printer, or a liquid crystal display, a light source used for an instrument (meter), a signboard, a marker lamp (light), and the like. The compound according to the invention may also be used in the fields of an electrophotographic photoreceptor, a photoelectric conversion device, a solar cell, an image sensor, and the like in addition to the field of an organic EL device.

### EXAMPLES

### Synthesis Example 1

### Synthesis of Intermediate A

An intermediate A was synthesized according to the following scheme.

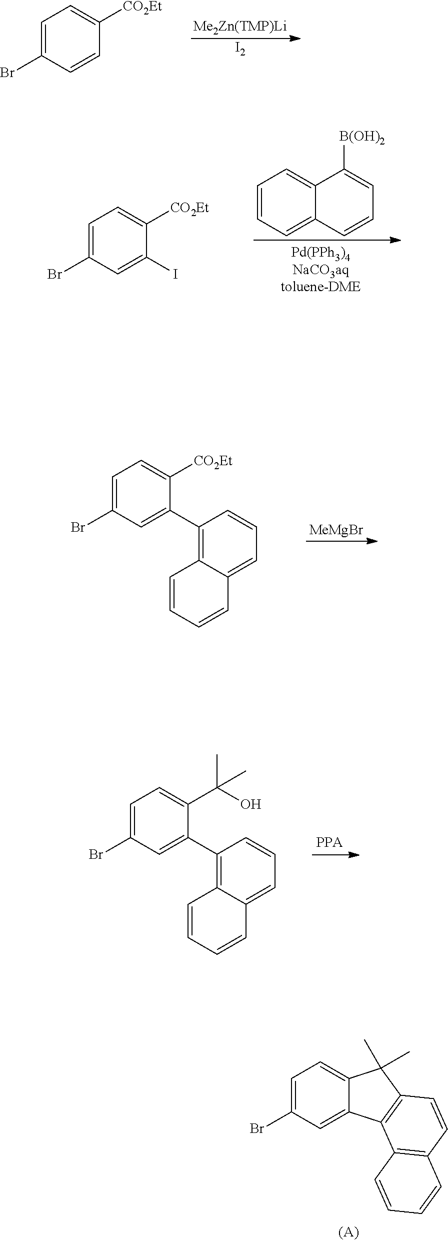

### (A-1) Synthesis of ethyl 4-bromo-2-iodobenzoate

36 mL of tetramethylpiperidine was added to 500 mL of tetrahydrofuran (THF) in an argon atmosphere. After cooling the mixture to 0° C., 90 mL of a 2.6 M hexane solution of n-BuLi was added dropwise to the mixture, and the resulting mixture was stirred at 0° C. for 10 minutes.

Separately, a 1.6 M pentane solution of n-BuLi was added dropwise to 440 mL of a THF solution (0.5 M) of zinc chloride at 0° C. in an argon atmosphere, and the mixture was stirred for 30 minutes. After cooling the tetramethylpiperidine solution to −78° C., the di-t-butylzinc solution prepared separately was added dropwise to the tetramethylpiperidine solution. The reaction solution was heated to 0° C., stirred for 30 minutes, and cooled to −78° C. After the dropwise addition of 22.9 g of ethyl 4-bromobenzoate to the reaction solution, the mixture was stirred for 3 hours while heating the mixture to 0° C. After the addition of a THF solution of 178 g of iodine to the reaction solution, the mixture was stirred at room temperature for 3 hours. After completion of the reaction, a saturated sodium thiosulfate solution and a saturated ammonium chloride solution were added to the reaction solution to effect quenching, followed by extraction with diethyl ether. The organic layer was washed with a saturated sodium chloride solution, and dried over magnesium sulfate. After removing the magnesium sulfate, the organic layer was concentrated, and the residue was purified by silica gel column chromatography to obtain 29.1 g of ethyl 4-bromo-2-iodobenzoate.

### (A-2) Synthesis of ethyl 4-bromo-2-(1-naphthyl)benzoate

A flask was charged with 15.5 g of 1-naphthaleneboronic acid, 29.1 g of ethyl 4-bromo-2-iodobenzoate, 1.89 g of tetrakis(triphenylphosphine)palladium(0), 220 mL of toluene, and 110 mL of a 2 M sodium carbonate aqueous solution in an argon atmosphere, and the mixture was refluxed for 8 hours with heating and stirring. The reaction solution was cooled to room temperature, and extracted with toluene. After removing the aqueous layer, the organic layer was washed with a saturated sodium chloride solution. The organic layer was dried over magnesium sulfate and concentrated, and the residue was purified by silica gel column chromatography to obtain 21.0 g of ethyl 4-bromo-2-(1-naphthyl)benzoate.

### (A-3) Synthesis of 2-[4-bromo-2-(1-naphthyl)phenyl]-2-propanol

100 mL of THF was added to 21.0 g of ethyl 4-bromo-2-(1-naphthyl)benzoate in an argon atmosphere, and the mixture was cooled to −30° C. 473 mL of a 1 M diethyl ether solution of methylmagnesium bromide was added dropwise to the mixture. The resulting mixture was stirred for 5 hours while heating the mixture to room temperature. 500 mL of a saturated ammonium chloride solution was slowly added to the mixture to effect quenching. The resulting mixture was extracted with ethyl acetate, and the aqueous layer was removed. The organic layer was washed with water, and dried over magnesium sulfate. After removing the magnesium sulfate, the organic layer was concentrated, and the residue was purified by silica gel column chromatography to obtain 13.1 g of 2-[4-bromo-2-(1-naphthyl)phenyl]-2-propanol.

### (A-4) Synthesis of 10-bromo-7,7-dimethylbenzo[c]fluorene

13.1 g of 2-[4-bromo-2-(1-naphthyl)phenyl]-2-propanol and 120 g of polyphosphoric acid were stirred at 100° C. for 5 hours in an argon atmosphere with heating. After cooling the reaction solution to room temperature, the reaction solution was slowly added to ice water. The resulting solid was filtered off, and purified by silica gel column chromatography to obtain 7.4 g of 10-bromo-7,7-dimethylbenzo[c]fluorene.

### Synthesis Example 2

### Synthesis of Intermediate B

An intermediate B was synthesized according to the following scheme.

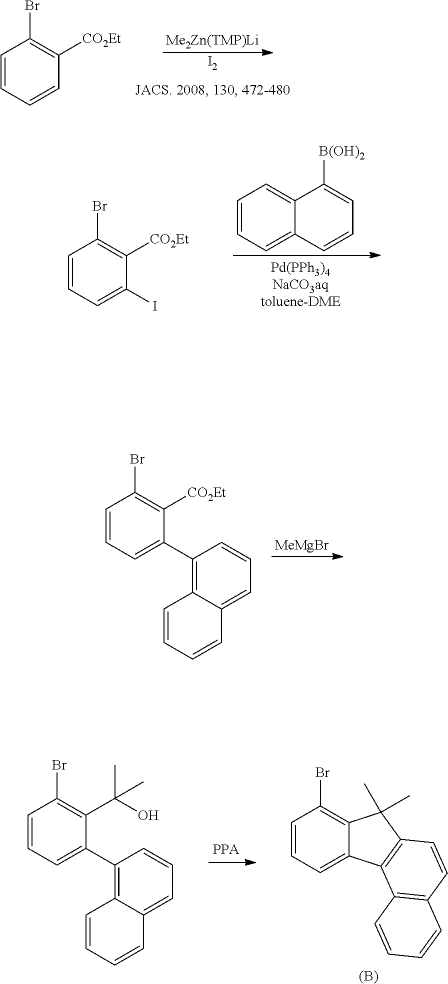

An intermediate B was synthesized in the same manner as the intermediate A, except that methyl 2-bromobenzoate was used instead of ethyl 4-bromobenzoate.

### Synthesis Example 3

### Synthesis of Intermediate C

An intermediate C was synthesized according to the following scheme.

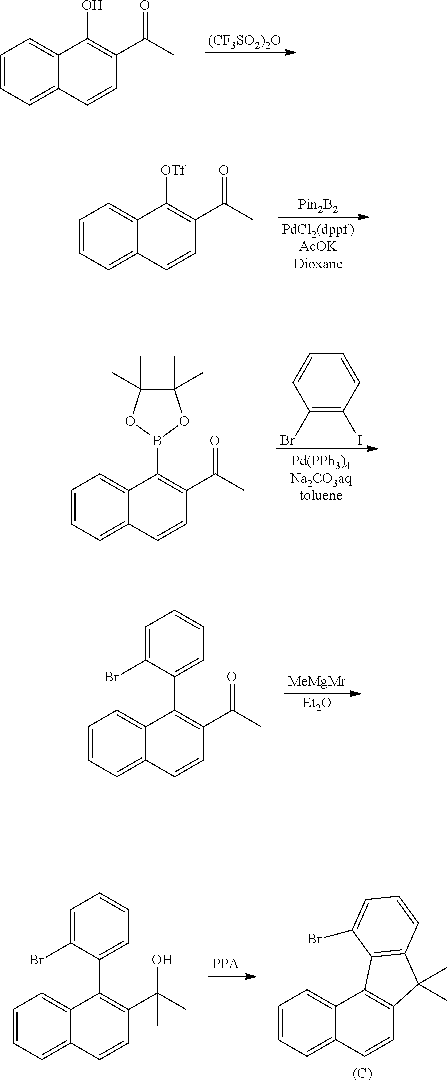

### (C-1) Synthesis of 2-acetyl-1-naphthyl trifluoromethanesulfonate

A flask was charged with 186 g of 1′-hydroxy-2′-acetonaphthone and 18.2 g of 4-dimethylaminopyridine in an argon atmosphere. After the addition of 4 L of methylene chloride, the mixture was cooled to −78° C. After the addition of 161 g of 2,6-dimethylpyridine, 339 g of trifluoromethanesulfonic anhydride was added dropwise to the mixture. The resulting mixture was stirred for 5 hours while heating the mixture to room temperature. A solid that precipitated was filtered off, washed with water and methanol, and dried to obtain 286 g (yield: 90%) of triphenylenyl trifluoromethanesulfonate.

### (C-2) Synthesis of 2-acetylnaphthalene-1-boronic acid pinacol ester

286 g of 2-acetyl-1-naphthyl trifluoromethanesulfonate, 251 g of bis(pinacolato)diboron, 22.0 g of [1,1-bis(diphenylphosphino)ferrocene]dichloropalladium(11), and 264 g of potassium acetate were mixed in an argon atmosphere. After the addition of 6 L of anhydrous dioxane, the mixture was refluxed for 8 hours with heating and stirring. After cooling the reaction solution to room temperature, 3 L of water was added to the reaction solution, followed by extraction with toluene. After removing the aqueous layer, the organic layer was washed with water and a saturated sodium chloride solution, and dried over magnesium sulfate. After removing the magnesium sulfate, the solvent was evaporated under reduced pressure. The residue was purified by silica gel column chromatography to obtain 160 g of 2-acetylnaphthalene-1-boronic acid pinacol ester.

### (C-3) Synthesis of 2-acetyl-1-(2-bromophenyl)naphthalene

160 g of 2-acetylnaphthalene-1-boronic acid pinacol ester, 153 g of 2-bromoiodobenzene, 12.5 g of tetrakis(triphenylphosphine)palladium(0), 2.2 L of toluene, and 1.1 L of a 2 M sodium carbonate aqueous solution were mixed in an argon atmosphere, and the mixture was refluxed for 8 hours with stirring. After cooling the reaction solution to room temperature, the reaction solution was extracted with toluene. After removing the aqueous layer, the organic layer was sequentially washed with water and a saturated sodium chloride solution, and dried over magnesium sulfate. After removing the magnesium sulfate by filtration, the organic layer was concentrated. The residue was purified by silica gel column chromatography to obtain 144 g of 2-acetyl-1-(2-bromophenyl)naphthalene.

### (C-4) Synthesis of 2-[1-(2-bromophenyl)naphthalen-2-yl]-2-propanol

2-[1-(2-Bromophenyl)naphthalen-2-yl]-2-propanol was synthesized in the same manner as in the step (A-3), except that 2-acetyl-1-(2-bromophenyl)naphthalene was used instead of ethyl 4-bromo-2-(1-naphthyl)benzoate.

### (C-5) Synthesis of Intermediate C

The intermediate C was synthesized in the same manner as in the step (A-4), except that 2-[1-(2-bromophenyl)naphthalen-2-yl]-2-propanol was used instead of 2-[4-bromo-2-(1-naphthyl)phenyl]-2-propanol.

### Synthesis Example 4

### Synthesis of Intermediate D

An intermediate D was synthesized according to the following scheme.

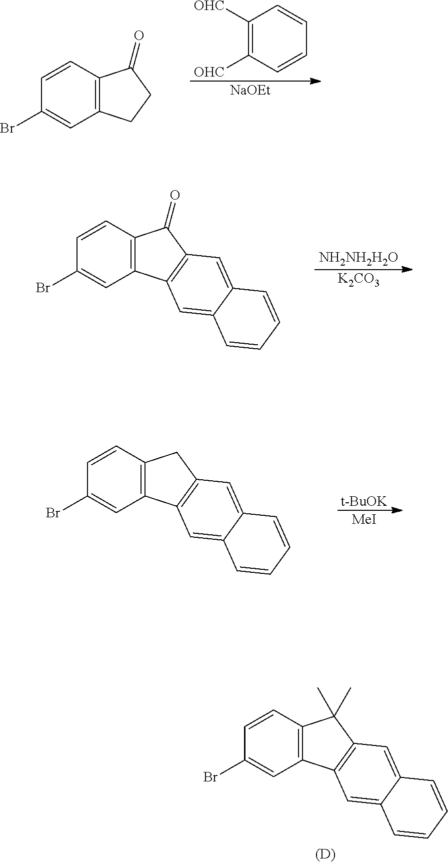

### (D-1) Synthesis of 3-bromobenzo[b]fluoren-11-one

211 g of 5-bromo-1-indanone, 134 g of o-phthalaldehyde, and 3 L of anhydrous ethanol were mixed in an argon atmosphere. After the addition of 78 mL of a 20 wt % ethanol solution of sodium ethoxide, the mixture was stirred at room temperature for 8 hours. The mixture was then refluxed for 24 hours with heating and stirring. After allowing the mixture to cool to room temperature, crystals that precipitated were filtered off. The resulting solid was recrystallized from ethanol to obtain 74.0 g of 3-bromo-11H-benzo[b]fluoren-11-one.

### (D-2) Synthesis of 3-bromo-11H-benzo[b]fluorene

74.0 g of 3-bromo-11H-benzo[b]fluoren-11-one, 300 mL of hydrazine monohydrate, 200 g of potassium carbonate, 700 mL of diethylene glycol, and 700 mL of chlorobenzene were mixed in an argon atmosphere, and the mixture was refluxed for 8 hours with heating and stirring. After cooling the mixture to room temperature, a 1 N hydrochloric acid aqueous solution was added to the mixture. The reaction solution was extracted with toluene, and the organic layer was washed with water and a saturated aqueous solution. The organic layer was dried over magnesium sulfate and concentrated, and the residue was purified by silica gel column chromatography to obtain 17.0 g of 3-bromo-11H-benzo[b]fluorene.

### (D-3) Synthesis of 3-bromo-11,11-dimethylbenzo[b]fluorene

17.0 g of 3-bromo-11H-benzo[b]fluorene, 15.5 g of potassium t-butoxide, and 250 mL of DMSO were mixed in an argon atmosphere. 19.6 g of methyl iodide was added dropwise to the reaction solution while stirring the reaction solution at 5° C. The reaction solution was stirred for 8 hours while heating the reaction solution to room temperature. After completion of the reaction, water was added to the reaction solution to effect quenching, followed by extraction with toluene. After removing the aqueous layer, the organic layer was washed with a saturated sodium chloride solution, and dried over magnesium sulfate. After removing the magnesium sulfate, the organic layer was concentrated, and the residue was purified by silica gel column chromatography to obtain 15.3 g of 3-bromo-11,11′-dimethyl-11H-benzo[b]fluorene.

### Synthesis Example 5

### Synthesis of Intermediate E

An intermediate E was synthesized according to the following scheme.

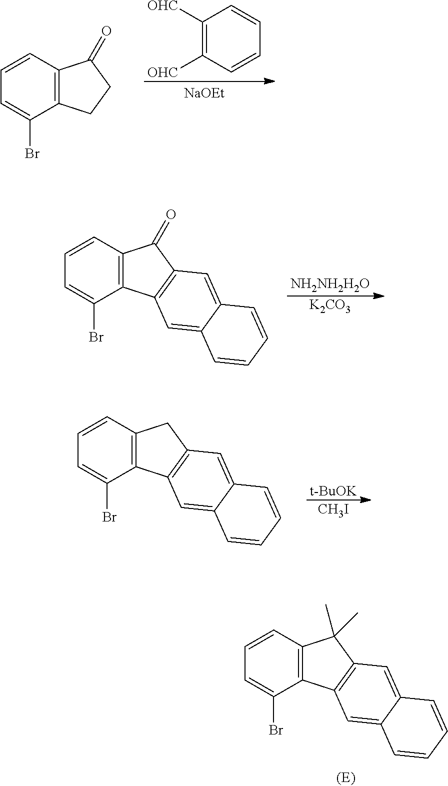

Specifically, the intermediate E was synthesized in the same manner as in Synthesis Example 4 (“Synthesis of intermediate D”), except that 4-bromo-1-indanone was used instead of 5-bromo-1-indanone.

### Synthesis Example 6

### Synthesis of Intermediate F

An intermediate F was synthesized according to the following scheme.

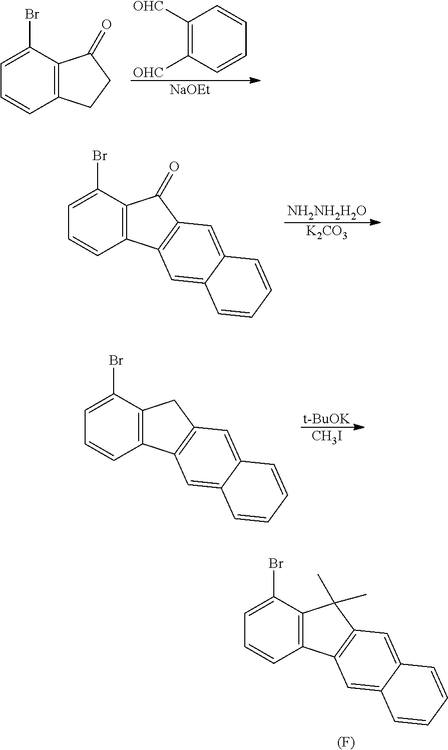

Specifically, the intermediate F was synthesized in the same manner as in Synthesis Example 4 (“Synthesis of intermediate D”), except that 7-bromo-1-indanone (synthesized using a known method) was used instead of 5-bromo-1-indanone.

### Synthesis Example 7

### Synthesis of Intermediate G

An intermediate G was synthesized according to the following scheme.

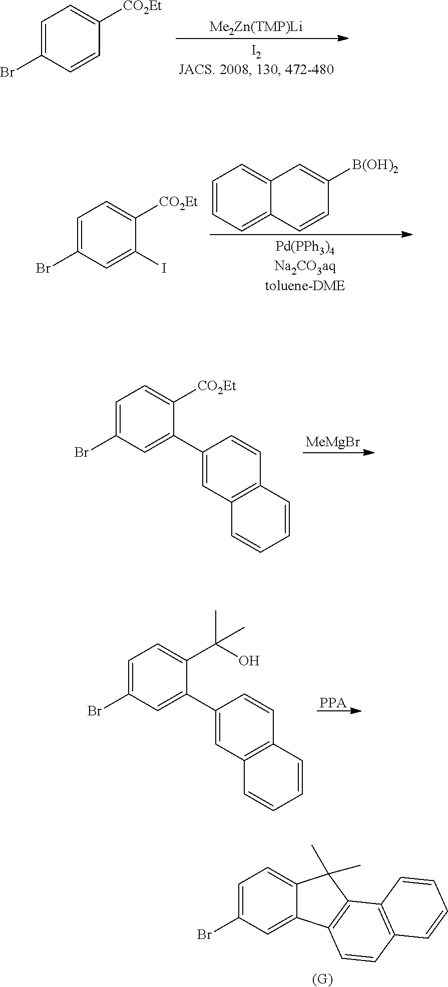

Specifically, the intermediate G was synthesized in the same manner as in Synthesis Example 1 (“Synthesis of intermediate A”), except that 2-naphthaleneboronic acid was used instead of 1-naphthaleneboronic acid.

### Synthesis Example 8

### Synthesis of Intermediate H

An intermediate H was synthesized according to the following scheme.

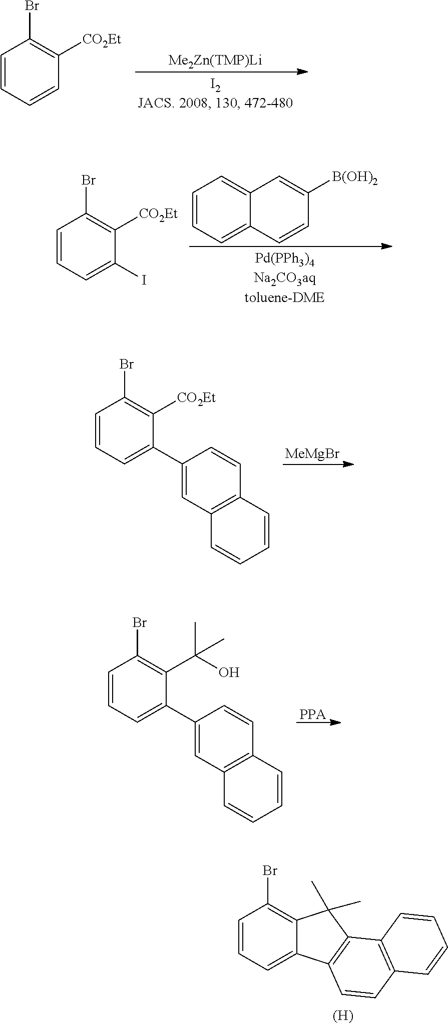

Specifically, the intermediate H was synthesized in the same manner as in Synthesis Example 2 (“Synthesis of intermediate B”), except that 2-naphthaleneboronic acid was used instead of 1-naphthaleneboronic acid.

### Synthesis Example 9

### Synthesis of Intermediate I

An intermediate I was synthesized according to the following scheme.

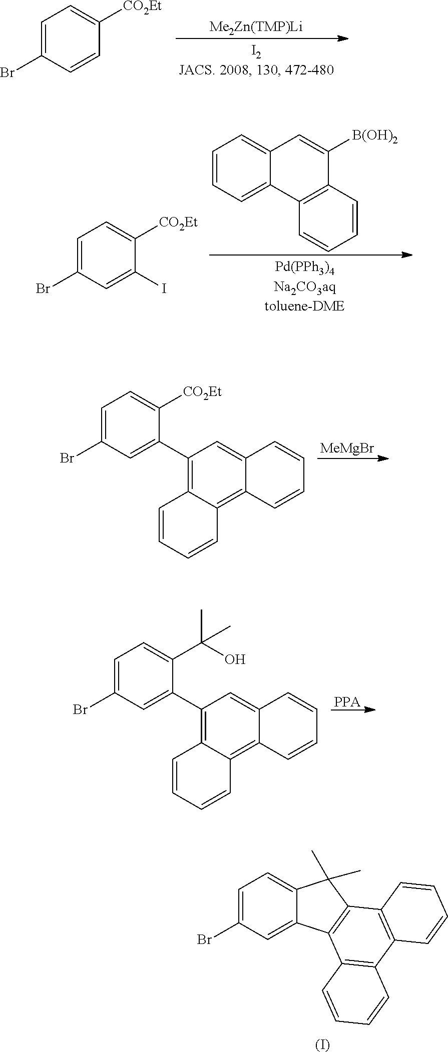

Specifically, the intermediate I was synthesized in the same manner as in Synthesis Example 1 (“Synthesis of intermediate A”), except that 9-phenanthreneboronic acid was used instead of 1-naphthaleneboronic acid.

### Synthesis Example 10

### Synthesis of Intermediate J

An intermediate J was synthesized according to the following scheme.

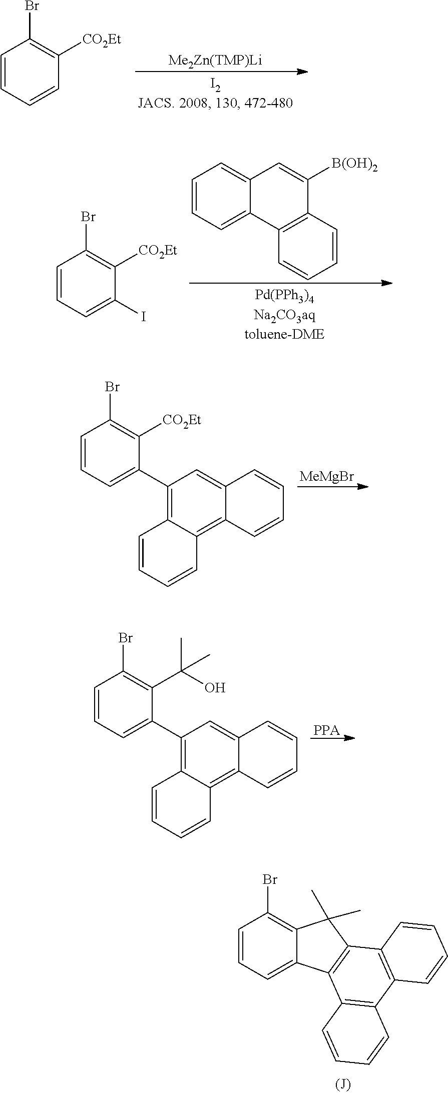

Specifically, the intermediate J was synthesized in the same manner as in Synthesis Example 2 (“Synthesis of intermediate B”), except that 9-phenanthreneboronic acid was used instead of 1-naphthaleneboronic acid.

### Example 1

### Synthesis of Compound 1-1

A compound 1-1 was synthesized according to the following scheme.

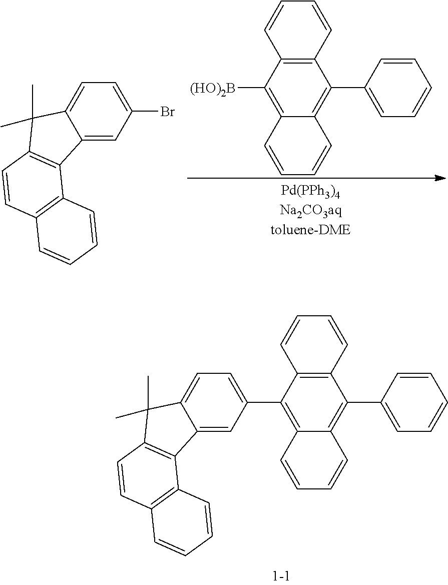

3.22 g of the intermediate A, 3.28 g of 10-phenylanthracene-9-boronic acid (synthesized using a known method), 0.231 g of tetrakis(triphenylphosphine)palladium(0), 20 mL of 1,2-dimethoxyethane, 20 mL of toluene, and 20 mL of a 2 M sodium carbonate aqueous solution were mixed in an argon atmosphere, and the mixture was refluxed for 8 hours with stirring. After cooling the mixture to room temperature, a solid that precipitated was filtered off. The solid was washed with water and methanol, and recrystallized from toluene to obtain 4.12 g of the compound 1-1 (light yellow solid). It was found by mass spectroscopy that the compound 1-1 was obtained. The compound 1-1 had a molecular weight of 496.22 (m/e=496).

### Example 2

### Synthesis of Compound 1-2

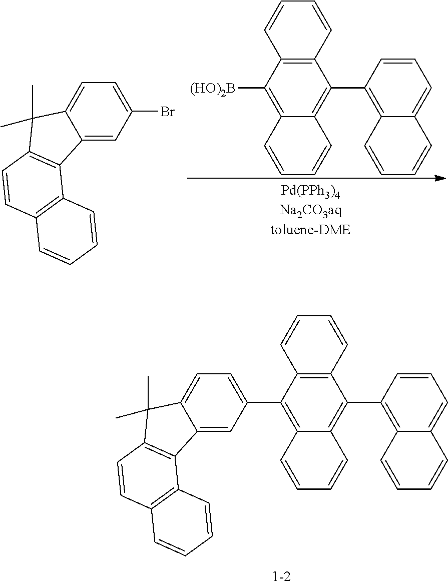

A compound 1-2 was synthesized in the same manner as in Example 1 according to the above scheme. It was found by mass spectroscopy that the compound 1-2 was obtained. The compound 1-2 had a molecular weight of 546.23 (m/e=546).

### Example 3

### Synthesis of Compound 1-3

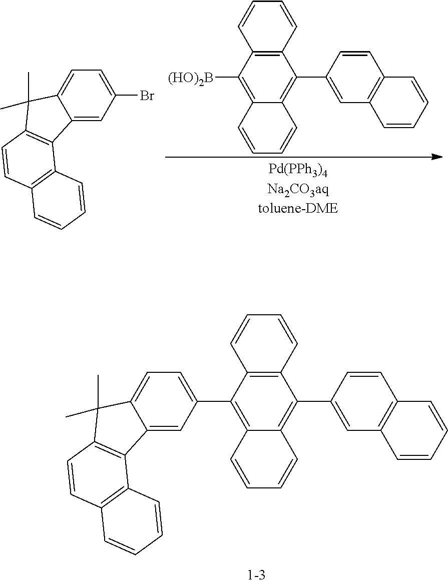

A compound 1-3 was synthesized in the same manner as in Example 1 according to the above scheme. It was found by mass spectroscopy that the compound 1-3 was obtained. The compound 1-3 had a molecular weight of 546.23 (m/e=546).

### Example 4

### Synthesis of Compound 1-4

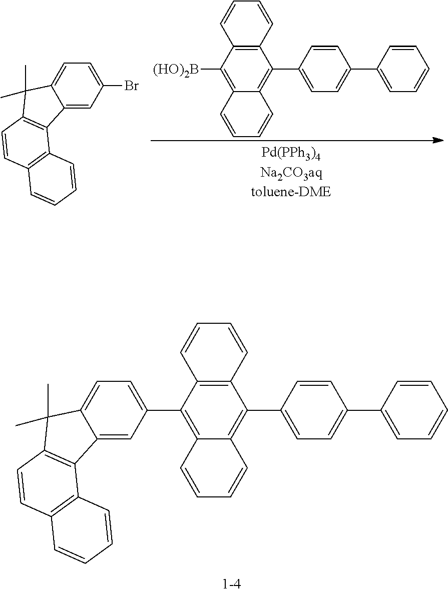

A compound 1-4 was synthesized in the same manner as in Example 1 according to the above scheme. It was found by mass spectroscopy that the compound 1-4 was obtained. The compound 1-4 had a molecular weight of 572.25 (m/e=572).

### Example 5

### Synthesis of Compound 1-5

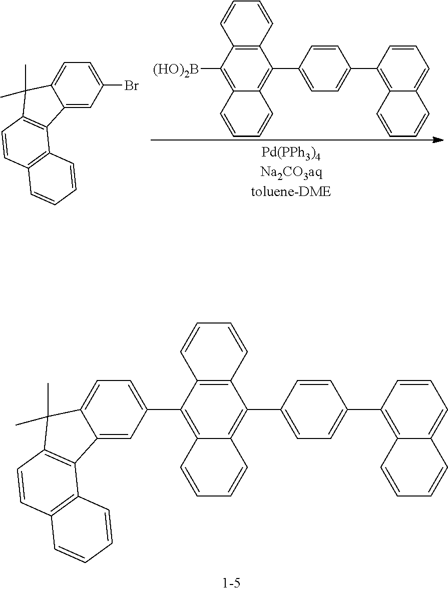

A compound 1-5 was synthesized in the same manner as in Example 1 according to the above scheme. It was found by mass spectroscopy that the compound 1-5 was obtained. The compound 1-5 had a molecular weight of 622.27 (m/e=622).

### Example 6

### Synthesis of Compound 1-6

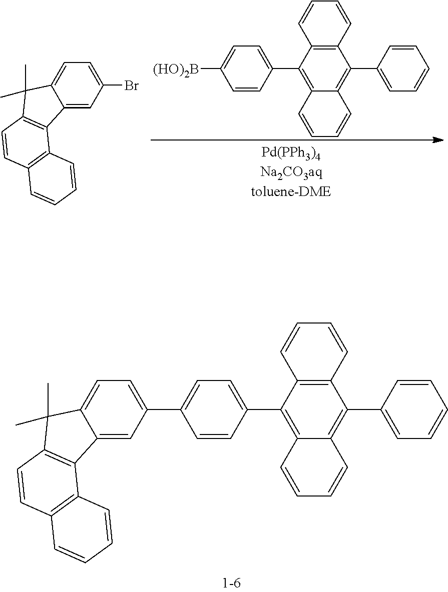

A compound 1-6 was synthesized in the same manner as in Example 1 according to the above scheme. It was found by mass spectroscopy that the compound 1-6 was obtained. The compound 1-6 had a molecular weight of 572.25 (m/e=572).

### Example 7

### Synthesis of Compound 1-7

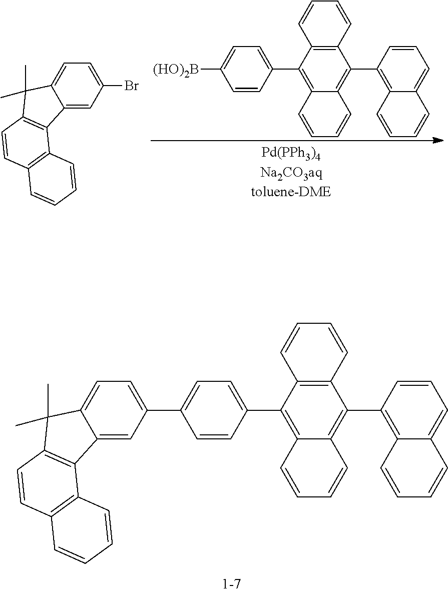

A compound 1-7 was synthesized in the same manner as in Example 1 according to the above scheme. It was found by mass spectroscopy that the compound 1-7 was obtained. The compound 1-7 had a molecular weight of 622.27 (m/e=622).

### Example 8

### Synthesis of Compound 1-8

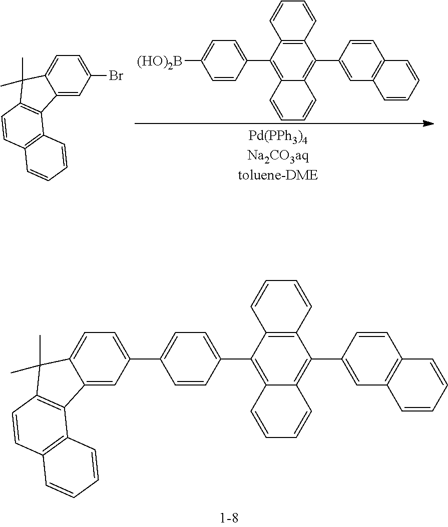

A compound 1-8 was synthesized in the same manner as in Example 1 according to the above scheme. It was found by mass spectroscopy that the compound 1-8 was obtained. The compound 1-8 had a molecular weight of 622.27 (m/e=622).

### Example 9

### Synthesis of Compound 1-9

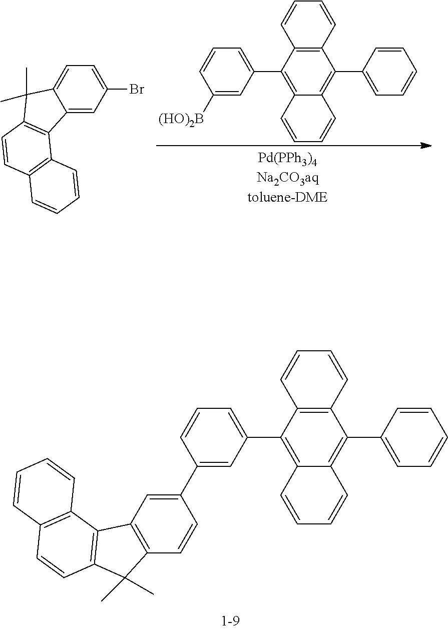

A compound 1-9 was synthesized in the same manner as in Example 1 according to the above scheme. It was found by mass spectroscopy that the compound 1-9 was obtained. The compound 1-9 had a molecular weight of 572.25 (m/e=572).

### Example 10

### Synthesis of Compound 1-10

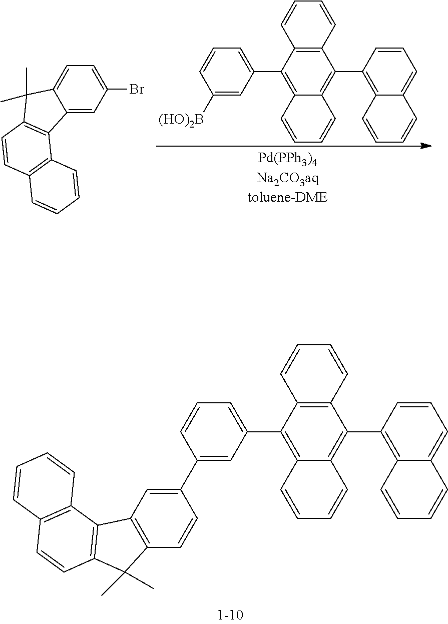

A compound 1-10 was synthesized in the same manner as in Example 1 according to the above scheme. It was found by mass spectroscopy that the compound 1-10 was obtained. The compound 1-10 had a molecular weight of 622.27 (m/e=622).

### Example 11

### Synthesis of Compound 1-11

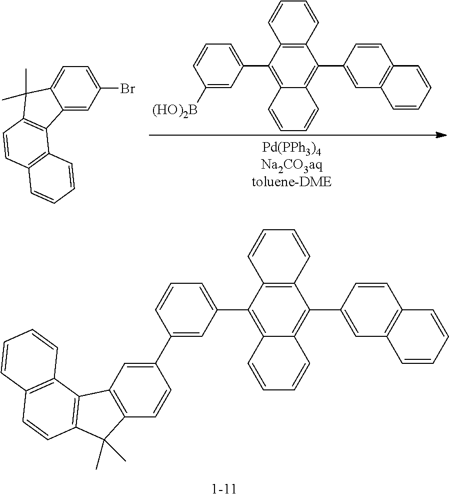

A compound 1-11 was synthesized in the same manner as in Example 1 according to the above scheme. It was found by mass spectroscopy that the compound 1-11 was obtained. The compound 1-11 had a molecular weight of 622.27 (m/e=622).

### Example 12

### Synthesis of Compound 1-12

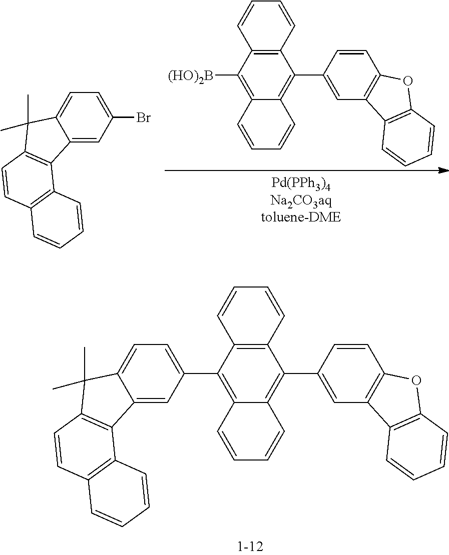 [SMILES: CC1(C)c2ccc(Br)cc2-c2c1ccc1ccccc21 ; OB(O)c1c2ccccc2c(-c2ccc3oc4ccccc4c3c2)c2ccccc12 ; CC1(C)c2ccc(-c3c4ccccc4c(-c4ccc5oc6ccccc6c5c4)c4ccccc34)cc2-c2c1ccc1ccccc21]

A compound 1-12 was synthesized in the same manner as in Example 1 according to the above scheme. It was found by mass spectroscopy that the compound 1-12 was obtained. The compound 1-12 had a molecular weight of 586.23 (m/e=586).

### Example 13

### Synthesis of Compound 1-13

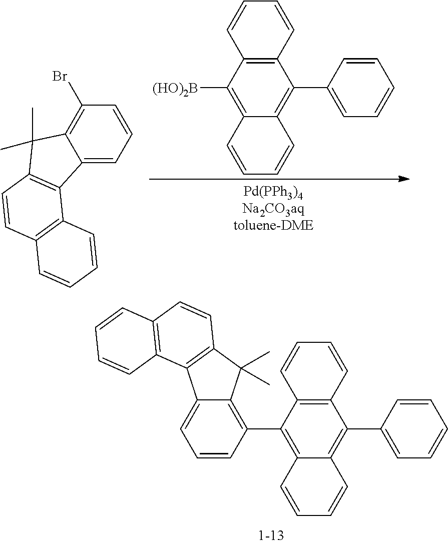

A compound 1-13 was synthesized in the same manner as in Example 1 according to the above scheme using the intermediate B. It was found by mass spectroscopy that the compound 1-13 was obtained. The compound 1-13 had a molecular weight of 496.22 (m/e=496).

### Example 14

### Synthesis of Compound 1-14

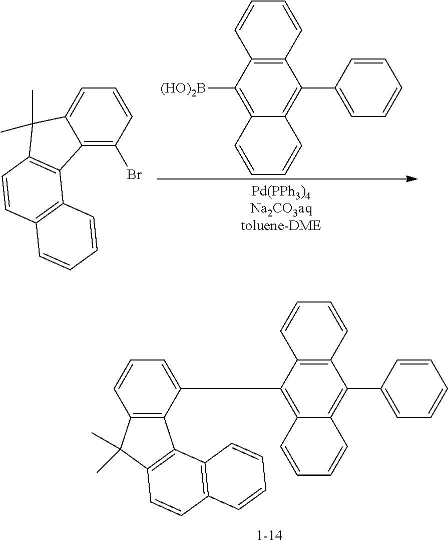

A compound 1-14 was synthesized in the same manner as in Example 1 according to the above scheme using the intermediate C. It was found by mass spectroscopy that the compound 1-14 was obtained. The compound 1-14 had a molecular weight of 496.22 (m/e=496).

### Example 15

### Synthesis of Compound 2-1

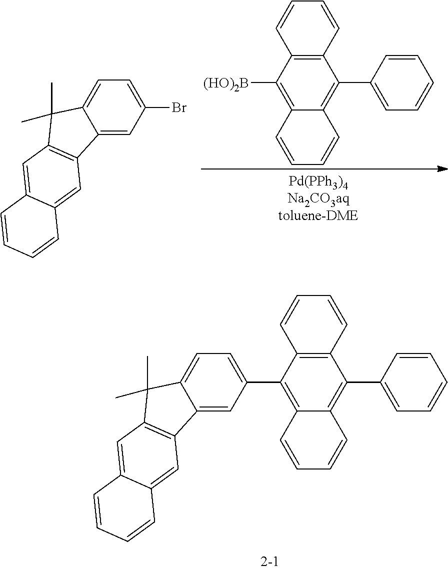

A compound 2-1 was synthesized in the same manner as in Example 1 according to the above scheme using the intermediate D. It was found by mass spectroscopy that the compound 2-1 was obtained. The compound 2-1 had a molecular weight of 496.22 (m/e=496).

### Example 16

### Synthesis of Compound 2-2

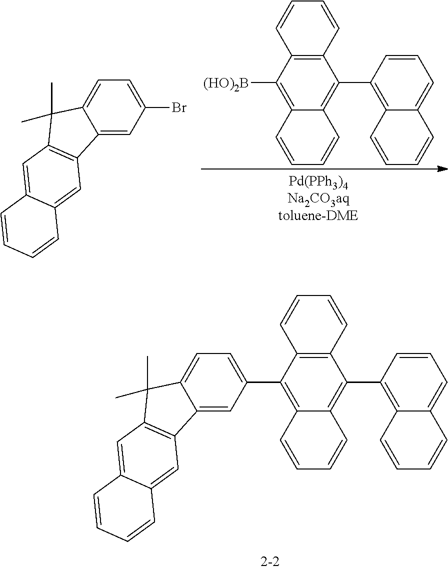

A compound 2-2 was synthesized in the same manner as in Example 1 according to the above scheme using the intermediate D. It was found by mass spectroscopy that the compound 2-2 was obtained. The compound 2-2 had a molecular weight of 546.23 (m/e=546).

### Example 17

### Synthesis of Compound 2-3

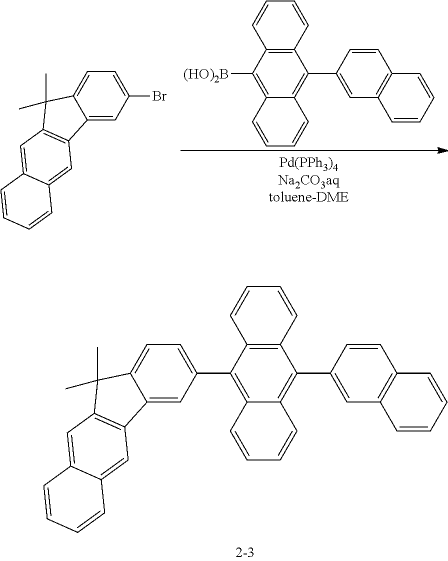

A compound 2-3 was synthesized in the same manner as in Example 1 according to the above scheme using the intermediate D. It was found by mass spectroscopy that the compound 2-3 was obtained. The compound 2-3 had a molecular weight of 546.23 (m/e=546).

### Example 18

### Synthesis of Compound 2-4

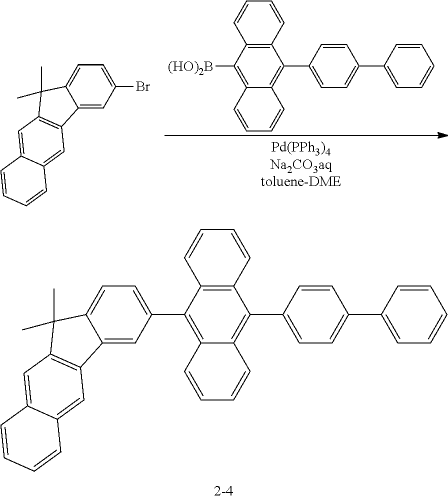

A compound 2-4 was synthesized in the same manner as in Example 1 according to the above scheme using the intermediate D. It was found by mass spectroscopy that the compound 2-4 was obtained. The compound 2-4 had a molecular weight of 572.25 (m/e=572).

### Example 19

### Synthesis of Compound 2-5

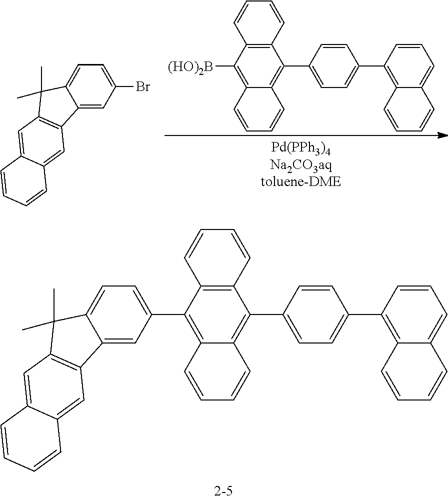

A compound 2-5 was synthesized in the same manner as in Example 1 according to the above scheme using the intermediate D. It was found by mass spectroscopy that the compound 2-5 was obtained. The compound 2-5 had a molecular weight of 622.27 (m/e=622).

### Example 20

### Synthesis of Compound 2-6

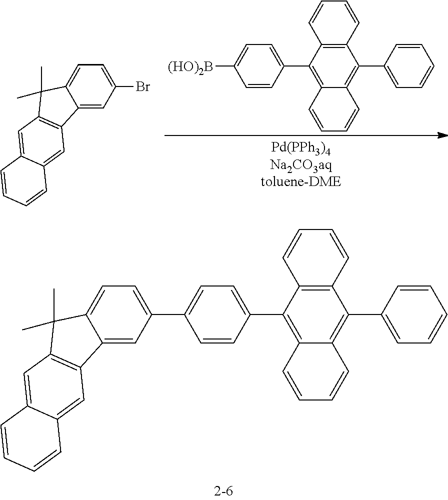

A compound 2-6 was synthesized in the same manner as in Example 1 according to the above scheme using the intermediate D. It was found by mass spectroscopy that the compound 2-6 was obtained. The compound 2-6 had a molecular weight of 572.25 (m/e=572).

### Example 21

### Synthesis of Compound 2-7

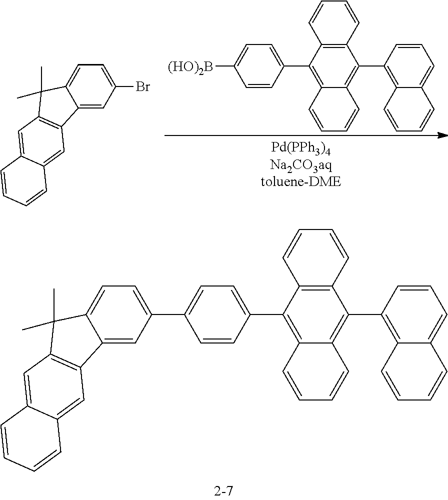

A compound 2-7 was synthesized in the same manner as in Example 1 according to the above scheme using the intermediate D. It was found by mass spectroscopy that the compound 2-7 was obtained. The compound 2-7 had a molecular weight of 622.27 (m/e=622).

### Example 22

### Synthesis of Compound 2-8

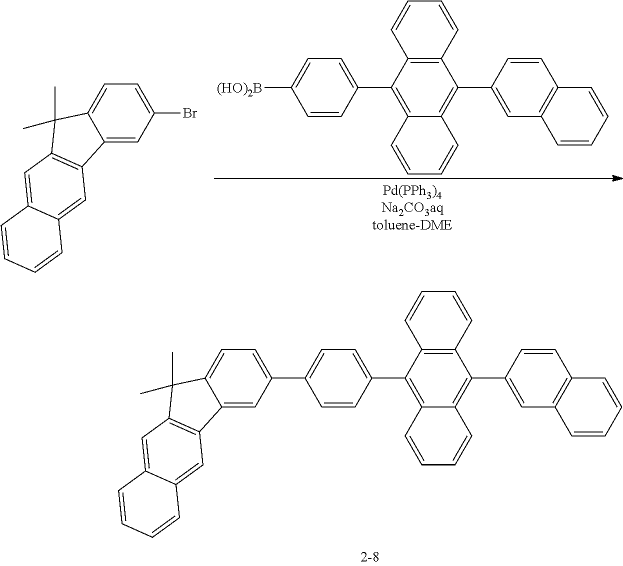

A compound 2-8 was synthesized in the same manner as in Example 1 according to the above scheme using the intermediate D. It was found by mass spectroscopy that the compound 2-8 was obtained. The compound 2-8 had a molecular weight of 622.27 (m/e=622).

### Example 23

### Synthesis of Compound 2-9

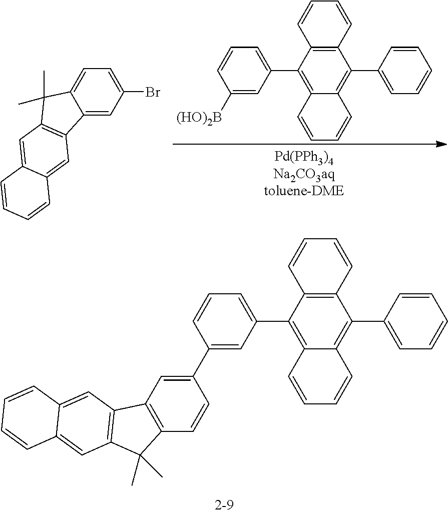

A compound 2-9 was synthesized in the same manner as in Example 1 according to the above scheme using the intermediate D. It was found by mass spectroscopy that the compound 2-9 was obtained. The compound 2-9 had a molecular weight of 572.25 (m/e=572).

### Example 24

### Synthesis of Compound 2-10

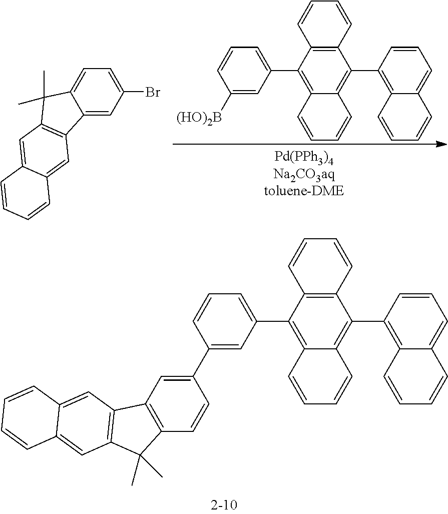

A compound 2-10 was synthesized in the same manner as in Example 1 according to the above scheme using the intermediate D. It was found by mass spectroscopy that the compound 2-10 was obtained. The compound 2-10 had a molecular weight of 622.27 (m/e=622).

### Example 25

### Synthesis of Compound 2-11

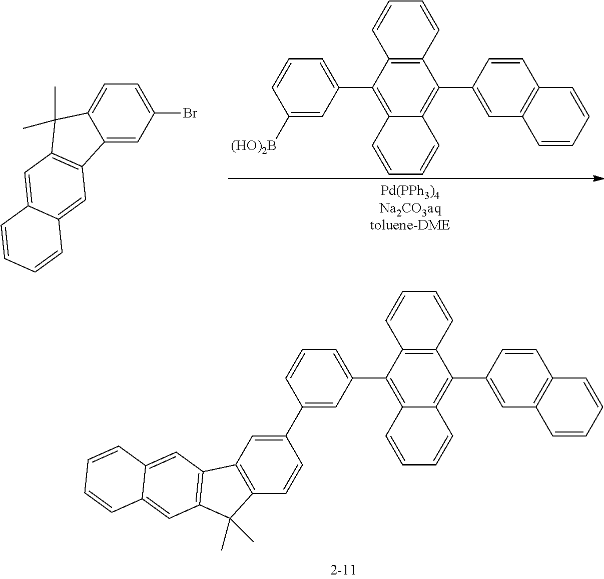

A compound 2-11 was synthesized in the same manner as in Example 1 according to the above scheme using the intermediate D. It was found by mass spectroscopy that the compound 2-11 was obtained. The compound 2-11 had a molecular weight of 622.27 (m/e=622).

### Example 26

### Synthesis of Compound 2-12

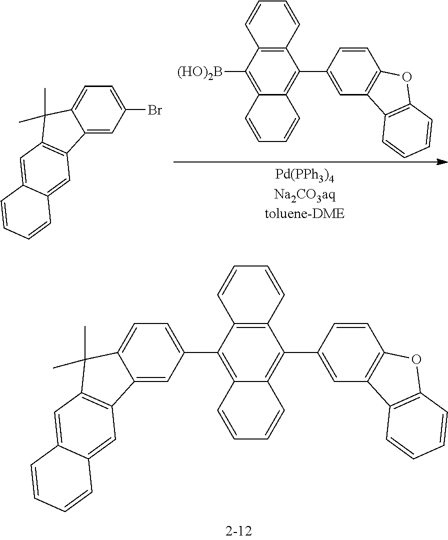 [SMILES: CC1(C)c2ccc(Br)cc2-c2cc3ccccc3cc21 ; OB(O)c1c2ccccc2c(-c2ccc3oc4ccccc4c3c2)c2ccccc12 ; CC1(C)c2ccc(-c3c4ccccc4c(-c4ccc5oc6ccccc6c5c4)c4ccccc34)cc2-c2cc3ccccc3cc21]

A compound 2-12 was synthesized in the same manner as in Example 1 according to the above scheme using the intermediate D. It was found by mass spectroscopy that the compound 2-12 was obtained. The compound 2-12 had a molecular weight of 586.23 (m/e=586).

### Example 27

### Synthesis of Compound 2-13

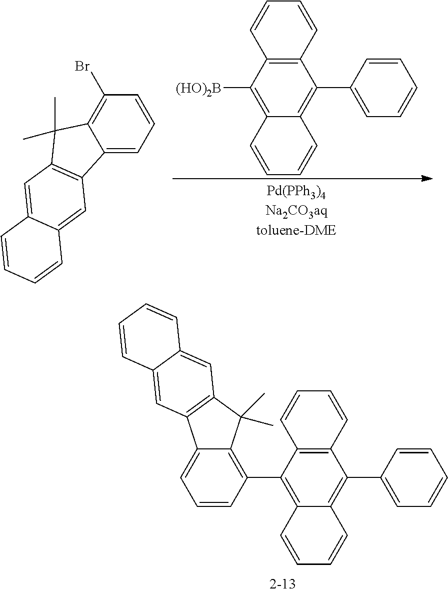

A compound 2-13 was synthesized in the same manner as in Example 1 according to the above scheme using the intermediate F. It was found by mass spectroscopy that the compound 2-13 was obtained. The compound 2-13 had a molecular weight of 496.22 (m/e=496).

### Example 28

### Synthesis of Compound 2-14

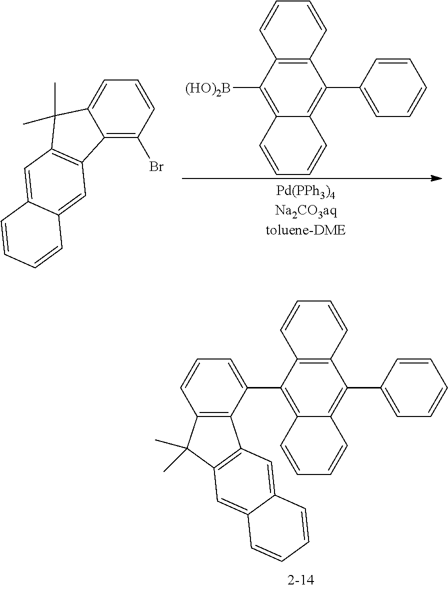

A compound 2-14 was synthesized in the same manner as in Example 1 according to the above scheme using the intermediate E. It was found by mass spectroscopy that the compound 2-14 was obtained. The compound 2-14 had a molecular weight of 496.22 (m/e=496).

### Example 29

### Synthesis of Compound 3-1

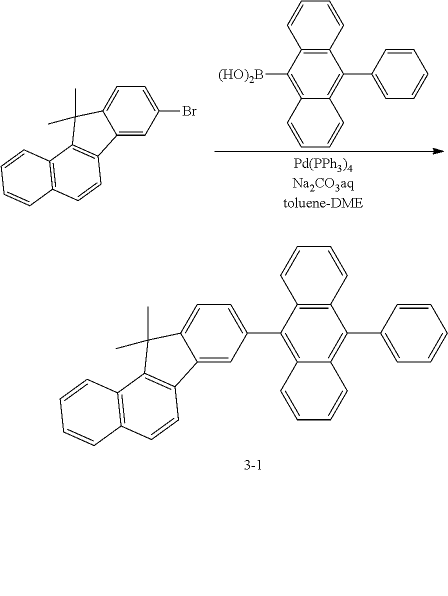

A compound 3-1 was synthesized in the same manner as in Example 1 according to the above scheme using the intermediate G. It was found by mass spectroscopy that the compound 3-1 was obtained. The compound 3-1 had a molecular weight of 496.22 (m/e=496).

### Example 30

### Synthesis of Compound 3-2

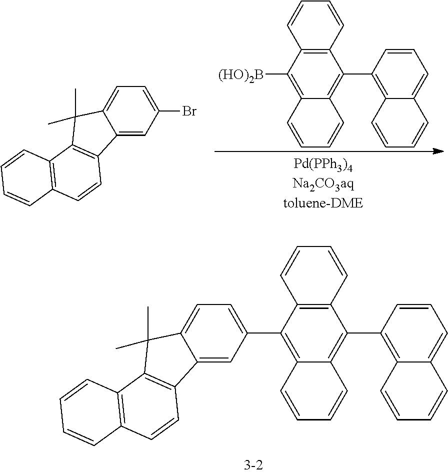

A compound 3-2 was synthesized in the same manner as in Example 1 according to the above scheme using the intermediate G It was found by mass spectroscopy that the compound 3-2 was obtained. The compound 3-2 had a molecular weight of 546.23 (m/e=546).

### Example 31

### Synthesis of Compound 3-3

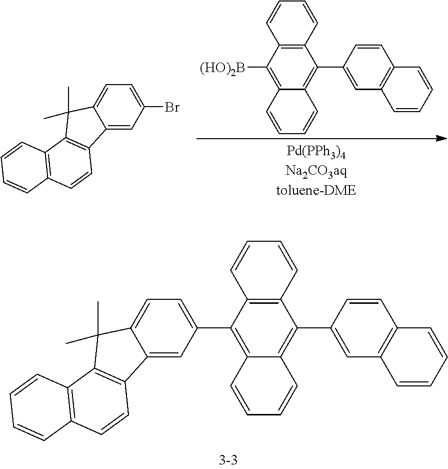

A compound 3-3 was synthesized in the same manner as in Example 1 according to the above scheme using the intermediate G. It was found by mass spectroscopy that the compound 3-3 was obtained. The compound 3-3 had a molecular weight of 546.23 (m/e=546).

### Example 32

### Synthesis of Compound 3-4

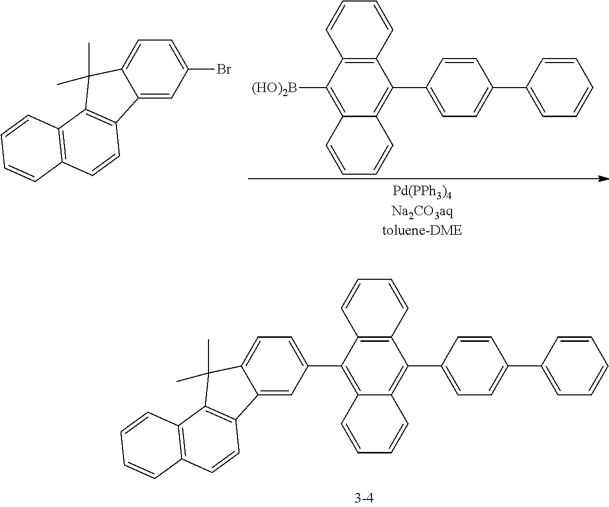

A compound 3-4 was synthesized in the same manner as in Example 1 according to the above scheme using the intermediate G. It was found by mass spectroscopy that the compound 3-4 was obtained. The compound 3-4 had a molecular weight of 572.25 (m/e=572).

### Example 33

### Synthesis of Compound 3-5

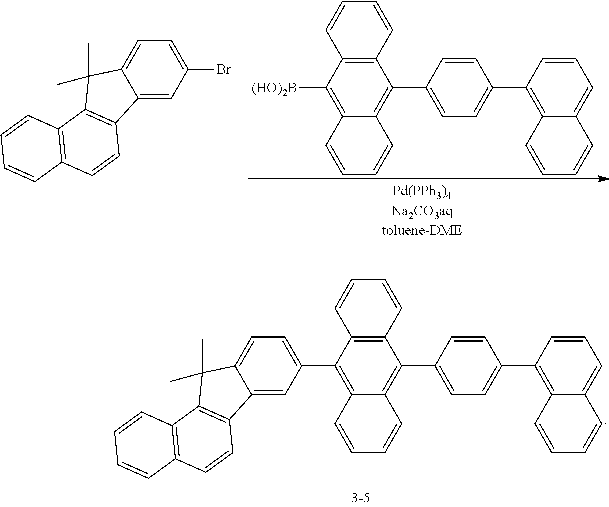

A compound 3-5 was synthesized in the same manner as in Example 1 according to the above scheme using the intermediate G. It was found by mass spectroscopy that the compound 3-5 was obtained. The compound 3-5 had a molecular weight of 622.27 (m/e=622).

### Example 34

### Synthesis of Compound 3-6

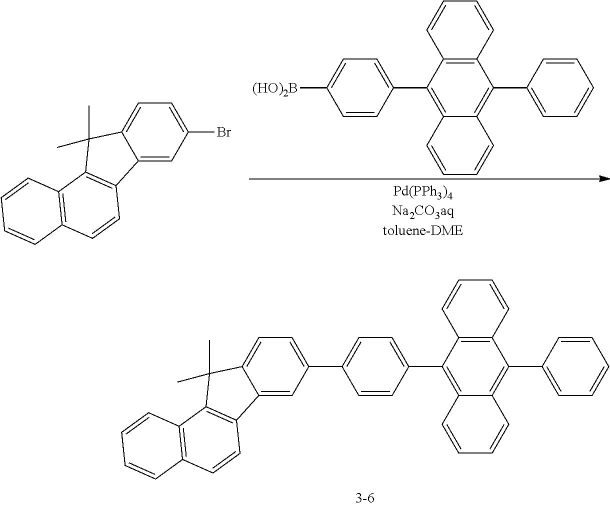

A compound 3-6 was synthesized in the same manner as in Example 1 according to the above scheme using the intermediate G It was found by mass spectroscopy that the compound 3-6 was obtained. The compound 3-6 had a molecular weight of 572.25 (m/e=572).

### Example 35

### Synthesis of Compound 3-7

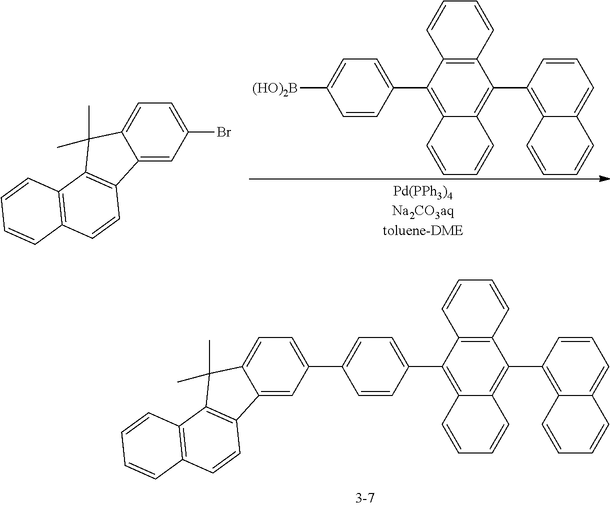

A compound 3-7 was synthesized in the same manner as in Example 1 according to the above scheme using the intermediate G. It was found by mass spectroscopy that the compound 3-7 was obtained. The compound 3-7 had a molecular weight of 622.27 (m/e=622).

### Example 36

### Synthesis of Compound 3-8

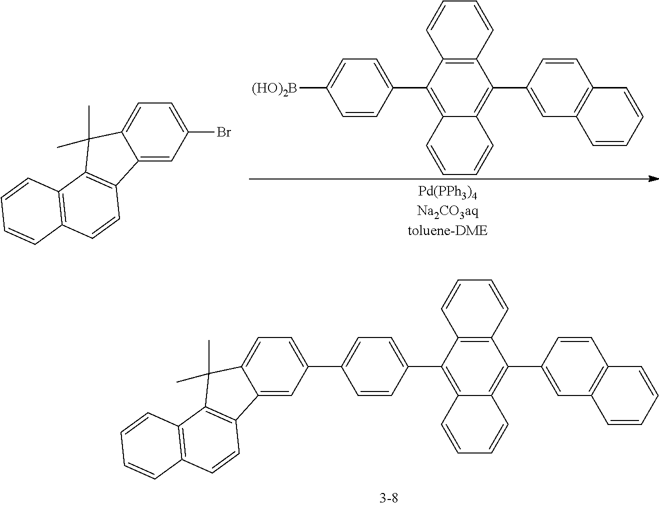

A compound 3-8 was synthesized in the same manner as in Example 1 according to the above scheme using the intermediate G It was found by mass spectroscopy that the compound 3-8 was obtained. The compound 3-8 had a molecular weight of 622.27 (m/e=622).

### Example 37

### Synthesis of Compound 3-9

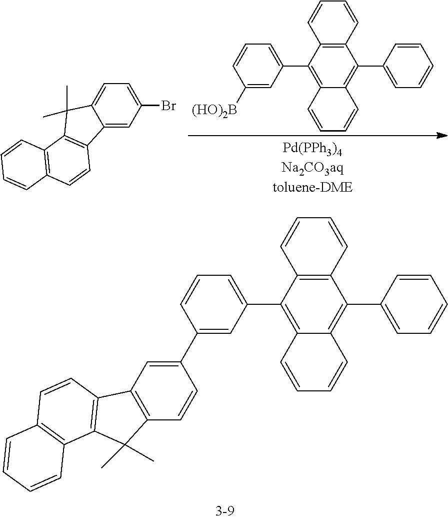

A compound 3-9 was synthesized in the same manner as in Example 1 according to the above scheme using the intermediate G. It was found by mass spectroscopy that the compound 3-9 was obtained. The compound 3-9 had a molecular weight of 572.25 (m/e=572).

### Example 38

### Synthesis of Compound 3-10

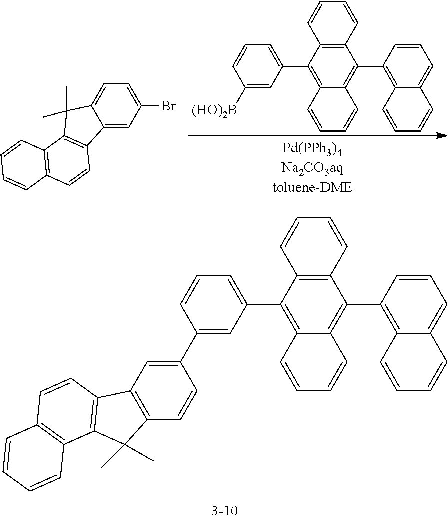

A compound 3-10 was synthesized in the same manner as in Example 1 according to the above scheme using the intermediate G. It was found by mass spectroscopy that the compound 3-10 was obtained. The compound 3-10 had a molecular weight of 622.27 (m/e=622).

### Example 39

### Synthesis of Compound 3-11

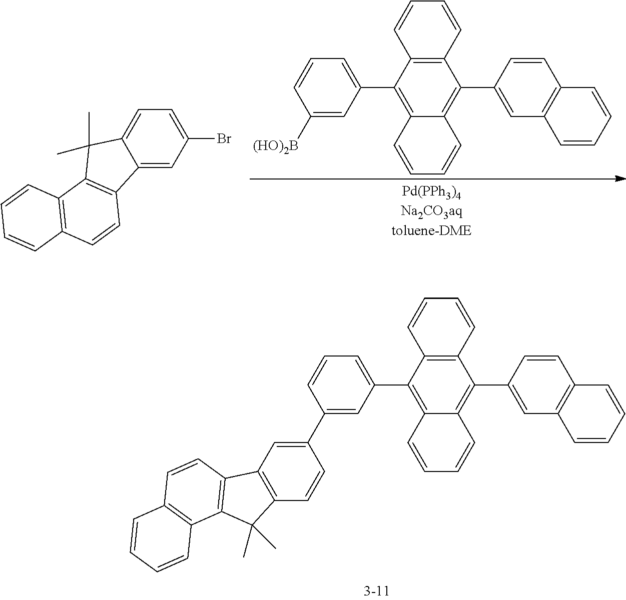

A compound 3-11 was synthesized in the same manner as in Example 1 according to the above scheme using the intermediate G. It was found by mass spectroscopy that the compound 3-11 was obtained. The compound 3-11 had a molecular weight of 622.27 (m/e=622).

### Example 40

### Synthesis of Compound 3-12

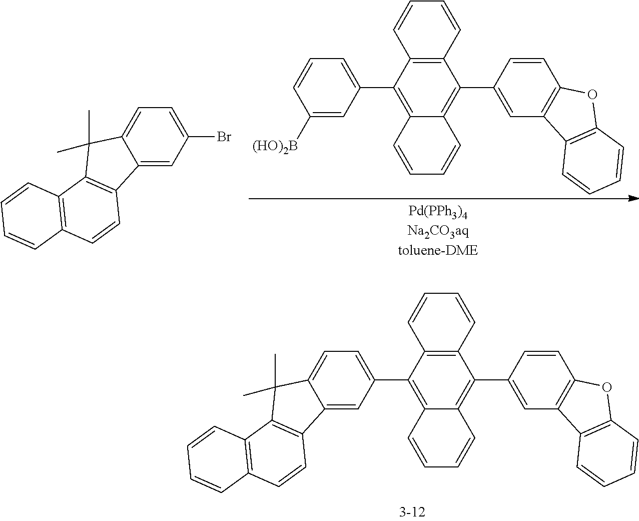 [SMILES: CC1(C)c2ccc(Br)cc2-c2ccc3ccccc3c21 ; OB(O)c1cccc(-c2c3ccccc3c(-c3ccc4oc5ccccc5c4c3)c3ccccc23)c1 ; CC1(C)c2ccc(-c3c4ccccc4c(-c4ccc5oc6ccccc6c5c4)c4ccccc34)cc2-c2ccc3ccccc3c21]

A compound 3-12 was synthesized in the same manner as in Example 1 according to the above scheme using the intermediate G. It was found by mass spectroscopy that the compound 3-12 was obtained. The compound 3-12 had a molecular weight of 586.23 (m/e=586).

### Example 41

### Synthesis of Compound 3-13

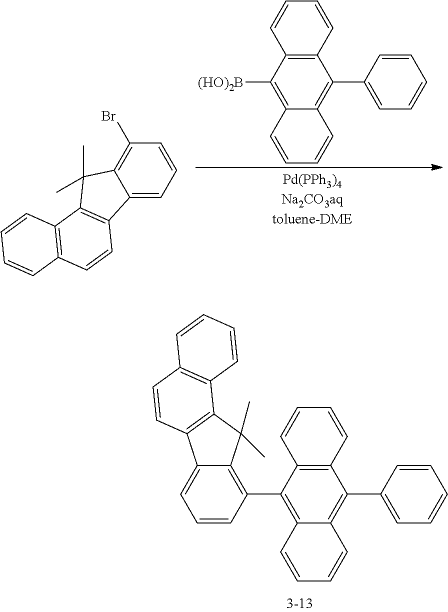

A compound 3-13 was synthesized in the same manner as in Example 1 according to the above scheme using the intermediate H. It was found by mass spectroscopy that the compound 3-13 was obtained. The compound 3-13 had a molecular weight of 496.22 (m/e=496).

### Example 42

### Synthesis of Compound 4-1

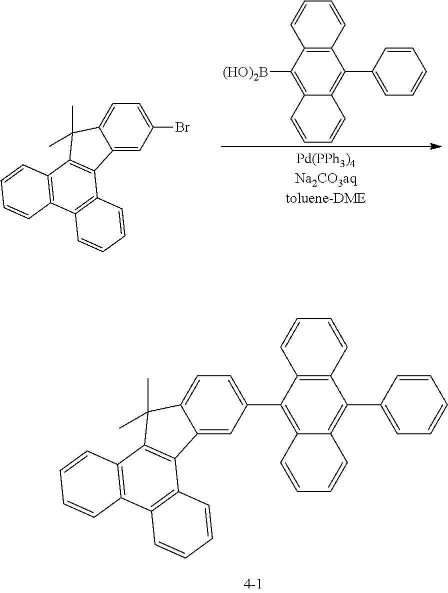

A compound 4-1 was synthesized in the same manner as in Example 1 according to the above scheme using the intermediate I. It was found by mass spectroscopy that the compound 4-1 was obtained. The compound 4-1 had a molecular weight of 546.23 (m/e=546).

### Example 43

### Synthesis of Compound 4-2

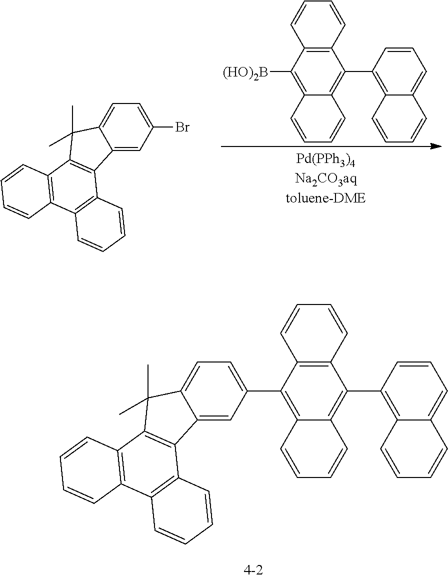

A compound 4-2 was synthesized in the same manner as in Example 1 according to the above scheme using the intermediate I. It was found by mass spectroscopy that the compound 4-2 was obtained. The compound 4-2 had a molecular weight of 596.25 (m/e=596).

### Example 44

### Synthesis of Compound 4-3

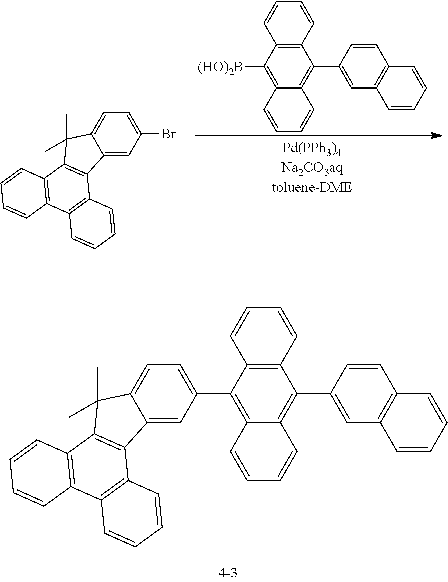

A compound 4-3 was synthesized in the same manner as in Example 1 according to the above scheme using the intermediate I. It was found by mass spectroscopy that the compound 4-3 was obtained. The compound 4-3 had a molecular weight of 596.25 (m/e=596).

### Example 45

### Synthesis of Compound 4-4

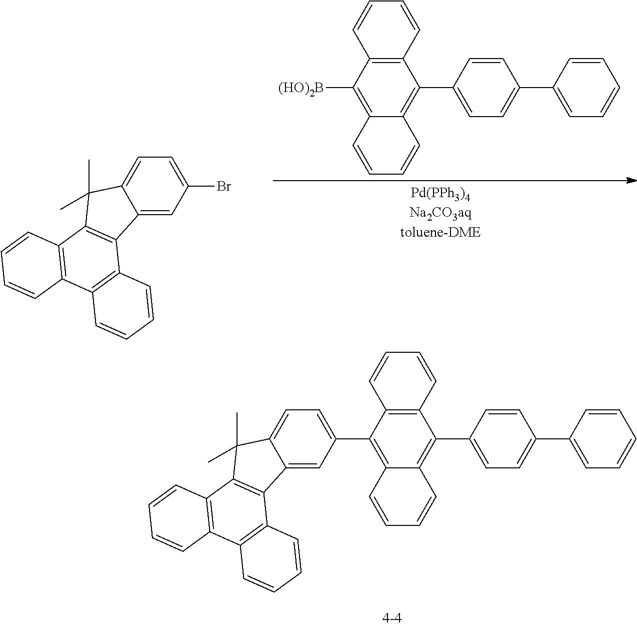

A compound 4-4 was synthesized in the same manner as in Example 1 according to the above scheme using the intermediate I. It was found by mass spectroscopy that the compound 4-4 was obtained. The compound 4-4 had a molecular weight of 622.27 (m/e=622).

### Example 46

### Synthesis of Compound 4-5

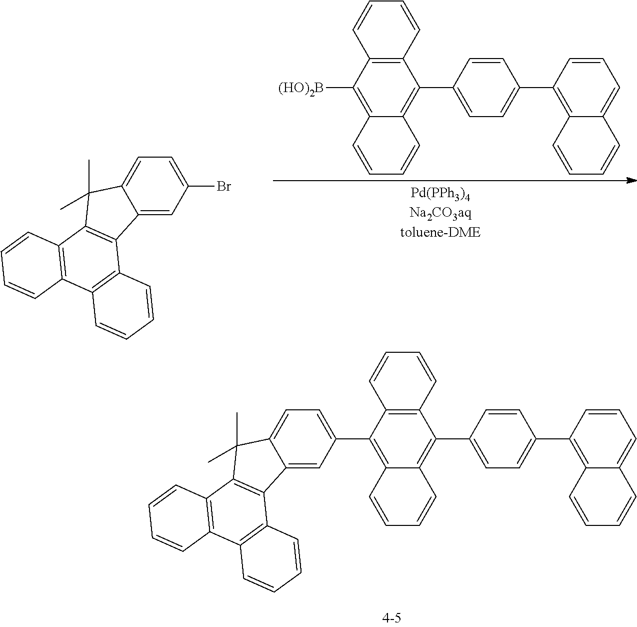

A compound 4-5 was synthesized in the same manner as in Example 1 according to the above scheme using the intermediate I. It was found by mass spectroscopy that the compound 4-5 was obtained. The compound 4-5 had a molecular weight of 672.28 (m/e=672).

### Example 47

### Synthesis of Compound 4-6

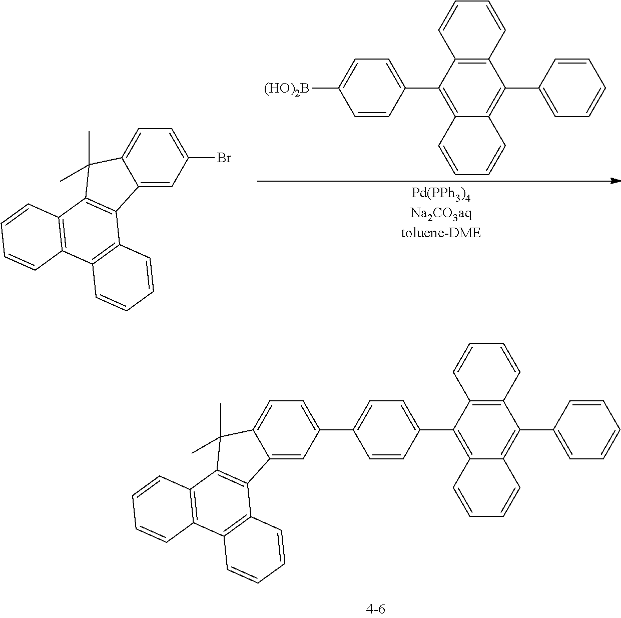

A compound 4-6 was synthesized in the same manner as in Example 1 according to the above scheme using the intermediate I. It was found by mass spectroscopy that the compound 4-6 was obtained. The compound 4-6 had a molecular weight of 622.27 (m/e=622).

### Example 48

### Synthesis of Compound 4-7

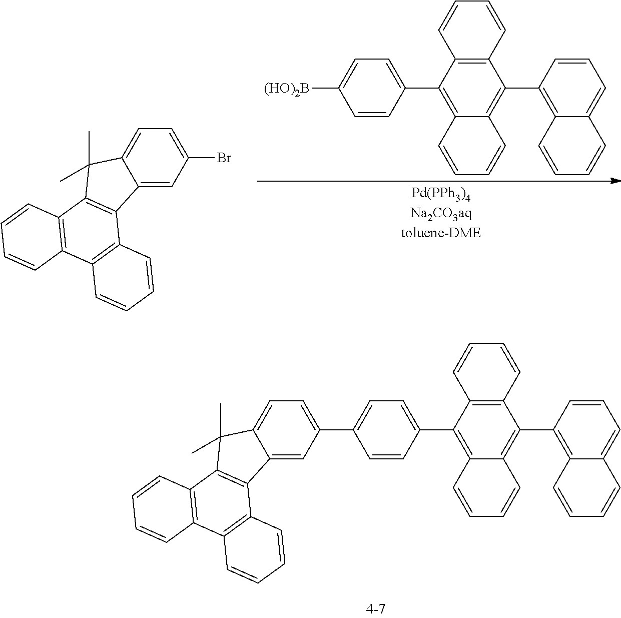

A compound 4-7 was synthesized in the same manner as in Example 1 according to the above scheme using the intermediate I. It was found by mass spectroscopy that the compound 4-7 was obtained. The compound 4-7 had a molecular weight of 672.28 (m/e=672).

### Example 49

### Synthesis of Compound 4-8

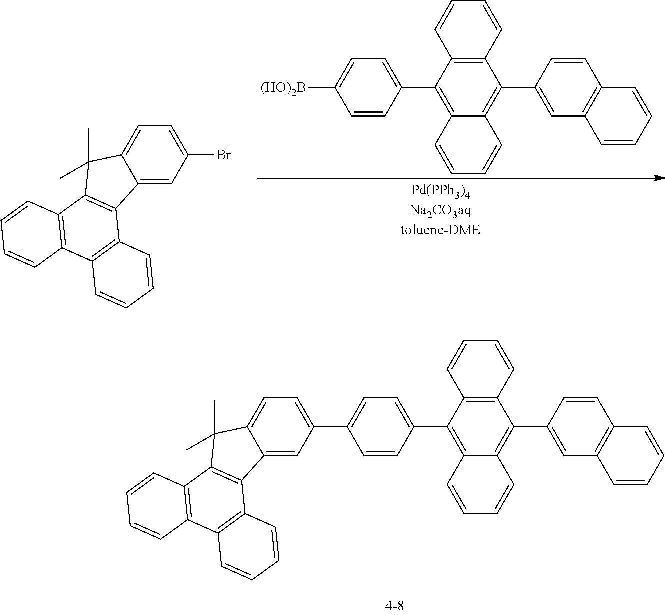

A compound 4-8 was synthesized in the same manner as in Example 1 according to the above scheme using the intermediate I. It was found by mass spectroscopy that the compound 4-8 was obtained. The compound 4-8 had a molecular weight of 672.28 (m/e=672).

### Example 50

### Synthesis of Compound 4-9

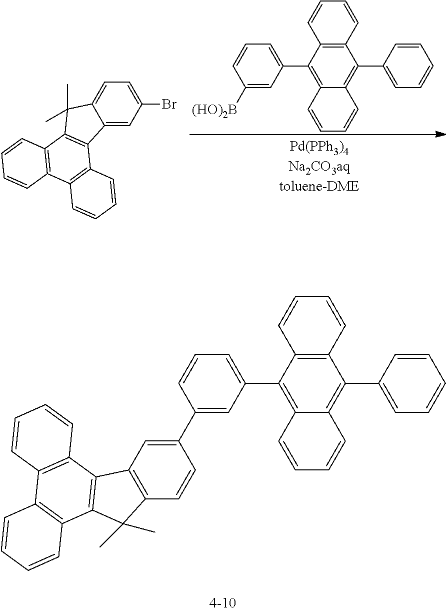

A compound 4-9 was synthesized in the same manner as in Example 1 according to the above scheme using the intermediate I. It was found by mass spectroscopy that the compound 4-9 was obtained. The compound 4-9 had a molecular weight of 622.27 (m/e=622).

### Example 51

### Synthesis of Compound 4-10

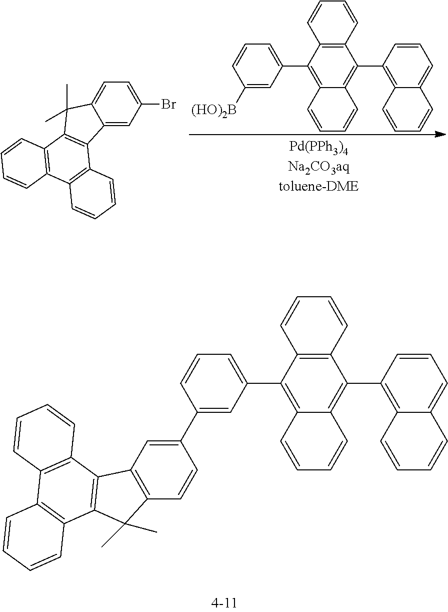

A compound 4-10 was synthesized in the same manner as in Example 1 according to the above scheme using the intermediate I. It was found by mass spectroscopy that the compound 4-10 was obtained. The compound 4-10 had a molecular weight of 672.28 (m/e=672).

### Example 52

### Synthesis of Compound 4-11

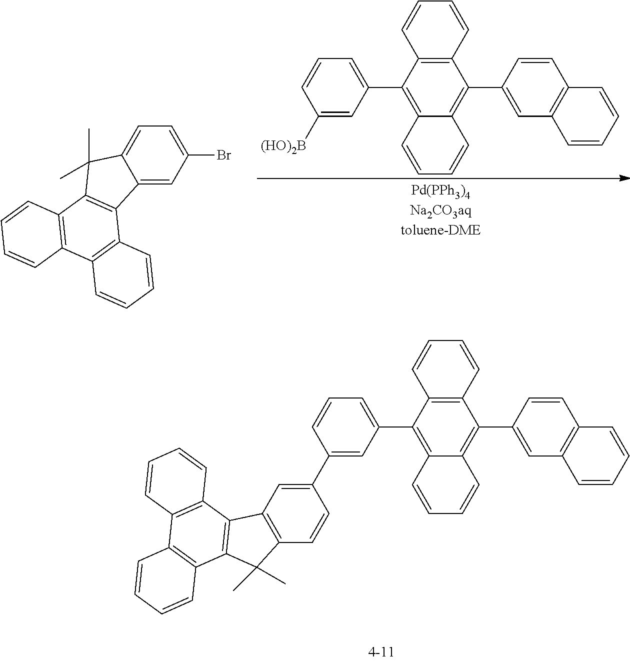

A compound 4-11 was synthesized in the same manner as in Example 1 according to the above scheme using the intermediate I. It was found by mass spectroscopy that the compound 4-11 was obtained. The compound 4-11 had a molecular weight of 672.28 (m/e=672).

### Example 53

### Synthesis of Compound 4-12

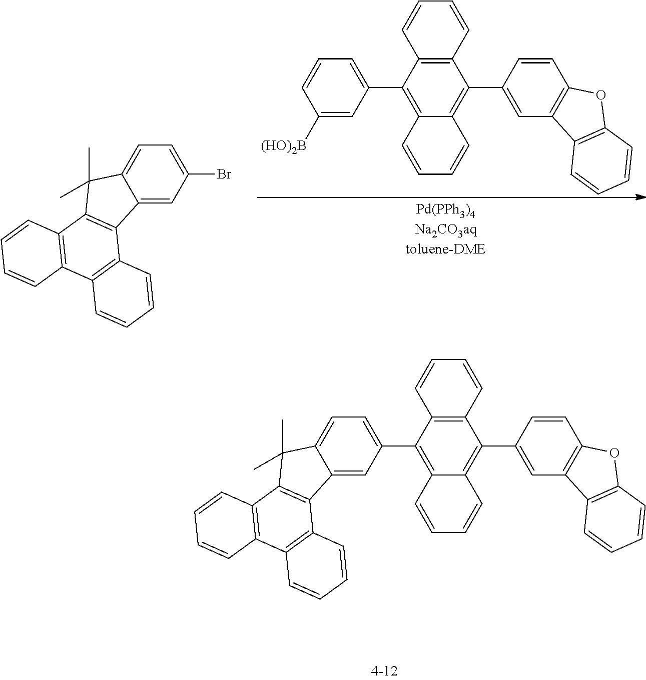 [SMILES: CC1(C)c2ccc(Br)cc2-c2c1c1ccccc1c1ccccc21 ; OB(O)c1cccc(-c2c3ccccc3c(-c3ccc4oc5ccccc5c4c3)c3ccccc23)c1 ; CC1(C)c2ccc(-c3c4ccccc4c(-c4ccc5oc6ccccc6c5c4)c4ccccc34)cc2-c2c1c1ccccc1c1ccccc21]

A compound 4-12 was synthesized in the same manner as in Example 1 according to the above scheme using the intermediate I. It was found by mass spectroscopy that the compound 4-12 was obtained. The compound 4-12 had a molecular weight of 636.25 (m/e=636).

### Example 54

### Synthesis of Compound 4-13

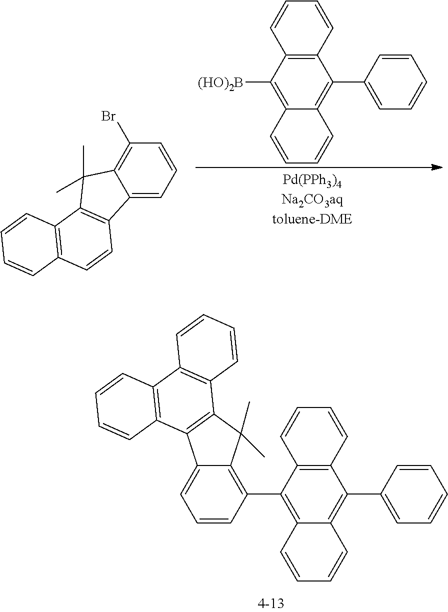

A compound 4-13 was synthesized in the same manner as in Example 1 according to the above scheme using the intermediate J. It was found by mass spectroscopy that the compound 4-13 was obtained. The compound 4-13 had a molecular weight of 546.23 (m/e=546).

### Example 55

A glass substrate provided with an ITO transparent electrode (anode) (25 mm×75 mm×1.1 mm (thickness)) (manufactured by Geomatics) was subjected to ultrasonic cleaning for 5 minutes in isopropyl alcohol, and subjected to UV ozone cleaning for 30 minutes. The glass substrate was then mounted on the substrate holder of a vacuum deposition device, and a compound HI-1 was deposited on the side of the glass substrate on which the linear transparent electrode was formed so as to cover the transparent electrode to form an HI-1 film having a thickness of 5 nm. A compound HT-1 was deposited on the HI-1 film to form an HT-1 film having a thickness of 80 nm. A compound HT-2 was deposited on the HT-1 film to form an HT-2 film having a thickness of 15 nm.

The compound 1-1 (emitting-layer host compound) and a dopant BD-1 were deposited on the HT-2 film in a thickness ratio of 19:1 to form an emitting layer having a thickness of 25 nm.

A compound ET-1 was deposited on the emitting layer to form an ET-1 film (electron-transporting layer) having a thickness of 20 nm. A compound ET-2 was deposited on the ET-1 film to form an ET-2 film having a thickness of 5 nm. LiF was deposited on the ET-2 film to form an LiF film having a thickness of 1 nm. Al metal was deposited on the LiF film to form a metal cathode having a thickness of 80 nm. An organic EL device was thus fabricated.

The resulting organic EL device was measured as to the voltage and the external quantum efficiency (EQE) as described below. The results are shown in Table 1.

### Driving Voltage

A voltage (V) was applied between the ITO transparent electrode and the Al metal cathode, and a voltage at which the current density was 10 mA/cm² was measured.

### External Quantum Efficiency (EQE)

The external quantum efficiency EQE (%) was calculated from the spectral radiance spectrum on the assumption that Lambertian radiation occurred.

### Examples 56 to 58 and Comparative Examples 1 to 4

An organic EL device was fabricated and evaluated in the same manner as in Example 55, except that the emitting layer was formed using the compound listed in Table 1 instead of the compound 1-1. The results are shown in Table 1.

The compounds used in Examples 55 to 58 and Comparative Examples 1 to 4 are shown below.

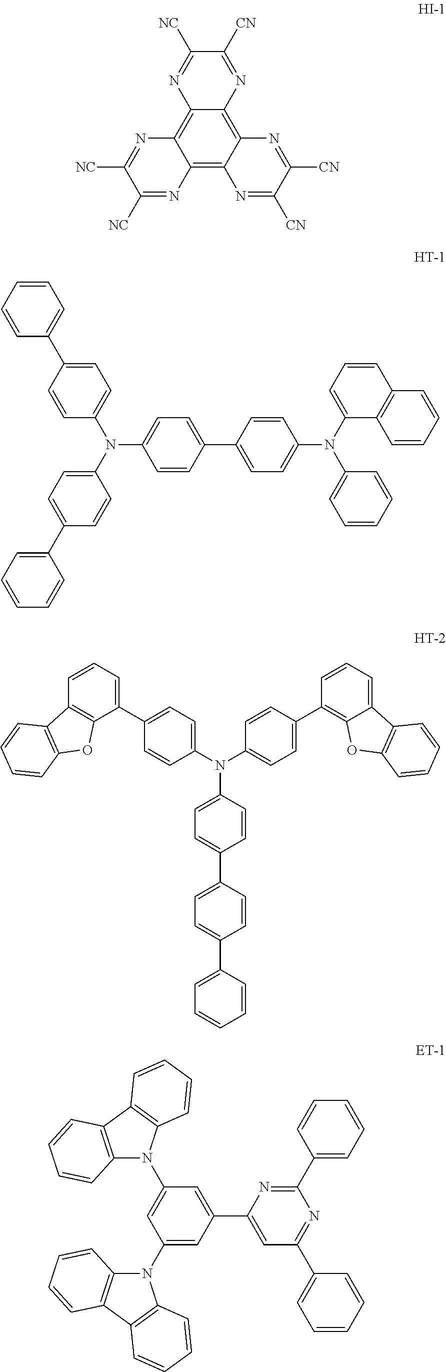 [SMILES: N#Cc1nc2c3nc(C#N)c(C#N)nc3c3nc(C#N)c(C#N)nc3c2nc1C#N ; c1ccc(-c2ccc(N(c3ccc(-c4ccccc4)cc3)c3ccc(-c4ccc(N(c5ccccc5)c5cccc6ccccc56)cc4)cc3)cc2)cc1 ; c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2cc(-c3cc(-n4c5ccccc5c5ccccc54)cc(-n4c5ccccc5c5ccccc54)c3)nc(-c3ccccc3)n2)cc1]

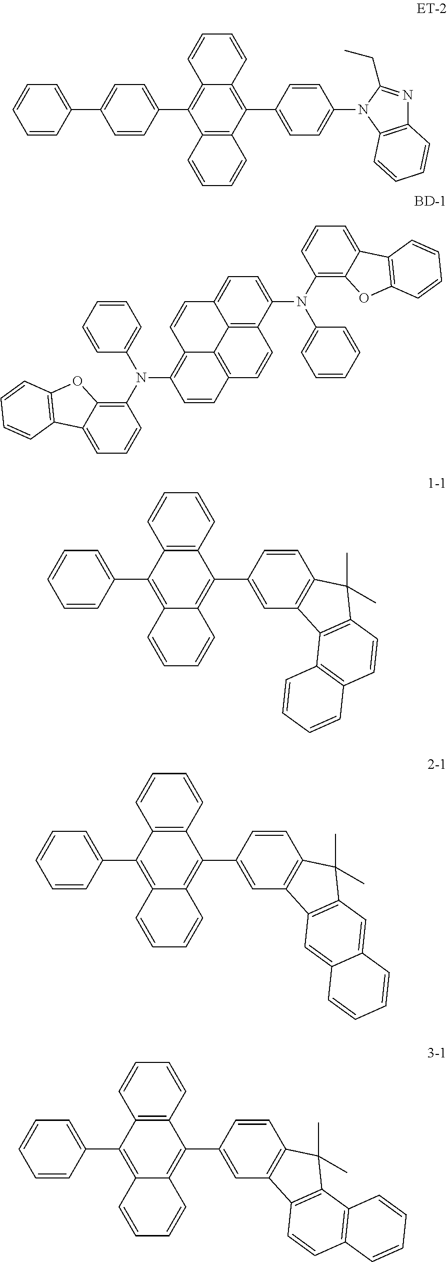

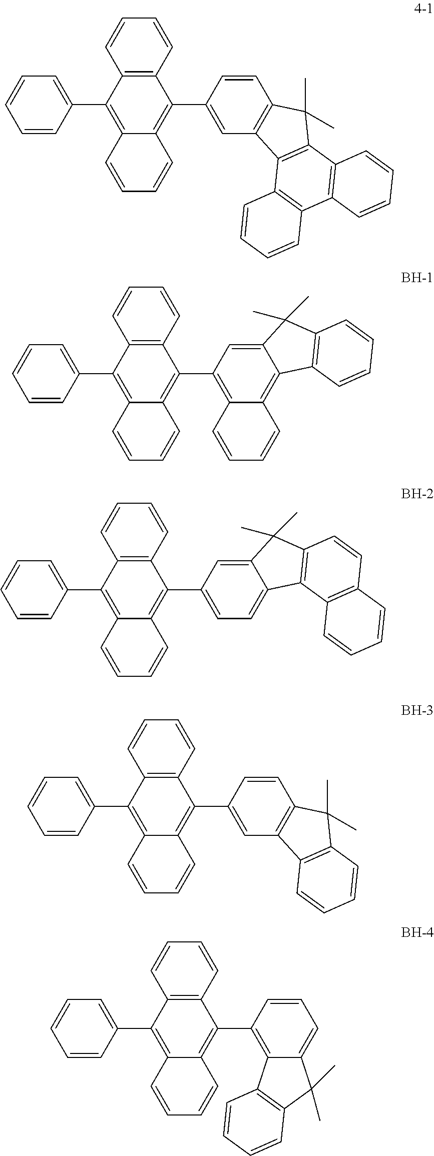

TABLE 1

| | Emitting-layer host | Voltage | EQE |
|---|---|---|---|
| | compound | (V) | (%) |
| Example 55 | 1-1 | 3.3 | 9.0 |
| Example 56 | 2-1 | 3.2 | 8.8 |
| Example 57 | 3-1 | 3.3 | 8.9 |
| Example 58 | 4-1 | 3.3 | 8.8 |
| Comparative Example 1 | BH-1 | 3.4 | 7.5 |
| Comparative Example 2 | BH-2 | 3.4 | 7.4 |
| Comparative Example 3 | BH-3 | 3.5 | 6.5 |
| Comparative Example 4 | BH-4 | 3.7 | 6.8 |

As is clear from the results shown in Table 1, the organic electroluminescence device fabricated using the compound according to the invention could be driven at a low voltage and exhibited high luminous efficiency. Such a decrease in voltage and an improvement in efficiency cannot be achieved using a known technique that changes the substitution position of fluorene and a known ring-fusing technique. It was confirmed that a material that makes it possible to specifically achieve a decrease in voltage while maintaining high efficiency can be obtained by bonding fused fluorene to an anthracene-containing structure at a specific position.

### Example 59

A glass substrate provided with an ITO transparent electrode (anode) (25 mm×75 mm×1.1 mm (thickness)) (manufactured by Geomatics) was subjected to ultrasonic cleaning for 5 minutes in isopropyl alcohol, and subjected to UV ozone cleaning for 30 minutes. The glass substrate was then mounted on the substrate holder of a vacuum deposition device, and a compound HI-1 was deposited on the side of the glass substrate on which the linear transparent electrode was formed so as to cover the transparent electrode to form an HI-1 film having a thickness of 5 nm. A compound HT-3 was deposited on the HI-1 film to form an HT-3 film having a thickness of 80 nm. A compound HT-4 was deposited on the HT-3 film to form an HT-4 film having a thickness of 15 nm.

The compound 1-1 (emitting-layer host compound) and a dopant BD-1 were deposited on the HT-4 film in a thickness ratio of 19:1 to form an emitting layer having a thickness of 25 nm.

A compound ET-3 and a compound ET-4 were deposited on the emitting layer in a thickness ratio of 1:1 to form an electron-transporting layer having a thickness of 25 nm. Al metal was deposited on the electron-transporting layer to form a metal cathode having a thickness of 80 nm. An organic EL device was thus fabricated.

The resulting organic EL device was measured as to the voltage and the external quantum efficiency (EQE) in the same manner as in Example 55. The results are shown in Table 2.

### Examples 60 to 85 and Comparative Examples 5 and 6

An organic EL device was fabricated and evaluated in the same manner as in Example 59, except that the emitting layer was formed using the compound listed in Table 2 instead of the compound 1-1. The results are shown in Table 2.

The compounds used in Examples 59 to 85 and Comparative Examples 5 and 6 are shown below.

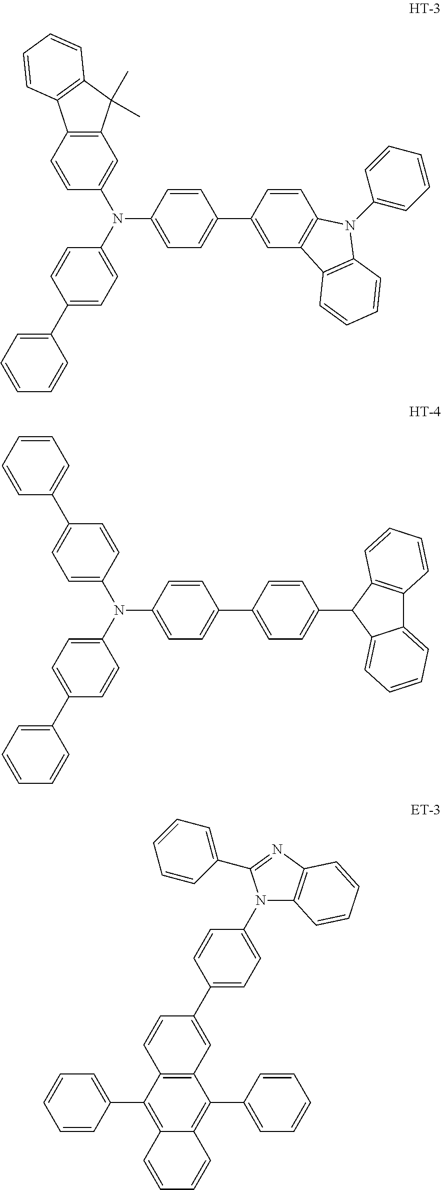

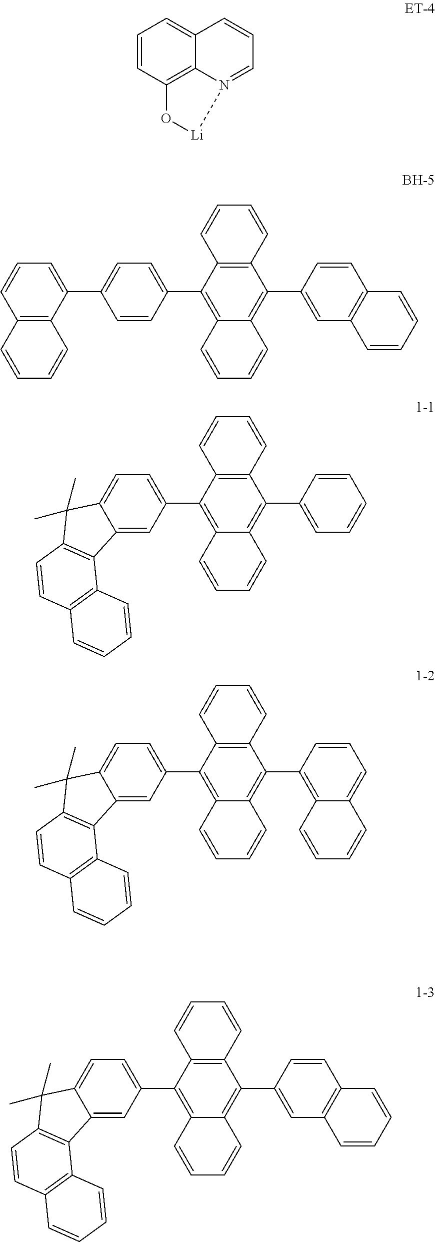

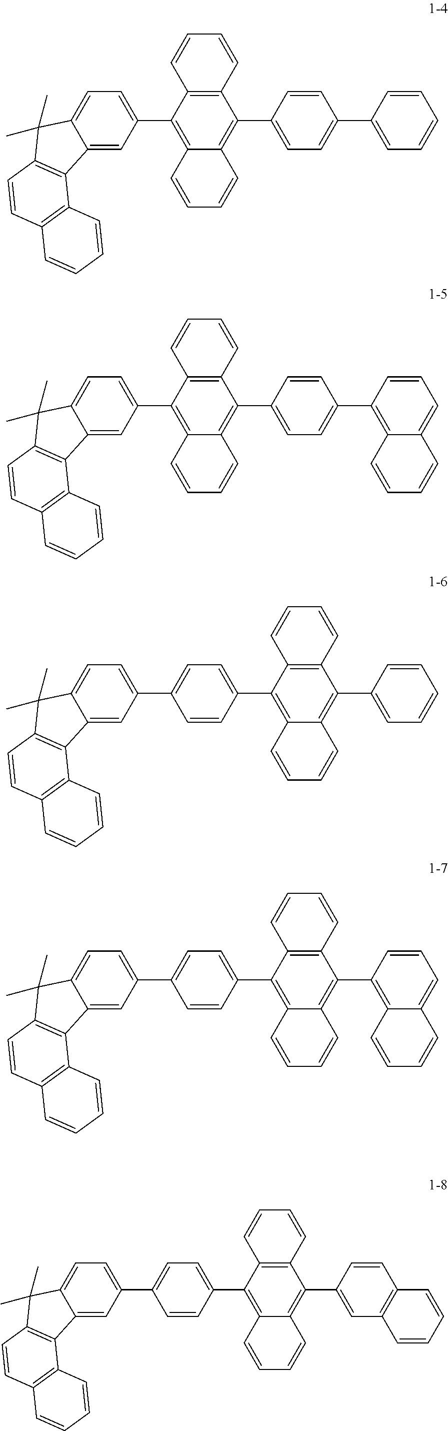

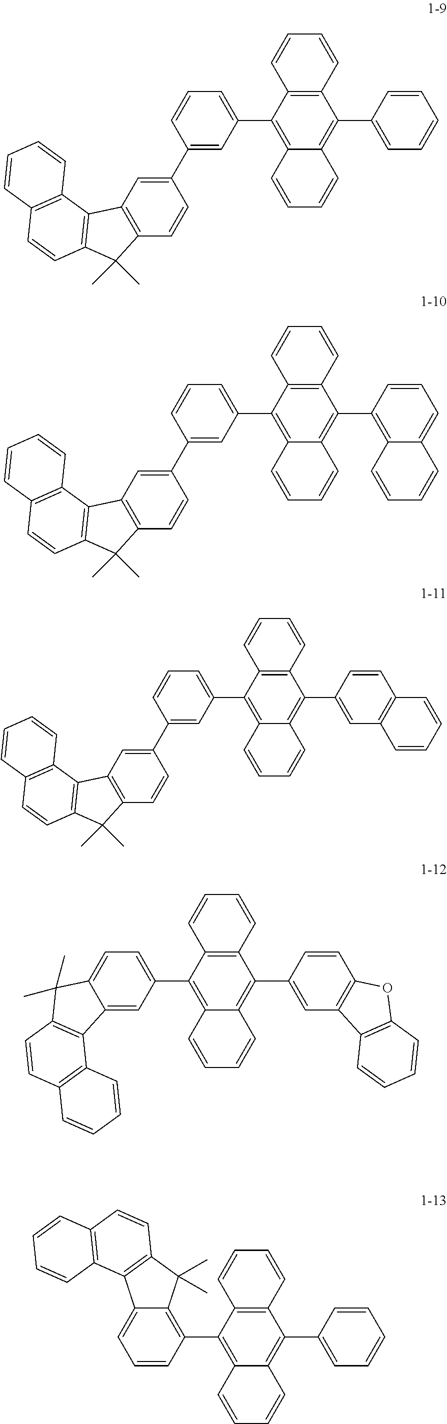

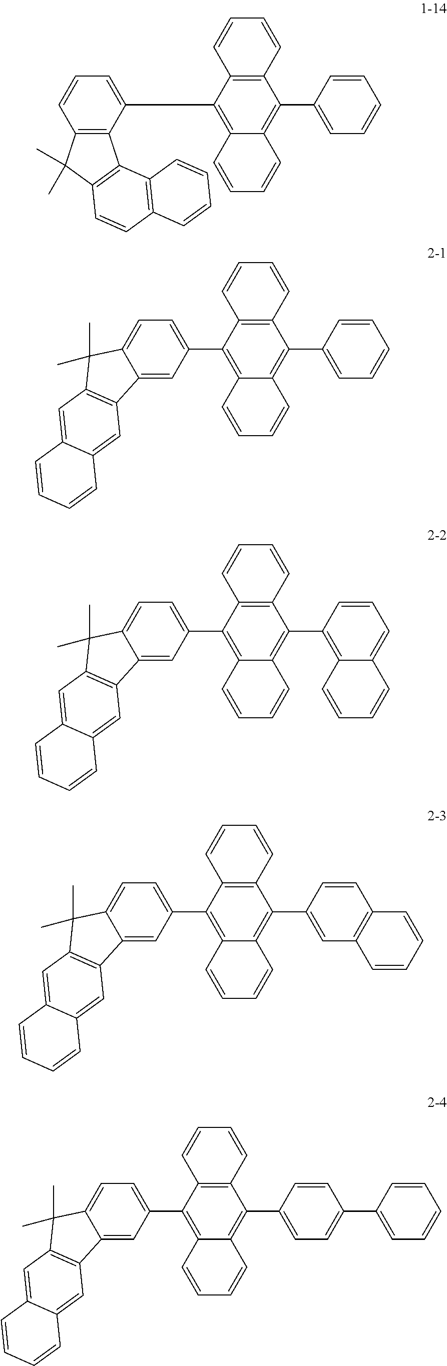

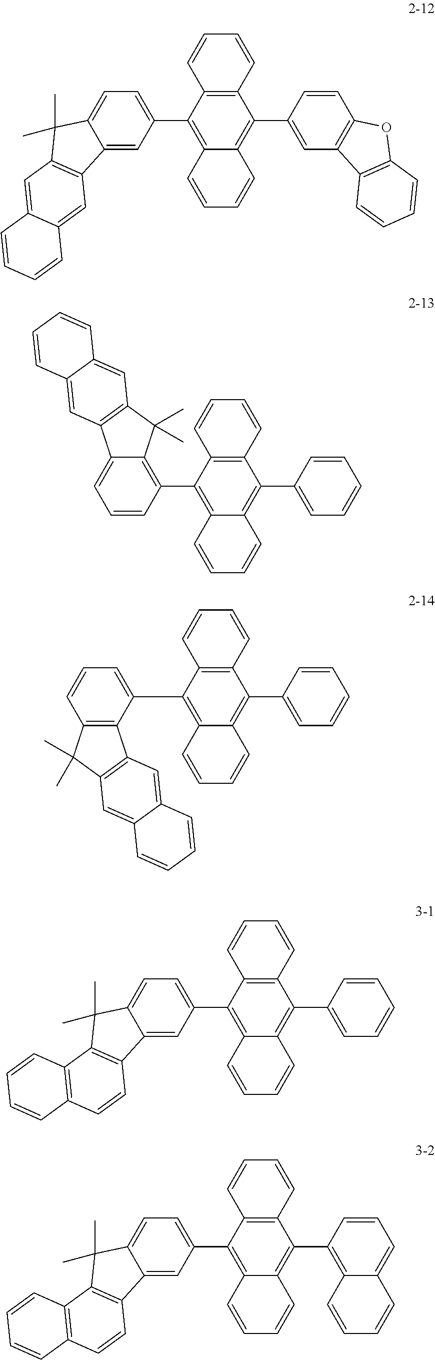

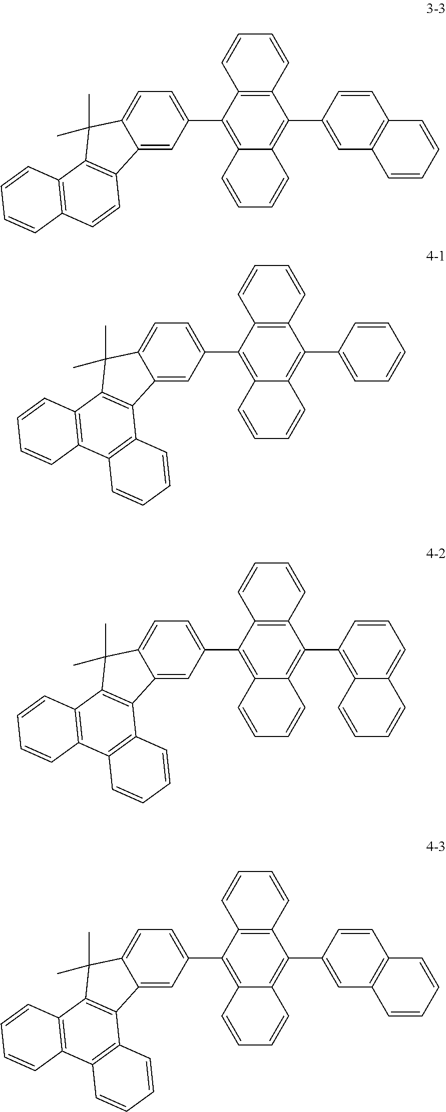

TABLE 2

| | Emitting layer host | Voltage | EQE |
|---|---|---|---|
| | compound | (V) | (%) |
| Example 59 | 1-1 | 3.5 | 8.3 |
| Example 60 | 1-2 | 3.4 | 8.2 |
| Example 61 | 1-3 | 3.4 | 8.4 |
| Example 62 | 1-4 | 3.4 | 8.3 |
| Example 63 | 1-5 | 3.3 | 8.4 |
| Example 64 | 1-6 | 3.4 | 8.3 |
| Example 65 | 1-7 | 3.3 | 8.5 |
| Example 66 | 1-8 | 3.3 | 8.4 |
| Example 67 | 1-9 | 3.6 | 8.0 |
| Example 68 | 1-10 | 3.5 | 8.1 |
| Example 69 | 1-11 | 3.5 | 8.1 |
| Example 70 | 1-12 | 3.3 | 7.9 |
| Example 71 | 1-13 | 3.6 | 8.1 |
| Example 72 | 1-14 | 3.6 | 8.2 |
| Example 73 | 2-1 | 3.4 | 8.1 |
| Example 74 | 2-2 | 3.3 | 8.3 |
| Example 75 | 2-3 | 3.3 | 8.1 |
| Example 76 | 2-4 | 3.3 | 8.4 |
| Example 77 | 2-12 | 3.3 | 8.0 |
| Example 78 | 2-13 | 3.6 | 7.9 |
| Example 79 | 2-14 | 3.6 | 8.0 |
| Example 80 | 3-1 | 3.5 | 8.2 |
| Example 81 | 3-2 | 3.4 | 8.4 |
| Example 82 | 3-3 | 3.4 | 8.3 |
| Example 83 | 4-1 | 3.5 | 8.1 |
| Example 84 | 4-2 | 3.4 | 8.2 |
| Example 85 | 4-3 | 3.4 | 8.1 |
| Comparative Example 5 | BH-1 | 3.6 | 6.8 |
| Comparative Example 6 | BH-5 | 3.7 | 6.6 |

Although only some exemplary embodiments and/or examples of the invention have been described in detail above, those skilled in the art will readily appreciate that many modifications are possible in the exemplary embodiments and/or examples without materially departing from the novel teachings and advantages of the invention. Accordingly, all such modifications are intended to be included within the scope of the invention.

The specification of the Japanese patent application to which the present application claims priority under the Paris Convention is incorporated herein by reference in its entirety.

1. An anthracene derivative represented by a formula (1), 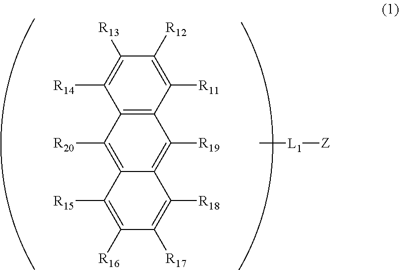
wherein in the formula (1), one of R₁₁ to R₂₀ is used to bond to L₁, and is a single bond,
the remainder of R₁₁ to R₂₀ that are not used to bond to L₁ are independently a hydrogen atom, a halogen atom, a cyano group, a substituted or unsubstituted alkyl group including 1 to 20 carbon atoms, a substituted or unsubstituted alkenyl group including 2 to 20 carbon atoms, a substituted or unsubstituted alkynyl group including 2 to 20 carbon atoms, a substituted or unsubstituted alkoxy group including 1 to 20 carbon atoms, a substituted or unsubstituted alkylthio group including 1 to 20 carbon atoms, a substituted or unsubstituted aryloxy group including 6 to 50 ring carbon atoms, a substituted or unsubstituted arylthio group including 6 to 50 ring carbon atoms, an alkylsilyl group that is substituted with a substituted or unsubstituted alkyl group including 1 to 20 carbon atoms, an arylsilyl group that is substituted with a substituted or unsubstituted aryl group including 6 to 50 ring carbon atoms, a substituted or unsubstituted aryl group including 6 to 50 ring carbon atoms, a substituted or unsubstituted heterocyclic group including 5 to 50 ring atoms, or a substituted or unsubstituted amino group,
provided that adjacent groups among R₁₁ to R₂₀ are optionally bonded to each other to form a ring,
L₁ is a single bond, a substituted or unsubstituted divalent aromatic hydrocarbon group including 6 to 50 ring carbon atoms, or a substituted or unsubstituted divalent heterocyclic group including 5 to 50 ring atoms, and
Z has a structure represented by a formula (2), 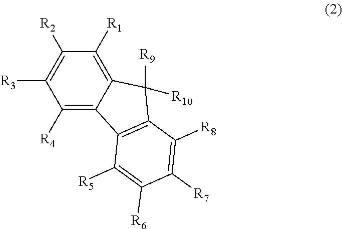
wherein in the formula (2), one of R₁, R₃, and R₄ is used to bond to L₁, and is a single bond, and
the remainder of R₁, R₃, and R₄ that are not bonded to L₁, R₂, and R₅ to R₁₀ are independently a hydrogen atom, a halogen atom, a cyano group, a substituted or unsubstituted alkyl group including 1 to 20 carbon atoms, a substituted or unsubstituted alkenyl group including 2 to 20 carbon atoms, a substituted or unsubstituted alkynyl group including 2 to 20 carbon atoms, a substituted or unsubstituted alkoxy group including 1 to 20 carbon atoms, a substituted or unsubstituted alkylthio group including 1 to 20 carbon atoms, a substituted or unsubstituted aryloxy group including 6 to 50 ring carbon atoms, a substituted or unsubstituted arylthio group including 6 to 50 ring carbon atoms, an alkylsilyl group that is substituted with a substituted or unsubstituted alkyl group including 1 to 20 carbon atoms, an arylsilyl group that is substituted with a substituted or unsubstituted aryl group including 6 to 50 ring carbon atoms, a substituted or unsubstituted aryl group including 6 to 50 ring carbon atoms, a substituted or unsubstituted heterocyclic group including 5 to 50 ring atoms, or a substituted or unsubstituted amino group,
provided that at least one pair of groups among R₅ to R₈ that are adjacent to each other are bonded to each other to form a saturated or unsaturated hydrocarbon ring, and
one of R₁, R₃, and R₄ is bonded directly to one of R₁₁ to R₂₀ when L₁ is a single bond.

2. The anthracene derivative according to claim 1, wherein at least one pair of groups among R₅ to R₈ that are adjacent to each other are bonded to each other to form a ring structure represented by a formula (3), 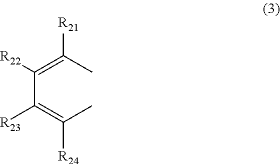
wherein in the formula (3), R₂₁ to R₂₄ are independently the same as defined above in connection with R₂ and R₅ to R₁₀ in the formula (2),
provided that adjacent groups among R₂₁ to R₂₄ are optionally bonded to each other to form a ring.

3. The anthracene derivative according to claim 1, wherein Z has a structure among structures respectively represented by formulas (4) to (7), 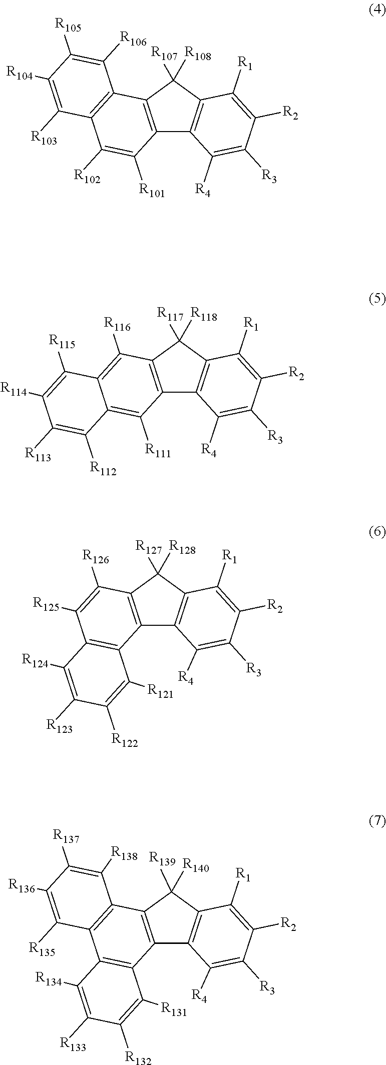
wherein in the formulas (4) to (7), one of R₁, R₃, and R₄ is used to bond to L₁, and is a single bond, and
the remainder of R₁, R₃, and R₄ that are not used to bond to L₁, R₂, R₁₀₁ to R₁₀₈, R₁₁₁ to R₁₁₈, R₁₂₁ to R₁₂₈, and R₁₃₁ to R₁₄₀ are independently the same as defined above in connection with R₂ and R₅ to R₁₀ in the formula (2).

4. The anthracene derivative according to claim 1, wherein at least one of R₁₁ to R₂₀ that is not used to bond to L₁ is a substituted or unsubstituted aryl group including 6 to 50 ring carbon atoms, or a substituted or unsubstituted heterocyclic group including 5 to 50 ring atoms.

5. The anthracene derivative according to claim 1, wherein R₂₀ is a substituted or unsubstituted aryl group including 6 to 50 ring carbon atoms, or a substituted or unsubstituted heterocyclic group including 5 to 50 ring atoms.

6. The anthracene derivative according to claim 1, the anthracene derivative being represented by any of formulas (8) to (11), 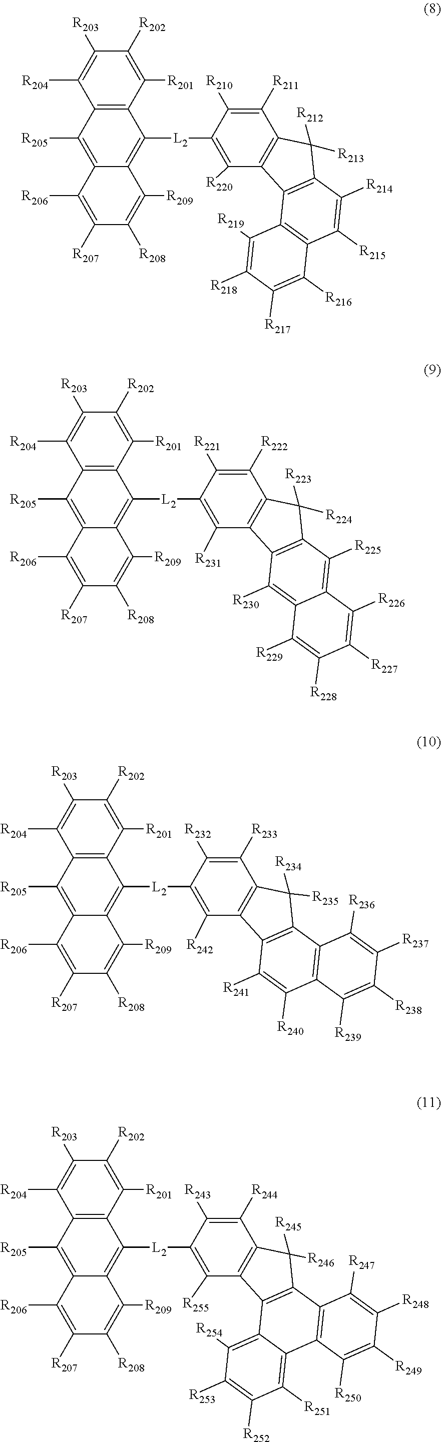
wherein in the formulas (8) to (11), R₂₀₁ to R₂₀₉ are independently the same as defined above in connection with R₁₁ to R₂₀ in the formula (1) that are not used to bond to L₁,
R₂₁₀ to R₂₂₀, R₂₂₁ to R₂₃₁, R₂₃₂ to R₂₄₂, and R₂₄₃ to R₂₅₅ are independently the same as defined above in connection with R₂ and R₅ to R₁₀ in the formula (1), and
L₂ is the same as defined above in connection with L₁ in the formula (1).

7. The anthracene derivative according to claim 6, wherein R₂₀₅ is a substituted or unsubstituted aryl group including 6 to 50 ring carbon atoms, or a substituted or unsubstituted heterocyclic group including 5 to 50 ring atoms.

8. The anthracene derivative according to claim 1, the anthracene derivative being represented by any of formulas (12) to (15), 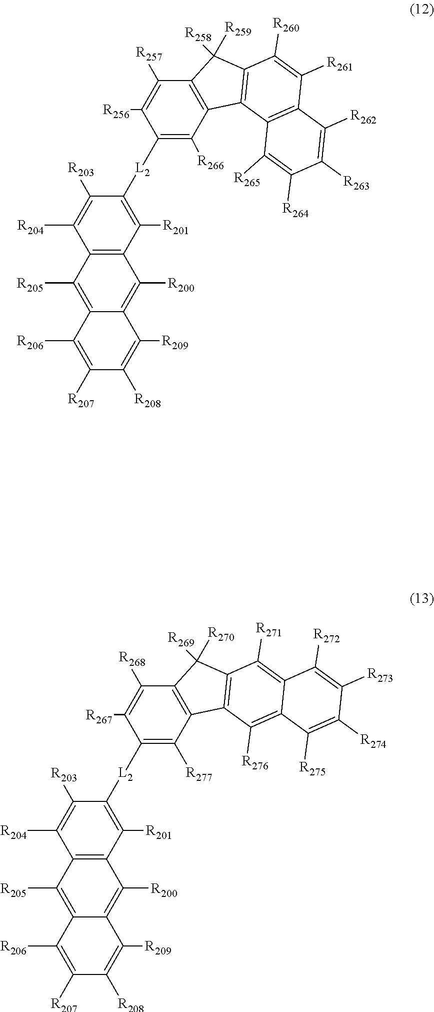 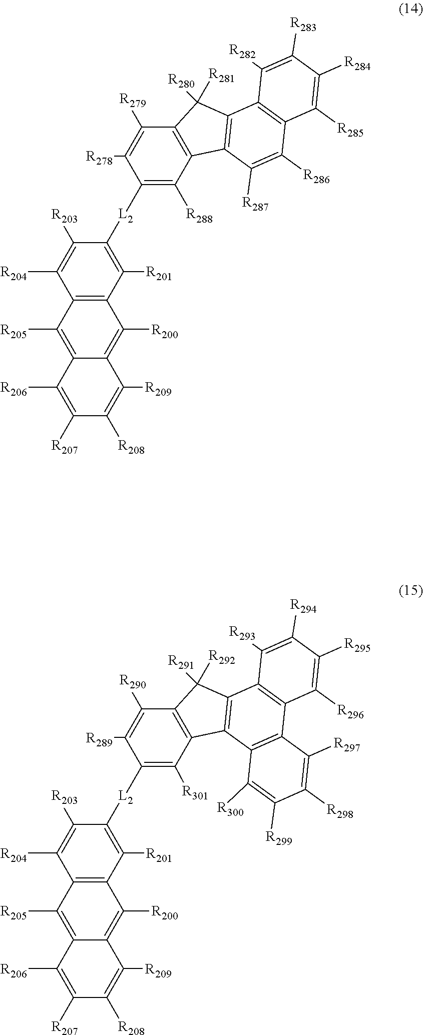
wherein in the formulas (12) to (15), R₂₀₀, R₂₀₁, and R₂₀₃ to R₂₀₉ are independently the same as defined above in connection with R₁₁ to R₂₀ in the formula (1) that are not used to bond to Lᵢ,
R₂₅₆ to R₂₆₆, R₂₆₇ to R₂₇₇, R₂₇₈ to R₂₈₈, and R₂₈₉ to R₃₀₁ are independently the same as defined above in connection with R₂ and R₅ to R₁₀ in the formula (2), and
L₂ is the same as defined above in connection with L₁ in the formula (1).

9. The anthracene derivative according to claim 8, wherein one or more selected from R₂₀₀ and R₂₀₅ are independently a substituted or unsubstituted aryl group including 6 to 50 ring carbon atoms, or a substituted or unsubstituted heterocyclic group including 5 to 50 ring atoms.

10. A material for producing an organic electroluminescence device comprising the anthracene derivative according to claim 1.

11. An organic electroluminescence device comprising a cathode, an anode, and one or more organic thin film layers that are provided between the cathode and the anode, the one or more organic thin film layers including an emitting layer, and at least one organic thin film layer included in the one or more organic thin film layers comprising the anthracene derivative according to claim 1 either alone or as a component of a mixture.

12. The organic electroluminescence device according to claim 11, wherein the emitting layer comprises the anthracene derivative.

13. The organic electroluminescence device according to claim 12, wherein the anthracene derivative is a host material.

14. The organic electroluminescence device according to claim 12, wherein the emitting layer further comprises at least one of a fluorescent dopant and a phosphorescent dopant.

15. The organic electroluminescence device according to claim 14, wherein the fluorescent dopant is an arylamine compound.

16. The organic electroluminescence device according to claim 15, wherein the fluorescent dopant is a fused-ring amine derivative represented by a formula (16), 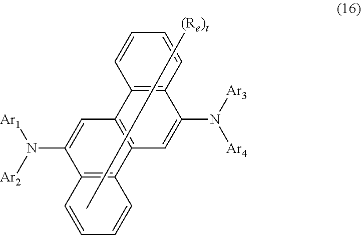
wherein in the formula (16), Rₑ is independently a substituted or unsubstituted alkyl group including 1 to 20 carbon atoms, a substituted or unsubstituted alkenyl group including 2 to 50 carbon atoms, a substituted or unsubstituted alkynyl group including 2 to 50 carbon atoms, a substituted or unsubstituted aralkyl group including 7 to 50 carbon atoms, a substituted or unsubstituted cycloalkyl group including 3 to 20 ring carbon atoms, a substituted or unsubstituted alkoxy group including 1 to 20 carbon atoms, a substituted or unsubstituted aryloxy group including 6 to 20 ring carbon atoms, a substituted or unsubstituted aryl group including 6 to 50 ring carbon atoms, a substituted or unsubstituted heterocyclic group including 5 to 50 ring atoms, a substituted or unsubstituted alkylsilyl group including 1 to 30 carbon atoms, a substituted or unsubstituted arylsilyl group including 6 to 50 ring carbon atoms, a substituted or unsubstituted alkylgermanium group including 1 to 50 carbon atoms, or a substituted or unsubstituted arylgermanium group including 6 to 50 ring carbon atoms, provided that Rₑ is bonded to an arbitrary position of the 4-ring fused skeleton in the formula (16),
t is an integer from 1 to 10,
provided that a plurality of Rₑ are either identical or different when t is an integer from 2 to 10, and
Ar₁ to Ar₄ are independently a substituted or unsubstituted aryl group including 6 to 50 ring carbon atoms, or a substituted or unsubstituted heterocyclic group including 5 to 50 ring atoms.

17. The organic electroluminescence device according to claim 15, wherein the fluorescent dopant is a fused-ring amine derivative represented by a formula (17), 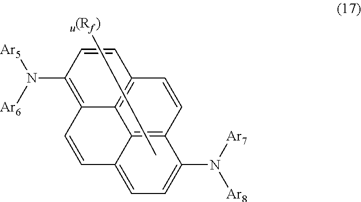
wherein in the formula (17), R_{f} is independently a substituted or unsubstituted alkyl group including 1 to 20 carbon atoms, a substituted or unsubstituted alkenyl group including 2 to 50 carbon atoms, a substituted or unsubstituted alkynyl group including 2 to 50 carbon atoms, a substituted or unsubstituted aralkyl group including 7 to 50 carbon atoms, a substituted or unsubstituted cycloalkyl group including 3 to 20 ring carbon atoms, a substituted or unsubstituted alkoxy group including 1 to 20 carbon atoms, a substituted or unsubstituted aryloxy group including 6 to 20 ring carbon atoms, a substituted or unsubstituted aryl group including 6 to 50 ring carbon atoms, a substituted or unsubstituted heterocyclic group including 5 to 50 ring atoms, a substituted or unsubstituted alkylsilyl group including 1 to 30 carbon atoms, a substituted or unsubstituted arylsilyl group including 6 to 50 ring carbon atoms, a substituted or unsubstituted alkylgermanium group including 1 to 50 carbon atoms, or a substituted or unsubstituted arylgermanium group including 6 to 50 ring carbon atoms, provided that R_{f} is bonded to an arbitrary position of the 4-ring fused skeleton in the formula (17),
u is an integer from 0 to 8,
provided that a plurality of R_{f} are either identical or different when u is an integer from 2 to 8, and
Ar₅ to Ar₈ are independently a substituted or unsubstituted aryl group including 6 to 50 ring carbon atoms, or a substituted or unsubstituted heterocyclic group including 5 to 50 ring atoms.

18. The organic electroluminescence device according to claim 15, wherein the fluorescent dopant is a fused-ring amine derivative represented by a formula (18), 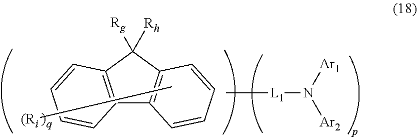
wherein in the formula (18), R_{g} and Rₕ are independently a hydrogen atom, a substituted or unsubstituted alkyl group including 1 to 20 carbon atoms, a substituted or unsubstituted alkenyl group including 2 to 50 carbon atoms, a substituted or unsubstituted alkynyl group including 2 to 50 carbon atoms, a substituted or unsubstituted aralkyl group including 7 to 50 carbon atoms, a substituted or unsubstituted cycloalkyl group including 3 to 20 ring carbon atoms, a substituted or unsubstituted alkoxy group including 1 to 20 carbon atoms, a substituted or unsubstituted aryloxy group including 6 to 20 ring carbon atoms, a substituted or unsubstituted aryl group including 6 to 50 ring carbon atoms, a substituted or unsubstituted heterocyclic group including 5 to 50 ring atoms, a substituted or unsubstituted alkylsilyl group including 1 to 30 carbon atoms, a substituted or unsubstituted arylsilyl group including 6 to 50 ring carbon atoms, a substituted or unsubstituted alkylgermanium group including 1 to 50 carbon atoms, or a substituted or unsubstituted arylgermanium group including 6 to 50 ring carbon atoms,
Rᵢ is a substituted or unsubstituted alkyl group including 1 to 20 carbon atoms, a substituted or unsubstituted alkenyl group including 2 to 50 carbon atoms, a substituted or unsubstituted alkynyl group including 2 to 50 carbon atoms, a substituted or unsubstituted aralkyl group including 7 to 50 carbon atoms, a substituted or unsubstituted cycloalkyl group including 3 to 20 ring carbon atoms, a substituted or unsubstituted alkoxy group including 1 to 20 carbon atoms, a substituted or unsubstituted aryloxy group including 6 to 20 ring carbon atoms, a substituted or unsubstituted aryl group including 6 to 50 ring carbon atoms, a substituted or unsubstituted heterocyclic group including 5 to 50 ring atoms, a substituted or unsubstituted alkylsilyl group including 1 to 30 carbon atoms, a substituted or unsubstituted arylsilyl group including 6 to 50 ring carbon atoms, a substituted or unsubstituted alkylgermanium group including 1 to 50 carbon atoms, or a substituted or unsubstituted arylgermanium group including 6 to 50 ring carbon atoms, provided that Rᵢ is bonded to an arbitrary position of the fluorene skeleton in the formula (18),
q is an integer from 0 to 7, provided that a plurality of Rᵢ are either identical or different, and adjacent Rᵢ are optionally bonded to each other to form a ring when q is an integer from 2 to 7,
L₁ is a single bond or a linking group, provided that L₁ is bonded to the fluorene skeleton in the formula (18) at a position at which Rᵢ is not bonded,
Ar₁ and Ar₂ are independently a substituted or unsubstituted aryl group including 6 to 50 ring carbon atoms, or a substituted or unsubstituted heterocyclic group including 5 to 50 ring atoms, and
p is an integer from 1 to 4.

19. An electronic device comprising the organic electroluminescence device according to claim 11.
